(19) United States
(12) Patent Application Publication
(10) Pub. No.: US 2023/0374015 A1
(43) Pub. Date: Nov. 23, 2023

(54) INHIBITORS OF FIBROBLAST GROWTH FACTOR RECEPTOR KINASES

(71) Applicant: Kinnate Biopharma Inc., San Diego, CA (US)

(72) Inventors: Stephen W. KALDOR, San Diego, CA (US); John TYHONAS, San Diego, CA (US); Eric A. MURPHY, San Marcos, CA (US); Toufike KANOUNI, Rancho Santa Fe, CA (US); Lee D. ARNOLD, Rancho Santa Fe, CA (US); Robert KANIA, Del Mar, CA (US)

(21) Appl. No.: 18/000,616

(22) PCT Filed: Jun. 4, 2021
(86) PCT No.: PCT/US2021/035856

§ 371 Date: Jul. 28, 2023

Related U.S. Application Data
(60) Provisional application No. 63/035,243, filed on Jun. 5, 2020.

(51) Int. Cl. C07D 487/04; C07D 519/00; C07D 471/04

(52) U.S. Cl. CPC C07D 487/04; C07D 519/00; C07D 471/04; C07B 2200/05

(57) ABSTRACT

Provided herein are heteroaryl inhibitors of fibroblast growth factor receptor kinases, pharmaceutical compositions comprising said compounds, and methods for using said compounds for the treatment of diseases.

### CROSS-REFERENCE TO RELATED APPLICATION

This application claims benefit of U.S. Patent Application No. 63/035,243, filed on Jun. 5, 2020, which is hereby incorporated by reference in its entirety.

### BACKGROUND

Fibroblast growth factor receptors (FGFRs) are a subfamily of receptor tyrosine kinases (RTKs) that bind to members of the fibroblast growth factor family of proteins. Deregulation of the fibroblast growth factor/FGF receptor network occurs frequently in tumors. Accordingly, therapies that target abberant FGFR kinase activity are desired for use in the treatment of cancer and other disorders.

### BRIEF SUMMARY OF THE INVENTION

Provided herein are inhibitors of fibroblast growth factor receptor (FGFR) kinases, pharmaceutical compositions comprising said compounds, and methods for using said compounds for the treatment of diseases.

One embodiment provides a compound, or pharmaceutically acceptable salt or solvate thereof, having the structure of Formula (I):

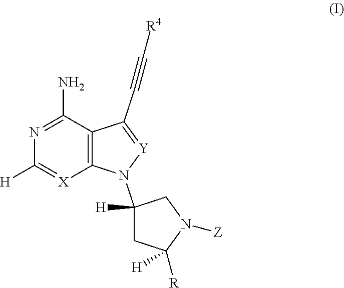

wherein,
X is C—H or N;
Y is C—H or N;
Z is selected from a group having the structure:

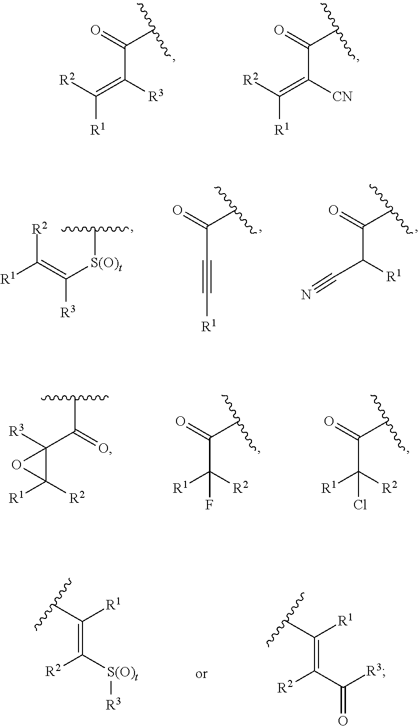

t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

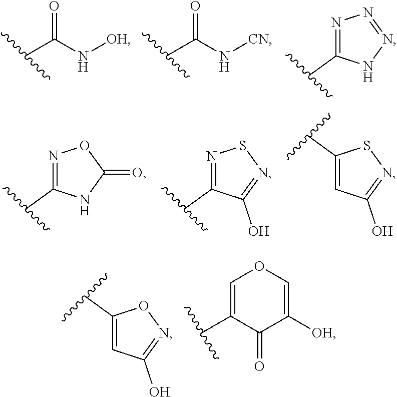

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

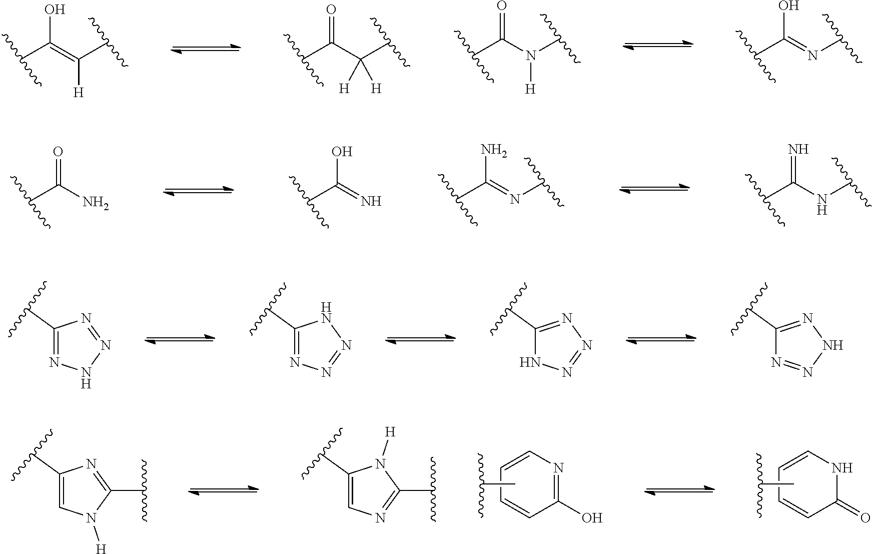

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

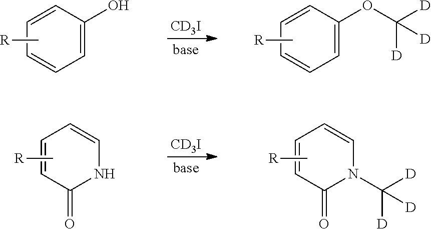

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

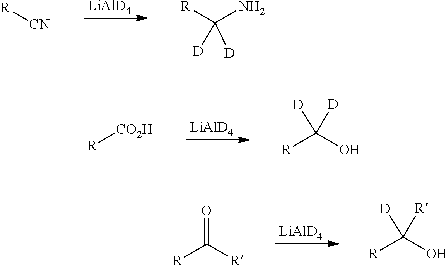

Deuterium gas and palladium catalyst are employed to reduce unsaturated carbon-carbon linkages and to perform a reductive substitution of aryl carbon-halogen bonds as illustrated, by way of example only, in the reaction schemes below.

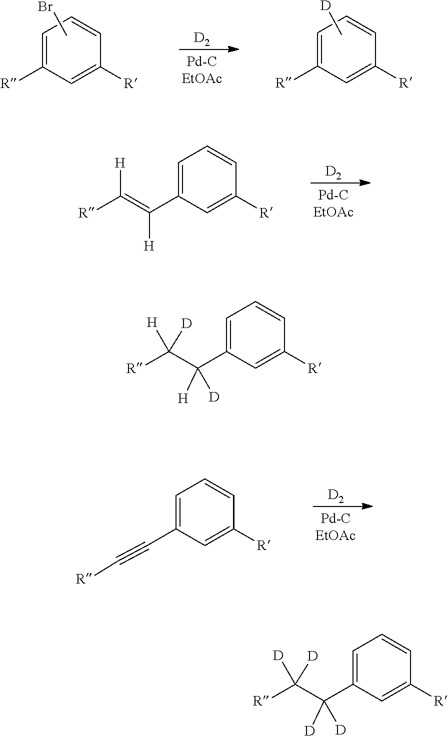

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of fibroblast growth factor receptors (FGFRs) compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Fibroblast Growth Factor Receptor (FGFR)

Fibroblast growth factor receptors (FGFRs) are a subfamily of receptor tyrosine kinases (RTKs) that bind to members of the fibroblast growth factor family of proteins. FGFR genes generally contain 18 exons, possess similar exon-intron organization, and are randomly dispersed throughout the genome with no apparent linkages to FGF gene locations. FGFRs are differentially expressed in a tissue-specific manner throughout development and into adulthood and comprise an extracellular ligand-binding domain, a single-transmembrane domain, and a split intracellular kinase domain. The extracellular region contains two to three immunoglobulin (Ig)-like domains that are involved in FGF binding. These Ig-like domains regulate both ligand affinity and ligand specificity. The intracellular region has the functional domain responsible for FGFR tyrosine kinase activity, as well as additional sites that play a role in protein binding and phosphorylation or autophosphorylation of the receptor molecule. Fibroblast grouth factor receptor pharmacology has been reviewed in the scientific literature by Porta et al. (Criticial Reviews in Oncology/Hematology 113 (2017) 256-67) and Babina and Turner (Nature Review-Cancer 2017 doi: 10.1038/nrc.2017.8).

The FGFR family comprises of four family members—FGFR1, FGFR2, FGFR3, and FGFR4, but the four members are capable of producing multiple receptor isoforms through alternative splicing of primary transcripts. A closely-related receptor which lacks the FGF signaling tyrosine kinase domain, FGFR5, (also known as FGFRL1) was recently discovered on the basis of interaction with FGFR-binding ligands, known as fibroblast growth factors (FGFs) (Trueb B. Biology of FGFRL1, the fifth fibroblast growth factor receptor. Cell Mol Life Sci. 2011; 68(6):951-964). Collectively, FGFR signaling is associated with the activation of multiple cellular cascades and responses such as cell growth, proliferation, differentiation, and survival (Thisse B et al. Functions and regulations of fibroblast growth factor signaling during embryonic development. Dev Biol. 2005; 287(2):390-402; Wesche J et al. Fibroblast growth factors and their receptors in cancer. Biochem J. 2011; 437(2):199-213; Haugsten E M et al. Roles of fibroblast growth factor receptors in carcinogenesis. Mol Cancer Res. 2010; 8(11):1439-1452).

Numerous human pathological conditions are associated with the deregulation of FGFR signaling. Aberrant FGFR signaling is largely attributed to several underlying mechanisms involving gene amplification, gain-of-function coding mutation, gene fusions, single nucleotide polymorphism (SNP), ligand availability and impaired termination program in FGF-mediated signaling (Tiong K H et al. Functional roles of fibroblast growth factor receptors (FGFRs) signaling in human cancers. Apoptosis. 2013; 18(12):1447-68). In addition, a further layer of complexity is added by the fact that FGFRs are subjected to alternative splicing, giving rise to multiple isoforms which may promote or repress tumorigenesis, under different circumstances.

FGFR Fusions

FGFR fusions in human cancers are classified into type 1 fusions caused by chromosomal translocations in hematological malignancies, and type 2 fusions caused by chromosomal rearrangements in solid tumors (FGFR inhibitors: Effects on cancer cells, tumor microenvironment and whole-body homeostasis (Review). Int J Mol Med. 2016; 38(1):3-15). Both types of FGFR fusion proteins are endowed with oncogenic potential through the acquisition of protein-protein-interaction modules from fusion partners for ligand-independent dimerization and/or recruitment of aberrant substrates. Human FGFR fusion proteins generally consist of two main segments—the anterior being a dimerized domain from a partnering gene and tyrosine kinase domain at the posterior (Garcia-Closas M et al. Heterogeneity of breast cancer associations with five susceptibility loci by clinical and pathological characteristics. PLoS Genet. 2008; 4 (4):e1000054). Unlike wild type receptors, mutant FGFRs are expressed intracellularly and retained in the cytosol, thus they escape the typical receptor degradation processes, further prolonging the activation signal.

Deregulation of the fibroblast growth factor (FGF)/FGF receptor (FGFR) network occurs frequently in tumors, resulting in the development of FGF/FGFR-targeting therapies as the focus of several basic, preclinical, and clinical studies.

Heteroaromatic FGFR Inhibitory Compounds

In one aspect, provided herein is a heteroaromatic FGFR inhibitory compound.

One embodiment provides a compound, or pharmaceutically acceptable salt or solvate thereof, having the structure of Formula (I):

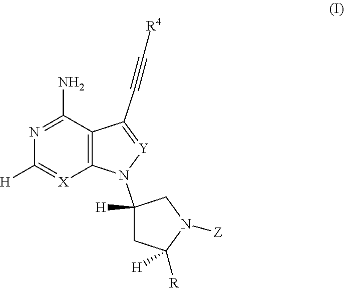

wherein,
X is C—H or N;
Y is C—H or N;
Z is selected from a group having the structure:

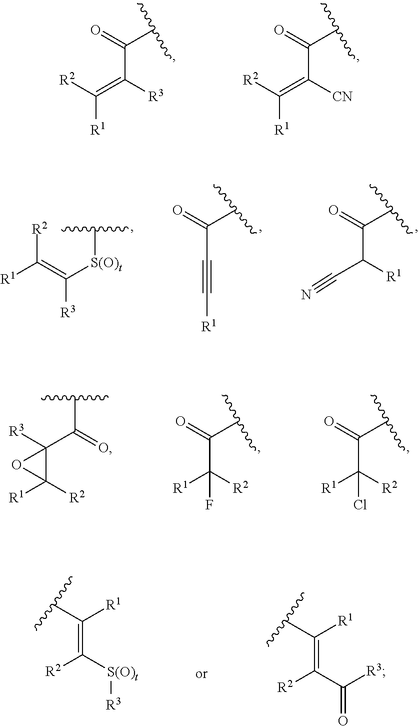

t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

One embodiment provides a compound of Formula (I), or a pharmaceutically acceptable salt or solvate thereof, wherein X is C—H, and Y is C—H.

One embodiment provides a compound of Formula (I), or a pharmaceutically acceptable salt or solvate thereof, wherein X is C—H, and Y is N.

One embodiment provides a compound of Formula (I), or a pharmaceutically acceptable salt or solvate thereof, wherein X is N, and Y is C—H.

One embodiment provides a compound of Formula (I), or a pharmaceutically acceptable salt or solvate thereof, wherein X is N, and Y is N.

One embodiment provides a compound of Formula (I), or a pharmaceutically acceptable salt or solvate thereof, wherein Z is

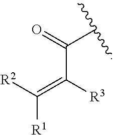

Another embodiment provides the compound, or pharmaceutically acceptable salt or solvate thereof, wherein R² is hydrogen. Another embodiment provides the compound, or pharmaceutically acceptable salt or solvate thereof, wherein R³ is hydrogen or fluoro. Another embodiment provides the compound, or pharmaceutically acceptable salt or solvate thereof, wherein R² and R³ are hydrogen. Another embodiment provides the compound, or pharmaceutically acceptable salt or solvate thereof, wherein R¹ is hydrogen. Another embodiment provides the compound, or pharmaceutically acceptable salt or solvate thereof, wherein R¹ is optionally substituted C1-C4 alkyl. Another embodiment provides the compound, or pharmaceutically acceptable salt or solvate thereof, wherein R¹ is optionally substituted C1-C2 alkyl. Another embodiment provides the compound, or pharmaceutically acceptable salt or solvate thereof, wherein R¹ is optionally substituted C1 alkyl. Another embodiment provides the compound, or pharmaceutically acceptable salt or solvate thereof, wherein the optionally substituted alkyl is substituted with an optionally substituted amino group. Another embodiment provides the compound, or pharmaceutically acceptable salt or solvate thereof, wherein the optionally substituted amino group is a dimethylamino.

One embodiment provides a compound of Formula (I), or a pharmaceutically acceptable salt or solvate thereof, wherein R⁴ is selected from an optionally substituted nitrogen-containing 9 or 10-atom heteroaryl is selected from optionally substituted benzimidazole, optionally substituted 1H-indazole, optionally substituted 2H-indazole, optionally substituted benzotriazole, optionally substituted benzoxazole, optionally substituted imidazo[4,5-c]pyridine, or optionally substituted imidazo[4,5-b]pyridine. One embodiment provides a compound of Formula (I), or a pharmaceutically acceptable salt or solvate thereof, wherein R⁴ is selected from an optionally substituted nitrogen-containing 9 or 10-atom heteroaryl is selected from quinoline, quinoxaline, pyrazolo[1,5-a]pyrimidine, imidazo[1,2-a]pyridine, pyrazolo[1,5-a]pyridine, imidazo[1,2-a]pyrimidine, imidazo[1,2-b]pyridazine, or pyrazolo[1,5-a]pyridine. Another embodiment provides the compound, or a pharmaceutically acceptable salt or solvate thereof, wherein R⁴ is an optionally substituted benzimidazole. Another embodiment provides the compound, or a pharmaceutically acceptable salt or solvate thereof, wherein R⁴ is an optionally substituted 1H-indazole. Another embodiment provides the compound, or a pharmaceutically acceptable salt or solvate thereof, wherein R⁴ is an optionally substituted 2H-indazole. Another embodiment provides the compound, or a pharmaceutically acceptable salt or solvate thereof, wherein R⁴ is an optionally substituted benzoxazole, optionally substituted imidazo[4,5-c]pyridine, or optionally substituted imidazo[4,5-b]pyridine. Another embodiment provides the compound, or a pharmaceutically acceptable salt or solvate thereof, wherein optionally substituted nitrogen-containing 9 or 10-atom heteroaryl is optionally substituted with alkyl, cycloalkyl, or halogen. Another embodiment provides the compound, or a pharmaceutically acceptable salt or solvate thereof, wherein the optionally substituted benzimidazole is optionally substituted with alkyl, cycloalkyl, or halogen. Another embodiment provides the compound, or a pharmaceutically acceptable salt or solvate thereof, wherein the optionally substituted 1H-indazole is optionally substituted with alkyl, cycloalkyl, or halogen. Another embodiment provides the compound, or a pharmaceutically acceptable salt or solvate thereof, wherein the optionally substituted 2H-indazole is optionally substituted with alkyl, cycloalkyl, or halogen.

One embodiment provides a compound of Formula (I), or a pharmaceutically acceptable salt or solvate thereof, wherein R is hydrogen. Another embodiment provides the compound, or a pharmaceutically acceptable salt or solvate thereof, wherein R is optionally substituted C1-C6 alkyl. Another embodiment provides the compound, or a pharmaceutically acceptable salt or solvate thereof, wherein R is optionally substituted C3-C7 carbocyclyl. Another embodiment provides the compound, or a pharmaceutically acceptable salt or solvate thereof, wherein R is optionally substituted C3-C7 carbocyclylalkyl. Another embodiment provides the compound, or a pharmaceutically acceptable salt or solvate thereof, wherein R is optionally substituted C3-C7 heterocyclyl. Another embodiment provides the compound, or a pharmaceutically acceptable salt or solvate thereof, wherein R is optionally substituted C3-C7 heterocyclylalkyl. Another embodiment provides the compound, or a pharmaceutically acceptable salt or solvate thereof, wherein R is —CO₂R⁵. Another embodiment provides the compound, or a pharmaceutically acceptable salt or solvate thereof, wherein R is —CONHR⁵ or —CON(R⁵)₂. Another embodiment provides the compound, or a pharmaceutically acceptable salt or solvate thereof, wherein the optionally substituted C1-C6 alkyl is a C1-C3 alkyl substituted with a C1-C3 alkoxy.

One embodiment provides a compound, or pharmaceutically acceptable salt or solvate thereof, having the structure of Formula (II):

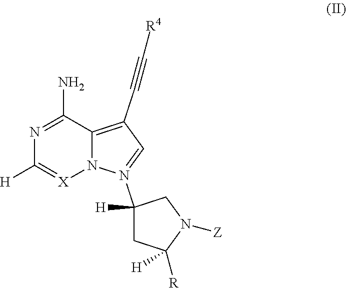

wherein,
X is C—H or N;
Z is selected from a group having the structure:

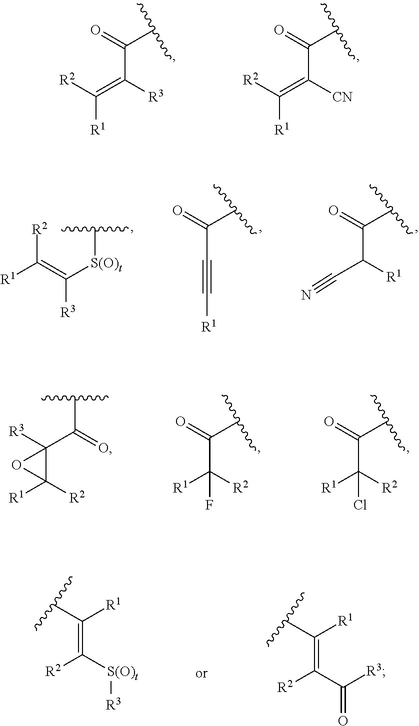

t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

One embodiment provides a compound of Formula (II), or a pharmaceutically acceptable salt or solvate thereof, wherein X is C—H.

One embodiment provides a compound of Formula (II), or a pharmaceutically acceptable salt or solvate thereof, wherein X is N.

One embodiment provides a compound of Formula (II), or a pharmaceutically acceptable salt or solvate thereof, wherein Z is

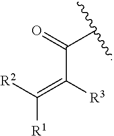

Another embodiment provides the compound, or pharmaceutically acceptable salt or solvate thereof, wherein R² is hydrogen. Another embodiment provides the compound, or pharmaceutically acceptable salt or solvate thereof, wherein R³ is hydrogen or fluoro. Another embodiment provides the compound, or pharmaceutically acceptable salt or solvate thereof, wherein R² and R³ are hydrogen. Another embodiment provides the compound, or pharmaceutically acceptable salt or solvate thereof, wherein R¹ is hydrogen. Another embodiment provides the compound, or pharmaceutically acceptable salt or solvate thereof, wherein R¹ is optionally substituted C1-C4 alkyl. Another embodiment provides the compound, or pharmaceutically acceptable salt or solvate thereof, wherein R¹ is optionally substituted C1-C2 alkyl. Another embodiment provides the compound, or pharmaceutically acceptable salt or solvate thereof, wherein R¹ is optionally substituted C1 alkyl. Another embodiment provides the compound, or pharmaceutically acceptable salt or solvate thereof, wherein the optionally substituted alkyl is substituted with an optionally substituted amino group. Another embodiment provides the compound, or pharmaceutically acceptable salt or solvate thereof, wherein the optionally substituted amino group is a dimethylamino.

One embodiment provides a compound of Formula (II), or a pharmaceutically acceptable salt or solvate thereof, wherein R⁴ is selected from an optionally substituted nitrogen-containing 9 or 10-atom heteroaryl is selected from optionally substituted benzimidazole, optionally substituted 1H-indazole, optionally substituted 2H-indazole, optionally substituted benzotriazole, optionally substituted benzoxazole, optionally substituted imidazo[4,5-c]pyridine, or optionally substituted imidazo[4,5-b]pyridine. One embodiment provides a compound of Formula (II), or a pharmaceutically acceptable salt or solvate thereof, wherein R⁴ is selected from an optionally substituted nitrogen-containing 9 or 10-atom heteroaryl is selected from quinoline, quinoxaline, pyrazolo[1,5-a]pyrimidine, imidazo[1,2-a]pyridine, pyrazolo[1,5-a]pyridine, imidazo[1,2-a]pyrimidine, imidazo[1,2-b]pyridazine, or pyrazolo[1,5-a]pyridine. Another embodiment provides the compound, or a pharmaceutically acceptable salt or solvate thereof, wherein R⁴ is an optionally substituted benzimidazole. Another embodiment provides the compound, or a pharmaceutically acceptable salt or solvate thereof, wherein R⁴ is an optionally substituted 1H-indazole. Another embodiment provides the compound, or a pharmaceutically acceptable salt or solvate thereof, wherein R⁴ is an optionally substituted 2H-indazole. Another embodiment provides the compound, or a pharmaceutically acceptable salt or solvate thereof, wherein R⁴ is an optionally substituted benzoxazole, optionally substituted imidazo[4,5-c]pyridine, or optionally substituted imidazo[4,5-b]pyridine. Another embodiment provides the compound, or a pharmaceutically acceptable salt or solvate thereof, wherein optionally substituted nitrogen-containing 9 or 10-atom heteroaryl is optionally substituted with alkyl, cycloalkyl, or halogen. Another embodiment provides the compound, or a pharmaceutically acceptable salt or solvate thereof, wherein the optionally substituted benzimidazole is optionally substituted with alkyl, cycloalkyl, or halogen. Another embodiment provides the compound, or a pharmaceutically acceptable salt or solvate thereof, wherein the optionally substituted 1H-indazole is optionally substituted with alkyl, cycloalkyl, or halogen. Another embodiment provides the compound, or a pharmaceutically acceptable salt or solvate thereof, wherein the optionally substituted 2H-indazole is optionally substituted with alkyl, cycloalkyl, or halogen.

One embodiment provides a compound of Formula (II), or a pharmaceutically acceptable salt or solvate thereof, wherein R is hydrogen. Another embodiment provides the compound, or a pharmaceutically acceptable salt or solvate thereof, wherein R is optionally substituted C1-C6 alkyl. Another embodiment provides the compound, or a pharmaceutically acceptable salt or solvate thereof, wherein R is optionally substituted C3-C7 carbocyclyl. Another embodiment provides the compound, or a pharmaceutically acceptable salt or solvate thereof, wherein R is optionally substituted C3-C7 carbocyclylalkyl. Another embodiment provides the compound, or a pharmaceutically acceptable salt or solvate thereof, wherein R is optionally substituted C3-C7 heterocyclyl. Another embodiment provides the compound, or a pharmaceutically acceptable salt or solvate thereof, wherein R is optionally substituted C3-C7 heterocyclylalkyl. Another embodiment provides the compound, or a pharmaceutically acceptable salt or solvate thereof, wherein R is —CO₂R⁵. Another embodiment provides the compound, or a pharmaceutically acceptable salt or solvate thereof, wherein R is —CONHR⁵ or —CON(R⁵)₂. Another embodiment provides the compound, or a pharmaceutically acceptable salt or solvate thereof, wherein the optionally substituted C1-C6 alkyl is a C1-C3 alkyl substituted with a C1-C3 alkoxy.

In some embodiments, the heteroaromatic FGFR kinase inhibitory compound disclosed herein has a structure provided in Table 1.

TABLE 1

| Synthetic | | |
|---|---|---|
| Chemistry | | |
| Example | Compound Structure | Compound Name |
| 1 | 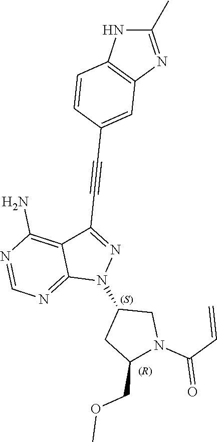 | 1-((2R,4S)-4-(4-amino-3-((2- methyl-1H-benzo[d]imidazol-5- yl)ethynyl)-1H-pyrazolo[3,4- d]pyrimidin-1-yl)-2- (methoxymethyl)pyrrolidin-1- yl)prop-2-en-1-one |
| 2 | 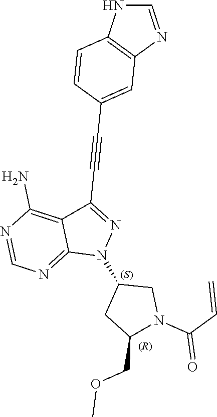 | 1-((2R,4S)-4-(3-((1H-indazol-5- yl)ethynyl)-4-amino-1H- pyrazolo[3,4-d]pyrimidin-1-yl)- 2-(methoxymethyl)pyrrolidin-1- yl)prop-2-en-1-one |
| 3 | 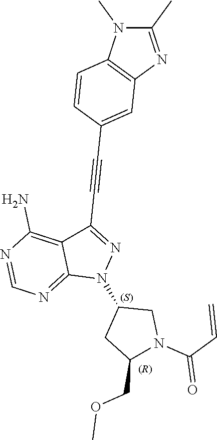 | 1-((2R,4S)-4-(4-amino-3-((1,2- dimethyl-1H-benzo[d]imidazol- 5-yl)ethynyl)-1H-pyrazolo[3,4- d]pyrimidin-1-yl)-2- (methoxymethyl)pyrrolidin-1- yl)prop-2-en-1-one |
| 4 | 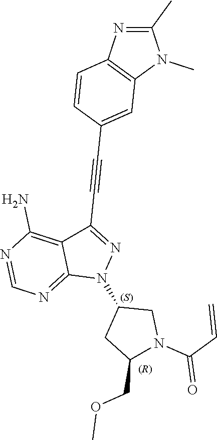 | 1-((2R,4S)-4-(4-amino-3-((1,2- dimethyl-1H-benzo[d]imidazol- 6-yl)ethynyl)-1H-pyrazolo[3,4- d]pyrimidin-1-yl)-2- (methoxymethyl)pyrrolidin-1- yl)prop-2-en-1-one |
| 5 | 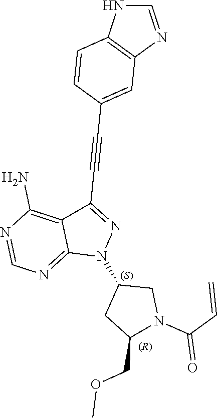 | 1-((2R,4S)-4-(3-((1H- benzo[d]imidazol-5-yl)ethynyl)- 4-amino-1H-pyrazolo[3,4- d]pyrimidin-1-yl)-2- (methoxymethyl)pyrrolidin-1- yl)prop-2-en-1-one |
| 6 | 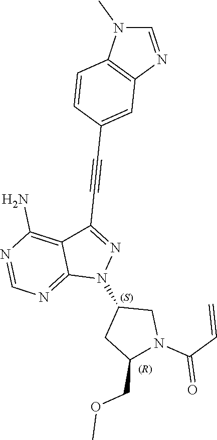 | 1-((2R,4S)-4-(4-amino-3-((1- methyl-1H-benzo[d]imidazol-5- yl)ethynyl)-1H-pyrazolo[3,4- d]pyrimidin-1-yl)-2- (methoxymethyl)pyrrolidin-1- yl)prop-2-en-1-one |
| 7 | 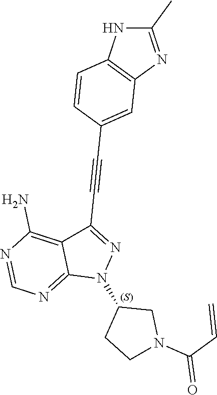 | (S)-1-(3-(4-amino-3-((2-methyl- 1H-benzo[d]imidazol-5- yl)ethynyl)-1H-pyrazolo[3,4- d]pyrimidin-1-yl)pyrrolidin-1- yl)prop-2-en-1-one |
| 8 | 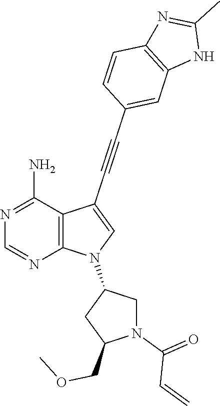 [SMILES: C=CC(=O)N1C[C@@H](n2cc(C#Cc3ccc4nc(C)[nH]c4c3)c3c(N)ncnc32)C[C@@H]1COC] | 1-[(2R,4S)-4-[4-amino-5-[2-(2- methyl-3H-1,3-benzodiazol-5- yl)ethynyl]pyrrolo[2,3- d]pyrimidin-7-yl]-2- (methoxymethyl)pyrrolidin-1- yl]prop-2-en-1-one |
| 9 | 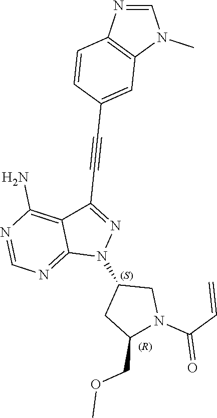 | 1-((2R,4S)-4-(4-amino-3-((1- methyl-1H-benzo[d]imidazol-6- yl)ethynyl)-1H-pyrazolo[3,4- d]pyrimidin-1-yl)-2- (methoxymethyl)pyrrolidin-1- yl)prop-2-en-1-one |
| 10 | 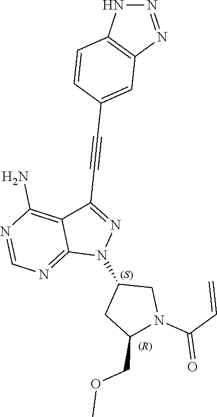 | 1-((2R,4S)-4-(3-((1H- benzo[d][1,2,3]triazol-5- yl)ethynyl)-4-amino-1H- pyrazolo[3,4-d]pyrimidin-1-yl)- 2-(methoxymethyl)pyrrolidin-1- yl)prop-2-en-1-one |
| 11 | 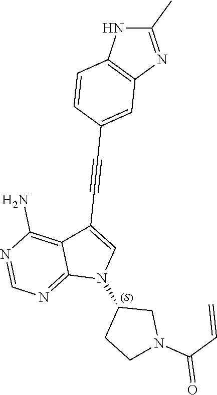 | (S)-1-(3-(4-amino-5-((2-methyl- 1H-benzo[d]imidazol-5- yl)ethynyl)-7H-pyrrolo[2,3- d]pyrimidin-7-yl)pyrrolidin-1- yl)prop-2-en-1-one |
| 12 | 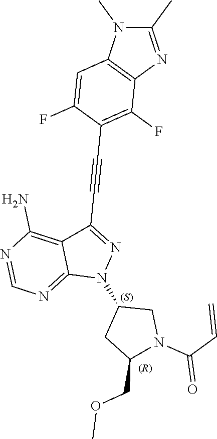 | 1-((2R,4S)-4-(4-amino-3-((4,6- difluoro-1,2-dimethyl-1H- benzo[d]imidazol-5-yl)ethynyl)- 1H-pyrazolo[3,4-d]pyrimidin-1- yl)-2- (methoxymethyl)pyrrolidin-1- yl)prop-2-en-1-one |
| 13 | 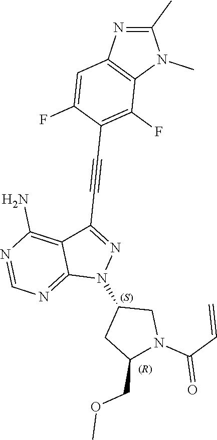 | 1-((2R,4S)-4-(4-amino-3-((5,7- difluoro-1,2-dimethyl-1H- benzo[d]imidazol-6-yl)ethynyl)- 1H-pyrazolo[3,4-d]pyrimidin-1- yl)-2- (methoxymethyl)pyrrolidin-1- yl)prop-2-en-1-one |
| 14 | 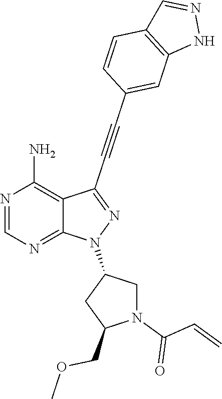 [SMILES: C=CC(=O)N1C[C@@H](n2nc(C#Cc3ccc4cn[nH]c4c3)c3c(N)ncnc32)C[C@@H]1COC] | 1-((2R,4S)-4-(3-((1H-indazol-6- yl)ethynyl)-4-amino-1H- pyrazolo[3,4-d]pyrimidin-1-yl)- 2-(methoxymethyl)pyrrolidin-1- yl)prop-2-en-1-one |
| 15 | 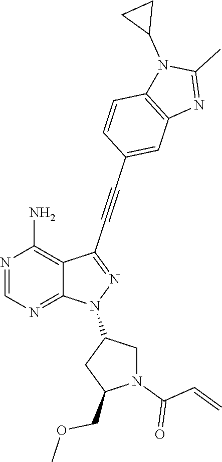 | 1-((2R,4S)-4-(4-amino-3-((1- cyclopropyl-2-methyl-1H- benzo[d]imidazol-5-yl)ethynyl)- 1H-pyrazolo[3,4-d]pyrimidin-1- yl)-2- (methoxymethyl)pyrrolidin-1- yl)prop-2-en-1-one |
| 16 | 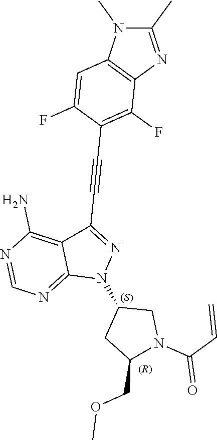 | 1-((2R,4S)-4-(4-amino-3-((4,6- difluoro-1,2-dimethyl-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 17 | 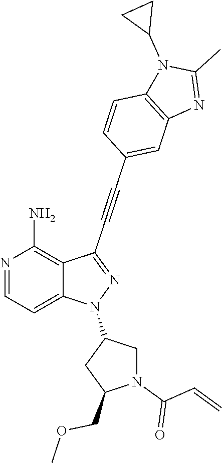 | 1-((2R,4S)-4-(4-amino-3-((1- cyclopropyl-2-methyl-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 18 | 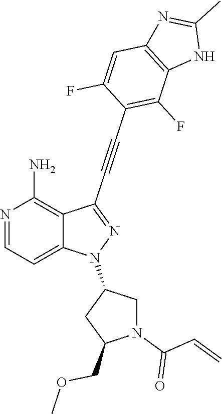 [SMILES: C=CC(=O)N1C[C@@H](n2nc(C#Cc3c(F)cc4nc(C)[nH]c4c3F)c3c(N)nccc32)C[C@@H]1COC] | 1-((2R,4S)-4-(4-amino-3-((5,7- difluoro-2-methyl-1H- benzo[d]imidazol-6-yl)ethynyl)- 1H-pyrazolo[4,3-c]pyridin-1-yl)- 2-(methoxymethyl)pyrrolidin-1- yl)prop-2-en-1-one |
| 19 | 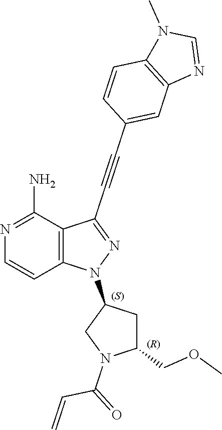 | 1-((2R,4S)-4-(4-amino-3-((1- methyl-1H-benzo[d]imidazol-5- yl)ethynyl)-1H-pyrazolo[4,3- c]pyridin-1-yl)-2- (methoxymethyl)pyrrolidin-1- yl)prop-2-en-1-one |
| 20 | 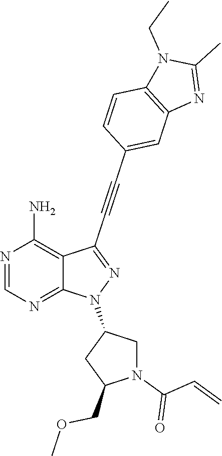 [SMILES: C=CC(=O)N1C[C@@H](n2nc(C#Cc3ccc4c(c3)nc(C)n4CC)c3c(N)ncnc32)C[C@@H]1COC] | 1-((2R,4S)-4-(4-amino-3-((1- ethyl-2-methyl-1H- benzo[d]imidazol-5-yl)ethynyl)- 1H-pyrazolo[3,4-d]pyrimidin-1- yl)-2- (methoxymethyl)pyrrolidin-1- yl)prop-2-en-1-one |
| 21 | 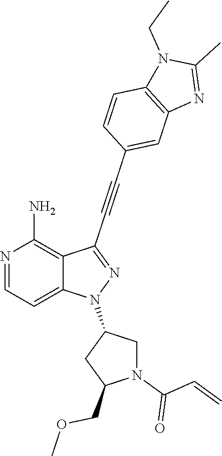 [SMILES: C=CC(=O)N1C[C@@H](n2nc(C#Cc3ccc4c(c3)nc(C)n4CC)c3c(N)nccc32)C[C@@H]1COC] | 1-((2R,4S)-4-(4-amino-3-((1- ethyl-2-methyl-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 22 | 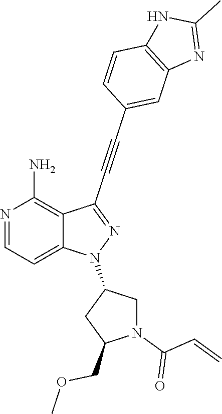 [SMILES: C=CC(=O)N1C[C@@H](n2nc(C#Cc3ccc4[nH]c(C)nc4c3)c3c(N)nccc32)C[C@@H]1COC] | 1-((2R,4S)-4-(4-amino-3-((2- methyl-1H-benzo[d]imidazol-5- yl)ethynyl)-1H-pyrazolo[4,3- c]pyridin-1-yl)-2- (methoxymethyl)pyrrolidin-1- yl)prop-2-en-1-one |
| 23 | 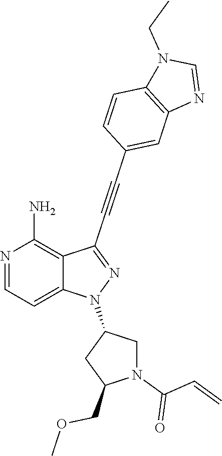 [SMILES: C=CC(=O)N1C[C@@H](n2nc(C#Cc3ccc4c(c3)ncn4CC)c3c(N)nccc32)C[C@@H]1COC] | 1-((2R,4S)-4-(4-amino-3-((1- ethyl-1H-benzo[d]imidazol-5- yl)ethynyl)-1H-pyrazolo[4,3- c]pyridin-1-yl)-2- (methoxymethyl)pyrrolidin-1- yl)prop-2-en-1-one |
| 24 | 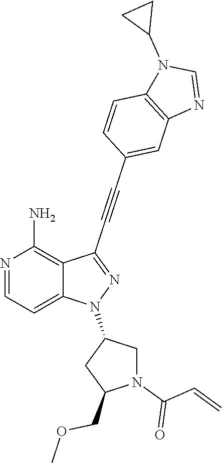 [SMILES: C=CC(=O)N1C[C@@H](n2nc(C#Cc3ccc4c(c3)ncn4C3CC3)c3c(N)nccc32)C[C@@H]1COC] | 1-((2R,4S)-4-(4-amino-3-((1- cyclopropyl-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 25 | 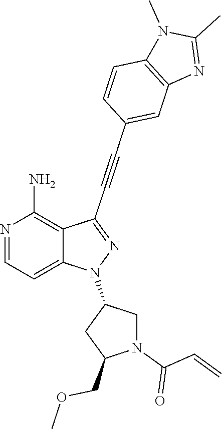 [SMILES: C=CC(=O)N1C[C@@H](n2nc(C#Cc3ccc4c(c3)nc(C)n4C)c3c(N)nccc32)C[C@@H]1COC] | 1-((2R,4S)-4-(4-amino-3-((1,2- dimethyl-1H-benzo[d]imidazol- 5-yl)ethynyl)-1H-pyrazolo[4,3- c]pyridin-1-yl)-2- (methoxymethyl)pyrrolidin-1- yl)prop-2-en-1-one |
| 26 | 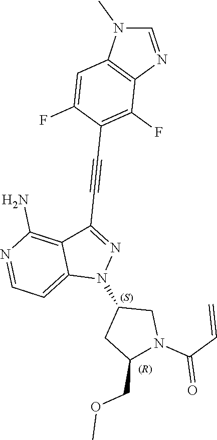 | 1-((2R,4S)-4-(4-amino-3-((4,6- difluoro-1-methyl-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 27 | 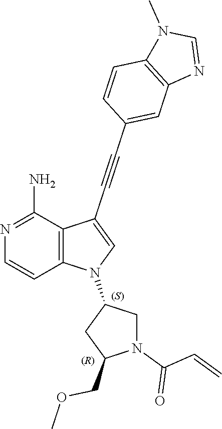 | 1-((2R,4S)-4-(4-amino-3-((1- methyl-1H-benzo[d]imidazol-5- yl)ethynyl)-1H-pyrrolo[3,2- c]pyridin-1-yl)-2- (methoxymethyl)pyrrolidin-1- yl)prop-2-en-1-one |
| 28 | 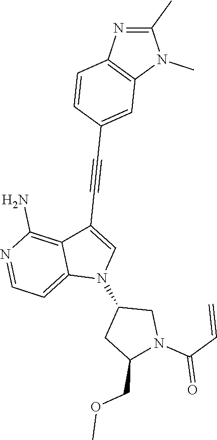 [SMILES: C=CC(=O)N1C[C@@H](n2cc(C#Cc3ccc4nc(C)n(C)c4c3)c3c(N)nccc32)C[C@@H]1COC] | 1-((2R,4S)-4-(4-amino-3-((1,2- dimethyl-1H-benzo[d]imidazol- 6-yl)ethynyl)-1H-pyrrolo[3,2- c]pyridin-1-yl)-2- (methoxymethyl)pyrrolidin-1- yl)prop-2-en-1-one |
| 29 | 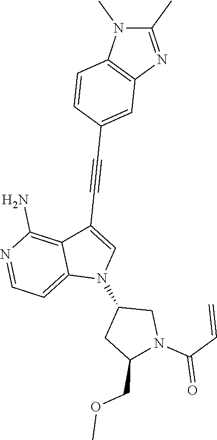 [SMILES: C=CC(=O)N1C[C@@H](n2cc(C#Cc3ccc4c(c3)nc(C)n4C)c3c(N)nccc32)C[C@@H]1COC] | 1-((2R,4S)-4-(4-amino-3-((1,2- dimethyl-1H-benzo[d]imidazol- 5-yl)ethynyl)-1H-pyrrolo[3,2- c]pyridin-1-yl)-2- (methoxymethyl)pyrrolidin-1- yl)prop-2-en-1-one |
| 30 | 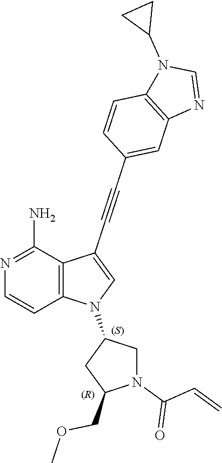 | 1-((2R,4S)-4-(4-amino-3-((1- cyclopropyl-1H- benzo[d]imidazol-5-yl)ethynyl)- 1H-pyrrolo[3,2-c]pyridin-1-yl)- 2-(methoxymethyl)pyrrolidin-1- yl)prop-2-en-1-one |
| 31 | 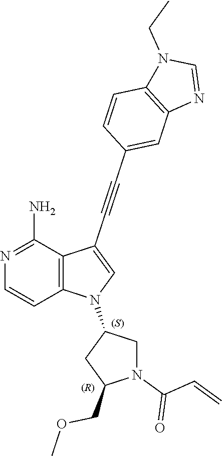 | 1-((2R,4S)-4-(4-amino-3-((1- ethyl-1H-benzo[d]imidazol-5- yl)ethynyl)-1H-pyrrolo[3,2- c]pyridin-1-yl)-2- (methoxymethyl)pyrrolidin-1- yl)prop-2-en-1-one |
| 32 | 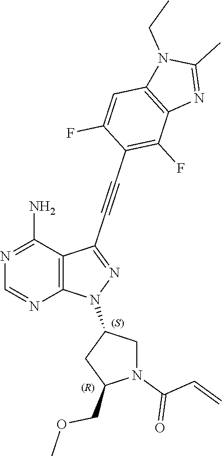 | 1-((2R,4S)-4-(4-amino-3-((1- ethyl-4,6-difluoro-2-methyl-1H- benzo[d]imidazol-5-yl)ethynyl)- 1H-pyrazolo[3,4-d]pyrimidin-1- yl)-2- (methoxymethyl)pyrrolidin-1- yl)prop-2-en-1-one |
| 33 | 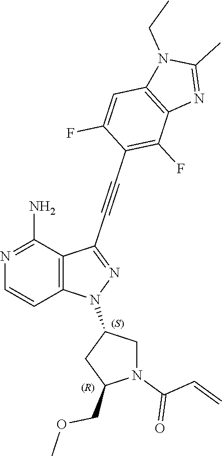 | 1-((2R,4S)-4-(4-amino-3-((1- ethyl-4,6-difluoro-2-methyl-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 34 | 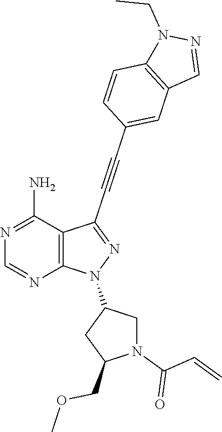 | 1-((2R,4S)-4-(4-amino-3-((1- ethyl-1H-indazol-5-yl)ethynyl)- 1H-pyrazolo[3,4-d]pyrimidin-1- yl)-2- (methoxymethyl)pyrrolidin-1- yl)prop-2-en-1-one |
| 35 | 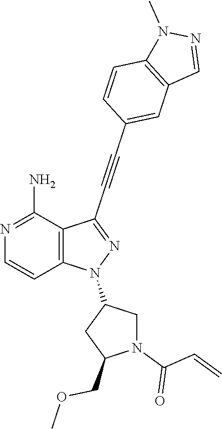 | 1-[(2R,4S)-4-[4-amino-3-[2-(1- methylindazol-5- yl)ethynyl]pyrazolo[4,3- c]pyridin-1-yl]-2- (methoxymethyl)pyrrolidin-1- yl]prop-2-en-1-one |
| 36 | 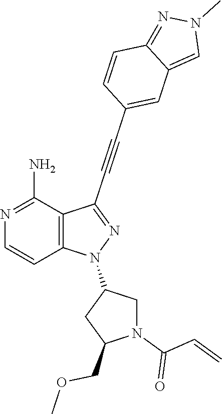 | 1-[(2R,4S)-4-[4-amino-3-[2-(2- methylindazol-5- yl)ethynyl]pyrazolo[4,3- c]pyridin-1-yl]-2- (methoxymethyl)pyrrolidin-1- yl]prop-2-en-1-one |
| 37 | 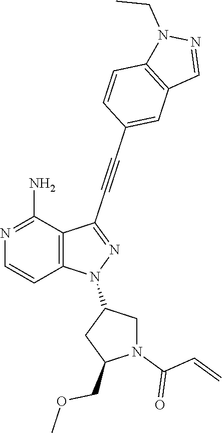 | 1-((2R,4S)-4-(4-amino-3-((1- ethyl-1H-indazol-5-yl)ethynyl)- 1H-pyrazolo[4,3-c]pyridin-1-yl)- 2-(methoxymethyl)pyrrolidin-1- yl)prop-2-en-1-one |
| 38 | 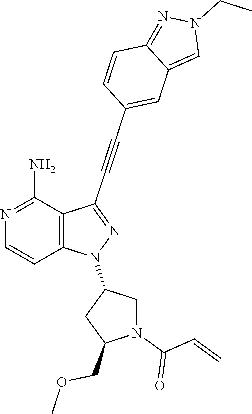 [SMILES: C=CC(=O)N1C[C@@H](n2nc(C#Cc3ccc4nn(CC)cc4c3)c3c(N)nccc32)C[C@@H]1COC] | 1-[(2R,4S)-4-[4-amino-3-[2-(2- ethylindazol-5- yl)ethynyl]pyrazolo[4,3- c]pyridin-1-yl]-2- (methoxymethyl)pyrrolidin-1- yl]prop-2-en-1-one |
| 39 | 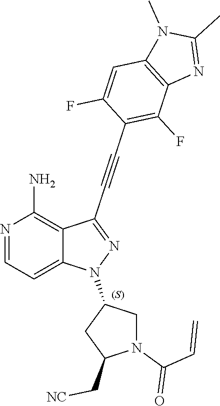 | 2-((2R,4S)-1-acryloyl-4-(4- amino-3-((4,6-difluoro-1,2- dimethyl-1H-benzo[d]imidazol- 5-yl)ethynyl)-1H-pyrazolo[4,3- c]pyridin-1-yl)pyrrolidin-2- yl)acetonitrile |
| 40 | 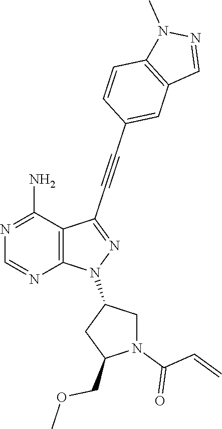 [SMILES: C=CC(=O)N1C[C@@H](n2nc(C#Cc3ccc4c(cnn4C)c3)c3c(N)ncnc32)C[C@@H]1COC] | 1-((2R,4S)-4-(4-amino-3-((1- methyl-1H-indazol-5- yl)ethynyl)-1H-pyrazolo[3,4- d]pyrimidin-1-yl)-2- (methoxymethyl)pyrrolidin-1- yl)prop-2-en-1-one |
| 41 | 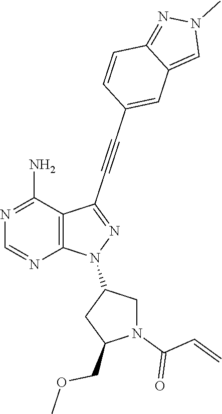 | 1-[(2R,4S)-4-[4-amino-3-[2-(2- methylindazol-5- yl)ethynyl]pyrazolo[3,4- d]pyrimidin-1-yl]-2- (methoxymethyl)pyrrolidin-1- yl]prop-2-en-1-one |
| 42 | 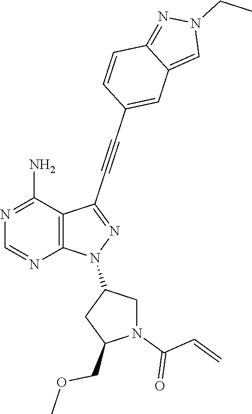 | 1-[(2R,4S)-4-[4-amino-3-[2-(2- ethylindazol-5- yl)ethynyl]pyrazolo[3,4- d]pyrimidin-1-yl]-2- (methoxymethyl)pyrrolidin-1- yl]prop-2-en-1-one |
| 43 | 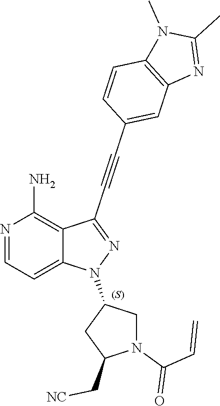 | 2-((2R,4S)-1-acryloyl-4-(4- amino-3-((1,2-dimethyl-1H- benzo[d]imidazol-5-yl)ethynyl)- 1H-pyrazolo[4,3-c]pyridin-1- yl)pyrrolidin-2-yl)acetonitrile |
| 44 | 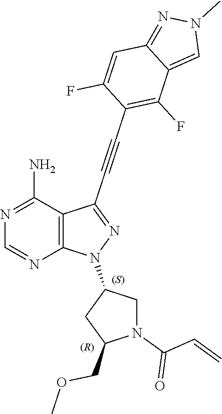 | 1-((2R,4S)-4-(4-amino-3-((4,6- difluoro-2-methyl-2H-indazol-5- yl)ethynyl)-1H-pyrazolo[3,4- d]pyrimidin-1-yl)-2- (methoxymethyl)pyrrolidin-1- yl)prop-2-en-1-one |
| 45 | 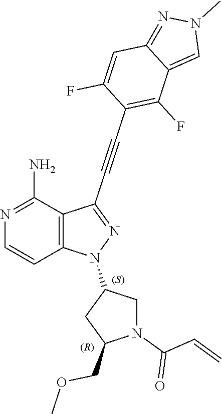 | 1-((2R,4S)-4-(4-amino-3-((4,6- difluoro-2-methyl-2H-indazol-5- yl)ethynyl)-1H-pyrazolo[4,3- c]pyridin-1-yl)-2- (methoxymethyl)pyrrolidin-1- yl)prop-2-en-1-one |
| 46 | 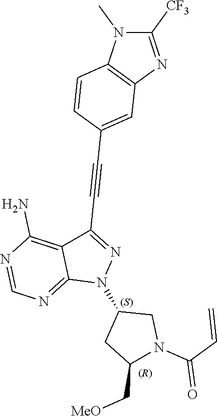 | 1-((2R,4S)-4-(4-amino-3-((1- methyl-2-(trifluoromethyl)-1H- benzo[d]imidazol-5-yl)ethynyl)- 1H-pyrazolo[3,4-d]pyrimidin-1- yl)-2- (methoxymethyl)pyrrolidin-1- yl)prop-2-en-1-one |
| 47 | 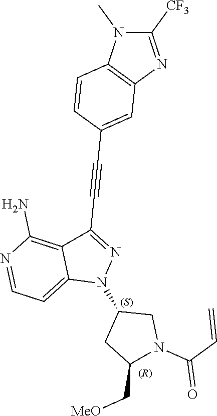 | 1-((2R,4S)-4-(4-amino-3-((1- methyl-2-(trifluoromethyl)-1H- benzo[d]imidazol-5-yl)ethynyl)- 1H-pyrazolo[4,3-c]pyridin-1-yl)- 2-(methoxymethyl)pyrrolidin-1- yl)prop-2-en-1-one |
| 48 | 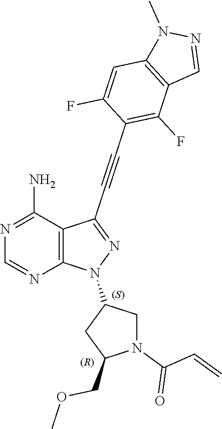 | 1-((2R,4S)-4-(4-amino-3-((4,6- difluoro-1-methyl-1H-indazol-5- yl)ethynyl)-1H-pyrazolo[3,4- d]pyrimidin-1-yl)-2- (methoxymethyl)pyrrolidin-1- yl)prop-2-en-1-one |
| 49 | 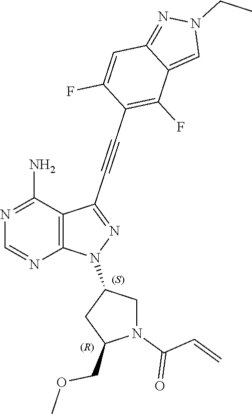 | 1-((2R,4S)-4-(4-amino-3-((2- ethyl-4,6-difluoro-2H-indazol-5- yl)ethynyl)-1H-pyrazolo[3,4- d]pyrimidin-1-yl)-2- (methoxymethyl)pyrrolidin-1- yl)prop-2-en-1-one |
| 50 | 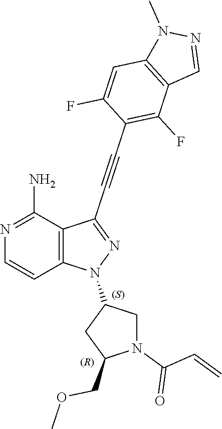 | 1-((2R,4S)-4-(4-amino-3-((4,6- difluoro-1-methyl-1H-indazol-5- yl)ethynyl)-1H-pyrazolo[4,3- c]pyridin-1-yl)-2- (methoxymethyl)pyrrolidin-1- yl)prop-2-en-1-one |
| 51 | 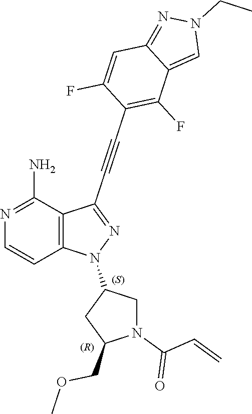 | 1-((2R,4S)-4-(4-amino-3-((2- ethyl-4,6-difluoro-2H-indazol-5- yl)ethynyl)-1H-pyrazolo[4,3- c]pyridin-1-yl)-2- (methoxymethyl)pyrrolidin-1- yl)prop-2-en-1-one |
| 52 | 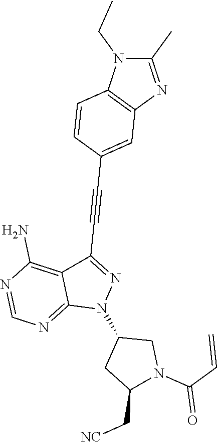 [SMILES: C=CC(=O)N1C[C@@H](n2nc(C#Cc3ccc4c(c3)nc(C)n4CC)c3c(N)ncnc32)C[C@@H]1CC#N] | 2-((2R,4S)-1-acryloyl-4-(4- amino-3-((1-ethyl-2-methyl-1H- benzo[d]imidazol-5-yl)ethynyl)- 1H-pyrazolo[3,4-d]pyrimidin-1- yl)pyrrolidin-2-yl)acetonitrile |
| 53 | 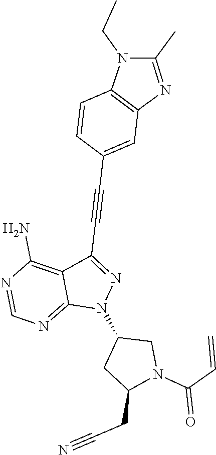 [SMILES: C=CC(=O)N1C[C@@H](n2nc(C#Cc3ccc4c(c3)nc(C)n4CC)c3c(N)ncnc32)C[C@@H]1CC#N] | 2-[(2R,4S)-4-[4-amino-3-[2-(1- ethyl-2-methyl-1,3-benzodiazol- 5-yl)ethynyl]pyrazolo[4,3- c]pyridin-1-yl]-1-(prop-2- enoyl)pyrrolidin-2- yl]acetonitrile; formic acid |
| 54 | 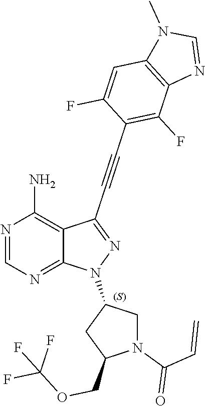 | 1-[(2R,4S)-4-[4-amino-3-[2-(4,6- difluoro-1-methyl-1,3- benzodiazol-5- yl)ethynyl]pyrazolo[3,4- d\|pyrimidin-1-yl]-2- [(trifluoromethoxy)methyl]pyrrol idin-1-yl]prop-2-en-1-one |
| 55 | 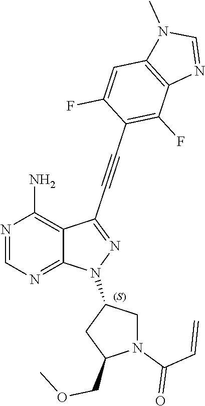 | 1-[(2R,4S)-4-[4-amino-3-[2-(4,6- difluoro-1-methyl-1,3- benzodiazol-5-yl) ethynyl] pyrazolo[3,4-d] pyrimidin-1-yl]- 2-(methoxymethyl) pyrrolidin-1- yl] prop-2-en-1-one |
| 56 | 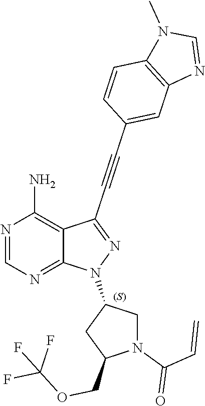 | 1-((2R,4S)-4-(4-amino-3-((1- methyl-1H-benzo[d]imidazol-5- yl)ethynyl)-1H-pyrazolo[3,4- d]pyrimidin-1-yl)-2- ((trifluoromethoxy)methyl) pyrrolidin-1-yl)prop-2-en-1-one |
| 57 | 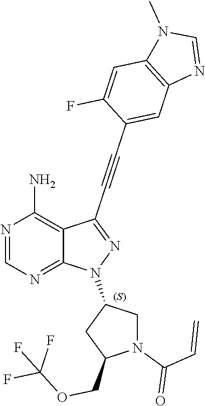 | 1-[(2R,4S)-4-[4-amino-3-[2-(6- fluoro-1-methyl-1,3- benzodiazol-5- yl)ethynyl]pyrazolo[3,4- d]pyrimidin-1-yl]-2- [(trifluoromethoxy)methyl] pyrrolidin-1-yl]prop-2-en-1-one |
| 58 | 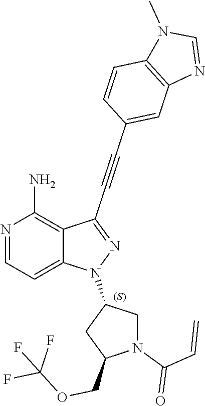 | 1-((2R,4S)-4-(4-amino-3-((1- methyl-1H-benzo[d]imidazol-5- yl)ethynyl)-1H-pyrazolo[4,3- c]pyridin-1-yl)-2- ((trifluoromethoxy)methyl) pyrrolidin-1-yl)prop-2-en-1-one formate |
| 59 | 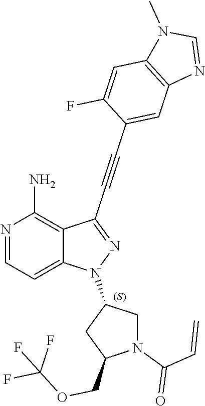 | 1-((2R,4S)-4-(4-amino-3-((6- fluoro-1-methyl-1H- benzo[d]imidazol-5-yl)ethynyl)- 1H-pyrazolo[4,3-c]pyridin-1-yl)- 2- ((trifluoromethoxy)methyl) pyrrolidin-1-yl)prop-2-en-1-one formate |
| 60 | 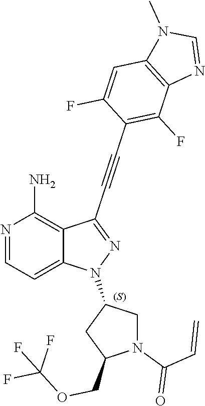 | 1-[(2R,4S)-4-[4-amino-3-[2-(4,6- difluoro-1-methyl-1,3- benzodiazol-5- yl)ethynyl]pyrazolo[4,3- c]pyridin-1-yl]-2- [(trifluoromethoxy)methyl] pyrrolidin-1-yl]prop-2-en-1-one |
| 61 | 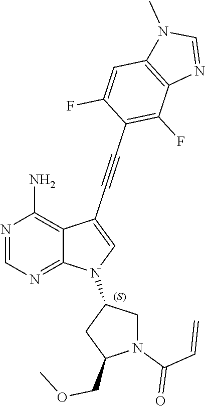 | 1-[(2R,4S)-4-[4-amino-5-[2-(4,6- difluoro-1-methyl-1,3- benzodiazol-5- yl)ethynyl]pyrrolo[2,3- d]pyrimidin-7-yl]-2- (methoxymethyl)pyrrolidin-1- yl]prop-2-en-1-one |
| 62 | 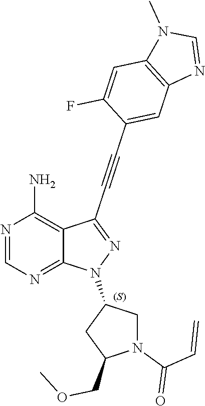 | 1-[(2R,4S)-4-[4-amino-3-[2-(6- fluoro-1-methyl-1,3- benzodiazol-5- yl)ethynyl]pyrazolo[3,4- d]pyrimidin-1-yl]-2- (methoxymethyl)pyrrolidin-1- yl]prop-2-en-1-one |
| 63 | 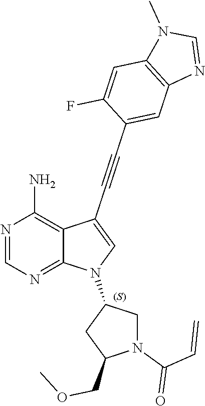 | 1-[(2R,4S)-4-[4-amino-5-[2-(6- fluoro-1-methyl-1,3- benzodiazol-5- yl)ethynyl]pyrrolo[2,3- d]pyrimidin-7-yl]-2- (methoxymethyl)pyrrolidin-1- yl]prop-2-en-1-one |
| 64 | 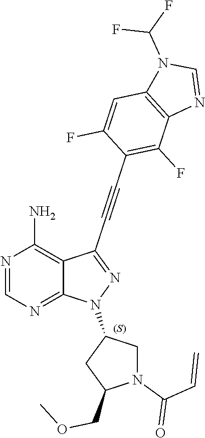 | 1-[(2R,4S)-4-(4-amino-3-[2-[1- (difluoromethyl)-4,6-difluoro- 1,3-benzodiazol-5- yl]ethynyl]pyrazolo[3,4- d]pyrimidin-1-yl)-2- (methoxymethyl)pyrrolidin-1- yl]prop-2-en-1-one |
| 65 | 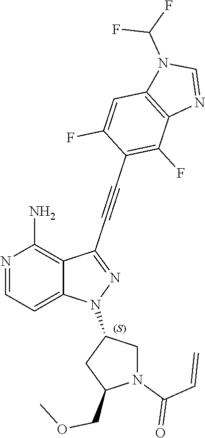 | 1-[(2R,4S)-4-(4-amino-3-[2-[1- (difluoromethyl)-4,6-difluoro- 1,3-benzodiazol-5- yl]ethynyl]pyrazolo[4,3- c]pyridin-1-yl)-2- (methoxymethyl)pyrrolidin-1- yl]prop-2-en-1-one |
| 66 | 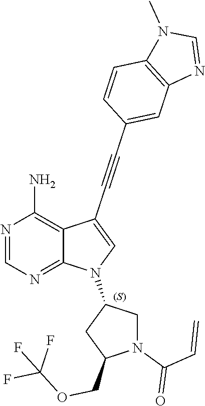 | 1-[(2R,4S)-4-[4-amino-5-[2-(1- methyl-1,3-benzodiazol-5- yl)ethynyl]pyrrolo[2,3- d]pyrimidin-7-yl]-2- [(trifluoromethoxy)methyl] pyrrolidin-1-yl]prop-2-en-1-one |
| 67 | 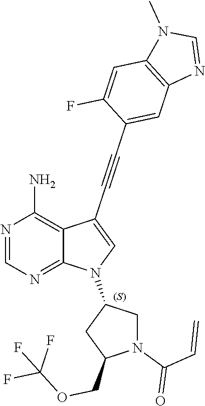 | 1-((2R,4S)-4-(4-amino-5-((6- fluoro-1-methyl-1H- benzo[d]imidazol-5-yl)ethynyl)- 7H-pyrrolo[2,3-d]pyrimidin-7- yl)-2- ((trifluoromethoxy)methyl) pyrrolidin-1-yl)prop-2-en-1-one |
| 68 | 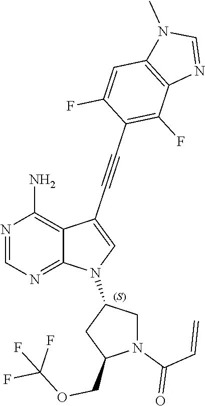 | 1-((2R,4S)-4-(4-amino-5-((4,6- difluoro-1-methyl-1H- benzo[d]imidazol-5-yl)ethynyl)- 7H-pyrrolo[2,3-d]pyrimidin-7- yl)-2- ((trifluoromethoxy)methyl) pyrrolidin-1-yl)prop-2-en-1-one |
| 69 | 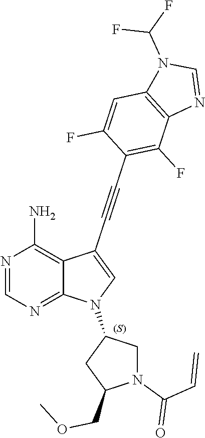 | 1-[(2R,4S)-4-(4-amino-5-[2-[1- (difluoromethyl)-4,6-difluoro- 1,3-benzodiazol-5-yl]ethynyl] pyrrolo[2,3-d]pyrimidin-7-yl)-2- (methoxymethyl)pyrrolidin-1- yl]prop-2-en-1-one |
| 70 | 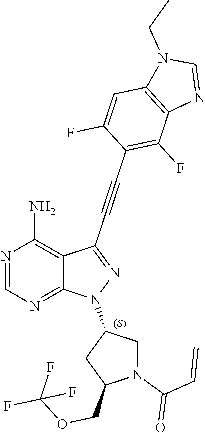 | 1-[(2R,4S)-4-[4-amino-3-[2-(1- ethyl-4,6-difluoro-1,3- benzodiazol-5- yl)ethynyl]pyrazolo[3,4- d]pyrimidin-1-yl]-2- [(trifluoromethoxy)methyl] pyrrolidin-1-yl]prop-2-en-1-one |
| 71 | 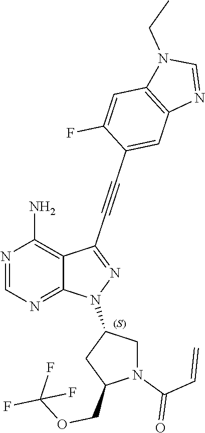 | 1-[(2R,4S)-4-[4-amino-3-[2-(1- ethyl-6-fluoro-1,3-benzodiazol- 5-yl)ethynyl]pyrazolo[3,4- d]pyrimidin-1-yl]-2- [(trifluoromethoxy)methyl] pyrrolidin-1-yl]prop-2-en-1-one |
| 72 | 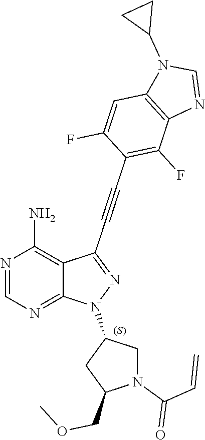 | 1-[(2R,4S)-4-[4-amino-3-[2-(1- cyclopropyl-4,6-difluoro-1,3- benzodiazol-5- yl)ethynyl]pyrazolo[3,4- d]pyrimidin-1-yl]-2- (methoxymethyl)pyrrolidin-1- yl]prop-2-en-1-one |
| 73 | 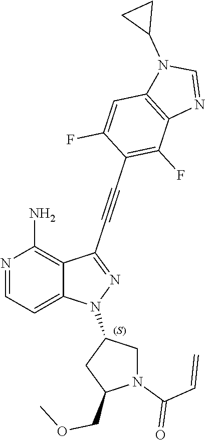 | 1-[(2R,4S)-4-[4-amino-3-[2-(1- cyclopropyl-4,6-difluoro-1,3- benzodiazol-5- yl)ethynyl]pyrazolo[4,3- c]pyridin-1-yl]-2- (methoxymethyl)pyrrolidin-1- yl]prop-2-en-1-one; formic acid |
| 74 | 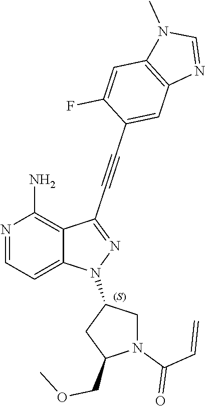 | 1-[(2R,4S)-4-[4-amino-3-[2-(6- fluoro-1-methyl-1,3- benzodiazol-5- yl)ethynyl]pyrazolo[4,3- c]pyridin-1-yl]-2- (methoxymethyl)pyrrolidin-1- yl]prop-2-en-1-one |
| 75 | 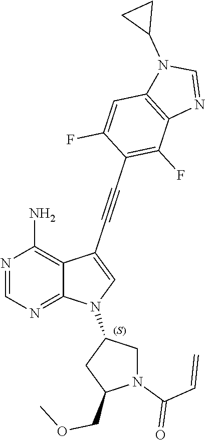 | 1-[(2R,4S)-4-[4-amino-5-[2-(1- cyclopropyl-4,6-difluoro-1,3- benzodiazol-5- yl)ethynyl]pyrrolo[2,3- d]pyrimidin-7-yl]-2- (methoxymethyl )pyrrolidin-1- yl]prop-2-en-1-one |
| 76 | 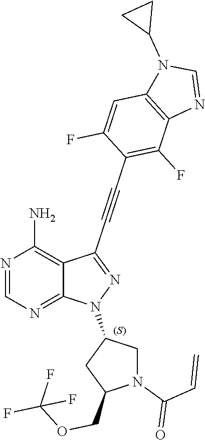 | 1-[(2R,4S)-4-[4-amino-3-[2-(1- cyclopropyl-4,6-difluoro-1,3- benzodiazol-5- yl)ethynyl]pyrazolo[3,4- d]pyrimidin-1-yl]-2- [(trifluoromethoxy)methyl] pyrrolidin-1-yl]prop-2-en-1-one |
| 77 | 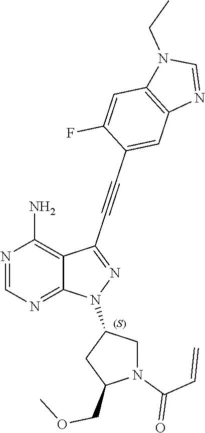 | 1-[(2R,4S)-4-[4-amino-3-[2-(1- ethyl-6-fluoro-1,3-benzodiazol- 5-yl)ethynyl]pyrazolo[3,4- d]pyrimidin-1-yl]-2- (methoxymethyl)pyrrolidin-1- yl]prop-2-en-1-one |
| 78 | 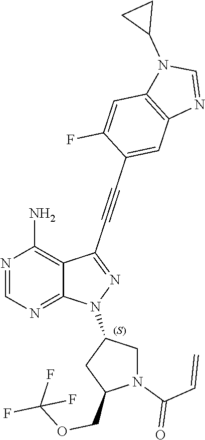 | 1-((2R,4S)-4-(4-amino-3-((1- cyclopropyl-6-fluoro-1H- benzo[d]imidazol-5-yl)ethynyl)- 1H-pyrazolo[3,4-d]pyrimidin-1- yl)-2- ((trifluoromethoxy)methyl) pyrrolidin-1-yl)prop-2-en-1-one |
| 79 | 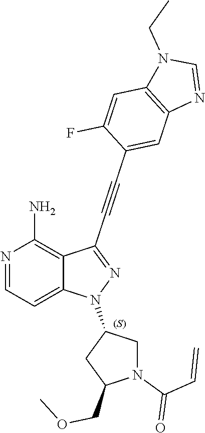 | 1-[(2R,4S)-4-[4-amino-3-[2-(1- ethyl-6-fluoro-1,3-benzodiazol- 5-yl)ethynyl]pyrazolo[4,3- c]pyridin-1-yl]-2- (methoxymethyl)pyrrolidin-1- yl]prop-2-en-1-one |
| 80 | 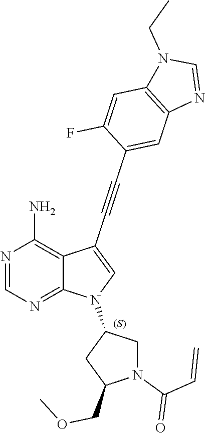 | 1-[(2R,4S)-4-[4-amino-5-[2-(1- ethyl-6-fluoro-1,3-benzodiazol- 5-yl)ethynyl]pyrrolo[2,3- d]pyrimidin-7-yl]-2- (methoxymethyl)pyrrolidin-1- yl]prop-2-en-1-one |
| 81 | 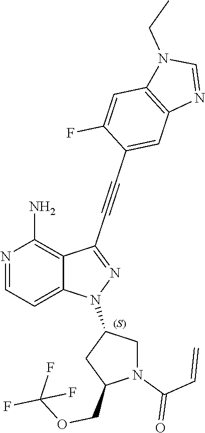 | 1-[(2R,4S)-4-[4-amino-3-[2-(1- ethyl-6-fluoro-1,3-benzodiazol- 5-yl)ethynyl]pyrazolo[4,3- c]pyridin-1-yl]-2- [(trifluoromethoxy)methyl] pyrrolidin-1-yl]prop-2-en-1-one |
| 82 | 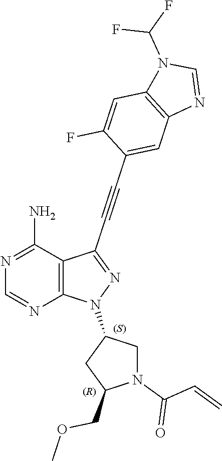 | 1-[(2R,4S)-4-(4-amino-3-[2-[1- (difluoromethyl)-6-fluoro-1,3- benzodiazol-5- yl]ethynyl]pyrazolo[3,4- d]pyrimidin-1-yl)-2- (methoxymethyl)pyrrolidin-1- yl]prop-2-en-1-one |
| 83 | 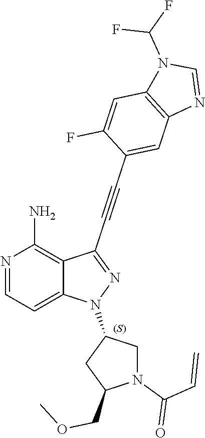 | 1-[(2R,4S)-4-(4-amino-3-[2-[1- (difluoromethyl)-6-fluoro-1,3- benzodiazol-5- yl]ethynyl]pyrazolo[4,3- c]pyridin-1-yl)-2- (methoxymethyl)pyrrolidin-1- yl]prop-2-en-1-one |
| 84 | 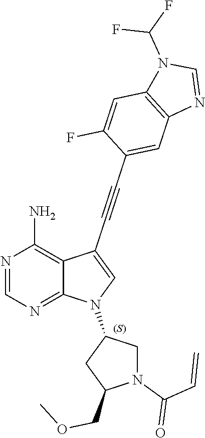 | 1-[(2R,4S)-4-(4-amino-5-[2-[1- (difluoromethyl)-6-fluoro-1,3- benzodiazol-5- yl]ethynyl]pyrrolo[2,3- d]pyrimidin-7-yl)-2- (methoxymethyl)pyrrolidin-1- yl]prop-2-en-1-one |
| 85 | 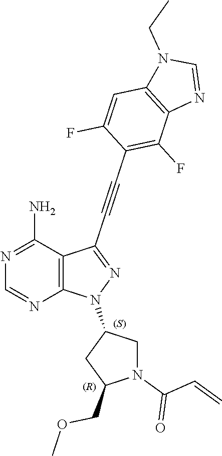 | 1-[(2R,4S)-4-[4-amino-3-[2-(1- ethyl-4,6-difluoro-1,3- benzodiazol-5- yl)ethynyl]pyrazolo[3,4- d]pyrimidin-1-yl]-2- (methoxymethyl)pyrrolidin-1- yl]prop-2-en-1-one |
| 86 | 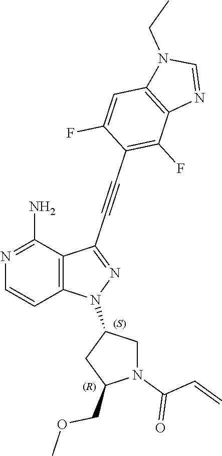 | 1-[(2R,4S)-4-[4-amino-3-[2-(1- ethyl-4,6-difluoro-1,3- benzodiazol-5- yl)ethynyl]pyrazolo[4,3- c]pyridin-1-yl]-2- (methoxymethyl)pyrrolidin-1- yl]prop-2-en-1-one |
| 87 | 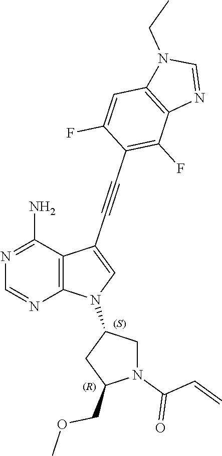 | 1-[(2R,4S)-4-[4-amino-5-[2-(1- ethyl-4,6-difluoro-1,3- benzodiazol-5- yl)ethynyl]pyrrolo[2,3- d]pyrimidin-7-yl]-2- (methoxymethyl)pyrrolidin-1- yl]prop-2-en-1-on |
| 88 | 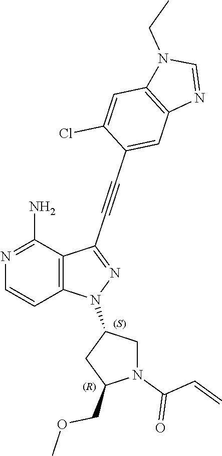 | 1-[(2R,4S)-4-[4-amino-3-[2-(6- chloro-1-ethyl-1,3-benzodiazol- 5-yl)ethynyl]pyrazolo[4,3- c]pyridin-1-yl]-2- (methoxymethyl)pyrrolidin-1- yl]prop-2-en-1-one |
| 89 | 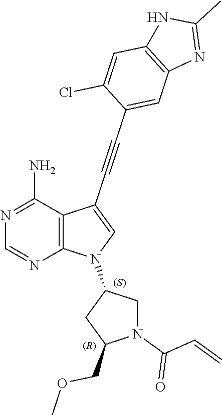 | 1-[(2R,4S)-4-[4-amino-5-[2-(6- chloro-2-methyl-1H-1,3- benzodiazol-5- yl)ethynyl]pyrrolo[2,3- d]pyrimidin-7-yl]-2- (methoxymethyl)pyrrolidin-1- yl]prop-2-en-1-one |
| 90 | 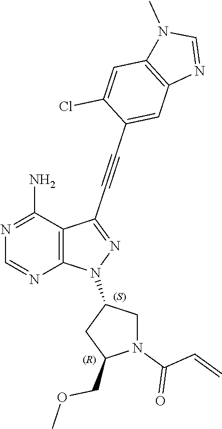 | 1-[(2R,4S)-4-[4-amino-3-[2-(6- chloro-1-methyl-1,3- benzodiazol-5- yl)ethynyl]pyrazolo[3,4- d]pyrimidin-1-yl]-2- (methoxymethyl)pyrrolidin-1- yl]prop-2-en-1-one |
| 91 | 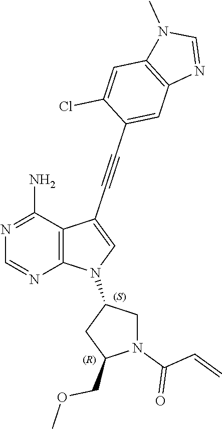 | 1-[(2R,4S)-4-[4-amino-5-[2-(6- chloro-1-methyl-1,3- benzodiazol-5- yl)ethynyl]pyrrolo[2,3- d]pyrimidin-7-yl]-2- (methoxymethyl)pyrrolidin-1- yl]prop-2-en-1-one |
| 92 | 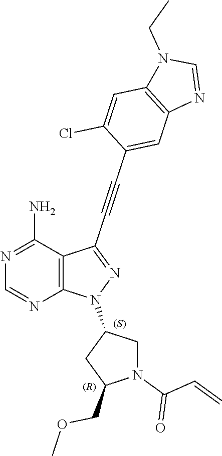 | 3-[2-(1-ethyl-4,6-difluoro-1,3- benzodiazol-5-yl)ethynyl]-5- (methylamino)-1-[(3S,5R)-1- (prop-2-enoyl)-5- [(trifluoromethoxy)methyl] pyrrolidin-3-yl]pyrazole-4- carboxamide |
| 93 | 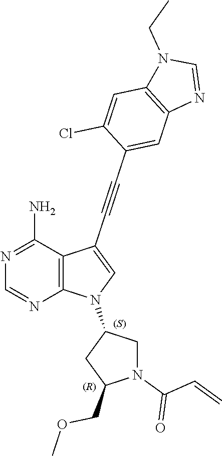 | 1-[(2R,4S)-4-[4-amino-5-[2-(6- chloro-1-ethyl-1,3-benzodiazol- 5-yl)ethynyl]pyrrolo[2,3- d]pyrimidin-7-yl]-2- (methoxymethyl)pyrrolidin-1- yl]prop-2-en-1-one |
| 94 | 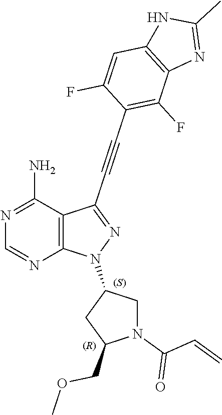 | 1-[(2R,4S)-4-[4-amino-3-[2-(4,6- difluoro-2-methyl-1H-1,3- benzodiazol-5- yl)ethynyl]pyrazolo[3,4- d]pyrimidin-1-yl]-2- (methoxymethyl)pyrrolidin-1- yl]prop-2-en-1-one |
| 95 | 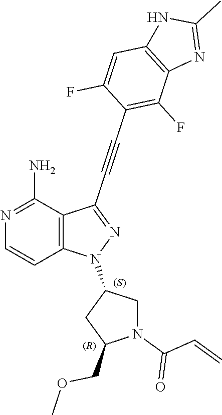 | 1-[(2R,4S)-4-[4-amino-3-[2-(4,6- difluoro-2-methyl-1H-1,3- benzodiazol-5- yl)ethynyl]pyrazolo[4,3- c]pyridin-1-yl]-2- (methoxymethyl)pyrrolidin-1- yl]prop-2-en-1-one |
| 96 | 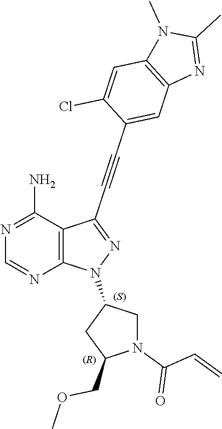 | 1-[(2R,4S)-4-[4-amino-3-[2-(6- chloro-1,2-dimethyl-1,3- benzodiazol-5- yl)ethynyl]pyrazolo[3,4- d\|pyrimidin-1-yl]-2- (methoxymethyl)pyrrolidin-1- yl]prop-2-en-1-one |
| 97 | 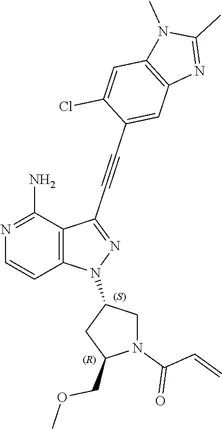 | 1-[(2R,4S)-4-[4-amino-3-[2-(6- chloro-1,2-dimethyl-1,3- benzodiazol-5- yl)ethynyl]pyrazolo[4,3- c]pyridin-1-yl]-2- (methoxymethyl)pyrrolidin-1- yl]prop-2-en-1-one |
| 98 | 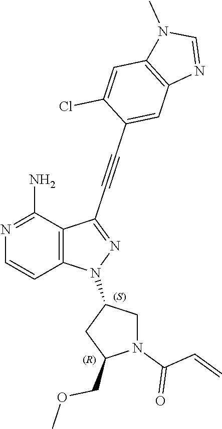 | 1-[(2R,4S)-4-[4-amino-3-[2-(6- chloro-1-methyl-1,3- benzodiazol-5- yl)ethynyl]pyrazolo[4,3- c]pyridin-1-yl]-2- (methoxymethyl)pyrrolidin-1- yl]prop-2-en-1-one |
| 99 | 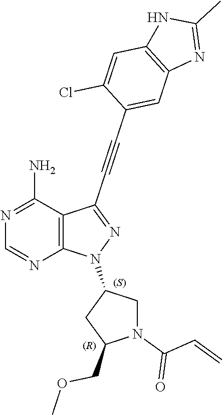 | 1-[(2R,4S)-4-[4-amino-3-[2-(6- chloro-2-methyl-1H-1,3- benzodiazol-5- yl)ethynyl]pyrazolo[3,4- d]pyrimidin-1-yl]-2- (methoxymethyl)pyrrolidin-1- yl]prop-2-en-1-one |
| 100 | 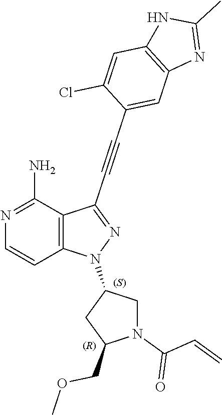 | 1-[(2R,4S)-4-[4-amino-3-[2-(6- chloro-2-methyl-1H-1,3- benzodiazol-5- yl)ethynyl]pyrazolo[4,3- c]pyridin-1-yl]-2- (methoxymethyl)pyrrolidin-1- yl]prop-2-en-1-one |
| 101 | 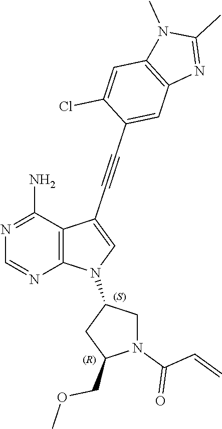 | 1-[(2R,4S)-4-[4-amino-5-[2-(6- chloro-1,2-dimethyl-1,3- benzodiazol-5- yl)ethynyl]pyrrolo[2,3- d]pyrimidin-7-yl]-2- (methoxymethyl)pyrrolidin-1- yl]prop-2-en-1-one |
| 102 | 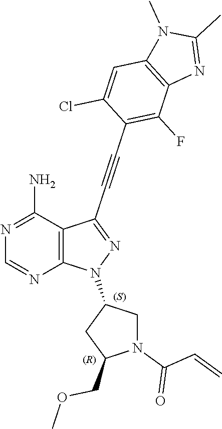 | 1-((2R,4S)-4-(4-amino-3-((6- chloro-4-fluoro-1,2-dimethyl- 1H-benzo[d]imidazol-5- yl)ethynyl)-1H-pyrazolo[3,4- d]pyrimidin-1-yl)-2- (methoxymethyl)pyrrolidin-1- yl)prop-2-en-1-one |
| 103 | 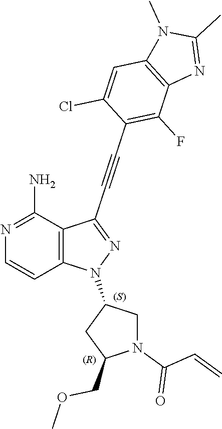 | 1-((2R,4S)-4-(4-amino-3-((6- chloro-4-fluoro-1,2-dimethyl- 1H-benzo[d]imidazol-5- yl)ethynyl)-1H-pyrazolo[4,3- c]pyridin-1-yl)-2- (methoxymethyl)pyrrolidin-1- yl)prop-2-en-1-one |
| 104 | 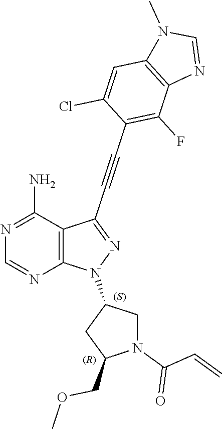 | 1-[(2R,4S)-4-[4-amino-3-[2-(6- chloro-4-fluoro-1-methyl-1,3- benzodiazol-5- yl)ethynyl]pyrazolo[3,4- d]pyrimidin-1-yl]-2- (methoxymethyl)pyrrolidin-1- yl]prop-2-en-1-one |
| 105 | 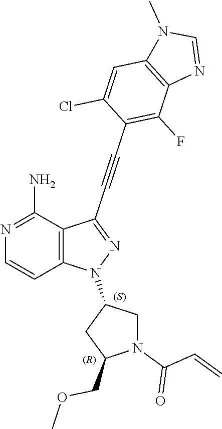 | 1-[(2R,4S)-4-[4-amino-3-[2-(6- chloro-4-fluoro-1-methyl-1,3- benzodiazol-5- yl)ethynyl]pyrazolo[4,3- c]pyridin-1-yl]-2- (methoxymethyl)pyrrolidin-1- yl]prop-2-en-1-one |
| 106 | 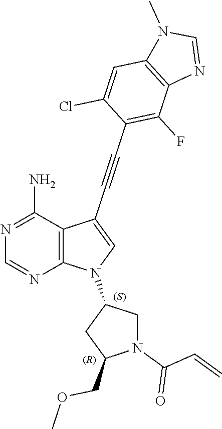 | 1-[(2R,4S)-4-[4-amino-5-[2-(6- chloro-4-fluoro-1-methyl-1,3- benzodiazol-5- yl)ethynyl]pyrrolo[2,3- d]pyrimidin-7-yl]-2- (methoxymethyl)pyrrolidin-1- yl]prop-2-en-1-one |
| 107 | 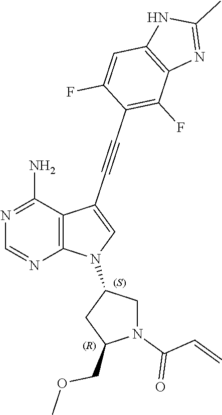 | 1-[(2R,4S)-4-[4-amino-5-[2-(4,6- difluoro-2-methyl-1H-1,3- benzodiazol-5- yl)ethynyl]pyrrolo[2,3- d]pyrimidin-7-yl]-2- (methoxymethyl)pyrrolidin-1- yl]prop-2-en-1-one |
| 108 | 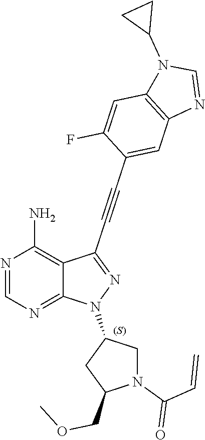 | 1-[(2R,4S)-4-[4-amino-3-[2-(1- cyclopropyl-6-fluoro-1,3- benzodiazol-5- yl)ethynyl]pyrazolo[3,4- d]pyrimidin-1-yl]-2- (methoxymethyl)pyrrolidin-1- yl]prop-2-en-1-one |
| 109 | 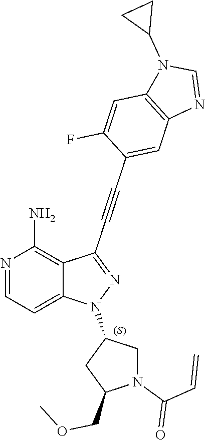 | 1-[(2R,4S)-4-[4-amino-3-[2-(1- cyclopropyl-6-fluoro-1,3- benzodiazol-5- yl)ethynyl]pyrazolo[4,3- c]pyridin-1-yl]-2- (methoxymethyl)pyrrolidin-1- yl]prop-2-en-1-one |
| 110 | 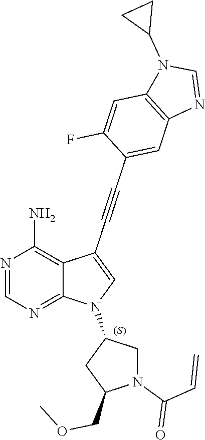 | 1-[(2R,4S)-4-[4-amino-5-[2-(1- cyclopropyl-6-fluoro-1,3- benzodiazol-5- yl)ethynyl]pyrrolo[2,3- d]pyrimidin-7-yl]-2- (methoxymethyl)pyrrolidin-1- yl]prop-2-en-1-one |
| 112 | 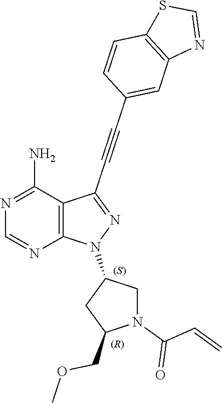 | 1-((2R,4S)-4-(4-amino-3- (benzo[d]thiazol-5-ylethynyl)- 1H-pyrazolo[3,4-d]pyrimidin-1- yl)-2- (methoxymethyl)pyrrolidin-1- yl)prop-2-en-1-one |
| 113 | 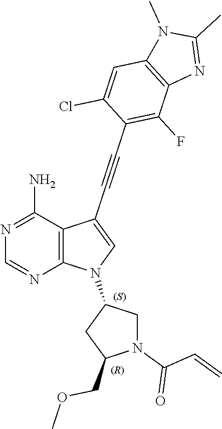 | 1-((2R,4S)-4-(4-amino-5-((6- chloro-4-fluoro-1,2-dimethyl- 1H-benzo[d]imidazol-5- yl)ethynyl)-7H-pyrrolo[2,3- d]pyrimidin-7-yl)-2- (methoxymethyl)pyrrolidin-1- yl)prop-2-en-1-one |
| 114 | 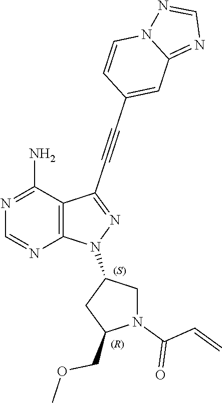 | 1-[(2R,4S)-4-[4-amino-3-(2- [[1,2,4]triazolo[1,5-a]pyridin-7- yl]ethynyl)pyrazolo[3,4- d]pyrimidin-1-yl]-2- (methoxymethyl)pyrrolidin-1- yl]prop-2-en-1-one |
| 115 | 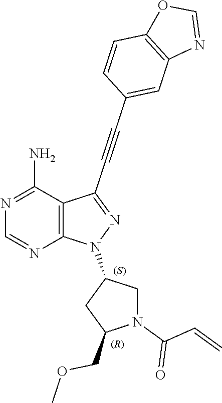 | 1-[(2R,4S)-4-[4-amino-3-[2-(1,3- benzoxazol-5- yl)ethynyl]pyrazolo[3,4- d]pyrimidin-1-yl]-2- (methoxymethyl)pyrrolidin-1- yl]prop-2-en-1-one |
| 116 | 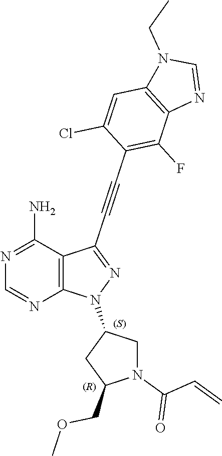 | 1-[(2R,4S)-4-[4-amino-3-[2-(6- chloro-1-ethyl-4-fluoro-1,3- benzodiazol-5- yl)ethynyl]pyrazolo[3,4- d]pyrimidin-1-yl]-2- (methoxymethyl)pyrrolidin-1- yl]prop-2-en-1-one |
| 117 | 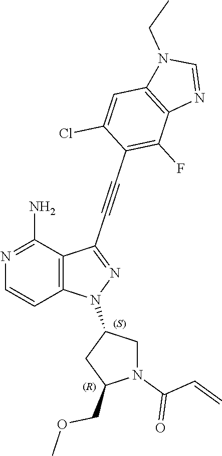 | 1-[(2R,4S)-4-[4-amino-3-[2-(6- chloro-1-ethyl-4-fluoro-1,3- benzodiazol-5- yl)ethynyl]pyrazolo[4,3- c]pyridin-1-yl]-2- (methoxymethyl)pyrrolidin-1- yl]prop-2-en-1-one |
| 118 | 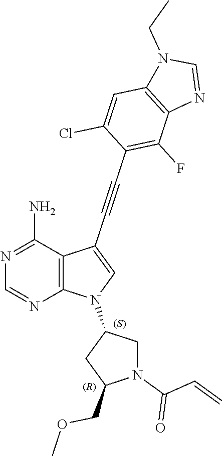 | 1-1-[(2R,4S)-4-[4-amino-5-[2-(6- chloro-1-ethyl-4-fluoro-1,3- benzodiazol-5- yl)ethynyl]pyrrolo[2,3- d]pyrimidin-7-yl]-2- (methoxymethyl)pyrrolidin-1- yl]prop-2-en-1-one |
| 119 | 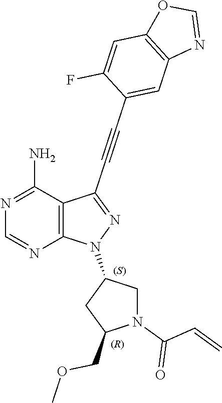 | 1-[(2R,4S)-4-[4-amino-3-[2-(6- fluoro-1,3-benzoxazol-5- yl)ethynyl]pyrazolo[3,4- d]pyrimidin-1-yl]-2- (methoxymethyl)pyrrolidin-1- yl]prop-2-en-1-one |
| 120 | 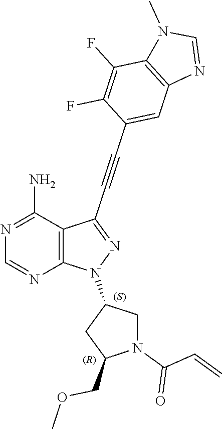 | 1-((2R,4S)-4-(4-amino-3-((6,7- difluoro-1-methyl-1H- benzo[d]imidazol-5-yl)ethynyl)- 1H-pyrazolo[3,4-d]pyrimidin-1- yl)-2- (methoxymethyl)pyrrolidin-1- yl)prop-2-en-1-one |
| 121 | 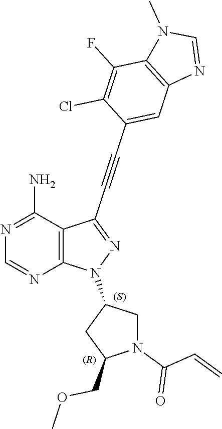 | 1-[(2R,4S)-4-[4-amino-3-[2-(6- chloro-7-fluoro-1-methyl-1,3- benzodiazol-5- yl)ethynyl]pyrazolo[3,4- d]pyrimidin-1-yl]-2- (methoxymethyl)pyrrolidin-1- yl]prop-2-en-1-one |
| 122 | 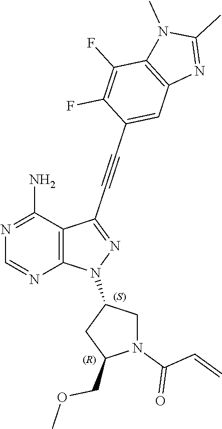 | 1-[(2R,4S)-4-[4-amino-3-[2-(6,7- difluoro-1,2-dimethyl-1,3- benzodiazol-5- yl)ethynyl]pyrazolo[3,4- d]pyrimidin-1-yl]-2- (methoxymethyl)pyrrolidin-1- yl]prop-2-en-1-one |
| 123 | 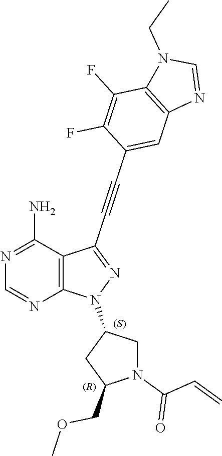 | 1-[(2R,4S)-4-[4-amino-3-[2-(1- ethyl-6,7-difluoro-1,3- benzodiazol-5- yl)ethynyl]pyrazolo[3,4- d]pyrimidin-1-yl]-2- (methoxymethyl)pyrrolidin-1- yl]prop-2-en-1-one |
| 124 | 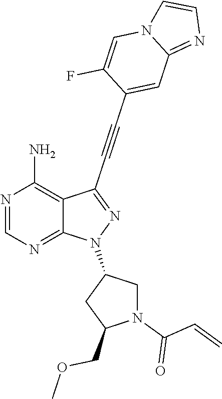 [SMILES: C=CC(=O)N1C[C@@H](n2nc(C#Cc3cc4nccn4cc3F)c3c(N)ncnc32)C[C@@H]1COC] | 1-((2R,4S)-4-(4-amino-3-((6- fluoroimidazo[1,2-a]pyridin-7- yl)ethynyl)-1H-pyrazolo[3,4- d]pyrimidin-1-yl)-2- (methoxymethyl)pyrrolidin-1- yl)prop-2-en-1-one |
| 125 | 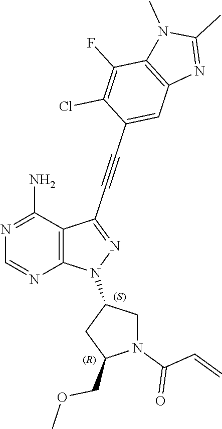 | 1-[(2R,4S)-4-[4-amino-3-[2-(6- chloro-7-fluoro-1,2-dimethyl- 1,3-benzodiazol-5- yl)ethynyl]pyrazolo[3,4- d]pyrimidin-1-yl]-2- (methoxymethyl)pyrrolidin-1- yl]prop-2-en-1-one |
| 126 | 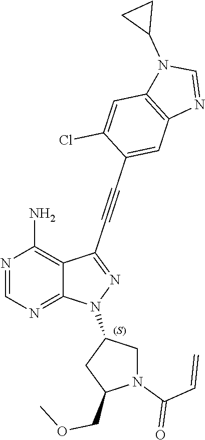 | 1-[(2R,4S)-4-[4-amino-3-[2-(6- chloro-1-cyclopropyl-1,3- benzodiazol-5- yl)ethynyl]pyrazolo[3,4- d]pyrimidin-1-yl]-2- (methoxymethyl)pyrrolidin-1- yl]prop-2-en-1-one |
| 127 | 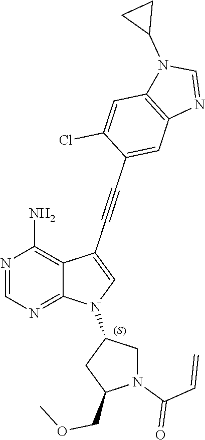 | 1-[(2R,4S)-4-[4-amino-5-[2-(6- chloro-1-cyclopropyl-1,3- benzodiazol-5- yl)ethynyl]pyrrolo[2,3- d]pyrimidin-7-yl]-2- (methoxymethyl)pyrrolidin-1- yl]prop-2-en-1-one |
| 128 | 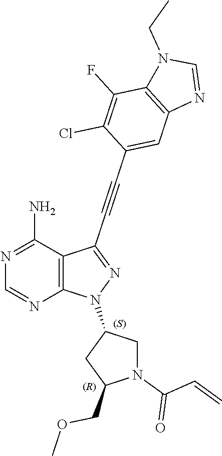 | 1-[(2R,4S)-4-[4-amino-3-[2-(6- chloro-1-ethyl-7-fluoro-1,3- benzodiazol-5- yl)ethynyl]pyrazolo[3,4- d]pyrimidin-1-yl]-2- (methoxymethyl)pyrrolidin-1- yl]prop-2-en-1-one |
| 129 | 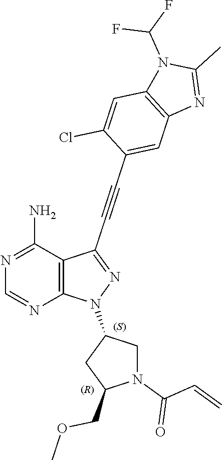 | 1-[(2R,4S)-4-(4-amino-3-{2-[6- chloro-1-(difluoromethyl)-2- methyl-1,3-benzodiazol-5- yl]ethynyl}pyrazolo[3,4- d]pyrimidin-1-yl)-2- (methoxymethyl)pyrrolidin-1- yl]prop-2-en-1-one |
| 130 | 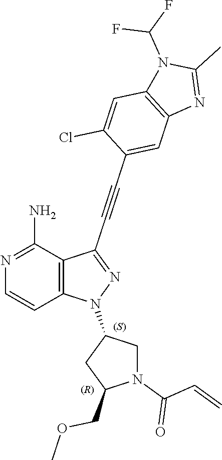 | 1-[(2R,4S)-4-(4-amino-3-{2-[6- chloro-1-(difluoromethyl)-2- methyl-1,3-benzodiazol-5- yl]ethynyl}pyrazolo[4,3- c]pyridin-1-yl)-2- (methoxymethyl)pyrrolidin-1- yl]prop-2-en-1-one |
| 131 | 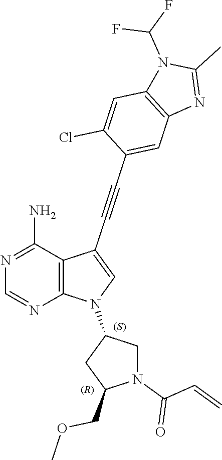 | 1-[(2R,4S)-4-(4-amino-5-{2-[6- chloro-1-(difluoromethyl)-2- methyl-1,3-benzodiazol-5- yl]ethynyl}pyrrolo[2,3- d]pyrimidin-7-yl)-2- (methoxymethyl)pyrrolidin-1- yl]prop-2-en-1-one |
| 132 | 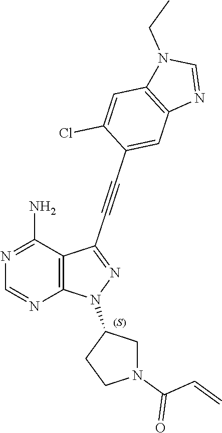 | 1-[(3S)-3-{4-amino-3-[2-(6- chloro-1-ethyl-1,3-benzodiazol- 5-yl)ethynyl]pyrazolo[3,4- d]pyrimidin-1-yl}pyrrolidin-1- yl]prop-2-en-1-one |
| 133 | 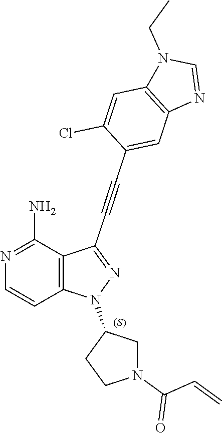 | (S)-1-(3-(4-amino-3-((6-chloro- 1-ethyl-1H-benzo[d]imidazol-5- yl)ethynyl)-1H-pyrazolo[4,3- c]pyridin-1-yl)pyrrolidin-1- yl)prop-2-en-1-one |
| 134 | 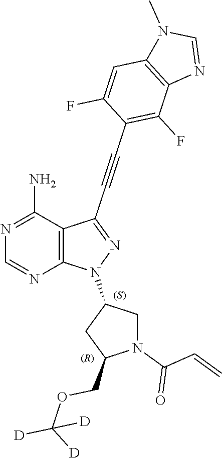 | 1-[(2R,4S)-4-[4-amino-3-[2-(4,6- difluoro-1-methyl-1,3- benzodiazol-5- yl)ethynyl]pyrazolo[3,4- d]pyrimidin-1-yl]-2- [(2H³)methoxymethyl]pyrrolidin- 1-yl]prop-2-en-1-one |
| 135 | 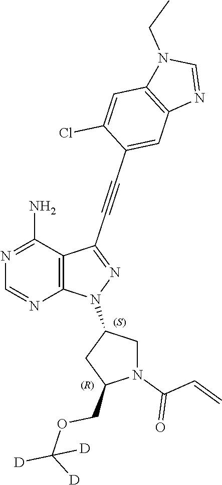 | 1-[(2R,4S)-4-[4-amino-3-[2-(6- chloro-1-ethyl-1,3-benzodiazol- 5-yl)ethynyl]pyrazolo[3,4- d]pyrimidin-1-yl]-2- [(2H³)methoxymethyl]pyrrolidin- 1-yl]prop-2-en-1-one |
| 136 | 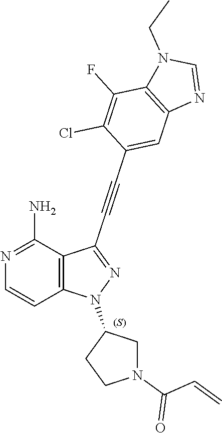 | (S)-1-(3-(4-amino-3-((6-chloro- 1-ethyl-7-fluoro-1H- benzo[d]imidazol-5-yl)ethynyl)- 1H-pyrazolo[4,3-c]pyridin-1- yl)pyrrolidin-1-yl)prop-2-en-1- one |
| 137 | 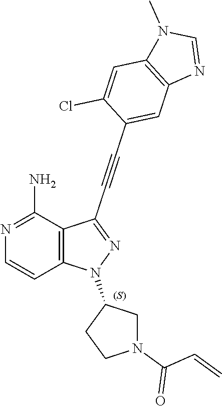 | (S)-1-(3-(4-amino-3-((6-chloro- 1-methyl-1H-benzo[d]imidazol- 5-yl)ethynyl)-1H-pyrazolo[4,3- c]pyridin-1-yl)pyrrolidin-1- yl)prop-2-en-1-one |
| 138 | 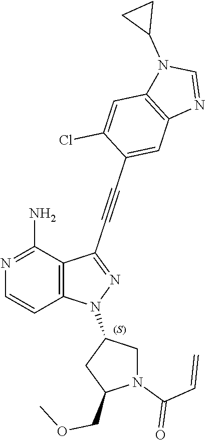 | 1-[(2R,4S)-4-{4-Amino-3-[2-(6- chloro-1-cyclopropyl-1,3- benzodiazol-5- yl)ethynyl]pyrazolo[4,3- c]pyridin-1-yl}-2- (methoxymethyl)pyrrolidin-1- yl]prop-2-en-1-one |
| 139 | 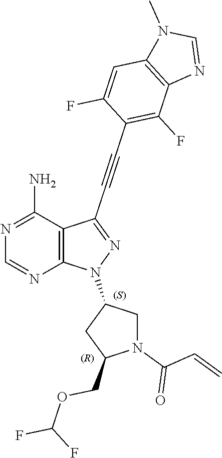 | 1-[(2R,4S)-4-{4-Amino-3-[2- (4,6-difluoro-1-methyl-1,3- benzodiazol-5- yl)ethynyl]pyrazolo[3,4- d]pyrimidin-1-yl}-2- [(difluoromethoxy)methyl] pyrrolidin-1-yl]prop-2-en-1-one |
| 140 | 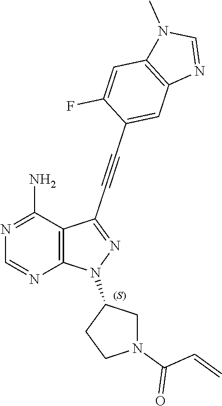 | 1-[(3S)-3-{4-Amino-3-[2-(6- fluoro-1-methyl-1,3- benzodiazol-5- yl)ethynyl]pyrazolo[3,4- d]pyrimidin-1-yl}pyrrolidin-1- yl]prop-2-en-1-one |
| 141 | 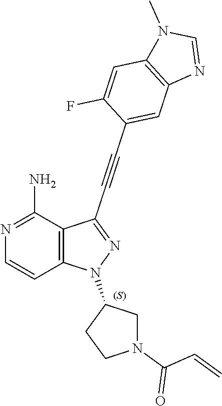 | 1-[(3S)-3-{4-Amino-3-[2-(6- fluoro-1-methyl-1,3- benzodiazol-5- yl)ethynyl]pyrazolo[4,3- c]pyridin-1-yl}pyrrolidin-1- yl ]prop-2-en-1-one |
| 142 | 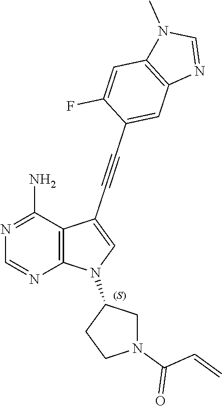 | (S)-1-(3-(4-Amino-5-((6-fluoro- 1-methyl-1H-benzo[d]imidazol- 5-yl)ethynyl)-7H-pyrrolo[2,3- d]pyrimidin-7-yl)pyrrolidin-1- yl)prop-2-en-1-one |
| 143 | 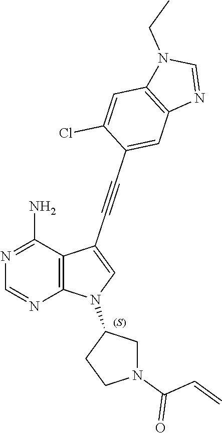 | (S)-1-(3-(4-Amino-5-((6-chloro- 1-ethyl-1H-benzo[d]imidazol-5- yl)ethynyl)-7H-pyrrolo[2,3- d]pyrimidin-7-yl)pyrrolidin-1- yl)prop-2-en-1-one |
| 144 | 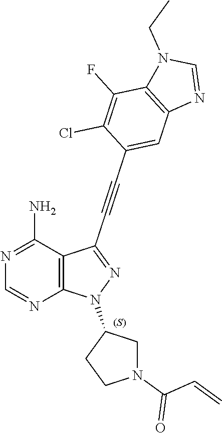 | 1-[(3S)-3-{4-Amino-3-[2-(6- chloro-1-ethyl-7-fluoro-1,3- benzodiazol-5- yl)ethynyl]pyrazolo[3,4- d]pyrimidin-1-yl}pyrrolidin-1- yl]prop-2-en-1-one |
| 145 | 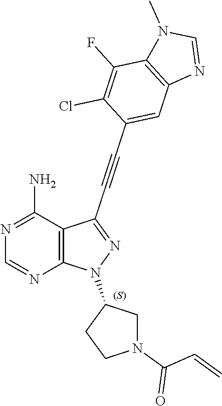 | (S)-1-(3-(4-Amino-3-((6-chloro- 7-fluoro-1-methyl-1H- benzo[d]imidazol-5-yl)ethynyl)- 1H-pyrazolo[3,4-d]pyrimidin-1- yl)pyrrolidin-1-yl)prop-2-en-1- one |
| 146 | 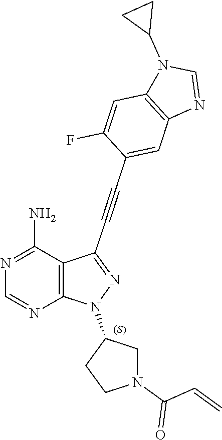 | (S)-1-(3-(4-Amino-3-((1- cyclopropyl-6-fluoro-1H- benzo[d]imidazol-5-yl)ethynyl)- 1H-pyrazolo[3,4-d]pyrimidin-1- yl)pyrrolidin-1-yl)prop-2-en-1- one |
| 147 | 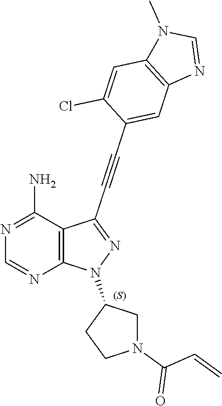 | 1-[(3S)-3-{4-Amino-3-[2-(6- chloro-1-methyl-1,3- benzodiazol-5- yl)ethynyl]pyrazolo[3,4- d]pyrimidin-1-yl}pyrrolidin-1- yl]prop-2-en-1-one |
| 148 | 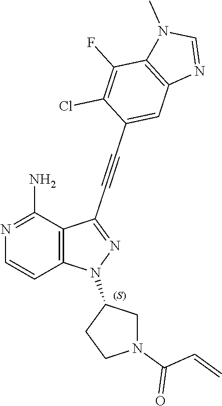 | 1-[(3S)-3-{4-Amino-3-[2-(6- chloro-1-methyl-1,3- benzodiazol-5- yl)ethynyl]pyrazolo[4,3- c]pyridin-1-yl}pyrrolidin-1- yl]prop-2-en-1-one |
| 149 | 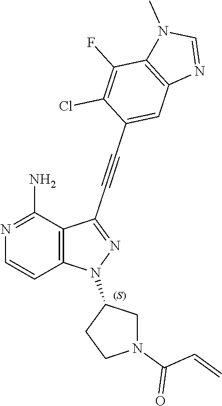 | 1-[(3S)-3-{4-Amino-3-[2-(1- cyclopropyl-6-fluoro-1,3- benzodiazol-5- yl)ethynyl]pyrazolo[4,3- c]pyridin-1-yl}pyrrolidin-1- yl]prop-2-en-1-one |
| 150 | 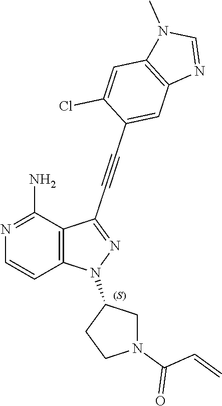 | 1-[(3S)-3-{4-Amino-3-[2-(1- ethyl-6-fluoro-1,3-benzodiazol- 5-yl)ethynyl]pyrazolo[4,3- c]pyridin-1-yl}pyrrolidin-1- yl]prop-2-en-1-one |
| 151 | 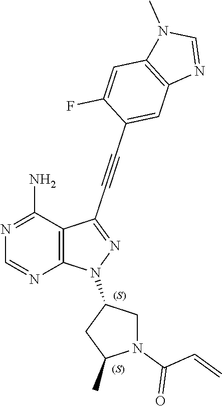 | 1-((2S,4S)-4-(4-amino-3-((6- fluoro-1-methyl-1H- benzo[d]imidazol-5-yl)ethynyl)- 1H-pyrazolo[3,4-d]pyrimidin-1- yl)-2-methylpyrrolidin-1- yl)prop-2-en-1-one |
| 152 | 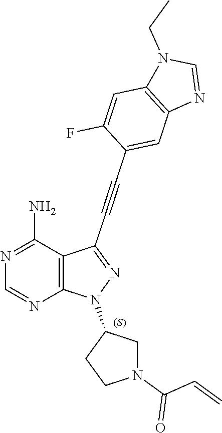 | 1-[(3S)-3-{4-Amino-3-[2-(1- ethyl-6-fluoro-1,3-benzodiazol- 5-yl)ethynyl]pyrazolo[3,4- d]pyrimidin-1-yl}pyrrolidin-1- yl]prop-2-en-1-one |
| 153 | 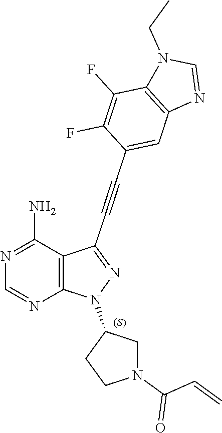 | 1-[(3S)-3-{4-Amino-3-[2-(1- ethyl-6,7-difluoro-1,3- benzodiazol-5- yl)ethynyl]pyrazolo[3,4- d]pyrimidin-1-yl}pyrrolidin-1- yl]prop-2-en-1-one |
| 154 | 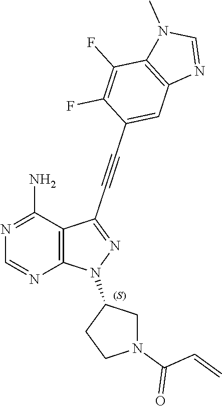 | 1-[(3S)-3-{4-Amino-3-[2-(6,7- difluoro-1-methyl-1,3- benzodiazol-5- yl)ethynyl]pyrazolo[3,4- d]pyrimidin-1-yl}pyrrolidin-1- yl]prop-2-en-1-one |
| 155 | 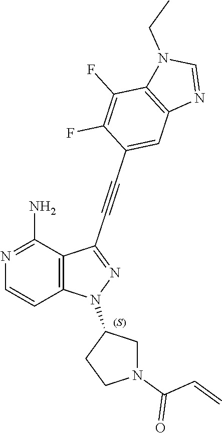 | 1-[(3S)-3-{4-Amino-3-[2-(1- ethyl-6,7-difluoro-1,3- benzodiazol-5- yl)ethynyl]pyrazolo[4,3- c]pyridin-1-yl}pyrrolidin-1- yl]prop-2-en-1-one |
| 156 | 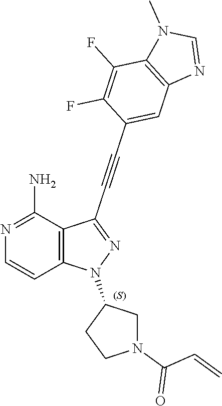 | 1-[(3S)-3-{4-Amino-3-[2-(6,7- difluoro-1-methyl-1,3- benzodiazol-5- yl)ethynyl]pyrazolo[4,3- c]pyridin-1-yl}pyrrolidin-1- yl]prop-2-en-1-one |
| 157 | 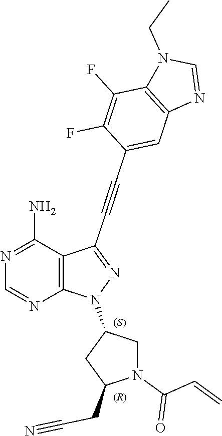 | 2-[(2R,4S)-4-{4-amino-3-[2-(1- ethyl-6,7-difluoro-1,3- benzodiazol-5- yl)ethynyl]pyrazolo[3,4- d]pyrimidin-1-yl}-1-(prop-2- enoyl)pyrrolidin-2-yl]acetonitrile |
| 158 | 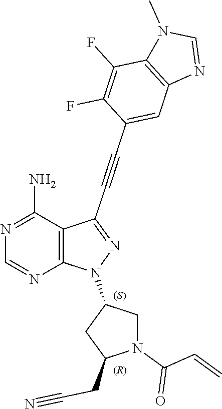 | 2-[(2R,4S)-4-{4-Amino-3-[2- (6,7-difluoro-1-methyl-1,3- benzodiazol-5- yl)ethynyl]pyrazolo[3,4- d]pyrimidin-1-yl}-1-(prop-2- enoyl)pyrrolidin-2-yl]acetonitrile |
| 159 | 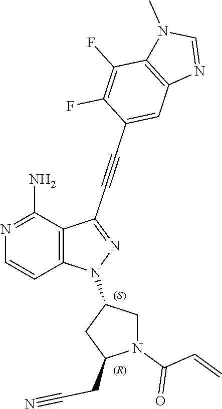 | 2-((2R,4S)-1-Acryloyl-4-(4- amino-3-((6,7-difluoro-1-methyl- 1H-benzo[d]imidazol-5- yl)ethynyl)-1H-pyrazolo[4,3- c]pyridin-1-yl)pyrrolidin-2- yl)acetonitrile |
| 160 | 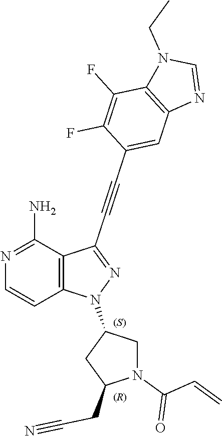 | 2-[(2R,4S)-4-{4-Amino-3-[2-(1- ethyl-6,7-difluoro-1,3- benzodiazol-5- yl)ethynyl]pyrazolo[4,3- c]pyridin-1-yl}-1-(prop-2- enoyl)pyrrolidin-2-yl]acetonitrile |
| 161 | 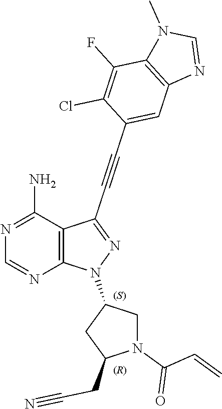 | 2-[(2R,4S)-4-{4-Amino-3-[2-(6- chloro-7-fluoro-1-methyl-1,3- benzodiazol-5- yl)ethynyl]pyrazolo[3,4- d]pyrimidin-1-yl}-1-(prop-2- enoyl)pyrrolidin-2-yl]acetonitrile |
| 162 | 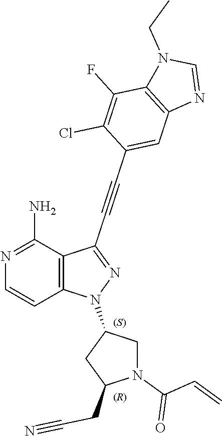 | 2-[(2R,4S)-4-{4-Amino-3-[2-(6- chloro-1-ethyl-7-fluoro-1,3- benzodiazol-5- yl)ethynyl]pyrazolo[4,3- c]pyridin-1-yl}-1-(prop-2- enoyl)pyrrolidin-2-yl]acetonitrile |
| 163 | 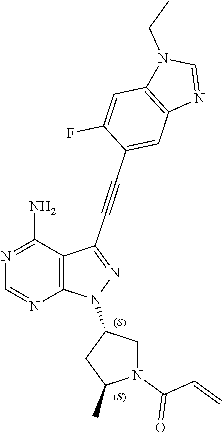 | 1-((2S,4S)-4-(4-amino-3-((1- ethyl-6-fluoro-1H- benzo[d]imidazol-5-yl)ethynyl)- 1H-pyrazolo[3,4-d]pyrimidin-1- yl)-2-methylpyrrolidin-1- yl)prop-2-en-1-one |
| 164 | 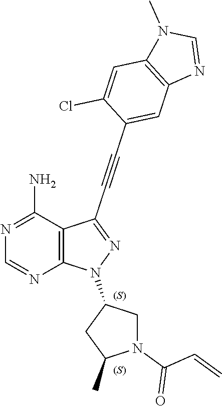 | 1-((2S,4S)-4-(4-amino-3-((6- chloro-1-methyl-1H- benzo[d]imidazol-5-yl)ethynyl)- 1H-pyrazolo[3,4-d]pyrimidin-1- yl)-2-methylpyrrolidin-1- yl)prop-2-en-1-one |
| 165 | 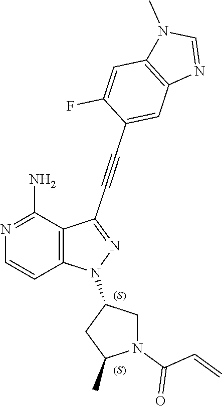 | 1-((2S,4S)-4-(4-amino-3-((6- fluoro-1-methyl-1H- benzo[d]imidazol-5-yl)ethynyl)- 1H-pyrazolo[4,3-c]pyridin-1-yl)- 2-methylpyrrolidin-1-yl)prop-2- en-1-one |
| 166 | 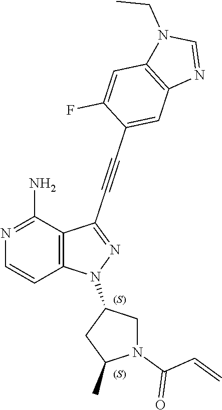 | 1-((2S,4S)-4-(4-amino-3-((1- ethyl-6-fluoro-1H- benzo[d]imidazol-5-yl)ethynyl)- 1H-pyrazolo[4,3-c]pyridin-1-yl)- 2-methylpyrrolidin-1-yl)prop-2- en-1-one |
| 167 | 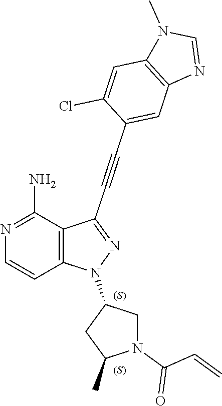 | 1-((2S,4S)-4-(4-amino-3-((6- chloro-1-methyl-1H- benzo[d]imidazol-5-yl)ethynyl)- 1H-pyrazolo[4,3-c]pyridin-1-yl)- 2-methylpyrrolidin-1-yl)prop-2- en-1-one |
| 168 | 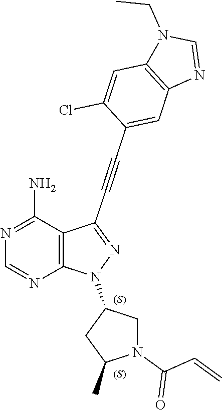 | 1-((2S,4S)-4-(4-amino-3-((6- chloro-1-ethyl-1H- benzo[d]imidazol-5-yl)ethynyl)- 1H-pyrazolo[3,4-d]pyrimidin-1- yl)-2-methylpyrrolidin-1- yl)prop-2-en-1-one |
| 169 | 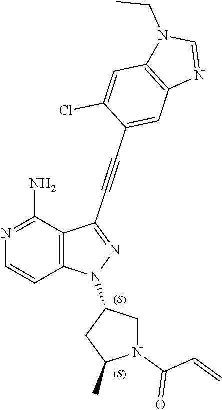 | 1-((2S,4S)-4-(4-amino-3-((6- chloro-1-ethyl-1H- benzo[d]imidazol-5-yl)ethynyl)- 1H-pyrazolo[4,3-c]pyridin-1-yl)- 2-methylpyrrolidin-1-yl)prop-2- en-1-one |
| 170 | 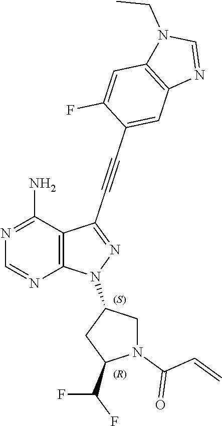 | 1-((2R,4S)-4-(4-amino-3-((1- ethyl-6-fluoro-1H- benzo[d]imidazol-5-yl)ethynyl)- 1H-pyrazolo[3,4-d]pyrimidin-1- yl)-2-(difluoromethyl)pyrrolidin- 1-yl)prop-2-en-1-one |
| 171 | 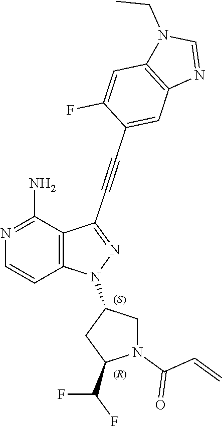 | 1-((2R,4S)-4-(4-amino-3-((1- ethyl-6-fluoro-1H- benzo[d]imidazol-5-yl)ethynyl)- 1H-pyrazolo[4,3-c]pyridin-1-yl)- 2-(difluoromethyl)pyrrolidin-1- yl)prop-2-en-1-one |
| 172 | 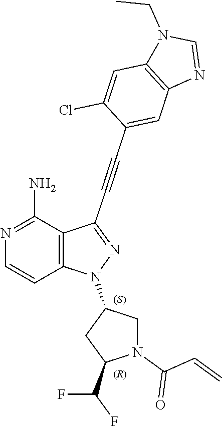 | 1-((2R,4S)-4-(4-amino-3-((6- chloro-1-methyl-1H- benzo[d]imidazol-5-yl)ethynyl)- 1H-pyrazolo[4,3-c]pyridin-1-yl)- 2-(difluoromethyl)pyrrolidin-1- yl)prop-2-en-1-one |
| 173 | 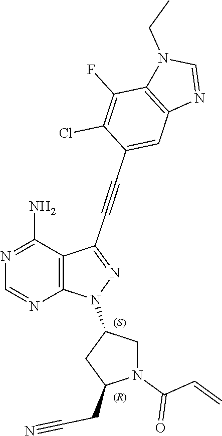 | 2-[(2R,4S)-4-{4-Amino-3-[2-(6- chloro-1-ethyl-7-fluoro-1,3- benzodiazol-5- yl)ethynyl]pyrazolo[3,4- d]pyrimidin-1-yl}-1-(prop-2- enoyl)pyrrolidin-2-yl]acetonitrile |
| 174 | 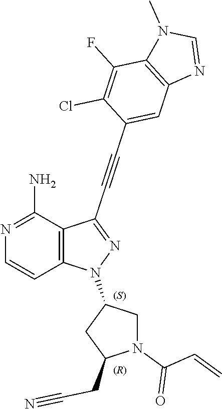 | 2-[(2R,4S)-4-{4-Amino-3-[2-(6- chloro-7-fluoro-1-methyl-1,3- benzodiazol-5- yl)ethynyl]pyrazolo[4,3- c]pyridin-1-yl}-1-(prop-2- enoyl)pyrrolidin-2-yl]acetonitrile |
| 175 | 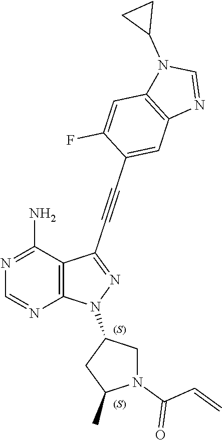 | 1-((2S,4S)-4-(4-amino-3-((1- cyclopropyl-6-fluoro-1H- benzo[d]imidazol-5-yl)ethynyl)- 1H-pyrazolo[3,4-d]pyrimidin-1- yl)-2-methylpyrrolidin-1- yl)prop-2-en-1-one |
| 176 | 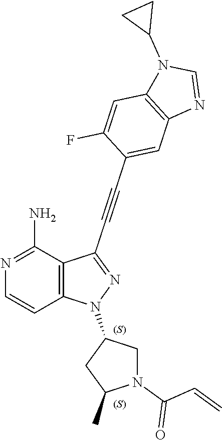 | 1-((2S,4S)-4-(4-amino-3-((1- cyclopropyl-6-fluoro-1H- benzo[d]imidazol-5-yl)ethynyl)- 1H-pyrazolo[4,3-c]pyridin-1-yl)- 2-methylpyrrolidin-1-yl)prop-2- en-1-one |
| 177 | 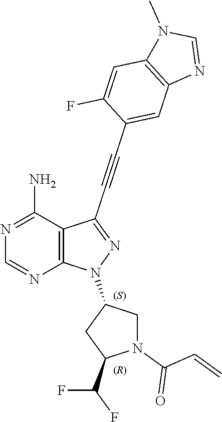 | 1-((2R,4S)-4-(4-amino-3-((6- fluoro-1-methyl-1H- benzo[d]imidazol-5-yl)ethynyl)- 1H-pyrazolo[3,4-d]pyrimidin-1- yl)-2-(difluoromethyl)pyrrolidin- 1-yl)prop-2-en-1-one |
| 178 | 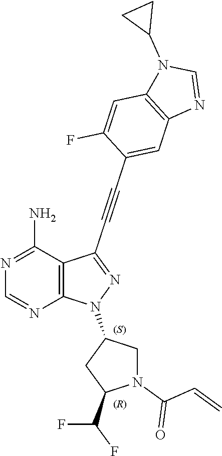 | 1-((2R,4S)-4-(4-amino-3-((1- cyclopropyl-6-fluoro-1H- benzo[d]imidazol-5-yl)ethynyl)- 1H-pyrazolo[3,4-d]pyrimidin-1- yl)-2-(difluoromethyl)pyrrolidin- 1-yl)prop-2-en-1-one |
| 179 | 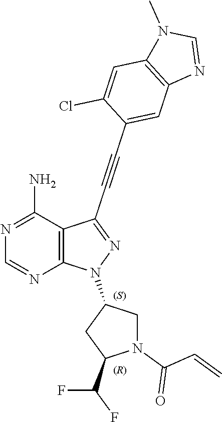 | 1-((2R,4S)-4-(4-amino-3-((6- chloro-1-methyl-1H- benzo[d]imidazol-5-yl)ethynyl)- 1H-pyrazolo[3,4-d]pyrimidin-1- yl)-2-(difluoromethyl)pyrrolidin- 1-yl)prop-2-en-1-one |
| 180 | 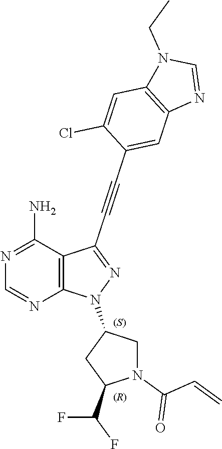 | 1-((2R,4S)-4-(4-amino-3-((6- chloro-1-methyl-1H- benzo[d]imidazol-5-yl)ethynyl)- 1H-pyrazolo[3,4-d]pyrimidin-1- yl)-2-(difluoromethyl)pyrrolidin- 1-yl)prop-2-en-1-one |
| 181 | 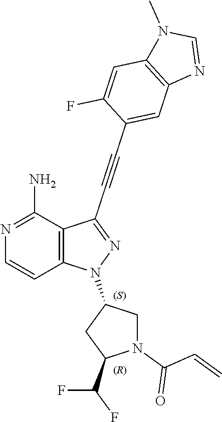 | 1-((2R,4S)-4-(4-amino-3-((6- fluoro-1-methyl-1H- benzo[d]imidazol-5-yl)ethynyl)- 1H-pyrazolo[4,3-c]pyridin-1-yl)- 2-(difluoromethyl)pyrrolidin-1- yl)prop-2-en-1-one |
| 182 | 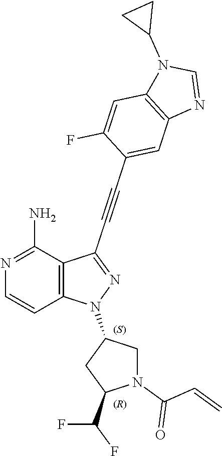 | 1-((2R,4S)-4-(4-amino-3-((1- cyclopropyl-6-fluoro-1H- benzo[d]imidazol-5-yl)ethynyl)- 1H-pyrazolo[4,3-c]pyridin-1-yl)- 2-(difluoromethyl)pyrrolidin-1- yl)prop-2-en-1-one |
| 183 | 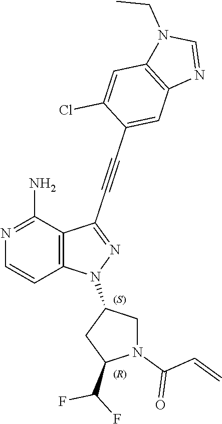 | 1-((2R,4S)-4-(4-amino-3-((6- chloro-1-ethyl-1H- benzo[d]imidazol-5-yl)ethynyl)- 1H-pyrazolo[4,3-c]pyridin-1-yl)- 2-(difluoromethyl)pyrrolidin-1- yl)prop-2-en-1-one |
| 184 | 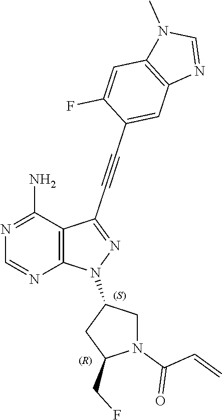 | 1-[(2R,4S)-4-{4-Amino-3-[2-(6- fluoro-1-methyl-1,3- benzodiazol-5- yl)ethynyl]pyrazolo[3,4- d]pyrimidin-1-yl}-2- (fluoromethyl)pyrrolidin-1- yl]prop-2-en-1-one |
| 185 | 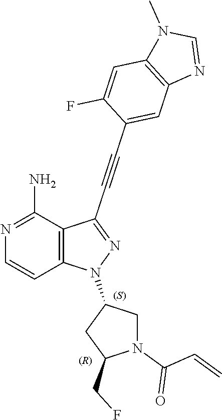 | 1-[(2R,4S)-4-{4-Amino-3-[2-(6- fluoro-1-methyl-1,3- benzodiazol-5- yl)ethynyl]pyrazolo[4,3- c]pyridin-1-yl}-2- (fluoromethyl)pyrrolidin-1- yl]prop-2-en-1-one |
| 186 | 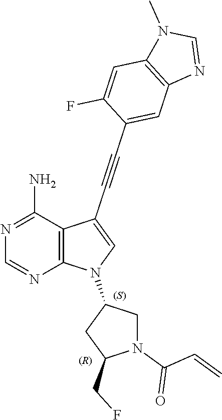 | 1-((2R,4S)-4-(4-Amino-5-((6- fluoro-1-methyl-1H- benzo[d]imidazol-5-yl)ethynyl)- 7H-pyrrolo[2,3-d]pyrimidin-7- yl)-2-(fluoromethyl)pyrrolidin-1- yl)prop-2-en-1-one |
| 187 | 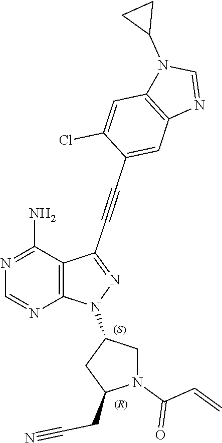 | 2-[(2R,4S)-4-{4-Amino-3-[2-(6- chloro-1-cyclopropyl-1,3- benzodiazol-5- yl)ethynyl]pyrazolo[3,4- d]pyrimidin-1-yl}-1-(prop-2- enoyl)pyrrolidin-2-yl]acetonitrile |
| 188 | 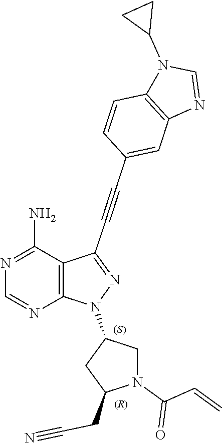 | 2-[(2R,4S)-4-{4-Amino-3-[2-(1- cyclopropyl-1,3-benzodiazol-5- yl)ethynyl]pyrazolo[3,4- d]pyrimidin-1-yl}-1-(prop-2- enoyl)pyrrolidin-2-yl]acetonitrile |
| 189 | 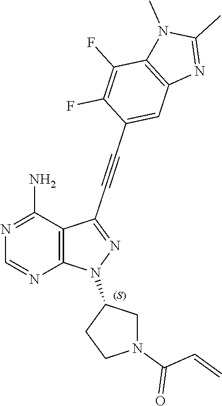 | 1-[(3S)-3-{4-Amino-3-[2-(6,7- difluoro-1,2-dimethyl-1,3- benzodiazol-5- yl)ethynyl]pyrazolo[3,4- d]pyrimidin-1-yl}pyrrolidin-1- yl]prop-2-en-1-one |
| 190 | 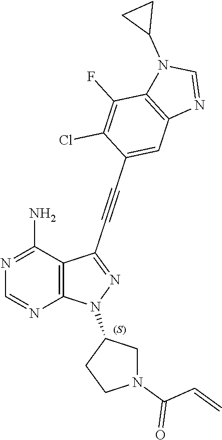 | 1-[(3S)-3-{4-amino-3-[2-(6- chloro-1-cyclopropyl-7-fluoro- 1,3-benzodiazol-5- yl)ethynyl]pyrazolo[3,4- d]pyrimidin-1-yl}pyrrolidin-1- yl]prop-2-en-1-one |
| 191 | 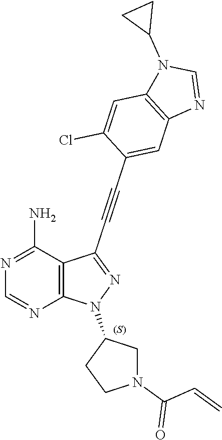 | 1-[(3S)-3-{4-Amino-3-[2-(6- chloro-1-cyclopropyl-1,3- benzodiazol-5- yl)ethynyl]pyrazolo[3,4- d]pyrimidin-1-yl}pyrrolidin-1- yl]prop-2-en-1-one |
| 192 | 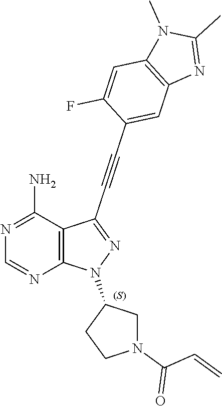 | 1-[(3S)-3-{4-Amino-3-[2-(6- fluoro-1,2-dimethyl-1,3- benzodiazol-5- yl)ethynyl]pyrazolo[3,4- d]pyrimidin-1-yl}pyrrolidin-1- yl]prop-2-en-1-one |
| 193 | 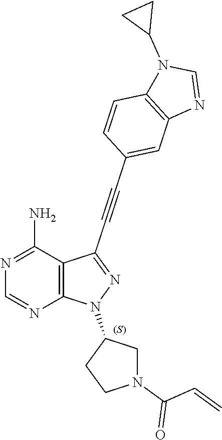 | 1-[(3S)-3-{4-Amino-3-[2-(1- cyclopropyl-1,3-benzodiazol-5- yl)ethynyl]pyrazolo[3,4- d]pyrimidin-1-yl}pyrrolidin-1- yl]prop-2-en-1-one |
| 194 | 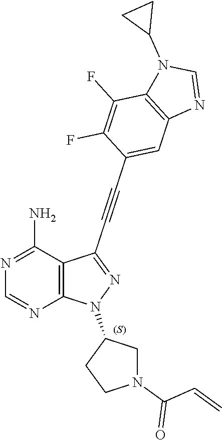 | 1-[(3S)-3-{4-Amino-3-[2-(1- cyclopropyl-6,7-difluoro-1,3- benzodiazol-5- yl)ethynyl]pyrazolo[3,4- d]pyrimidin-1-yl}pyrrolidin-1- yl]prop-2-en-1-one |
| 195 | 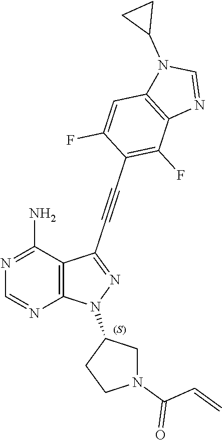 | 1-[(3S)-3-{4-Amino-3-[2-(1- cyclopropyl-4,6-difluoro-1,3- benzodiazol-5- yl)ethynyl]pyrazolo[3,4- d]pyrimidin-1-yl}pyrrolidin-1- yl]prop-2-en-1-one |
| 196 | 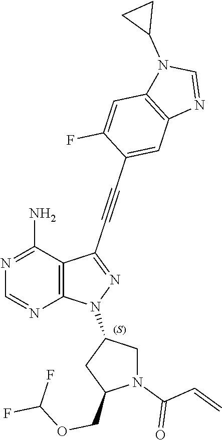 | 1-((2R,4S)-4-(4-Amino-3-((1- cyclopropyl-6-fluoro-1H- benzo[d]imidazol-5-yl)ethynyl)- 1H-pyrazolo[3,4-d]pyrimidin-1- yl)-2- ((difluoromethoxy)methyl) pyrrolidin-1-yl)prop-2-en-1-one |
| 197 | 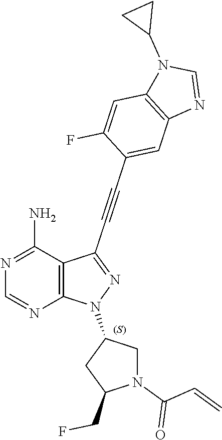 | 1-((2R,4S)-4-(4-Amino-3-((1- cyclopropyl-6-fluoro-1H- benzo[d]imidazol-5-yl)ethynyl)- 1H-pyrazolo[3,4-d]pyrimidin-1- yl)-2-(fluoromethyl)pyrrolidin-1- yl)prop-2-en-1-one |
| 198 | 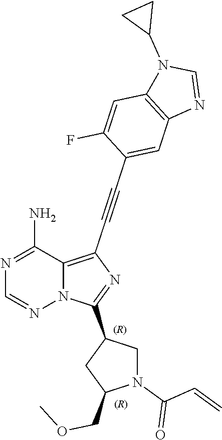 | 1-[(2R,4R)-4-{4-Amino-5-[2-(1- cyclopropyl-6-fluoro-1,3- benzodiazol-5- yl)ethynyl]imidazo[4,3- f][1,2,4]triazin-7-yl}-2- (methoxymethyl)pyrrolidin-1- yl]prop-2-en-1-one |
| 199 | 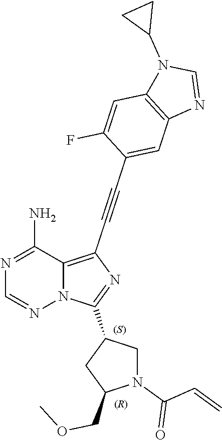 | 1-[(2R,4S)-4-{4-Amino-5-[2-(1- cyclopropyl-6-fluoro-1,3- benzodiazol-5- yl)ethynyl]imidazo[4,3- f][1,2,4]triazin-7-yl}-2- (methoxymethyl)pyrrolidin-1- yl]prop-2-en-1-one |
| 200 | 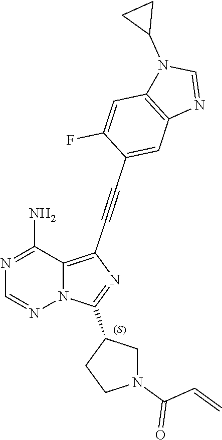 | (S)-1-(3-(4-Amino-5-((1- cyclopropyl-6-fluoro-1H- benzo[d]imidazol-5- yl)ethynyl)imidazo[5,1- f][1,2,4]triazin-7-yl)pyrrolidin-1- yl)prop-2-en-1-one |
| 201 | 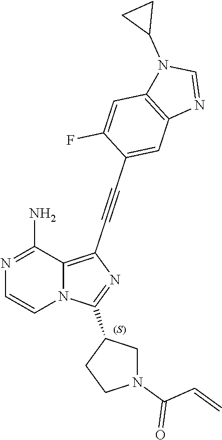 | 1-[(3S)-3-{8-amino-1-[2-(1- cyclopropyl-6-fluoro-1,3- benzodiazol-5- yl)ethynyl]imidazo[1,5- a]pyrazin-3-yl}pyrrolidin-1- yl]prop-2-en-1-one |
| 202 | 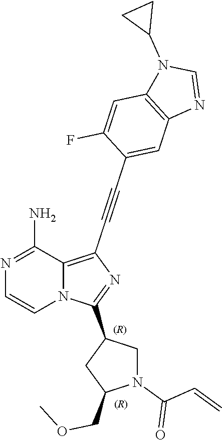 | 1-[(2R,4R)-4-{8-Amino-1-[2-(1- cyclopropyl-6-fluoro-1,3- benzodiazol-5- yl)ethynyl]imidazo[1,5- a]pyrazin-3-yl}-2- (methoxymethyl)pyrrolidin-1- yl]prop-2-en-1-one |
| 203 | 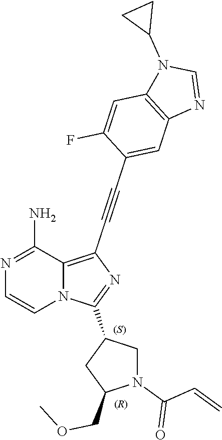 | 1-[(2R,4S)-4-{8-Amino-1-[2-(1- cyclopropyl-6-fluoro-1,3- benzodiazol-5- yl)ethynyl]imidazo[1,5- a]pyrazin-3-yl}-2- (methoxymethyl)pyrrolidin-1- yl]prop-2-en-1-one |

Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Pharmaceutical Compositions

In certain embodiments, the heteroaromatic FGFR kinase inhibitory compound described herein is administered as a pure chemical. In other embodiments, the heteroaromatic FGFR kinase inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one heteroaromatic FGFR kinase inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the heteroaromatic FGFR kinase inhibitory compound as described by Formula (I), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Formula (II), or a pharmaceutically acceptable salt or solvate thereof.

One embodiment provides a method of preparing a pharmaceutical composition comprising mixing a compound of Formula (II), or a pharmaceutically acceptable salt or solvate thereof, and a pharmaceutically acceptable carrier.

In certain embodiments, the heteroaromatic FGFR kinase inhibitory compound as described by Formula (II), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the heteroaromatic FGFR kinase inhibitory compound as described by Formula (I), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

In some embodiments, the heteroaromatic FGFR kinase inhibitory compound as described by Formula (II), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heteroaromatic FGFR kinase inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt or solvate thereof. In some embodiments, described herein is a method of treating cancer in a patient in need thereof comprising administering to the patient a pharmaceutical composition comprising a compound of Formula (I), or a pharmaceutically acceptable salt or solvate thereof, and a pharmaceutically acceptable excipient.

One embodiment provides a compound of Formula (II), or a pharmaceutically acceptable salt or solvate thereof, for use in a method of treatment of the human or animal body.

One embodiment provides a compound of Formula (II), or a pharmaceutically acceptable salt or solvate thereof, for use in a method of treatment of cancer or neoplastic disease.

One embodiment provides a use of a compound of Formula (II), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (II), or a pharmaceutically acceptable salt or solvate thereof. In some embodiments, described herein is a method of treating cancer in a patient in need thereof comprising administering to the patient a pharmaceutical composition comprising a compound of Formula (II), or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the heteroaromatic FGFR kinase inhibitory compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
T temperature
TFA trifluoroacetic acid
THF tetrahydrofuran

### Intermediate 1: 1-[(2R,4S)-4-[4-amino-5-iodopyrrolo[2,3-d]pyrimidin-7-yl]-2-(methoxymethyl)pyrrolidin-1-yl]prop-2-en-1-one

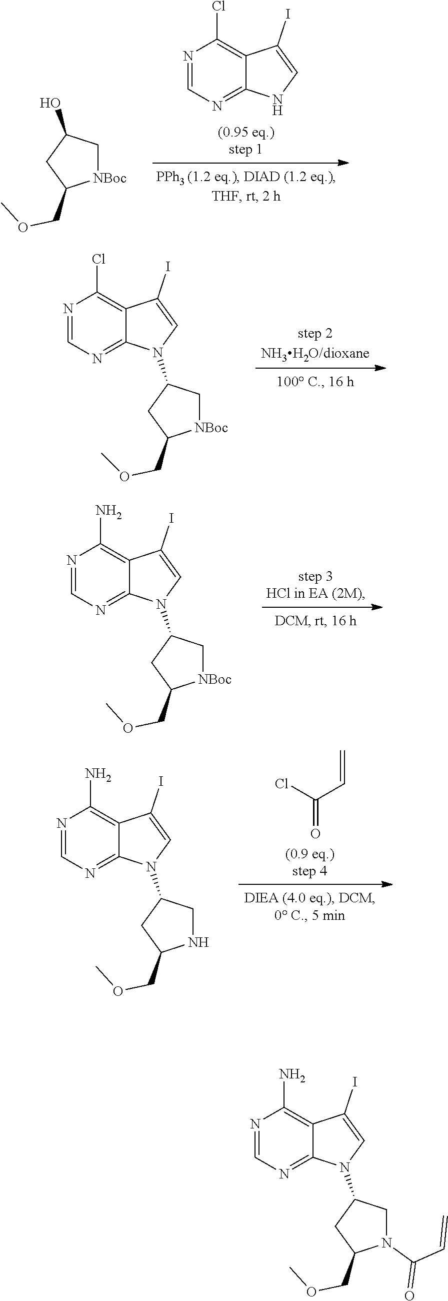

### Step 1: Tert-butyl (2R,4S)-4-[4-chloro-5-iodopyrrolo[2,3-d]pyrimidin-7-yl]-2-(methoxymethyl)pyrrolidine-1-carboxylate

To a stirred solution of tert-butyl (2R,4R)-4-hydroxy-2-(methoxymethyl)pyrrolidine-1-carboxylate (40.00 g, 172.94 mmol), 4-chloro-5-iodo-7H-pyrrolo[2,3-d]pyrimidine (45.92 g, 164.29 mmol) and PPh3 (54.43 g, 207.53 mmol) in THF (500.00 mL) was added DIAD (41.14 mL, 203.46 mmol) dropwise at 0° C. under argon atmosphere. The reaction mixture was degassed with argon for three times and stirred for 2 h at room temperature. The resulting mixture was concentrated under vacuum for 60 min at 25° C. The residue was diluted with EA (2000 mL). The resulting mixture was washed with water (3×1000 mL) and brine (1000 mL). The organic layer was dried over anhydrous Na2SO4 (500 g) and filtered. The filtrate was concentrated under reduced pressure. The residue was purified by silica gel column chromatography, eluted with EA in PE (10-45%). The fractions that contained desired product were combined and concentrated to afford tert-butyl (2R,4S)-4-[4-chloro-5-iodopyrrolo[2,3-d]pyrimidin-7-yl]-2-(methoxymethyl)pyrrolidine-1-carboxylate (80 g, 94%) as a light yellow solid. MS ESI calculated for C17H22ClIN4O3 [M+H]+, 493.04, found 493.10.

### Step 2: Tert-butyl (2R,4S)-4-[4-amino-5-iodopyrrolo[2,3-d]pyrimidin-7-yl]-2-(methoxymethyl)pyrrolidine-1-carboxylate

To a steel sealed tube was added tert-butyl (2R,4S)-4-[4-chloro-5-iodopyrrolo[2,3-d]pyrimidin-7-yl]-2-(methoxymethyl)pyrrolidine-1-carboxylate (40.00 g, 81.18 mmol), dioxane (100 mL) and NH3·H2O (300 mL). The reaction mixture was stirred for 16 h at 100° C. The resulting mixture was concentrated under reduced pressure for 60 min at 25° C. to dryness. The residue was purified by silica gel column chromatography, eluted with MeOH in DCM (0-4.5%). The fractions that contained desired product were combined and concentrated to afford tert-butyl (2R,4S)-4-[4-amino-5-iodopyrrolo[2,3-d]pyrimidin-7-yl]-2-(methoxymethyl)pyrrolidine-1-carboxylate (32.3 g, 84%) as a light yellow solid. MS ESI calculated for C17H24IN5N3 [M+H]+, 474.09; found 474.25.

### Step 3: 5-Iodo-7-[(3S,5R)-5-(methoxymethyl)pyrrolidin-3-yl]pyrrolo[2,3-d]pyrimidin-4-amine

A mixture of tert-butyl (2R,4S)-4-[4-amino-5-iodopyrrolo[2,3-d]pyrimidin-7-yl]-2-(methoxymethyl)pyrrolidine-1-carboxylate (1.79 g, 3.78 mmol) in 2 M hydrogen chloride solution in EA (20.00 mL, 40.00 mmol) and DCM (20.00 mL) was stirred 16 h at room temperature under argon atmosphere. The resulting mixture was concentrated under vacuum. The residue was purified by reverse phase column chromatography with the following conditions: column, AQ silica gel; mobile phase, MeCN in water (10 mmol/L of NH4HCO3), 10% to 35% gradient in 25 min. The fractions that contained desired product were combined and concentrated to afford 5-iodo-7-[(3S,5R)-5-(methoxymethyl)pyrrolidin-3-yl]pyrrolo[2,3-d]pyrimidin-4-amine (0.90 g, 64%) as a light yellow solid. MS ESI calculated for C12H16IN5O [M+H]+, 374.04, found 374.05.

### Step 4: 1-[(2R,4S)-4-[4-amino-5-iodopyrrolo[2,3-d]pyrimidin-7-yl]-2-(methoxymethyl)pyrrolidin-1-yl]prop-2-en-1-one

To a stirred solution of 5-iodo-7-[(3S,5R)-5-(methoxymethyl)pyrrolidin-3-yl]pyrrolo[2,3-d]pyrimidin-4-amine (0.80 g, 2.14 mmol) and DIEA (1.52 mL, 11.76 mmol) in DCM (12.00 mL) was added acryloyl chloride (0.62 mL, 1.93 mmol) dropwise at 0° C. under argon atmosphere. The reaction mixture was stirred for 10 min at 0° C. under argon atmosphere. The reaction was quenched with water (30 mL) at 0° C. The resulting mixture was extracted with DCM (3×20 mL). The combined organic layers were dried over anhydrous Na2SO4 and filtered. The filtrate was concentrated under reduced pressure. The residue was purified by silica gel column chromatography, eluted with EA in PE (10-50%). The fractions that contained desired product were combined and concentrated to afford 1-[(2R,4S)-4-[4-amino-5-iodopyrrolo[2,3-d]pyrimidin-7-yl]-2-(methoxymethyl)pyrrolidin-1-yl]prop-2-en-1-one (0.78 g, 85%) as an off-white solid. MS ESI calculated for C15H18IN5O2 [M+H]+, 428.05; found 428.10.

### Intermediate 2: 1-((2R,4S)-4-(4-amino-3-iodo-1H-pyrazolo[3,4-d]pyrimidin-1-yl)-2-(methoxymethyl)pyrrolidin-1-yl)prop-2-en-1-one

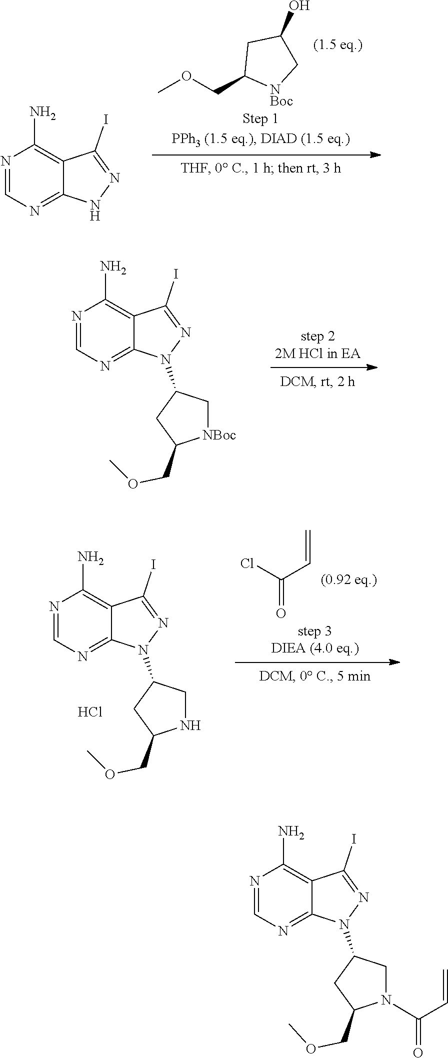

### Step 1: Tert-butyl (2R,4S)-4-(4-amino-3-iodo-1H-pyrazolo[3,4-d]pyrimidin-1-yl)-2-(methoxymethyl)pyrrolidine-1-carboxylate

To a stirred solution of 3-iodo-1H-pyrazolo[3,4-d]pyrimidin-4-amine (5.00 g, 19.16 mmol) and tert-butyl (2R,4R)-4-hydroxy-2-(methoxymethyl)pyrrolidine-1-carboxylate (6.65 g, 28.75 mmol) in THF (500 mL) were added PPh3 (7.54 g, 28.75 mmol) and DIAD (5.81 g, 28.75 mmol) at 0° C. under nitrogen atmosphere. The reaction mixture was stirred for 1 h at 0° C. and for 3 h at room temperature. The resulting mixture was concentrated under reduced pressure. The residue was diluted with EA (100 mL). The resulting mixture was washed with water (3×70 mL) and brine (100 mL). The organic layer was dried over Na2SO4 and filtered. The filtrate was concentrated under reduced pressure. The residue was purified by silica gel column chromatography, eluted with MeOH in DCM (0-6%). The fractions that contained desired product were combined and concentrated to afford tert-butyl (2R,4S)-4-(4-amino-3-iodo-1H-pyrazolo[3,4-d]pyrimidin-1-yl)-2-(methoxymethyl)pyrrolidine-1-carboxylate (6.8 g, 74%) as a light yellow solid. MS ESI calculated for C16H23IN6O3 [M+H]+, 475.09, found 475.15.

### Step 2: 3-iodo-1-((3S,5R)-5-(methoxymethyl)pyrrolidin-3-yl)-1H-pyrazolo[3,4-d]pyrimidin-4-amine hydrochloride

To a stirred solution of tert-butyl (2R,4S)-4-[4-amino-3-iodopyrazolo[3,4-d]pyrimidin-1-yl]-2-(methoxymethyl)pyrrolidine-1-carboxylate (1.50 g, 3.16 mmol) in DCM (17.00 mL) was added 2 M hydrogen chloride solution in EA (35 mL) dropwise at 0° C. under nitrogen atmosphere. The reaction mixture was stirred for 2 h at room temperature under nitrogen atmosphere. The resulting mixture was concentrated under reduced pressure. After filtration, the filtrate was concentrated under reduced pressure to afford 3-iodo-1-((3S,5R)-5-(methoxymethyl)pyrrolidin-3-yl)-1H-pyrazolo[3,4-d]pyrimidin-4-amine hydrochloride (1.3 g, 100%) as an off-white solid. MS ESI calculated for C11H15IN6O [M+H]+, 375.04, found 375.08.

### Step 3: 1-((2R,4S)-4-(4-amino-3-iodo-1H-pyrazolo[3,4-d]pyrimidin-1-yl)-2-(methoxymethyl)pyrrolidin-1-yl)prop-2-en-1-one

To a stirred solution of 3-iodo-1-((3S,5R)-5-(methoxymethyl)pyrrolidin-3-yl)-1H-pyrazolo[3,4-d]pyrimidin-4-amine (1.25 g, 2.80 mmol) in DCM (28.00 mL) were added acryloyl chloride (0.23 g, 2.57 mmol) and DIEA (1.95 mL, 11.20 mmol,) dropwise at 0° C. The reaction mixture was stirred for 5 min at 0° C. The resulting mixture was diluted with water (30 mL). The resulting mixture was extracted with DCM (3×30 mL). The combined organic layers were washed with brine (3×20 mL), dried over anhydrous Na2SO4 and filtered. The filtrate was concentrated under reduced pressure. The residue was purified by reverse phase column chromatography, eluted with 43% ACN in water (10 mmol/L NH4HCO3). The fractions that contained desired product were combined and concentrated to afford 1-((2R,4S)-4-(4-amino-3-iodo-1H-pyrazolo[3,4-d]pyrimidin-1-yl)-2-(methoxymethyl)pyrrolidin-1-yl)prop-2-en-1-one (1.07 g, 89%) as an off-white solid. MS ESI calculated for C14H17IN6O2 [M+H]+, 429.05, found 429.10.

### Intermediate 3: 1-((2R,4S)-4-(4-amino-3-iodo-1H-pyrazolo[4,3-c]pyridin-1-yl)-2-(methoxymethyl)pyrrolidin-1-yl)prop-2-en-1-one

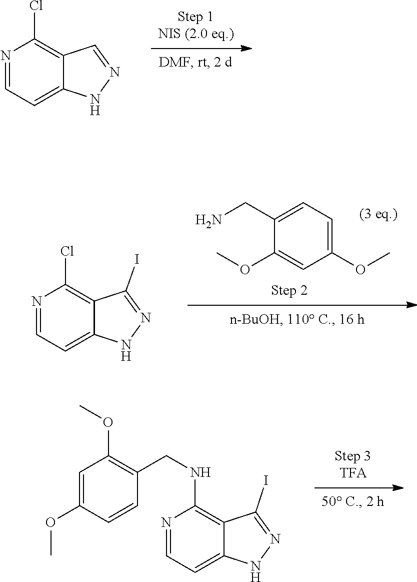 [SMILES: Clc1nccc2[nH]ncc12 ; Clc1nccc2[nH]nc(I)c12 ; COc1ccc(CN)c(OC)c1 ; COc1ccc(CNc2nccc3[nH]nc(I)c23)c(OC)c1]

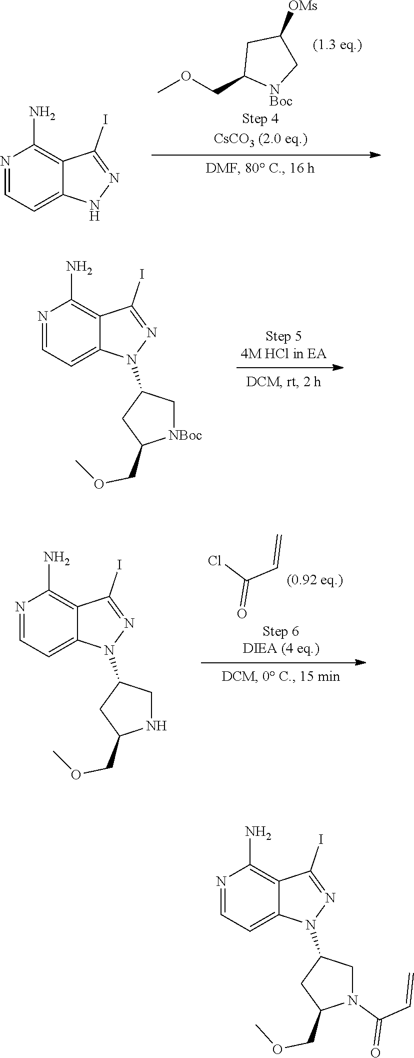

### Step 1: 4-chloro-3-iodo-1H-pyrazolo[4,3-c]pyridine

To a solution of 4-chloro-1H-pyrazolo[4,3-c]pyridine (5.00 g, 32.56 mmol) in DMF (45 mL) was added NIS (14.65 g, 65.12 mmol). The reaction mixture was stirred for 2 d at room temperature under nitrogen atmosphere. The resulting mixture was diluted water (150 mL) at 0° C. The precipitated solids were collected by filtration, washed with sat. Na2S2O3 solution and dried to afford 4-chloro-3-iodo-1H-pyrazolo[4,3-c]pyridine (9.4 g, 96%) as an off-white solid which was used in the next step directly without further purification. MS ESI calculated for C6H3ClIN3 [M+H]+, 279.91, 281.90; found 279.95, 281.95.

### Step 2: N-[(2,4-dimethoxyphenyl)methyl]-3-iodo-1H-pyrazolo[4,3-c]pyridin-4-amine

To a stirred mixture of 4-chloro-3-iodo-1H-pyrazolo[4,3-c]pyridine (6.10 g, 21.83 mmol) in 1-butanol (120 mL) was added 1-(2,4-dimethoxyphenyl)methanamine (10.95 g, 65.48 mmol) at room temperature. The reaction mixture was stirred for 16 h at 110° C. under nitrogen atmosphere. The reaction mixture concentrated under reduced pressure. The residue was purified by silica gel chromatography, eluted with acetone in CHCl3 (0-14%). The fractions that contained desired product were concentrated to afford N-[(2,4-dimethoxyphenyl)methyl]-3-iodo-1H-pyrazolo[4,3-c]pyridin-4-amine (3.47 g, 38%) as an off-white solid. MS ESI calculated for C15H15IN4O2 [M+H]+, 411.02, found 411.05.

### Step 3: 3-iodo-1H-pyrazolo[4,3-c]pyridin-4-amine

A mixture of N-[(2,4-dimethoxyphenyl)methyl]-3-iodo-1H-pyrazolo[4,3-c]pyridin-4-amine (3.47 g, 8.46 mmol) in TFA (15 mL) was stirred for 2 h at 50° C. under nitrogen atmosphere. The resulting mixture was concentrated under vacuum. The residue was basified to pH 8 with saturated NaHCO₃(aq.) and the resulting mixture was extracted with EA (3×200 mL). The combined organic layers were washed brine (2×80 mL), dried over anhydrous Na2SO4 and filtered. The filtrate was concentrated under reduced pressure. The residue was purified by silica gel column chromatography, eluted with MeOH in CHCl3 (0-12%). The fractions that contained desired product were combined and concentrated to afford 3-iodo-1H-pyrazolo[4,3-c]pyridin-4-amine (2 g, 90%) as an off-white solid.

### Step 4: tert-butyl (2R)-4-[4-amino-3-iodopyrazolo[4,3-c]pyridin-1-yl]-2-(methoxymethyl)pyrrolidine-1-carboxylate

To a mixture of 3-iodo-1H-pyrazolo[4,3-c]pyridin-4-amine (2.50 g, 9.61 mmol) and Cs2CO3 (6.26 g, 19.21 mmol) in DMF (50 mL) was added tert-butyl (2R,4R)-4-(methanesulfonyloxy)-2-(methoxymethyl)pyrrolidine-1-carboxylate (4.07 g, 12.50 mmol). The reaction mixture was stirred for 16 h at 80° C. under nitrogen atmosphere. The resulting mixture was allowed to cool down to room temperature and diluted with water (50 mL). The resulting mixture was extracted with EA (3×150 mL). The combined organic layers were washed with brine (5×50 mL), dried over anhydrous Na2SO4 and filtered. The filtrate was concentrated under reduced pressure. The residue was purified by silica gel column chromatography, eluted with MeOH in DCM (0-8%). The fractions that contained desired product were combined and concentrated to afford tert-butyl (2R)-4-[4-amino-3-iodopyrazolo[4,3-c]pyridin-1-yl]-2-(methoxymethyl)pyrrolidine-1-carboxylate (4.92 g, 75%) as a brown oil. MS ESI calculated for C17H24IN5O3 [M+H]+, 474.09; found 474.20.

### Step 5: 3-iodo-1-[(5R)-5-(methoxymethyl)pyrrolidin-3-yl]pyrazolo[4,3-c]pyridin-4-amine

To a stirred solution of tert-butyl (2R)-4-[4-amino-3-iodopyrazolo[4,3-c]pyridin-1-yl]-2-(methoxymethyl)pyrrolidine-1-carboxylate (4.92 g, 6.24 mmol) in DCM (20.00 mL) was added 4 M HCl in EA (30.00 mL) dropwise at 0° C. under nitrogen atmosphere. The reaction mixture was stirred for 2 h at ambient temperature. The resulting mixture was concentrated under reduced pressure. The residue was purified by reverse flash chromatography with the following conditions: column, C18 silica gel; mobile phase, ACN in water (NH4HCO3 1 g/L), 10% to 35% gradient in 30 min; detector, UV 254 nm. The fractions that contained desired product were combined and concentrated to afford 3-iodo-1-[(5R)-5-(methoxymethyl)pyrrolidin-3-yl]pyrazolo[4,3-c]pyridin-4-amine (2.2 g, 81%) as an off-white solid. MS ESI calculated for C12H16IN5O [M+H]+, 374.04; found 374.05.

### Step 6: 1-((2R,4S)-4-(4-amino-3-iodo-1H-pyrazolo[4,3-c]pyridin-1-yl)-2-(methoxymethyl)pyrrolidin-1-yl)prop-2-en-1-one

To a stirred solution of 3-iodo-1-[(5R)-5-(methoxymethyl)pyrrolidin-3-yl]pyrazolo[4,3-c]pyridin-4-amine (1.00 g, 2.68 mmol) in DCM (3 mL) were added acryloyl chloride (21.83 mg, 0.241 mmol) and DIEA (1.87 mL, 10.736 mmol) dropwise at 0° C. The reaction mixture was stirred for 15 min at 0° C. The resulting mixture was diluted with water (20 mL) and extracted with DCM (3×20 mL). The combined organic layers were washed with brine (3×40 mL), dried over anhydrous Na2SO4 and filtered. The filtrate was concentrated under reduced pressure. The residue was purified by silica gel column chromatography, eluted with MeOH in DCM (0-8%). The fractions that contained desired product were combined and concentrated to afford 1-((2R,4S)-4-(4-amino-3-iodo-1H-pyrazolo[4,3-c]pyridin-1-yl)-2-(methoxymethyl)pyrrolidin-1-yl)prop-2-en-1-one (0.99 g, 86%) as an off-white solid. MS ESI calculated for C15H18IN5O2 [M+H]+, 428.05; found 428.10.

### Intermediate 4: (2R,4S)-tert-butyl 4-(4-amino-3-iodo-1H-pyrazolo[3,4-d]pyrimidin-1-yl)-2-(methoxymethyl)pyrrolidine-1-carboxylate

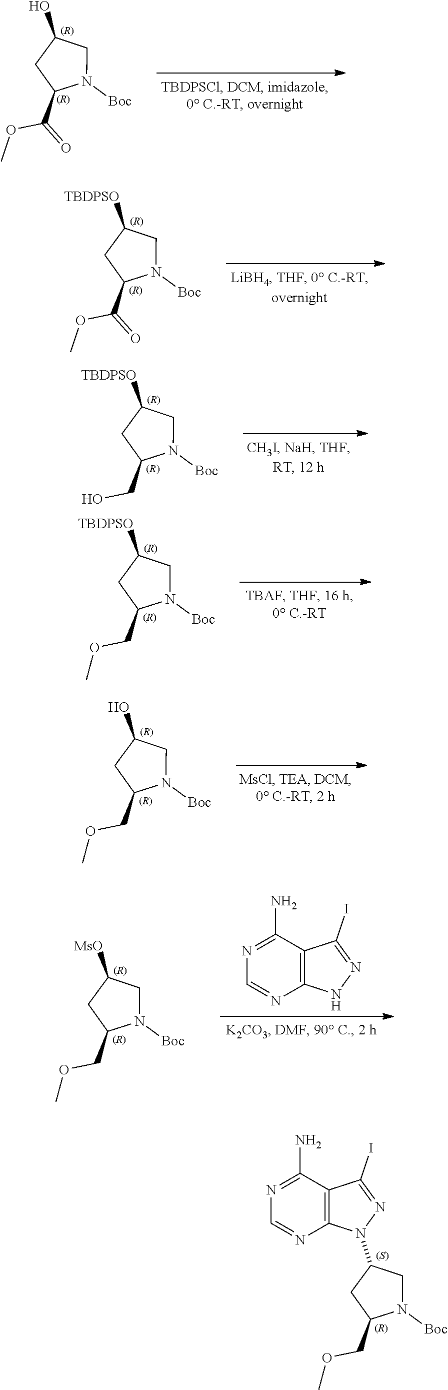

### Step 1: (2R,4R)-1-tert-butyl 2-methyl 4-((tert-butyldiphenylsilyl)oxy)pyrrolidine-1,2-dicarboxylate

To a mixture of (2R,4R)-1-tert-butyl 2-methyl 4-hydroxypyrrolidine-1,2-dicarboxylate (100.0 g, 408.2 mmol) and imidazole (39.0 g, 573.5 mmol) in DCM (1.5 L) was added TBDPSCl (123.0 g, 448.9 mmol) at 0° C. The reaction mixture was stirred at room temperature overnight. The reaction mixture was quenched with water (1.5 L) and extracted with DCM (1.0 L) twice. The combined organic layers were washed with brine (1.5 L), dried over Na2SO4, filtered and concentrated. The residue was triturated with PE (1.5 L) to afford (2R,4R)-1-tert-butyl 2-methyl 4-((tert-butyldiphenylsilyl)oxy)pyrrolidine-1,2-dicarboxylate (120.0 g, 60%) as a white solid. [M+H] Calcd.: 484.2; Found, 484.2.

### Step 2: (2R,4R)-tert-butyl 4-((tert-butyldiphenylsilyl)oxy)-2-(hydroxymethyl)pyrrolidine-1-carboxylate

To a mixture of (2R,4R)-1-tert-butyl 2-methyl 4-((tert-butyldiphenylsilyl)oxy)pyrrolidine-1,2-dicarboxylate (10.0 g, 20.7 mmol) in THF (150 mL) was added 2 M LiBH4 in THF (55 mL, 110 mmol) at 0° C. The reaction mixture was stirred at room temperature overnight. The reaction mixture was quenched with water (1.0 L) and extracted with EA (1.0 L) twice. The combined organic layers were washed with brine (1.0 L), dried over Na2SO4, filtered and concentrated to give a residue which was purified by silica gel column chromatography (PE/EA=4/1) to afford (2R,4R)-tert-butyl 4-((tert-butyldiphenylsilyl)oxy)-2-(hydroxymethyl)pyrrolidine-1-carboxylate (9.6 g, 100%) as yellow oil. [M+H] Calcd.: 456.2; Found, 456.2.

### Step 3: (2R,4R)-tert-butyl 4-((tert-butyldiphenylsilyl)oxy)-2-(methoxymethyl)pyrrolidine-1-carboxylate

To a mixture of (2R,4R)-tert-butyl 4-((tert-butyldiphenylsilyl)oxy)-2-(hydroxymethyl)pyrrolidine-1-carboxylate (9.6 g, 20.7 mmol) in THF (100 mL) was added NaH (1.3 g, 60%, 32.5 mmol). The reaction mixture was stirred at room temperature for 30 min and then added CH3I (6.0 g, 42.6 mmol). The reaction mixture was stirred at room temperature for 12 h. The reaction mixture was quenched with water (1.0 L) and extracted with EA (1.0 L) twice. The combined organic layers were washed with brine (1.0 L), dried over Na2SO4, filtered and concentrated to give a residue which was purified by silica gel column chromatography (PE/EA=4/1) to afford (2R,4R)-tert-butyl 4-((tert-butyldiphenylsilyl)oxy)-2-(methoxymethyl)pyrrolidine-1-carboxylate (7.8 g, 81%) as a white solid. [M+H] Calcd.: 470.3; Found, 470.3.

### Step 4: (2R,4R)-tert-butyl 4-hydroxy-2-(methoxymethyl)pyrrolidine-1-carboxylate

To a mixture of (2R,4R)-tert-butyl 4-((tert-butyldiphenylsilyl)oxy)-2-(methoxymethyl)pyrrolidine-1-carboxylate (20.0 g, 42.6 mmol) in THF (200 mL) was added 1 M TBAF in THF (64.0 mL, 64.0 mmol). The reaction mixture was stirred at room temperature for 16 h. The reaction mixture was quenched with water (1.0 L) and extracted with EA (1.0 L) twice. The combined organic layers were washed with brine (1.0 L), dried over Na2SO4, filtered and concentrated to give a residue which was purified by silica gel column chromatography (PE/EA=2/1) to afford (2R,4R)-tert-butyl 4-hydroxy-2-(methoxymethyl)pyrrolidine-1-carboxylate (5.0 g, 50%) as colorless oil.

### Step 5: (2R,4R)-tert-butyl 2-(methoxymethyl)-4-((methylsulfonyl)oxy)pyrrolidine-1-carboxylate

To a mixture of (2R,4R)-tert-butyl 4-hydroxy-2-(methoxymethyl)pyrrolidine-1-carboxylate (3.4 g, 14.7 mmol) and TEA (4.4 g, 44.1 mmol) in DCM (50 mL) was added MsCl (3.4 g, 29.4 mmol) at 0° C. The reaction mixture was stirred at room temperature for 2 h. The reaction mixture was quenched with ice water (100 mL) and extracted with DCM (100 mL) for three times. The combined organic layers were washed with brine, dried over sodium sulfate, filtered and concentrated to afford the crude (2R,4R)-tert-butyl 2-(methoxymethyl)-4-((methylsulfonyl)oxy)pyrrolidine-1-carboxylate (3.4 g, 100%, crude) as yellow oil.

### Step 6: (2R,4S)-tert-butyl 4-(4-amino-3-iodo-1H-pyrazolo[3,4-d]pyrimidin-1-yl)-2-(methoxymethyl)pyrrolidine-1-carboxylate

To a solution of 3-iodo-1H-pyrazolo[3,4-d]pyrimidin-4-amine (4.8 g, 14.7 mmol) and crude (2R,4R)-tert-butyl 2-(methoxymethyl)-4-((methylsulfonyl)oxy)pyrrolidine-1-carboxylate (3.4 g, 14.7 mmol) in DMF (50 mL) was added K2CO3 (6.0 g, 34.9 mmol). The reaction mixture was stirred at 90° C. for 2 h. The reaction mixture was quenched with ice water (100 mL) and extracted with DCM (100 mL) for three times. The combined organic layers were washed with brine, dried over sodium sulfate, filtered and concentrated to afford a residue which was purified by silica gel column chromatography (DCM/MeOH=30/1) to afford (2R,4S)-tert-butyl 4-(4-amino-3-iodo-1H-pyrazolo[3,4-d]pyrimidin-1-yl)-2-(methoxymethyl)pyrrolidine-1-carboxylate (3.7 g, 53%) as a yellow solid. [M+H] Calcd.: 475.1; Found, 475.1.

### Intermediate 5: (2R,4S)-tert-butyl 4-(4-amino-3-iodo-1H-pyrazolo[4,3-c]pyridin-1-yl)-2-(methoxymethyl)pyrrolidine-1-carboxylate

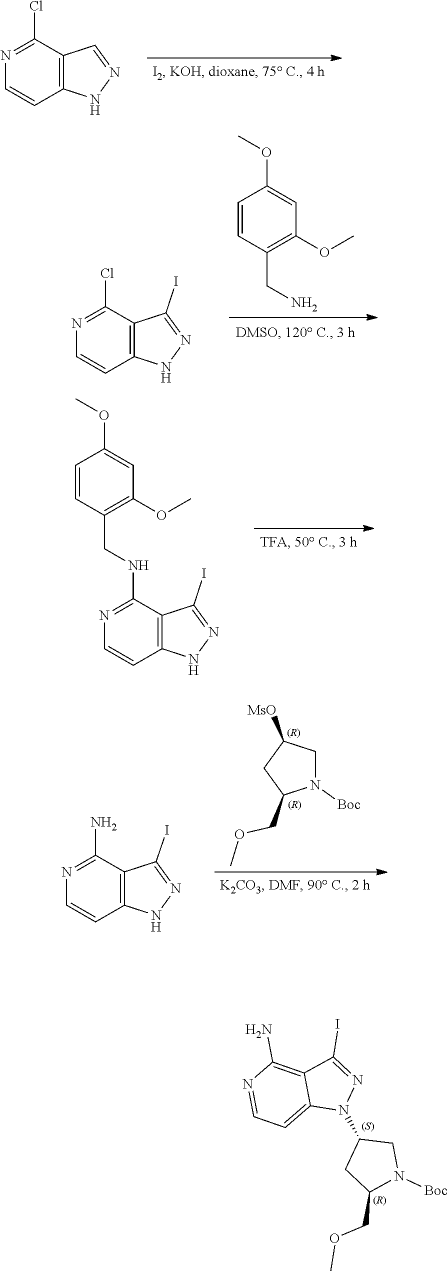

### Step 1: 4-chloro-3-iodo-1H-pyrazolo[4,3-c]pyridine

A solution of 4-chloro-1H-pyrazolo[4,3-c]pyridine (1.5 g, 9.8 mmol), 1,4-dioxane (25 mL), potassium hydroxide (2.0 g, 35.7 mmol) and iodine (4.9 g, 19.5 mmol) was stuffed for 4 h at 75° C. The reaction mixture was quenched with saturated aqueous sodium thiosulfate pentahydrate (30 mL) and the solids were collected by filtration to give 4-chloro-3-iodo-1H-pyrazolo[4,3-c]pyridine (2.5 g, 92%) as a yellow solid. [M+H] Calcd.: 280.0; Found, 280.0.

### Step 2: N-(2,4-dimethoxybenzyl)-3-iodo-1H-pyrazolo[4,3-c]pyridin-4-amine

To a solution of 4-chloro-3-iodo-1H-pyrazolo[4,3-c]pyridine (8.5 g, 30.0 mmol) in DMSO (100 mL) was added (2,4-dimethoxyphenyl)methanamine (15.3 g, 90.0 mmol). The mixture was heated at 120° C. for 3 h. The mixture was cooled to rt, diluted EA (200 mL), washed with water (100 mL) and brine (100 mL), dried over Na2SO4, filtered and concentrated to afford N-(2,4-dimethoxybenzyl)-3-iodo-1H-pyrazolo[4,3-c]pyridin-4-amine (8.6 g, 70%) as a yellow oil which was used for next step without further purification. [M+H] Calcd.: 411.0; Found, 411.0.

### Step 3: 3-iodo-1H-pyrazolo[4,3-c]pyridin-4-amine

A mixture of N-(2,4-dimethoxybenzyl)-3-iodo-1H-pyrazolo[4,3-c]pyridin-4-amine (8.6 g, 21.0 mmol) in TFA (50 mL) was heated to 50° C. for 3 h. After the solvent was removed, the residue was basified to pH=8 with sodium bicarbonate aqueous solution and extracted with EA (100 mL) for three times. The combined organic layers were washed with brine (100 mL), dried over anhydrous sodium sulfate, filtered and evaporated under vacuum. The residue was purified by column chromatography (PE/EA=3/1) to afford 3-iodo-1H-pyrazolo[4,3-c]pyridin-4-amine (4.3 g, 60%) as a white solid. [M+H] Calcd.: 260.7; Found, 260.7.

### Step 4: (2R,4S)-tert-butyl 4-(4-amino-3-iodo-1H-pyrazolo[4,3-c]pyridin-1-yl)-2-(methoxymethyl)pyrrolidine-1-carboxylate

To a solution of 3-iodo-1H-pyrazolo[4,3-c]pyridin-4-amine (3.8 g, 14.7 mmol) and crude (2R,4R)-tert-butyl 2-(methoxymethyl)-4-((methylsulfonyl)oxy)pyrrolidine-1-carboxylate (3.4 g, 14.7 mmol) in DMF (50 mL) was added K2CO3 (6.0 g, 34.9 mmol). The reaction mixture was stirred at 90° C. for 2 h. The reaction mixture was cooled to rt, quenched with ice water (100 mL) and extracted with DCM (100 mL) for three times. The combined organic layers were washed with brine, dried over sodium sulfate, filtered and concentrated to afford a residue which was purified by silica gel column chromatography (DCM/MeOH=30/1) to afford (2R,4S)-tert-butyl 4-(4-amino-3-iodo-1H-pyrazolo[4,3-c]pyridin-1-yl)-2-(methoxymethyl)pyrrolidine-1-carboxylate (3.4 g, 50%) as a yellow solid. [M+H] Calcd.: 474.1; Found, 474.1.

### Intermediate 6: (2R,4S)-tert-butyl 4-(4-chloro-3-iodo-1H-pyrrolo[3,2-c]pyridin-1-yl)-2-(methoxymethyl)pyrrolidine-1-carboxylate

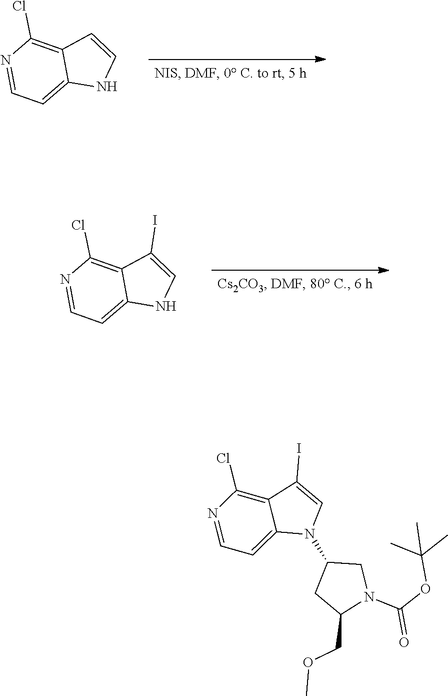 [SMILES: Clc1nccc2[nH]ccc12 ; Clc1nccc2[nH]cc(I)c12 ; COC[C@H]1C[C@H](n2cc(I)c3c(Cl)nccc32)CN1C(=O)OC(C)(C)C]

### Step 1: 4-chloro-3-iodo-1H-pyrrolo[3,2-c]pyridine

To a solution of 4-chloro-1H-pyrrolo[3,2-c]pyridine (10.0 g, 65.6 mmol) in DMF (50 mL) was added NIS (22.2 g, 98.3 mmol) at 0° C., then the mixture was warmed to rt and stirred for 5 h. The reaction mixture was quenched with saturated aqueous sodium thiosulfate pentahydrate (50 mL) and the solids were collected by filtration to afford 4-chloro-3-iodo-1H-pyrrolo[3,2-c]pyridine (16.0 g, 87%) as a white solid. [M+H] Calcd.: 278.9; Found, 278.9.

### Step 2: (2R,4S)-tert-butyl 4-(4-chloro-3-iodo-1H-pyrrolo[3,2-c]pyridin-1-yl)-2-(methoxymethyl)pyrrolidine-1-carboxylate

To a solution of 4-chloro-1H-pyrrolo[3,2-c]pyridine (5.0 g, 18.0 mmol) and crude (2R,4R)-tert-butyl 2-(methoxymethyl)-4-((methylsulfonyl)oxy)pyrrolidine-1-carboxylate (7.1 g, 23.0 mmol) in DMF (50 mL) was added Cs2CO3 (11.7 g, 35.8 mmol) at rt. The reaction mixture was stirred at 80° C. for 6 h. The reaction mixture was cooled to rt, quenched with ice water (100 mL) and extracted with DCM (100 mL) for three times. The combined organic layers were washed with brine, dried over sodium sulfate, filtered and concentrated to afford a residue which was purified by silica gel column chromatography (DCM/MeOH=30/1) to afford (2R,4S)-tert-butyl 4-(4-chloro-3-iodo-1H-pyrrolo[3,2-c]pyridin-1-yl)-2-(methoxymethyl)pyrrolidine-1-carboxylate (4.5 g, 51%) as a white solid. [M+H] Calcd.: 492.1; Found, 492.1.

### Example 1: 1-((2R,4S)-4-(4-amino-3-((2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

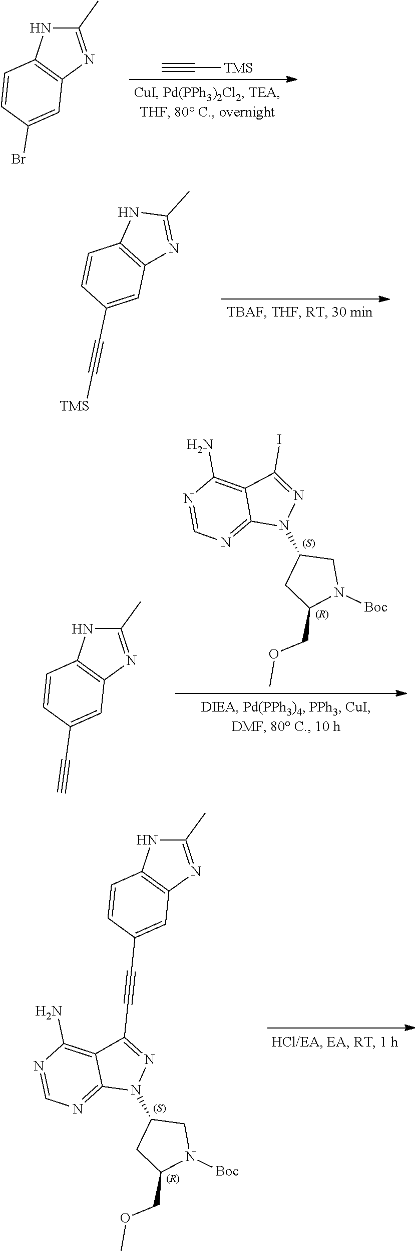

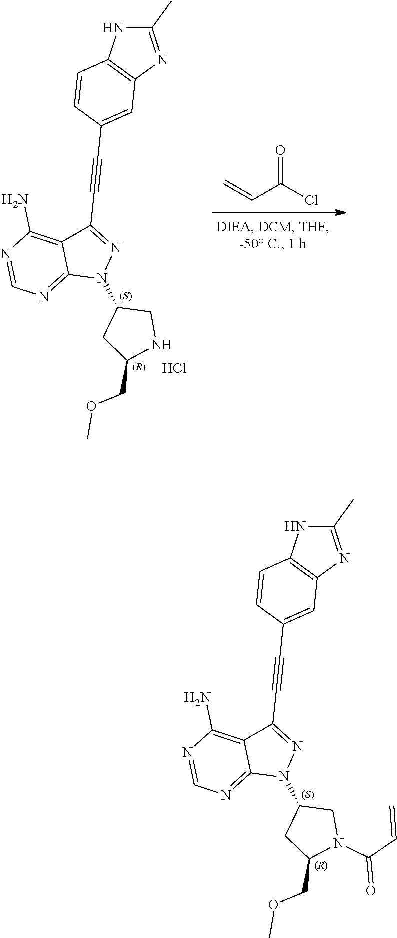

### Step 1: 2-methyl-5-((trimethylsilyl)ethynyl)-1H-benzo[d]imidazole

To a solution of 5-bromo-2-methyl-1H-benzo[d]imidazole (2.0 g, 9.5 mmol) and CuI (200 mg, 1.05 mmol), Pd(PPh3)2Cl2 (532 mg, 0.76 mmol) in THF (60 mL) and TEA (30 mL) was added ethynyltrimethylsilane (5.6 g, 56.9 mmol). The reaction mixture was stirred at 80° C. overnight under nitrogen atmosphere. After cooling down to room temperature, the solvent was removed to give a residue which was purified by flash (PE/EA=1/3) to afford 2-methyl-5-((trimethylsilyl)ethynyl)-1H-benzo[d]imidazole (400 mg, 18%) as a yellow solid. [M+H] Calcd.: 229.1; Found, 229.1.

### Step 2: 5-ethynyl-2-methyl-1H-benzo[d]imidazole

To a solution of 2-methyl-5-((trimethylsilyl)ethynyl)-1H-benzo[d]imidazole (400 mg, 1.75 mmol) in THF (20 mL) was added 1 M TBAF in THF (2.40 mL, 2.40 mmol). The reaction mixture was stirred at room temperature for 30 min. The solvent was removed to give a residue which was purified by flash (DCM/MeOH=20/1) to afford 5-ethynyl-2-methyl-1H-benzo[d]imidazole (210 mg, 77%) as a yellow solid. [M+H] Calcd.: 157.1; Found, 157.1.

### Step 3: (2R,4S)-tert-butyl 4-(4-amino-3-((2-methyl-1H-benzo[d]imidazol-5-yl)ethynyl)-1H-pyrazolo[3,4-d]pyrimidin-1-yl)-2-(methoxymethyl)pyrrolidine-1-carboxylate

To a mixture of (2R,4S)-tert-butyl 4-(4-amino-3-iodo-1H-pyrazolo[3,4-d]pyrimidin-1-yl)-2-(methoxymethyl)pyrrolidine-1-carboxylate (303 mg, 0.64 mmol), 5-ethynyl-2-methyl-1H-benzo[d]imidazole (100 mg, 0.64 mmol) and CuI (11 mg, 0.06 mmol) in DMF (10 mL) was added Pd(PPh3)4 (44 mg, 0.04 mmol), PPh3 (10 mg, 0.04 mmol) and DIEA (248 mg, 1.92 mmol) under nitrogen atmosphere and stirred at 80° C. for 10 h. After cooling down to room temperature. The reaction mixture was cooled to rt, diluted with water (20 mL) and extracted with DCM (20 mL) for three times, the combined organic layers were dried over Na2SO4, filtered and concentrated in vacuo to give a residue, which was purified by flash (100% EA) to afford (2R,4S)-tert-butyl 4-(4-amino-3-((2-methyl-1H-benzo[d]imidazol-5-yl)ethynyl)-1H-pyrazolo[3,4-d]pyrimidin-1-yl)-2-(methoxymethyl)pyrrolidine-1-carboxylate (160 mg, 50%) as a yellow solid. [M+H] Calcd.: 503.2; Found, 503.2.

### Step 4: 1-((3S,5R)-5-(methoxymethyl)pyrrolidin-3-yl)-3-((2-methyl-1H-benzo[d]imidazol-5-yl)ethynyl)-1H-pyrazolo[3,4-d]pyrimidin-4-amine hydrochloride

To a solution of (2R,4S)-tert-butyl 4-(4-amino-3-((2-methyl-1H-benzo[d]imidazol-5-yl)ethynyl)-1H-pyrazolo[3,4-d]pyrimidin-1-yl)-2-(methoxymethyl)pyrrolidine-1-carboxylate (160 mg, 0.32 mmol) in EA (10 mL) was added HC/EA (10 mL). The reaction mixture was stirred at room temperature for 1 h. The reaction mixture was concentrated in vacuo to afford 1-((3S,5R)-5-(methoxymethyl)pyrrolidin-3-yl)-3-((2-methyl-1H-benzo[d]imidazol-5-yl)ethynyl)-1H-pyrazolo[3,4-d]pyrimidin-4-amine hydrochloride (128 mg, 100%) as a yellow solid. [M+H] Calcd.: 403.2; Found, 403.2.

### Step 5: 1-((2R,4S)-4-(4-amino-3-((2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of 1-((3S,5R)-5-(methoxymethyl)pyrrolidin-3-yl)-3-((2-methyl-1H-benzo[d]imidazol-5-yl)ethynyl)-1H-pyrazolo[3,4-d]pyrimidin-4-amine hydrochloride (128 mg, 0.32 mmol) and DIEA (124 mg, 0.96 mmol) in DCM (10 mL) and THF (10 mL) at −50° C. under nitrogen atmosphere was added a solution of acryloyl chloride (29 mg, 0.32 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (64.8 mg, 44%) as a white solid. 1H NMR (400 MHz, CDCl3): 8.37-8.34 (m, 1H), 7.76-7.74 (m, 1H), 7.53-7.40 (m, 2H), 6.65-6.39 (m, 2H), 5.95 (br s, 2H), 5.83-5.67 (m, 2H), 4.66-4.53 (m, 1H), 4.22-4.04 (m, 2H), 3.84-3.80 (m, 1H), 3.55-3.51 (m, 1H), 3.40-3.39 (m, 3H), 2.66 (s, 3H), 2.51-2.46 (m, 2H). [M+H] Calcd.: 457.2; Found, 457.2.

### Example 2: 1-((2R,4S)-4-(3-((1H-indazol-5-yl)ethynyl)-4-amino-1H-pyrazolo[3,4-d]pyrimidin-1-yl)-2-(methoxymethyl)pyrrolidin-1-yl)prop-2-en-1-one

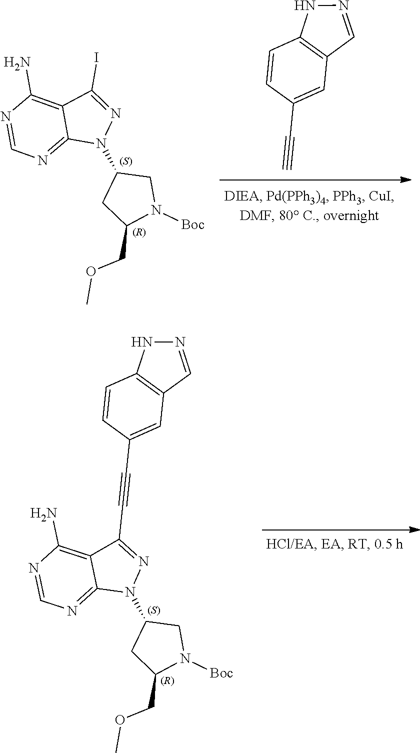

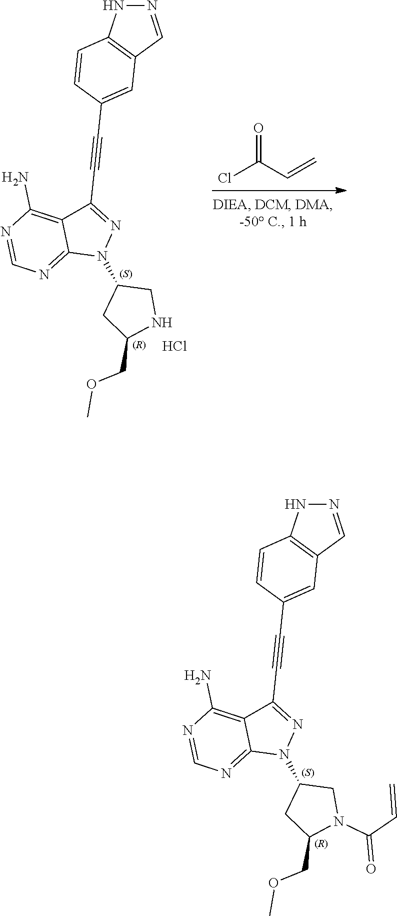

### Step 1: (2R,4S)-tert-butyl 4-(3-((1H-indazol-5-yl)ethynyl)-4-amino-1H-pyrazolo[3,4-d]pyrimidin-1-yl)-2-(methoxymethyl)pyrrolidine-1-carboxylate

To a mixture of (2R,4S)-tert-butyl 4-(4-amino-3-iodo-1H-pyrazolo[3,4-d]pyrimidin-1-yl)-2-(methoxymethyl)pyrrolidine-1-carboxylate (1.0 g, 2.1 mmol), 5-ethynyl-1H-indazole (300 mg, 2.11 mmol) and CuI (60 mg, 0.32 mmol) in DMF (20 mL) was added Pd(PPh3)₄ (122 mg, 0.11 mmol), PPh3 (28 mg, 0.11 mmol) and DIEA (816 mg, 6.33 mmol) under nitrogen atmosphere and stirred at 80° C. overnight. After cooling down to room temperature. The reaction mixture was cooled to rt, diluted with water (50 mL) and extracted with DCM (50 mL) for three times, the combined organic layers were dried over Na2SO4, filtered and concentrated in vacuo to give a residue which was purified by flash (DCM/MeOH=10/1) to afford (2R,4S)-tert-butyl 4-(3-((1H-indazol-5-yl)ethynyl)-4-amino-1H-pyrazolo[3,4-d]pyrimidin-1-yl)-2-(methoxymethyl)pyrrolidine-1-carboxylate (1.0 g, 97%) as a yellow solid. [M+H] Calcd.: 489.2; Found, 489.2.

### Step 2: 3-((1H-indazol-5-yl)ethynyl)-1-((3S,5R)-5-(methoxymethyl)pyrrolidin-3-yl)-1H-pyrazolo[3,4-d]pyrimidin-4-amine hydrochloride

To a solution of (2R,4S)-tert-butyl 4-(3-((1H-indazol-5-yl)ethynyl)-4-amino-1H-pyrazolo[3,4-d]pyrimidin-1-yl)-2-(methoxymethyl)pyrrolidine-1-carboxylate (300 mg, 0.61 mmol) in EA (40 mL) was added HCl/EA (2 mL). The reaction mixture was stirred at room temperature for 30 min. The reaction mixture was concentrated in vacuo to afford 3-((1H-indazol-5-yl)ethynyl)-1-((3S,5R)-5-(methoxymethyl)pyrrolidin-3-yl)-1H-pyrazolo[3,4-d]pyrimidin-4-amine hydrochloride (250 mg, 97%) as a yellow solid. [M+H] Calcd.: 389.2; Found, 389.2.

### Step 3: 1-((2R,4S)-4-(3-((1H-indazol-5-yl)ethynyl)-4-amino-1H-pyrazolo[3,4-d]pyrimidin-1-yl)-2-(methoxymethyl)pyrrolidin-1-yl)prop-2-en-1-one

To a solution of 3-((1H-indazol-5-yl)ethynyl)-1-((3S,5R)-5-(methoxymethyl)pyrrolidin-3-yl)-1H-pyrazolo[3,4-d]pyrimidin-4-amine hydrochloride (250 mg, 0.59 mmol) and DIEA (230 mg, 1.77 mmol) in DCM (4 mL) and DMA (4 mL) at −50° C. under nitrogen atmosphere was added a solution of acryloyl chloride (53 mg, 0.59 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(3-((1H-indazol-5-yl)ethynyl)-4-amino-1H-pyrazolo[3,4-d]pyrimidin-1-yl)-2-(methoxymethyl)pyrrolidin-1-yl)prop-2-en-1-one (109.0 mg, 42%) as a white solid. 1H NMR (400 MHz, DMSO-d6): 13.32 (s, 1H), 8.27-8.16 (m, 3H), 7.65-7.60 (m, 2H), 6.78-6.53 (m, 1H), 6.20-6.14 (m, 1H), 5.69-5.62 (m, 2H), 4.59-4.46 (m, 1H), 4.08-3.81 (m, 2H), 3.64-3.47 (m, 2H), 3.33 (s, 3H), 2.67-2.55 (m, 1H), 2.50-2.37 (m, 1H). [M+H] Calcd.: 443.2; Found, 443.2.

### Example 3: 1-((2R,4S)-4-(4-amino-3-((1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

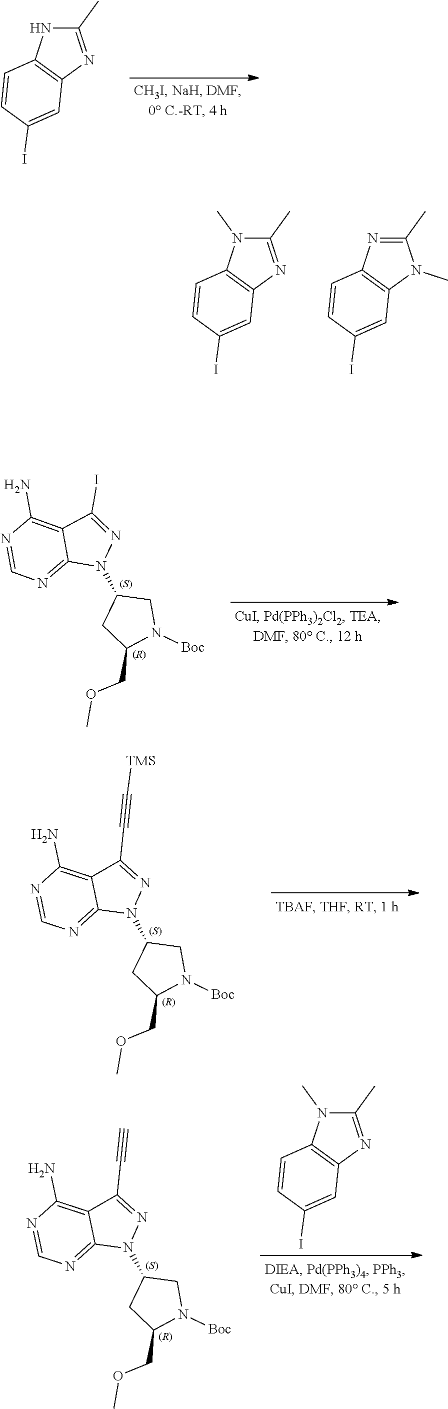

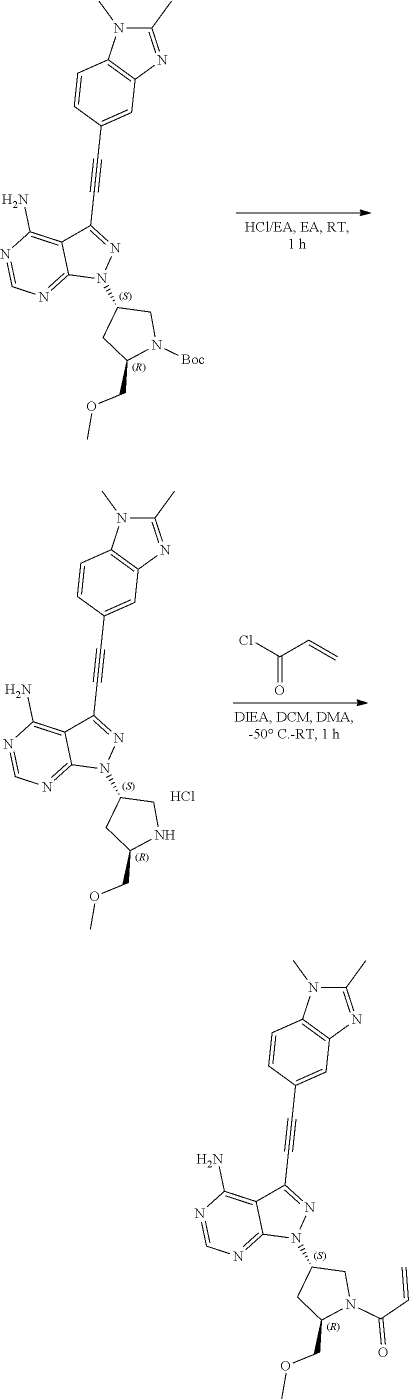

### Step 1: 5-iodo-1,2-dimethyl-1H-benzo[d]imidazole & 6-iodo-1,2-dimethyl-1H-benzo[d]imidazole

To a mixture of 5-iodo-2-methyl-1H-benzo[d]imidazole (2.0 g, 7.8 mmol) in DMF (30 mL) was added NaH (322 mg, 8.05 mmol, 60% wt.) at 0° C. The reaction mixture was stirred at 0° C. for 30 min, then added CH3I (1.3 g, 9.2 mmol). The reaction mixture was stirred at room temperature for 4 h. The reaction mixture was quenched with water (50 mL) and extracted with EA (50 mL) twice. The combined organic layers were washed with brine (50 mL), dried over Na2SO4, filtered and concentrated to give a residue, which was purified by silica gel column chromatography (DCM/MeOH=25/1) to afford 5-iodo-1,2-dimethyl-1H-benzo[d]imidazole (270 mg, 13%) and 6-iodo-1,2-dimethyl-1H-benzo[d]imidazole (370 mg, 17%) as a yellow solid. [M+H] Calcd.: 273.0; Found, 273.0.

### Step 2: (2R,4S)-tert-butyl 4-(4-amino-3-((trimethylsilyl)ethynyl)-1H-pyrazolo[3,4-d]pyrimidin-1-yl)-2-(methoxymethyl)pyrrolidine-1-carboxylate

To a mixture of (2R,4S)-tert-butyl 4-(4-amino-3-iodo-1H-pyrazolo[3,4-d]pyrimidin-1-yl)-2-(methoxymethyl)pyrrolidine-1-carboxylate (5.0 g, 10.5 mmol) and CuI (190 mg, 1.00 mmol), Pd(PPh3)2Cl2 (702 mg, 1.00 mmol) in DMF (60 mL) was added ethynyltrimethylsilane (6.2 g, 63.0 mmol) and TEA (5.3 g, 52.8 mmol). The reaction mixture was stirred at 80° C. for 12 h under nitrogen atmosphere. After cooling down to room temperature, the solvent was removed to give a residue, which was purified by flash (PE/EA=1/3) to afford (2R,4S)-tert-butyl 4-(4-amino-3-((trimethylsilyl)ethynyl)-1H-pyrazolo[3,4-d]pyrimidin-1-yl)-2-(methoxymethyl)pyrrolidine-1-carboxylate (2.5 g, 54%) as a white solid. [M+H] Calcd.: 445.2; Found, 445.2.

### Step 3: (2R,4S)-tert-butyl 4-(4-amino-3-ethynyl-1H-pyrazolo[3,4-d]pyrimidin-1-yl)-2-(methoxymethyl)pyrrolidine-1-carboxylate

To a solution of (2R,4S)-tert-butyl 4-(4-amino-3-((trimethylsilyl)ethynyl)-1H-pyrazolo[3,4-d]pyrimidin-1-yl)-2-(methoxymethyl)pyrrolidine-1-carboxylate (1.0 g, 2.3 mmol) in THF (20 mL) was added 1 M TBAF in THF (3.0 mL, 3.0 mmol). The reaction mixture was stirred at room temperature for 1 h. The reaction mixture was quenched with water (30 mL) and extracted with DCM (30 mL) for three times. The combined organic layers were dried over Na2SO4, filtered and concentrated to give a residue, which was purified by flash (PE/EA=1/1) to afford (2R,4S)-tert-butyl 4-(4-amino-3-ethynyl-1H-pyrazolo[3,4-d]pyrimidin-1-yl)-2-(methoxymethyl)pyrrolidine-1-carboxylate (500 mg, 61%) as a white solid. [M+H] Calcd.: 373.2; Found, 373.2.

### Step 4: (2R,4S)-tert-butyl 4-(4-amino-3-((1,2-dimethyl-1H-benzo[d]imidazol-5-yl)ethynyl)-1H-pyrazolo[3,4-d]pyrimidin-1-yl)-2-(methoxymethyl)pyrrolidine-1-carboxylate

To a mixture of (2R,4S)-tert-butyl 4-(4-amino-3-ethynyl-1H-pyrazolo[3,4-d]pyrimidin-1-yl)-2-(methoxymethyl)pyrrolidine-1-carboxylate (223 mg, 0.60 mmol), 5-iodo-1,2-dimethyl-1H-benzo[d]imidazole (136 mg, 0.50 mmol) and CuI (14 mg, 0.07 mmol) in DMF (15 mL) was added Pd(PPh3)4 (29 mg, 0.03 mmol), PPh3 (7 mg, 0.03 mmol) and DIEA (193 mg, 1.50 mmol) under nitrogen atmosphere and stirred at 80° C. for 5 h. After cooling down to room temperature. The reaction mixture was cooled to rt, diluted with water (30 mL) and extracted with DCM (30 mL) for three times. The combined organic layers were dried over Na2SO4, filtered and concentrated in vacuo to give a residue which was purified by flash (DCM/MeOH=10/1) to afford (2R,4S)-tert-butyl 4-(4-amino-3-((1,2-dimethyl-1H-benzo[d]imidazol-5-yl)ethynyl)-1H-pyrazolo[3,4-d]pyrimidin-1-yl)-2-(methoxymethyl)pyrrolidine-1-carboxylate (150 mg, 58%) as a yellow solid. [M+H] Calcd.: 517.3; Found, 517.3.

### Step 5: 3-((1,2-dimethyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine hydrochloride

To a solution of (2R,4S)-tert-butyl 4-(4-amino-3-((1,2-dimethyl-1H-benzo[d]imidazol-5-yl)ethynyl)-1H-pyrazolo[3,4-d]pyrimidin-1-yl)-2-(methoxymethyl)pyrrolidine-1-carboxylate (150 mg, 0.29 mmol) in EA (10 mL) was added HCl/EA (10 mL). The reaction mixture was stirred at room temperature for 1 h. The reaction mixture was concentrated in vacuo to afford 3-((1,2-dimethyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine hydrochloride (121 mg, 100%) as a yellow solid. [M+H] Calcd.: 417.2; Found, 417.2.

### Step 6: 1-((2R,4S)-4-(4-amino-3-((1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of 3-((1,2-dimethyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine hydrochloride (121 mg, 0.29 mmol) and DIEA (112 mg, 0.87 mmol) in DCM (20 mL) at −50° C. under nitrogen atmosphere was added a solution of acryloyl chloride (26 mg, 0.29 mmol) in DCM (0.5 mL) slowly. The mixture was stirred at −50° C. to room temperature for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (55.2 mg, 41%) as a yellow solid. 1H NMR (400 MHz, DMSO-d6): 8.26 (d, J=2.8 Hz, 1H), 7.93 (s, 1H), 7.57-7.51 (m, 2H), 6.78-6.71 (m, 1H), 6.60-6.53 (m, 1H), 6.19-6.14 (m, 1H), 5.71-5.58 (m, 2H), 4.59-4.46 (m, 1H), 4.11-3.83 (m, 2H), 3.80 (s, 3H), 3.64-3.47 (m, 2H), 3.33 (s, 3H), 2.67-2.55 (m, 4H), 2.38-2.35 (m, 1H). [M+H] Calcd.: 471.2; Found, 471.2.

### Example 4: 1-((2R,4S)-4-(4-amino-3-((1,2-dimethyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one

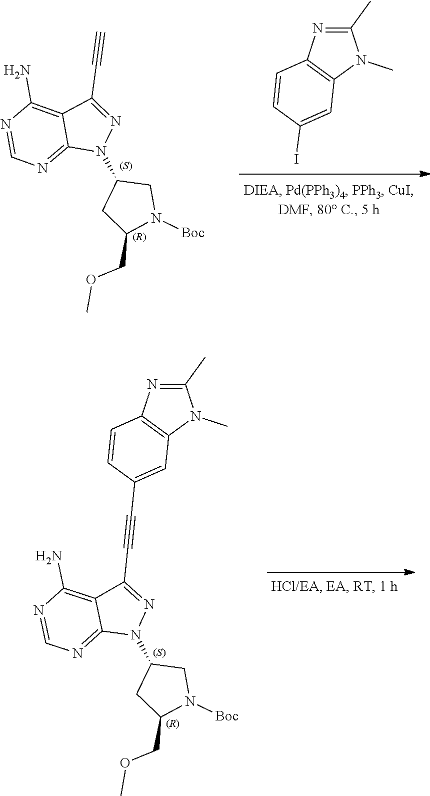

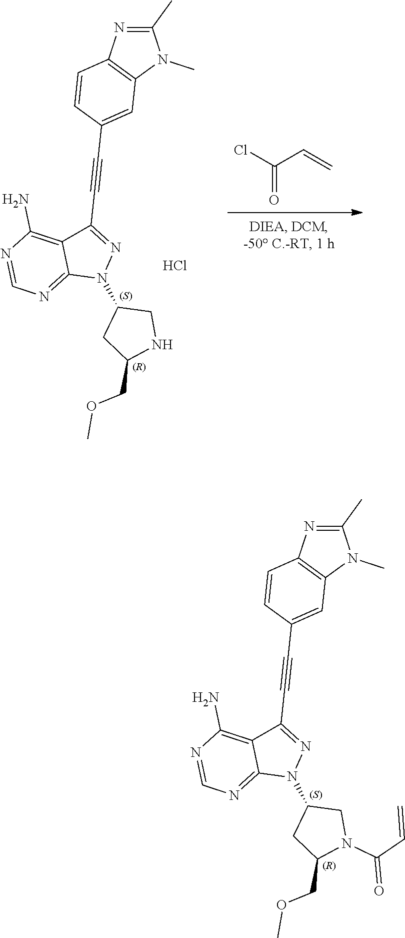

### Step 1: (2R,4S)-tert-butyl 4-(4-amino-3-((1,2-dimethyl-1H-benzo[d]imidazol-6-yl)ethynyl)-1H-pyrazolo[3,4-d]pyrimidin-1-yl)-2-(methoxymethyl)pyrrolidine-1-carboxylate

To a mixture of (2R,4S)-tert-butyl 4-(4-amino-3-ethynyl-1H-pyrazolo[3,4-d]pyrimidin-1-yl)-2-(methoxymethyl)pyrrolidine-1-carboxylate (262 mg, 0.72 mmol), 6-iodo-1,2-dimethyl-1H-benzo[d]imidazole (160 mg, 0.60 mmol) and CuI (17 mg, 0.09 mmol) in DMF (15 mL) was added Pd(PPh3)4 (35 mg, 0.06 mmol), PPh3 (8 mg, 0.06 mmol) and DIEA (232 mg, 1.80 mmol) under nitrogen atmosphere and stirred at 80° C. for 5 h. After cooling down to room temperature. The reaction mixture was cooled to rt, diluted with water (30 mL) and extracted with DCM (30 mL) for three times. The combined organic layers were dried over Na2SO4, filtered and concentrated in vacuo to give a residue which was purified by flash (DCM/MeOH=10/1) to afford (2R,4S)-tert-butyl 4-(4-amino-3-((1,2-dimethyl-1H-benzo[d]imidazol-6-yl)ethynyl)-1H-pyrazolo[3,4-d]pyrimidin-1-yl)-2-(methoxymethyl)pyrrolidine-1-carboxylate (230 mg, 58%) as a yellow solid. [M+H] Calcd.: 517.3; Found, 517.3.

### Step 2: 3-((1,2-dimethyl-1H-benzo[d]imidazol-6-yl)ethynyl)-1-((3S,5R)-5-(methoxymethyl)pyrrolidin-3-yl)-1H-pyrazolo[3,4-d]pyrimidin-4-amine hydrochloride

To a solution of (2R,4S)-tert-butyl 4-(4-amino-3-((1,2-dimethyl-1H-benzo[d]imidazol-6-yl)ethynyl)-1H-pyrazolo[3,4-d]pyrimidin-1-yl)-2-(methoxymethyl)pyrrolidine-1-carboxylate (230 mg, 0.44 mmol) in EA (10 mL) was added HCl/EA (10 mL). The reaction mixture was stirred at room temperature for 1 h. The reaction mixture was concentrated in vacuo to afford 3-((1,2-dimethyl-1H-benzo[d]imidazol-6-yl)ethynyl)-1-((3S,5R)-5-(methoxymethyl)pyrrolidin-3-yl)-1H-pyrazolo[3,4-d]pyrimidin-4-amine hydrochloride (185 mg, 100%) as a yellow solid. [M+H] Calcd.: 417.2; Found, 417.2.

### Step 3: 1-((2R,4S)-4-(4-amino-3-((1,2-dimethyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one

To a solution of 3-((1,2-dimethyl-1H-benzo[d]imidazol-6-yl)ethynyl)-1-((3S,5R)-5-(methoxymethyl)pyrrolidin-3-yl)-1H-pyrazolo[3,4-d]pyrimidin-4-amine hydrochloride (185 mg, 0.44 mmol) and DIEA (170 mg, 1.32 mmol) in DCM (20 mL) at −50° C. under nitrogen atmosphere was added a solution of acryloyl chloride (40 mg, 0.44 mmol) in DCM (0.5 mL) slowly. The mixture was stirred at −50° C. to room temperature for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((1,2-dimethyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one (82.3 mg, 40%) as a yellow solid. 1H NMR (400 MHz, DMSO-d6): 8.27 (d, J=2.4 Hz, 1H), 7.94 (s, 1H), 7.57-7.47 (m, 2H), 6.78-6.53 (m, 2H), 6.19-6.14 (m, 1H), 5.71-5.58 (m, 2H), 4.59-4.48 (m, 1H), 4.11-3.83 (m, 2H), 3.76 (s, 3H), 3.64-3.47 (m, 2H), 3.33 (s, 3H), 2.69-2.50 (m, 4H), 2.39-2.35 (m, 1H). [M+H] Calcd.: 471.2; Found, 471.2.

### Example 5: 1-((2R,4S)-4-(3-((1H-benzo[d]imidazol-5-yl)ethynyl)-4-amino-1H-pyrazolo[3,4-d]pyrimidin-1-yl)-2-(methoxymethyl)pyrrolidin-1-yl)prop-2-en-1-one

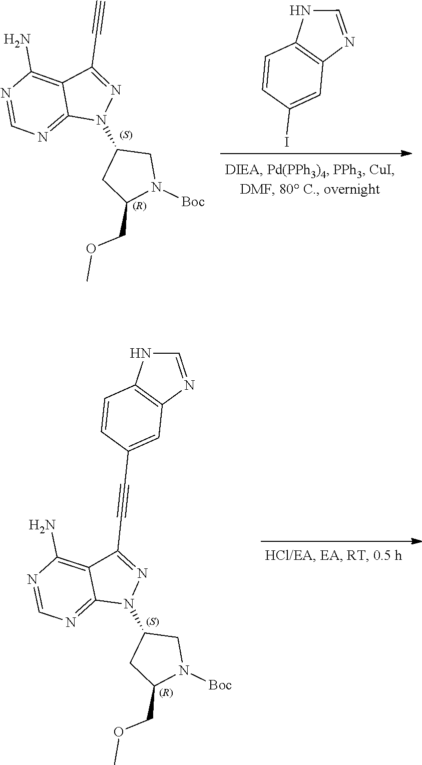

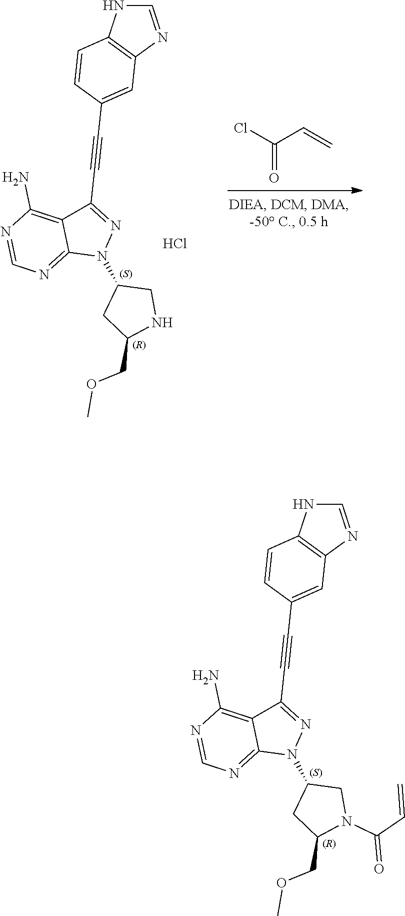

### Step 1: (2R,4S)-tert-butyl 4-(3-((1H-benzo[d]imidazol-5-yl)ethynyl)-4-amino-1H-pyrazolo[3,4-d]pyrimidin-1-yl)-2-(methoxymethyl)pyrrolidine-1-carboxylate

To a solution of (2R,4S)-tert-butyl 4-(4-amino-3-ethynyl-1H-pyrazolo[3,4-d]pyrimidin-1-yl)-2-(methoxymethyl)pyrrolidine-1-carboxylate (200 mg, 0.54 mmol), 5-iodo-1H-benzo[d]imidazole (224 mg, 0.65 mmol) and CuI (15 mg, 0.08 mmol) in DMF (15 mL) was added Pd(PPh3)4 (31 mg, 0.03 mmol), PPh3 (7 mg, 0.03 mmol) and DIEA (210 mg, 1.62 mmol) under nitrogen atmosphere and stirred at 80° C. overnight. The reaction mixture was cooled to rt, diluted with water (30 mL) and extracted with DCM (30 mL) for three times. The combined organic layers were dried over Na2SO4, filtered and concentrated in vacuo to give a residue which was purified by flash (DCM/MeOH=10/1) to afford (2R,4S)-tert-butyl 4-(3-((1H-benzo[d]imidazol-5-yl)ethynyl)-4-amino-1H-pyrazolo[3,4-d]pyrimidin-1-yl)-2-(methoxymethyl)pyrrolidine-1-carboxylate (185 mg, 71%) as a yellow solid. [M+H] Calcd.: 489.2; Found, 489.2.

### Step 2: 3-((1H-benzo[d]imidazol-5-yl)ethynyl)-1-((3S,5R)-5-(methoxymethyl)pyrrolidin-3-yl)-1H-pyrazolo[3,4-d]pyrimidin-4-amine hydrochloride

To a solution of (2R,4S)-tert-butyl 4-(3-((1H-benzo[d]imidazol-5-yl)ethynyl)-4-amino-1H-pyrazolo[3,4-d]pyrimidin-1-yl)-2-(methoxymethyl)pyrrolidine-1-carboxylate (180 mg, 0.37 mmol) in EA (10 mL) was added HCU/EA (10 mL). The mixture was stirred at room temperature for 0.5 h. The reaction mixture was concentrated in vacuo to afford 3-((1H-benzo[d]imidazol-5-yl)ethynyl)-1-((3S,5R)-5-(methoxymethyl)pyrrolidin-3-yl)-1H-pyrazolo[3,4-d]pyrimidin-4-amine hydrochloride (150 mg, 95%) as a yellow solid. [M+H] Calcd.: 389.2; Found, 389.2.

### Step 3: 1-((2R,4S)-4-(3-((1H-benzo[d]imidazol-5-yl)ethynyl)-4-amino-1H-pyrazolo[3,4-d]pyrimidin-1-yl)-2-(methoxymethyl)pyrrolidin-1-yl)prop-2-en-1-one

To a solution of 3-((1H-benzo[d]imidazol-5-yl)ethynyl)-1-((3S,5R)-5-(methoxymethyl)pyrrolidin-3-yl)-1H-pyrazolo[3,4-d]pyrimidin-4-amine hydrochloride (150 mg, 0.35 mmol) and DIEA (136 mg, 1.05 mmol) in DCM (1.5 mL) and DMA (1.5 mL) at −50° C. under nitrogen atmosphere was added a solution of acryloyl chloride (32 mg, 0.35 mmol) in DCM (0.5 mL) slowly. The mixture was stirred at −50° C. for 0.5 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(3-((1H-benzo[d]imidazol-5-yl)ethynyl)-4-amino-1H-pyrazolo[3,4-d]pyrimidin-1-yl)-2-(methoxymethyl)pyrrolidin-1-yl)prop-2-en-1-one (54.7 mg, 35%) as a yellow solid. 1H NMR (400 MHz, CDCl3): 8.36-8.35 (m, 1H), 8.20-8.13 (m, 1H), 7.94-7.91 (m, 1H), 7.71-7.68 (m, 1H), 7.51-7.50 (m, 1H), 6.60-6.45 (m, 2H), 6.36-6.11 (m, 1H), 5.81-5.73 (m, 2H), 4.68-4.51 (m, 1H), 4.15-4.07 (m, 1H), 3.83-3.82 (m, 1H), 3.54-3.52 (m, 2H), 3.39 (s, 3H), 2.97-2.75 (m, 1H), 2.51-2.49 (m, 1H). [M+H] Calcd.: 443.2; Found, 443.2.

### Example 6: 1-((2R,4S)-4-(4-amino-3-((1-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

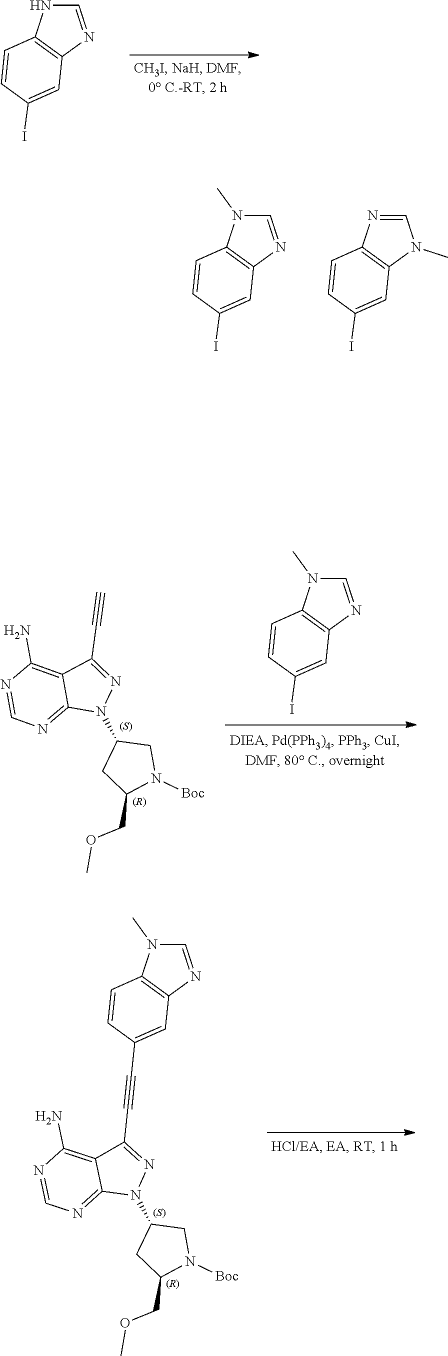

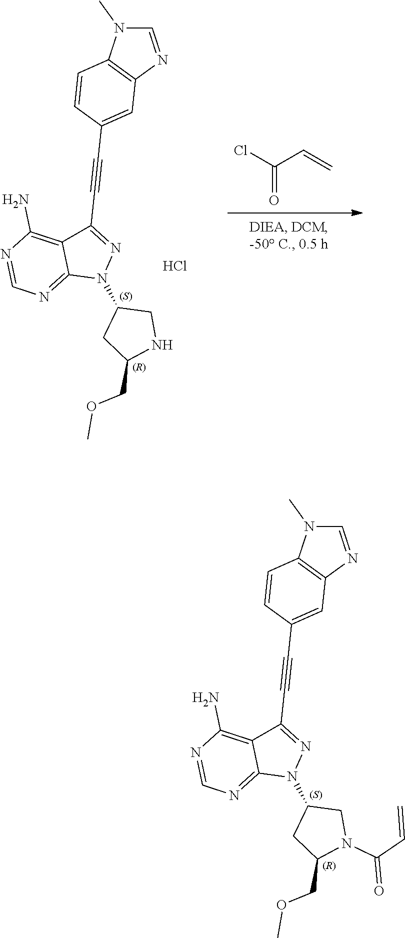

### Step 1: 5-iodo-1-methyl-1H-benzo[d]imidazole & 6-iodo-1-methyl-1H-benzo[d]imidazole

To a mixture of 5-iodo-1H-benzo[d]imidazole (2.4 g, 10.0 mmol) in DMF (20 mL) was added NaH (480 mg, 12.0 mmol) at 0° C. The reaction mixture was stirred at 0° C. for 1 h and then added CH3I (1.7 g, 12.1 mmol). The reaction mixture was stirred at room temperature for 1 h. The reaction mixture was quenched with water (50 mL) and extracted with EA (50 mL) twice. The combined organic layers were washed with brine (50 mL), dried over Na2SO4, filtered and concentrated to give a residue which was purified by silica gel column chromatography (DCM/MeOH=20/1) to afford 5-iodo-1-methyl-1H-benzo[d]imidazole (180 mg, 7%) and 6-iodo-1-methyl-1H-benzo[d]imidazole (200 mg, 8%) as a white solid. [M+H] Calcd.: 259.0; Found, 259.0.

### Step 2: (2R,4S)-tert-butyl 4-(4-amino-3-((1-methyl-1H-benzo[d]imidazol-5-yl)ethynyl)-1H-pyrazolo[3,4-d]pyrimidin-1-yl)-2-(methoxymethyl)pyrrolidine-1-carboxylate

To a solution of (2R,4S)-tert-butyl 4-(4-amino-3-ethynyl-1H-pyrazolo[3,4-d]pyrimidin-1-yl)-2-(methoxymethyl)pyrrolidine-1-carboxylate (235 mg, 0.63 mmol), 5-iodo-1-methyl-1H-benzo[d]imidazole (163 mg, 0.63 mmol) and CuI (19 mg, 0.10 mmol) in DMF (15 mL) was added Pd(PPh3)4 (36 mg, 0.03 mmol), PPh3 (8 mg, 0.03 mmol) and DIEA (244 mg, 1.89 mmol) under nitrogen atmosphere and stirred at 80° C. overnight. The reaction mixture was cooled to rt, diluted with water (30 mL) and extracted with DCM (30 mL) for three times. The combined organic layers were dried over Na2SO4, filtered and concentrated in vacuo to give a residue which was purified by flash (DCM/MeOH=10/1) to afford (2R,4S)-tert-butyl 4-(4-amino-3-((1-methyl-1H-benzo[d]imidazol-5-yl)ethynyl)-1H-pyrazolo[3,4-d]pyrimidin-1-yl)-2-(methoxymethyl)pyrrolidine-1-carboxylate (120 mg, 38%) as a yellow solid. [M+H] Calcd.: 503.2; Found, 503.2.

### Step 3: 1-((3S,5R)-5-(methoxymethyl)pyrrolidin-3-yl)-3-((1-methyl-1H-benzo[d]imidazol-5-yl)ethynyl)-1H-pyrazolo[3,4-d]pyrimidin-4-amine hydrochloride

To a solution of (2R,4S)-tert-butyl 4-(4-amino-3-((1-methyl-1H-benzo[d]imidazol-5-yl)ethynyl)-1H-pyrazolo[3,4-d]pyrimidin-1-yl)-2-(methoxymethyl)pyrrolidine-1-carboxylate (120 mg, 0.24 mmol) in EA (10 mL) was added HCU/EA (10 mL). The reaction mixture was stirred at room temperature for 1 h. The reaction mixture was concentrated in vacuo to afford 1-((3S,5R)-5-(methoxymethyl)pyrrolidin-3-yl)-3-((1-methyl-1H-benzo[d]imidazol-5-yl)ethynyl)-1H-pyrazolo[3,4-d]pyrimidin-4-amine hydrochloride (96 mg, 100%) as a yellow solid. [M+H] Calcd.: 403.2; Found, 403.2.

### Step 4: 1-((2R,4S)-4-(4-amino-3-((1-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of 1-((3S,5R)-5-(methoxymethyl)pyrrolidin-3-yl)-3-((1-methyl-1H-benzo[d]imidazol-5-yl)ethynyl)-1H-pyrazolo[3,4-d]pyrimidin-4-amine hydrochloride (96 mg, 0.24 mmol) and DIEA (93 mg, 0.72 mmol) in DCM (10 mL) at −50° C. under nitrogen atmosphere was added a solution of acryloyl chloride (21 mg, 0.24 mmol) in DCM (0.5 mL) slowly. The mixture was stirred at −50° C. for 0.5 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((1-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (26 mg, 24%) as a yellow solid. ¹H NMR (400 MHz, CDCl₃): 8.38-8.36 (m, 1H), 8.06-7.98 (m, 2H), 7.56-7.44 (m, 2H), 6.61-6.39 (m, 2H), 6.15-6.05 (m, 2H), 5.83-5.68 (m, 2H), 4.66-4.48 (m, 1H), 4.13-4.00 (m, 2H), 3.89 (s, 3H), 3.83-3.81 (m, 1H), 3.54-3.52 (m, 1H), 3.40 (s, 3H), 2.96-2.77 (m, 1H), 2.49-2.47 (m, 1H). [M+H] Calcd.: 457.2; Found, 457.

### Example 7: 1-((2R,4S)-4-(4-amino-3-((1-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

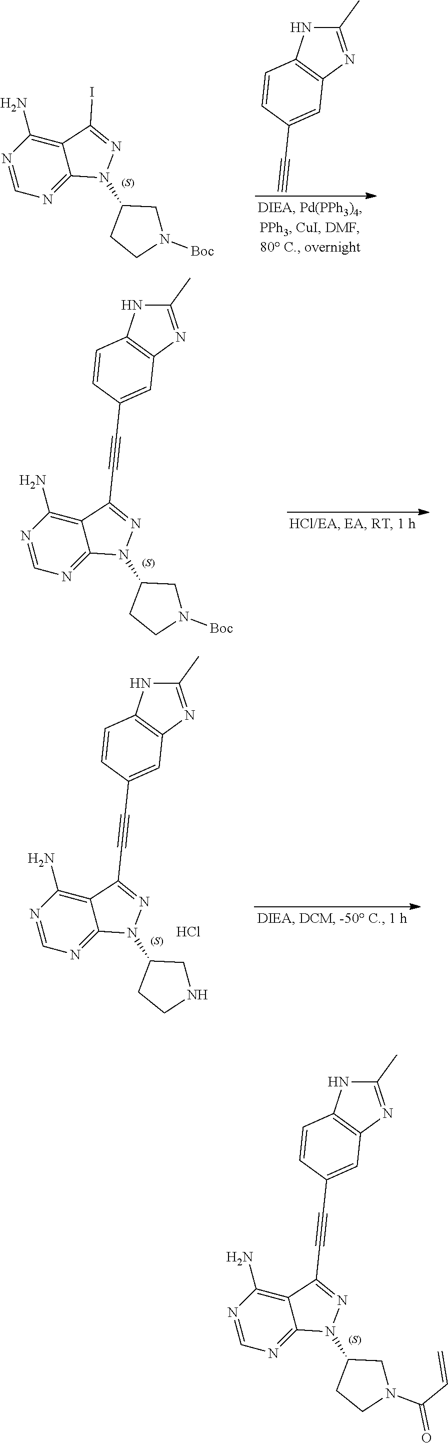

### Step 1: (S)-tert-butyl 3-(4-amino-3-((2-methyl-1H-benzo[d]imidazol-5-yl)ethynyl)-1H-pyrazolo[3,4-d]pyrimidin-1-yl)pyrrolidine-1-carboxylate

To a solution of (S)-tert-butyl 3-(4-amino-3-iodo-1H-pyrazolo[3,4-d]pyrimidin-1-yl)pyrrolidine-1-carboxylate (330 mg, 0.77 mmol), 5-ethynyl-2-methyl-1H-benzo[d]imidazole (100 mg, 0.64 mmol) and CuI (19 mg, 0.10 mmol) in DMF (15 mL) was added Pd(PPh₃)₄ (37 mg, 0.03 mmol), PPh₃ (8 mg, 0.03 mmol) and DIEA (244 mg, 1.89 mmol) under nitrogen atmosphere and stirred at 80° C. overnight. The reaction mixture was cooled to rt, diluted with water (30 mL) and extracted with DCM (30 mL) for three times. The combined organic layers were dried over Na2SO4, filtered and concentrated in vacuo to give a residue which was purified by flash (DCM/MeOH=10/1) to afford (S)-tert-butyl 3-(4-amino-3-((2-methyl-1H-benzo[d]imidazol-5-yl)ethynyl)-1H-pyrazolo[3,4-d]pyrimidin-1-yl)pyrrolidine-1-carboxylate (170 mg, 58%) as a yellow solid. [M+H] Calcd.: 459.2; Found, 459.2.

### Step 2: (S)-3-((2-methyl-1H-benzo[d]imidazol-5-yl)ethynyl)-1-(pyrrolidin-3-yl)-1H-pyrazolo[3,4-d]pyrimidin-4-amine hydrochloride

To a solution of (S)-tert-butyl 3-(4-amino-3-((2-methyl-1H-benzo[d]imidazol-5-yl)ethynyl)-1H-pyrazolo[3,4-d]pyrimidin-1-yl)pyrrolidine-1-carboxylate (170 mg, 0.37 mmol) in EA (10 mL) was added HCl/EA (10 mL). The reaction mixture was stirred at room temperature for 1 h. The reaction mixture was concentrated in vacuo to afford (S)-3-((2-methyl-1H-benzo[d]imidazol-5-yl)ethynyl)-1-(pyrrolidin-3-yl)-1H-pyrazolo[3,4-d]pyrimidin-4-amine hydrochloride (133 mg, 100%) as a white solid. [M+H] Calcd.: 359.2; Found, 359.2.

### Step 3: (S)-1-(3-(4-amino-3-((2-methyl-1H-benzo[d]imidazol-5-yl)ethynyl)-1H-pyrazolo[3,4-d]pyrimidin-1-yl)pyrrolidin-1-yl)prop-2-en-1-one

To a solution of (S)-3-((2-methyl-1H-benzo[d]imidazol-5-yl)ethynyl)-1-(pyrrolidin-3-yl)-1H-pyrazolo[3,4-d]pyrimidin-4-amine hydrochloride (133 mg, 0.37 mmol) and DIEA (143 mg, 1.11 mmol) in DCM (10 mL) at −50° C. under nitrogen atmosphere was added a solution of acryloyl chloride (33 mg, 0.37 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na₂SO₄, filtered and concentrated in vacuo to give a residue which was purified by prep-HPLC to afford (S)-1-(3-(4-amino-3-((2-methyl-1H-benzo[d]imidazol-5-yl)ethynyl)-1H-pyrazolo[3,4-d]pyrimidin-1-yl)pyrrolidin-1-yl)prop-2-en-1-one (31.2 mg, 21%) as a white solid. ¹H NMR (400 MHz, CDCl₃): 8.36 (d, J=5.6 Hz, 1H), 7.77 (s, 1H), 7.52-7.42 (m, 2H), 6.57-6.40 (m, 2H), 5.92 (br s, 2H), 5.75-5.53 (m, 2H), 4.11-3.98 (m, 3H), 3.82-3.75 (m, 1H), 2.66-2.49 (m, 5H). [M+H] Calcd.: 413.2; Found, 413.2.

### Example 8: 1-[(2R,4S)-4-[4-amino-5-[2-(2-methyl-3H-1,3-benzodiazol-5-yl)ethynyl]pyrrolo[2,3-d]pyrimidin-7-yl]-2-(methoxymethyl)pyrrolidin-1-yl]prop-2-en-1-one

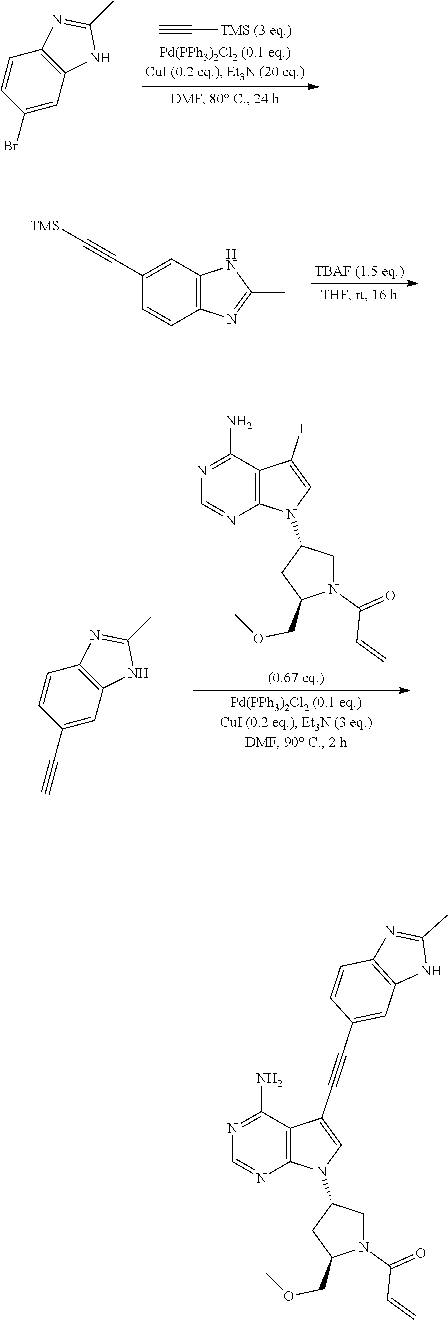

### Step 1: 2-methyl-5-[2-(trimethylsilyl)ethynyl]-3H-1,3-benzodiazole

To a stirred mixture of 5-bromo-2-methyl-3H-1,3-benzodiazole (1.00 g, 4.74 mmol), CuI (0.18 g, 0.95 mmol) and Pd(PPh₃)2Cl₂ (0.33 g, 0.47 mmol) in DMF (20.00 mL) were added trimethylsilylacetylene (4.02 mL, 40.90 mmol) and TEA (13.17 mL, 130.16 mmol) dropwise under nitrogen atmosphere. The reaction mixture was degassed with nitrogen for three times and stirred for 24 h at 80° C. The resulting mixture was diluted with water (100 mL). The resulting mixture was extracted with EtOAc (3×100 mL). The combined organic layers were washed with brine (150 mL), dried over anhydrous Na₂SO₄ and filtered. The filtrate was concentrated under reduced pressure. The residue was purified by silica gel column chromatography, eluted with CH₂Cl₂/MeOH (20:1). The fractions that contained desired product were combined and concentrated to afford 2-methyl-5-[2-(trimethylsilyl)ethynyl]-3H-1,3-benzodiazole (0.32 g, 27%) as a brown solid. MS ESI calculated for C₁₃H₁₆N₂Si [M+H]⁺, 229.11, found 229.15.

### Step 2: 5-ethynyl-2-methyl-3H-1,3-benzodiazole

To a stirred solution of 2-methyl-5-[2-(trimethylsilyl)ethynyl]-3H-1,3-benzodiazole (0.27 g, 1.18 mmol) in THF (2.70 mL, 33.33 mmol) was added 1 M TBAF in THF (1.77 mL, 1.77 mmol) dropwise at 0° C. under nitrogen atmosphere. The resulting mixture was stirred for 16 h at room temperature under nitrogen atmosphere. The resulting mixture was diluted with water (30 mL). The resulting mixture was extracted with EtOAc (3×30 mL). The combined organic layers were washed with brine (50 mL), dried over anhydrous Na₂SO₄ and filtered. The filtrate was concentrated under reduced pressure. The residue was purified by silica gel column chromatography, eluted with DCM/MeOH (20:1). The fractions that contained desired product were combined and concentrated to afford 5-ethynyl-2-methyl-3H-1,3-benzodiazole (0.12 g, 62%) as a light-yellow solid. MS ESI calculated for C₁₀H₈N₂ [M+H]⁺, 157.07, found 157.15.

### Step 3: 1-[(2R,4S)-4-[4-amino-5-[2-(2-methyl-3H-1,3-benzodiazol-5-yl)ethynyl]pyrrolo[2,3-d]pyrimidin-7-yl]-2-(methoxymethyl)pyrrolidin-1-yl]prop-2-en-1-one

To a solution of 1-[(2R,4S)-4-[4-amino-5-iodopyrrolo[2,3-d]pyrimidin-7-yl]-2-(methoxymethyl)pyrrolidin-1-yl]prop-2-en-1-one (0.11 g, 0.26 mmol), 5-ethynyl-2-methyl-3H-1,3-benzodiazole (60.32 mg, 0.39 mmol), CuI (9.81 mg, 0.05 mmol), and TEA (0.11 mL, 1.06 mmol) in DMF (1.00 mL) was added Pd(PPh₃)₂Cl₂ (18.07 mg, 0.03 mmol). The reaction mixture was degassed with nitrogen and stirred for 2 h at 90° C. The resulting mixture was diluted with water (20 mL). The resulting mixture was extracted with EA (3×20 mL). The combined organic layers were washed with brine (50 mL), dried over anhydrous Na₂SO₄ and filtered. The filtrate was concentrated under reduced pressure. The residue was purified by Prep-HPLC with the following conditions Column: Atlantis Prep T3 OBD Column, 19×250 mm 10 μm; Mobile Phase A: Water (0.1% FA), Mobile Phase B: ACN; Flow rate: 20 mL/min; Gradient: 25 B to 50 B in 6 min; 210/254 nm; RT1:5.75. The fractions that contained desired product were combined and concentrated to afford 1-[(2R,4S)-4-[4-amino-5-[2-(2-methyl-3H-1,3-benzodiazol-5-yl)ethynyl]pyrrolo[2,3-d]pyrimidin-7-yl]-2-(methoxymethyl)pyrrolidin-1-yl]prop-2-en-1-one (43.2 mg, 35%) as an off-white solid. MS ESI calculated for C₂₅H₂₅N₇O₂ [M+H]⁺, 456.21, found 456.30. H-NMR (400 MHz, DMSO-d₆): δ 8.17-8.16 (m, 2H), 7.77-7.67 (m, 2H), 7.47 (d, J=8.4 Hz, 1H), 7.30 (d, J=8.0 Hz, 1H), 6.77-6.53 (m, 2H), 6.21-6.15 (m, 1H), 5.73-5.53 (m, 2H), 4.61-4.44 (m, 1H), 4.11-4.07 (m, 1H), 3.85-3.80 (m, 2H), 3.61-3.32 (m, 8H), 2.57-2.50 (m, 1H), 2.36-2.33 (m, 1H).

### Example 9: 1-((2R,4S)-4-(4-amino-3-((1-methyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one

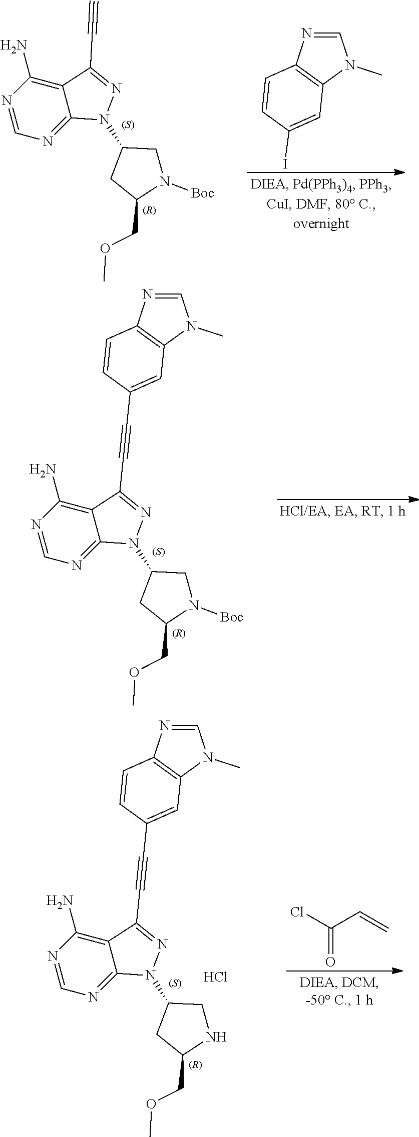

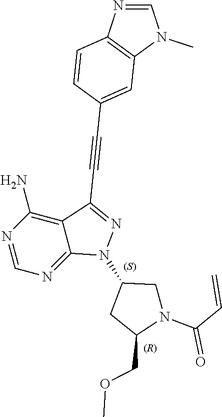

### Step 1: (2R,4S)-tert-butyl 4-(4-amino-3-((1-methyl-1H-benzo[d]imidazol-6-yl)ethynyl)-1H-pyrazolo[3,4-d]pyrimidin-1-yl)-2-(methoxymethyl)pyrrolidine-1-carboxylate

To a solution of (2R,4S)-tert-butyl 4-(4-amino-3-ethynyl-1H-pyrazolo[3,4-d]pyrimidin-1-yl)-2-(methoxymethyl)pyrrolidine-1-carboxylate (235 mg, 0.63 mmol), 6-iodo-1-methyl-1H-benzo[d]imidazole (163 mg, 0.63 mmol) and CuI (19 mg, 0.10 mmol) in DMF (15 mL) was added Pd(PPh₃)₄ (36 mg, 0.03 mmol), PPh₃ (8 mg, 0.03 mmol) and DIEA (244 mg, 1.89 mmol) under nitrogen atmosphere and stirred at 80° C. overnight. The reaction mixture was cooled to rt, diluted with water (30 mL) and extracted with DCM (30 mL) for three times. The combined organic layers were dried over Na₂SO₄, filtered and concentrated in vacuo to give a residue which was purified by flash (DCM/MeOH=10/1) to afford (2R,4S)-tert-butyl 4-(4-amino-3-((1-methyl-1H-benzo[d]imidazol-6-yl)ethynyl)-1H-pyrazolo[3,4-d]pyrimidin-1-yl)-2-(methoxymethyl)pyrrolidine-1-carboxylate (170 mg, 54%) as a yellow solid. [M+H] Calcd.: 503.2; Found, 503.2.

### Step 2: 1-((3S,5R)-5-(methoxymethyl)pyrrolidin-3-yl)-3-((1-methyl-1H-benzo[d]imidazol-6-yl)ethynyl)-1H-pyrazolo[3,4-d]pyrimidin-4-amine hydrochloride

To a solution of (2R,4S)-tert-butyl 4-(4-amino-3-((1-methyl-1H-benzo[d]imidazol-6-yl)ethynyl)-1H-pyrazolo[3,4-d]pyrimidin-1-yl)-2-(methoxymethyl)pyrrolidine-1-carboxylate (170 mg, 0.34 mmol) in EA (10 mL) was added HCl/EA (10 mL). The reaction mixture was stirred at room temperature for 1 h. The reaction mixture was concentrated in vacuo to afford 1-((3S,5R)-5-(methoxymethyl)pyrrolidin-3-yl)-3-((1-methyl-1H-benzo[d]imidazol-6-yl)ethynyl)-1H-pyrazolo[3,4-d]pyrimidin-4-amine hydrochloride (136 mg, 100%) as a yellow solid. [M+H] Calcd.: 403.2; Found, 403.2.

### Step 3: 1-((2R,4S)-4-(4-amino-3-((1-methyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one

To a solution of 1-((3S,5R)-5-(methoxymethyl)pyrrolidin-3-yl)-3-((1-methyl-1H-benzo[d]imidazol-6-yl)ethynyl)-1H-pyrazolo[3,4-d]pyrimidin-4-amine hydrochloride (136 mg, 0.34 mmol) and DIEA (132 mg, 1.02 mmol) in DCM (10 mL) at −50° C. under nitrogen atmosphere was added a solution of acryloyl chloride (30 mg, 0.34 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((1-methyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one (34.7 mg, 22%) as a yellow solid. 1H NMR (400 MHz, CDCl3): 8.39-8.38 (m, 1H), 7.99-7.71 (m, 3H), 7.49 (d, J=7.2 Hz, 1H), 6.62-6.39 (m, 2H), 5.96 (br s, 2H), 5.84-5.67 (m, 2H), 4.66-4.48 (m, 1H), 4.16-4.06 (m, 2H), 3.88 (s, 3H), 3.84-3.82 (m, 1H), 3.55-3.52 (m, 1H), 3.40 (s, 3H), 2.96-2.77 (m, 1H), 2.51-2.48 (m, 1H). [M+H] Calcd.: 457.2; Found, 457.2.

### Example 10: 1-((2R,4S)-4-(3-((1H-benzo[d][1,2,3]triazol-5-yl)ethynyl)-4-amino-1H-pyrazolo[3,4-d]pyrimidin-1-yl)-2-(methoxymethyl)pyrrolidin-1-yl)prop-2-en-1-one

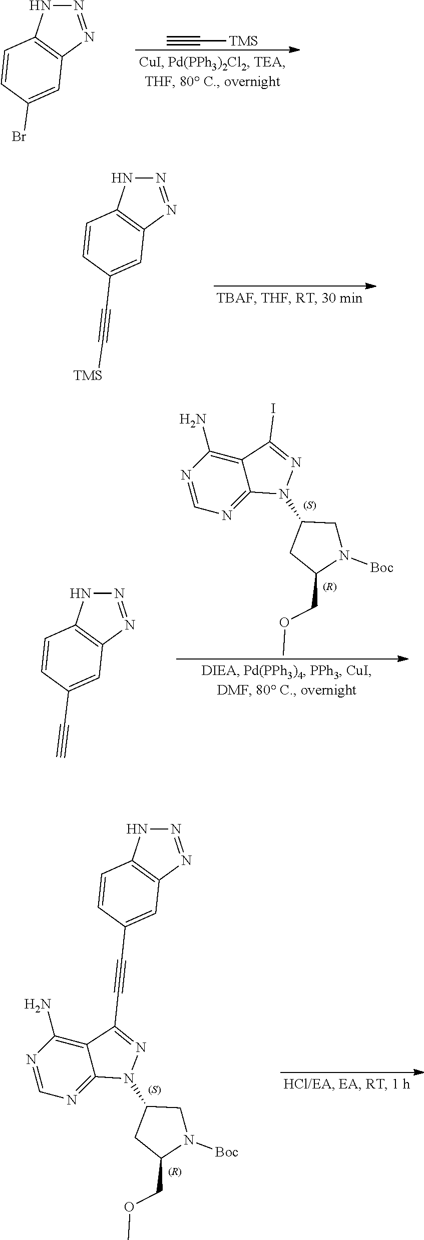

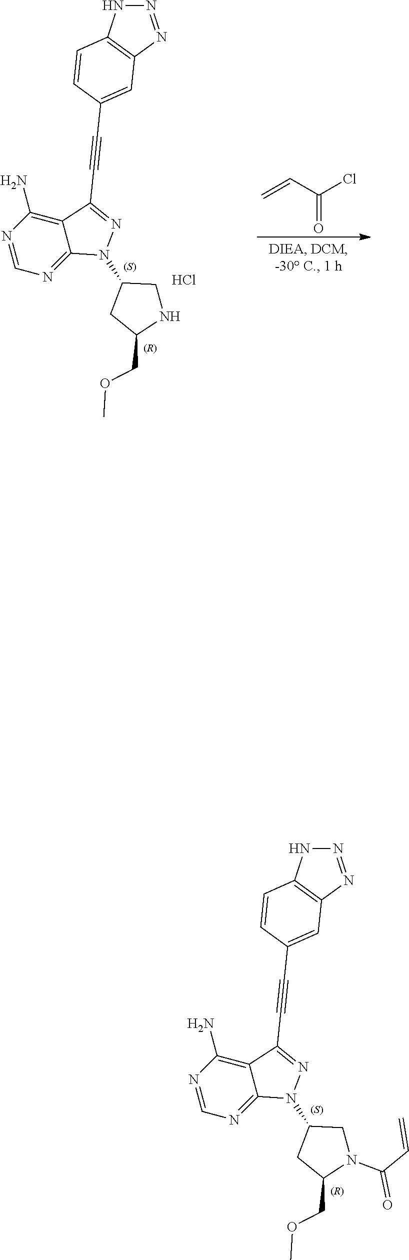

### Step 1: 5-((trimethylsilyl)ethynyl)-1H-benzo[d][1,2,3]triazole

To a solution of 5-bromo-1H-benzo[d][1,2,3]triazole (2.0 g, 10.1 mmol) and CuI (200 mg, 1.05 mmol), Pd(PPh3)2Cl2 (532 mg, 0.76 mmol) in THF (10 mL) and TEA (5 mL) was added ethynyltrimethylsilane (9.8 g, 100.1 mmol). The reaction mixture was stirred at 80° C. overnight under nitrogen atmosphere. The solvent was removed to give a residue which was purified by flash (PE/EA=3/1) to afford 5-((trimethylsilyl)ethynyl)-1H-benzo[d][1,2,3]triazole (800 mg, 38%) as a white solid. [M+H] Calcd.: 216.1; Found, 216.1.

### Step 2: 5-ethynyl-1H-benzo[d][1,2,3]triazole

To a solution of 5-((trimethylsilyl)ethynyl)-1H-benzo[d][1,2,3]triazole (800 mg, 3.72 mmol) in THF (10 mL) was added 1 M TBAF in THF (3.72 mL, 3.72 mmol). The reaction mixture was stirred at room temperature for 30 min. The solvent was removed to give a residue which was purified by flash (PE/EA=1/1) to afford 5-ethynyl-1H-benzo[d][1,2,3]triazole (350 mg, 66%) as a brown solid. [M+H] Calcd.: 144.1; Found, 144.1.

### Step 3: (2R,4S)-tert-butyl 4-(3-((1H-benzo[d][1,2,3]triazol-5-yl)ethynyl)-4-amino-1H-pyrazolo[3,4-d]pyrimidin-1-yl)-2-(methoxymethyl)pyrrolidine-1-carboxylate

To a solution of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.42 mmol), 5-ethynyl-1H-benzo[d][1,2,3]triazole (60 mg, 0.42 mmol) and CuI (11 mg, 0.06 mmol) in DMF (10 mL) was added Pd(PPh3)4 (44 mg, 0.04 mmol), PPh3 (10 mg, 0.04 mmol) and DIEA (248 mg, 1.92 mmol) under nitrogen atmosphere and stirred at 80° C. for 10 h. The reaction mixture was cooled to rt, diluted with water (20 mL) and extracted with DCM (20 mL) for three times. The combined organic layers were dried over Na2SO4, filtered and concentrated in vacuo to give a residue which was purified by flash (DCM/MeOH=10/1) to afford (2R,4S)-tert-butyl 4-(3-((1H-benzo[d][1,2,3]triazol-5-yl)ethynyl)-4-amino-1H-pyrazolo[3,4-d]pyrimidin-1-yl)-2-(methoxymethyl)pyrrolidine-1-carboxylate (100 mg, 48%) as a yellow solid. [M+H] Calcd.: 490.2; Found, 490.2.

### Step 4: 3-((1H-benzo[d][1,2,3]triazol-5-yl)ethynyl)-1-((3S,5R)-5-(methoxymethyl)pyrrolidin-3-yl)-1H-pyrazolo[3,4-d]pyrimidin-4-amine hydrochloride

To a solution of (2R,4S)-tert-butyl 4-(3-((1H-benzo[d][1,2,3]triazol-5-yl)ethynyl)-4-amino-1H-pyrazolo[3,4-d]pyrimidin-1-yl)-2-(methoxymethyl)pyrrolidine-1-carboxylate (100 mg, 0.20 mmol) in EA (10 mL) was added HCl/EA (10 mL). The reaction mixture was stirred at room temperature for 1 h. The reaction mixture was concentrated in vacuo to afford 3-((1H-benzo[d][1,2,3]triazol-5-yl)ethynyl)-1-((3S,5R)-5-(methoxymethyl)pyrrolidin-3-yl)-1H-pyrazolo[3,4-d]pyrimidin-4-amine hydrochloride (90 mg, 100%) as a white solid. [M+H] Calcd.: 390.2; Found, 390.2.

### Step 5: 1-((2R,4S)-4-(3-((1H-benzo[d][1,2,3]triazol-5-yl)ethynyl)-4-amino-1H-pyrazolo[3,4-d]pyrimidin-1-yl)-2-(methoxymethyl)pyrrolidin-1-yl)prop-2-en-1-one

To a solution of 3-((1H-benzo[d][1,2,3]triazol-5-yl)ethynyl)-1-((3S,5R)-5-(methoxymethyl)pyrrolidin-3-yl)-1H-pyrazolo[3,4-d]pyrimidin-4-amine hydrochloride (80 mg, 0.21 mmol) and DIEA (52 mg, 0.40 mmol) in DCM (10 mL) at −30° C. under nitrogen atmosphere was added a solution of acryloyl chloride (18 mg, 0.21 mmol) in DCM (0.5 mL) slowly. The mixture was stirred at −30° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(3-((1H-benzo[d][1,2,3]triazol-5-yl)ethynyl)-4-amino-1H-pyrazolo[3,4-d]pyrimidin-1-yl)-2-(methoxymethyl)pyrrolidin-1-yl)prop-2-en-1-one (64.8 mg, 44%) as a white solid. 1H NMR (400 MHz, CDCl3): 8.39-8.35 (m, 1H), 8.16 (br s, 1H), 7.78-7.72 (m, 1H), 7.51-7.42 (m, 1H), 6.62-6.42 (m, 2H), 5.79-5.64 (m, 2H), 4.70-4.55 (m, 1H), 4.27-3.84 (m, 3H), 3.54-3.52 (m, 2H), 3.48 (s, 3H), 2.90-2.70 (m, 1H), 2.53-2.49 (m, 1H). [M+H] Calcd.: 444.2; Found, 444.2.

### Example 11: (S)-1-(3-(4-amino-5-((2-methyl-1H-benzo[d]imidazol-5-yl)ethynyl)-7H-pyrrolo[2,3-d]pyrimidin-7-yl)pyrrolidin-1-yl)prop-2-en-1-one

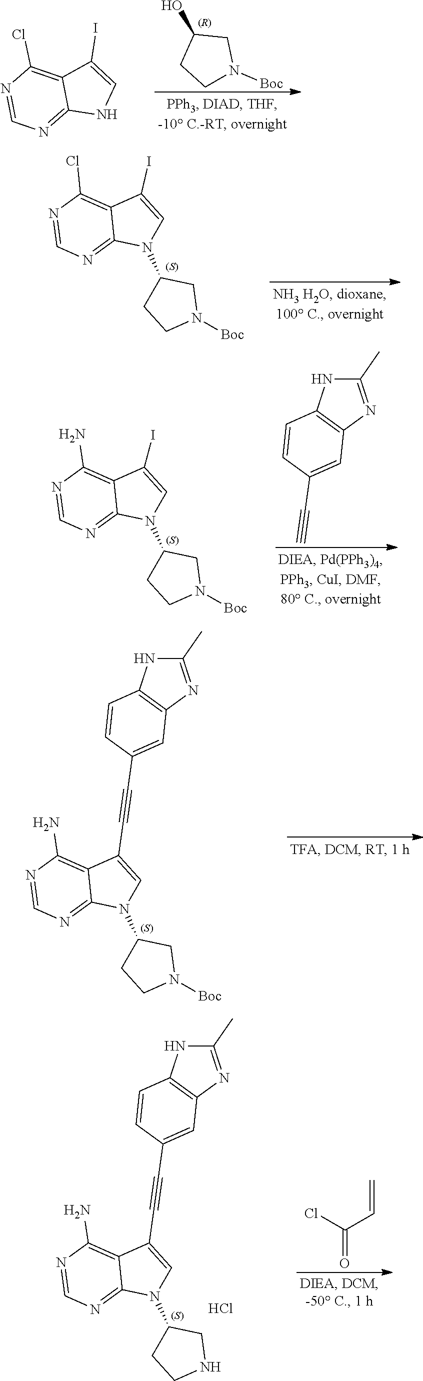

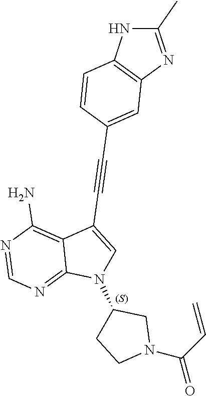

### Step 1: (S)-tert-butyl 3-(4-chloro-5-iodo-7H-pyrrolo[2,3-d]pyrimidin-7-yl)pyrrolidine-1-carboxylate

To a stirred solution of 4-chloro-5-iodo-7H-pyrrolo[2,3-d]pyrimidine (10.1 g, 36.2 mmol), (R)-tert-butyl3-hydroxypyrrolidine-1-carboxylate (13.6 g, 72.4 mmol) and PPh3 (17.1 g, 65.1 mmol) in anhydrous THF (200 mL) was slowly added DIAD (10.9 g, 54.2 mmol) over 1 h at −10° C. and under nitrogen atmosphere. The resulting reaction mixture was subsequently warmed up to room temperature and stirred overnight. The solvent was evaporated and the residue was purified by a flash (PE/EA=5/1) to give (S)-tert-butyl 3-(4-chloro-5-iodo-7H-pyrrolo[2,3-d]pyrimidin-7-yl)pyrrolidine-1-carboxylate (9.9 g, 60%) as a white solid. [M+H] Calcd.: 449.0, found: 449.0.

### Step 2: (S)-tert-butyl 3-(4-amino-5-iodo-7H-pyrrolo[2,3-d]pyrimidin-7-yl)pyrrolidine-1-carboxylate

A solution of (S)-tert-butyl 3-(4-chloro-5-iodo-7H-pyrrolo[2,3-d]pyrimidin-7-yl)pyrrolidine-1-carboxylate (5.0 g, 11.2 mmol) and NH40H (20 mL) in 1.4-dioxane (10 mL) was stirred in an autoclave at 100° C. overnight. The mixture was allowed to cool to room temperature and concentrated in vacuo. The reaction mixture was added water (30 mL). The resulting solid was collected by filtration and dried in vacuo to afford (S)-tert-butyl 3-(4-amino-5-iodo-7H-pyrrolo[2,3-d]pyrimidin-7-yl)pyrrolidine-1-carboxylate (3.5 g, 73%) as white solid. [M+H] Calcd.: 430.1, found: 430.1.

### Step 3: (S)-tert-butyl 3-(4-amino-5-((2-methyl-1H-benzo[d]imidazol-5-yl)ethynyl)-7H-pyrrolo[2,3-d]pyrimidin-7-yl)pyrrolidine-1-carboxylate

To a solution of (S)-tert-butyl 3-(4-amino-5-iodo-7H-pyrrolo[2,3-d]pyrimidin-7-yl)pyrrolidine-1-carboxylate (130 mg, 0.30 mmol), 5-ethynyl-2-methyl-1H-benzo[d]imidazole (57 mg, 0.30 mmol) and CuI (11 mg, 0.06 mmol) in DMF (10 mL) was added Pd(PPh3)4 (17 mg, 0.02 mmol), PPh3 (5 mg, 0.02 mmol) and DIEA (116 mg, 0.90 mmol) under nitrogen atmosphere and stirred at 80° C. overnight. The reaction mixture was cooled to rt, diluted with water (20 mL) and extracted with DCM (20 mL) for three times. The combined organic layers were dried over Na2SO4, filtered and concentrated in vacuo to give a residue which was purified by flash (DCM/MeOH=10/1) to afford (S)-tert-butyl 3-(4-amino-5-((2-methyl-1H-benzo[d]imidazol-5-yl)ethynyl)-7H-pyrrolo[2,3-d]pyrimidin-7-yl)pyrrolidine-1-carboxylate (40 mg, 29%) as a yellow solid. [M+H] Calcd.: 458.2; Found, 458.2.

### Step 4: TFA salt of (S)-5-((2-methyl-1H-benzo[d]imidazol-5-yl)ethynyl)-7-(pyrrolidin-3-yl)-7H-pyrrolo[2,3-d]pyrimidin-4-amine

To a solution of (S)-tert-butyl 3-(4-amino-5-((2-methyl-1H-benzo[d]imidazol-5-yl)ethynyl)-7H-pyrrolo[2,3-d]pyrimidin-7-yl)pyrrolidine-1-carboxylate (40 mg, 0.09 mmol) in DCM (10 mL) was added TFA (1 mL). The reaction mixture was stirred at room temperature for 1 h. The reaction mixture was concentrated in vacuo to afford TFA salt of (S)-5-((2-methyl-1H-benzo[d]imidazol-5-yl)ethynyl)-7-(pyrrolidin-3-yl)-7H-pyrrolo[2,3-d]pyrimidin-4-amine (31 mg, 100%) as yellow oil. [M+H] Calcd.: 358.2; Found, 358.2.

### Step 5: (S)-1-(3-(4-amino-5-((2-methyl-1H-benzo[d]imidazol-5-yl)ethynyl)-7H-pyrrolo[2,3-d]pyrimidin-7-yl)pyrrolidin-1-yl)prop-2-en-1-one

To a solution of TFA salt of (S)-5-((2-methyl-1H-benzo[d]imidazol-5-yl)ethynyl)-7-(pyrrolidin-3-yl)-7H-pyrrolo[2,3-d]pyrimidin-4-amine (31 mg, 0.09 mmol) and DIEA (35 mg, 0.27 mmol) in DCM (10 mL) at −50° C. under nitrogen atmosphere was added a solution of acryloyl chloride (8 mg, 0.09 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford (S)-1-(3-(4-amino-5-((2-methyl-1H-benzo[d]imidazol-5-yl)ethynyl)-7H-pyrrolo[2,3-d]pyrimidin-7-yl)pyrrolidin-1-yl)prop-2-en-1-one (7.3 mg, 20%) as a white solid. 1H NMR (400 MHz, CDCl3): 8.30 (s, 1H), 7.69 (s, 1H), 7.54-7.52 (m, 1H), 7.37 (d, J=8.4 Hz, 1H), 7.21 (d, J=10.4 Hz, 1H), 6.51-6.41 (m, 2H), 5.94-5.90 (m, 2H), 5.80-5.72 (m, 1H), 5.50-5.43 (m, 1H), 4.18-3.79 (m, 4H), 2.68 (s, 3H), 2.57-2.36 (m, 2H). [M+H] Calcd.: 412.2; Found, 412.2.

### Example 12: 1-((2R,4S)-4-(4-amino-3-((4,6-difluoro-1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

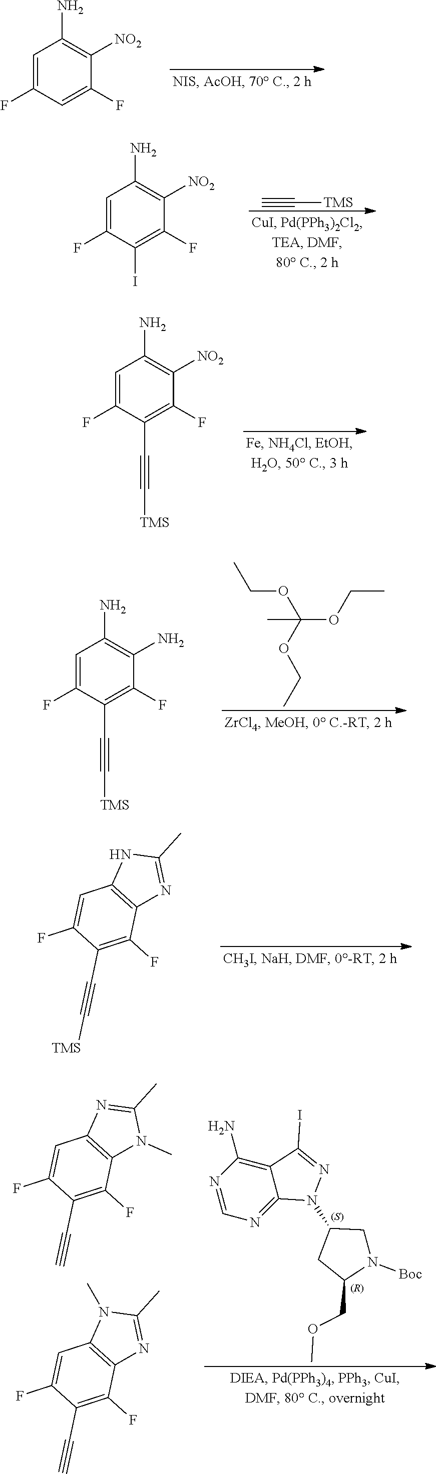

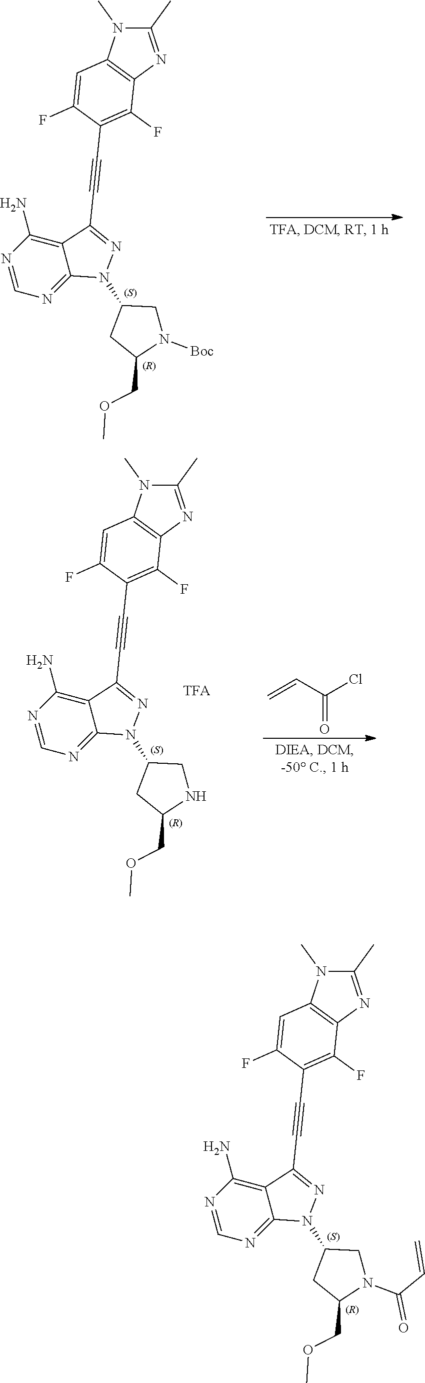

### Step 1: 3,5-difluoro-4-iodo-2-nitroaniline

A mixture of 3,5-difluoro-2-nitroaniline (800 mg, 4.60 mmol) and NIS (1.1 g, 5.10 mmol) in AcOH (10 mL) was stirred at 70° C. for 3 h. Then AcOH was removed, added saturated NaHCO₃ aqueous solution (50 mL) and extracted with EA (50 mL) for three times. The combined organic layers were dried over Na2SO4, filtered and concentrated in vacuo to give a residue which was purified by flash (PE/EA=5/1) to afford 3,5-difluoro-4-iodo-2-nitroaniline (1.0 g, 65%) as a yellow solid.

### Step 2: 3,5-difluoro-2-nitro-4-((trimethylsilyl)ethynyl)aniline

To a solution of 3,5-difluoro-4-iodo-2-nitroaniline (800 mg, 2.67 mmol) and CuI (51 mg, 0.27 mmol), Pd(PPh3)2Cl2 (94 mg, 0.13 mmol) in DMF (20 mL) was added ethynyltrimethylsilane (523 mg, 5.34 mmol) and TEA (404 mg, 4.00 mmol). The reaction mixture was stirred at 80° C. for 2 h under nitrogen atmosphere. The solvent was removed to give a residue which was purified by flash (PE/EA=5/1) to afford 3,5-difluoro-2-nitro-4-((trimethylsilyl)ethynyl)aniline (650 mg, 90%) as a yellow solid. [M+H] Calcd.: 271.1; Found, 271.1.

### Step 3: 3,5-difluoro-4-((trimethylsilyl)ethynyl)benzene-1,2-diamine

To a solution of 3,5-difluoro-2-nitro-4-((trimethylsilyl)ethynyl)aniline (650 mg, 2.41 mmol) and NH4Cl (1.3 g, 24.1 mmol) in EtOH (20 mL) and H2O (10 mL) was added iron (1.3 g, 24.1 mmol) at 50° C. The mixture was stirred at 50° C. for 3 h. The reaction mixture was filtered and concentrated. The residue was purified by flash (PE/EA=1/1) to give 3,5-difluoro-4-((trimethylsilyl)ethynyl)benzene-1,2-diamine (270 mg, 47%) as yellow oil. [M+H] Calcd.: 241.1; Found, 241.1.

### Step 4: 4,6-difluoro-2-methyl-5-((trimethylsilyl)ethynyl)-1H-benzo[d]imidazole

To a solution of 3,5-difluoro-4-((trimethylsilyl)ethynyl)benzene-1,2-diamine (270 mg, 1.13 mmol) in MeOH (20 mL) was added 1,1,1-triethoxyethane (219 mg, 1.35 mmol) and ZrCl4 (26 mg, 0.11 mmol) at 0° C. The mixture was stirred at room temperature for 2 h. The reaction mixture was filtered and concentrated. The residue was purified by flash (PE/EA=1/1) to give 4,6-difluoro-2-methyl-5-((trimethylsilyl)ethynyl)-1H-benzo[d]imidazole (160 mg, 54%) as a yellow solid. [M+H] Calcd.: 265.1; Found, 265.1.

### Step 5: 5-ethynyl-4,6-difluoro-1,2-dimethyl-1H-benzo[d]imidazole & 6-ethynyl-5,7-difluoro-1,2-dimethyl-1H-benzo[d]imidazole

To a solution of 4,6-difluoro-2-methyl-5-((trimethylsilyl)ethynyl)-1H-benzo[d]imidazole (160 mg, 0.61 mmol) in DMF (10 mL) was added NaH (29 mg, 0.73 mmol) at 0° C. The mixture was stirred at 0° C. for 30 min and then added dropwise CH3I (103 mg, 0.73 mmol). The mixture was stirred at room temperature for 2 h. The mixture was cooled to rt, diluted with water (10 mL) and extracted with EA (10 mL) for three times. The combined organic layers were dried over Na2SO4, filtered and concentrated in vacuo to give a residue which was purified by flash (100% EA) to afford 5-ethynyl-4,6-difluoro-1,2-dimethyl-1H-benzo[d]imidazole (50 mg, 40%) and 6-ethynyl-5,7-difluoro-1,2-dimethyl-1H-benzo[d]imidazole (30 mg, 24%) as a yellow solid. [M+H] Calcd.: 207.1; Found, 207.1.

### Step 6: (2R,4S)-tert-butyl 4-(4-amino-3-((4,6-difluoro-1,2-dimethyl-1H-benzo[d]imidazol-5-yl)ethynyl)-1H-pyrazolo[3,4-d]pyrimidin-1-yl)-2-(methoxymethyl)pyrrolidine-1-carboxylate

To a solution of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.42 mmol), 5-ethynyl-4,6-difluoro-1,2-dimethyl-1H-benzo[d]imidazole (60 mg, 0.29 mmol) and CuI (9 mg, 0.05 mmol) in DMF (10 mL) was added Pd(PPh3)4 (17 mg, 0.02 mmol), PPh3 (5 mg, 0.02 mmol) and DIEA (116 mg, 0.92 mmol) under nitrogen atmosphere and stirred at 80° C. overnight. The reaction mixture was cooled to rt, diluted with water (20 mL) and extracted with DCM (20 mL) for three times. The combined organic layers were dried over Na2SO4, filtered and concentrated in vacuo to give a residue which was purified by flash (DCM/MeOH=10/1) to afford (2R,4S)-tert-butyl 4-(4-amino-3-((4,6-difluoro-1,2-dimethyl-1H-benzo[d]imidazol-5-yl)ethynyl)-1H-pyrazolo[3,4-d]pyrimidin-1-yl)-2-(methoxymethyl)pyrrolidine-1-carboxylate (50 mg, 30%) as a yellow solid. [M+H] Calcd.: 553.2; Found, 553.2.

### Step 7: TFA salt of 3-((4,6-difluoro-1,2-dimethyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine

To a solution of (2R,4S)-tert-butyl 4-(4-amino-3-((4,6-difluoro-1,2-dimethyl-1H-benzo[d]imidazol-5-yl)ethynyl)-1H-pyrazolo[3,4-d]pyrimidin-1-yl)-2-(methoxymethyl)pyrrolidine-1-carboxylate (50 mg, 0.09 mmol) in DCM (10 mL) was added TFA (3 mL). The reaction mixture was stirred at room temperature for 1 h. The reaction mixture was concentrated in vacuo to afford a TFA salt of 3-((4,6-difluoro-1,2-dimethyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine (41 mg, 100%) as yellow oil. [M+H] Calcd.: 453.2; Found, 453.2.

### Step 8: 1-((2R,4S)-4-(4-amino-3-((4,6-difluoro-1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of TFA salt of 3-((4,6-difluoro-1,2-dimethyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine (41 mg, 0.09 mmol) and DIEA (35 mg, 0.27 mmol) in DCM (10 mL) at −50° C. under nitrogen atmosphere was added a solution of acryloyl chloride (8 mg, 0.09 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((4,6-difluoro-1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (19.2 mg, 42%) as a white solid. 1H NMR (400 MHz, CDCl3): 8.42 (br s, 1H), 6.93-6.55 (m, 1H), 6.44-6.40 (m, 2H), 6.28 (br s, 2H), 5.85-5.67 (m, 2H), 4.66-4.50 (m, 1H), 4.20-4.14 (m, 2H), 4.03 (d, J=12.0 Hz, 1H), 3.73 (s, 3H), 3.54-3.51 (m, 1H), 3.40 (s, 3H), 2.95-2.76 (m, 1H), 2.62-2.45 (m, 4H). [M+H] Calcd.: 507.2; Found, 507.2.

### Example 13: 1-((2R,4S)-4-(4-amino-3-((5,7-difluoro-1,2-dimethyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one

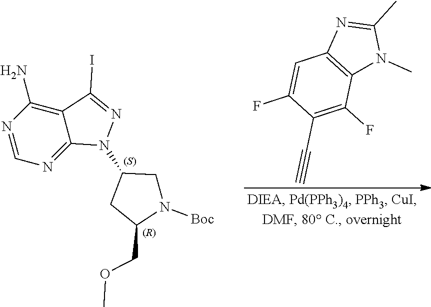

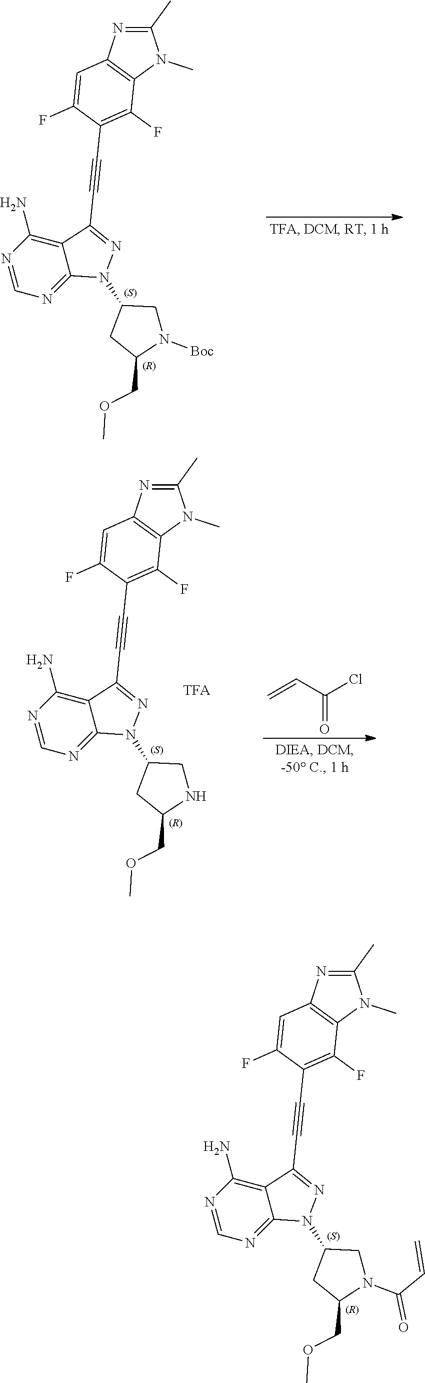

### Step 1: (2R,4S)-tert-butyl 4-(4-amino-3-((5,7-difluoro-1,2-dimethyl-1H-benzo[d]imidazol-6-yl)ethynyl)-1H-pyrazolo[3,4-d]pyrimidin-1-yl)-2-(methoxymethyl)pyrrolidine-1-carboxylate

To a solution of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.42 mmol), 6-ethynyl-5,7-difluoro-1,2-dimethyl-1H-benzo[d]imidazole (60 mg, 0.29 mmol) and CuI (9 mg, 0.05 mmol) in DMF (10 mL) was added Pd(PPh3)4 (17 mg, 0.02 mmol), PPh3 (5 mg, 0.02 mmol) and DIEA (116 mg, 0.92 mmol) under nitrogen atmosphere and stirred at 80° C. overnight. The reaction mixture was cooled to rt, diluted with water (20 mL) and extracted with DCM (20 mL) for three times. The combined organic layers were dried over Na2SO4, filtered and concentrated in vacuo to give a residue which was purified by flash (DCM/MeOH=10/1) to afford (2R,4S)-tert-butyl 4-(4-amino-3-((5,7-difluoro-1,2-dimethyl-1H-benzo[d]imidazol-6-yl)ethynyl)-1H-pyrazolo[3,4-d]pyrimidin-1-yl)-2-(methoxymethyl)pyrrolidine-1-carboxylate (30 mg, 18%) as a yellow solid. [M+H] Calcd.: 553.2; Found, 553.2.

### Step 2: TFA salt of 3-((5,7-difluoro-1,2-dimethyl-1H-benzo[d]imidazol-6-yl)ethynyl)-1-((3S,5R)-5-(methoxymethyl)pyrrolidin-3-yl)-1H-pyrazolo[3,4-d]pyrimidin-4-amine

To a solution of (2R,4S)-tert-butyl 4-(4-amino-3-((5,7-difluoro-1,2-dimethyl-1H-benzo[d]imidazol-6-yl)ethynyl)-1H-pyrazolo[3,4-d]pyrimidin-1-yl)-2-(methoxymethyl)pyrrolidine-1-carboxylate (30 mg, 0.05 mmol) in DCM (10 mL) was added TFA (3 mL). The reaction mixture was stirred at room temperature for 1 h. The reaction mixture was concentrated in vacuo to afford a TFA salt of 3-((5,7-difluoro-1,2-dimethyl-1H-benzo[d]imidazol-6-yl)ethynyl)-1-((3S,5R)-5-(methoxymethyl)pyrrolidin-3-yl)-1H-pyrazolo[3,4-d]pyrimidin-4-amine (25 mg, 100%) as yellow oil. [M+H] Calcd.: 453.2; Found, 453.2.

### Step 3: 1-((2R,4S)-4-(4-amino-3-((5,7-difluoro-1,2-dimethyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one

To a solution of TFA salt of 3-((5,7-difluoro-1,2-dimethyl-1H-benzo[d]imidazol-6-yl)ethynyl)-1-((3S,5R)-5-(methoxymethyl)pyrrolidin-3-yl)-1H-pyrazolo[3,4-d]pyrimidin-4-amine (25 mg, 0.06 mmol) and DIEA (23 mg, 0.18 mmol) in DCM (10 mL) at −50° C. under nitrogen atmosphere was added a solution of acryloyl chloride (5 mg, 0.06 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((5,7-difluoro-1,2-dimethyl-1H-benzo[d]imidazol-6-yl)ethynyl)-1H-pyrazolo[3,4-d]pyrimidin-1-yl)-2-(methoxymethyl)pyrrolidin-1-yl)prop-2-en-1-one (7.6 mg, 27%) as a white solid. 1H NMR (400 MHz, CDCl3): 8.39 (s, 1H), 7.22 (s, 1H), 6.57-6.41 (m, 2H), 5.83-5.68 (m, 3H), 4.67-4.48 (m, 1H), 4.13-4.05 (m, 2H), 3.94 (s, 3H), 3.80-3.77 (m, 1H), 3.54-3.51 (m, 1H), 3.40 (s, 3H), 2.93-2.76 (m, 1H), 2.61 (s, 3H), 2.50-2.45 (m, 1H). [M+H] Calcd.: 507.2; Found, 507.2.

### Example 14: 1-((2R,4S)-4-(3-((1H-indazol-6-yl)ethynyl)-4-amino-1H-pyrazolo[3,4-d]pyrimidin-1-yl)-2-(methoxymethyl)pyrrolidin-1-yl)prop-2-en-1-one

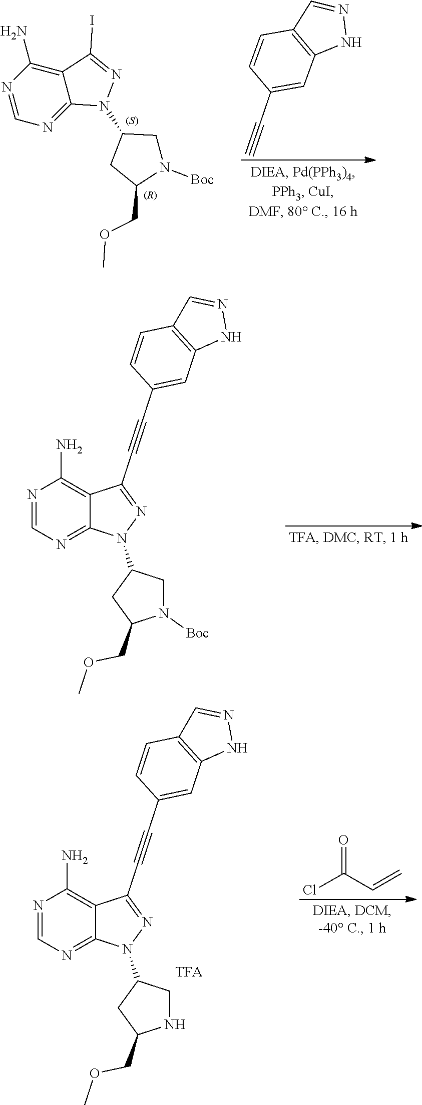

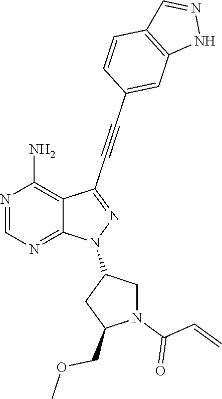

### Step 1: (2R,4S)-tert-butyl 4-(3-((1H-indazol-6-yl)ethynyl)-4-amino-1H-pyrazolo[3,4-d]pyrimidin-1-yl)-2-(methoxymethyl)pyrrolidine-1-carboxylate

To a solution of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.42 mmol), 6-ethynyl-1H-indazole (71 mg, 0.51 mmol) and CuI (2 mg, 0.01 mmol) in DMF (10 mL) was added Pd(PPh3)4 (243 mg, 0.21 mmol), PPh3 (6 mg, 0.02 mmol) and DIEA (163 mg, 1.26 mmol) under nitrogen atmosphere and stirred at 80° C. for 16 h. The reaction mixture was cooled to rt, diluted with water (30 mL) and extracted with DCM (20 mL) for three times. The combined organic layers were dried over Na2SO4, filtered and concentrated in vacuo to give a residue which was purified by flash (DCM/MeOH=10/1) to afford (2R,4S)-tert-butyl 4-(3-((1H-indazol-6-yl)ethynyl)-4-amino-1H-pyrazolo[3,4-d]pyrimidin-1-yl)-2-(methoxymethyl)pyrrolidine-1-carboxylate (150 mg, 75%) as a white solid. [M+H] Calcd.: 489.2; Found, 489.2.

### Step 2: TFA salt of 3-((1H-indazol-6-yl)ethynyl)-1-((3S,5R)-5-(methoxymethyl)pyrrolidin-3-yl)-1H-pyrazolo[3,4-d]pyrimidin-4-amine

To a solution of (2R,4S)-tert-butyl 4-(3-((1H-indazol-6-yl)ethynyl)-4-amino-1H-pyrazolo[3,4-d]pyrimidin-1-yl)-2-(methoxymethyl)pyrrolidine-1-carboxylate (150 mg, 0.31 mmol) in DCM (10 mL) was added TFA (0.5 mL). The reaction mixture was stirred at room temperature for 1 h. The reaction mixture was concentrated in vacuo to afford TFA salt of 3-((1H-indazol-6-yl)ethynyl)-1-((3S,5R)-5-(methoxymethyl)pyrrolidin-3-yl)-1H-pyrazolo[3,4-d]pyrimidin-4-amine (119 mg, 100%) as yellow oil. [M+H] Calcd.: 389.2; Found, 389.2.

### Step 3: 1-((2R,4S)-4-(3-((1H-indazol-6-yl)ethynyl)-4-amino-1H-pyrazolo[3,4-d]pyrimidin-1-yl)-2-(methoxymethyl)pyrrolidin-1-yl)prop-2-en-1-one

To a solution of TFA salt of 3-((1H-indazol-6-yl)ethynyl)-1-((3S,5R)-5-(methoxymethyl)pyrrolidin-3-yl)-1H-pyrazolo[3,4-d]pyrimidin-4-amine (119 mg, 0.31 mmol) and DIEA (118 mg, 0.92 mmol) in DCM (15 mL) at −40° C. under nitrogen atmosphere was added a solution of acryloyl chloride (25 mg, 0.28 mmol) in DCM (0.5 mL) slowly. The mixture was stirred at −40° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(3-((1H-indazol-6-yl)ethynyl)-4-amino-1H-pyrazolo[3,4-d]pyrimidin-1-yl)-2-(methoxymethyl)pyrrolidin-1-yl)prop-2-en-1-one (21.7 mg, 16%) as a white solid. 1H NMR (400 MHz, DMSO-d6): 13.32 (s, 1H), 8.27 (d, J=2.8 Hz, 1H), 8.15 (s, 1H), 7.97 (s, 1H), 7.84 (d, J=8.4 Hz, 1H), 7.40-6.52 (m, 4H), 6.19-6.18 (m, 1H), 5.86-5.65 (m, 2H), 4.61-4.47 (m, 1H), 4.05-3.96 (m, 2H), 3.82-3.47 (m, 3H), 3.31-3.29 (m, 2H), 2.68-2.50 (m, 1H), 2.40-2.37 (m, 1H). [M+H] Calcd.: 443.2; Found, 443.2.

### Example 15: 1-((2R,4S)-4-(4-amino-3-((1-cyclopropyl-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

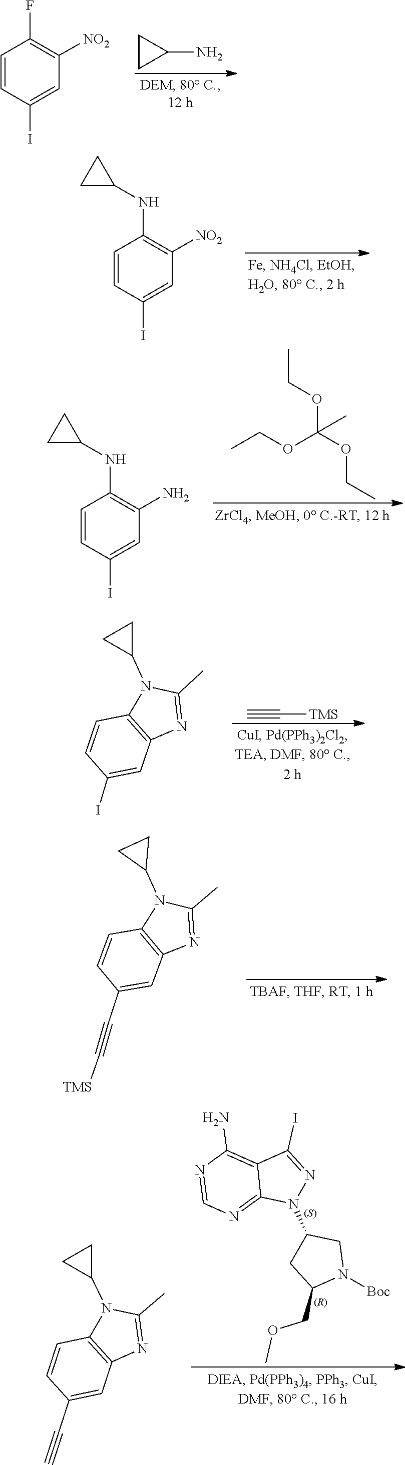

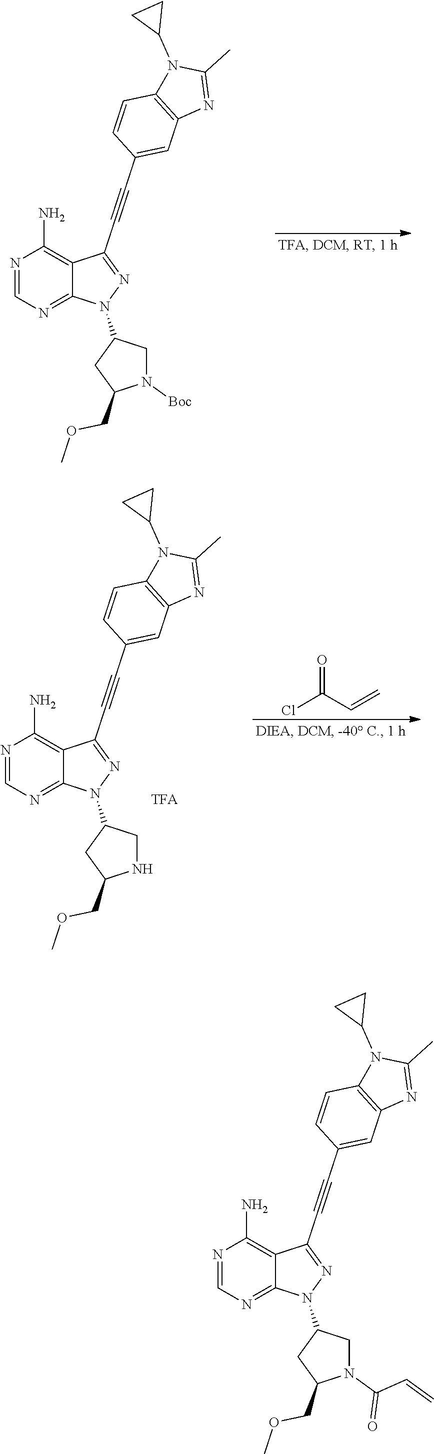

### Step 1: N-cyclopropyl-4-iodo-2-nitroaniline

A mixture of 1-fluoro-4-iodo-2-nitrobenzene (3.0 g, 11.2 mmol) and cyclopropanamine (1.3 g, 22.4 mmol) in DEM (20 mL) was stirred at 80° C. for 12 h. The reaction mixture was quenched with water (50 mL) and extracted with DCM (50 mL) twice. The combined organic layers were dried over Na2SO4, filtered and concentrated in vacuo to give a crude N-cyclopropyl-4-iodo-2-nitroaniline (3.0 g, 98%) as a white solid. [M+H] Calcd.: 305.0; Found, 305.0.

### Step 2: N1-cyclopropyl-4-iodobenzene-1,2-diamine

To a solution of N-cyclopropyl-4-iodo-2-nitroaniline (3.0 g, 10.0 mmol) and NH4C1 (2.2 g, 40.0 mmol) in EtOH (30 mL) and H2O (6 mL) was added iron (2.2 g, 40.0 mmol). The reaction mixture was stirred at 80° C. for 2 h. The reaction mixture was cooled to rt, filtered and concentrated. The residue was purified by flash (PE/EA=3/1) to give N1-cyclopropyl-4-iodobenzene-1,2-diamine (1.8 g, 67%) as a brown oil. [M+H] Calcd.: 275.0; Found, 275.0.

### Step 3: 1-cyclopropyl-5-iodo-2-methyl-1H-benzo[d]imidazole

To a solution of N1-cyclopropyl-4-iodobenzene-1,2-diamine (3.0 g, 10.9 mmol) in MeOH (50 mL) was added 1,1,1-triethoxyethane (3.5 g, 22.0 mmol) and ZrCl4 (233 mg, 1.09 mmol) at 0° C. The mixture was stirred at room temperature for 12 h. The reaction mixture was filtered and concentrated. The residue was purified by flash (PE/EA=3/1) to give 1-cyclopropyl-5-iodo-2-methyl-1H-benzo[d]imidazole (1.5 g, 50%) as a yellow solid. [M+H] Calcd.: 299.0; Found, 299.0.

### Step 4: 1-cyclopropyl-2-methyl-5-((trimethylsilyl)ethynyl)-1H-benzo[d]imidazole

To a solution of 1-cyclopropyl-5-iodo-2-methyl-1H-benzo[d]imidazole (1.2 g, 4.0 mmol) and CuI (76 mg, 0.40 mmol), Pd(PPh3)2Cl2 (141 mg, 0.20 mmol) in DMF (30 mL) was added ethynyltrimethylsilane (789 mg, 8.05 mmol) and TEA (610 mg, 6.04 mmol). The reaction mixture was stirred at 80° C. for 2 h under nitrogen atmosphere. The solvent was removed to give a residue which was purified by flash (PE/EA=1/1) to afford 1-cyclopropyl-2-methyl-5-((trimethylsilyl)ethynyl)-1H-benzo[d]imidazole (800 mg, 80%) as a white solid. [M+H] Calcd.: 269.1; Found, 269.1.

### Step 5: 1-cyclopropyl-5-ethynyl-2-methyl-1H-benzo[d]imidazole

To a mixture of 1-cyclopropyl-2-methyl-5-((trimethylsilyl)ethynyl)-1H-benzo[d]imidazole (800 mg, 2.97 mmol) in THF (20 mL) was added 1 M TBAF in THF (3.6 mL, 3.6 mmol). The reaction mixture was stirred at room temperature for 1 h. The reaction mixture was quenched with water (30 mL) and extracted with EA (30 mL) twice. The combined organic layers were washed with brine (30 mL), dried over Na2SO4, filtered and concentrated to give a residue which was purified by silica gel column chromatography (PE/EA=1/1) to afford 1-cyclopropyl-5-ethynyl-2-methyl-1H-benzo[d]imidazole (420 mg, 72%) as a white solid. [M+H] Calcd.: 197.1; Found, 197.1.

### Step 6: (2R,4S)-tert-butyl 4-(4-amino-3-((1-cyclopropyl-2-methyl-1H-benzo[d]imidazol-5-yl)ethynyl)-1H-pyrazolo[3,4-d]pyrimidin-1-yl)-2-(methoxymethyl)pyrrolidine-1-carboxylate

To a solution of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.42 mmol), 1-cyclopropyl-5-ethynyl-2-methyl-1H-benzo[d]imidazole (99 mg, 0.51 mmol) and CuI (2 mg, 0.01 mmol) in DMF (10 mL) was added Pd(PPh3)4 (243 mg, 0.21 mmol), PPh3 (6 mg, 0.02 mmol) and DIEA (163 mg, 1.26 mmol) under nitrogen atmosphere and stirred at 80° C. for 16 h. The reaction mixture was cooled to rt, diluted with water (30 mL) and extracted with DCM (20 mL) for three times. The combined organic layers were dried over Na2SO4, filtered and concentrated in vacuo to give a residue which was purified by flash (DCM/MeOH=10/1) to afford (2R,4S)-tert-butyl 4-(4-amino-3-((1-cyclopropyl-2-methyl-1H-benzo[d]imidazol-5-yl)ethynyl)-1H-pyrazolo[3,4-d]pyrimidin-1-yl)-2-(methoxymethyl)pyrrolidine-1-carboxylate (180 mg, 79%) as a white solid. [M+H] Calcd.: 543.3; Found, 543.3.

### Step 7: TFA salt of 3-((1-cyclopropyl-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine

To a solution of (2R,4S)-tert-butyl 4-(4-amino-3-((1-cyclopropyl-2-methyl-1H-benzo[d]imidazol-5-yl)ethynyl)-1H-pyrazolo[3,4-d]pyrimidin-1-yl)-2-(methoxymethyl)pyrrolidine-1-carboxylate (180 mg, 0.33 mmol) in DCM (10 mL) was added TFA (0.5 mL). The reaction mixture was stirred at room temperature for 1 h. The reaction mixture was concentrated in vacuo to afford TFA salt of 3-((1-cyclopropyl-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine (146 mg, 100%) as yellow oil. [M+H] Calcd.: 443.2; Found, 443.2.

### Step 8: 1-((2R,4S)-4-(4-amino-3-((1-cyclopropyl-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of TFA salt of 3-((1-cyclopropyl-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine (146 mg, 0.33 mmol) and DIEA (128 mg, 0.99 mmol) in DCM (15 mL) at −40° C. under nitrogen atmosphere was added a solution of acryloyl chloride (30 mg, 0.33 mmol) in DCM (0.5 mL) slowly. The mixture was stirred at −40° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((1-cyclopropyl-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (49.3 mg, 30%) as a white solid. 1H NMR (400 MHz, CDCl3): 8.38 (s, 1H), 7.89 (s, 1H), 7.50-7.47 (m, 2H), 6.54-6.39 (m, 2H), 5.97 (br s, 2H), 5.81-5.66 (m, 2H), 4.66-4.46 (m, 1H), 4.13-4.06 (m, 2H), 3.81-3.79 (m, 1H), 3.54-3.51 (m, 1H), 3.39 (s, 3H), 3.27-3.24 (m, 1H), 2.94-2.75 (m, 1H), 2.70 (s, 3H), 2.47-2.44 (m, 1H), 1.29-1.27 (m, 2H), 1.08-1.07 (m, 2H). [M+H] Calcd.: 497.3; Found, 497.3.

### Example 16: 1-((2R,4S)-4-(4-amino-3-((4,6-difluoro-1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

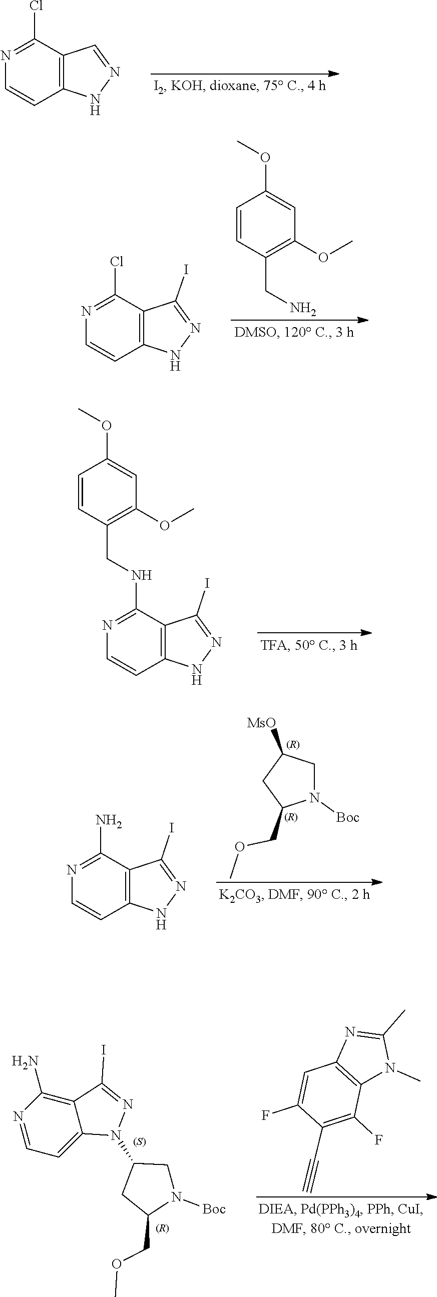

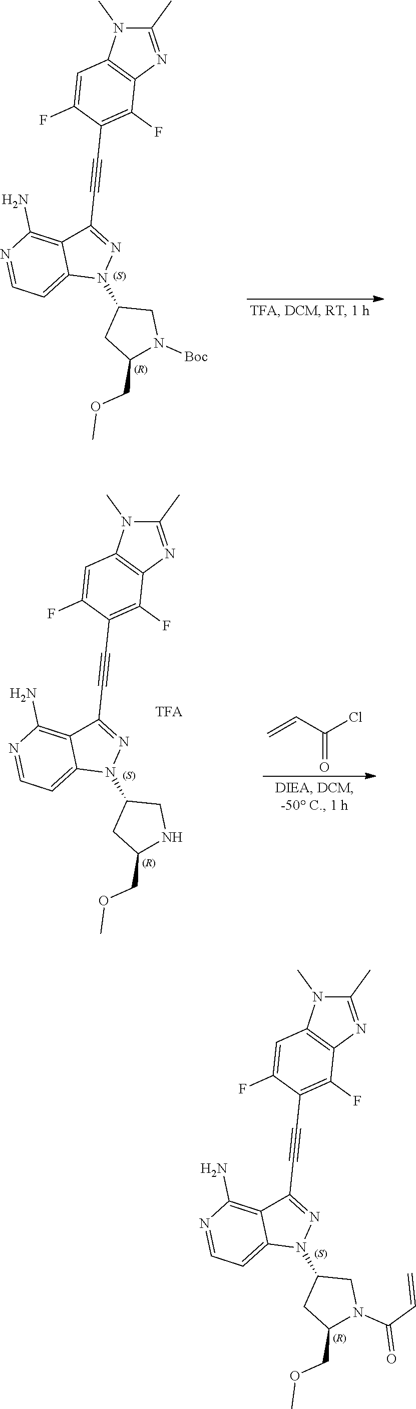

### Step 1: 4-chloro-3-iodo-1H-pyrazolo[4,3-c]pyridine

A solution of 4-chloro-1H-pyrazolo[4,3-c]pyridine (1.5 g, 9.8 mmol), 1,4-dioxane (25 mL), potassium hydroxide (2.0 g, 35.7 mmol) and iodine (4.9 g, 19.5 mmol) was stuffed for 4 h at 75° C. The reaction was The reaction mixture was quenched with saturated aqueous sodium thiosulfate pentahydrate (30 mL) and the solids were collected by filtration to give 4-chloro-3-iodo-1H-pyrazolo[4,3-c]pyridine (2.5 g, 92%) as a yellow solid. [M+H] Calcd.: 280.0; Found, 280.0.

### Step 2: N-(2,4-dimethoxybenzyl)-3-iodo-1H-pyrazolo[4,3-c]pyridin-4-amine

To a solution of 4-chloro-3-iodo-1H-pyrazolo[4,3-c]pyridine (8.5 g, 30.0 mmol) in DMSO (100 mL) was added (2,4-dimethoxyphenyl)methanamine (15.3 g, 90.0 mmol). The mixture was heated at 120° C. for 3 h. The mixture was cooled to rt, diluted EA (200 mL), washed with water (100 mL) and brine (100 mL), dried over Na2SO4, filtered and concentrated to afford N-(2,4-dimethoxybenzyl)-3-iodo-1H-pyrazolo[4,3-c]pyridin-4-amine (8.6 g, 70%) as a yellow oil which was used for next step without further purification. [M+H] Calcd.: 411.0; Found, 411.0.

### Step 3: 3-iodo-1H-pyrazolo[4,3-c]pyridin-4-amine

A mixture of N-(2,4-dimethoxybenzyl)-3-iodo-1H-pyrazolo[4,3-c]pyridin-4-amine (8.6 g, 21.0 mmol) in TFA (50 mL) was heated to 50° C. for 3 h. After the solvent was removed, the residue was basified to pH=8 with sodium bicarbonate aqueous solution and extracted with EA (100 mL) for three times. The combined organic layers were washed with brine (100 mL), dried over anhydrous sodium sulfate, filtered and evaporated under vacuum. The residue was purified by column chromatography (PE/EA=3/1) to afford 3-iodo-1H-pyrazolo[4,3-c]pyridin-4-amine (4.3 g, 60%) as a white solid. [M+H] Calcd.: 260.7; Found, 260.7.

### Step 4: (2R,4S)-tert-butyl 4-(4-amino-3-iodo-1H-pyrazolo[4,3-c]pyridin-1-yl)-2-(methoxymethyl)pyrrolidine-1-carboxylate

To a solution of 3-iodo-1H-pyrazolo[4,3-c]pyridin-4-amine (3.8 g, 14.7 mmol) and crude (2R,4R)-tert-butyl 2-(methoxymethyl)-4-((methylsulfonyl)oxy)pyrrolidine-1-carboxylate (3.4 g, 14.7 mmol) in DMF (50 mL) was added K2CO3 (6.0 g, 34.9 mmol). The reaction mixture was stirred at 90° C. for 2 h. The reaction mixture was cooled to rt, quenched with ice water (100 mL) and extracted with DCM (100 mL) for three times. The combined organic layers were washed with brine, dried over sodium sulfate, filtered and concentrated to afford a residue which was purified by silica gel column chromatography (DCM/MeOH=30/1) to afford (2R,4S)-tert-butyl 4-(4-amino-3-iodo-1H-pyrazolo[4,3-c]pyridin-1-yl)-2-(methoxymethyl)pyrrolidine-1-carboxylate (3.4 g, 50%) as a yellow solid. [M+H] Calcd.: 474.1; Found, 474.1.

### Step 5: (2R,4S)-tert-butyl 4-(4-amino-3-((4,6-difluoro-1,2-dimethyl-1H-benzo[d]imidazol-5-yl)ethynyl)-1H-pyrazolo[4,3-c]pyridin-1-yl)-2-(methoxymethyl)pyrrolidine-1-carboxylate

To a solution of (2R,4S)-tert-butyl 4-(4-amino-3-iodo-1H-pyrazolo[4,3-c]pyridin-1-yl)-2-(methoxymethyl)pyrrolidine-1-carboxylate (300 mg, 0.63 mmol), 6-ethynyl-5,7-difluoro-1,2-dimethyl-1H-benzo[d]imidazole (155 mg, 0.76 mmol) and CuI (18 mg, 0.09 mmol) in DMF (20 mL) was added Pd(PPh3)4 (364 mg, 0.32 mmol), PPh3 (8 mg, 0.03 mmol) and DIEA (244 mg, 1.89 mmol) under nitrogen atmosphere and stirred at 80° C. for 16 h. The reaction mixture was cooled to rt, diluted with water (30 mL) and extracted with DCM (20 mL) for three times. The combined organic layers were dried over Na2SO4, filtered and concentrated in vacuo to give a residue which was purified by flash (DCM/MeOH=20/1) to afford (2R,4S)-tert-butyl 4-(4-amino-3-((4,6-difluoro-1,2-dimethyl-1H-benzo[d]imidazol-5-yl)ethynyl)-1H-pyrazolo[4,3-c]pyridin-1-yl)-2-(methoxymethyl)pyrrolidine-1-carboxylate (200 mg, 57%) as a white solid. [M+H] Calcd.: 552.2; Found, 552.2.

### Step 6: TFA salt of 3-((4,6-difluoro-1,2-dimethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine

To a solution of (2R,4S)-tert-butyl 4-(4-amino-3-((4,6-difluoro-1,2-dimethyl-1H-benzo[d]imidazol-5-yl)ethynyl)-1H-pyrazolo[4,3-c]pyridin-1-yl)-2-(methoxymethyl)pyrrolidine-1-carboxylate (200 mg, 0.37 mmol) in DCM (10 mL) was added TFA (0.5 mL). The reaction mixture was stirred at room temperature for 1 h. The reaction mixture was concentrated in vacuo to afford TFA salt of 3-((4,6-difluoro-1,2-dimethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine (163 mg, 100%) as yellow oil. [M+H] Calcd.: 452.2; Found, 452.2.

### Step 7: 1-((2R,4S)-4-(4-amino-3-((4,6-difluoro-1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of TFA salt of 3-((4,6-difluoro-1,2-dimethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine (163 mg, 0.36 mmol) and DIEA (139 mg, 1.08 mmol) in DCM (15 mL) at −50° C. under nitrogen atmosphere was added a solution of acryloyl chloride (33 mg, 0.36 mmol) in DCM (0.5 mL) slowly. The mixture was stirred at −50° C. for 1 h. The reaction mixture was cooled to rt, quenched with water (15 mL) and extracted with DCM (10 mL) twice. The combined organic layers were dried over Na2SO4, filtered and concentrated in vacuo to give a residue which was purified by prep-HPLC to afford a HCOOH salt of 1-((2R,4S)-4-(4-amino-3-((4,6-difluoro-1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (39.1 mg, 21%) as a white solid. 1H NMR (400 MHz, DMSO-d6): 8.13 (s, 1H), 7.81 (d, J=6.0 Hz, 1H), 7.59 (d, J=9.6 Hz, 1H), 7.00 (d, J=6.4 Hz, 1H), 6.77-6.60 (m, 3H), 6.18-6.13 (m, 1H), 5.71-5.65 (m, 1H), 5.50-5.48 (m, 1H), 4.59-4.48 (m, 1H), 4.04-3.75 (m, 5H), 3.60-3.47 (m, 2H), 2.69-2.55 (m, 1H), 2.51-2.49 (m, 7H). [M+H] Calcd.: 506.2; Found, 506.2.

### Example 17: 1-((2R,4S)-4-(4-amino-3-((1-cyclopropyl-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

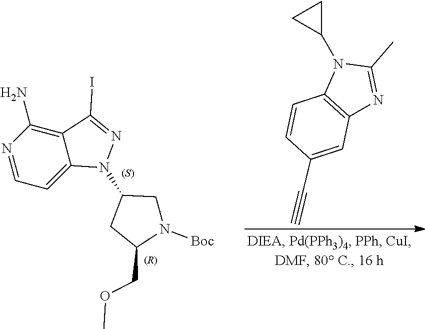

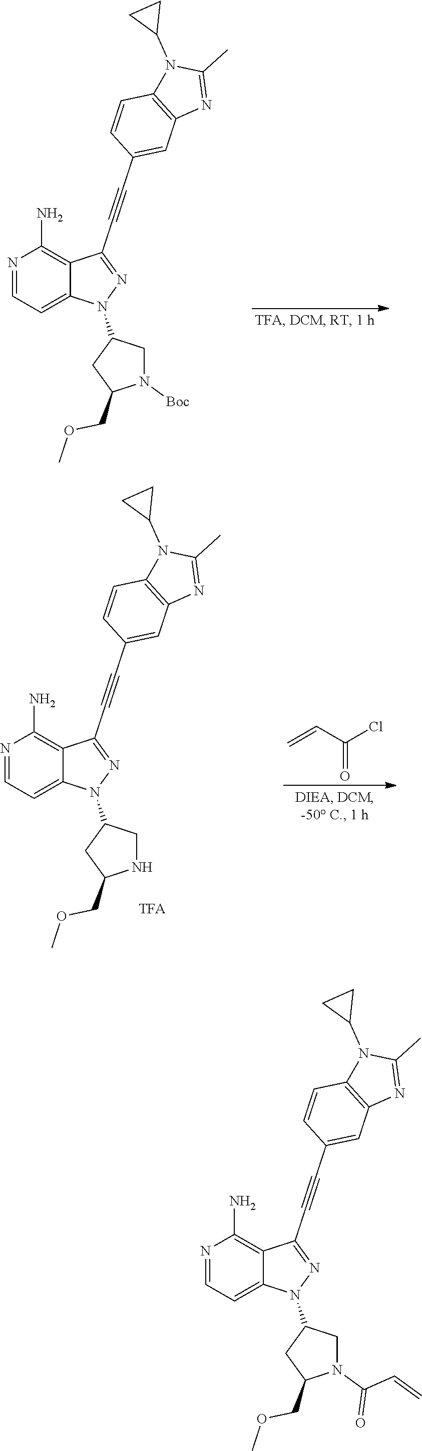

### Step 1: (2R,4S)-tert-butyl 4-(4-amino-3-((1-cyclopropyl-2-methyl-1H-benzo[d]imidazol-5-yl)ethynyl)-1H-pyrazolo[4,3-c]pyridin-1-yl)-2-(methoxymethyl)pyrrolidine-1-carboxylate

To a solution of (2R,4S)-tert-butyl 4-(4-amino-3-iodo-1H-pyrazolo[4,3-c]pyridin-1-yl)-2-(methoxymethyl)pyrrolidine-1-carboxylate (125 mg, 0.26 mmol), 1-cyclopropyl-5-ethynyl-2-methyl-1H-benzo[d]imidazole (61 mg, 0.32 mmol) and CuI (7 mg, 0.04 mmol) in DMF (20 mL) was added Pd(PPh3)4 (151 mg, 0.13 mmol), PPh3 (3 mg, 0.01 mmol) and DIEA (102 mg, 0.79 mmol) under nitrogen atmosphere and stirred at 80° C. for 16 h. The reaction mixture was cooled to rt, diluted with water (30 mL) and extracted with DCM (20 mL) for three times. The combined organic layers were dried over Na2SO4, filtered and concentrated in vacuo to give a residue which was purified by flash (DCM/MeOH=20/1) to afford (2R,4S)-tert-butyl 4-(4-amino-3-((1-cyclopropyl-2-methyl-1H-benzo[d]imidazol-5-yl)ethynyl)-1H-pyrazolo[4,3-c]pyridin-1-yl)-2-(methoxymethyl)pyrrolidine-1-carboxylate (65 mg, 28%) as a white solid. [M+H] Calcd.: 542.3; Found, 542.3.

### Step 2: TFA salt of 3-((1-cyclopropyl-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine

To a solution of (2R,4S)-tert-butyl 4-(4-amino-3-((1-cyclopropyl-2-methyl-1H-benzo[d]imidazol-5-yl)ethynyl)-1H-pyrazolo[4,3-c]pyridin-1-yl)-2-(methoxymethyl)pyrrolidine-1-carboxylate (65 mg, 0.12 mmol) in DCM (10 mL) was added TFA (0.5 mL). The reaction mixture was stirred at room temperature for 1 h. The reaction mixture was concentrated in vacuo to afford TFA salt of 3-((1-cyclopropyl-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine (53 mg, 100%) as yellow oil. [M+H] Calcd.: 442.2; Found, 442.2.

### Step 3: 1-((2R,4S)-4-(4-amino-3-((1-cyclopropyl-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of TFA salt of 3-((1-cyclopropyl-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine (53 mg, 0.12 mmol) and DIEA (45 mg, 0.35 mmol) in DCM (15 mL) at −50° C. under nitrogen atmosphere was added a solution of acryloyl chloride (10 mg, 0.11 mmol) in DCM (0.5 mL) slowly. The mixture was stirred at −50° C. for 1 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((1-cyclopropyl-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (16.6 mg, 28%) as a white solid. 1H NMR (400 MHz, DMSO-d6): 7.85 (s, 1H), 7.79 (d, J=6.0 Hz, 1H), 7.59 (d, J=8.4 Hz, 1H), 7.49-7.46 (m, 1H), 6.95 (d, J=6.4 Hz, 1H), 6.76-6.53 (m, 1H), 6.37 (s, 2H), 6.17-6.13 (m, 1H), 5.72-5.64 (m, 1H), 5.49-5.46 (m, 1H), 4.58-4.45 (m, 1H), 4.07-3.78 (m, 2H), 3.60-3.33 (m, 6H), 2.60-2.50 (m, 4H), 2.41-2.38 (m, 1H), 1.21-1.96 (m, 2H), 1.04-1.02 (m, 2H). [M+H] Calcd.: 496.2; Found, 496.2.

### Example 18: 1-((2R,4S)-4-(4-amino-3-((5,7-difluoro-2-methyl-1H-benzo[d]imidazol-6-yl)ethynyl)-1H-pyrazolo[4,3-c]pyridin-1-yl)-2-(methoxymethyl)pyrrolidin-1-yl)prop-2-en-1-one

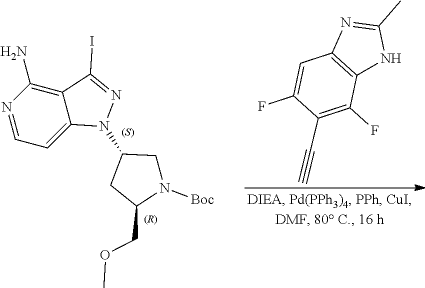

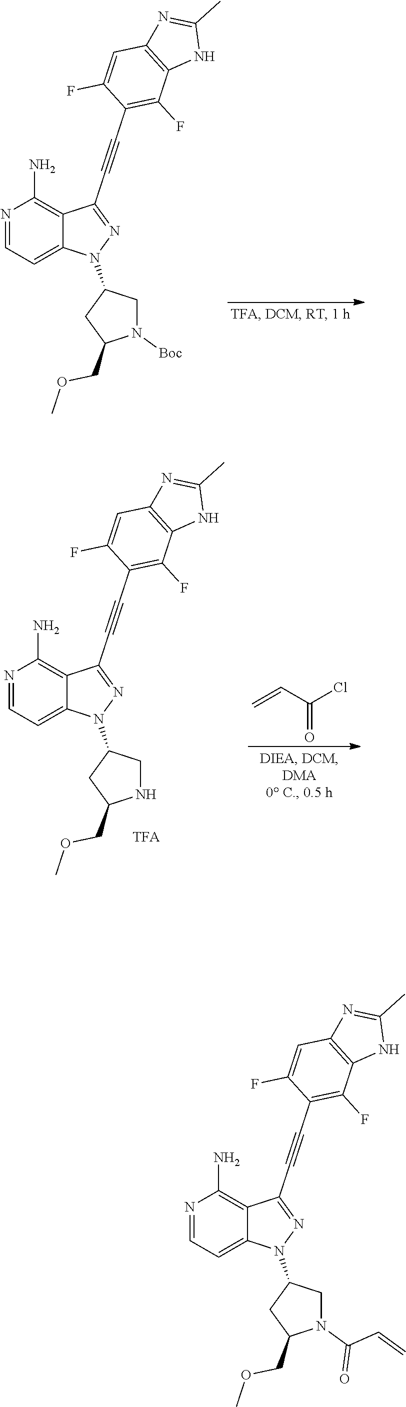

### Step 1: (2R,4S)-tert-butyl 4-(4-amino-3-((5,7-difluoro-2-methyl-1H-benzo[d]imidazol-6-yl)ethynyl)-1H-pyrazolo[4,3-c]pyridin-1-yl)-2-(methoxymethyl)pyrrolidine-1-carboxylate

To a solution of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.42 mmol), 6-ethynyl-5,7-difluoro-2-methyl-1H-benzo[d]imidazole (97 mg, 0.51 mmol) and CuI (11 mg, 0.06 mmol) in DMF (15 mL) was added Pd(PPh3)4 (243 mg, 0.21 mmol), PPh3 (6 mg, 0.02 mmol) and DIEA (163 mg, 1.26 mmol) under nitrogen atmosphere and stirred at 80° C. for 16 h. The reaction mixture was cooled to rt, diluted with water (30 mL) and extracted with DCM (20 mL) for three times. The combined organic layers were dried over Na2SO4, filtered and concentrated in vacuo to give a residue which was purified by flash (DCM/MeOH=20/1) to afford (2R,4S)-tert-butyl 4-(4-amino-3-((5,7-difluoro-2-methyl-1H-benzo[d]imidazol-6-yl)ethynyl)-1H-pyrazolo[4,3-c]pyridin-1-yl)-2-(methoxymethyl)pyrrolidine-1-carboxylate (80 mg, 35%) as a white solid. [M+H] Calcd.: 538.2; Found, 538.2.

### Step 2: TFA salt of 3-((5,7-difluoro-2-methyl-1H-benzo[d]imidazol-6-yl)ethynyl)-1-((3S,5R)-5-(methoxymethyl)pyrrolidin-3-yl)-1H-pyrazolo[4,3-c]pyridin-4-amine

To a solution of (2R,4S)-tert-butyl 4-(4-amino-3-((5,7-difluoro-2-methyl-1H-benzo[d]imidazol-6-yl)ethynyl)-1H-pyrazolo[4,3-c]pyridin-1-yl)-2-(methoxymethyl)pyrrolidine-1-carboxylate (80 mg, 0.15 mmol) in DCM (10 mL) was added TFA (0.5 mL). The reaction mixture was stirred at room temperature for 1 h. The reaction mixture was concentrated in vacuo to afford TFA salt of 3-((5,7-difluoro-2-methyl-1H-benzo[d]imidazol-6-yl)ethynyl)-1-((3S,5R)-5-(methoxymethyl)pyrrolidin-3-yl)-1H-pyrazolo[4,3-c]pyridin-4-amine (65 mg, 100%) as yellow oil. [M+H] Calcd.: 438.2; Found, 438.2.

### Step 3: 1-((2R,4S)-4-(4-amino-3-((5,7-difluoro-2-methyl-1H-benzo[d]imidazol-6-yl)ethynyl)-1H-pyrazolo[4,3-c]pyridin-1-yl)-2-(methoxymethyl)pyrrolidin-1-yl)prop-2-en-1-one

To a solution of TFA salt of 3-((5,7-difluoro-2-methyl-1H-benzo[d]imidazol-6-yl)ethynyl)-1-((3S,5R)-5-(methoxymethyl)pyrrolidin-3-yl)-1H-pyrazolo[4,3-c]pyridin-4-amine (65 mg, 0.15 mmol) and DIEA (45 mg, 0.35 mmol) in DCM (15 mL) and DMA (2 mL) at 0° C. under nitrogen atmosphere was added a solution of acryloyl chloride (10 mg, 0.11 mmol) in DCM (0.5 mL) slowly. The mixture was stirred at 0° C. for 0.5 h. The reaction mixture was quenched with water (15 mL) and extracted with DCM (10 mL) twice. The combined organic layers were dried over Na2SO4, filtered and concentrated in vacuo to give a residue which was purified by prep-HPLC to afford 1-((2R,4S)-4-(4-amino-3-((5,7-difluoro-2-methyl-1H-benzo[d]imidazol-6-yl)ethynyl)-1H-pyrazolo[4,3-c]pyridin-1-yl)-2-(methoxymethyl)pyrrolidin-1-yl)prop-2-en-1-one (22.1 mg, 30%) as a white solid. 1H NMR (400 MHz, DMSO-d6): 13.21-12.91 (m, 1H), 7.81 (d, J=6.0 Hz, 1H), 7.39 (s, 1H), 6.97 (d, J=6.4 Hz, 1H), 6.77-6.50 (m, 3H), 6.18-6.14 (m, 1H), 5.69-5.66 (m, 1H), 5.52-5.48 (m, 1H), 4.60-4.47 (m, 1H), 4.08-3.77 (m, 2H), 3.60-3.54 (m, 2H), 3.33 (s, 3H), 2.67-2.50 (m, 4H), 2.42-2.38 (m, 1H). [M+H] Calcd.: 492.2; Found, 492.2.

### Example 19: 1-((2R,4S)-4-(4-amino-3-((1-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

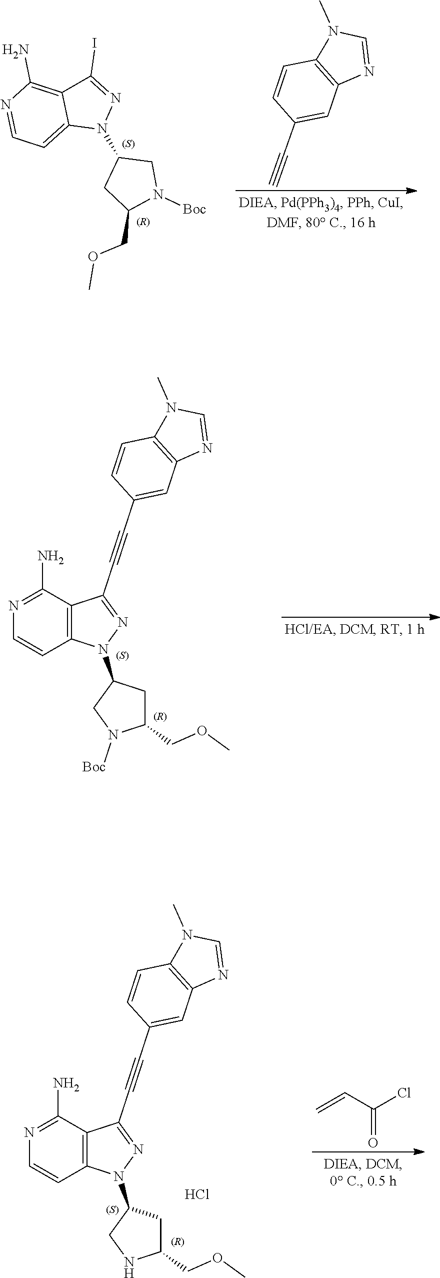

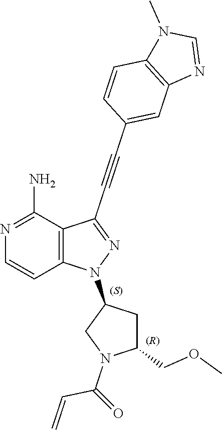

### Step 1: (R,4S)-tert-butyl 4-(4-amino-3-((1-methyl-1H-benzo[d]imidazol-5-yl)ethynyl)-1H-pyrazolo[4,3-c]pyridin-1-yl)-2-(methoxymethyl)pyrrolidine-1-carboxylate

To a solution of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.42 mmol), 5-ethynyl-1-methyl-1H-benzo[d]imidazole (66 mg, 0.42 mmol) and CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 3 h. The reaction mixture was cooled to rt, diluted with water (20 mL) and extracted with DCM (30 mL) for three times. The combined organic layers were dried over Na2SO4, filtered and concentrated in vacuo. The residue was purified by flash (DCM/MeOH=20/1) to afford (R,4S)-tert-butyl 4-(4-amino-3-((1-methyl-1H-benzo[d]imidazol-5-yl)ethynyl)-1H-pyrazolo[4,3-c]pyridin-1-yl)-2-(methoxymethyl)pyrrolidine-1-carboxylate (140 mg, 66%) as a yellow solid. [M+H] Calcd.: 502.2; Found, 502.2.

### Step 2: HCl salt of 1-((3S,5R)-5-(methoxymethyl)pyrrolidin-3-yl)-3-((1-methyl-1H-benzo[d]imidazol-5-yl)ethynyl)-1H-pyrazolo[4,3-c]pyridin-4-amine

To a solution of (R,4S)-tert-butyl 4-(4-amino-3-((1-methyl-1H-benzo[d]imidazol-5-yl)ethynyl)-1H-pyrazolo[4,3-c]pyridin-1-yl)-2-(methoxymethyl)pyrrolidine-1-carboxylate (140 mg, 0.28 mmol) in EA (5 mL) was added HCl/EA (15 mL) at room temperature. The reaction mixture was stirred at room temperature for 2 h. LCMS showed the reaction was completed. The reaction mixture was concentrated under vacuum to a crude HCl salt of 1-((3S,5R)-5-(methoxymethyl)pyrrolidin-3-yl)-3-((1-methyl-1H-benzo[d]imidazol-5-yl)ethynyl)-1H-pyrazolo[4,3-c]pyridin-4-amine (130 mg, 100%) as a yellow solid. [M+H] Calcd.: 402.2; Found, 402.2.

### Step 3: 1-((2R,4S)-4-(4-amino-3-((1-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of HCl salt of 1-((3S,5R)-5-(methoxymethyl)pyrrolidin-3-yl)-3-((1-methyl-1H-benzo[d]imidazol-5-yl)ethynyl)-1H-pyrazolo[4,3-c]pyridin-4-amine (105 mg, 0.24 mmol) and DIEA (102 mg, 0.72 mmol) in DCM (2 mL) and DMA (2 mL) at −50° C. under nitrogen atmosphere was added a solution of acryloyl chloride (24 mg, 0.26 mmol) in DCM (0.5 mL) slowly. The mixture was stirred at −50° C. for 0.5 h. The reaction mixture was quenched with water (5 mL) and extracted with DCM (5 mL) twice. The combined organic layers were dried over Na2SO4, filtered and concentrated in vacuo to give a residue which was purified by prep-HPLC to afford 1-((2R,4S)-4-(4-amino-3-((1-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (52.7 mg, 30%) as a yellow solid. 1H NMR (400 MHz, CDCl3): 8.49 (s, 1H), 8.08 (s, 1H), 7.99 (s, 1H), 7.63-7.44 (m, 3H), 6.80 (d, J=8.4 Hz, 1H), 6.51-6.42 (m, 2H), 5.74 (dd, J=4.8, 8.0 Hz, 1H), 5.54-5.31 (m, 1H), 4.68-4.51 (m, 1H), 4.16-4.04 (m, 2H), 3.95-3.92 (m, 1H), 3.91 (s, 3H), 3.56-3.55 (m, 1H), 3.56-3.40 (m, 1H), 3.40 (s, 3H), 2.79-2.71 (m, 1H), 2.47-2.42 (m, 1H). [M+H] Calcd.: 456.2; Found, 456.2.

### Example 20: 1-((2R,4S)-4-(4-amino-3-((1-ethyl-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

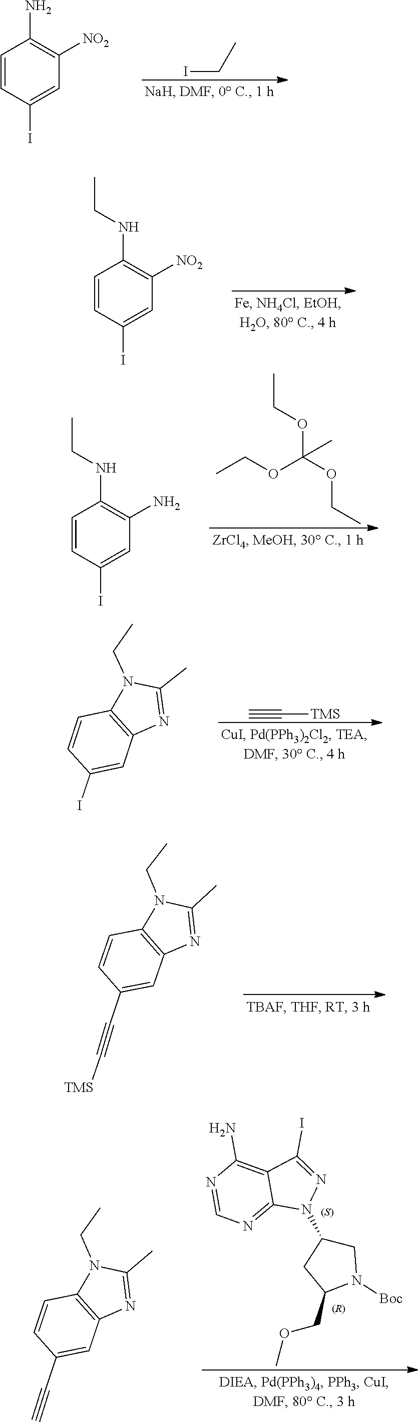

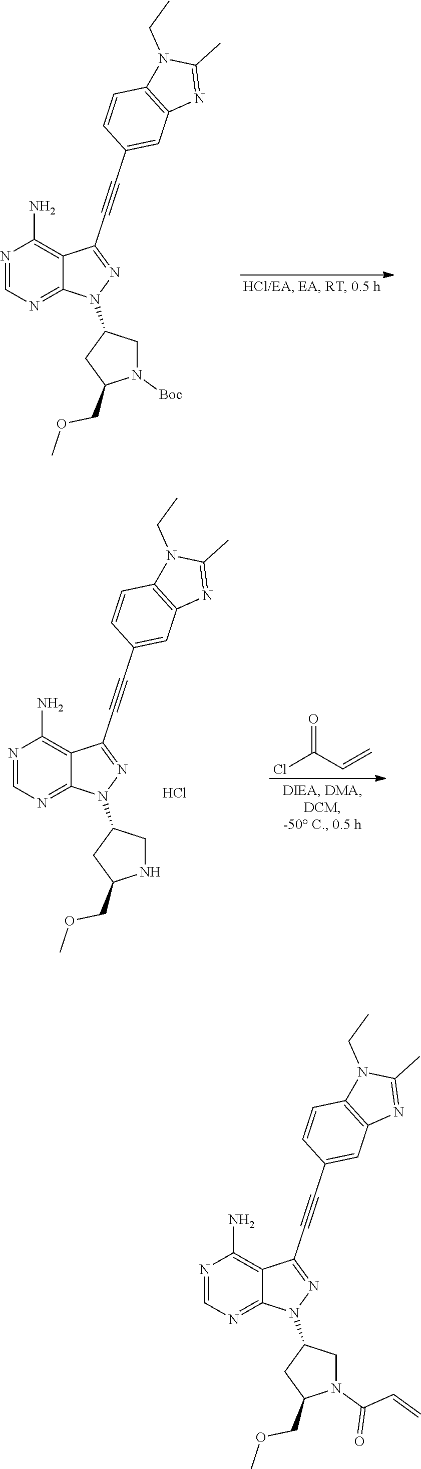

### Step 1: N-ethyl-4-iodo-2-nitroaniline

To a stirred solution of 4-iodo-2-nitroaniline (10.0 g, 37.9 mmol) in anhydrous DMF (80 mL) was added 60% wt. sodium hydride in mineral oil (1.5 g, 37.9 mmol) in portions at 0° C. under nitrogen atmosphere. The reaction mixture was stirred at 0° C. for 30 min and then iodoethane (5.9 g, 37.9 mmol) was added and stirred at 0° C. for 30 min. After The reaction mixture was quenched with water (240 mL) and extracted with EA (100 mL) for three times. The combined organic layers were dried over Na2SO4, filtered and concentrated in vacuo to afford N-ethyl-4-iodo-2-nitroaniline (10.8 g, 98%) as a red solid. [M+H] Calcd.: 293.1; Found, 293.1.

### Step 2: N1-ethyl-4-iodobenzene-1,2-diamine

To a solution of N-ethyl-4-iodo-2-nitroaniline (5.0 g, 1.7 mmol) in EtOH (150 mL) and H2O (30 mL) was added Fe (3.8 g, 68.4 mmol) and NH4C1 (3.6 g, 68.4 mmol) at rt. Then the mixture was stirred at 80° C. for 4 h. After cooling down to rt, the reaction mixture was filtered, and the filtrate was extracted with EA (20 mL) for three times. The combined organic layers were washed with brine (20 mL), dried over Na2SO4, filtered and concentrated. The residue was purified by flash (DCM/MeOH=50/1) to give N1-ethyl-4-iodobenzene-1,2-diamine (4.25 g, 95%) as a black solid. [M+H] Calcd.: 263.1; Found, 263.1.

### Step 3: 1-ethyl-5-iodo-2-methyl-1H-benzo[d]imidazole

A mixture of N1-ethyl-4-iodobenzene-1,2-diamine (3.2 g, 12.2 mmol), 1,1,1-triethoxyethane (2.4 g, 14.8 mmol) and ZrCl4 (280 mg, 1.22 mmol) in MeOH (20 mL) was stirred at 30° C. for 1 h. Then the reaction mixture was dissolved in water (30 mL) and extracted with DCM (15 mL) for three times. The combined organic layers were washed with brine (20 mL), dried over Na2SO4, filtered and concentrated. The residue was purified by flash (PE/EA=5/1) to give 1-ethyl-5-iodo-2-methyl-1H-benzo[d]imidazole (2.50 g, 72%) as a red solid. [M+H] Calcd.: 287.0; Found, 287.0.

### Step 4: 1-ethyl-2-methyl-5-((trimethylsilyl)ethynyl)-1H-benzo[d]imidazole

To a solution of 1-ethyl-5-iodo-2-methyl-1H-benzo[d]imidazole (2.5 g, 8.7 mmol), ethynyltrimethylsilane (1.7 g, 17.4 mmol) and CuI (247 mg, 1.30 mmol) in DMF (15 mL) was added Pd(PPh3)2Cl2 (308 mg, 0.44 mmol) and TEA (1.3 g, 13.1 mmol) under nitrogen atmosphere. The reaction mixture was stirred at 30° C. for 4 h. The reaction mixture was cooled to rt, diluted with water (30 mL) and extracted with DCM (20 mL) for three times. The combined organic layers were dried over Na2SO4, filtered and concentrated in vacuo to give a residue which was purified by flash (DCM/MeOH=50/1) to afford 1-ethyl-2-methyl-5-((trimethylsilyl)ethynyl)-1H-benzo[d]imidazole (2.0 g, 93%) as a brown solid. [M+H] Calcd.: 257.2; Found, 257.2.

### Step 5: 1-ethyl-5-ethynyl-2-methyl-1H-benzo[d]imidazole

To a solution of 1-ethyl-2-methyl-5-((trimethylsilyl)ethynyl)-1H-benzo[d]imidazole (2.0 g, 7.8 mmol) in THF (20 mL) was added a solution of TBAF in THF (7.8 mL, 7.8 mmol). Then the mixture was stirred at rt for 3 h. The reaction mixture was quenched with water (40 mL) and extracted with EA (30 mL*3). The combined organic layers were washed with brine (20 mL), dried over Na2SO4, filtered and concentrated. The residue was purified by flash (DCM/MeOH=50/1) to afford 1-ethyl-5-ethynyl-2-methyl-1H-benzo[d]imidazole (1.4 g, 96%) as a red solid. [M+H] Calcd.: 185.1; Found, 185.1.

### Step 6: (2R,4S)-tert-butyl 4-(4-amino-3-((1-ethyl-2-methyl-1H-benzo[d]imidazol-5-yl)ethynyl)-1H-pyrazolo[3,4-d]pyrimidin-1-yl)-2-(methoxymethyl)pyrrolidine-1-carboxylate

To a solution of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.40 mmol), 1-ethyl-5-ethynyl-2-methyl-1H-benzo[d]imidazole (81 mg, 0.44 mmol) and CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 3 h. The reaction mixture was cooled to rt, diluted with water (20 mL) and extracted with DCM (30 mL) for three times. The combined organic layers were dried over Na2SO4, filtered and concentrated in vacuo to afford (2R,4S)-tert-butyl 4-(4-amino-3-((1-ethyl-2-methyl-1H-benzo[d]imidazol-5-yl)ethynyl)-1H-pyrazolo[3,4-d]pyrimidin-1-yl)-2-(methoxymethyl)pyrrolidine-1-carboxylate (200 mg, 94%) as a brown solid. [M+H] Calcd.: 531.2; Found, 531.2.

### Step 7: HCl salt of 3-((1-ethyl-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine

To a solution of (2R,4S)-tert-butyl 4-(4-amino-3-((1-ethyl-2-methyl-1H-benzo[d]imidazol-5-yl)ethynyl)-1H-pyrazolo[3,4-d]pyrimidin-1-yl)-2-(methoxymethyl)pyrrolidine-1-carboxylate (200 mg, 0.38 mmol) in EA (5 mL) was added HCU/EA (15 mL) at room temperature. The reaction mixture was stirred at room temperature for 0.5 h. LCMS showed the reaction was completed. The reaction mixture was concentrated under vacuum to a crude HCl salt of 3-((1-ethyl-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine (170 mg, 97%) as a yellow solid. [M+H] Calcd.: 431.2; Found, 431.2.

### Step 8: 1-((2R,4S)-4-(4-amino-3-((1-ethyl-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of HCl salt of 3-((1-ethyl-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine (170 mg, 0.36 mmol) and DIEA (140 mg, 1.08 mmol) in DCM (2 mL) and DMA (2 mL) at −50° C. under nitrogen atmosphere was added a solution of acryloyl chloride (33 mg, 0.36 mmol) in DCM (0.5 mL) slowly. The mixture was stirred at −50° C. for 0.5 h. The reaction mixture was quenched with water (5 mL) and extracted with DCM (5 mL) twice. The combined organic layers were dried over Na2SO4, filtered and concentrated in vacuo to give a residue which was purified by prep-HPLC to afford formate salt of 1-((2R,4S)-4-(4-amino-3-((1-ethyl-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (52.7 mg, 30%) as a yellow solid. 1H NMR (400 MHz, CDCl3): 8.36 (s, 1H), 7.94 (s, 1H), 7.48 (d, J=8.0 Hz, 1H), 7.32 (d, J=8.4 Hz, 1H), 6.45-6.40 (m, 2H), 6.18 (br s, 2H), 5.85-5.67 (m, 2H), 4.67-4.65 (m, 1H), 4.22-4.04 (m, 4H), 3.82 (dd, J=4.0, 9.2 Hz, 1H), 3.54-3.49 (m, 1H), 3.49 (s, 3H), 2.83-2.76 (m, 1H), 2.65 (s, 3H), 2.50-2.45 (m, 1H), 1.44 (t, J=7.2 Hz, 3H). [M+H] Calcd.: 485.3; Found, 485.3.

### Example 21: 1-((2R,4S)-4-(4-amino-3-((1-ethyl-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

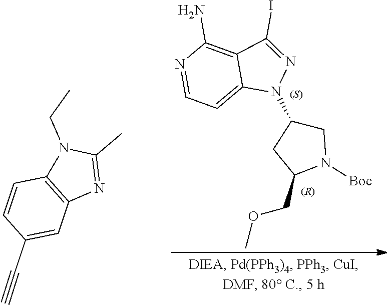

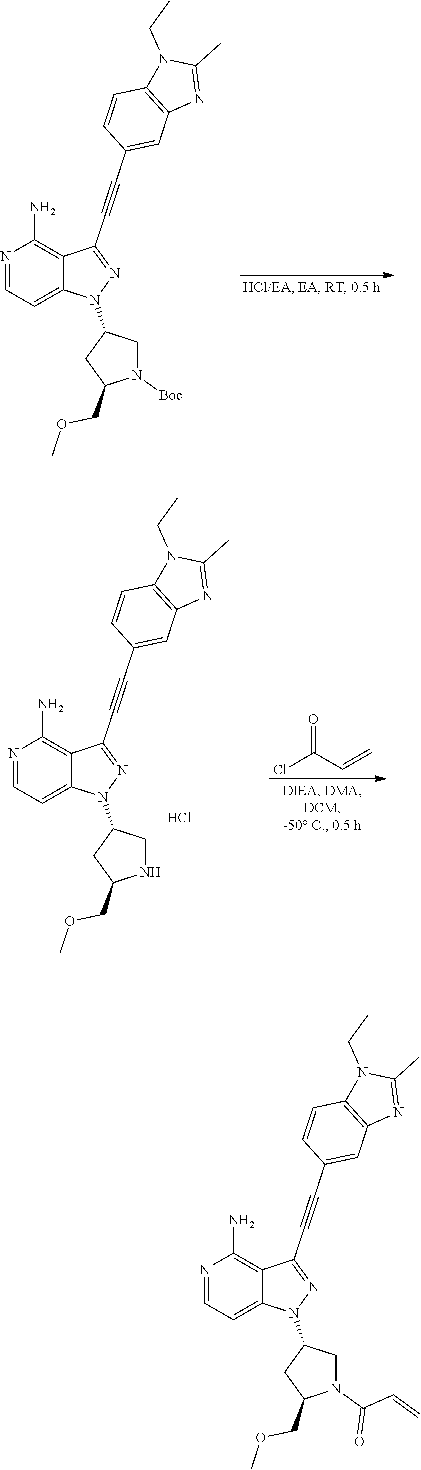

### Step 1: (2R,4S)-tert-butyl 4-(4-amino-3-((1-ethyl-2-methyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.40 mmol), 1-ethyl-5-ethynyl-2-methyl-1H-benzo[d]imidazole (74 mg, 0.40 mmol), CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 5 h. After cooling down to room temperature, the reaction mixture was diluted with water (20 mL) and extracted with DCM (30 mL) for three times, the organic layers were dried over Na2SO4, filtered and concentrated in vacuo to afford ((2R,4S)-tert-butyl 4-(4-amino-3-((1-ethyl-2-methyl-1H-benzo[d]imidazol-5-yl)ethynyl)-1H-pyrazolo[4,3-c]pyridin-1-yl)-2-(methoxymethyl)pyrrolidine-1-carboxylate (140 mg, 66%) as a brown solid. [M+H] Calcd.: 530.2; Found, 530.2.

### Step 2: 3-((1-ethyl-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine hydrochloride

To a solution of ((2R,4S)-tert-butyl 4-(4-amino-3-((1-ethyl-2-methyl-1H-benzo[d]imidazol-5-yl)ethynyl)-1H-pyrazolo[4,3-c]pyridin-1-yl)-2-(methoxymethyl)pyrrolidine-1-carboxylate (140 mg, 0.26 mmol) in EA (5 mL) was added HCl/EA (15 mL) at room temperature. The reaction mixture was stirred at room temperature for 0.5 h. The reaction mixture was concentrated under vacuum to give 3-((1-ethyl-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine hydrochloride (112 mg, 93%) as a yellow solid. [M+H] Calcd.: 430.2; Found, 430.2.

### Step 3: 1-((2R,4S)-4-(4-amino-3-((1-ethyl-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of 3-((1-ethyl-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine hydrochloride (112 mg, 0.20 mmol), DIEA (78 mg, 0.60 mmol) in DCM (2 mL) and DMA (2 mL) at −50° C. under nitrogen atmosphere was added acryloyl chloride (18 mg, 0.20 mmol) in DCM (0.5 mL) slowly. The mixture was stirred at −50° C. for 0.5 h. The reaction mixture was quenched with water (5 mL) and extracted with DCM (5 mL) twice. The combined organic layers were dried over Na2SO4, filtered and concentrated in vacuo which was purified by prep-HPLC to afford 1-((2R,4S)-4-(4-amino-3-((1-ethyl-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (57.6 mg, 57%) as a yellow solid. 1H NMR (400 MHz, CDCl3): 8.56 (s, 1H), 7.94-7.65 (m, 3H), 7.47-7.27 (m, 2H), 6.75-6.72 (m, 1H), 6.57-6.41 (m, 2H), 6.45-6.40 (m, 2H), 5.76-5.70 (m, 1H), 5.52-5.30 (m, 1H), 4.66-4.50 (m, 1H), 4.22-4.10 (m, 2H), 3.93-3.87 (m, 1H), 3.02-3.43 (m, 1H), 3.40 (s, 3H), 3.02-2.71 (m, 1H), 2.64 (s, 3H), 2.54-2.40 (m, 1H), 1.44 (t, J=7.2 Hz, 3H). [M+H] Calcd.: 484.2; Found, 484.2.

### Example 22: 1-((2R,4S)-4-(4-amino-3-((2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

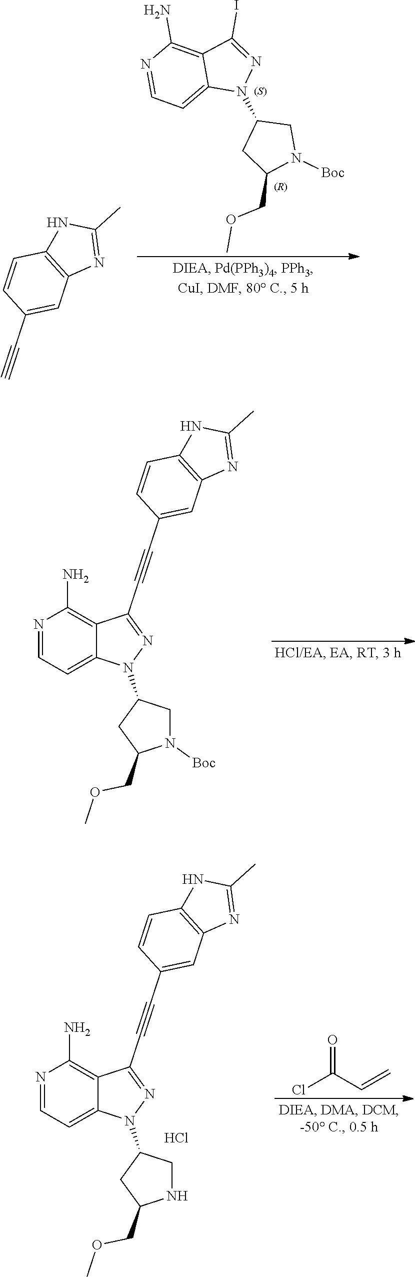

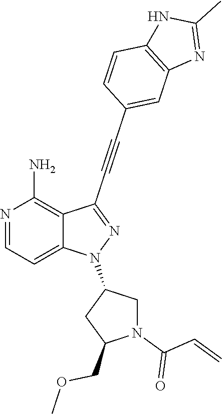 [SMILES: C=CC(=O)N1C[C@@H](n2nc(C#Cc3ccc4[nH]c(C)nc4c3)c3c(N)nccc32)C[C@@H]1COC]

### Step 1: (2R,4S)-tert-butyl 4-(4-amino-3-((2-methyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.40 mmol), 5-ethynyl-2-methyl-1H-benzo[d]imidazole (70 mg, 0.44 mmol), CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 5 h. After cooling down to room temperature, the reaction mixture was diluted with water (20 mL) and extracted with DCM (30 mL) for three times, the combined organic layers were dried over Na2SO4, filtered and concentrated in vacuo to afford (2R,4S)-tert-butyl 4-(4-amino-3-((2-methyl-1H-benzo[d]imidazol-5-yl)ethynyl)-1H-pyrazolo[4,3-c]pyridin-1-yl)-2-(methoxymethyl)pyrrolidine-1-carboxylate (180 mg, 90%) as a yellow solid. [M+H] Calcd.: 502.2; Found, 502.2.

### Step 2: 1-((3S,5R)-5-(methoxymethyl)pyrrolidin-3-yl)-3-((2-methyl-1H-benzo[d]imidazol-5-yl)ethynyl)-1H-pyrazolo[4,3-c]pyridin-4-amine hydrochloride

o a solution of (2R,4S)-tert-butyl 4-(4-amino-3-((2-methyl-1H-benzo[d]imidazol-5-yl)ethynyl)-1H-pyrazolo[4,3-c]pyridin-1-yl)-2-(methoxymethyl)pyrrolidine-1-carboxylate (180 mg, 0.36 mmol) in EA (5 mL) was added HCl/EA (15 mL) at room temperature. The reaction mixture was stirred at room temperature for 3 h. The reaction mixture was concentrated under vacuum to give 1-((3S,5R)-5-(methoxymethyl)pyrrolidin-3-yl)-3-((2-methyl-1H-benzo[d]imidazol-5-yl)ethynyl)-1H-pyrazolo[4,3-c]pyridin-4-amine hydrochloride (150 mg, 95%) as a yellow solid. [M+H] Calcd.: 402.2; Found, 402.2.

### Step 3: 1-((2R,4S)-4-(4-amino-3-((2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of 1-((3S,5R)-5-(methoxymethyl)pyrrolidin-3-yl)-3-((2-methyl-1H-benzo[d]imidazol-5-yl)ethynyl)-1H-pyrazolo[4,3-c]pyridin-4-amine hydrochloride (150 mg, 0.34 mmol), DIEA (132 mg, 1.02 mmol) in DCM (2 mL) and DMA (2 mL) at −50° C. under nitrogen atmosphere was added acryloyl chloride (31 mg, 0.34 mmol) in DCM (0.5 mL) slowly. The mixture was stirred at −50° C. for 0.5 h. The reaction mixture was quenched with water (5 mL) and extracted with DCM (5 mL) twice. The combined organic layers were dried over Na2SO4, filtered and concentrated in vacuo which was purified by prep-HPLC to afford 1-((2R,4S)-4-(4-amino-3-((2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (11 mg, 7%) as a yellow solid. 1H NMR (400 MHz, CDCl3): 8.50-8.00 (m, 1H), 7.62 (s, 1H), 7.51-7.44 (m, 2H), 7.29-7.25 (m, 1H), 6.69-6.65 (m, 1H), 6.53-6.34 (m, 2H), 5.72-5.64 (m, 1H), 5.42-5.25 (m, 1H), 4.60-4.43 (m, 1H), 4.10-4.03 (m, 2H), 3.84 (dd, J=2.4, 9.6 Hz, 1H), 3.48-3.40 (m, 1H), 3.33 (s, 3H), 2.86-2.60 (m, 4H), 2.53-2.34 (m, 1H). [M+H] Calcd.: 456.2; Found, 456.2.

### Example 23: 1-((2R,4S)-4-(4-amino-3-((1-ethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

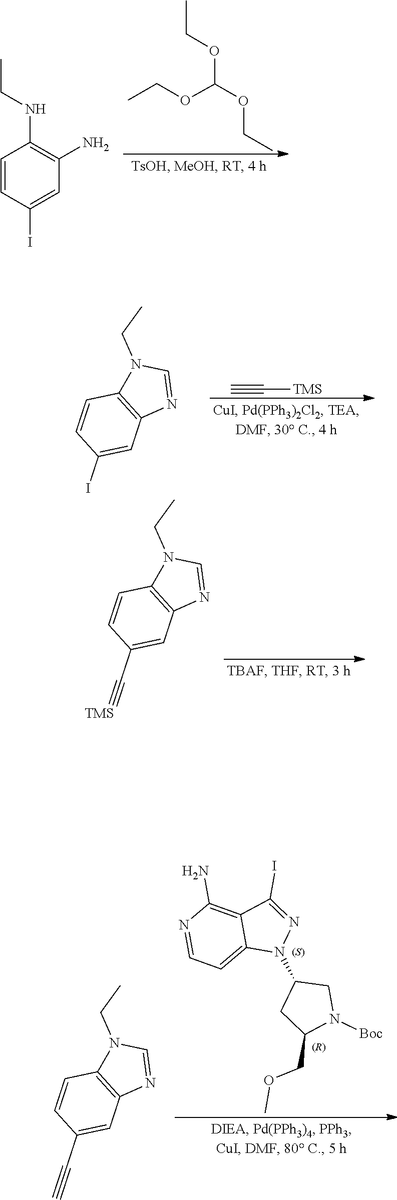

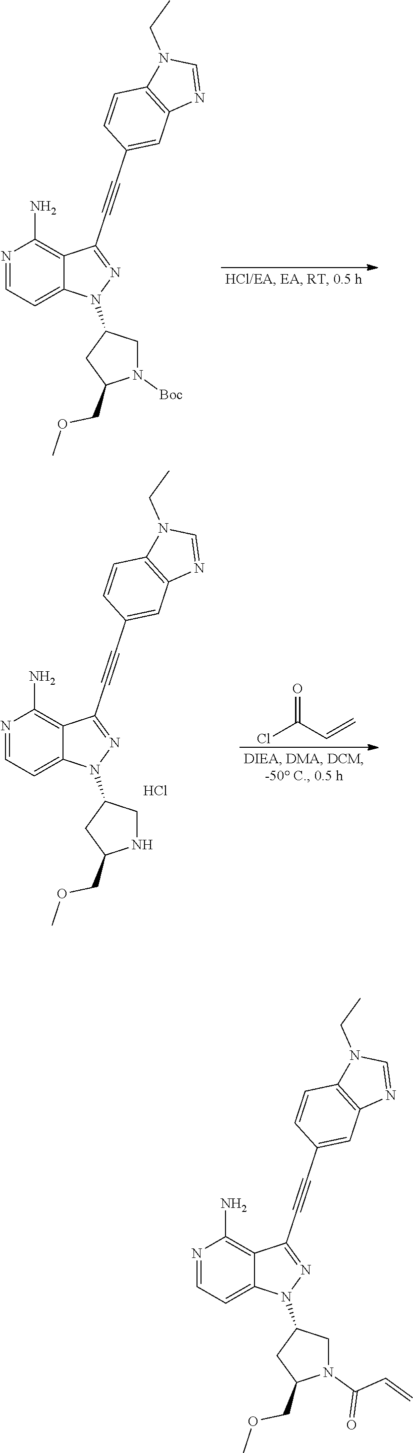

### Step 1: 1-ethyl-5-iodo-1H-benzo[d]imidazole

A mixture of N1-ethyl-4-iodobenzene-1,2-diamine (2.2 g, 8.40 mmol) and TsOH (145 mg, 0.84 mmol) in triethoxymethane (15 mL) and MeOH (15 mL) was stirred at room temperature for 4 h. Then the mixture was concentrated to give a residue which was dissolved in sat.NaHCO3 (20 mL) aq and extracted with EA (15 mL) for three times, The combined organic layers were washed with brine (20 mL), dried over Na2SO4, filtered and concentrated to give a residue, which was purified by flash (PE/EA=5/1) to give 1-ethyl-5-iodo-1H-benzo[d]imidazole (1.90 g, 83%) as a brown solid. [M+H] Calcd.: 273.0; Found, 273.0.

### Step 2: 1-ethyl-5-((trimethylsilyl)ethynyl)-1H-benzo[d]imidazole

To a mixture of 1-ethyl-5-iodo-1H-benzo[d]imidazole (2.75 g, 10.1 mmol), ethynyltrimethylsilane (1.98 g, 20.2 mmol), CuI (290 mg, 1.52 mmol) in DMF (25 mL) was added Pd(PPh3)2Cl2 (358 mg, 0.55 mmol), and TEA (1.54 g, 15.2 mmol) under nitrogen atmosphere and stirred at 30° C. for 4 h. The reaction mixture was diluted with water (50 mL) and extracted with EA (60 mL) for three times, the organic layers were dried over Na2SO4, filtered and concentrated in vacuo to give a residue, which was purified by flash (DCM/MeOH=50/1) to afford 1-ethyl-5-((trimethylsilyl)ethynyl)-1H-benzo[d]imidazole (2.30 g, 95%) as a brown solid. [M+H] Calcd.: 243.1; Found, 243.1.

### Step 3: 1-ethyl-5-ethynyl-1H-benzo[d]imidazole

To a solution of 1-ethyl-5-((trimethylsilyl)ethynyl)-1H-benzo[d]imidazole (2.3 g, 9.5 mmol) in THF (20 mL) was added 1 M TBAF in THF (9.5 mL, 9.5 mmol). Then the mixture was stirred for 3 h at rt. The reaction mixture was quenched with water (40 mL) and extracted with EA (30 mL) for three times. The combined organic layers were washed with brine (20 mL), dried over Na2SO4, filtered and concentrated in vacuo to give a residue, which was purified by flash (DCM/MeOH=50/1) to afford 1-ethyl-5-ethynyl-1H-benzo[d]imidazole (1.35 g, 96%) as a red solid. [M+H] Calcd.: 171.1; Found, 171.1.

### Step 4: (2R,4S)-tert-butyl 4-(4-amino-3-((1-ethyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.40 mmol), 1-ethyl-5-ethynyl-1H-benzo[d]imidazole (82 mg, 0.48 mmol), CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 5 h. After cooling down to room temperature, the reaction mixture was diluted with water (20 mL) and extracted with DCM (30 mL) for three times, the combined organic layers were dried over Na2SO4, filtered and concentrated in vacuo. The residue was purified by flash (DCM/MeOH=50/1) to afford ((2R,4S)-tert-butyl 4-(4-amino-3-((1-ethyl-1H-benzo[d]imidazol-5-yl)ethynyl)-1H-pyrazolo[4,3-c]pyridin-1-yl)-2-(methoxymethyl)pyrrolidine-1-carboxylate (190 mg, 93%) as a yellow solid. [M+H] Calcd.: 516.3; Found, 516.3.

### Step 5: 3-((1-ethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine hydrochloride

To a solution of ((2R,4S)-tert-butyl 4-(4-amino-3-((1-ethyl-1H-benzo[d]imidazol-5-yl)ethynyl)-1H-pyrazolo[4,3-c]pyridin-1-yl)-2-(methoxymethyl)pyrrolidine-1-carboxylate (190 mg, 0.37 mmol) in EA (5 mL) was added HCl/EA (15 mL) at room temperature. The reaction mixture was stirred at room temperature for 0.5 h. The reaction mixture was concentrated under vacuum to give 3-((1-ethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine hydrochloride (160 mg, 96%) as a yellow solid. [M+H] Calcd.: 416.2; Found, 416.2.

### Step 6: 1-((2R,4S)-4-(4-amino-3-((1-ethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of 3-((1-ethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine hydrochloride (160 mg, 0.35 mmol), DIEA (136 mg, 1.05 mmol) in DCM (2 mL) and DMA (2 mL) at −50° C. under nitrogen atmosphere was added acryloyl chloride (33 mg, 0.36 mmol) in DCM (0.5 mL) slowly. The mixture was stirred at −50° C. for 0.5 h. The reaction mixture was quenched with water (5 mL) and extracted with DCM (5 mL) twice. The combined organic layers were dried over Na2SO4, filtered and concentrated in vacuo which was purified by prep-HPLC to afford 1-((2R,4S)-4-(4-amino-3-((1-ethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one formate (75.2 mg, 46%) as a yellow solid. 1H NMR (400 MHz, DMSO-d6): 8.36 (s, 1H), 8.14 (s, 0.4H), 8.01 (s, 1H), 7.80 (d, J=8.0 Hz, 1H), 7.72 (d, J=8.4 Hz, 1H), 7.54 (dd, J=0.8, 8.0 Hz, 1H), 6.96 (d, J=6.0 Hz, 1H), 6.79-6.52 (m, 1H), 6.40 (br s, 2H), 6.20-6.13 (m, 1H), 5.72-5.65 (m, 1H), 5.50-5.46 (m, 1H), 4.48-4.47 (m, 1H), 4.32 (q, J=7.2 Hz, 2H), 4.09-3.81 (m, 2H), 3.61-3.48 (m, 2H), 3.33-3.22 (m, 3H), 2.67-2.57 (m, 1H), 2.42-2.37 (m, 1H), 1.43 (t, J=7.2 Hz, 3H). [M+H] Calcd.: 470.2; Found, 470.2.

### Example 25: 1-((2R,4S)-4-(4-amino-3-((1-cyclopropyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

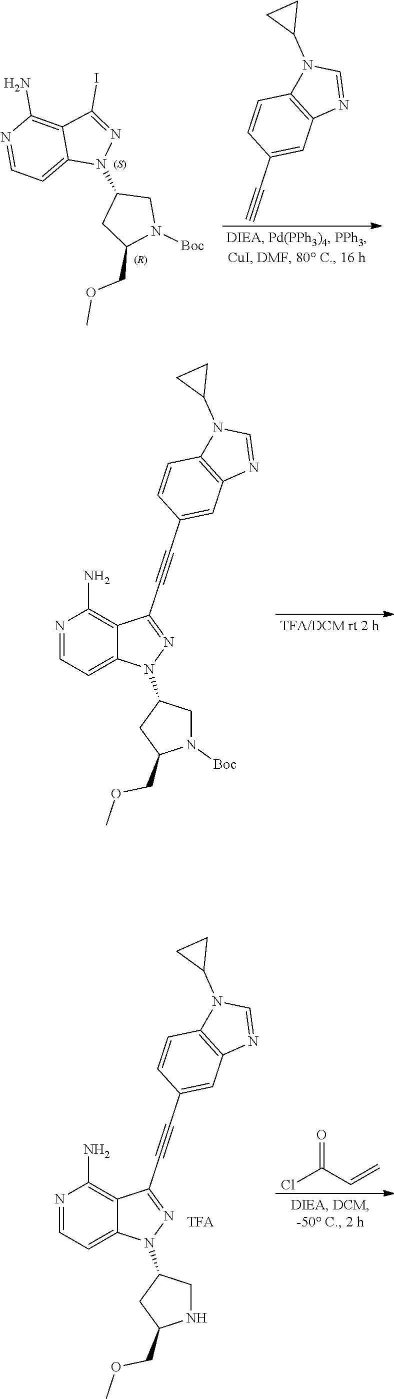

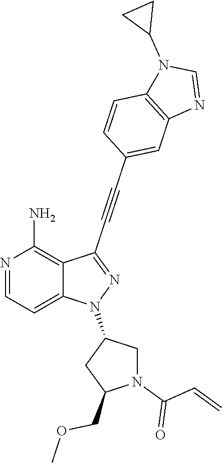 [SMILES: C=CC(=O)N1C[C@@H](n2nc(C#Cc3ccc4c(c3)ncn4C3CC3)c3c(N)nccc32)C[C@@H]1COC]

### Step 1: (2R,4S)-tert-butyl 4-(4-amino-3-((1-cyclopropyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.42 mmol), 1-cyclopropyl-5-ethynyl-1H-benzo[d]imidazole (91 mg, 0.51 mmol), CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 16 h. After cooling down to room temperature, the reaction mixture was diluted with water (20 mL) and extracted with DCM (30 mL) for three times, the combined organic layers were dried over Na2SO4, filtered and concentrated in vacuo. The residue was purified by flash (DCM/MeOH=20/1) to afford (2R,4S)-tert-butyl 4-(4-amino-3-((1-cyclopropyl-1H-benzo[d]imidazol-5-yl)ethynyl)-1H-pyrazolo[4,3-c]pyridin-1-yl)-2-(methoxymethyl)pyrrolidine-1-carboxylate (120 mg, 54%) as a brown solid. [M+H] Calcd.: 528.3; Found, 528.3.

### Step 2: 3-((1-cyclopropyl-1H-benzo[d]imidazol-5-yl)ethynyl)-1-((3S,5R)-5-(methoxymethyl)pyrrolidin-3-yl)-1H-pyrazolo[4,3-c]pyridin-4-amine TFA salt

To a solution of (2R,4S)-tert-butyl 4-(4-amino-3-((1-cyclopropyl-1H-benzo[d]imidazol-5-yl)ethynyl)-1H-pyrazolo[4,3-c]pyridin-1-yl)-2-(methoxymethyl)pyrrolidine-1-carboxylate (120 mg, 0.23 mmol) in DCM (2 mL) was added TFA (0.5 mL) at room temperature. The reaction mixture was stirred at room temperature for 2 h. The reaction mixture was concentrated under vacuum to give TFA salt 3-((1-cyclopropyl-1H-benzo[d]imidazol-5-yl)ethynyl)-1-((3S,5R)-5-(methoxymethyl)pyrrolidin-3-yl)-1H-pyrazolo[4,3-c]pyridin-4-amine (97 mg, crude) as a yellow solid. [M+H] Calcd.: 428.2; Found, 428.2.

### Step 3: 1-((2R,4S)-4-(4-amino-3-((1-cyclopropyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of 3-((1-cyclopropyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (97 mg, 0.23 mmol), DIEA (89 mg, 0.39 mmol) in DCM (15 mL) at −50° C. under nitrogen atmosphere was added acryloyl chloride (17 mg, 0.18 mmol) in DCM (0.5 mL) slowly. The mixture was stirred at −50° C. for 2 h. The reaction mixture was quenched with water (5 mL) and extracted with DCM (10 mL) for three times, the combined organic layers were dried over Na2SO4, filtered and concentrated in vacuo to give a residue, which was purified by prep-HPLC to afford 1-((2R,4S)-4-(4-amino-3-((1-cyclopropyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (10 mg, 9.1%) as a white solid. 1H NMR (400 MHz, DMSO-d6): 8.35 (s, 1H), 8.01 (s, 1H), 7.80 (d, J=6.4 Hz, 1H), 7.71 (d, J=8.4 Hz, 1H), 7.58 (d, J=8.4 Hz, 1H), 6.96 (d, J=6.0 Hz, 1H), 6.76-6.54 (m, 1H), 6.38 (br s, 2H), 6.18-6.14 (m, 1H), 5.70-5.66 (m, 1H), 5.54-5.42 (m, 1H), 4.59-4.48 (m, 1H), 4.07-3.81 (m, 2H), 3.57-3.48 (m, 5H), 2.66-2.50 (m, 2H), 2.42-2.38 (m, 1H), 1.13-1.06 (m, 4H). [M+H] Calcd.: 481.8; Found, 481.8.

### Example 25: 1-((2R,4S)-4-(4-amino-3-((1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

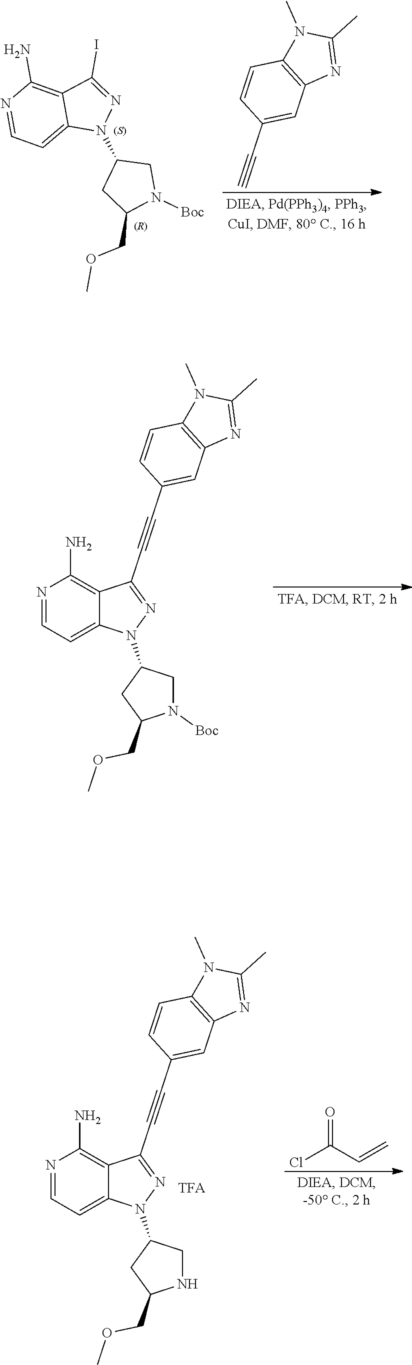

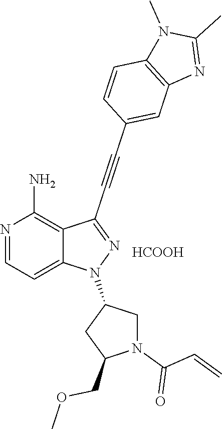

### Step 1: (2R,4S)-tert-butyl 4-(4-amino-3-((1,2-dimethyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.42 mmol), 5-ethynyl-1,2-dimethyl-1H-benzo[d]imidazole (86 mg, 0.51 mmol), CuI (11 mg, 0.06 mmol) in DMF (10 mL) was added Pd(PPh3)4 (23 mg, 0.02 mmol), PPh3 (6 mg, 0.02 mmol) and DIEA (155 mg, 1.20 mmol) under nitrogen atmosphere and stirred at 80° C. for 16 h. After cooling down to room temperature, the reaction mixture was diluted with water (20 mL) and extracted with DCM (30 mL) for three times, the combined organic layers were dried over Na2SO4, filtered and concentrated in vacuo. The residue was purified by flash (DCM/MeOH=20/1) to afford (2R,4S)-tert-butyl 4-(4-amino-3-((1,2-dimethyl-1H-benzo[d]imidazol-5-yl)ethynyl)-1H-pyrazolo[4,3-c]pyridin-1-yl)-2-(methoxymethyl)pyrrolidine-1-carboxylate (80 mg, 36%) as a brown solid. [M+H] Calcd.: 516.2; Found, 516.2.

### Step 2: 3-((1,2-dimethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate

To a solution of (2R,4S)-tert-butyl 4-(4-amino-3-((1,2-dimethyl-1H-benzo[d]imidazol-5-yl)ethynyl)-1H-pyrazolo[4,3-c]pyridin-1-yl)-2-(methoxymethyl)pyrrolidine-1-carboxylate (80 mg, 0.15 mmol) in DCM (2 mL) was added TFA (0.5 mL) at room temperature. The reaction mixture was stirred at room temperature for 2 h. The reaction mixture was concentrated under vacuum to give 3-((1,2-dimethyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (44 mg, crude) as a yellow solid. [M+H] Calcd.: 416.2; Found, 416.2.

### Step 3: 1-((2R,4S)-4-(4-amino-3-((1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one formate

To a solution of 3-((1-cyclopropyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (97 mg, 0.23 mmol), DIEA (89 mg, 0.39 mmol) in DCM (15 mL) at −50° C. under nitrogen atmosphere was added acryloyl chloride (17 mg, 0.18 mmol) in DCM (0.5 mL) slowly. The mixture was stirred at −50° C. for 2 h. The reaction mixture was quenched with water (5 mL) and extracted with DCM (10 mL) for three times, the combined organic layers were dried over Na₂SO₄, filtered and concentrated in vacuo to give a residue, which was purified by prep-HPLC to afford 1-((2R,4S)-4-(4-amino-3-((1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one formate (5.8 mg, 8%) as a white solid. ¹H NMR (400 MHz, DMSO-d₆): 8.17 (s, 1H), 7.85 (s, 1H), 7.79 (d, J=6.0 Hz, 1H), 7.58 (d, J=8.0 Hz, 1H), 7.58 (d, J=8.4 Hz, 1H), 7.47 (dd, J=0.8, 8.0 Hz, 1H), 6.95 (d, J=6.4 Hz, 1H), 6.79-6.53 (m, 1H), 6.38 (br s, 2H), 6.19-6.13 (m, 1H), 5.69-5.65 (m, 1H), 5.48-5.46 (m, 1H), 4.58-4.48 (m, 1H), 4.08-3.77 (m, 2H), 3.76 (s, 3H), 3.60-3.48 (m, 2H), 3.32 (s, 3H), 2.67-2.65 (m, 1H), 2.54 (s, 3H), 2.42-2.37 (m, 1H). [M+H] Calcd.: 470.9; Found, 470.9.

### Example 26: 1-((2R,4S)-4-(4-amino-3-((4,6-difluoro-1-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

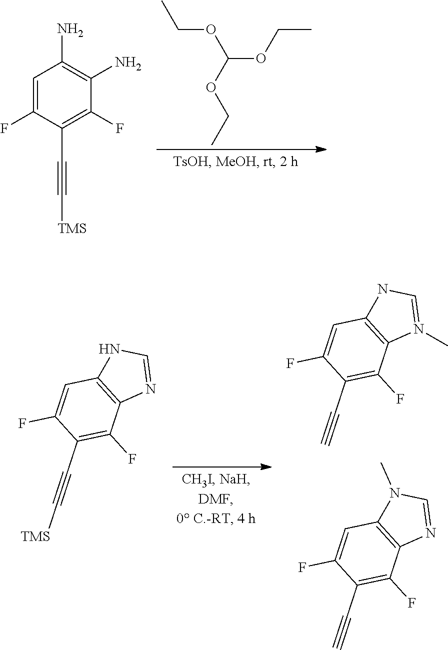

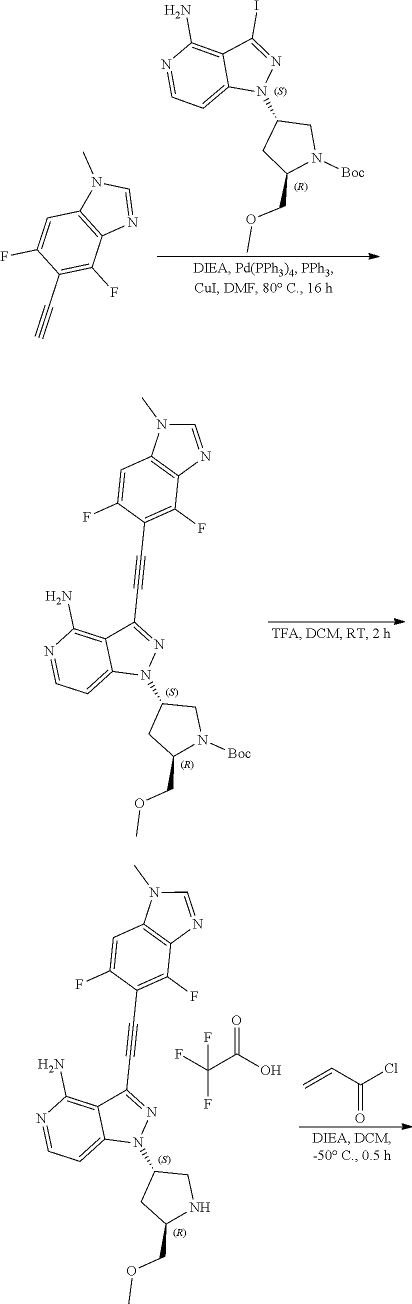

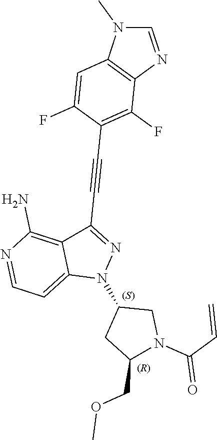

### Step 1: 4,6-difluoro-5-((trimethylsilyl)ethynyl)-1H-benzo[d]imidazole

To a solution of 3,5-difluoro-4-((trimethylsilyl)ethynyl)benzene-1,2-diamine (2.2 g, 9.17 mmol) in MeOH (15 mL) and triethoxymethane (15 mL) was added TsOH (157 mg, 0.92 mmol) at 0° C., the mixture was stirred at room temperature for 3 h. The reaction mixture was dissolved in EA (100 mL) and washed with water (30 mL). The organic layer was dried over Na2SO4, filtered and concentrated, the residue was purified by flash (PE/EA=1/1) to give 4,6-difluoro-5-((trimethylsilyl)ethynyl)-1H-benzo[d]imidazole (1.64 g, 71%) as a yellow solid. [M+H] Calcd.: 251.1; Found, 251.1.

### Step 2: 6-ethynyl-5,7-difluoro-1-methyl-1H-benzo[d]imidazole & 5-ethynyl-4,6-difluoro-1-methyl-1H-benzo[d]imidazole

To a solution of 4,6-difluoro-5-((trimethylsilyl)ethynyl)-1H-benzo[d]imidazole (1.64 g, 6.51 mmol) in DMF (35 mL) was added NaH (312 mg, 7.818 mmol, 60% wt.) at 0° C., the mixture was stirred at 0° C. for 20 min, then added dropwise CH3I (1.11 g, 7.81 mmol). The mixture was stirred at room temperature for 4 h. The mixture was diluted with water (10 mL) and extracted with EA (10 mL) for three times, the combined organic layers were dried over Na2SO4, filtered and concentrated in vacuo to give a residue, which was purified by flash (PE/EA=1/1) to afford a mixture of 6-ethynyl-5,7-difluoro-1-methyl-1H-benzo[d]imidazole and 5-ethynyl-4,6-difluoro-1-methyl-1H-benzo[d]imidazole. The mixture was further purified by chiral-HPLC to give 6-ethynyl-5,7-difluoro-1-methyl-1H-benzo[d]imidazole (290 mg, 24%) and 5-ethynyl-4,6-difluoro-1-methyl-1H-benzo[d]imidazole (300 mg, 25%) as a yellow solid. [M+H] Calcd.: 193.1; Found, 193.1.

### Step 3: (2R,4S)-tert-butyl 4-(4-amino-3-((4,6-difluoro-1-methyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.42 mmol), 5-ethynyl-4,6-difluoro-1-methyl-1H-benzo[d]imidazole (97 mg, 0.51 mmol), CuI (9 mg, 0.05 mmol) in DMF (5 mL) was added Pd(PPh3)4 (243 mg, 0.21 mmol), PPh3 (5 mg, 0.02 mmol) and DIEA (223 mg, 1.51 mmol) under nitrogen atmosphere and stirred at 80° C. for 16 h. After cooling down to room temperature, the reaction mixture was diluted with water (20 mL) and extracted with DCM (20 mL) for three times, the combined organic layers were dried over Na2SO4, filtered and concentrated in vacuo which was purified by flash (DCM/MeOH=20/1) to afford (2R,4S)-tert-butyl 4-(4-amino-3-((4,6-difluoro-1-methyl-1H-benzo[d]imidazol-5-yl)ethynyl)-1H-pyrazolo[4,3-c]pyridin-1-yl)-2-(methoxymethyl)pyrrolidine-1-carboxylate (130 mg, 57%) as a yellow solid. [M+H] Calcd.: 538.2; Found, 538.2.

### Step 4: 3-((4,6-difluoro-1-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate

To a solution of (2R,4S)-tert-butyl 4-(4-amino-3-((4,6-difluoro-1-methyl-1H-benzo[d]imidazol-5-yl)ethynyl)-1H-pyrazolo[4,3-c]pyridin-1-yl)-2-(methoxymethyl)pyrrolidine-1-carboxylate (130 mg, 0.24 mmol) in DCM (2 mL) was added TFA (0.5 mL), the mixture was stirred at room temperature for 2 h. The reaction mixture was concentrated in vacuo to afford 3-((4,6-difluoro-1-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (105 mg, crude) as yellow oil. [M+H] Calcd.: 438.2; Found, 438.2.

### Step 5: 1-((2R,4S)-4-(4-amino-3-((4,6-difluoro-1-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of 3-((4,6-difluoro-1-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (125 mg, 0.24 mmol), DIEA (93 mg, 0.72 mmol) in DCM (15 mL) at −50° C. under nitrogen atmosphere was added acryloyl chloride (19 mg, 0.24 mmol) in DCM (0.5 mL) slowly. The mixture was stirred at −50° C. for 0.5 h. The reaction mixture was quenched with water (15 mL) and extracted with DCM (10 mL) twice. The combined organic layers were dried over Na2SO4, filtered and concentrated in vacuo to give a residue, which was purified by prep-HPLC to afford 1-((2R,4S)-4-(4-amino-3-((4,6-difluoro-1-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (56 mg, 47%) as a white solid. 1H NMR (400 MHz, DMSO-d6): 8.38 (s, 1H), 7.82 (d, J=6.4 Hz, 1H), 7.67 (d, J=8.8 Hz, 1H), 6.98 (d, J=6.4 Hz, 1H), 6.76-6.50 (m, 3H), 6.19-6.13 (m, 1H), 5.72-5.65 (m, 1H), 5.52-5.47 (m, 1H), 4.60-4.46 (m, 1H), 4.11-3.89 (m, 1H), 3.87 (s, 3H), 3.83-3.81 (m, 1H), 3.63-3.47 (m, 2H), 3.30 (s, 3H), 2.71-2.55 (m, 1H), 2.43-2.38 (m, 1H). [M+H] Calcd.: 491.8; Found, 491.8.

### Example 27: 1-((2R,4S)-4-(4-amino-3-((1-methyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one

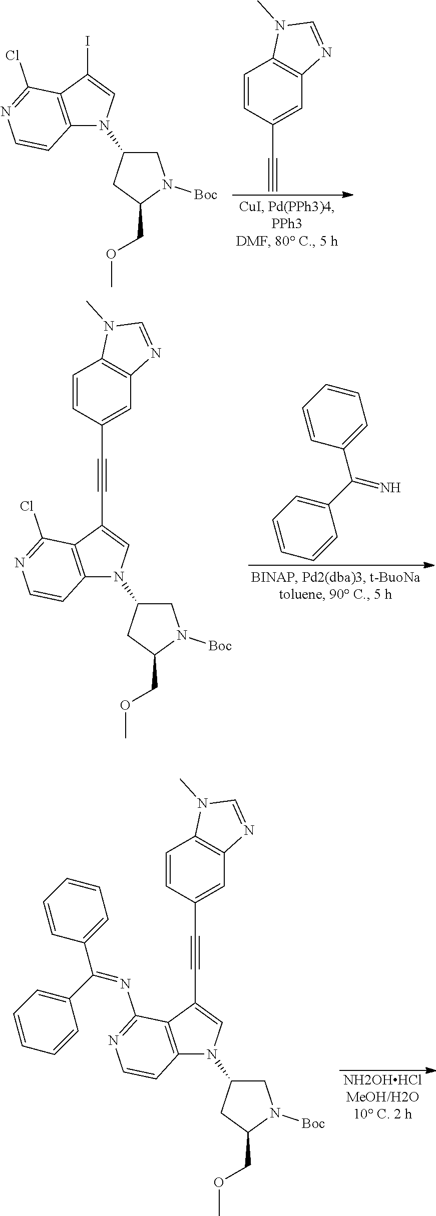

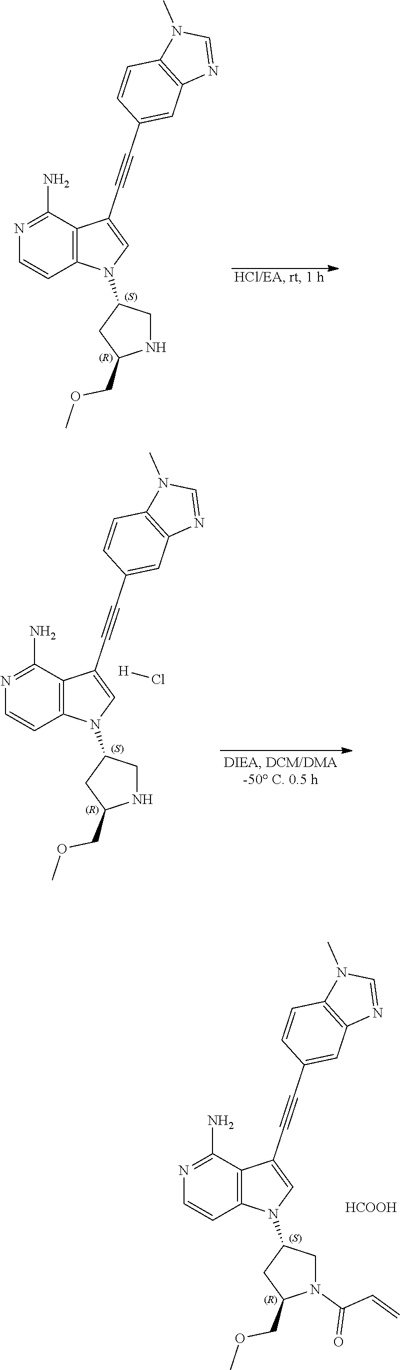

### Step 1: (2R,4S)-tert-butyl 4-(4-chloro-3-((1-methyl-1H-benzo[d]imidazol-5-yl)ethynyl)-1H-pyrrolo[3,2-c]pyridin-1-yl)-2-(methoxymethyl)pyrrolidine-1-carboxylate

To a mixture of (2R,4S)-tert-butyl 4-(4-chloro-3-iodo-1H-pyrrolo[3,2-c]pyridin-1-yl)-2-(methoxymethyl)pyrrolidine-1-carboxylate (600 mg, 1.2 mmol), 5-ethynyl-1-methyl-1H-benzo[d]imidazole (267 mg, 1.7 mmol), CuI (15 mg, 0.07 mmol) in DMF (20 mL) was added Pd(PPh3)4 (71 mg, 0.06 mmol), PPh3 (16 mg, 0.06 mmol) and DIEA (473 mg, 3.66 mmol) under nitrogen atmosphere and stirred at 80° C. for 5 h. After cooling down to room temperature, the reaction mixture was diluted with water (60 mL) and extracted with EA (30 mL) for three times, the combined organic layers were dried over Na2SO4, filtered and concentrated in vacuo. The residue was purified by prep-HPLC to afford (2R,4S)-tert-butyl 4-(4-chloro-3-((1-methyl-1H-benzo[d]imidazol-5-yl)ethynyl)-1H-pyrrolo[3,2-c]pyridin-1-yl)-2-(methoxymethyl)pyrrolidine-1-carboxylate (60 mg, 9%) as a yellow solid. [M+H] Calcd.: 520.2; Found, 520.2.

### Step 2: (2R,4S)-tert-butyl 4-(4-((diphenylmethylene)amino)-3-((1-methyl-1H-benzo[d]imidazol-5-yl)ethynyl)-1H-pyrrolo[3,2-c]pyridin-1-yl)-2-(methoxymethyl)pyrrolidine-1-carboxylate

A solution of (2R,4S)-tert-butyl 4-(4-chloro-3-((1-methyl-1H-benzo[d]imidazol-5-yl)ethynyl)-1H-pyrrolo[3,2-c]pyridin-1-yl)-2-(methoxymethyl)pyrrolidine-1-carboxylate (600 mg, 1.15 mmol), diphenylmethanimine (420 mg, 2.32 mmol), Pd2(dba)3 (212 mg, 0.23 mmol), BINAP (144 mg, 0.23 mmol) and t-BuONa (333 mg, 3.45 mmol) in toluene (20 mL) was stirred at 90° C. for 5 h under nitrogen atmosphere. After cooling down to room temperature, the reaction mixture was diluted with water (20 mL) and extracted with EA (30 mL) for three times, the organic layers were dried over Na2SO4, filtered and concentrated in vacuo. The residue was purified by flash (DCM/MeOH=20/1) to afford (2R,4S)-tert-butyl 4-(4-((diphenylmethylene)amino)-3-((1-methyl-1H-benzo[d]imidazol-5-yl)ethynyl)-1H-pyrrolo[3,2-c]pyridin-1-yl)-2-(methoxymethyl)pyrrolidine-1-carboxylate (330 mg, 43%) as a yellow solid. [M+H] Calcd.: 665.3; Found, 665.3.

### Step 3: (2R,4S)-tert-butyl 4-(4-amino-3-((1-methyl-1H-benzo[d]imidazol-5-yl)ethynyl)-1H-pyrrolo[3,2-c]pyridin-1-yl)-2-(methoxymethyl)pyrrolidine-1-carboxylate

To a solution of (2R,4S)-tert-butyl 4-(4-((diphenylmethylene)amino)-3-((1-methyl-1H-benzo[d]imidazol-5-yl)ethynyl)-1H-pyrrolo[3,2-c]pyridin-1-yl)-2-(methoxymethyl)pyrrolidine-1-carboxylate (300 mg, 0.45 mmol) in MeOH (4 mL) and H2O (4 mL) was added hydroxylamine hydrochloride (790 mg, 11.3 mmol) and NaHCO₃ (949 mg, 11.3 mmol) and stirred at 10° C. for 2 h. The reaction mixture was diluted with water (20 mL) and extracted with EA (10 mL) for three times, the organic layers were dried over Na2SO4, filtered and concentrated in vacuo. The residue was purified by prep-HPLC to afford (2R,4S)-tert-butyl 4-(4-amino-3-((1-methyl-1H-benzo[d]imidazol-5-yl)ethynyl)-1H-pyrrolo[3,2-c]pyridin-1-yl)-2-(methoxymethyl)pyrrolidine-1-carboxylate (225 mg, 100%) as a yellow solid. [M+H] Calcd.: 501.2 Found, 501.2.

### Step 4: 1-((3S,5R)-5-(methoxymethyl)pyrrolidin-3-yl)-3-((1-methyl-1H-benzo[d]imidazol-5-yl)ethynyl)-1H-pyrrolo[3,2-c]pyridin-4-amine hydrochloride

To a solution of (2R,4S)-tert-butyl 4-(4-amino-3-((1-methyl-1H-benzo[d]imidazol-5-yl)ethynyl)-1H-pyrrolo[3,2-c]pyridin-1-yl)-2-(methoxymethyl)pyrrolidine-1-carboxylate (225 mg, 0.45 mmol) in THF (10 mL) was added EA/HCl (10 mL) at room temperature. The reaction mixture was stirred at room temperature for 1 h. The reaction mixture was concentrated under vacuum to give 1-((3S,5R)-5-(methoxymethyl)pyrrolidin-3-yl)-3-((1-methyl-1H-benzo[d]imidazol-5-yl)ethynyl)-1H-pyrrolo[3,2-c]pyridin-4-amine hydrochloride (210 mg, crude) as a yellow solid. [M+H] Calcd.: 401.2; Found, 401.2.

### Step 5: 1-((2R,4S)-4-(4-amino-3-((1-methyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate

To a solution of 1-((3S,5R)-5-(methoxymethyl)pyrrolidin-3-yl)-3-((1-methyl-1H-benzo[d]imidazol-5-yl)ethynyl)-1H-pyrrolo[3,2-c]pyridin-4-amine hydrochloride (210 mg, 0.48 mmol), DIEA (187 mg, 1.44 mmol) in DCM (2 mL) and DMA (2 mL) at −50° C. under nitrogen atmosphere was added acryloyl chloride (43 mg, 0.48 mmol) in DCM (0.5 mL) slowly. The mixture was stirred at −50° C. for 0.5 h. The reaction mixture was quenched with water (5 mL) and extracted with DCM (10 mL) for three times, the combined organic were dried over Na2SO4, filtered and concentrated in vacuo to give a residue, which was purified by prep-HPLC to afford 1-((2R,4S)-4-(4-amino-3-((1-methyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate (58.4 mg, 60%) as a white solid. 1H NMR (400 MHz, DMSO-d6): 8.27 (s, 1H), 8.14 (s, 1H), 7.85-7.77 (m, 2H), 7.71-7.62 (m, 2H), 7.44 (dd, J=1.2, 9.2 Hz, 1H), 6.97 (d, J=6.8 Hz, 1H), 6.76-6.53 (m, 1H), 6.29-6.11 (m, 3H), 5.74-5.56 (m, 1H), 5.31-5.28 (m, 1H), 4.46-4.45 (m, 1H), 4.10-4.07 (m, 1H), 3.86 (s, 3H), 3.78-3.74 (m, 1H), 3.62-3.49 (m, 2H), 3.31 (s, 3H), 2.67-2.55 (m, 1H), 243-2.40 (m, 1H). [M+H] Calcd.: 454.9; Found, 454.9.

### Example 28: 1-((2R,4S)-4-(4-amino-3-((1,2-dimethyl-1H-benzo[d]imidazol-6-yl)ethynyl)-1H-pyrazolo[4,3-c]pyridin-1-yl)-2-(methoxymethyl)pyrrolidin-1-yl)prop-2-en-1-one

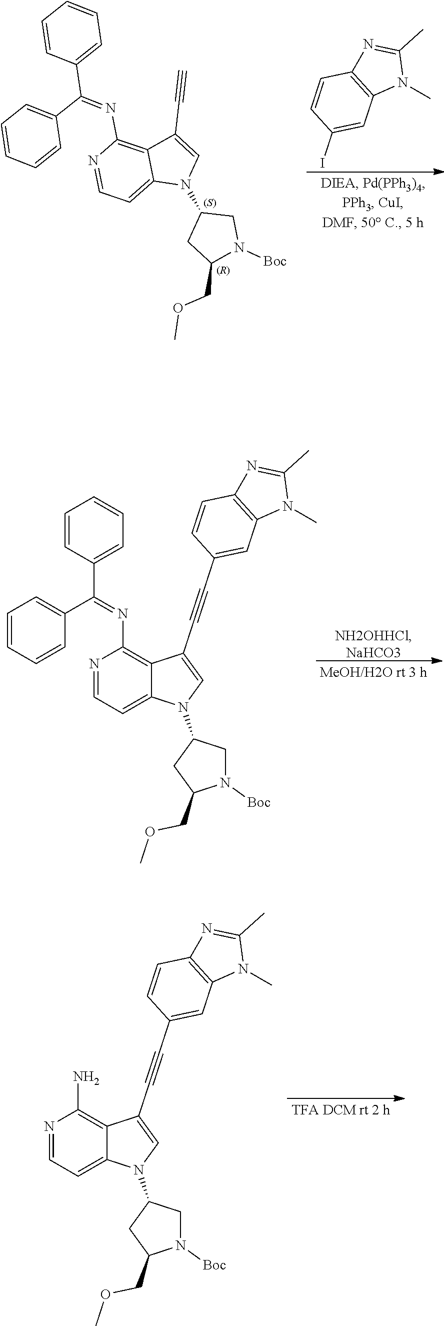

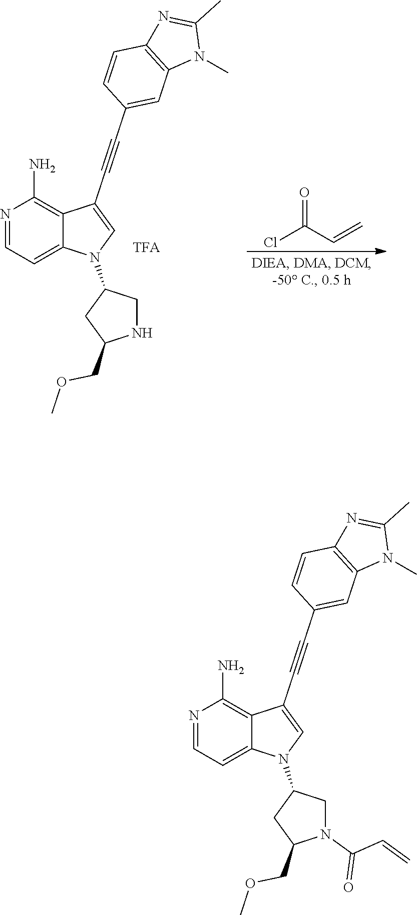

### Step 1: (2R,4S)-tert-butyl 4-(3-((1,2-dimethyl-1H-benzo[d]imidazol-6-yl)ethynyl)-4-((diphenylmethylene)amino)-1H-pyrrolo[3,2-c]pyridin-1-yl)-2-(methoxymethyl)pyrrolidine-1-carboxylate

To a mixture of (2R,4S)-tert-butyl 4-(4-((diphenylmethylene)amino)-3-((trimethylsilyl)ethynyl)-1H-pyrrolo[3,2-c]pyridin-1-yl)-2-(methoxymethyl)pyrrolidine-1-carboxylate (200 mg, 0.37 mmol), 6-iodo-1,2-dimethyl-1H-benzo[d]imidazole (122 mg, 0.45 mmol), CuI (21 mg, 0.12 mmol) in DMF (10 mL) was added Pd(PPh3)4 (86 mg, 0.07 mmol), PPh3 (97 mg, 0.04 mmol) and DIEA (148 mg, 1.12 mmol) under nitrogen atmosphere and stirred at 50° C. for 5 h. The reaction mixture was diluted with water (20 mL) and extracted with EA (30 mL) for three times, the combined organic layers were dried over Na2SO4, filtered and concentrated in vacuo. The residue was purified by flash (DCM/MeOH=20/1) to afford (2R,4S)-tert-butyl 4-(3-((1,2-dimethyl-1H-benzo[d]imidazol-6-yl)ethynyl)-4-((diphenylmethylene)amino)-1H-pyrrolo[3,2-c]pyridin-1-yl)-2-(methoxymethyl)pyrrolidine-1-carboxylate (100 mg, 39%) as a brown solid. [M+H] Calcd.: 679.3; Found, 679.3.

### Step 2: (2R,4S)-tert-butyl 4-(4-amino-3-((1,2-dimethyl-1H-benzo[d]imidazol-6-yl)ethynyl)-1H-pyrrolo[3,2-c]pyridin-1-yl)-2-(methoxymethyl)pyrrolidine-1-carboxylate

To a solution of (2R,4S)-tert-butyl 4-(3-((1,2-dimethyl-1H-benzo[d]imidazol-6-yl)ethynyl)-4-((diphenylmethylene)amino)-1H-pyrrolo[3,2-c]pyridin-1-yl)-2-(methoxymethyl)pyrrolidine-1-carboxylate (100 mg, 0.14 mmol) in MeOH (5 mL) and H2O (3 mL) was added hydroxylamine hydrochloride (258 mg, 0.68 mmol) and NaHCO₃ (309 mg, 3.68 mmol) and stirred at rt for 3 h. The reaction mixture was diluted with water (20 mL) and extracted with EA (10 mL) for three times, the combined organic layers were dried over Na2SO4, filtered and concentrated in vacuo. The residue was purified by flash (DCM/MeOH=20/1) to afford (2R,4S)-tert-butyl 4-(4-amino-3-((1,2-dimethyl-1H-benzo[d]imidazol-6-yl)ethynyl)-1H-pyrrolo[3,2-c]pyridin-1-yl)-2-(methoxymethyl)pyrrolidine-1-carboxylate (50 mg, 46%) as a brown solid. [M+H] Calcd.: 515.2 Found, 515.2.

### Step 3: 3-((1,2-dimethyl-1H-benzo[d]imidazol-6-yl)ethynyl)-1-((3S,5R)-5-(methoxymethyl)pyrrolidin-3-yl)-1H-pyrrolo[3,2-c]pyridin-4-amine 2,2,2-trifluoroacetate

To a solution of (2R,4S)-tert-butyl 4-(4-amino-3-((1,2-dimethyl-1H-benzo[d]imidazol-6-yl)ethynyl)-1H-pyrrolo[3,2-c]pyridin-1-yl)-2-(methoxymethyl)pyrrolidine-1-carboxylate (50 mg, 0.097 mmol) in DCM (0.5 mL) was added TFA (0.2 mL) at room temperature. The reaction mixture was stirred at room temperature for 2 h. The reaction mixture was concentrated under vacuum to give 3-((1,2-dimethyl-1H-benzo[d]imidazol-6-yl)ethynyl)-1-((3S,5R)-5-(methoxymethyl)pyrrolidin-3-yl)-1H-pyrrolo[3,2-c]pyridin-4-amine 2,2,2-trifluoroacetate (40 mg, crude) as a yellow solid. [M+H] Calcd.: 415.2; Found, 415.2.

### Step 4: 1-((2R,4S)-4-(4-amino-3-((1,2-dimethyl-1H-benzo[d]imidazol-6-yl)ethynyl)-1H-pyrrolo[3,2-c]pyridin-1-yl)-2-(methoxymethyl)pyrrolidin-1-yl)prop-2-en-1-one

To a solution of 3-((1,2-dimethyl-1H-benzo[d]imidazol-6-yl)ethynyl)-1-((3S,5R)-5-(methoxymethyl)pyrrolidin-3-yl)-1H-pyrrolo[3,2-c]pyridin-4-amine 2,2,2-trifluoroacetate (40 mg, 0.097 mmol), DIEA (37 mg, 0.29 mmol) in DCM (15 mL) and DMA (0.5 mL) at −50° C. under nitrogen atmosphere was added acryloyl chloride (7 mg, 0.078 mmol) in DCM (0.5 mL) slowly. The mixture was stirred at −50° C. for 0.5 h. The reaction mixture was quenched with water (5 mL) and extracted with DCM (10 mL) for three times, the combined organic layers were dried over Na2SO4, filtered and concentrated in vacuo to give a residue, which was purified by prep-HPLC to afford 1-((2R,4S)-4-(4-amino-3-((1,2-dimethyl-1H-benzo[d]imidazol-6-yl)ethynyl)-1H-pyrrolo[3,2-c]pyridin-1-yl)-2-(methoxymethyl)pyrrolidin-1-yl)prop-2-en-1-one (10.6 mg, 23%) as a white solid. 1H NMR (400 MHz, DMSO-d6): 7.71-7.67 (m, 3H), 7.53 (d, J=8.4 Hz, 1H), 7.28 (d, J=8.0 Hz, 1H), 6.90-6.88 (m, 1H), 6.82-6.53 (m, 1H), 6.22-6.15 (m, 1H), 5.99 (br s, 2H), 5.74-5.67 (m, 1H), 5.31-5.21 (m, 1H), 4.60-4.45 (m, 1H), 4.09-3.75 (m, 5H), 3.60-3.48 (m, 2H), 3.32 (s, 3H), 2.60-2.55 (m, 1H), 2.54 (s, 3H), 2.40-2.35 (m, 1H). [M+H] Calcd.: 469.2; Found, 469.2.

### Example 29: 1-((2R,4S)-4-(4-amino-3-((1,2-dimethyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one

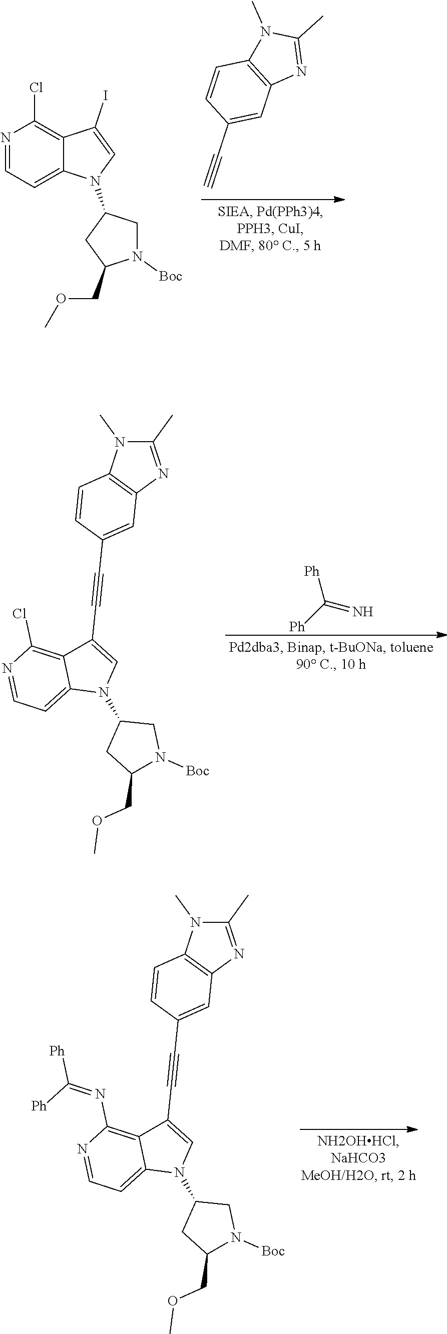 [SMILES: COC[C@H]1C[C@H](n2cc(I)c3c(Cl)nccc32)CN1C(=O)OC(C)(C)C ; C#Cc1ccc2c(c1)nc(C)n2C ; COC[C@H]1C[C@H](n2cc(C#Cc3ccc4c(c3)nc(C)n4C)c3c(Cl)nccc32)CN1C(=O)OC(C)(C)C ; N=C(c1ccccc1)c1ccccc1 ; COC[C@H]1C[C@H](n2cc(C#Cc3ccc4c(c3)nc(C)n4C)c3c(N=C(c4ccccc4)c4ccccc4)nccc32)CN1C(=O)OC(C)(C)C]

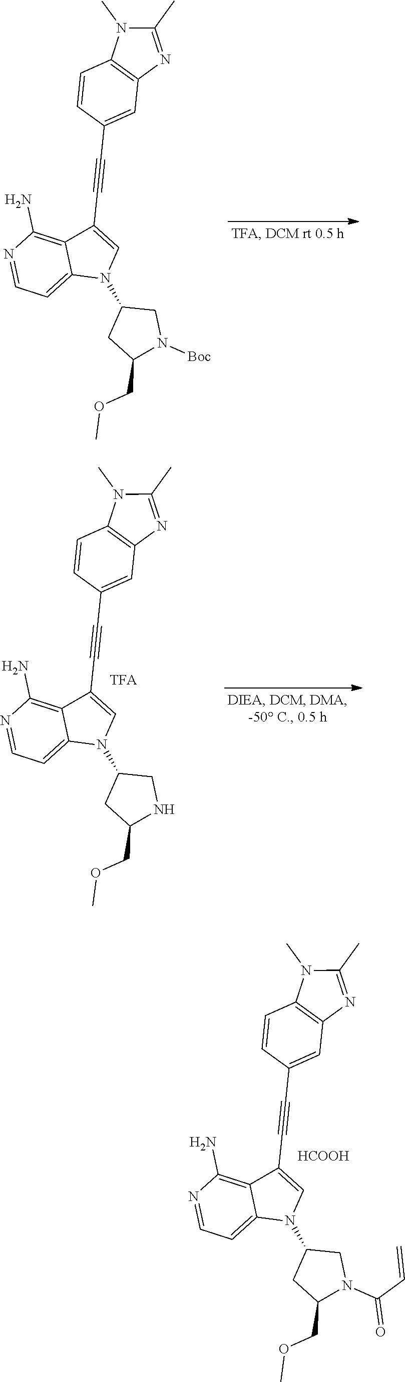

### Step 1: (2R,4S)-tert-butyl 4-(4-chloro-3-((1,2-dimethyl-1H-benzo[d]imidazol-5-yl)ethynyl)-1H-pyrrolo[3,2-c]pyridin-1-yl)-2-(methoxymethyl)pyrrolidine-1-carboxylate

To a mixture of (2R,4S)-tert-butyl 4-(4-chloro-3-iodo-1H-pyrrolo[3,2-c]pyridin-1-yl)-2-(methoxymethyl)pyrrolidine-1-carboxylate (736 mg, 1.5 mmol), 5-ethynyl-1,2-dimethyl-1H-benzo[d]imidazole (255 mg, 1.5 mmol), CuI (85 mg, 0.45 mmol) in DMF (30 mL) was added Pd(PPh3)4 (173 mg, 0.15 mmol), PPh3 (39 mg, 0.15 mmol) and DIEA (585 mg, 4.53 mmol) under nitrogen atmosphere and stirred at 80° C. for 5 h. After cooling down to room temperature, the reaction mixture was diluted with water (60 mL) and extracted with EA (30 mL) for three times, the combined organic layers were dried over Na2SO4, filtered and concentrated in vacuo. The residue was purified by flash (DCM/MeOH=20/1) to afford (2R,4S)-tert-butyl 4-(4-chloro-3-((1,2-dimethyl-1H-benzo[d]imidazol-5-yl)ethynyl)-1H-pyrrolo[3,2-c]pyridin-1-yl)-2-(methoxymethyl)pyrrolidine-1-carboxylate (370 mg, 39%) as a yellow solid. [M+H] Calcd.: 534.2; Found, 534.2.

### Step 2: (2R,4S)-tert-butyl 4-(3-((1,2-dimethyl-1H-benzo[d]imidazol-5-yl)ethynyl)-4-((diphenylmethylene)amino)-1H-pyrrolo[3,2-c]pyridin-1-yl)-2-(methoxymethyl)pyrrolidine-1-carboxylate

A solution of (2R,4S)-tert-butyl 4-(4-chloro-3-((1,2-dimethyl-1H-benzo[d]imidazol-5-yl)ethynyl)-1H-pyrrolo[3,2-c]pyridin-1-yl)-2-(methoxymethyl)pyrrolidine-1-carboxylate (370 mg, 0.7 mmol), diphenylmethanimine (253 mg, 1.4 mmol), Pd2(dba)3 (128 mg, 0.14 mmol), BINAP (87 mg, 0.14 mmol) and t-BuONa (202 mg, 2.1 mmol) in toluene (20 mL) was stirred at 90° C. for 10 h under nitrogen atmosphere. After cooling down to room temperature, the reaction mixture was diluted with water (20 mL) and extracted with EA (30 mL) for three times, the combined organic layers were dried over Na2SO4, filtered and concentrated in vacuo. The residue was purified by flash (DCM/MeOH=20/1) to afford (2R,4S)-tert-butyl 4-(3-((1,2-dimethyl-1H-benzo[d]imidazol-5-yl)ethynyl)-4-((diphenylmethylene)amino)-1H-pyrrolo[3,2-c]pyridin-1-yl)-2-(methoxymethyl)pyrrolidine-1-carboxylate (370 mg, 78%) as a yellow solid. [M+H] Calcd.: 679.3; Found, 679.3.

### Step 3: (2R,4S)-tert-butyl 4-(4-amino-3-((1,2-dimethyl-1H-benzo[d]imidazol-5-yl)ethynyl)-1H-pyrrolo[3,2-c]pyridin-1-yl)-2-(methoxymethyl)pyrrolidine-1-carboxylate

To a solution of (2R,4S)-tert-butyl 4-(3-((1,2-dimethyl-1H-benzo[d]imidazol-5-yl)ethynyl)-4-((diphenylmethylene)amino)-1H-pyrrolo[3,2-c]pyridin-1-yl)-2-(methoxymethyl)pyrrolidine-1-carboxylate (370 mg, 0.54 mmol) in MeOH (20 mL) and H2O (20 mL) was added hydroxylamine hydrochloride (945 mg, 13.5 mmol) and NaHCO₃ (1.10 g, 13.5 mmol) and stirred at rt for 2 h. The reaction mixture was diluted with water (20 mL) and extracted with EA (10 mL) for three times, the combined organic layers were dried over Na2SO4, filtered and concentrated in vacuo. The residue was purified by flash (DCM/MeOH=20/1) to afford (2R,4S)-tert-butyl 4-(4-amino-3-((1,2-dimethyl-1H-benzo[d]imidazol-5-yl)ethynyl)-1H-pyrrolo[3,2-c]pyridin-1-yl)-2-(methoxymethyl)pyrrolidine-1-carboxylate (110 mg, 40%) as a yellow solid. [M+H] Calcd.: 515.2 Found, 515.2.

### Step 4: 3-((1,2-dimethyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate

To a solution of (2R,4S)-tert-butyl 4-(4-amino-3-((1,2-dimethyl-1H-benzo[d]imidazol-5-yl)ethynyl)-1H-pyrrolo[3,2-c]pyridin-1-yl)-2-(methoxymethyl)pyrrolidine-1-carboxylate (110 mg, 0.21 mmol) in DCM (2 mL) was added TFA (1 mL) at room temperature. The reaction mixture was stirred at room temperature for 0.5 h. The reaction mixture was concentrated under vacuum to give 3-((1,2-dimethyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate (97 mg, crude) as a yellow solid. [M+H] Calcd.: 415.2; Found, 415.2.

### Step 5: 1-((2R,4S)-4-(4-amino-3-((1,2-dimethyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate

To a solution of 3-((1,2-dimethyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate (97 mg, 0.21 mmol), DIEA (82 mg, 0.63 mmol) in DCM (1 mL) and DMA (1 mL) at −50° C. under nitrogen atmosphere was added acryloyl chloride (20 mg, 0.21 mmol) in DCM (0.5 mL) slowly. The mixture was stirred at −50° C. for 0.5 h. The reaction mixture was quenched with water (5 mL) and extracted with DCM (10 mL) for three times, the combined organic layers were dried over Na2SO4, filtered and concentrated in vacuo which was purified by prep-HPLC to afford 1-((2R,4S)-4-(4-amino-3-((1,2-dimethyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate (58.4 mg, 60%) as a white solid. 1H NMR (400 MHz, DMSO-d6): 8.15 (s, 1H), 7.79 (d, J=8.8 Hz, 1H), 7.71-7.70 (m, 2H), 7.53 (d, J=8.4 Hz, 1H), 7.36 (dd, J=1.2, 8.4 Hz, 1H), 7.00 (d, J=6.4 Hz, 1H), 6.80-6.53 (m, 1H), 6.42-6.38 (m, 2H), 6.21-6.15 (m, 2H), 5.74-5.66 (m, 1H), 5.31-5.27 (m, 1H), 4.63-4.45 (m, 1H), 4.12-4.08 (m, 1H), 3.80-3.75 (m, 3H), 3.62-3.40 (m, 3H), 3.32 (s, 3H), 2.60-2.58 (m, 1H), 2.54 (s, 3H), 2.41-2.35 (m, 1H). [M+H] Calcd.: 469.3; Found, 469.3.

### Example 30: 1-((2R,4S)-4-(4-amino-3-((1-cyclopropyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one

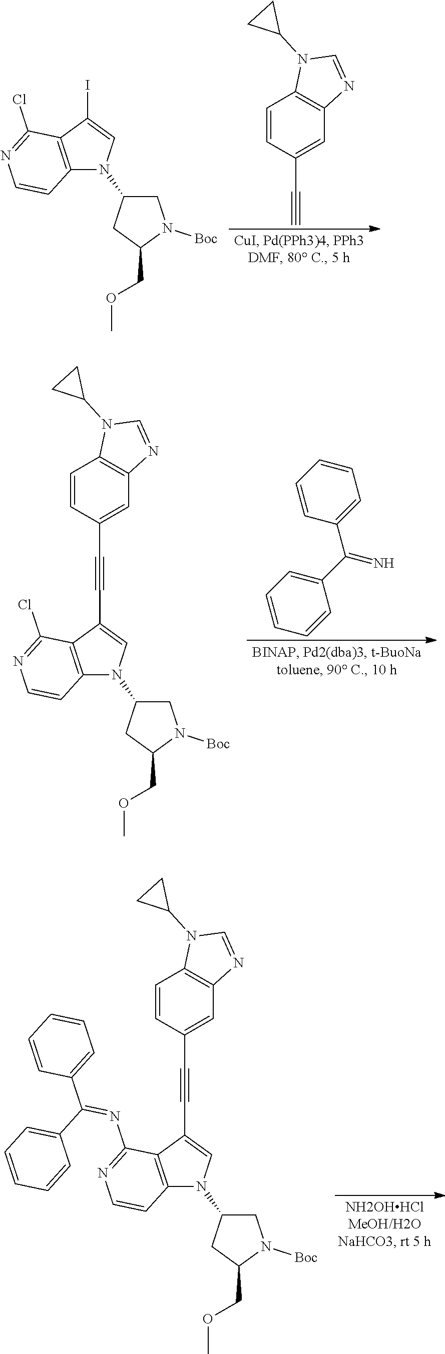 [SMILES: COC[C@H]1C[C@H](n2cc(I)c3c(Cl)nccc32)CN1C(=O)OC(C)(C)C ; C#Cc1ccc2c(c1)ncn2C1CC1 ; COC[C@H]1C[C@H](n2cc(C#Cc3ccc4c(c3)ncn4C3CC3)c3c(Cl)nccc32)CN1C(=O)OC(C)(C)C ; N=C(c1ccccc1)c1ccccc1 ; COC[C@H]1C[C@H](n2cc(C#Cc3ccc4c(c3)ncn4C3CC3)c3c(N=C(c4ccccc4)c4ccccc4)nccc32)CN1C(=O)OC(C)(C)C]

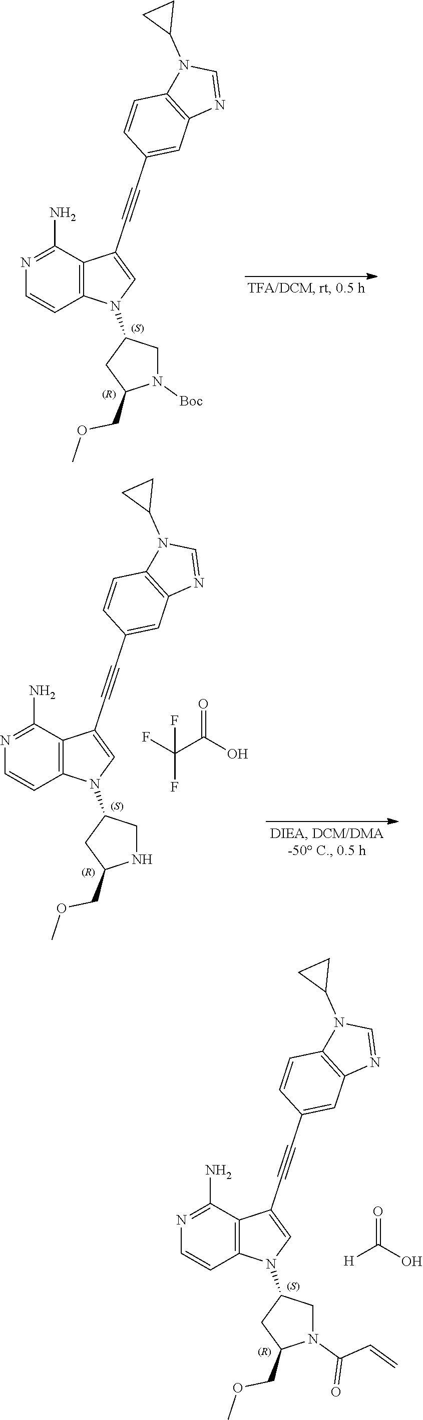

### Step 1: (2R,4S)-tert-butyl 4-(4-chloro-3-((1-cyclopropyl-1H-benzo[d]imidazol-5-yl)ethynyl)-1H-pyrrolo[3,2-c]pyridin-1-yl)-2-(methoxymethyl)pyrrolidine-1-carboxylate

To a mixture of (2R,4S)-tert-butyl 4-(4-chloro-3-iodo-1H-pyrrolo[3,2-c]pyridin-1-yl)-2-(methoxymethyl)pyrrolidine-1-carboxylate (660 mg, 1.34 mmol), 1-cyclopropyl-5-ethynyl-1H-benzo[d]imidazole (290 mg, 1.60 mmol), CuI (76 mg, 0.40 mmol) in DMF (20 mL) was added Pd(PPh3)4 (150 mg, 0.13 mmol), PPh3 (34 mg, 0.13 mmol) and DIEA (516 mg, 4.02 mmol) under nitrogen atmosphere and stirred at 80° C. for 5 h. After cooling down to room temperature, the reaction mixture was diluted with water (60 mL) and extracted with EA (30 mL) for three times, the organic layers were dried over Na2SO4, filtered and concentrated in vacuo. The residue was purified by flash (DCM/MeOH=50/1) to afford (2R,4S)-tert-butyl 4-(4-chloro-3-((1-cyclopropyl-1H-benzo[d]imidazol-5-yl)ethynyl)-1H-pyrrolo[3,2-c]pyridin-1-yl)-2-(methoxymethyl)pyrrolidine-1-carboxylate (680 mg, 81%) as a yellow solid. [M+H] Calcd.: 546.2; Found, 546.2.

### Step 2: (2R,4S)-tert-butyl 4-(3-((1-cyclopropyl-1H-benzo[d]imidazol-5-yl)ethynyl)-4-((diphenylmethylene)amino)-1H-pyrrolo[3,2-c]pyridin-1-yl)-2-(methoxymethyl)pyrrolidine-1-carboxylate

A solution of (2R,4S)-tert-butyl 4-(4-chloro-3-((1-cyclopropyl-1H-benzo[d]imidazol-5-yl)ethynyl)-1H-pyrrolo[3,2-c]pyridin-1-yl)-2-(methoxymethyl)pyrrolidine-1-carboxylate (680 mg, 1.25 mmol), diphenylmethanimine (453 mg, 2.50 mmol), Pd2(dba)3 (230 mg, 0.25 mmol), BINAP (156 mg, 0.25 mmol) and t-BuONa (360 mg, 3.25 mmol) in toluene (30 mL) was stirred at 90° C. for 10 h under nitrogen atmosphere. After cooling down to room temperature, the reaction mixture was diluted with water (20 mL) and extracted with EA (30 mL) for three times, the combined organic layers were dried over Na2SO4, filtered and concentrated in vacuo. The residue was purified by flash (DCM/MeOH=20/1) to afford (2R,4S)-tert-butyl 4-(3-((1-cyclopropyl-1H-benzo[d]imidazol-5-yl)ethynyl)-4-((diphenylmethylene)amino)-1H-pyrrolo[3,2-c]pyridin-1-yl)-2-(methoxymethyl)pyrrolidine-1-carboxylate (570 mg, 66%) as a yellow solid. [M+H] Calcd.: 691.2; Found, 691.2.

### Step 3: (2R,4S)-tert-butyl 4-(4-amino-3-((1-cyclopropyl-1H-benzo[d]imidazol-5-yl)ethynyl)-1H-pyrrolo[3,2-c]pyridin-1-yl)-2-(methoxymethyl)pyrrolidine-1-carboxylate

To a solution of (2R,4S)-tert-butyl 4-(3-((1-cyclopropyl-1H-benzo[d]imidazol-5-yl)ethynyl)-4-((diphenylmethylene)amino)-1H-pyrrolo[3,2-c]pyridin-1-yl)-2-(methoxymethyl)pyrrolidine-1-carboxylate (570 mg, 0.83 mmol) in MeOH (20 mL) and H2O (20 mL) was added hydroxylamine hydrochloride (1.40 g, 20.7 mmol) and NaHCO₃ (1.70 g, 20.7 mmol) and stirred at rt for 5 h. The reaction mixture was diluted with water (20 mL) and extracted with EA (10 mL) for three times, the combined organic layers were dried over Na2SO4, filtered and concentrated in vacuo. The residue was purified by flash (DCM/MeOH=10/1) to afford (2R,4S)-tert-butyl 4-(4-amino-3-((1-cyclopropyl-1H-benzo[d]imidazol-5-yl)ethynyl)-1H-pyrrolo[3,2-c]pyridin-1-yl)-2-(methoxymethyl)pyrrolidine-1-carboxylate (310 mg, 71%) as a yellow solid. [M+H] Calcd.: 527.2 Found, 527.2.

### Step 4: 3-((1-cyclopropyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate

To a solution of (2R,4S)-tert-butyl 4-(4-amino-3-((1-cyclopropyl-1H-benzo[d]imidazol-5-yl)ethynyl)-1H-pyrrolo[3,2-c]pyridin-1-yl)-2-(methoxymethyl)pyrrolidine-1-carboxylate (310 mg, 0.59 mmol) in DCM (2 mL) was added TFA (2 mL) at room temperature. The reaction mixture was stirred at room temperature for 0.5 h. The reaction mixture was concentrated under vacuum to give 3-((1-cyclopropyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate (270 mg, crude) as a yellow solid. [M+H] Calcd.: 427.2; Found, 427.2.

### Step 5: 1-((2R,4S)-4-(4-amino-3-((1-cyclopropyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate

To a solution of 3-((1-cyclopropyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate (270 mg, 0.57 mmol), DIEA (221 mg, 1.71 mmol) in DCM (2 mL) and DMA (2 mL) at −50° C. under nitrogen atmosphere was added acryloyl chloride (52 mg, 0.57 mmol) in DCM (0.5 mL) slowly. The mixture was stirred at −50° C. for 0.5 h. The reaction mixture was quenched with water (5 mL) and extracted with DCM (10 mL) for three times, the combined organic layers was dried over Na2SO4, filtered and concentrated in vacuo which was purified by prep-HPLC to afford 1-((2R,4S)-4-(4-amino-3-((1-cyclopropyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate (79.6 mg, 29%) as a yellow solid. 1H NMR (400 MHz, DMSO-d6): 8.32 (s, 1H), 8.15 (s, 1H), 7.88-7.83 (m, 2H), 7.72-7.65 (m, 2H), 7.47 (dd, J=1.2, 8.4 Hz, 1H), 7.04 (d, J=6.4 Hz, 1H), 6.80-6.53 (m, 3H), 6.21-6.15 (m, 1H), 5.74-5.67 (m, 1H), 5.35-5.27 (m, 1H), 4.64-4.45 (m, 1H), 4.13-4.08 (m, 1H), 3.82-3.75 (m, 2H), 3.62-3.46 (m, 2H), 3.33 (s, 3H), 2.64-2.52 (m, 1H), 243-2.36 (m, 1H), 1.14-1.02 (m, 4H). [M+H] Calcd.: 481.3; Found, 481.3.

### Example 31: 1-((2R,4S)-4-(4-amino-3-((1-ethyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one

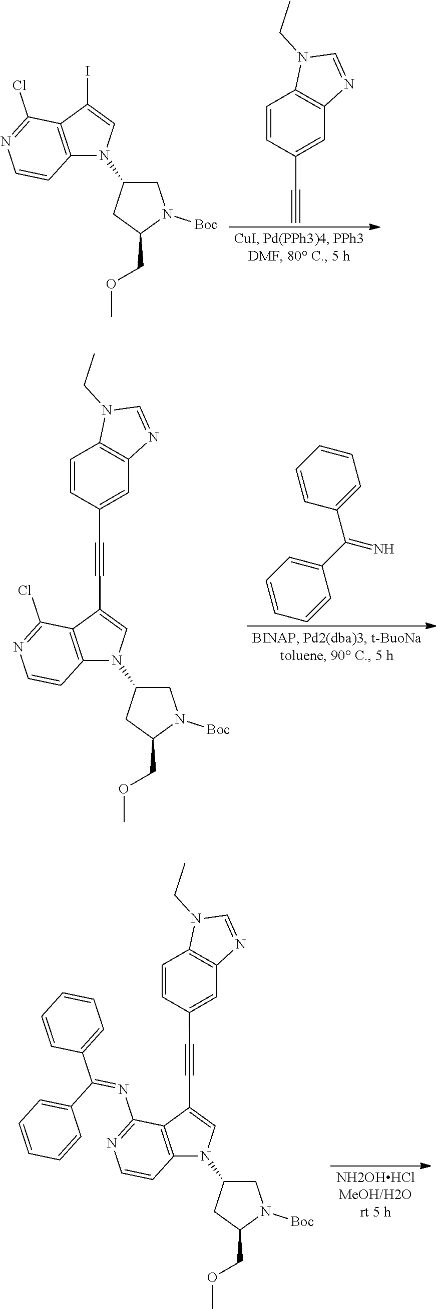

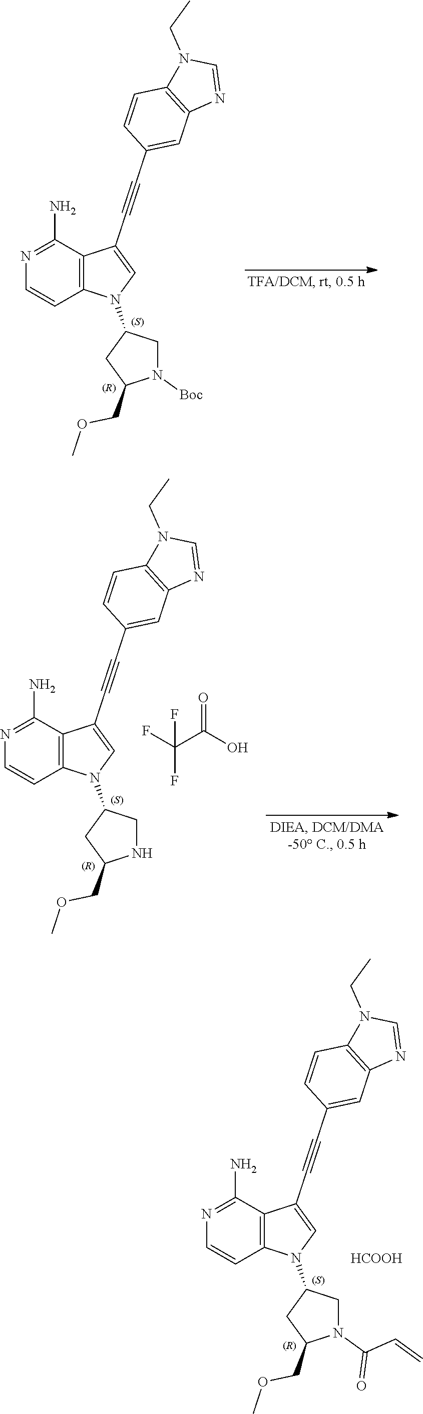

### Step 1: (2R,4S)-tert-butyl 4-(4-chloro-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate

To a mixture of (2R,4S)-tert-butyl 4-(4-chloro-3-iodo-1H-pyrrolo[3,2-c]pyridin-1-yl)-2-(methoxymethyl)pyrrolidine-1-carboxylate (900 mg, 1.8 mmol), 1-ethyl-5-ethynyl-1H-benzo[d]imidazole (374 mg, 2.2 mmol), CuI (103 mg, 0.54 mmol) in DMF (10 mL) was added Pd(PPh3)4 (208 mg, 0.18 mmol), PPh3 (47 mg, 0.18 mmol) and DIEA (700 mg, 5.4 mmol) under nitrogen atmosphere and stirred at 80° C. for 5 h. After cooling down to room temperature, the reaction mixture was diluted with water (60 mL) and extracted with EA (30 mL) for three times, the combined organic layers were dried over Na2SO4, filtered and concentrated in vacuo. The residue was purified by flash (DCM/MeOH=20/1) to afford (2R,4S)-tert-butyl 4-(4-chloro-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate (800 mg, 83%) as an orange solid. [M+H] Calcd.: 534.2; Found, 534.2.

### Step 2: (2R,4S)-tert-butyl 4-(4-((diphenylmethylene)amino)-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate

A solution of (2R,4S)-tert-butyl 4-(4-chloro-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate (850 mg, 1.59 mmol), diphenylmethanimine (575 mg, 3.18 mmol), Pd2(dba)3 (293 mg, 0.32 mmol), BINAP (199 mg, 0.32 mmol) and t-BuONa (458 mg, 4.77 mmol) in toluene (20 mL) was stirred at 90° C. for 5 h under nitrogen atmosphere. After cooling down to room temperature, the reaction mixture was diluted with water (20 mL) and extracted with EA (30 mL) for three times, the combined organic layers were dried over Na2SO4, filtered and concentrated in vacuo. The residue was purified by flash (DCM/MeOH=20/1) to afford (2R,4S)-tert-butyl 4-(4-((diphenylmethylene)amino)-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate (700 mg, 65%) as a yellow solid. [M+H] Calcd.: 679.3; Found, 679.3.

### Step 3: (2R,4S)-tert-butyl 4-(4-amino-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate

To a solution of (2R,4S)-tert-butyl 4-(4-((diphenylmethylene)amino)-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate (800 mg, 1.18 mmol) in MeOH (50 mL) and H2O (30 mL) was added hydroxylamine hydrochloride (2.06 g, 29.4 mmol) and NaHCO₃ (2.50 g, 29.4 mmol) and stirred at rt for 5 h. The reaction mixture was diluted with water (20 mL) and extracted with EA (10 mL) for three times, the combined organic layers were dried over Na2SO4, filtered and concentrated in vacuo. The residue was purified by flash (DCM/MeOH=20/1) to afford (2R,4S)-tert-butyl 4-(4-amino-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate (480 mg, 79%) as a yellow solid. [M+H] Calcd.: 515.2 Found, 515.2.

### Step 4: 3-((1-ethyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate

To a solution of 2R,4S)-tert-butyl 4-(4-amino-3-((1-ethyl-1H-benzo[d]imidazol-5-yl)ethynyl)-1H-pyrrolo[3,2-c]pyridin-1-yl)-2-(methoxymethyl)pyrrolidine-1-carboxylate (260 mg, 0.51 mmol) in DCM (4 mL) was added TFA (2 mL) at room temperature. The reaction mixture was stirred at room temperature for 0.5 h. The reaction mixture was concentrated under vacuum to give 3-((1-ethyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate (230 mg, crude) as a yellow solid. [M+H] Calcd.: 415.2; Found, 415.2.

### Step 5: 1-((2R,4S)-4-(4-amino-3-((1-ethyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate

To a solution of 3-((1-ethyl-1H-benzo[d]imidazol-5-yl)ethynyl)-1-((3S,5R)-5-(methoxymethyl)pyrrolidin-3-yl)-1H-pyrrolo[3,2-c]pyridin-4-amine 2,2,2-trifluoroacetate (210 mg, 0.50 mmol), DIEA (194 mg, 1.50 mmol) in DCM (2 mL) and DMA (2 mL) at −50° C. under nitrogen atmosphere was added acryloyl chloride (45 mg, 0.50 mmol) in DCM (0.5 mL) slowly. The mixture was stirred at −50° C. for 0.5 h. The reaction mixture was quenched with water (5 mL) and extracted with DCM (10 mL) for three times, the combined organic layers were dried over Na2SO4, filtered and concentrated in vacuo which was purified by prep-HPLC to afford 1-((2R,4S)-4-(4-amino-3-((1-ethyl-1H-benzo[d]imidazol-5-yl)ethynyl)-1H-pyrrolo[3,2-c]pyridin-1-yl)-2-(methoxymethyl)pyrrolidin-1-yl)prop-2-en-1-one formate (33.7 mg, 14%) as a yellow solid. 1H NMR (400 MHz, DMSO-d6): 8.35 (s, 1H), 8.14 (s, 1H), 7.89-7.86 (m, 2H), 7.73-7.67 (m, 2H), 7.44 (dd, J=1.6, 8.4 Hz, 1H), 7.09 (d, J=6.4 Hz, 1H), 6.80-6.53 (m, 3H), 6.21-6.15 (m, 1H), 5.74-5.67 (m, 1H), 5.36-5.28 (m, 1H), 4.64-4.45 (m, 1H), 4.30 (q, J=7.2 Hz, 2H), 4.13-3.73 (m, 2H), 3.62-3.44 (m, 2H), 3.33 (s, 3H), 2.67-2.61 (m, 1H), 249-2.39 (m, 1H), 1.42 (t, J=7.2 Hz, 3H). [M+H] Calcd.: 469.2; Found, 469.2.

### Example 32: 1-((2R,4S)-4-(4-amino-3-((1-ethyl-4,6-difluoro-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

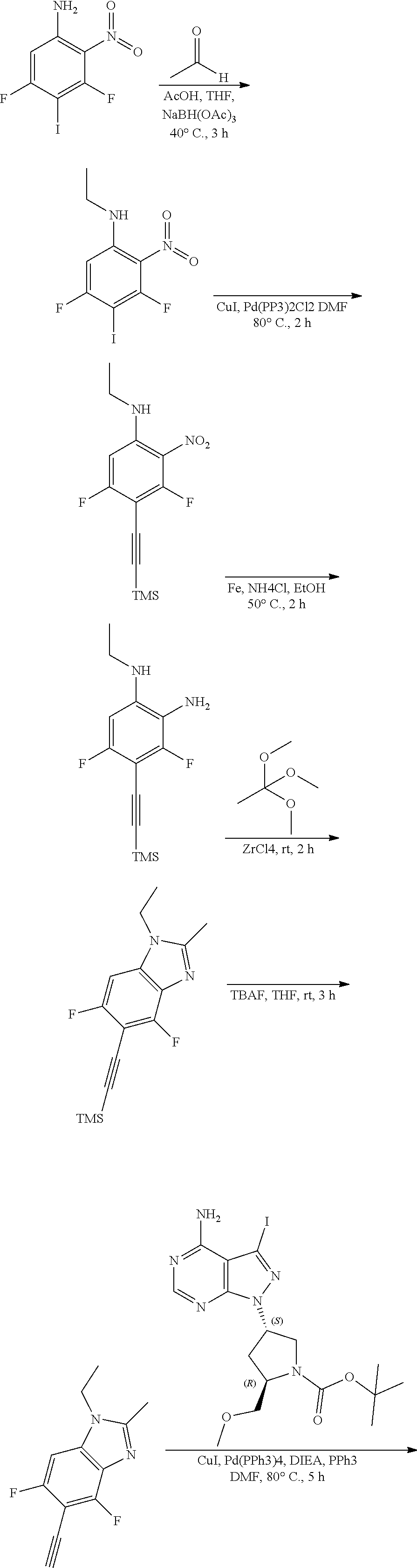

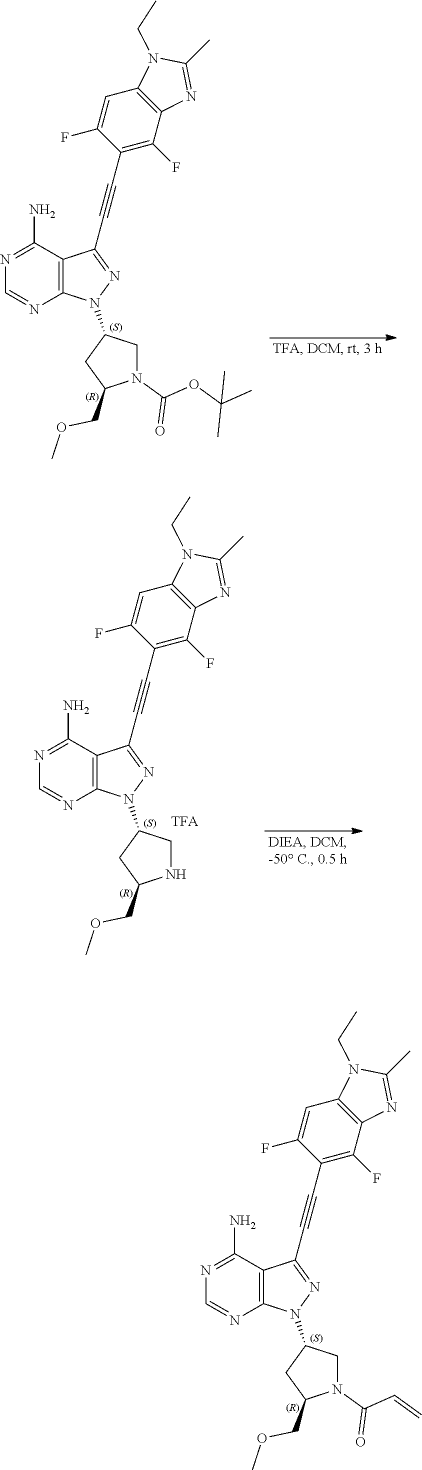

### Step 1: N-ethyl-3,5-difluoro-4-iodo-2-nitroaniline

To a solution of 3,5-difluoro-4-iodo-2-nitroaniline (3.0 g, 10.0 mmol) in AcOH (10 mL) and THF (10 mL) was added acetaldehyde (440 mg, 10.0 mmol) and stirred at 30° C. for 20 min. Then the mixture was added NaBH(OAc)3 (4.20 g, 20.0 mmol) and stirred for 3 h at 40° C. The reaction mixture was quenched with sat. NH4Cl (20 mL) aq and extracted with EA (50 mL) for three times, the combined organic layers were dried over Na2SO4, filtered and concentrated in vacuo to give a residue, which was purified by flash (PE/EA=10/1) to afford N-ethyl-3,5-difluoro-4-iodo-2-nitroaniline (2.3 g, 70%) as a yellow solid. [M+H] Calcd.: 329.0; Found, 329.0.

### Step 2: N-ethyl-3,5-difluoro-2-nitro-4-((trimethylsilyl)ethynyl)aniline

To a mixture of N-ethyl-3,5-difluoro-4-iodo-2-nitroaniline (2.30 g, 7.01 mmol), CuI (133 mg, 0.70 mmol), Pd(PPh3)2Cl2 (246 mg, 0.35 mmol) in DMF (20 mL) was added ethynyltrimethylsilane (1.37 g, 14.0 mmol) and TEA (1.35 g, 10.5 mmol). The reaction mixture was stirred at 80° C. for 2 h under nitrogen atmosphere. After cooling down to room temperature, the solvent was removed to give a residue, which was purified by flash (PE/EA=5/1) to afford N-ethyl-3,5-difluoro-2-nitro-4-((trimethylsilyl)ethynyl)aniline (1.0 g, 50%) as a brown solid. [M+H] Calcd.: 299.1; Found, 299.1.

### Step 3: N1-ethyl-3,5-difluoro-4-((trimethylsilyl)ethynyl)benzene-1,2-diamine

To a solution of N-ethyl-3,5-difluoro-2-nitro-4-((trimethylsilyl)ethynyl)aniline (1.0 g, 3.35 mmol) and NH4Cl (1.8 g, 33.5 mmol) in EtOH (20 mL) and H2O (20 mL) was added iron (1.9 g, 33.5 mmol) at 50° C. After stirred at 50° C. for 2 h, the mixture was filtered and concentrated, the residue was purified by flash (PE/EA=10/1) to give N1-ethyl-3,5-difluoro-4-((trimethylsilyl)ethynyl)benzene-1,2-diamine (700 mg, 77%) as a white solid. [M+H] Calcd.: 269.1; Found, 269.1.

### Step 4: 1-ethyl-4,6-difluoro-2-methyl-5-((trimethylsilyl)ethynyl)-1H-benzo[d]imidazole

To a solution of N1-ethyl-3,5-difluoro-4-((trimethylsilyl)ethynyl)benzene-1,2-diamine (700 mg, 2.61 mmol) in MeOH (20 mL) was added 1,1,1-trimethoxyethane (508 mg, 3.15 mmol) and ZrCl4 (61 mg, 0.26 mmol) at 0° C. After stirred at room temperature for 2 h, the mixture was filtered and concentrated, the residue was purified by flash (PE/EA=1/3) to give 1-ethyl-4,6-difluoro-2-methyl-5-((trimethylsilyl)ethynyl)-1H-benzo[d]imidazole (670 mg, 87%) as off-white solid. [M+H] Calcd.: 293.1; Found, 293.1.

### Step 5: 1-ethyl-5-ethynyl-4,6-difluoro-2-methyl-1H-benzo[d]imidazole

To a solution of 1-ethyl-4,6-difluoro-2-methyl-5-((trimethylsilyl)ethynyl)-1H-benzo[d]imidazole (670 mg, 2.29 mmol) in THF (20 mL) was added 1 M TBAF in THF (2.75 mL, 2.75 mmol). The reaction mixture was stirred at room temperature for 3 h. The solvent was removed to give a residue, which was purified by flash (PE/EA=1/1) to afford 1-ethyl-5-ethynyl-4,6-difluoro-2-methyl-1H-benzo[d]imidazole (430 mg, 85%) as a yellow solid. [M+H] Calcd.: 221.1; Found, 221.1.

### Step 6: (2R,4S)-tert-butyl 4-(4-amino-3-((1-ethyl-4,6-difluoro-2-methyl-1H-benzo[d]imidazol-5-yl)ethynyl)-1H-pyrazolo[3,4-d]pyrimidin-1-yl)-2-(methoxymethyl)pyrrolidine-1-carboxylate

To a mixture of (2R,4S)-tert-butyl 4-(4-amino-3-iodo-1H-pyrazolo[3,4-d]pyrimidin-1-yl)-2-(methoxymethyl)pyrrolidine-1-carboxylate (200 mg, 0.42 mmol), 1-ethyl-5-ethynyl-4,6-difluoro-2-methyl-1H-benzo[d]imidazole (112 mg, 0.50 mmol), CuI (12 mg, 0.06 mmol) in DMF (10 mL) was added Pd(PPh3)4 (243 mg, 0.21 mmol), PPh3 (6 mg, 0.02 mmol) and DIEA (163 mg, 1.26 mmol) under nitrogen atmosphere and stirred at 80° C. for 5 h. After cooling down to room temperature, the reaction mixture was diluted with water (20 mL) and extracted with DCM (20 mL) for three times, the organic layers were dried over Na2SO4, filtered and concentrated in vacuo which was purified by flash (DCM/MeOH=10/1) to afford (2R,4S)-tert-butyl 4-(4-amino-3-((1-ethyl-4,6-difluoro-2-methyl-1H-benzo[d]imidazol-5-yl)ethynyl)-1H-pyrazolo[3,4-d]pyrimidin-1-yl)-2-(methoxymethyl)pyrrolidine-1-carboxylate (130 mg, 54%) as a brown solid. [M+H] Calcd.: 567.3; Found, 567.3.

### Step 7: 3-((1-ethyl-4,6-difluoro-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine 2,2,2-trifluoroacetate

To a solution of (2R,4S)-tert-butyl 4-(4-amino-3-((1-ethyl-4,6-difluoro-2-methyl-1H-benzo[d]imidazol-5-yl)ethynyl)-1H-pyrazolo[3,4-d]pyrimidin-1-yl)-2-(methoxymethyl)

pyrrolidine-1-carboxylate (130 mg, 0.23 mmol) in DCM (10 mL) was added TFA (0.5 mL), the mixture was stirred at room temperature for 3 h. The reaction mixture was concentrated in vacuo to afford 3-((1-ethyl-4,6-difluoro-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine 2,2,2-trifluoroacetate (107 mg, crude) as yellow oil. [M+H] Calcd.: 467.2; Found, 467.2.

### Step 8: 1-((2R,4S)-4-(4-amino-3-((1-ethyl-4,6-difluoro-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a solution of 3-((1-ethyl-4,6-difluoro-2-methyl-1H-benzo[d]imidazol-5-yl)ethynyl)-1-((3S,5R)-5-(methoxymethyl)pyrrolidin-3-yl)-1H-pyrazolo[3,4-d]pyrimidin-4-amine 2,2,2-trifluoroacetate (105 mg, 0.22 mmol), DIEA (142 mg, 1.11 mmol) in DCM (15 mL) at −50° C. under nitrogen atmosphere was added acryloyl chloride (18 mg, 0.20 mmol) in DCM (0.5 mL) slowly. The mixture was stirred at −50° C. for 0.5 h. The reaction mixture was quenched with water (15 mL) and extracted with DCM (10 mL) twice. The combined organic layers were dried over Na2SO4, filtered and concentrated in vacuo to give a residue, which was purified by prep-HPLC to afford 1-((2R,4S)-4-(4-amino-3-((1-ethyl-4,6-difluoro-2-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (19.2 mg, 42%) as a white solid. 1H NMR (400 MHz, CDCl3): 8.42-8.36 (m, 1H), 6.93 (d, J=8.0 Hz, 1H), 6.65-6.40 (m, 2H), 6.04 (br s, 2H), 5.80-5.65 (m, 2H), 4.71-4.48 (m, 1H), 4.19-3.96 (m, 4H), 3.86-3.78 (m, 1H), 3.57-3.48 (m, 1H), 3.40 (s, 3H), 3.03-2.78 (m, 1H), 2.63 (s, 3H), 2.52-2.42 (m, 1H), 1.43 (t, J=7.2 Hz, 3H). [M+H] Calcd.: 521.2; Found, 521.2.

### Example 33: 1-((2R,4S)-4-(4-amino-3-((1-ethyl-4,6-difluoro-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

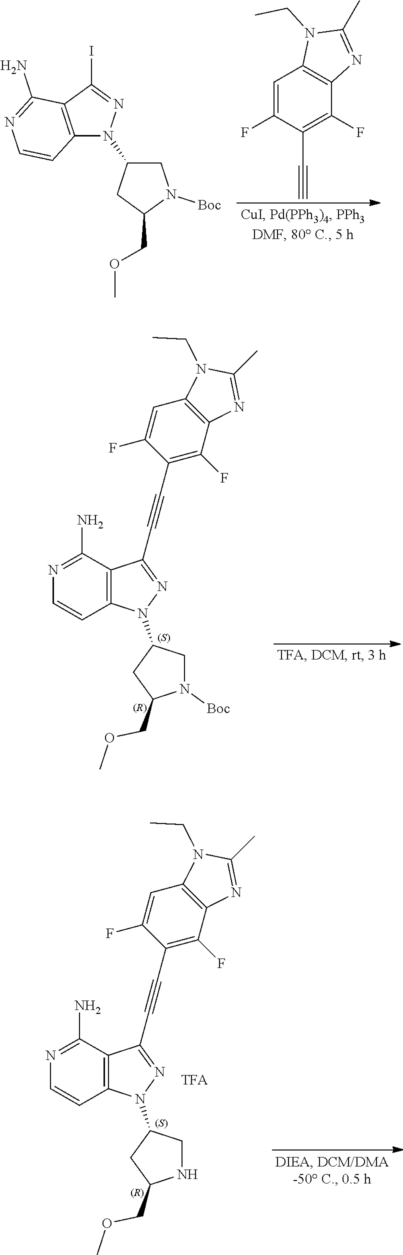

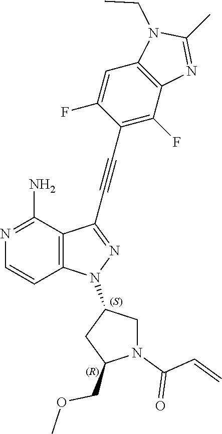

### Step 1: (2R,4S)-tert-butyl 4-(4-amino-3-((1-ethyl-4,6-difluoro-2-methyl-1H-benzo[d]imidazol-5-yl)ethynyl)-1H-pyrazolo[4,3-c]pyridin-1-yl)-2-(methoxymethyl)pyrrolidine-1-carboxylate

To a mixture of (2R,4S)-tert-butyl 4-(4-amino-3-iodo-1H-pyrazolo[4,3-c]pyridin-1-yl)-2-(methoxymethyl)pyrrolidine-1-carboxylate (200 mg, 0.42 mmol), 1-ethyl-5-ethynyl-4,6-difluoro-2-methyl-1H-benzo[d]imidazole (112 mg, 0.50 mmol), CuI (76 mg, 0.40 mmol) in DMF (10 mL) was added Pd(PPh3)4 (242 mg, 0.21 mmol), PPh3 (6 mg, 0.021 mmol) and DIEA (163 mg, 1.26 mmol) under nitrogen atmosphere and stirred at 80° C. for 5 h. After cooling down to room temperature, the reaction mixture was diluted with water (60 mL) and extracted with EA (30 mL) for three times, the organic layers were dried over Na2SO4, filtered and concentrated in vacuo. The residue was purified by flash (100% EA) to afford (2R,4S)-tert-butyl 4-(4-amino-3-((1-ethyl-4,6-difluoro-2-methyl-1H-benzo[d]imidazol-5-yl)ethynyl)-1H-pyrazolo[4,3-c]pyridin-1-yl)-2-(methoxymethyl)pyrrolidine-1-carboxylate (120 mg, 50%) as a yellow solid. [M+H] Calcd.: 566.3; Found, 566.3.

### Step 2: 3-((1-ethyl-4,6-difluoro-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate

To a solution of (2R,4S)-tert-butyl 4-(4-amino-3-((1-ethyl-4,6-difluoro-2-methyl-1H-benzo[d]imidazol-5-yl)ethynyl)-1H-pyrazolo[4,3-c]pyridin-1-yl)-2-(methoxymethyl)pyrrolidine-1-carboxylate (120 mg, 0.21 mmol) in DCM (10 mL) was added TFA (1 mL) at room temperature. The reaction mixture was stirred at room temperature for 3 h. The reaction mixture was concentrated under vacuum to afford 3-((1-ethyl-4,6-difluoro-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (98 mg, crude) as a yellow solid. [M+H] Calcd.: 466.3; Found, 466.3.

### Step 3: 1-((2R,4S)-4-(4-amino-3-((1-ethyl-4,6-difluoro-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

To a solution of 3-((1-ethyl-4,6-difluoro-2-methyl-1H-benzo[d]imidazol-5-yl)ethynyl)-1-((3S,5R)-5-(methoxymethyl)pyrrolidin-3-yl)-1H-pyrazolo[4,3-c]pyridin-4-amine 2,2,2-trifluoroacetate (95 mg, 0.20 mmol), DIEA (129 mg, 1.00 mmol) in DCM (15 mL) and DMA (5 mL) at −50° C. under nitrogen atmosphere was added acryloyl chloride (17 mg, 0.18 mmol) in DCM (0.5 mL) slowly. The mixture was stirred at −50° C. for 0.5 h. The reaction mixture was quenched with water (5 mL) and extracted with DCM (10 mL) twice. The combined organic layers were dried over Na2SO4, filtered and concentrated in vacuo to give a residue, which was purified by prep-HPLC to afford 1-((2R,4S)-4-(4-amino-3-((1-ethyl-4,6-difluoro-2-methyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one (45.4 mg, 42%) as a white solid. 1H NMR (400 MHz, DMSO-d6): 7.82 (d, J=6.0 Hz, 1H), 7.84 (d, J=8.4 Hz, 1H), 6.97 (d, J=6.4 Hz, 1H), 6.79-6.50 (m, 3H), 6.20-6.14 (m, 1H), 5.72-5.65 (m, 1H), 5.52-5.47 (m, 1H), 4.66-4.52 (m, 1H), 4.25 (q, J=7.2 Hz, 2H), 4.12-3.80 (m, 2H), 3.63-3.48 (m, 2H), 3.33 (s, 3H), 2.72-2.52 (m, 1H), 2.61 (s, 3H), 2.43-2.38 (m, 1H), 1.30 (t, J=7.2 Hz, 3H). [M+H] Calcd.: 520.2; Found, 520.2.

### Example 34: 1-((2R,4S)-4-(4-amino-3-((1-ethyl-1H-indazol-5-yl)ethynyl)-1H-pyrazolo[3,4-d]pyrimidin-1-yl)-2-(methoxymethyl)pyrrolidin-1-yl)prop-2-en-1-one

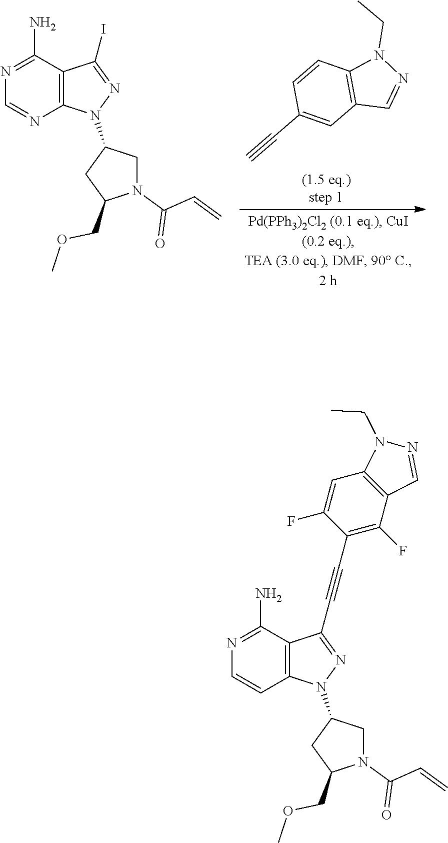 [SMILES: C=CC(=O)N1C[C@@H](n2nc(I)c3c(N)ncnc32)C[C@@H]1COC ; C#Cc1ccc2c(cnn2CC)c1 ; C=CC(=O)N1C[C@@H](n2nc(C#Cc3c(F)cc4c(cnn4CC)c3F)c3c(N)nccc32)C[C@@H]1COC]

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.10 g, 0.23 mmol) and 1-ethyl-5-ethynylindazole (59.62 mg, 0.35 mmol) in DMF (3.00 mL) were added Pd(PPh3)4 (26.9 mg, 0.02 mmol), CuI (8.89 mg, 0.05 mmol) and TEA (70.89 mg, 0.70 mmol) at room temperature. The reaction mixture was degassed with argon for three times and stirred for 2 h at 90° C. The resulting mixture was concentrated under reduced pressure. The residue was purified by Prep-HPLC with the following conditions: Column: Atlantis T3 OBD Prep Column, 10 μm, 19 mm×250 mm; Mobile Phase A: Water (0.1% FA), Mobile Phase B: ACN; Flow rate: 20 mL/min; Gradient: 25% B to 55% B in 6 min; Detector: UV 254 & 210 nm; RT: 5.58 min. The fractions that contained desired product were combined and concentrated to afford 1-[(2R,4S)-4-[4-amino-3-[2-(1-ethylindazol-5-yl)ethynyl]pyrazolo[3,4-d]pyrimidin-1-yl]-2-(methoxymethyl)pyrrolidin-1-yl]prop-2-en-1-one (39.5 mg, 34%) as a light yellow solid. MS ESI calculated for C25H26N8O₂ [M+H]+, 471.22 found 471.10. 1H-NMR (300 MHz, d6-DMSO) δ 8.49-8.08 (m, 3H), 7.81-7.67 (m, 2H), 6.83-6.55 (m, 1H), 6.19-6.13 (m, 1H), 5.82-5.57 (m, 2H), 4.48 (q, J=7.2 Hz, 3H), 4.11-3.92 (m, 1H), 3.66-3.43 (m, 6H), 2.71-2.55 (m, 1H), 2.43-2.30 (m, 1H), 1.41 (t, J=7.2 Hz, 3H).

### Example 35: 1-[(2R,4S)-4-[4-amino-3-[2-(1-methylindazol-5-yl)ethynyl]pyrazolo[4,3-c]pyridin-1-yl]-2-(methoxymethyl)pyrrolidin-1-yl]prop-2-en-1-one

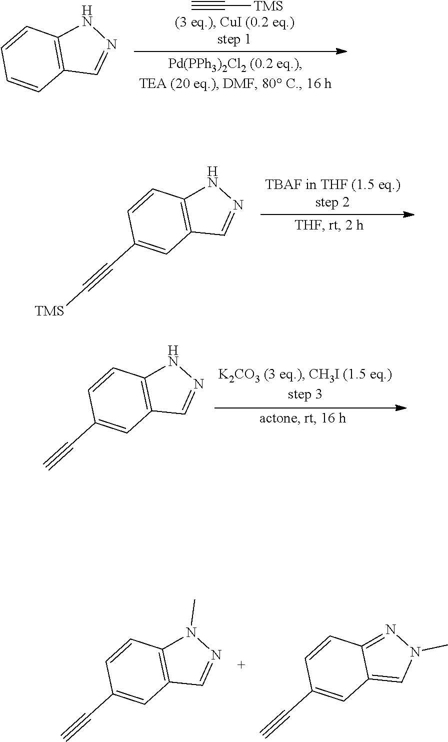

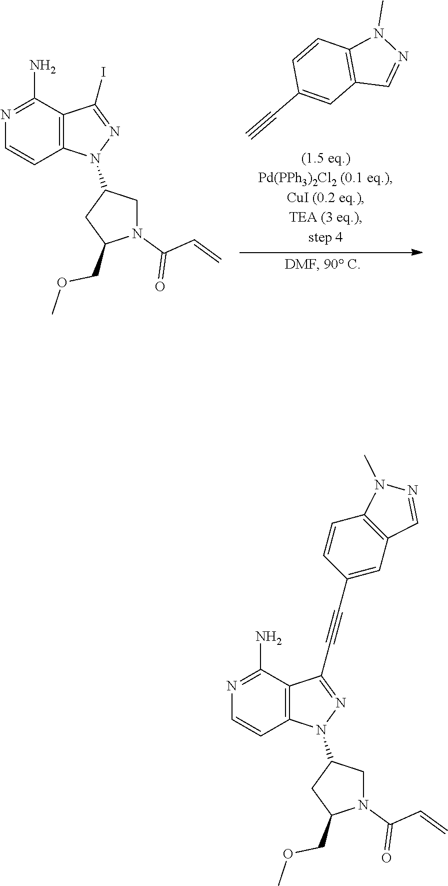

### Step 1: 5-[2-(trimethylsilyl)ethynyl]-1H-indazole

To a stirred mixture of 5-bromo-1H-indazole (5.00 g, 25.38 mmol), trimethylsilylacetylene (10.76 mL, 109.54 mmol), CuI (0.97 g, 5.08 mmol) and Pd(PPh3)2Cl2 (3.56 g, 5.08 mmol) in DMF (100.00 mL) was added TEA (70.54 mL, 697.15 mmol) dropwise at room temperature. The reaction mixture was degassed with argon for three times and stirred for 16 h at 80° C. The resulting mixture was diluted with water (300 mL), extracted with EtOAc (3×200 mL). The combined organic layers were washed with brine (2×300 mL), dried over anhydrous Na2SO4 and filtered. The filtrate was concentrated under reduced pressure. The residue was purified by silica gel column chromatography, eluted with PE/EtOAc (4:1). The fractions that contained desired product were combined and concentrated to afford 5-[2-(trimethylsilyl)ethynyl]-1H-indazole (4.68 g, 68%) as a brown solid. MS ESI calculated for C12H14N2Si [M+H]+, 215.09, found 215.20.

### Step 2: 5-ethynyl-1H-indazole

To a stirred solution of 5-[2-(trimethylsilyl)ethynyl]-1H-indazole (4.68 g, 21.83 mmol) in THF (45.00 mL) was added TBAF (32.19 mL, 32.19 mmol) dropwise at 0° C. under nitrogen atmosphere. The reaction mixture was stirred for 2 h at room temperature. The resulting mixture was diluted with water (100 mL), extracted with EtOAc (3×150 mL). The combined organic layers were washed with brine (200 mL), dried over anhydrous Na2SO4 and filtered. The filtrate was concentrated under reduced pressure. The residue was purified by silica gel column chromatography, eluted with PE/EtOAc (3:1). The fractions that contained desired product were combined and concentrated to afford 5-ethynyl-1H-indazole (2.3 g, 66%) as an off-white solid. MS ESI calculated for C9H6N2 [M+H]+, 143.05, found 143.20.

### Step 3: 5-ethynyl-1-methylindazole and 5-ethynyl-2-methylindazole

To a stirred solution of 5-ethynyl-1H-indazole (0.80 g, 5.62 mmol) and K2CO3 (2.33 g, 16.88 mmol) in acetone (16.00 mL) was added CH3I (0.53 mL, 3.70 mmol) dropwise at 0° C. under nitrogen atmosphere. The reaction mixture was stirred for 16 h at room temperature. The resulting mixture was filtered, the filter cake was washed with ethyl acetate (3×40 mL). The filtrate was concentrated under reduced pressure. The residue was purified by silica gel column chromatography, eluted with 23% EA in PE. The fractions that contained desired product were combined and concentrated to afford 5-ethynyl-1-methylindazole (0.50 g, 54%) as an off-white solid. MS ESI calculated for C10H8N2 [M+H]+, 157.07, found 157.15. Also eluted with 40% EA in PE. The fractions that contained desired product were combined and concentrated to afford 5-ethynyl-2-methylindazole (0.30 g, 30%) as an off-white solid. MS ESI calculated for C10H8N2 [M+H]+, 157.07, found 157.10.

### Step 4: 1-[(2R,4S)-4-[4-amino-3-[2-(1-methylindazol-5-yl)ethynyl]pyrazolo[4,3-c]pyridin-1-yl]-2-(methoxymethyl)pyrrolidin-1-yl]prop-2-en-1-one

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (0.1 g, 0.23 mmol), 5-ethynyl-1-methylindazole (54.84 mg, 0.35 mmol), CuI (8.92 mg, 0.05 mmol) and Pd(PPh3)2Cl2 (16.43 mg, 0.02 mmol) in DMF (1.50 mL) was added TEA (0.10 mL, 0.96 mmol) dropwise at room temperature. The reaction mixture was degassed with argon for three times and stirred for 2 h at 90° C. The resulting mixture was diluted with water (30 mL), extracted with EtOAc (3×30 mL). The combined organic layers were washed with brine (2×50 mL), dried over anhydrous Na2SO4 and filtered. The filtrate was concentrated under reduced pressure. The residue was purified by Prep-HPLC with the following conditions Column: Atlantis Prep T3 OBD Column, 19*250 mm 10 um; Mobile Phase A: Water (0.05% FA), Mobile Phase B: ACN; Flow rate: 20 mL/min; Gradient: 20 B to 50 B in 6 min; 210/254 nm; RT1: 5.65. The fractions that contained desired product were combined and concentrated to afford 1-[(2R,4S)-4-[4-amino-3-[2-(1-methylindazol-5-yl)ethynyl]pyrazolo[4,3-c]pyridin-1-yl]-2-(methoxymethyl)pyrrolidin-1-yl]prop-2-en-1-one (55.4 mg, 51%) as a light yellow solid. MS ESI calculated for C25H25N7O2 [M+H]+, 456.21, found 456.15. H-NMR (400 MHz, DMSO-d6): δ 8.22 (s, 1H), 8.16-8.13 (m, 1H), 7.84-7.55 (m, 5H), 7.29-7.24 (m, 1H), 6.78-6.51 (m, 1H), 6.19-6.13 (m, 1H), 5.74-5.57 (m, 2H), 4.61-4.47 (m, 1H), 4.12-4.09 (m, 3H), 3.89-3.63 (m, 2H), 3.58-3.42 (m, 2H), 3.32 (s, 3H), 2.69-2.61 (m, 1H), 2.39-2.32 (m, 1H).

### Example 36: 1-[(2R,4S)-4-[4-amino-3-[2-(2-methylindazol-5-yl)ethynyl]pyrazolo[4,3-c]pyridin-1-yl]-2-(methoxymethyl)pyrrolidin-1-yl]prop-2-en-1-one

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (70.00 mg, 0.16 mmol) in DMF (2.00 mL) were added 5-ethynyl-2-methylindazole (38.38 mg, 0.25 mmol), Pd(PPh3)2Cl2 (11.50 mg, 0.07 mmol), CuI (6.24 mg, 0.03 mmol) and TEA (49.74 mg, 0.492 mmol) at room temperature. The reaction mixture was degassed with argon for three times and stirred for 2 h at 90° C. The resulting mixture was filtered and the filtrate was purified by Prep-HPLC with the following conditions Column: Atlantis Prep T3 OBD Column, 19*250 mm 10 u; Mobile Phase A: Water (0.1% FA), Mobile Phase B: ACN; Flow rate: 20 mL/min; Gradient: 30 B to 55 B in 5.8 min; 210/254 nm; RT1:5.58. The fractions that contained desired product were combined and concentrated to afford 1-[(2R,4S)-4-[4-amino-3-[2-(2-methylindazol-5-yl)ethynyl]pyrazolo[4,3-c]pyridin-1-yl]-2-(methoxymethyl)pyrrolidin-1-yl]prop-2-en-1-one (40.90 mg, 54%) as an off-white solid. MS ESI calculated for C25H25N7O2 [M+H]+ 456.21, found 456.10. H-NMR (300 MHz, d6-DMSO) δ 8.27-8.00 (m, 3H), 7.76 (d, J=8.8 Hz, 2H), 7.62 (dd, J=8.8, 1.5 Hz, 1H), 7.09 (s, 1H), 6.66-6.62 (m, 1H), 6.22-6.10 (m, 1H), 5.76-5.62 (m, 1H), 5.52-5.48 (m, 1H), 4.49-4.43 (m, 3H), 4.10-4.05 (m, 1H), 3.96-3.59 (m, 2H), 3.31 (d, J=3.4 Hz, 3H), 2.45-2.33 (m, 1H), 1.52-1.08 (m, 4H).

### Example 37: 1-((2R,4S)-4-(4-amino-3-((1-ethyl-1H-indazol-5-yl)ethynyl)-1H-pyrazolo[4,3-c]pyridin-1-yl)-2-(methoxymethyl)pyrrolidin-1-yl)prop-2-en-1-one

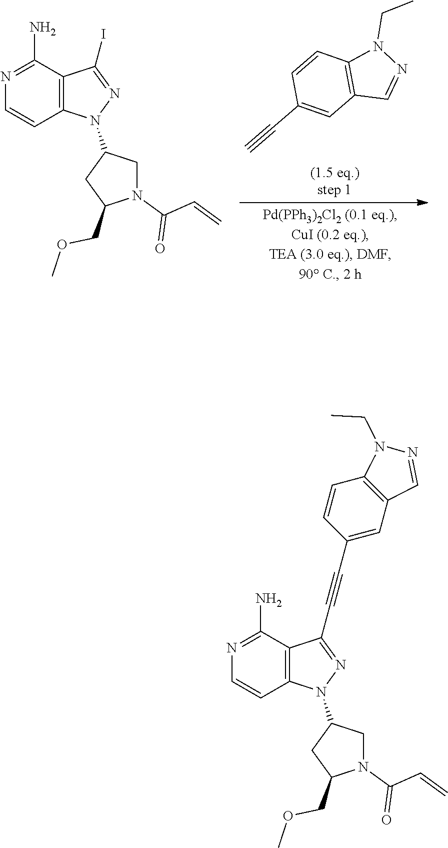

To a solution of 1-[(2R,4S)-4-[4-amino-3-iodopyrazolo[4,3-c]pyridin-1-yl]-2-(methoxymethyl)pyrrolidin-1-yl]prop-2-en-1-one (0.10 g, 0.23 mmol) and 1-ethyl-5-ethynylindazole (59.76 mg, 0.35 mmol) in DMF (3.00 mL) were added Pd(PPh3)2Cl2 (16.43 mg, 0.02 mmol), CuI (8.9 mg, 0.05 mmol) and TEA (71.05 mg, 0.70 mmol) dropwise at room temperature. The reaction mixture was degassed with argon for three times and stirred for 2 h at 90° C. The resulting mixture was concentrated under reduced pressure. The residue was purified by Prep-HPLC with the following conditions: Column: Atlantis T3 OBD Prep Column, 10 μm, 19 mm×250 mm; Mobile Phase A: Water (0.1% FA), Mobile Phase B: ACN; Flow rate: 20 mL/min; Gradient: 30% B to 55% B in 6 min; Detector: UV 254 & 210 nm; RT: 5.58 min. The fractions that contained desired product were combined and concentrated to afford 1-[(2R,4S)-4-[4-amino-3-[2-(1-ethylindazol-5-yl)ethynyl]pyrazolo[4,3-c]pyridin-1-yl]-2-(methoxymethyl)pyrrolidin-1-yl]prop-2-en-1-one (33 mg, 29%) as a light yellow solid. MS ESI calculated for C26H27N7O2 [M+H]+, 470.22 found 470.10. 1H-NMR (400 MHz, DMSO-d6) δ 8.21-8.13 (m, 2H), 7.85 (s, 1H), 7.80 (d, J=8.8 Hz, 1H), 7.65 (dd, J=8.7, 1.5 Hz, 1H), 7.13-7.08 (m, 1H), 6.95-6.90 (m, 1H), 6.59-6.52 (m, 1H), 6.19-6.13 (m, 1H), 5.71-5.66 (m, 1H), 5.55-5.51 (m, 1H), 4.48 (q, J=7.2 Hz, 3H), 4.11-4.07 (m, 1H), 3.94-3.84 (m, 1H), 3.64-3.53 (m, 1H), 3.53-3.47 (m, 1H), 3.45-3.41 (m, 1H), 3.33 (d, J=5.2 Hz, 3H), 2.72-2.62 (m, 1H), 2.43-4.39 (m, 1H), 1.41 (t, J=7.2 Hz, 3H).

### Example 38: 1-[(2R,4S)-4-[4-amino-3-[2-(2-ethylindazol-5-yl)ethynyl]pyrazolo[4,3-c]pyridin-1-yl]-2-(methoxymethyl)pyrrolidin-1-yl]prop-2-en-1-one

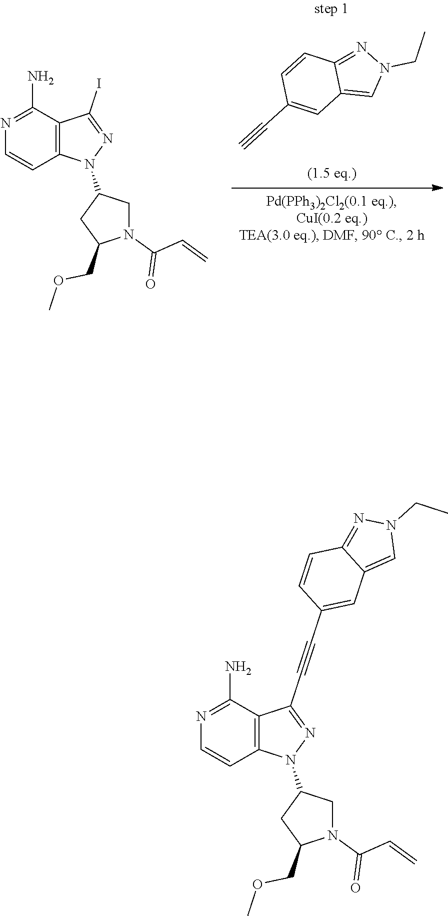

To a stirred solution of 1-[(2R,4S)-4-[4-amino-3-iodopyrazolo[4,3-c]pyridin-1-yl]-2-(methoxymethyl)pyrrolidin-1-yl]prop-2-en-1-one (70.00 mg, 0.16 mmol) in DMF (2.00 mL) were added 2-ethyl-5-ethynylindazole (41.83 mg, 0.25 mmol), Pd(PPh3)2Cl2 (11.50 mg, 0.02 mmol), CuI (6.24 mg, 0.03 mmol) and TEA (49.74 mg, 0.492 mmol). The reaction mixture was degassed with argon for three times and stirred for 2 h at 90° C. The resulting mixture was filtered and the filtrate was purified by Prep-HPLC with the following conditions Column: Atlantis Prep T3 OBD Column, 19*250 mm 10 u; Mobile Phase A: Water (0.1% FA), Mobile Phase B: ACN; Flow rate: 20 mL/min; Gradient: 30 B to 50 B in 6 min; 210/254 nm; RT1:5.58. The fractions that contained desired product were combined and concentrated to afford 1-[(2R,4S)-4-[4-amino-3-[2-(2-ethylindazol-5-yl)ethynyl]pyrazolo[4,3-c]pyridin-1-yl]-2-(methoxymethyl)pyrrolidin-1-yl]prop-2-en-1-one (41.1 mg, 53%) as an off-white solid. MS ESI calculated for C26H27N7O2 [M+H]+ 470.23, found 470.05. H-NMR (300 MHz, d6-DMSO) δ 8.54 (s, 1H), 8.21 (s, 1H), 8.09-7.78 (m, 3H), 7.69 (d, J=8.9 Hz, 1H), 7.56-7.32 (m, 2H), 6.83-6.46 (m, 1H), 6.24-6.10 (m, 1H), 5.79-5.54 (m, 2H), 4.69-4.41 (m, 3H), 4.14-3.79 (m, 2H), 3.74-3.41 (m, 4H), 3.17-3.12 (m, 1H), 2.77-2.62 (m, 1H), 2.43-2.37 (m, 1H), 1.52 (t, J=7.4 Hz, 3H)

### Example 39: 2-((2R,4S)-1-acryloyl-4-(4-amino-3-((4,6-difluoro-1,2-dimethyl-1H-benzo[d]imidazol-5-yl)ethynyl)-1H-pyrazolo[4,3-c]pyridin-1-yl)pyrrolidin-2-yl)acetonitrile

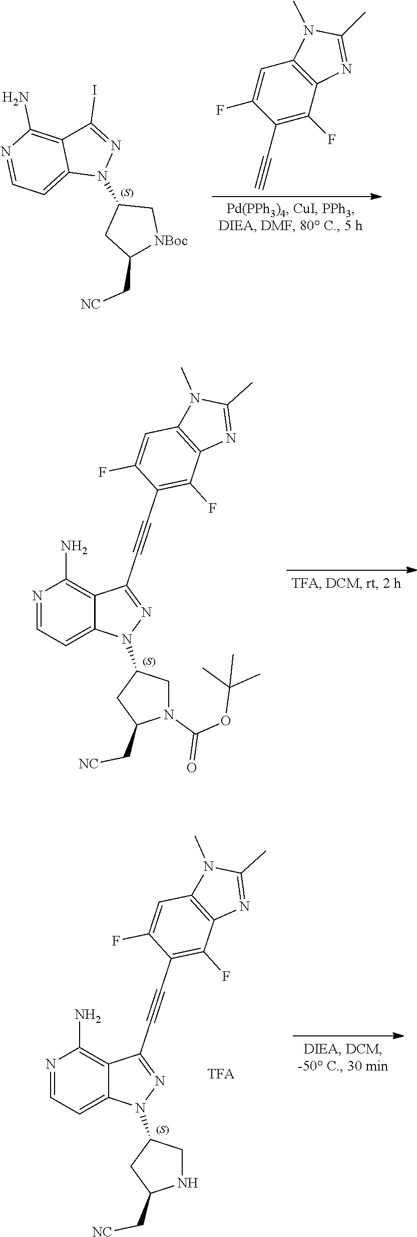

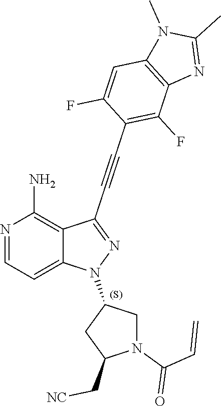

### Step 1: (2R,4S)-tert-butyl 4-(4-amino-3-((4,6-difluoro-1,2-dimethyl-1H-benzo[d]imidazol-5-yl)ethynyl)-1H-pyrazolo[4,3-c]pyridin-1-yl)-2-(cyanomethyl)pyrrolidine-1-carboxylate

To a mixture of (2R,4S)-tert-butyl 4-(4-amino-3-iodo-1H-pyrazolo[4,3-c]pyridin-1-yl)-2-(cyanomethyl)pyrrolidine-1-carboxylate (200 mg, 0.42 mmol), 5-ethynyl-4,6-difluoro-1,2-dimethyl-1H-benzo[d]imidazole (105 mg, 0.51 mmol) and CuI (11 mg, 0.06 mmol) in DMF (10 mL) was added Pd(PPh3)4 (242 mg, 0.21 mmol), PPh3 (6 mg, 0.02 mmol) and DIEA (163 mg, 1.26 mmol) under nitrogen atmosphere and stirred at 80° C. for 5 h. After cooling down to room temperature, the reaction mixture was diluted with water (20 mL) and extracted with DCM (20 mL) for three times, the combined organic layers were dried over anhydrous sodium sulfate, filtered and concentrated in vacuo to give a residue, which was purified by flash (DCM/MeOH=20/1) to afford (2R,4S)-tert-butyl 4-(4-amino-3-((4,6-difluoro-1,2-dimethyl-1H-benzo[d]imidazol-5-yl)ethynyl)-1H-pyrazolo[4,3-c]pyridin-1-yl)-2-(cyanomethyl)pyrrolidine-1-carboxylate (70 mg, 30%) as a yellow solid. [M+H] Calcd.: 547.2; Found, 547.2.

### Step 2: 2-((2R,4S)-4-(4-amino-3-((4,6-difluoro-1,2-dimethyl-1H-benzo[d]imidazol-5-yl)ethynyl)-1H-pyrazolo[4,3-c]pyridin-1-yl)pyrrolidin-2-yl)acetonitrile 2,2,2-trifluoroacetate

To a solution of (2R,4S)-tert-butyl 4-(4-amino-3-((4,6-difluoro-1,2-dimethyl-1H-benzo[d]imidazol-5-yl)ethynyl)-1H-pyrazolo[4,3-c]pyridin-1-yl)-2-(cyanomethyl)pyrrolidine-1-carboxylate (70 mg, 0.12 mmol) in DCM (5 mL) was added TFA (0.5 mL). The reaction mixture was stirred at rt for 2 h. The reaction mixture was concentrated in vacuum to give 2-((2R,4S)-4-(4-amino-3-((4,6-difluoro-1,2-dimethyl-1H-benzo[d]imidazol-5-yl)ethynyl)-1H-pyrazolo[4,3-c]pyridin-1-yl)pyrrolidin-2-yl)acetonitrile 2,2,2-trifluoroacetate (57 mg, crude) as yellow oil. [M+H] Calcd.: 447.2; Found, 447.2.

### Step 3: 2-((2R,4S)-1-acryloyl-4-(4-amino-3-((4,6-difluoro-1,2-dimethyl-1H-benzo[d]imidazol-5-yl)ethynyl)-1H-pyrazolo[4,3-c]pyridin-1-yl)pyrrolidin-2-yl)acetonitrile formate

To a solution of 2-((2R,4S)-4-(4-amino-3-((4,6-difluoro-1,2-dimethyl-1H-benzo[d]imidazol-5-yl)ethynyl)-1H-pyrazolo[4,3-c]pyridin-1-yl)pyrrolidin-2-yl)acetonitrile 2,2,2-trifluoroacetate (57 mg, 0.12 mmol) and DIEA (77 mg, 0.60 mmol) in DCM (10 mL) at −50° C. under nitrogen atmosphere was added a solution of acryloyl chloride (10 mg, 0.11 mmol) in DCM (0.5 mL) slowly. The mixture was stirred at −50° C. for 0.5 h. The reaction mixture was quenched with water (15 mL) and extracted with DCM (10 mL) twice. The combined organic layers were dried over anhydrous sodium sulfate, filtered and concentrated in vacuo to give a residue which was purified by prep-HPLC to afford 2-((2R,4S)-1-acryloyl-4-(4-amino-3-((4,6-difluoro-1,2-dimethyl-1H-benzo[d]imidazol-5-yl)ethynyl)-1H-pyrazolo[4,3-c]pyridin-1-yl)pyrrolidin-2-yl)acetonitrile formate (17.8 mg, 28%) as a white solid. 1H NMR (400 MHz, DMSO-d6): 8.13 (s, 0.3H), 7.84 (d, J=6.4 Hz, 1H), 7.59 (d, J=9.2 Hz, 1H), 7.06 (d, J=6.4 Hz, 1H), 6.86-6.79 (m, 2H), 5.59 (dd, J=10.0, 16.4 Hz, 1H), 6.19 (dd, J=2.0, 16.8 Hz, 1H), 5.73-5.61 (m, 2H), 4.87-4.50 (m, 1H), 4.15-3.99 (m, 2H), 3.76 (s, 3H), 3.18-3.00 (m, 2H), 2.82-2.70 (m, 1H), 2.56 (s, 3H), 2.42-2.37 (m, 1H). [M+H] Calcd.: 501.3; Found, 501.3.

### Example 40: 1-((2R,4S)-4-(4-amino-3-((1-methyl-1H-indazol-5-yl)ethynyl)-1H-pyrazolo[3,4-d]pyrimidin-1-yl)-2-(methoxymethyl)pyrrolidin-1-yl)prop-2-en-1-one

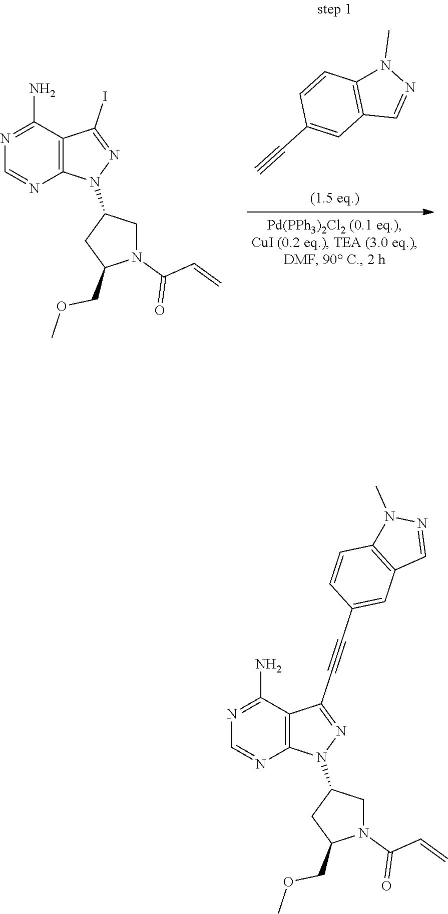 [SMILES: C=CC(=O)N1C[C@@H](n2nc(I)c3c(N)ncnc32)C[C@@H]1COC ; C#Cc1ccc2c(cnn2C)c1 ; C=CC(=O)N1C[C@@H](n2nc(C#Cc3ccc4c(cnn4C)c3)c3c(N)ncnc32)C[C@@H]1COC]

To a mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.1 g, 0.23 mmol) and 5-ethynyl-1-methylindazole (54.71 mg, 0.35 mmol) in DMF (3.00 mL) were added Pd(PPh3)2Cl2 (16.39 mg, 0.02 mmol), CuI (8.89 mg, 0.05 mmol) and TEA (70.89 mg, 0.701 mmol) at room temperature. under nitrogen atmosphere. The reaction mixture was degassed with argon for three times and stirred for 2 h at 90° C. The resulting mixture was concentrated under reduced pressure. The residue was purified by Prep-HPLC with the following conditions: Column: XBridge C18 OBD Prep Column, 10 μm, 19 mm×250 mm; Mobile Phase A: Water (10 mmol/L NH4HCO3), Mobile Phase B: ACN; Flow rate: 20 mL/min; Gradient: 40% B to 80% B in 5.8 min; Detector: UV 254 & 210 nm; RT: 5.55 min. The fractions that contained desired product were combined and concentrated to afford 1-[(2R,4S)-4-[4-amino-3-[2-(1-methylindazol-5-yl)ethynyl]pyrazolo[3,4-d]pyrimidin-1-yl]-2-(methoxymethyl)pyrrolidin-1-yl]prop-2-en-1-one (18.6 mg, 16%) as a white solid. MS ESI calculated for C24H24N8O2 [M+H]+, 457.20 found 457.20. 1H-NMR (300 MHz, CD3OD) δ 8.27 (s, 1H), 8.08 (d, J=13.1 Hz, 2H), 7.63 (d, J=3.6 Hz, 2H), 6.83-6.53 (m, 1H), 6.34-6.25 (m, 1H), 5.82-5.69 (m, 2H), 4.62 (d, J=10.6 Hz, 1H), 4.10-4.05 (m, 4H), 3.92-3.77 (m, 1H), 3.62-3.51 (m, 2H), 3.41 (d, J=1.5 Hz, 3H), 2.90-2.70 (m, 1H), 2.54-2.50 (m, 1H).

### Example 41: 1-[(2R,4S)-4-[4-amino-3-[2-(2-methylindazol-5-yl)ethynyl]pyrazolo[3,4-d]pyrimidin-1-yl]-2-(methoxymethyl)pyrrolidin-1-yl]prop-2-en-1-one

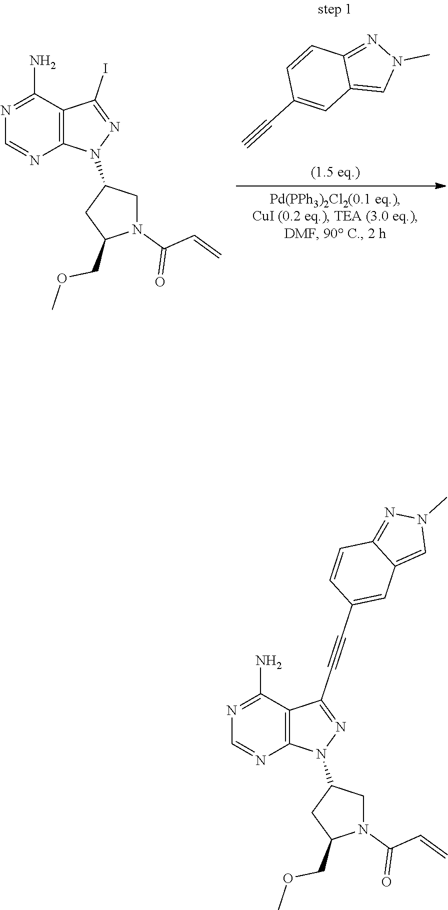

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (70.00 mg, 0.16 mmol) in DMF (2.00 mL) were added 5-ethynyl-2-methylindazole (38.30 mg, 0.25 mmol), Pd(PPh3)2Cl2 (11.47 mg, 0.02 mmol), CuI (6.23 mg, 0.03 mmol) and TEA (49.62 mg, 0.49 mmol) at room temperature. The reaction mixture was degassed with argon for three times and stirred for 2 h at 90° C. The mixture was allowed to cool down to room temperature. The resulting mixture was filtered and the filtrate was purified by Prep-HPLC with the following conditions Column: Atlantis Prep T3 OBD Column, 19*250 mm 10 u; Mobile Phase A: Water (0.1% FA), Mobile Phase B: ACN; Flow rate: 20 mL/min; Gradient: 30 B to 60 B in 6 min; 210/254 nm. The fractions that contained desired product were combined and concentrated to afford 1-[(2R,4S)-4-[4-amino-3-[2-(2-methylindazol-5-yl)ethynyl]pyrazolo[3,4-d]pyrimidin-1-yl]-2-(methoxymethyl)pyrrolidin-1-yl]prop-2-en-1-one (17.50 mg, 23%) as an off-white solid. MS ESI calculated for C24H24N8O2 [M+H]+, 457.21, found 457.10. H-NMR (300 MHz, d6-DMSO) δ 8.46 (s, 1H), 8.26-8.20 (m, 2H), 7.64 (d, J=8.9 Hz, 1H), 7.45 (d, J=8.9 Hz, 1H), 6.67-6.60 (m, 1H), 6.18-6.12 (m, 1H), 5.68-5.62 (m, 1H), 4.55-4.50 (m, 1H), 4.19 (s, 3H), 3.90-3.85 (m, 1H), 3.73-3.31 (m, 7H), 2.70-2.65 (m, 1H), 2.40-2.36 (m, 1H).

### Example 42: 1-[(2R,4S)-4-[4-amino-3-[2-(2-ethylindazol-5-yl)ethynyl]pyrazolo[3,4-d]pyrimidin-1-yl]-2-(methoxymethyl)pyrrolidin-1-yl]prop-2-en-1-one

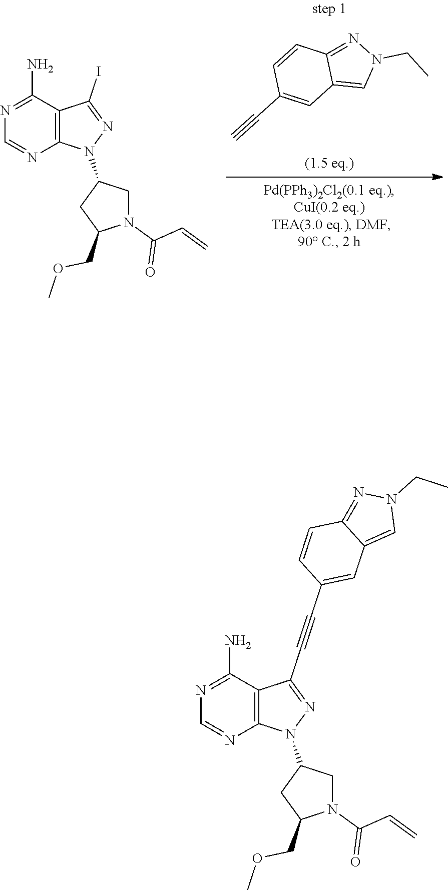

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (70.00 mg, 0.16 mmol) in DMF (2.00 mL) were added 2-ethyl-5-ethynylindazole (41.74 mg, 0.25 mmol), Pd(PPh3)2Cl2 (11.47 mg, 0.02 mmol), CuI (6.23 mg, 0.03 mmol) and TEA (49.62 mg, 0.49 mmol) at room temperature. The reaction mixture was degassed with argon for three times and stirred for 2 h at 90° C. The resulting mixture was concentrated under reduced pressure. The residue was purified by reverse flash chromatography with the following conditions: column, C18 silica gel; mobile phase, NH4HCO3 in water, 0% to 33% gradient in 10 min; detector, UV 254 nm to afford crude product as a brown solid. The crude product (168.00 mg) was purified by Prep-HPLC with the following conditions Column: Atlantis Prep T3 OBD Column, 19*250 mm 10 u; Mobile Phase A Water (0.1% FA), Mobile Phase B: ACN; Flow rate: 20 mL/min; Gradient: 40 B to 65 B in 6 min; 210/254 nm; RT1:5.95. The fractions that contained desired product were combined and concentrated to afford 1-[(2R,4S)-4-[4-amino-3-[2-(2-ethylindazol-5-yl)ethynyl]pyrazolo[3,4-d]pyrimidin-1-yl]-2-(methoxymethyl)pyrrolidin-1-yl]prop-2-en-1-one (28.10 mg, 36%) as an off-white solid. MS ESI calculated for C25H26N8O2 [M+H]+, 471.23, found 471.10. H-NMR (300 MHz, d6-DMSO) δ 8.50 (s, 1H), 8.25-8.21 (m, 2H), 7.66 (d, J=8.9 Hz, 1H), 7.46 (dd, J=8.9, 1.6 Hz, 1H), 6.67-6.62 (m, 1H), 6.19-6.12 (m, 1H), 5.75-5.55 (m, 2H), 4.50-4.40 (m, 3H), 4.17-3.74 (m, 2H), 3.71-3.45 (m, 2H), 3.30 (s, 3H), 2.75-2.55 (m, 1H), 2.39-2.35 (m, 1H), 1.51 (t, J=7.2 Hz, 3H).

### Example 43: 2-((2R,4S)-1-acryloyl-4-(4-amino-3-((1,2-dimethyl-1H-benzo[d]imidazol-5-yl)ethynyl)-1H-pyrazolo[4,3-c]pyridin-1-yl)pyrrolidin-2-yl)acetonitrile

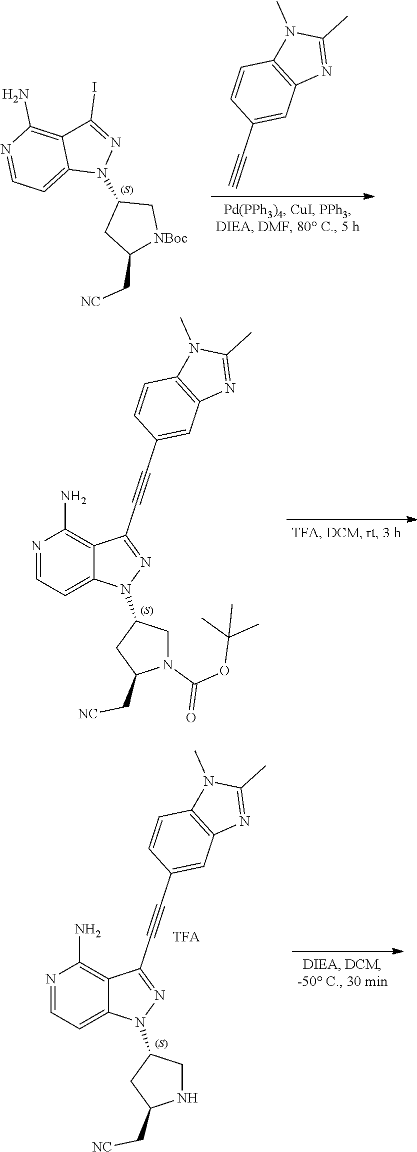

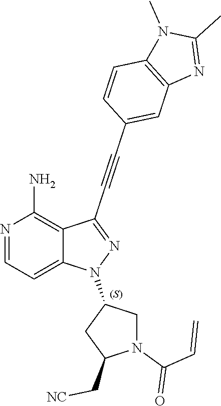

### Step 1: (2R,4S)-tert-butyl 4-(4-amino-3-((1,2-dimethyl-1H-benzo[d]imidazol-5-yl)ethynyl)-1H-pyrazolo[4,3-c]pyridin-1-yl)-2-(cyanomethyl)pyrrolidine-1-carboxylate

To a mixture of (2R,4S)-tert-butyl 4-(4-amino-3-iodo-1H-pyrazolo[4,3-c]pyridin-1-yl)-2-(cyanomethyl)pyrrolidine-1-carboxylate (200 mg, 0.42 mmol), 5-ethynyl-1,2-dimethyl-1H-benzo[d]imidazole (87 mg, 0.51 mmol) and CuI (11 mg, 0.06 mmol) in DMF (10 mL) was added Pd(PPh3)4 (242 mg, 0.21 mmol), PPh3 (6 mg, 0.02 mmol) and DIEA (163 mg, 1.26 mmol) under nitrogen atmosphere and stirred at 80° C. for 5 h. After cooling down to room temperature, the reaction mixture was diluted with water (20 mL) and extracted with DCM (20 mL) for three times, the combined organic layers were dried over anhydrous sodium sulfate, filtered and concentrated in vacuo to give a residue, which was purified by flash (DCM/MeOH=20/1) to afford (2R,4S)-tert-butyl 4-(4-amino-3-((1,2-dimethyl-1H-benzo[d]imidazol-5-yl)ethynyl)-1H-pyrazolo[4,3-c]pyridin-1-yl)-2-(cyanomethyl)pyrrolidine-1-carboxylate (80 mg, 36%) as a yellow solid. [M+H] Calcd.: 511.3; Found, 511.3.

### Step 2: 2-((2R,4S)-4-(4-amino-3-((1,2-dimethyl-1H-benzo[d]imidazol-5-yl)ethynyl)-1H-pyrazolo[4,3-c]pyridin-1-yl)pyrrolidin-2-yl)acetonitrile 2,2,2-trifluoroacetate

To a solution of (2R,4S)-tert-butyl 4-(4-amino-3-((1,2-dimethyl-1H-benzo[d]imidazol-5-yl)ethynyl)-1H-pyrazolo[4,3-c]pyridin-1-yl)-2-(cyanomethyl)pyrrolidine-1-carboxylate (80 mg, 0.16 mmol) in DCM (10 mL) was added TFA (0.5 mL). The reaction mixture was stirred at rt for 3 h. The reaction mixture was concentrated in vacuum to give 2-((2R,4S)-4-(4-amino-3-((1,2-dimethyl-1H-benzo[d]imidazol-5-yl)ethynyl)-1H-pyrazolo[4,3-c]pyridin-1-yl)pyrrolidin-2-yl)acetonitrile 2,2,2-trifluoroacetate (64 mg, crude) as a yellow solid. [M+H] Calcd.: 411.2; Found, 411.2.

### Step 3: 2-((2R,4S)-1-acryloyl-4-(4-amino-3-((1,2-dimethyl-1H-benzo[d]imidazol-5-yl)ethynyl)-1H-pyrazolo[4,3-c]pyridin-1-yl)pyrrolidin-2-yl)acetonitrile

To a solution of 2-((2R,4S)-4-(4-amino-3-((1,2-dimethyl-1H-benzo[d]imidazol-5-yl)ethynyl)-1H-pyrazolo[4,3-c]pyridin-1-yl)pyrrolidin-2-yl)acetonitrile 2,2,2-trifluoroacetate (40 mg, 0.09 mmol) and DIEA (58 mg, 0.45 mmol) in DCM (10 mL) at −50° C. under nitrogen atmosphere was added a solution of acryloyl chloride (8 mg, 0.08 mmol) in DCM (0.5 mL) slowly. The mixture was stirred at −50° C. for 0.5 h. The reaction mixture was quenched with water (15 mL) and extracted with DCM (10 mL) twice. The combined organic layers were dried over anhydrous sodium sulfate, filtered and concentrated in vacuo to give a residue which was purified by prep-HPLC to afford 2-((2R,4S)-1-acryloyl-4-(4-amino-3-((1,2-dimethyl-1H-benzo[d]imidazol-5-yl)ethynyl)-1H-pyrazolo[4,3-c]pyridin-1-yl)pyrrolidin-2-yl)acetonitrile (7 mg, 15%) as a white solid. 1H NMR (400 MHz, DMSO-d6): 7.85 (s, 1H), 7.81 (d, J=6.0 Hz, 1H), 7.58 (d, J=8.4 Hz, 1H), 7.47 (d, J=8.0 Hz, 1H), 6.96 (d, J=6.4 Hz, 1H), 6.59 (dd, J=10.0, 16.8 Hz, 1H), 6.39 (br, 2H), 6.19 (dd, J=2.0, 16.8 Hz, 1H), 5.71 (dd, J=1.6, 6.4 Hz, 1H), 5.59-5.57 (m, 1H), 4.87-4.49 (m, 1H), 4.13 (dd, J=6.4, 11.2 Hz, 1H), 3.97 (dd, J=4.0, 10.8 Hz, 1H), 3.76 (s, 3H), 3.19-2.97 (m, 2H), 2.73-2.67 (m, 1H), 2.55 (s, 3H), 2.40-2.33 (m, 1H). [M+H] Calcd.: 465.2; Found, 465.2.

### Example 44: 1-((2R,4S)-4-(4-amino-3-((4,6-difluoro-2-methyl-2H-indazol-5-yl)ethynyl)-1H-pyrazolo[3,4-d]pyrimidin-1-yl)-2-(methoxymethyl)pyrrolidin-1-yl)prop-2-en-1-one

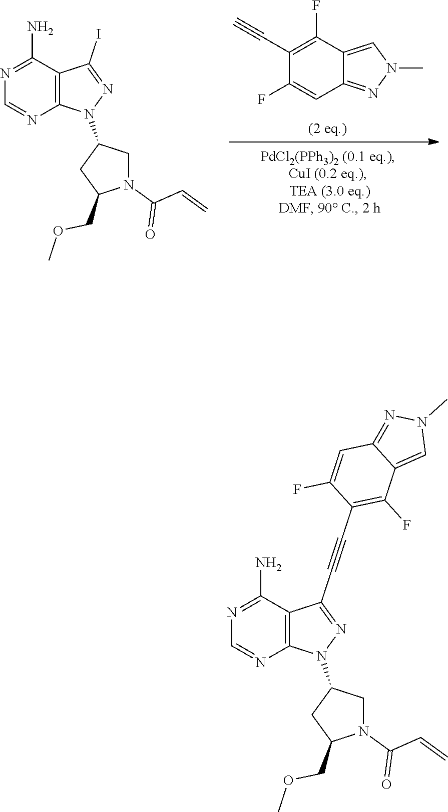 [SMILES: C=CC(=O)N1C[C@@H](n2nc(I)c3c(N)ncnc32)C[C@@H]1COC ; C#Cc1c(F)cc2nn(C)cc2c1F ; C=CC(=O)N1C[C@@H](n2nc(C#Cc3c(F)cc4nn(C)cc4c3F)c3c(N)ncnc32)C[C@@H]1COC]

To a stirred mixture of 1-((2R,4S)-4-(4-amino-3-iodo-1H-pyrazolo[3,4-d]pyrimidin-1-yl)-2-(methoxymethyl)pyrrolidin-1-yl)prop-2-en-1-one (0.10 g, 0.23 mmol), 5-ethynyl-4,6-difluoro-2-methyl-2H-indazole (89.75 mg, 0.47 mmol), Pd(PPh3)2Cl2 (16.39 mg, 0.023 mmol) and CuI (8.89 mg, 0.05 mmol) in DMF was added TEA (70.89 mg, 0.70 mmol). The reaction mixture was degassed with argon for three times and stirred for 2 h at 90° C. The resulting mixture was purified by Prep-HPLC with the following conditions: Column: SunFire Prep C18 OBD Column, 19×150 mm 5 um 10 nm; Mobile Phase A: Water (0.1% FA), Mobile Phase B: ACN; Flow rate: 20 mL/min; Gradient: 35 B to 60 B in 4.3 min; 254/210 nm; RT: 4.35. The fractions contained desired product were combined and concentrated to afford 1-((2R,4S)-4-(4-amino-3-((4,6-difluoro-2-methyl-2H-indazol-5-yl)ethynyl)-1H-pyrazolo[3,4-d]pyrimidin-1-yl)-2-(methoxymethyl)pyrrolidin-1-yl)prop-2-en-1-one (43.4 mg, 38%) as a white solid. ESI calculated for C₂₄H₂₂F₂N₈O₂ [M+H]+, 493.18; found 493.20. H-NMR (400 MHz, DMSO-d6) δ 8.31 (d, J=2.3 Hz, 1H), 7.45 (dd, J=9.7, 1.9 Hz, 1H), 7.10-7.16 (m, 1H), 6.74-6.53 (m, 1H), 6.12-6.18 (m, 1H), 5.77-5.50 (m, 2H), 4.54 (d, J=57.2 Hz, 1H), 4.27 (s, 3H), 4.15-3.80 (m, 2H), 3.66-3.45 (m, 2H), 3.33 (d, J=5.8 Hz, 3H), 2.77-2.57 (m, 1H), 2.42 (d, J=3.6 Hz, 1H).

### Example 45: 1-((2R,4S)-4-(4-amino-3-((4,6-difluoro-2-methyl-2H-indazol-5-yl)ethynyl)-1H-pyrazolo[4,3-c]pyridin-1-yl)-2-(methoxymethyl)pyrrolidin-1-yl)prop-2-en-1-one

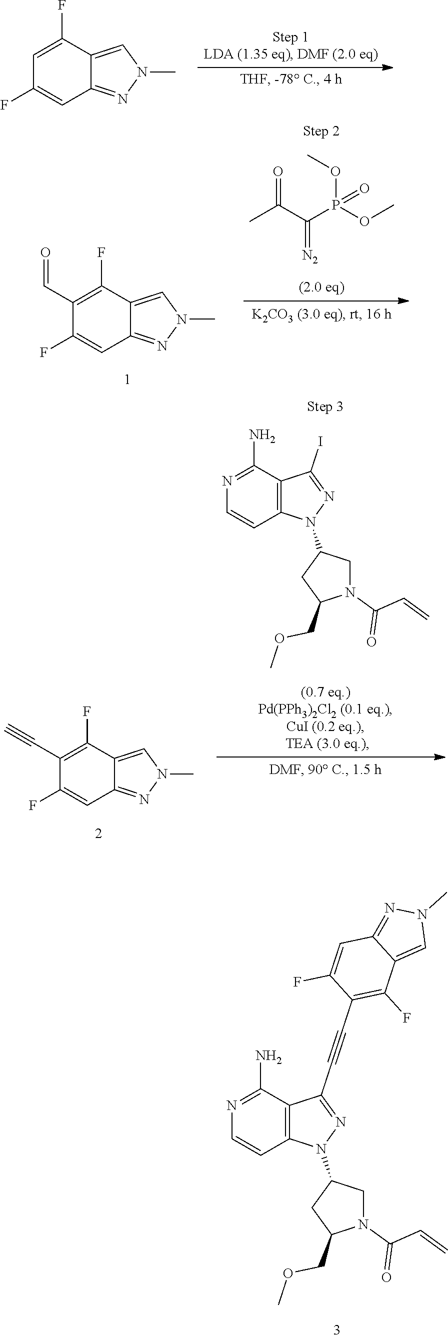

### Step 1: 4,6-difluoro-2-methyl-2H-indazole-5-carbaldehyde

To a stirred solution of LDA (7.03 mL, 14.06 mmol) in THF (70.00 mL) was added 4,6-difluoro-2-methylindazole (1.75 g, 10.41 mmol) in THF (15.00 mL) dropwise at −78° C. under argon atmosphere. The reaction mixture was stirred for 2 h at −78° C. To the above mixture was added DMF (1.61 mL, 20.81 mmol) dropwise at −78° C. The resulting mixture was stirred for additional 2 h. The resulting mixture was quenched with sat. NH₄Cl (aq.) at 0° C., extracted with EtOAc (2×500 mL). The combined organic layers were washed with water (2×200 mL), dried over anhydrous Na₂SO₄ and filtered. The filtrate was concentrated under reduced pressure. The residue was purified by silica gel column chromatography, eluted with PE/EtOAc (3:2). The fractions contained desired product were combined and concentrated to afford 4,6-difluoro-2-methyl-2H-indazole-5-carbaldehyde (1.09 g, 53%) as an off-white solid. H NMR (300 MHz, Chloroform-d) δ 10.34 (s, 1H), 7.3-7.26 (m, 1H), 6.98-6.85 (m, 1H), 4.52 (d, J=1.2 Hz, 3H).

### Step 2: 5-ethynyl-4,6-difluoro-2-methyl-2H-indazole

To a stirred mixture of 4,6-difluoro-2-methyl-2H-indazole-5-carbaldehyde (1.09 g, 5.56 mmol) and K₂CO₃ (2.30 g, 16.64 mmol) in MeOH (30.00 mL) was added dimethyl (1-diazo-2-oxopropyl)phosphonate (1.67 mL, 11.13 mmol) dropwise at room temperature under argon atmosphere. The reaction mixture was stirred for overnight. The resulting mixture was diluted with water (200 mL), extracted with EtOAc (3×200 mL). The combined organic layers were washed with brine (2×100 mL), dried over anhydrous Na₂SO₄ and filtered. The filtrate was concentrated under reduced pressure. The residue was purified by silica gel column chromatography, eluted with PE/EtOAc (3:2). The fractions contained desired product were combined and concentrated to afford 5-ethynyl-4,6-difluoro-2-methylindazole (0.88 g, 82%) as an off-white solid. H NMR (400 MHz, Chloroform-d) δ 7.14 (dd, J=9.2, 2.0 Hz, 1H), 6.57-6.68 (m, 1H), 4.24 (s, 3H), 3.92 (s, 1H).

### Step 3: 1-((2R,4S)-4-(4-amino-3-((4,6-difluoro-2-methyl-2H-indazol-5-yl)ethynyl)-1H-pyrazolo[4,3-c]pyridin-1-yl)-2-(methoxymethyl)pyrrolidin-1-yl)prop-2-en-1-one

To a mixture of 1-((2R,4S)-4-(4-amino-3-iodo-1H-pyrazolo[4,3-c]pyridin-1-yl)-2-(methoxymethyl)pyrrolidin-1-yl)prop-2-en-1-one (0.10 mg, 0.23 mmol), 5-ethynyl-4,6-difluoro-2-methyl-2H-indazole (90.0 mg, 0.47 mmol), Pd(PPh₃)₂Cl₂ (16.4 mg, 0.023 mmol) and CuI (8.92 mg, 0.047 mmol) in DMF (1 mL) was added TEA (71.05 mg, 0.70 mmol). The reaction mixture was degassed with argon for three times and stirred for 2 h at 90° C. The resulting mixture was purified by Prep-HPLC with the following conditions: Column: Xselect CSH F-Phenyl OBD Column 19*150 mm 5 um; Mobile Phase A: Water (0.1% FA), Mobile Phase B: MeOH-HPLC; Flow rate: 20 mL/min; Gradient: 35 B to 65 B in 5.3 min; 254/210 nm; RT: 5.3. The fractions contained desired product were combined and concentrated to afford 1-((2R,4S)-4-(4-amino-3-((4,6-difluoro-2-methyl-2H-indazol-5-yl)ethynyl)-1H-pyrazolo[4,3-c]pyridin-1-yl)-2-(methoxymethyl)pyrrolidin-1-yl)prop-2-en-1-one (68.8 mg, 55%) as a white solid. ESI calculated for C₂₅H₂₃F₂N₇O₂ [M+H]⁺, 492.19; found 492.20. H-NMR (300 MHz, DMSO-d₆) δ 7.86 (d, J=6.1 Hz, 1H), 7.46 (d, J=9.4 Hz, 1H), 7.12-7.16 (m, 1H), 7.03 (d, J=6.1 Hz, 1H), 6.84-6.39 (m, 3H), 6.18 (d, J=16.6 Hz, 1H), 5.70 (t, J=10.9 Hz, 1H), 5.55 (s, 1H), 4.55 (d, J=39.8 Hz, 1H), 4.29 (d, J=1.1 Hz, 3H), 4.15-3.76 (m, 2H), 3.67-3.49 (m, 2H), 3.34 (s, 3H), 2.69-2.75 (m, 1H), 2.42-2.40 (m, 1H).

### Example 46: 1-((2R,4S)-4-(4-amino-3-((1-methyl-2-(trifluoromethyl)-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

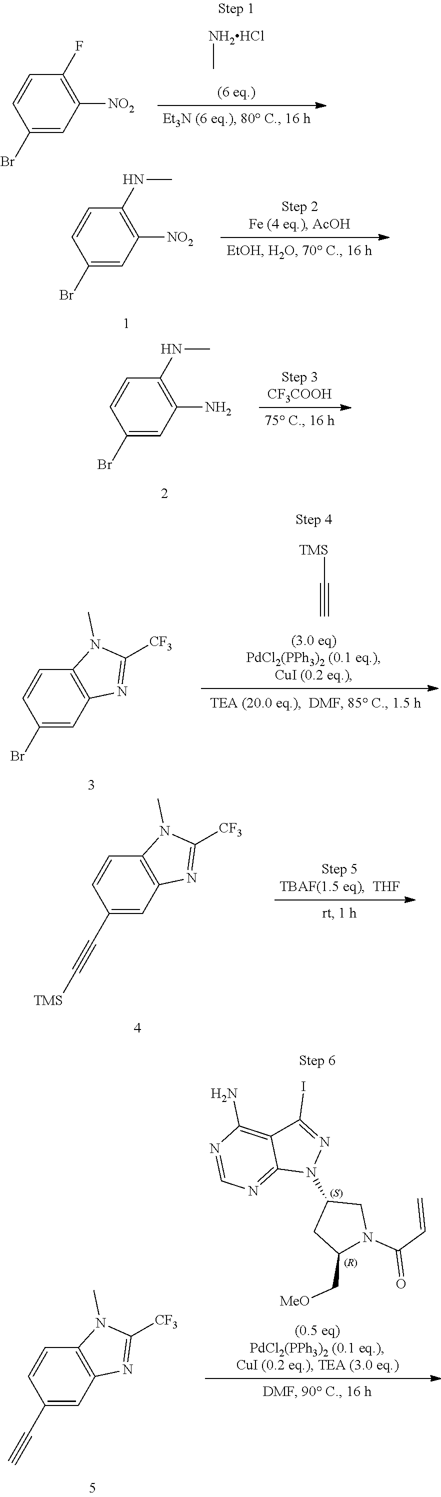

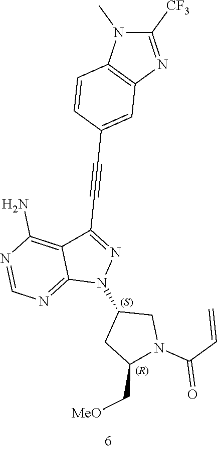

### Step 1: 4-iodo-N-methyl-2-nitroaniline

To a mixture of 1-fluoro-4-iodo-2-nitrobenzene (10.00 g, 37.45 mmol) in TEA (26.53 g, 262.18 mmol) was added methylamine (5.82 g, 187.27 mmol). The reaction mixture was stirred for overnight at 80° C. under nitrogen atmosphere. The resulting mixture was diluted with water (200 mL), extracted with EtOAc (3×200 mL). The combined organic layers were washed with brine (400 mL), dried over anhydrous Na₂SO₄ and filtered. The filtrate was concentrated under reduced pressure. This resulted in 4-iodo-N-methyl-2-nitroaniline (10.5 g, 96%) as a yellow solid which was used in the next step without further purification. MS ESI calculated for C₇H₇BrN₂O₂ [M+H]⁺, 248.98, found 249.00.

### Step 2: 4-iodo-N1-methylbenzene-1,2-diamine

To a mixture of 4-iodo-N-methyl-2-nitroaniline (5.00 g, 17.98 mmol) and NH4Cl (4.81 g, 89.91 mmol) in EtOH and water a was added Fe (4.02 g, 71.93 mmol). The reaction mixture was stirred for overnight at 70° C. under nitrogen atmosphere. The resulting mixture was filtered, the filter cake was washed with EtOH (3×200 mL). The filtrate was concentrated under reduced pressure. The residue was diluted with water (200 mL), extracted with EtOAc (3×200 mL). The combined organic layers were washed with brine (400 mL), dried over anhydrous Na2SO4 and filtered. The filtrate was concentrated under reduced pressure and dried to offord 4-iodo-N1-methylbenzene-1,2-diamine (4.5 g, 98%) as a black oil which was used in the next step without further purification. MS ESI calculated for C7H9BrN2 [M+H]+, 200.99; found 201.00.

### Step 3: 5-bromo-1-methyl-2-(trifluoromethyl)-1H-benzo[d]imidazole

A solution of 4-bromo-N1-methylbenzene-1,2-diamine (1.00 g, 4.97 mmol) and TFA (5.00 mL) was stirred for 16 h at 75° C. under nitrogen atmosphere. The resulting mixture was concentrated under reduced pressure. The residue was quenched by the addition of sat. NaHCO₃(aq.) (30 mL), the resulting mixture was extracted with EtOAc (3×50 mL). The organic layers were combined, dried over anhydrous Na2SO4 and filtered. The filtrate was concentrated under reduced pressure. The residue was purified by silica gel column chromatography, eluted with PE/EtOAc (6:1). The fractions contained desired product were combined and concentrated to afford 5-bromo-1-methyl-2-(trifluoromethyl)-1H-benzo[d]imidazole (0.90 g, 65%) as a light brown solid. MS ESI calculated for C9H6BrF3N2 [M+H]+, 278.97; found 279.00.

### Step 4: 1-methyl-2-(trifluoromethyl)-5-((trimethylsilyl)ethynyl)-1H-benzo[d]imidazole

To a stirred mixture of 5-bromo-1-methyl-2-(trifluoromethyl)-1H-benzo[d]imidazole (2.50 g, 7.67 mmol), CuI (0.29 g, 1.53 mmol), Pd(PPh3)2Cl2 (0.54 g, 0.77 mmol) in DMF (25.00 mL, 342.01 mmol) were added trimethylsilylacetylene (3.25 mL, 33.10 mmol) and TEA (21.31 mL, 210.64 mmol). T The reaction mixture was degassed with argon for three times and stirred for 1.5 h at 85° C. The resulting mixture was diluted with water (150 mL) and extracted with EA (3×150 mL). The combined organic layers were washed with brine (2×200 mL), dried over anhydrous Na2SO4, filtered and concentrated under reduced pressure. The residue was purified by silica gel column chromatography, eluted with 14% EA in PE. The fractions contained desired product were combined and concentrated to afford 1-methyl-2-(trifluoromethyl)-5-((trimethylsilyl)ethynyl)-1H-benzo[d]imidazole (2.1 g, 83%) as a brown yellow solid. MS ESI calculated for C14H15F3N2Si [M+H]+, 297.10, found 297.10.

### Step 5: 5-ethynyl-1-methyl-2-(trifluoromethyl)-1H-benzo[d]imidazole

To a stirred solution of 1-methyl-2-(trifluoromethyl)-5-((trimethylsilyl)ethynyl)-1H-benzo[d]imidazole (2.10 g, 7.09 mmol) in THF (21.00 mL) was added TBAF (10.63 mL) dropwise at 0° C. under nitrogen atmosphere. The reaction mixture was stirred for 1 h at room temperature. The resulting mixture was diluted with water (80 mL), extracted with EtOAc (3×100 mL). The combined organic layers were washed with brine (150 mL), dried over anhydrous Na2SO4 and filtered. The filtrate was concentrated under reduced pressure. The residue was purified by silica gel column chromatography, eluted with PE/EtOAc (5:1). The fractions contained desired product were combined and concentrated to afford 5-ethynyl-1-methyl-2-(trifluoromethyl)-1H-benzo[d]imidazole (1.48 g, 84%) as an off-white solid. MS ESI calculated for C11H7F3N2 [M+H]+, 225.06, found 225.05.

### Step 6: 1-((2R,4S)-4-(4-amino-3-((1-methyl-2-(trifluoromethyl)-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

To a stirred mixture of 1-((2R,4S)-4-(4-amino-3-iodo-1H-pyrazolo[3,4-d]pyrimidin-1-yl)-2-(methoxymethyl)pyrrolidin-1-yl)prop-2-en-1-one (0.10 g, 0.16 mmol), 5-ethynyl-1-methyl-2-(trifluoromethyl)-1H-benzo[d]imidazole (89.6 mg, 0.33 mmol), Pd(PPh₃)₂Cl₂ (16.4 mg, 0.02 mmol) and CuI (8.9 mg, 0.03 mmol) in DMF (1 mL) was added TEA (52.5 mg, 0.49 mmol). The reaction mixture was degassed with argon for three times and stirred for 2 h at 90° C. The resulting mixture was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 50 B to 70 B in 4.3 min; 210/254 nm; RT: 4.02. The fractions contained desired product were combined and concentrated to afford 1-((2R,4S)-4-(4-amino-3-((1-methyl-2-(trifluoromethyl)-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one (31.5 mg, 37%) as a white solid. ESI calculated for C₂₅H₂₃F₃N₈O₂ [M+H]⁺, 525.19; found 525.10. H-NMR (400 MHz, DMSO-d₆) δ 8.28 (d, J=2.7 Hz, 2H), 7.90 (d, J=8.6 Hz, 1H), 7.83 (dd, J=8.5, 1.5 Hz, 1H), 6.52-6.83 (m, 1H), 6.12-6.25 (m, 1H), 5.76-5.55 (m, 2H), 4.70-4.40 (m, 1H), 4.12-4.08 (dd, J=10.7, 7.5 Hz, 1H), 4.02 (d, J=1.2 Hz, 3H), 3.97-3.92 (m, 1H), 3.86-3.3.79 (m, 1H), 3.68-3.60 (m, 1H), 3.55-3.45 (m, 1H), 3.34 (s, 3H), 2.76-2.57 (m, 1H), 2.46-2.32 (m, 1H).

### Example 47: 1-((2R,4S)-4-(4-amino-3-((1-methyl-2-(trifluoromethyl)-1H-benzo[d]imidazol-5-yl)ethynyl)-1H-pyrazolo[4,3-c]pyridin-1-yl)-2-(methoxymethyl)pyrrolidin-1-yl)prop-2-en-1-one

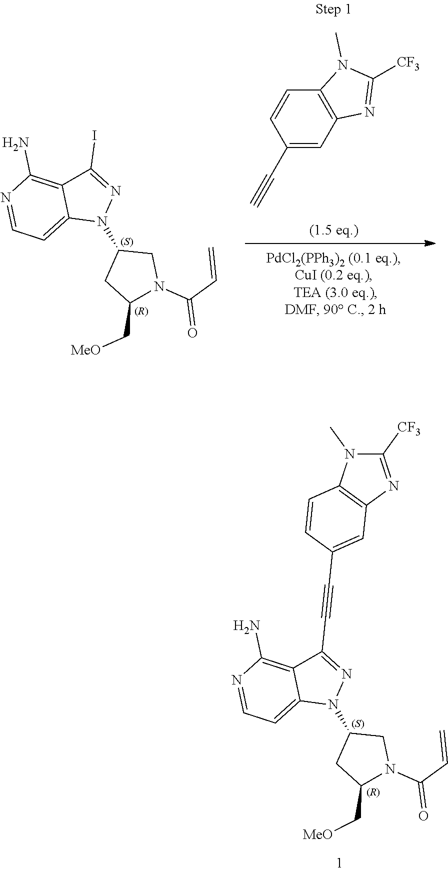

To a stirred mixture of 1-((2R,4S)-4-(4-amino-3-iodo-1H-pyrazolo[4,3-c]pyridin-1-yl)-2-(methoxymethyl)pyrrolidin-1-yl)prop-2-en-1-one (0.10 g, 0.16 mmol), 5-ethynyl-1-methyl-2-(trifluoromethyl)-1H-benzo[d]imidazole (89.2 mg, 0.34 mmol), Pd(PPh₃)₂Cl₂ (16.5 mg, 0.02 mmol) and CuI (11.5 mg, 0.03 mmol) in DMF (1 mL) was added TEA (50.11 mg, 0.50 mmol). The reaction mixture was degassed with argon for three times and stirred for 2 h at 90° C. The resulting mixture was purified by Prep-HPLC with the following conditions Column: Atlantis Prep T3 OBD Column, 19*250 mm 10 u; Mobile Phase A: Water (0.1% FA), Mobile Phase B: ACN; Flow rate: 20 mL/min; Gradient: 30 B to 60 B in 6 min; 210/254 nm; RT: 5.58. The fractions contained desired product were combined and concentrated to afford 1-((2R,4S)-4-(4-amino-3-((1-methyl-2-(trifluoromethyl)-1H-benzo[d]imidazol-5-yl)ethynyl)-1H-pyrazolo[4,3-c]pyridin-1-yl)-2-(methoxymethyl)pyrrolidin-1-yl)prop-2-en-1-one (48.4 mg, 56%) as a white solid. ESI calculated for C₂₆H₂₄F₃N₇O₂ [M+H]⁺, 524.19; found 524.05. H-NMR (300 MHz, DMSO-d6) δ 8.20 (d, J=24.5 Hz, 1H), 7.93 (d, J=8.6 Hz, 1H), 7.83-7.79 (m, 2H), 7.08-6.91 (m, 1H), 6.86-6.50 (m, 1H), 6.45 (s, 1H), 6.18 (d, J=16.4 Hz, 2H), 5.71 (t, J=11.3 Hz, 1H), 5.51 (s, 1H), 4.56 (d, J=40.7 Hz, 1H), 4.30-3.97 (m, 4H), 3.89-3.85 (m, 1H), 3.72-3.44 (m, 2H), 3.35 (d, J=3.3 Hz, 3H), 2.65-2.64 (m, 1H), 2.49-2.45 (m, 1H).

### Example 48: 1-((2R,4S)-4-(4-amino-3-((4,6-difluoro-1-methyl-1H-indazol-5-yl)ethynyl)-1H-pyrazolo[3,4-d]pyrimidin-1-yl)-2-(methoxymethyl)pyrrolidin-1-yl)prop-2-en-1-one

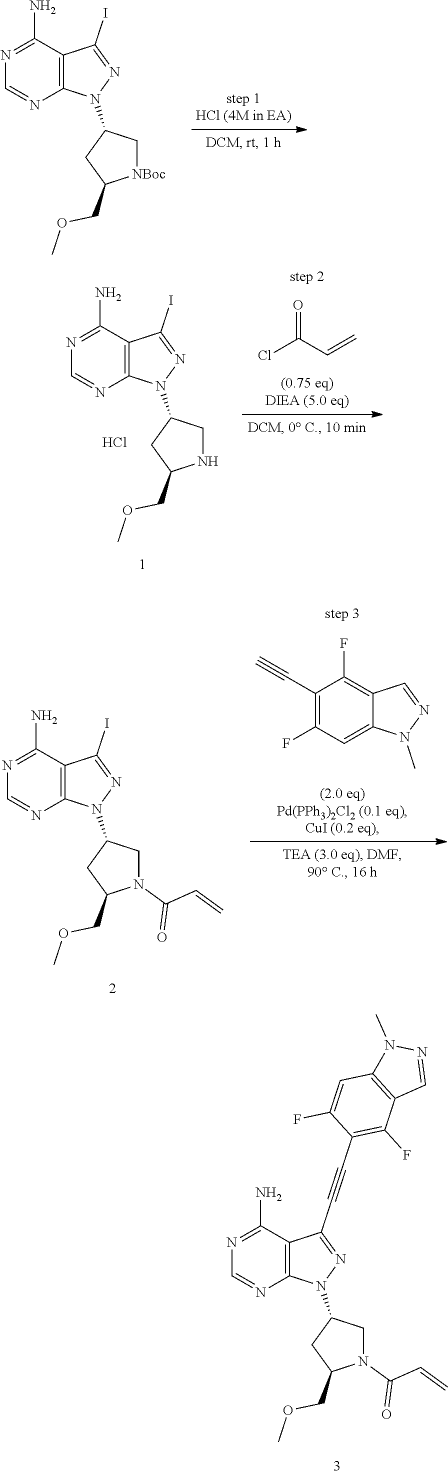

### Step 1: 3-iodo-1-[(5R)-5-(methoxymethyl)pyrrolidin-3-yl]pyrazolo[3,4-d]pyrimidin-4-amine hydrochloride

To a stirred solution of tert-butyl (2R)-4-[4-amino-3-iodopyrazolo[3,4-d]pyrimidin-1-yl]-2-(methoxymethyl)pyrrolidine-1-carboxylate (5.40 g, 11.39 mmol) in DCM (50.00 mL) was added HCl in EA (4M) (50.00 mL) dropwise at 0° C. under argon atmosphere. The resulting mixture was stirred for 1 h at room temperature. The precipitated solids were collected by filtration and washed with DCM (3×30 mL). The residue was dried under reduced pressure to afford 3-iodo-1-[(5R)-5-(methoxymethyl)pyrrolidin-3-yl]pyrazolo[3,4-d]pyrimidin-4-amine hydrochloride (4.8 g, 92%) as a light yellow solid. MS ESI calculated for C₁₁H₁₆ClIN₆O [M+H−HCl]⁺, 375.04, found 375.10.

### Step 2: 1-[(2R)-4-[4-amino-3-iodopyrazolo[3,4-d]pyrimidin-1-yl]-2-(methoxymethyl)pyrrolidin-1-yl]prop-2-en-1-one

To a stirred mixture of 3-iodo-1-[(5R)-5-(methoxymethyl)pyrrolidin-3-yl]pyrazolo[3,4-d]pyrimidin-4-amine hydrochloride (0.57 g, 1.39 mmol) and DIEA (1.21 mL, 6.95 mmol) in DCM (19.00 mL) was added acryloyl chloride (4.16 mL, 1.04 mmol) (0.25 M in DCM) dropwise at 0° C. under argon atmosphere. The resulting mixture was stirred for 10 mine. The resulting mixture was concentrated under reduced pressure. The residue was purified by silica gel column chromatography, eluted with CH₂Cl₂/MeOH (10:1). The fractions contained desired product were combined and concentrated to afford 1-[(2R)-4-[4-amino-3-iodopyrazolo[3,4-d]pyrimidin-1-yl]-2-(methoxymethyl)pyrrolidin-1-yl]prop-2-en-1-one (0.41 g, 68%) as an off-white solid. MS ESI calculated for C₁₄H₁₇IN₆O₂ [M+H]⁺, 429.05, found 428.95.

### Step 3: 1-[(2R,4S)-4-[4-amino-3-[2-(4,6-difluoro-1-methylindazol-5-yl)ethynyl]pyrazolo[3,4-d]pyrimidin-1-yl]-2-(methoxymethyl)pyrrolidin-1-yl]prop-2-en-1-one)

To a mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.15 g, 0.35 mmol), 5-ethynyl-4,6-difluoro-1-methylindazole (0.13 g, 0.70 mmol), CuI (13.34 mg, 0.07 mmol) and Pd(PPh₃)₂Cl₂ (24.59 mg, 0.04 mmol) in DMF (2.00 mL) was added TEA (0.15 mL, 1.48 mmol). The reaction mixture was degassed with argon for three times and stirred for 16 h at 90° C. The resulting mixture was duilted with water (50 mL), extracted with EtOAc (3×50 mL). The combined organic layers were washed with brine (2×30 mL), dried over anhydrous Na₂SO₄ and filtered. The filtrate was concentrated under reduced pressure. The residue was purified by reverse phase flash with the following conditions Column: SunFire Prep C18 OBD Column, 19×150 mm 5 um 10 nm; Mobile Phase A: Water (0.1% FA), Mobile Phase B: ACN; Flow rate: 20 mL/min; Gradient: 20 B to 50 B in 4.3 min; 210/254 nm; RT1:4.02. The fractions contained desired product were combined and concentrated to afford 1-[(2R,4S)-4-[4-amino-3-[2-(4,6-difluoro-1-methylindazol-5-yl)ethynyl]pyrazolo[3,4-d]pyrimidin-1-yl]-2-(methoxymethyl)pyrrolidin-1-yl]prop-2-en-1-one (65.1 mg, 38%) as an off-white solid. MS ESI calculated for C₂₄H₂₂F₂N₈O₂ [M+H]⁺, 493.18, found 493.20. H NMR (400 MHz, DMSO-d₆) δ 8.37-8.28 (m, 2H), 7.74 (d, J=9.6 Hz, 1H), 6.69-6.65 (m, 1H), 6.19-6.15 (m 1H), 5.75-5.58 (m, 2H), 4.54 (d, J=54.4 Hz, 1H), 4.07 (s, 3H), 4.02-3.92 (m, 1H), 3.84-3.78 (m, 1H), 3.67-3.45 (m, 2H), 3.32 (s, 3H), 2.74-2.55 (m, 1H), 2.44-2.36 (m, 1H).

### Example 49: 1-((2R,4S)-4-(4-amino-3-((2-ethyl-4,6-difluoro-2H-indazol-5-yl)ethynyl)-1H-pyrazolo[3,4-d]pyrimidin-1-yl)-2-(methoxymethyl)pyrrolidin-1-yl)prop-2-en-1-one

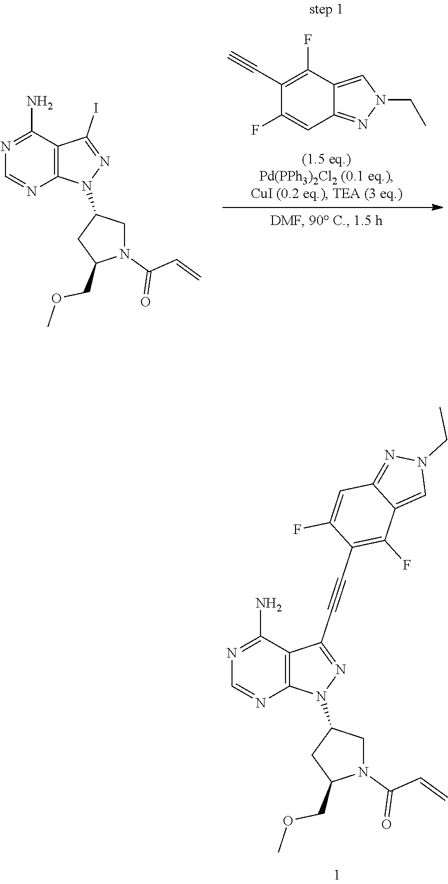 [SMILES: C=CC(=O)N1C[C@@H](n2nc(I)c3c(N)ncnc32)C[C@@H]1COC ; C#Cc1c(F)cc2nn(CC)cc2c1F ; C=CC(=O)N1C[C@@H](n2nc(C#Cc3c(F)cc4nn(CC)cc4c3F)c3c(N)ncnc32)C[C@@H]1COC]

To a stirred solution of 1-[(2R,4S)-4-[4-amino-3-iodopyrazolo[3,4-d]pyrimidin-1-yl]-2-(methoxymethyl)pyrrolidin-1-yl]prop-2-en-1-one (0.13 g, 0.30 mmol), 2-ethyl-5-ethynyl-4,6-difluoroindazole (93.89 mg, 0.45 mmol), Pd(PPh₃)₂Cl₂ (21.31 mg, 0.03 mmol) and CuI (11.56 mg, 0.06 mmol) in DMF (2.00 mL) was added TEA (92.16 mg, 0.91 mmol). The reaction mixture was degassed with argon for three times and stirred for 1.5 h at 90° C. The residue was purified by reverse flash chromatography with the following conditions: column, C18 silica gel; mobile phase ACN, NH₄HCO₃ 0.01 mmol in water, 20% to 40% gradient in 20 min; detector, UV 254 nm. The fractions contained desired product were combined and concentrated to afford 1-[(2R,4S)-4-[4-amino-3-[2-(2-ethyl-4,6-difluoroindazol-5-yl)ethynyl]pyrazolo[3,4-d]pyrimidin-1-yl]-2-(methoxymethyl)pyrrolidin-1-yl]prop-2-en-1-one (10.2 mg, 6%) as a white solid ESI calculated for C₂₅H₂₄F₂N₈O₂ [M+H]⁺, 507.2; found 507.2. ¹H NMR (300 MHz, DMSO-d₆) δ 8.33 (d, J=1.2 Hz, 1H), 7.59-7.36 (m, 1H), 7.14-6.95 (m, 1H), 6.68-6.45 (m, 1H), 6.18-5.96 (m, 1H), 5.89-5.48 (m, 2H), 4.62 (q, J=7.2 Hz, 3H), 4.28-3.77 (m, 2H), 3.71-3.43 (m, 2H), 3.34 (s, 3H), 2.67-2.45 (m, 1H), 2.40-2.45 (m, 1H), 1.69-1.33 (m, 3H).

### Example 50: 1-((2R,4S)-4-(4-amino-3-((4,6-difluoro-1-methyl-1H-indazol-5-yl)ethynyl)-1H-pyrazolo[4,3-c]pyridin-1-yl)-2-(methoxymethyl)pyrrolidin-1-yl)prop-2-en-1-one

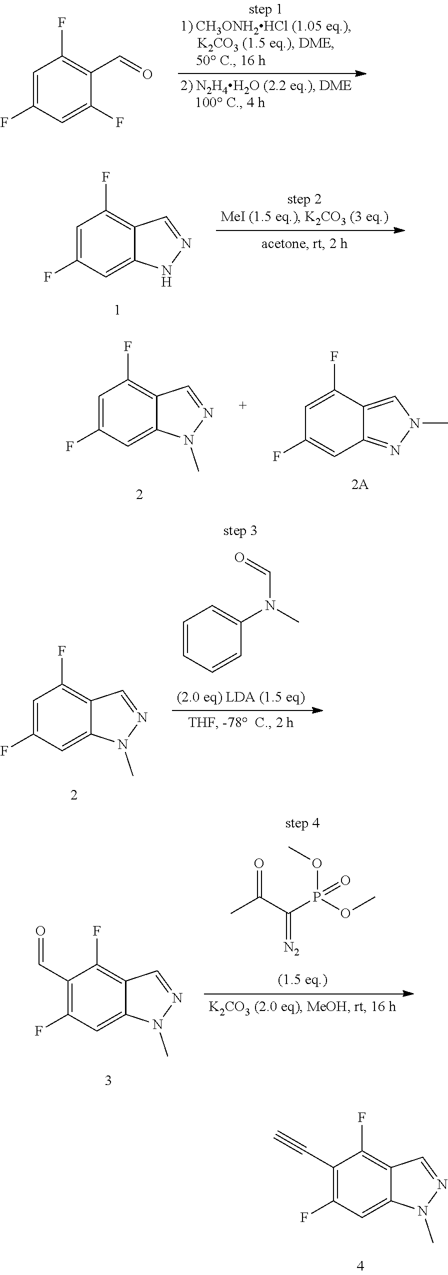

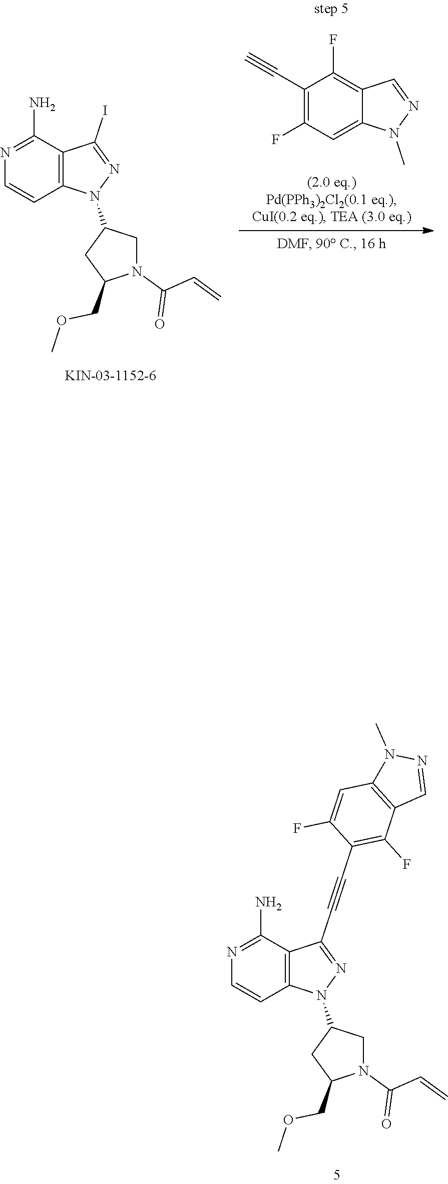

### Step 1: 4,6-difluoro-1H-indazole

To a mixture of 2,4,6-trifluorobenzaldehyde (20.00 g, 124.93 mmol) and O-methylhydroxylamine hydrochloride (10.96 g, 131.17 mmol) in DME (260.00 mL) was added K₂CO₃ (26.07 g, 188.64 mmol). The reaction mixture was stirred for overnight at 50° C. under argon atmosphere. The mixture was allowed to cool down to room temperature. The resulting mixture was filtered, the filter cake was washed with DCM (3×100 mL). The filtrate was concentrated under reduced pressure. To the above mixture was added NH₂NH₂·H₂O (6.68 mL, 133.42 mmol) and DME (260.00 mL) at room temperature. The resulting mixture was stirred for additional 2 h at 100° C. LCMS showed 40% of product. NH₂NH₂·H₂O (6.68 mL, 133.42 mmol) was added. The resulting mixture was stirred for additional 2 h at 100° C. LCMS showed major product. The resulting mixture was concentrated under reduced pressure. The resulting mixture was diluted with water (100 mL), extracted with EtOAc (3×300 mL). The combined organic layers were washed with water (2×200 mL), dried over anhydrous Na₂SO₄ and filtered. The filtrate was concentrated under reduced pressure. The residue was purified by silica gel column chromatography, eluted with CH₂Cl₂/PE (9:1). The fractions contained desired product were combined and concentrated to afford 4,6-difluoro-1H-indazole (15.4 g, 80%) as an off-white solid. MS ESI calculated for C₇H₄F₂N₂[M−H]⁻, 153.03, found 153.00.

### Step 2: 4,6-difluoro-2-methylindazole and 6-difluoro-2-methylindazole

To a stirred mixture of 4,6-difluoro-1H-indazole (0.50 g, 3.24 mmol) and K₂CO₃ (1.35 g, 9.73 mmol) in acetone (10.00 mL) was added CH₃I (0.30 mL, 2.12 mmol) dropwise at 0° C. under argon atmosphere. The resulting mixture was stirred for 2 h. The resulting mixture was filtered, the filter cake was washed with DCM (3×50 mL). The filtrate was concentrated under reduced pressure. The residue was purified by silica gel column chromatography, eluted with PE/EtOAc (5:1). The fractions contained desired product were combined and concentrated to afford 4,6-difluoro-1-methylindazole (0.29 g, 53%) as an off-white solid. H NMR (400 MHz, Chloroform-d) δ 8.02 (d, J=0.8 Hz, 1H), 6.88-6.86 (m, 1H), 6.65-6.62 (m, 1H), 4.04 (s, 3H). The fractions contained desired product were combined and concentrated to afford 4,6-difluoro-2-methylindazole (0.20 g, 37%) as an off-white solid. H NMR (400 MHz, Chloroform-d) δ 7.95 (s, 1H), 7.15-7.10 (m, 1H), 6.59-6.55 (m, 1H), 4.20 (s, 3H).

### Step 3: 4,6-difluoro-1-methylindazole-5-carbaldehyde

To a stirred solution of LDA (10.70 mL, 21.40 mmol) in THF (100.00 mL) was added 4,6-difluoro-1-methylindazole (2.4 g, 14.27 mmol) in THF (25.00 mL) dropwise at −78° C. under argon atmosphere. The reaction mixture was stirred for 2 h at −78° C. To the above mixture was added N-methyl-N-phenylformamide (3.61 mL, 28.85 mmol) dropwise. The resulting mixture was stirred for additional 2 h at −78° C. The resulting mixture was quenched with sat. NH₄Cl (aq.) at 0° C., extracted with EtOAc (2×300 mL). The combined organic layers were washed with brine (2×200 mL), dried over anhydrous Na₂SO₄ and filtered. The filtrate was concentrated under reduced pressure. The residue was purified by silica gel column chromatography, eluted with PE/EtOAc (4:1). The fractions contained desired product were combined and concentrated to afford 4,6-difluoro-1-methylindazole-5-carbaldehyde (2 g, 71%) as a light yellow solid. H NMR (400 MHz, Chloroform-d) δ 10.40 (t, J=1.2 Hz, 1H), 8.19 (d, J=1.2 Hz, 1H), 6.95 (dd, J=10.8, 1.2 Hz, 1H), 4.08 (s, 3H).

### Step 4: 5-ethynyl-4,6-difluoro-1-methylindazole

To a stirred mixture of 4,6-difluoro-1-methylindazole-5-carbaldehyde (2.00 g, 10.20 mmol) and K₂CO₃ (2.82 g, 20.39 mmol) in MeOH (60.00 mL) was added dimethyl (1-diazo-2-oxopropyl)phosphonate (2.30 mL, 11.95 mmol) dropwise at room temperature under argon atmosphere. The reaction mixture was stirred for 16 h. The resulting mixture was diluted with water (150 mL), extracted with EtOAc (2×300 mL). The combined organic layers were washed with brine (2×200 mL), dried over anhydrous Na₂SO₄ and filtered. The filtrate was concentrated under reduced pressure. The residue was purified by silica gel column chromatography, eluted with PE/EtOAc (5:1). The fractions contained desired product were combined and concentrated to afford 5-ethynyl-4,6-difluoro-1-methylindazole (0.84 g, 38%) as a light brown solid. H NMR (400 MHz, Chloroform-d) δ 8.08-8.03 (m, 1H), 6.92 (d, J=8.4 Hz, 1H), 4.04 (s, 3H), 3.50 (s, 1H).

### Step 5: 1-[(2R,4S)-4-[4-amino-3-[2-(4,6-difluoro-1-methylindazol-5-yl)ethynyl]pyrazolo[4,3-c]pyridin-1-yl]-2-(methoxymethyl)pyrrolidin-1-yl]prop-2-en-1-one

To a mixture of 1-[(2R,4S)-4-[4-amino-3-iodopyrazolo[4,3-c]pyridin-1-yl]-2-(methoxymethyl)pyrrolidin-1-yl]prop-2-en-1-one (0.15 g, 0.35 mmol), 5-ethynyl-4,6-difluoro-1-methylindazole (0.13 g, 0.70 mmol), CuI (13.37 mg, 0.07 mmol) and Pd(PPh₃)₂Cl₂ (24.64 mg, 0.04 mmol) in DMF (2.00 mL) was added TEA (0.15 mL, 1.08 mmol). The reaction mixture was degassed with argon for three times and stirred for 16 h at 90° C. The resulting mixture was diluted with water (50 mL), extracted with EtOAc (3×50 mL). The combined organic layers were washed with water (2×30 mL), dried over anhydrous Na₂SO₄ and filtered. The filtrate was concentrated under reduced pressure. The residue was purified by reverse phase flash with the following conditions Column: SunFire Prep C18 OBD Column, 19×150 mm 5 um 10 nm; Mobile Phase A: Water (0.1% FA), Mobile Phase B: ACN; Flow rate: 20 mL/min; Gradient: 30 B to 50 B in 6 min; 210/254 nm; RT1: 5.56. The fractions contained desired product were combined and concentrated to afford 1-[(2R,4S)-4-[4-amino-3-[2-(4,6-difluoro-1-methylindazol-5-yl)ethynyl]pyrazolo[4,3-c]pyridin-1-yl]-2-(methoxymethyl)pyrrolidin-1-yl]prop-2-en-1-one (57 mg, 33%) as an off-white solid. MS ESI calculated for C₂₅H₂₃F₂N₇O₂ [M+H]⁺, 492.19, found 492.20. H NMR (400 MHz, DMSO-d₆) δ 8.35 (d, J=0.8 Hz, 1H), 7.82 (dd, J=6.0, 1.2 Hz, 1H), 7.74 (d, J=9.6 Hz, 1H), 6.98 (d, J=6.0 Hz, 1H), 6.82-6.53 (m, 1H), 6.51 (s, 2H), 6.19-6.15 (m, 1H), 5.69-5.65 (m, 1H), 5.50 (q, J=6.4 Hz, 1H), 4.67-4.39 (m, 1H), 4.07 (s, 3H), 3.95-3.86 (m, 1H), 3.84-3.77 (m, 1H), 3.64-3.57 (m, 1H), 3.56-3.47 (m, 1H), 3.33 (d, J=4.8 Hz, 3H), 2.74-2.55 (m, 1H), 2.45-2.31 (m, 1H).

### Example 51: 1-((2R,4S)-4-(4-amino-3-((2-ethyl-4,6-difluoro-2H-indazol-5-yl)ethynyl)-1H-pyrazolo[4,3-c]pyridin-1-yl)-2-(methoxymethyl)pyrrolidin-1-yl)prop-2-en-1-one

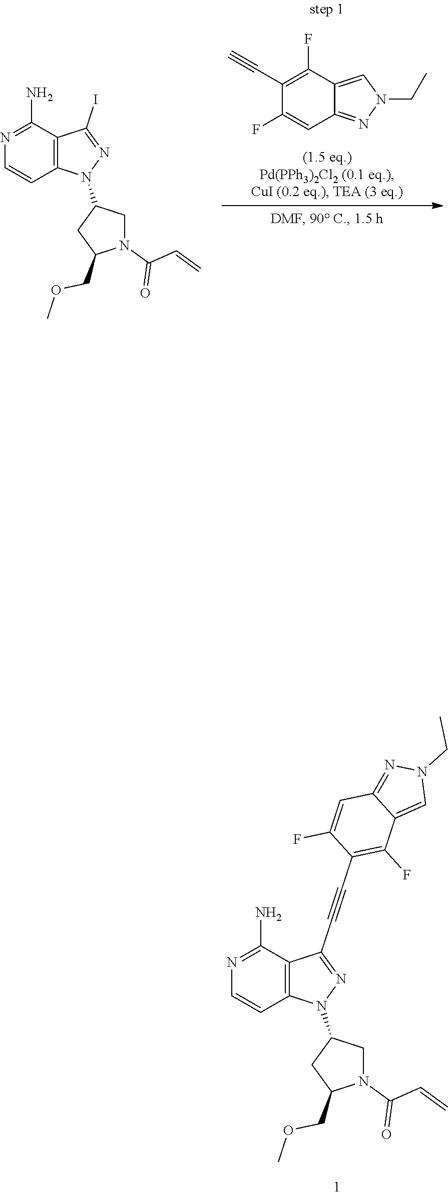

To a stirred solution of 1-[(2R)-4-[4-amino-3-iodopyrazolo[4,3-c]pyridin-1-yl]-2-(methoxymethyl)pyrrolidin-1-yl]prop-2-en-1-one (0.1 g, 0.23 mmol), 2-ethyl-5-ethynyl-4,6-difluoroindazole (72.39 mg, 0.35 mmol), Pd(PPh3)₂Cl₂ (16.43 mg, 0.02 mmol) and CuI (8.92 mg, 0.05 mmol) in DMF (2.00 mL) was added TEA (71.05 mg, 0.70 mmol). The reaction mixture was degassed with argon for three times and stirred for 1.5 h at 90° C. The residue was purified by reverse flash chromatography with the following conditions: column, C18 silica gel; mobile phase ACN, NH₄HCO₃ 0.01 mmol in water, 20% to 40% gradient in 20 min; detector, UV 254 nm. The fractions contained desired product were combined and concentrated to afford 1-[(2R,4S)-4-[4-amino-3-[2-(2-ethyl-4,6-difluoroindazol-5-yl)ethynyl]pyrazolo[4,3-c]pyridin-1-yl]-2-(methoxymethyl)pyrrolidin-1-yl]prop-2-en-1-one (11.3 mg, 9%) as a light yellow solid. ESI calculated for C₂₆H₂₅F₂N₇O₂ [M+H]⁺, 506.2; found 506.2. ¹H NMR (400 MHz, DMSO-d₆) δ 7.85 (d, J=7.2 Hz, 1H), 7.46-7.25 (m, 2H), 7.14-6.95 (m, 1H), 6.65-6.43 (m, 1H), 6.29-6.14 (m, 1H), 5.80-5.48 (m, 2H), 4.61 (q, J=7.3 Hz, 3H), 4.18-3.78 (m, 2H), 3.68-3.48 (m, 2H), 3.32 (d, J=6.2 Hz, 3H), 2.81-2.57 (m, 1H), 2.46-2.29 (m, 1H), 1.55 (t, J=7.2 Hz, 3H).

### Example 52: 2-((2R,4S)-1-acryloyl-4-(4-amino-3-((1-ethyl-2-methyl-1H-benzo[d]imidazol-5-yl)ethynyl)-1H-pyrazolo[3,4-d]pyrimidin-1-yl)pyrrolidin-2-yl)acetonitrile

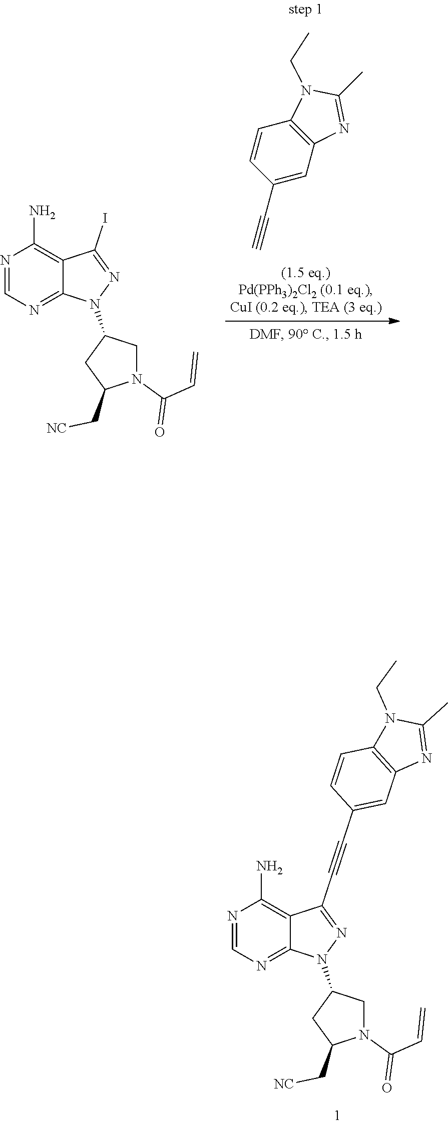 [SMILES: C=CC(=O)N1C[C@@H](n2nc(I)c3c(N)ncnc32)C[C@@H]1CC#N ; C#Cc1ccc2c(c1)nc(C)n2CC ; C=CC(=O)N1C[C@@H](n2nc(C#Cc3ccc4c(c3)nc(C)n4CC)c3c(N)ncnc32)C[C@@H]1CC#N]

To a stirred solution of 2-[(2R,4S)-4-[4-amino-3-iodopyrazolo[3,4-d]pyrimidin-1-yl]-1-(prop-2-enoyl)pyrrolidin-2-yl]acetonitrile (0.15 g, 0.35 mmol), 1-ethyl-5-ethynyl-2-methyl-1,3-benzodiazole (97.95 mg, 0.53 mmol), Pd(PPh3)₂Cl₂ (24.88 mg, 0.04 mmol) and CuI (13.50 mg, 0.07 mmol) in DMF (2.00 mL) was added TEA (0.11 g, 1.06 mmol). The reaction mixture was degassed with argon for three times and stirred for 1.5 h at 90° C. The resulting mixture was purified by reverse flash chromatography with the following conditions: column, C18 silica gel; mobile phase ACN, NH₄HCO₃ 0.01 mmol in water, 20% to 40% gradient in 20 min; detector, UV 254 nm. The fractions contained desired product were combined and concentrated to afford 2-[(2R,4S)-4-[4-amino-3-[2-(1-ethyl-2-methyl-1,3-benzodiazol-5-yl)ethynyl]pyrazolo[3,4-d]pyrimidin-1-yl]-1-(prop-2-enoyl)pyrrolidin-2-yl]acetonitrile (29.5 mg, 17%) as a white solid. ESI calculated for C₂₆H₂₅N₉O [M+H]⁺, 480.2; found 480.2. ¹H NMR (400 MHz, DMSO-d₆) δ 8.29 (s, 1H), 7.95 (s, 2H), 7.71-7.42 (m, 2H), 6.71-6.52 (m, 2H), 6.32-6.14 (m, 1H), 5.90-5.64 (m, 2H), 4.96-4.46 (m, 1H), 4.26 (q, J=7.2 Hz, 2H), 4.21-3.78 (m, 2H), 3.22-3.18 (m, 1H), 3.10-2.94 (m, 1H), 2.93-2.65 (m, 1H), 2.57 (s, 3H), 2.40-2.01 (m, 1H), 1.31 (t, J=7.2 Hz, 3H).

### Example 53: 2-[(2R,4S)-4-[4-Amino-3-[2-(1-ethyl-2-methyl-1,3-benzodiazol-5-yl)ethynyl]pyrazolo[4,3-c]pyridin-1-yl]-1-(prop-2-enoyl)pyrrolidin-2-yl]acetonitrile; formic acid

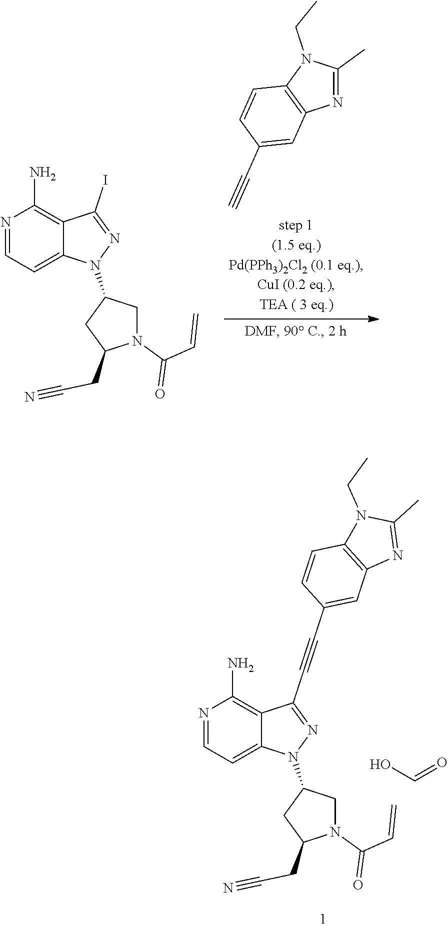

To a stirred mixture of 2-[(2R,4S)-4-[4-amino-3-iodopyrazolo[4,3-c]pyridin-1-yl]-1-(prop-2-enoyl)pyrrolidin-2-yl]acetonitrile (70.00 mg, 0.17 mmol), 1-ethyl-5-ethynyl-2-methyl-1,3-benzodiazole (61.09 mg, 0.33 mmol), Pd(PPh₃)₂Cl₂ (11.64 mg, 0.02 mmol) and CuI (6.31 mg, 0.03 mmol) in DMF (1.50 mL) was added TEA (50.33 mg, 0.50 mmol). The reaction mixture was degassed with argon for three times and stirred for 2 h at 90° C. The resulting mixture was purified by reverse flash chromatography with the following conditions: column: C18 silica gel; mobile phase: ACN in water (10 mmol/L NH₄HCO₃), 25% to 40% gradient in 20 min; detector: UV 254 nm. The fractions contained desired product were combined and concentrated to afford 2-[(2R,4S)-4-[4-amino-3-[2-(1-ethyl-2-methyl-1,3-benzodiazol-5-yl)ethynyl]pyrazolo[4,3-c]pyridin-1-yl]-1-(prop-2-enoyl)pyrrolidin-2-yl]acetonitrile; formic acid (18.50 mg, 21%) as a white solid. MS ESI calculated for C₂₇H₂₆N₈O [M+H]⁺, 479.20, found 479.21; ¹H NMR (400 MHz, DMSO-d₆) δ 8.17 (s, 1H), 7.82 (d, J=6.1 Hz, 1H), 7.62 (d, J=8.4 Hz, 1H), 7.48 (dd, J=8.3, 1.5 Hz, 1H), 6.97 (d, J=6.1 Hz, 1H), 6.60 (dd, J=16.7, 10.3 Hz, 1H), 6.41 (s, 2H), 6.21-5.92 (m, 1H), 5.75-5.63 (m, 1H), 5.59-5.23 (m, 1H), 4.55-4.32 (m, 1H), 4.26 (q, J=7.2 Hz, 2H), 4.15-4.10 (m, 1H), 3.96-3.75 (m, 1H), 3.19-3.13 (m, 1H), 3.06-3.02 (m, 1H), 2.80-2.66 (m, 1H), 2.58 (s, 3H), 2.40-2.35 (m, 1H), 1.31-1.19 (m, 3H).

### Example 54: 1-[(2R,4S)-4-[4-Amino-3-[2-(4,6-difluoro-1-methyl-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one

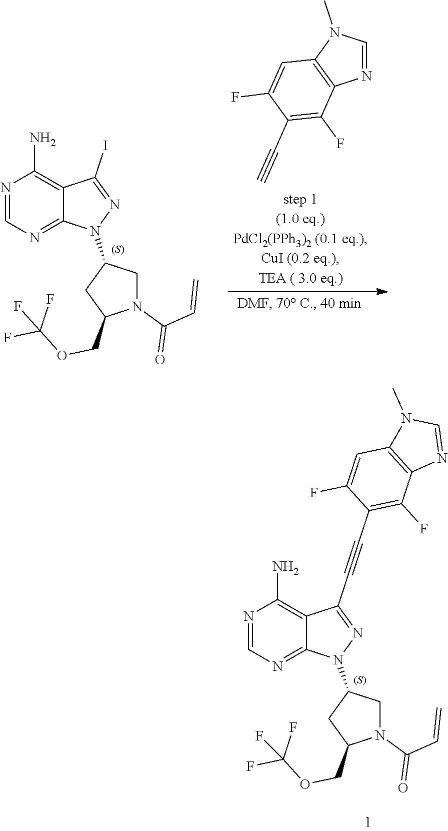

To a stirred mixture of 1-[(2R,4S)-4-[4-amino-3-iodopyrazolo[3,4-d]pyrimidin-1-yl]-2-[(trifluoromethoxy)methyl]pyrrolidin-1-yl]prop-2-en-1-one (0.13 g, 0.27 mmol) and 5-ethynyl-4,6-difluoro-1-methyl-1,3-benzodiazole (51.81 mg, 0.27 mmol) in DMF (2.50 mL) were added Pd(PPh₃)₂Cl₂ (18.92 mg, 0.02 mmol), CuI (10.27 mg, 0.05 mmol) and TEA (81.84 mg, 0.80 mmol) at room temperature. The reaction mixture was degassed with argon for three times and stirred for 40 min at 70° C. The resulting mixture was concentrated under reduced pressure. The residue was purified by silica gel column chromatography, eluted with in DCM/MeOH (10/1) to afford crude product. The crude product was purified by Prep-HPLC with the following conditions: Column: Atlantis Prep T3 OBD Column, 19×250 mm 10 μm; Mobile Phase A: water (0.1% FA), Mobile Phase B: ACN; Flow rate: 20 mL/min; Gradient: 25 B to 50 B in 6 min; 210/254 nm; RT1:5.56 min. The fractions contained desired product were combined and concentrated to afford 1-[(2R,4S)-4-[4-amino-3-[2-(4,6-difluoro-1-methyl-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one (75.70 mg, 50%) as an off-white solid. MS ESI calculated for C₂₄H₁₉F₅N₈O₂ [M+H]⁺, 547.16, found 547.00; ¹H NMR (400 MHz, DMSO-d₆) δ 8.38 (s, 1H), 8.32 (s, 1H), 7.66 (d, J=9.1 Hz, 1H), 6.68-6.62 (m, 1H), 6.20-6.16 (m, 1H), 5.86-5.50 (m, 2H), 4.73-4.61 (m, 1H), 4.46-4.44 (m, 1H), 4.39-4.20 (m, 1H), 4.09-4.05 (m, 2H), 3.87 (s, 3H), 2.90-2.65 (m, 1H), 2.47-2.45 (m, 1H).

### Example 55: 1-[(2R,4S)-4-[4-Amino-3-[2-(4,6-difluoro-1-methyl-1,3-benzodiazol-5-yl) ethynyl]pyrazolo[3,4-d] pyrimidin-1-yl]-2-(methoxymethyl) pyrrolidin-1-yl] prop-2-en-1-one

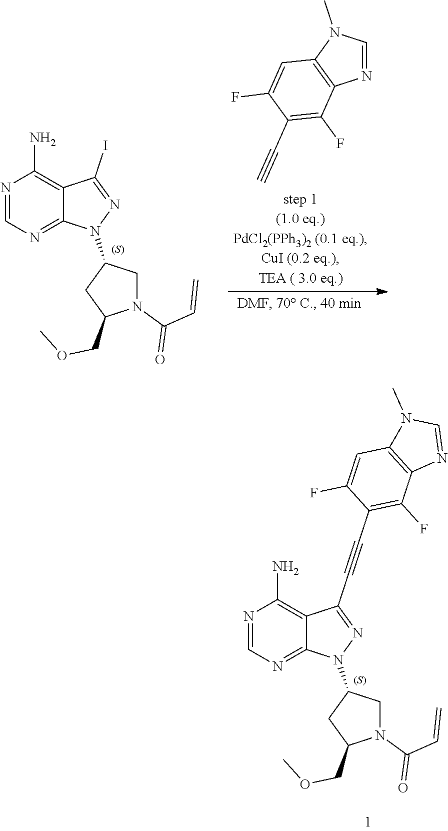

To a stirred mixture of 5-ethynyl-4,6-difluoro-1-methyl-1,3-benzodiazole (58.34 mg, 0.30 mmol), 1-[(2R,4S)-4-[4-amino-3-iodopyrazolo[3,4-d]pyrimidin-1-yl]-2-(methoxymethyl) pyrrolidin-1-yl] prop-2-en-1-one (0.13 g, 0.30 mmol), CuI (11.56 mg, 0.06 mmol) and Pd(PPh₃)₂Cl₂ (21.31 mg, 0.03 mmol) in DMF (2.00 mL) was added TEA (92.16 mg, 0.91 mmol). The reaction mixture was degassed with argon for three times and stirred for 40 min at 70° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by reverse phase chromatography with the following conditions: Column: Spherical C18, 20-40 um, 40 g; Mobile Phase A: water (10 mmol/L NH₄HCO₃), Mobile Phase B: ACN; Flow rate: 40 mL/min; Gradient (B %): 5%-40% within 35 min, Detector: UV 254/220 nm. The fractions contained desired product were combined and concentrated to afford 1-[(2R,4S)-4-[4-amino-3-[2-(4,6-difluoro-1-methyl-1,3-benzodiazol-5-yl) ethynyl] pyrazolo[3,4-d] pyrimidin-1-yl]-2-(methoxymethyl) pyrrolidin-1-yl] prop-2-en-1-one (54.40 mg, 36%) as an off-white solid. MS ESI calculated for C₂₄H₂₂F₂N₈O₂ [M+H]⁺, 493.18, found 493.20; ¹H NMR (400 MHz, DMSO-d₆) δ 8.39 (s, 1H), 8.31 (d, J=2.3 Hz, 1H), 7.66 (d, J=9.1 Hz, 1H), 6.79-6.54 (m, 1H), 6.20-6.14 (m, 1H), 5.75-5.57 (m, 2H), 4.54 (d, J=54.8 Hz, 1H), 4.14-3.91 (m, 2H), 3.87 (s, 3H), 3.69-3.46 (m, 2H), 3.32-3.31 (m, 3H), 2.73-2.69 (m, 1H), 2.43-2.38 (m, 1H).

### Example 56: 1-((2R,4S)-4-(4-Amino-3-((1-methyl-1H-benzo[d]imidazol-5-yl)ethynyl)-1H-pyrazolo[3,4-d]pyrimidin-1-yl)-2-((trifluoromethoxy)methyl)pyrrolidin-1-yl)prop-2-en-1-one

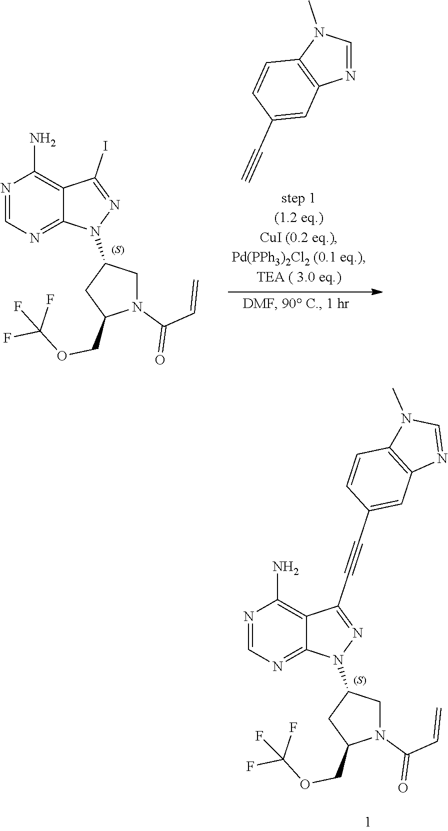

To a stirred mixture of 1-((2R,4S)-4-(4-amino-3-ethynyl-1H-pyrazolo[3,4-d]pyrimidin-1-yl)-2-((trifluoromethoxy)methyl)pyrrolidin-1-yl)prop-2-en-1-one (0.20 g, 0.42 mmol) and 5-iodo-1-methyl-1H-benzo[d]imidazole (77.00 mg, 0.49 mmol) in DMF (3.00 mL) were added Pd(PPh₃)₂Cl₂ (28.76 mg, 0.04 mmol), CuI (16.37 mg, 0.08 mmol) and TEA (0.17 mL, 1.30 mmol) at room temperature. The reaction mixture was degassed with nitrogen for three times and stirred for 1 h at 90° C. The resulting mixture was concentrated under reduced pressure. The residue was purified by Prep-HPLC with the following conditions: Column: Atlantis Prep T3 OBD Column, 19×250 mm 10 μm; Mobile Phase A: water (0.1% FA), Mobile Phase B: ACN; Flow rate: 20 mL/min; Gradient: 15 B to 55 B in 6 min; 210/254 nm; RT: 5.56 min. The fractions contained desired product were combined and concentrated to afford 1-[(2R,4S)-4-[4-amino-3-[2-(1-methyl-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one (66.00 mg, 27%) as an off-white solid. MS ESI calculated for C₂₄H₂₁F₃N₈O₂ [M+H]⁺, 511.17, found 511.25; ¹H NMR (400 MHz, DMSO-d₆) δ 8.56 (s, 1H), 8.30 (d, J=2.6 Hz, 1H), 8.14 (s, 1H), 7.75 (d, J=8.4 Hz, 2H), 6.59 (d, J=16.6 Hz, 1H), 6.19 (d, J=16.7 Hz, 1H), 5.71 (d, J=10.3 Hz, 1H), 5.60-5.69 (m, 1H), 4.62 (d, J=7.4 Hz, 1H), 4.48-4.45 (m, 1H), 4.36-4.23 (m, 1H), 4.13-4.09 (m, 1H), 4.05-4.02 (m, 1H), 3.92 (s, 3H), 2.65-2.60 (m, 1H), 2.43-2.39 (m, 1H).

### Example 57: 1-[(2R,4S)-4-[4-Amino-3-[2-(6-fluoro-1-methyl-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one

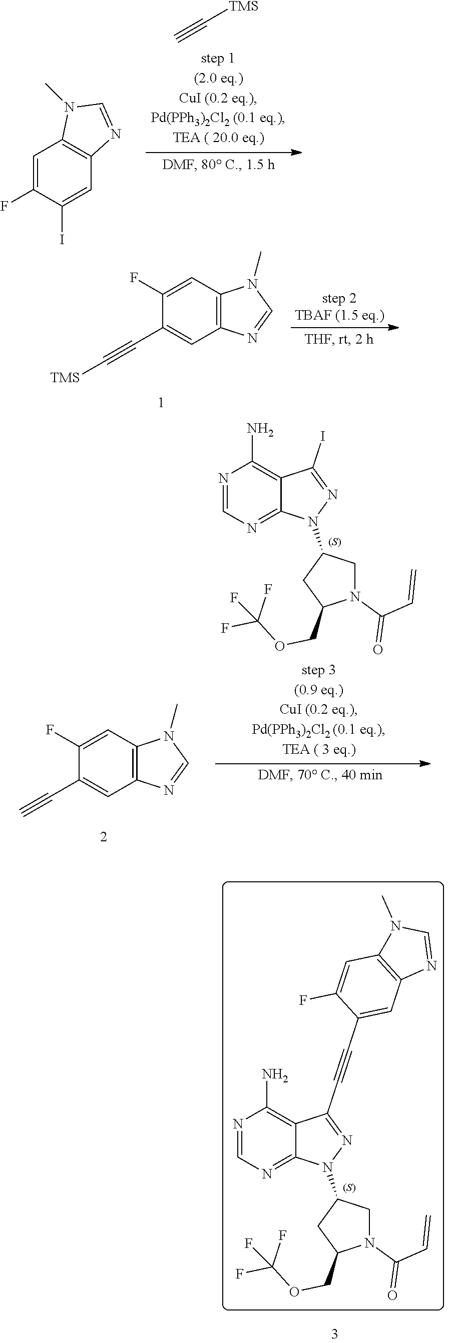

1-[(2R,4S)-4-[4-amino-3-[2-(6-fluoro-1-methyl-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one. MS ESI calculated for C₂₄H₂₀F₄N₈O₂ [M+H]⁺, 529.16, found 529.15; ¹H NMR (400 MHz, DMSO-d₆) δ 8.32 (d, J=11.3 Hz, 2H), 8.15 (d, J=6.3 Hz, 1H), 7.71 (d, J=9.8 Hz, 1H), 6.59 (dd, J=16.8, 10.3 Hz, 1H), 6.19 (dd, J=16.7, 2.4 Hz, 1H), 5.79-5.66 (m, 1H), 5.66-5.58 (m, 1H), 4.61 (s, 1H), 4.51-4.46 (m, 1H), 4.36-4.24 (m, 1H), 4.15-3.99 (m, 2H), 3.85 (s, 3H), 2.72-2.65 (m, 1H), 2.46-2.43 (m, 1H).

### Example 58: 1-((2R,4S)-4-(4-Amino-3-((1-methyl-1H-benzo[d]imidazol-5-yl)ethynyl)-1H-pyrazolo[4,3-c]pyridin-1-yl)-2-((trifluoromethoxy)methyl)pyrrolidin-1-yl)prop-2-en-1-one formate

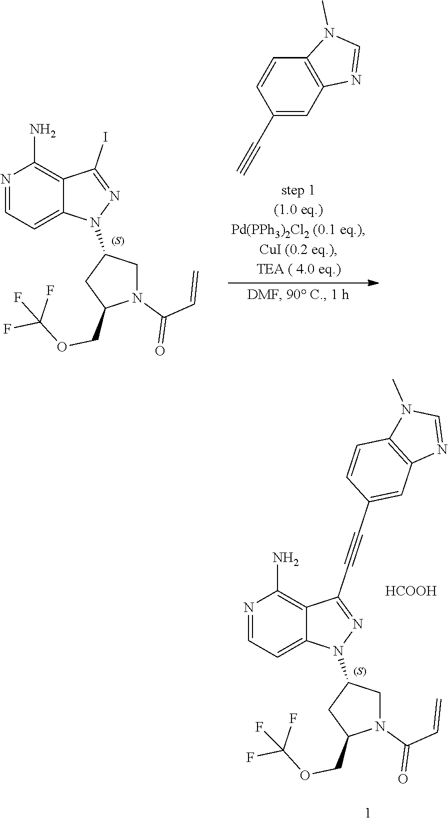

To a stirred mixture of 1-[(2R,4S)-4-[4-amino-3-iodopyrazolo[4,3-c]pyridin-1-yl]-2-[(trifluoromethoxy)methyl]pyrrolidin-1-yl]prop-2-en-1-one (0.23 g, 0.48 mmol), 5-ethynyl-1-methyl-1,3-benzodiazole (74.65 mg, 0.48 mmol), CuI (18.21 mg, 0.10 mmol) and Pd(PPh₃)₂Cl₂ (33.55 mg, 0.05 mmol) in DMF (2.50 mL) was added TEA (0.20 mL, 1.97 mmol). The reaction mixture was degassed with argon for three times and stirred for 1 h at 90° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by Prep-HPLC with the following conditions: Column: SunFire Prep C18 OBD Column, 19×150 mm 5 μm; Mobile Phase A: water (0.1% FA), Mobile Phase B: MeOH-HPLC; Flow rate: 20 mL/min; Gradient: 25 B to 30 B in 6 min; 210/254 nm; RT1: 5.56 min. The fractions contained desired product were combined and concentrated to afford 1-((2R,4S)-4-(4-amino-3-((1-methyl-1H-benzo[d]imidazol-5-yl)ethynyl)-1H-pyrazolo[4,3-c]pyridin-1-yl)-2-((trifluoromethoxy)methyl)pyrrolidin-1-yl)prop-2-en-1-one formate (63.30 mg, 24%) as an off-white solid. MS ESI calculated for C₂₆H₂₄F₃N₇O₄ [M+H−46]⁺, 510.18, found 510.20; ¹H NMR (400 MHz, DMSO-d₆) δ 8.33 (s, 1H), 8.14-8.02 (m, 2H), 7.69-7.66 (m, 2H), 7.57-7.55 (m, 1H), 6.97 (d, J=5.9 Hz, 1H), 6.62-6.55 (m, 1H), 6.43 (s, 2H), 6.21 (dd, J=16.9, 2.3 Hz, 1H), 5.79-5.67 (m, 1H), 5.55-5.49 (m, 1H), 4.97-4.51 (m, 1H), 4.51-4.19 (m, 2H), 4.10-3.94 (m, 2H), 3.88 (s, 3H), 2.71-2.59 (m, 1H), 2.46-2.39 (m 1H).

### Example 59: 1-((2R,4S)-4-(4-amino-3-((6-fluoro-1-methyl-1H-benzo[d]imidazol-5-yl)ethynyl)-1H-pyrazolo[4,3-c]pyridin-1-yl)-2-((trifluoromethoxy)methyl)pyrrolidin-1-yl)prop-2-en-1-one formate

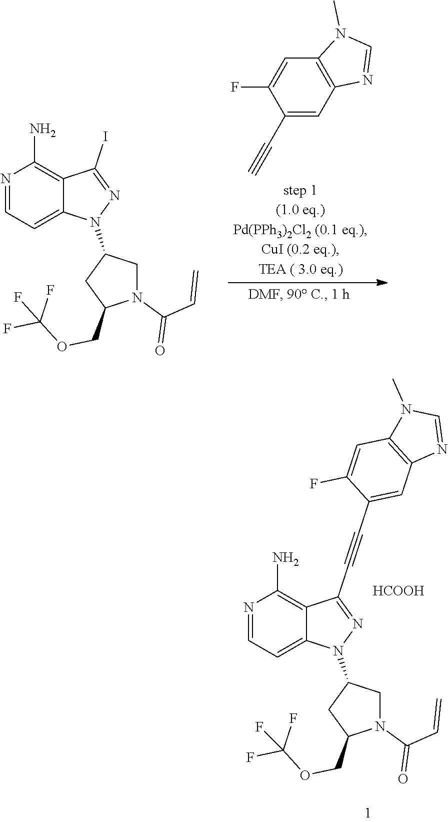 [SMILES: C=CC(=O)N1C[C@@H](n2nc(I)c3c(N)nccc32)C[C@@H]1COC(F)(F)F ; C#Cc1cc2ncn(C)c2cc1F ; C=CC(=O)N1C[C@@H](n2nc(C#Cc3cc4ncn(C)c4cc3F)c3c(N)nccc32)C[C@@H]1COC(F)(F)F ; "HCOOH"]

To a stirred mixture of 1-[(2R,4S)-4-[4-amino-3-iodopyrazolo[4,3-c]pyridin-1-yl]-2-[(trifluoromethoxy)methyl]pyrrolidin-1-yl]prop-2-en-1-one (0.20 g, 0.42 mmol), 5-ethynyl-6-fluoro-1-methyl-1,3-benzodiazole (72.39 mg, 0.42 mmol), CuI (15.83 mg, 0.08 mmol) and Pd(PPh₃)₂Cl₂ (29.17 mg, 0.04 mmol) in DMF (2.00 mL) was added TEA (0.17 mL, 1.22 mmol). The reaction mixture was degassed with argon for three times and stirred for 1 h at 90° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by Prep-HPLC with the following conditions: Column: SunFire Prep C18 OBD Column, 19×150 mm 5 m; Mobile Phase A: water (0.1% FA), Mobile Phase B: MeOH-HPLC; Flow rate: 20 mL/min; Gradient: 25 B to 30 B in 6 min; 210/254 nm; RT1: 5.56 min. The fractions contained desired product were combined and concentrated to afford 1-((2R,4S)-4-(4-amino-3-((6-fluoro-1-methyl-1H-benzo[d]imidazol-5-yl)ethynyl)-1H-pyrazolo[4,3-c]pyridin-1-yl)-2-((trifluoromethoxy)methyl)pyrrolidin-1-yl)prop-2-en-1-one formate (39.20 mg, 16%) as an off-white solid. MS ESI calculated for C₂₆22 3F4N₇O₄ [M+H−46]⁺, 528.17, found 528.15; ¹H NMR (400 MHz, DMSO-d₆) δ 8.34 (s, 1H), 8.14 (s, 1H), 8.12-8.02 (m, 1H), 7.82-7.77 (m, 1H), 7.72-7.65 (m, 1H), 7.03 (dd, J=6.4, 4.0 Hz, 1H), 6.73 (s, 2H), 6.62-6.55 (m, 1H), 6.21-6.16 (m, 1H), 5.79-5.67 (m, 1H), 5.56-5.48 (m, 1H), 4.86-4.61 (m, 1H), 4.51-4.20 (m, 2H), 4.10-4.08 (m, 1H), 3.98-3.95 (m, 1H), 3.85 (s, 3H), 2.82-2.60 (m, 1H), 2.48-2.40 (m, 1H).

### Example 60: 1-[(2R,4S)-4-[4-Amino-3-[2-(4,6-difluoro-1-methyl-1,3-benzodiazol-5-yl)ethynyl]pyrazolo[4,3-c]pyridin-1-yl]-2-[(trifluoromethoxy)methyl]pyrrolidin-1-yl]prop-2-en-1-one

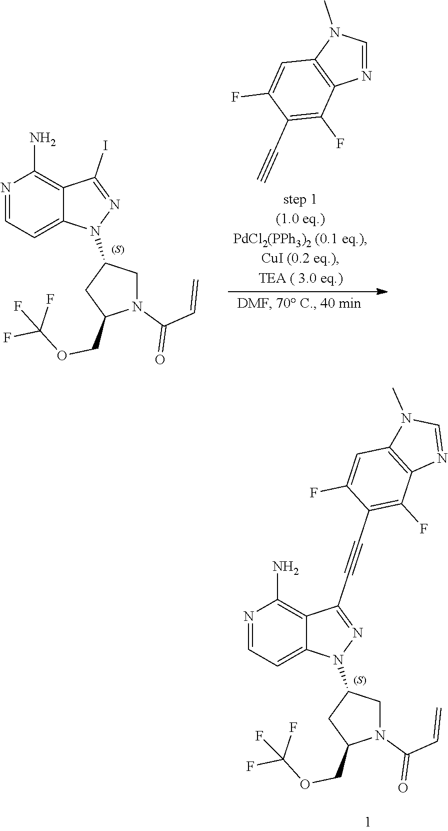

To a stirred mixture of 1-[(2R,4S)-4-[4-amino-3-iodopyrazolo[4,3-c]pyridin-1-yl]-2-[(trifluoromethoxy)methyl]pyrrolidin-1-yl]prop-2-en-1-one (0.13 g, 0.27 mmol) and 5-ethynyl-4,6-difluoro-1-methyl-1,3-benzodiazole (51.91 mg, 0.27 mmol) in DMF (2.50 mL) were added Pd(PPh₃)₂Cl₂ (18.96 mg, 0.02 mmol), CuI (10.29 mg, 0.05 mmol) and TEA (82.01 mg, 0.81 mmol) at room temperature. The reaction mixture was degassed with argon for three times and stirred for 40 min at 70° C. The resulting mixture was concentrated under reduced pressure. The residue was purified by silica gel column chromatography, eluted with DCM/MeOH (10/1) to afford crude product. The crude product was purified by Prep-HPLC with the following conditions Column: SunFire Prep C18 OBD Column, 19×150 mm 5 μm 10 nm; Mobile Phase A: water (0.1% FA), Mobile Phase B: MeOH-HPLC; Flow rate: 20 mL/min; Gradient: 25 B to 50 B in 6 min; 210/254 nm; RT1:5.53 min. The fractions contained desired product were combined and concentrated to afford 1-[(2R,4S)-4-[4-amino-3-[2-(4,6-difluoro-1-methyl-1,3-benzodiazol-5-yl)ethynyl]pyrazolo[4,3-c]pyridin-1-yl]-2-[(trifluoromethoxy)methyl]pyrrolidin-1-yl]prop-2-en-1-one (62.10 mg, 41%) as an off-white solid. MS ESI calculated for C₂₅H₂₀F₅N₇O₂ [M+H]⁺, 546.17, found 545.95; ¹H NMR (300 MHz, DMSO-d₆) δ 8.39 (s, 1H), 7.84 (d, J=6.2 Hz, 1H), 7.68 (d, J=9.2 Hz, 1H), 7.00 (d, J=6.2 Hz, 1H), 6.85-6.51 (m, 3H), 6.19 (dd, J=16.7, 2.3 Hz, 1H), 5.85-5.65 (m, 1H), 5.63-5.46 (m, 1H), 4.75-4.73 (m, 1H), 4.44-4.43 (m, 1H), 4.37-4.22 (m, 1H), 4.09-4.08 (m, 1H), 3.98-3.95 (m, 1H), 3.88 (s, 3H), 2.85-2.62 (m, 1H), 2.43-2.41 (m, 1H).

### Example 61: 1-[(2R,4S)-4-[4-Amino-5-[2-(4,6-difluoro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one

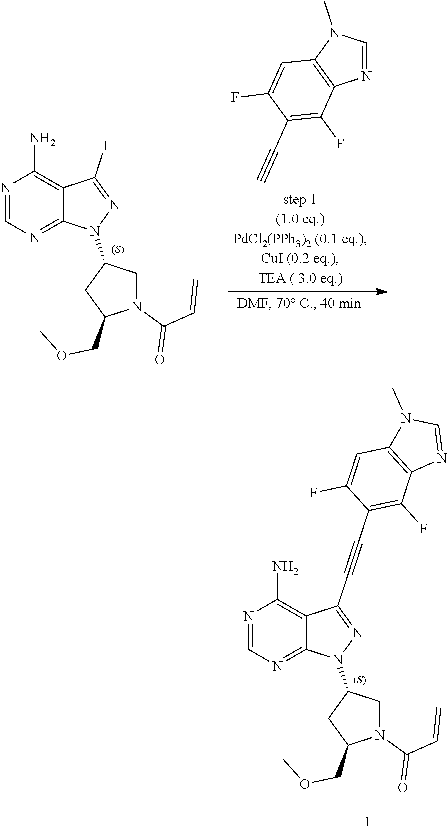

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (0.13 g, 0.30 mmol) and 5-ethynyl-4,6-difluoro-1-methyl-1,3-benzodiazole (58.47 mg, 0.30 mmol) in DMF (2.50 mL) were added Pd(PPh₃)₂Cl₂ (21.36 mg, 0.03 mmol), CuI (11.59 mg, 0.06 mmol) and TEA (92.37 mg, 0.91 mmol) at room temperature. The reaction mixture was degassed with argon for three times and stirred for 40 min at 70° C. The resulting mixture was concentrated under reduced pressure. The residue was purified by silica gel column chromatography, eluted with DCM/MeOH (10/1) to afford crude product. The crude product was purified by reverse flash chromatography with the following conditions: column: C18 silica gel; mobile phase: ACN in in water (10 mmol/L NH₄HCO₃), 10% to 50% gradient in 10 min; detector: UV 254 nm. The fractions contained desired product were combined and concentrated to afford 1-[(2R,4S)-4-[4-amino-5-[2-(4,6-difluoro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one (58.90 mg, 38%) as an off-white solid. MS ESI calculated for C₂₅H₂₃F₂N₇O₂ [M+H]⁺, 492.20, found 492.05; ¹H NMR (300 MHz, DMSO-d₆) δ 8.30 (s, 1H), 8.18 (d, J=2.4 Hz, 1H), 7.83 (d, J=12.6 Hz, 1H), 7.56 (d, J=9.1 Hz, 1H), 6.53-6.49 (m, 1H), 6.27-6.08 (m, 1H), 5.81-5.64 (m, 1H), 5.47-5.43 (m, 1H), 4.53-4.51 (m, 1H), 4.08-4.07 (m, 1H), 3.84-3.82 (m, 4H), 3.62-3.43 (m, 2H), 3.31 (s, 3H), 2.51-2.41 (m, 1H), 2.38-2.34 (m, 1H).

### Example 62: 1-[(2R,4S)-4-[4-Amino-3-[2-(6-fluoro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one

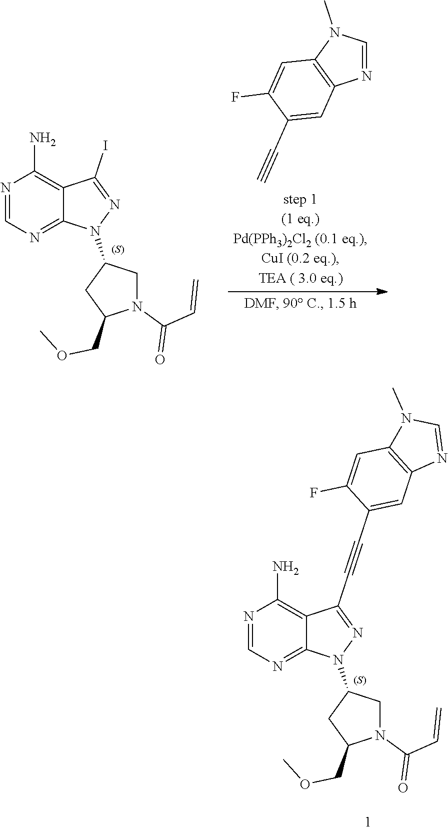

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.20 g, 0.47 mmol), 5-ethynyl-6-fluoro-1-methyl-1,3-benzodiazole (81.35 mg, 0.47 mmol), Pd(PPh₃)₂Cl₂ (32.78 mg, 0.05 mmol) and CuI (17.79 mg, 0.09 mmol) in DMF (2.00 mL) was added TEA (0.19 mL, 1.93 mmol). The reaction mixture was degassed with argon for three times and stirred for 1.5 h at 90° C. The resulting mixture was concentrated under reduced pressure. The residue was purified by silica gel column chromatography, eluted with DCM/MeOH (10/1) to afford the crude product. The crude product was further purified by reverse flash chromatography with the following conditions: Column: SunFire Prep C18 OBD Column, 19×150 mm 5 μm 10 nm; Mobile Phase A: water (0.1% FA), Mobile Phase B: ACN; Flow rate: 20 mL/min; Gradient: 25 B to 50 B in 6 min; 210/254 nm; RT1: 5.56 min. The fractions contained desired product were combined and concentrated to afford 1-[(2R,4S)-4-[4-amino-3-[2-(6-fluoro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one (72.40 mg, 32%) as an off-white solid. MS ESI calculated for C₂₄H₂₃FN₈O₂ [M+H]⁺, 475.19, found 475.25; ¹H NMR (400 MHz, DMSO-d₆): δ 8.48-8.22 (m, 2H), 8.14 (d, J=6.3 Hz, 1H), 7.70 (d, J=9.7 Hz, 1H), 6.68-6.65 (m, 2H), 6.19-6.15 (m, 1H), 5.73-5.42 (m, 2H), 4.55-4.52 (m, 1H), 4.12-4.08 (m, 1H), 3.96-3.92 (m, 1H), 3.85 (s, 3H), 3.67-3.45 (m, 2H), 3.32 (s, 3H), 2.66-2.63 (m, 1H), 2.46-2.32 (m, 1H).

### Example 63: 1-[(2R,4S)-4-[4-Amino-5-[2-(6-fluoro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one

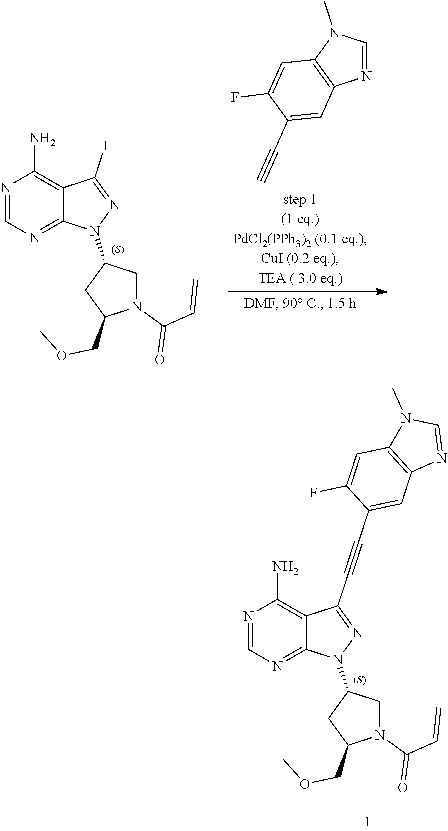

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (0.20 g, 0.47 mmol), 5-ethynyl-6-fluoro-1-methyl-1,3-benzodiazole (81.54 mg, 0.47 mmol), Pd(PPh₃)₂Cl₂ (32.86 mg, 0.05 mmol) and CuI (17.83 mg, 0.09 mmol) in DMF (2.00 mL) was added TEA (0.20 mL, 1.93 mmol). The reaction mixture was degassed with argon for three times and stirred for 1.5 h at 90° C. The resulting mixture was concentrated under reduced pressure. The residue was purified by silica gelcolumn chromatography, eluted with DCM/MeOH (10/1) to afford crude product. The crude product was purified by reverse flash chromatography with the following conditions: column: C18 silica gel; mobile phase: ACN in water (10 mmol/L NH₄HCO₃), 10% to 40% gradient in 40 min; detector: UV 254 nm. The fractions contained desired product were combined and concentrated to afford 1-[(2R,4S)-4-[4-amino-5-[2-(6-fluoro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one (70.70 mg, 31%) as an off-white solid. MS ESI calculated for C₂₅H₂₄FN₇O₂ [M+H]⁺, 474.20, found 474.20; ¹H NMR (400 MHz, DMSO-d₆) δ 8.29 (s, 1H), 8.20-8.17 (m, 1H), 7.92-7.90 (m, 1H), 7.81 (d, J=18.2 Hz, 1H), 7.66 (d, J=9.8 Hz, 1H), 6.78-6.72 (m, 1H), 6.59-6.55 (m, 1H), 6.19-6.16 (m, 1H), 5.72-5.69 (m, 1H), 5.59-5.35 (m, 1H), 4.66-4.36 (m, 1H), 4.13-4.09 (m, 1H), 3.85-3.83 (m, 4H), 3.64-3.44 (m, 2H), 3.33-3.31 (m, 3H), 2.76-2.55 (m, 1H), 2.42-2.30 (m, 1H).

### Example 64: 1-[(2R,4S)-4-(4-Amino-3-[2-[1-(difluoromethyl)-4,6-difluoro-1,3-benzodiazol-5-yl]ethynyl]pyrazolo[3,4-d]pyrimidin-1-yl)-2-(methoxymethyl)pyrrolidin-1-yl]prop-2-en-1-one

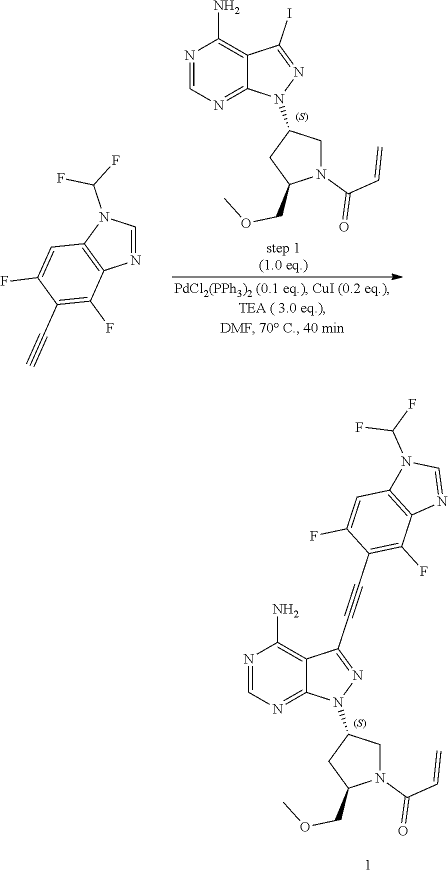

To a stirred mixture of 1-(difluoromethyl)-5-ethynyl-4,6-difluoro-1,3-benzodiazole (80.00 mg, 0.35 mmol), 1-[(2R,4S)-4-[4-amino-3-iodopyrazolo[3,4-d]pyrimidin-1-yl]-2-(methoxymethyl)pyrrolidin-1-yl]prop-2-en-1-one (0.15 g, 0.35 mmol), Pd(PPh₃)₂Cl₂ (24.61 mg, 0.04 mmol) and CuI (13.36 mg, 0.07 mmol) in DMF (4.00 mL) was added TEA (0.11 g, 1.05 mmol) at room temperature. The reaction mixture was degassed with argon for three times and stirred for 40 min at 70° C. The residue was purified by silica gel column chromatography, eluted with DCM/MeOH (10/1) to afford the crude product which was further purified by reverse phase flash with the following conditions: Column: Spherical C18, 20˜40 μm, 330 g; Mobile Phase A: water (10 mmol/L NH₄HCO₃), Mobile Phase B: ACN; Flow rate: 45 mL/min; Gradient (B %): 26%˜36%, 20 min; Detector: 254 nm. The fractions contained desired product were combined and concentrated to afford 1-[(2R,4S)-4-(4-amino-3-[2-[1-(difluoromethyl)-4,6-difluoro-1,3-benzodiazol-5-yl]ethynyl]pyrazolo[3,4-d]pyrimidin-1-yl)-2-(methoxymethyl)pyrrolidin-1-yl]prop-2-en-1-one (0.13 g, 68%) as a white solid. MS ESI calculated for C₂₄H₂₀F₄N₈O₂ [M+H]⁺, 529.17, found 529.10; ¹H NMR (400 MHz, DMSO-d₆) δ 8.80 (s, 1H), 8.31-8.27 (m, 1H), 8.05 (d, J=58.5 Hz, 1H), 7.78 (dd, J=8.5, 1.1 Hz, 1H), 6.75 (dd, J=16.7, 10.3 Hz, 1H), 6.60-6.52 (m, 1H), 6.21-6.17 (m, 1H), 5.74-5.57 (m, 2H), 4.62-4.44 (m, 1H), 4.12-3.79 (m, 2H), 3.66-3.46 (m, 2H), 3.32 (s, 3H), 2.73-2.56 (m, 1H), 2.45-2.32 (m, 1H).

### Example 65: 1-[(2R,4S)-4-(4-Amino-3-[2-[1-(difluoromethyl)-4,6-difluoro-1,3-benzodiazol-5-yl]ethynyl]pyrazolo[4,3-c]pyridin-1-yl)-2-(methoxymethyl)pyrrolidin-1-yl]prop-2-en-1-one

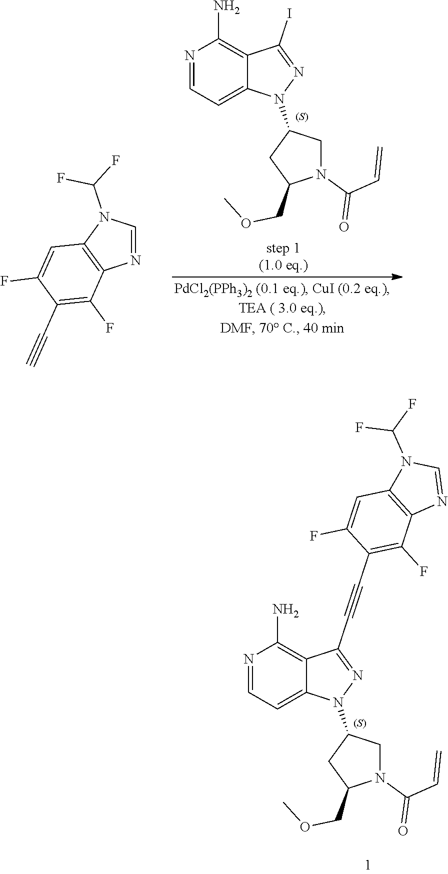

To a stirred mixture of 1-(difluoromethyl)-5-ethynyl-4,6-difluoro-1,3-benzodiazole (53.40 mg, 0.23 mmol), Pd(PPh₃)₂Cl₂ (16.43 mg, 0.02 mmol), CuI (8.92 mg, 0.05 mmol) and 1-[(2R,4S)-4-[4-amino-3-iodopyrazolo[4,3-c]pyridin-1-yl]-2-(methoxymethyl)pyrrolidin-1-yl]prop-2-en-1-one (0.10 g, 0.23 mmol) in DMF (4.00 mL) was added TEA (71.05 mg, 0.70 mmol) at room temperature. The reaction mixture was degassed with argon for three times and stirred for 40 min at 70° C. The resulting mixture was concentrated under reduced pressure. The residue was purified by silica gel column chromatography, eluted with DCM/MeOH (10/1). The fractions contained desired product were combined and concentrated to afford the crude product. Then the crude product was purified by Prep-HPLC with the following condition Column: X-Bridge Prep C18 OBD Column, 19×150 mm 5 μm; Mobile Phase A: water (10 mmol/L NH₄HCO₃, Mobile Phase B: ACN; Flow rate: 20 mL/min; Gradient: 35 B to 60 B in 5.8 min; 210/254 nm; RT1: 5.56 min. The fractions contained desired product were combined and concentrated under reduced pressure to afford 1-[(2R,4S)-4-(4-amino-3-[2-[1-(difluoromethyl)-4,6-difluoro-1,3-benzodiazol-5-yl]ethynyl]pyrazolo[4,3-c]pyridin-1-yl)-2-(methoxymethyl)pyrrolidin-1-yl]prop-2-en-1-one (86.10 mg, 69%) as a white solid. MS ESI calculated for C₂₅H₂₁F₄N₇O₂ [M+H]⁺, 528.17, found 528.09; ¹H NMR (400 MHz, DMSO-d₆) δ 8.81 (s, 1H), 8.15-8.12 (m, 1H), 7.84-7.78 (m, 2H), 7.00-6.99 (m, 1H), 6.79-6.54 (m, 3H), 6.19-6.13 (m, 1H), 5.72-5.68 (m, 1H), 5.55-5.49 (m, 1H), 4.83-4.43 (m, 1H), 4.11-3.78 (m, 2H), 3.68-3.44 (m, 2H), 3.32-3.29 (m, 3H), 2.78-2.54 (m, 1H), 2.41-2.30 (m, 1H).

### Example 66: 1-[(2R,4S)-4-[4-Amino-5-[2-(1-methyl-1,3-benzodiazol-5-yl)ethynyl]pyrrolo[2,3-d]pyrimidin-7-yl]-2-[(trifluoromethoxy)methyl]pyrrolidin-1-yl]prop-2-en-1-one

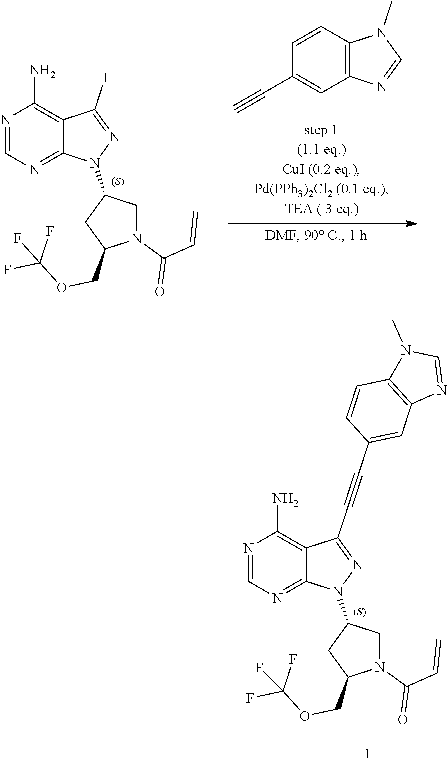

To a stirred mixture of 1-[(2R,4S)-4-[4-amino-5-iodopyrrolo[2,3-d]pyrimidin-7-yl]-2-[(trifluoromethoxy)methyl]pyrrolidin-1-yl]prop-2-en-1-one (0.25 g, 0.52 mmol), 5-ethynyl-1-methyl-1,3-benzodiazole (89.26 mg, 0.57 mmol), Pd(PPh₃)₂Cl₂ (36.46 mg, 0.05 mmol) and CuI (19.79 mg, 0.10 mmol) in DMF (2.50 mL) was added TEA (0.22 mL, 2.14 mmol). The reaction mixture was degassed with argon for three times and stirred for 1 h at 90° C. The resulting mixture was diluted with water (20 mL) and extracted with EA (3×20 mL). The combined organic layers was washed with brine (3×20 mL), dried over anhydrous Na₂SO₄ and filtered. The filtrate was concentrated under reduced pressure. The residue was purified by silica gel column chromatography, eluted with DCM/MeOH (10/1) to afford the crude product. The crude product was purified by Prep-HPLC with the following conditions Column: XBridge Prep Phenyl OBD Column, 19×150 mm 5 μm 13 nm; Mobile Phase A: water (10 mmol/L NH₄HCO₃), Mobile Phase B: ACN; Flow rate: 20 mL/min; Gradient: 30 B to 70 B in 4.3 min; 254/210 nm; RT1: 4.23 min. The fractions contained desired product were combined and concentrated to afford 1-[(2R,4S)-4-[4-amino-5-[2-(1-methyl-1,3-benzodiazol-5-yl)ethynyl]pyrrolo[2,3-d]pyrimidin-7-yl]-2-[(trifluoromethoxy)methyl]pyrrolidin-1-yl]prop-2-en-1-one (38.20 mg, 14%) as an off-white solid. MS ESI calculated for C₂₅H₂₂F₃N₇O₂ [M+H]⁺, 510.18, found 510.20; ¹H NMR (300 MHz, CDCl₃) δ 8.33 (s, 1H), 7.97 (d, J=11.4 Hz, 2H), 7.57-7.35 (m, 2H), 7.20 (s, 1H), 6.53-6.33 (m, 2H), 5.79 (d, J=5.9 Hz, 3H), 5.61-5.50 (m, 1H), 4.75 (s, 1H), 4.52-4.49 (m, 1H), 4.21-4.18 (m, 2H), 3.93-3.89 (m, 4H), 2.76-2.57 (m, 2H).

### Example 67: 1-((2R,4S)-4-(4-Amino-5-((6-fluoro-1-methyl-1H-benzo[d]imidazol-5-yl)ethynyl)-7H-pyrrolo[2,3-d]pyrimidin-7-yl)-2-((trifluoromethoxy)methyl)pyrrolidin-1-yl)prop-2-en-1-one

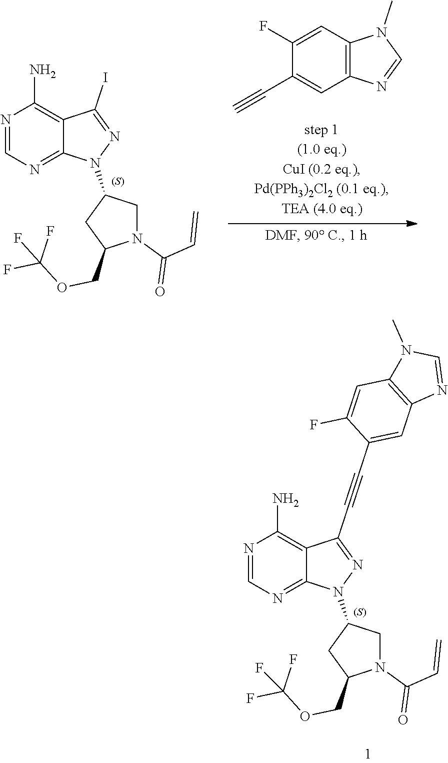

To a stirred mixture of 1-[(2R,4S)-4-[4-amino-5-iodopyrrolo[2,3-d]pyrimidin-7-yl]-2-[(trifluoromethoxy)methyl]pyrrolidin-1-yl]prop-2-en-1-one (0.25 g, 0.52 mmol), 5-ethynyl-6-fluoro-1-methyl-1,3-benzodiazole (90.49 mg, 0.52 mmol), CuI (19.79 mg, 0.11 mmol), Pd(PPh₃)₂Cl₂ (36.46 mg, 0.05 mmol) in DMF (2.50 mL) was added TEA (0.22 mL, 2.14 mmol). The reaction mixture was degassed with argon for three times and stirred for 1 h at 90° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by Prep-HPLC with the following conditions: Column: XBridge Prep Phenyl OBD Column, 19×150 mm 5 μm 13 nm; Mobile Phase A: water (10 mmol/L NH₄HCO₃), Mobile Phase B: ACN; Flow rate: 20 mL/min; Gradient: 30 B to 50 B in 4.3 min; 254/210 nm; RT1: 4.25 min. The fractions contained desired product were combined and concentrated to afford 1-((2R,4S)-4-(4-amino-5-((6-fluoro-1-methyl-1H-benzo[d]imidazol-5-yl)ethynyl)-7H-pyrrolo[2,3-d]pyrimidin-7-yl)-2-((trifluoromethoxy)methyl)pyrrolidin-1-yl)prop-2-en-1-one (41.00 mg, 15%) as an off-white solid. MS ESI calculated for C₂₅H₂₁F₄N₇O₂ [M+H]⁺, 528.17, found 528.20; ¹H NMR (400 MHz, DMSO-d₆) δ 8.30 (s, 1H), 8.19 (s, 1H), 7.94-7.78 (m, 2H), 7.68-7.66 (m, 1H), 6.63-6.56 (m, 2H), 6.24-6.19 (m, 1H), 5.74-5.71 (m, 1H), 5.52-5.38 (m, 1H), 4.85-4.59 (m, 1H), 4.42-4.38 (m, 1H), 4.28-4.13 (m, 2H), 3.93-3.91 (m, 1H), 3.89-3.84 (m, 3H), 2.70-2.67 (m, 1H), 2.51-2.40 (m, 1H).

### Example 68: 1-((2R,4S)-4-(4-Amino-5-((4,6-difluoro-1-methyl-1H-benzo[d]imidazol-5-yl)ethynyl)-7H-pyrrolo[2,3-d]pyrimidin-7-yl)-2-((trifluoromethoxy)methyl)pyrrolidin-1-yl)prop-2-en-1-one

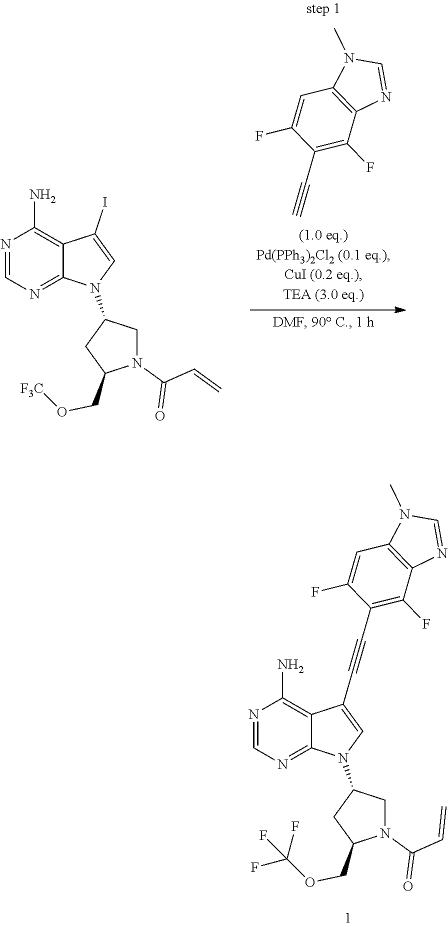 [SMILES: C=CC(=O)N1C[C@@H](n2cc(I)c3c(N)ncnc32)C[C@@H]1COC(F)(F)F ; C#Cc1c(F)cc2c(ncn2C)c1F ; C=CC(=O)N1C[C@@H](n2cc(C#Cc3c(F)cc4c(ncn4C)c3F)c3c(N)ncnc32)C[C@@H]1COC(F)(F)F]

To a stirred mixture of 1-[(2R,4S)-4-[4-amino-5-iodopyrrolo[2,3-d]pyrimidin-7-yl]-2-[(trifluoromethoxy)methyl]pyrrolidin-1-yl]prop-2-en-1-one (0.23 g, 0.48 mmol), 5-ethynyl-4,6-difluoro-1-methyl-1,3-benzodiazole (91.85 mg, 0.48 mmol), CuI (18.21 mg, 0.10 mmol), Pd(PPh₃)₂Cl₂ (33.55 mg, 0.05 mmol) in DMF (2.50 mL) was added TEA (0.22 mL, 2.14 mmol). The reaction mixture was degassed with argon for three times and stirred for 1 h at 90° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by Prep-HPLC with the following conditions: Column: XBridge Prep Phenyl OBD Column, 19×150 mm 5 μm 13 nm; Mobile Phase A: water (10 mmol/L NH₄HCO₃), Mobile Phase B: ACN; Flow rate: 20 mL/min; Gradient: 30 B to 50 B in 4.3 min; 254/210 nm; RT1: 4.25 min. The fractions contained desired product were combined and concentrated to afford 1-((2R,4S)-4-(4-amino-5-((4,6-difluoro-1-methyl-1H-benzo[d]imidazol-5-yl)ethynyl)-7H-pyrrolo[2,3-d]pyrimidin-7-yl)-2-((trifluoromethoxy)methyl)pyrrolidin-1-yl)prop-2-en-1-one (41.00 mg, 15%) as an off-white solid. MS ESI calculated for C₂₅H₂₀F₅N₇O₂ [M+H]⁺, 546.16, found 546.15; ¹H NMR (400 MHz, DMSO-d₆) δ 8.21 (s, 1H), 8.19 (s, 1H), 7.92-7.86 (m, 1H), 7.63-7.60 (m, 1H), 6.63-6.56 (m, 1H), 6.23-6.19 (m, 1H), 5.74-5.71 (m, 1H), 5.54-5.51 (m, 1H), 4.85-4.59 (m, 1H), 4.42-4.38 (m, 1H), 4.28-4.13 (m, 2H), 3.94-3.86 (m, 4H), 2.70-2.67 (m, 1H), 2.51-2.40 (m, 1H).

### Example 69: 1-[(2R,4S)-4-(4-Amino-5-[2-[1-(difluoromethyl)-4,6-difluoro-1,3-benzodiazol-5-yl]ethynyl] pyrrolo[2,3-d]pyrimidin-7-yl)-2-(methoxymethyl)pyrrolidin-1-yl]prop-2-en-1-one

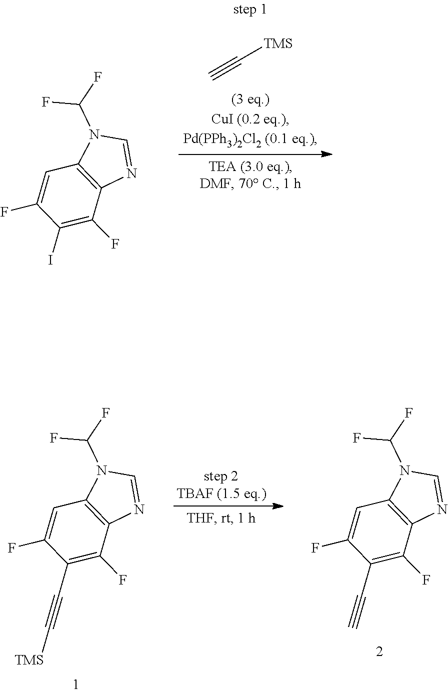

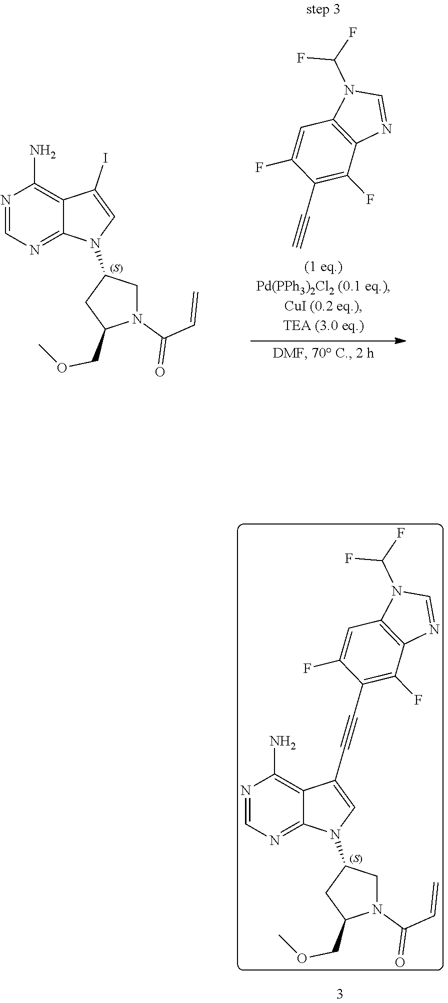

1-[(2R,4S)-4-(4-amino-5-[2-[1-(difluoromethyl)-4,6-difluoro-1,3-benzodiazol-5-yl]ethynyl]pyrrolo[2,3-d]pyrimidin-7-yl)-2-(methoxymethyl)pyrrolidin-1-yl]prop-2-en-1-one. MS ESI calculated for C₂₅H₂₁F₄N₇O₂ [M+H]⁺, 528.27, found 528.10; ¹H NMR (300 MHz, DMSO-d₆) δ 8.76 (s, 1H), 8.31-8.11 (m, 2H), 7.96-7.91 (d, J=8.5 Hz, 1H), 7.74-7.71 (m, 1H), 6.81-6.72 (m, 1H), 6.61-6.52 (m, 1H), 6.20-6.14 (m, 1H), 5.74-5.66 (m, 1H), 5.57-5.43 (m, 1H), 4.61-4.46 (m, 1H), 4.13-4.07 (m, 2H), 3.62-3.45 (m, 2H), 3.43-3.40 (m, 3H), 2.86-2.60 (m, 1H), 2.49-2.35 (m, 1H).

### Example 70: 1-((2R,4S)-4-(4-Amino-3-((1-ethyl-4,6-difluoro-1H-benzo[d]imidazol-5-yl)ethynyl)-1H-pyrazolo[3,4-d]pyrimidin-1-yl)-2-((trifluoromethoxy)methyl)pyrrolidin-1-yl)prop-2-en-1-one

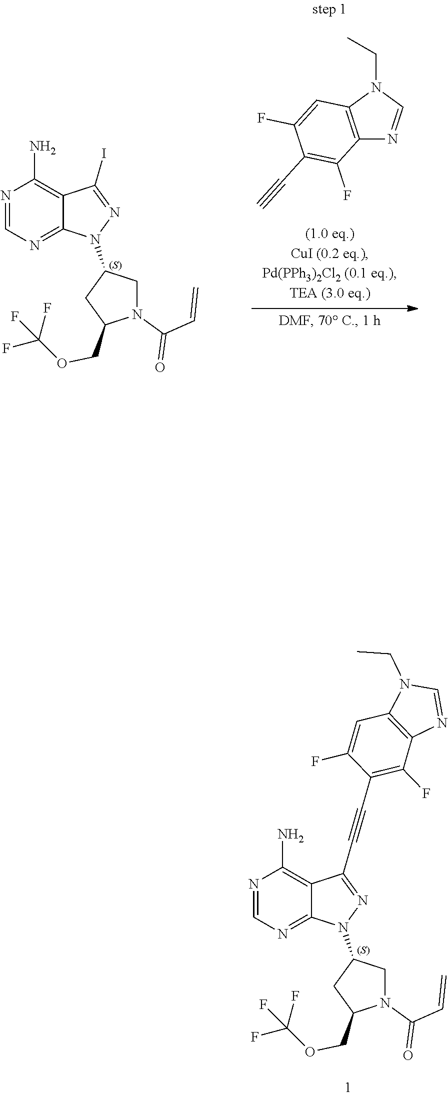

To a stirred mixture of 1-[(2R,4S)-4-[4-amino-3-iodopyrazolo[3,4-d]pyrimidin-1-yl]-2-[(trifluoromethoxy)methyl]pyrrolidin-1-yl]prop-2-en-1-one (0.13 g, 0.27 mmol), 1-ethyl-5-ethynyl-4,6-difluoro-1,3-benzodiazole (55.59 mg, 0.27 mmol), Pd(PPh₃)₂Cl₂ (18.92 mg, 0.02 mmol) and CuI (10.27 mg, 0.05 mmol) in DMF (2.50 mL) was added TEA (81.84 mg, 0.81 mmol). The reaction mixture was degassed with argon for three times and stirred for 1 h at 70° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by silica gel column chromatography, eluted with DCM/MeOH (10/1) to afford the crude product (0.15 g) which was further purified by Prep-HPLC with the following conditions: Column: XBridge Prep C18 OBD Column, 19×150 mm 5 μm; Mobile Phase A: water (10 mmol/L NH₄HCO₃), Mobile Phase B: ACN; Flow rate: 20 mL/min; Gradient: 35 B to 60 B in 6 min; 210/254 nm; RT1: 5.56 min. The fractions contained desired product were combined and concentrated to afford 1-((2R,4S)-4-(4-amino-3-((1-ethyl-4,6-difluoro-1H-benzo[d]imidazol-5-yl)ethynyl)-1H-pyrazolo[3,4-d]pyrimidin-1-yl)-2-((trifluoromethoxy)methyl)pyrrolidin-1-yl)prop-2-en-1-one (74.50 mg, 48%) as an off-white solid. MS ESI calculated for C₂₅H₂₁F₅N₈O₂ [M+H]⁺, 561.48, found 561.00; ¹H NMR (300 MHz, DMSO-d₆) δ 8.48 (s, 1H), 8.33 (s, 1H), 7.74 (d, J=9.2 Hz, 1H), 6.76-6.60 (m, 1H), 6.20 (d, J=16.8 Hz, 1H), 5.87-5.47 (m, 2H), 4.56 (d, J=36.8 Hz, 1H), 4.42-4.23 (m, 3H), 4.10 (d, J=8.7 Hz, 2H), 3.85-3.78 (m, 1H), 2.72-2.60 (m, 1H), 2.49-2.42 (m, 1H), 1.43 (t, J=7.2 Hz, 3H).

### Example 71: 1-[(2R,4S)-4-[4-amino-3-[2-(1-ethyl-6-fluoro-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one

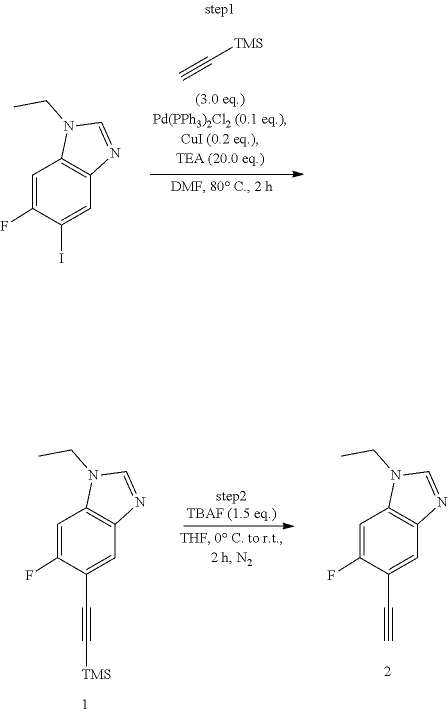

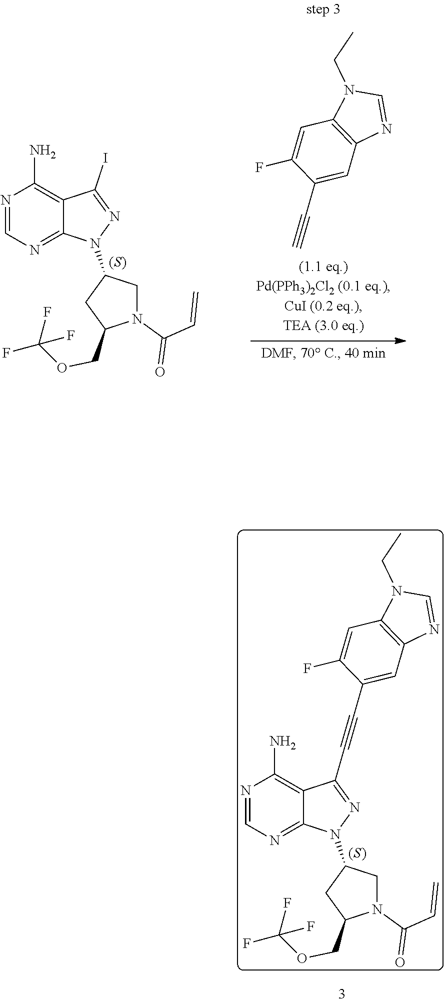

1-[(2R,4S)-4-[4-amino-3-[2-(1-ethyl-6-fluoro-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one. MS ESI calculated for C₂₅H₂₂F₄N₈O₂ [M+H]⁺, 543.18, found 543.25; ¹H NMR (400 MHz, DMSO-d₆): δ 8.41 (s, 1H), 8.30 (s, 1H), 8.06-7.97 (m, 2H), 7.19 (d, J=9.2 Hz, 1H), 6.75-6.55 (m, 2H), 6.17-6.14 (m, 1H), 5.76-5.65 (m, 2H), 4.78 (d, J=7.3 Hz, 1H), 4.49-4.25 (m, 4H), 4.15-4.07 (m, 2H), 2.82-2.67 (m, 1H), 2.43-2.39 (m, 1H), 1.57 (t, J=7.3 Hz, 3H).

### Example 72: 1-[(2R,4S)-4-[4-Amino-3-[2-(1-cyclopropyl-4,6-difluoro-1,3-benzodiazol-5-yl)ethynyl]pyrazolo[3,4-d]pyrimidin-1-yl]-2-(methoxymethyl)pyrrolidin-1-yl]prop-2-en-1-one

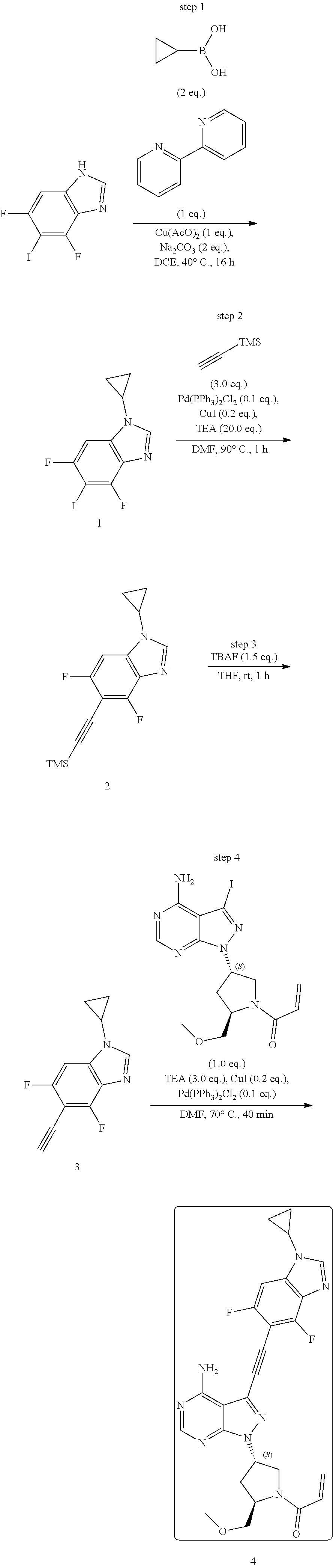

1-[(2R,4S)-4-[4-amino-3-[2-(1-cyclopropyl-4,6-difluoro-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₆H₂₄F₂N₈O₂ [M+H]⁺, 519.20, found 519.10; ¹H NMR (400 MHz, CDCl₃) δ 8.43-8.04 (m, 2H), 6.76-6.41 (m, 2H), 6.27-6.20 (m, 2H), 5.95-5.63 (m, 2H), 4.76-4.48 (m, 1H), 4.26-3.94 (m, 2H), 3.99-3.85 (m, 1H), 3.60-3.42 (m, 6H), 3.07-2.77 (m, 1H), 2.51-2.47 (m, 1H), 1.28-1.23 (m, 2H), 1.12-1.08 (m, 2H).

### Example 73: 1-[(2R,4S)-4-[4-Amino-3-[2-(1-cyclopropyl-4,6-difluoro-1,3-benzodiazol-5-yl)ethynyl]pyrazolo[4,3-c]pyridin-1-yl]-2-(methoxymethyl)pyrrolidin-1-yl]prop-2-en-1-one; formic acid

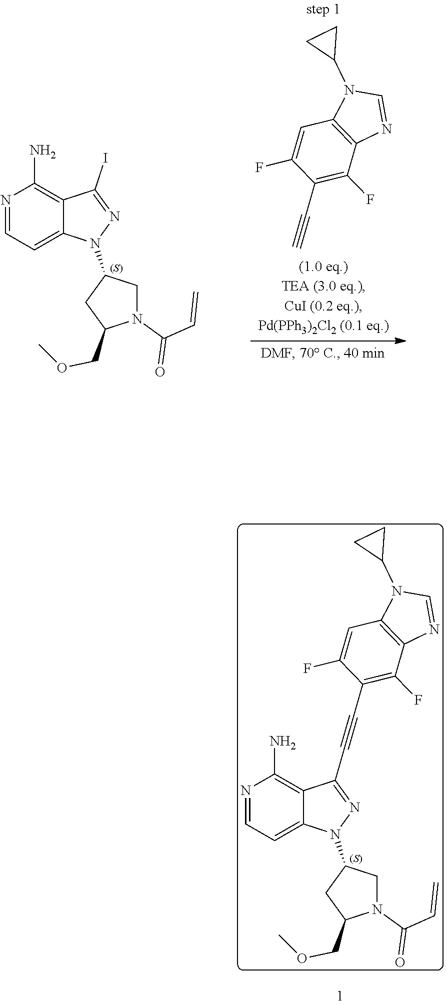

To a stirred mixture of 1-cyclopropyl-5-ethynyl-4,6-difluoro-1,3-benzodiazole (51.31 mg, 0.23 mmol), 1-[(2R,4S)-4-[4-amino-3-iodopyrazolo[4,3-c]pyridin-1-yl]-2-propylpyrrolidin-1-yl]prop-2-en-1-one (0.10 g, 0.23 mmol), Pd(PPh₃)₂Cl₂ (16.50 mg, 0.02 mmol) and CuI (8.96 mg, 0.05 mmol) in DMF (2.00 mL) was added TEA (71.38 mg, 0.70 mmol). The reaction mixture was degassed with nitrogen for three times and stirred for 40 min at 70° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by silica gel column chromatography, eluted with DCM MeOH (10/1) to afford the crude product. The crude product was purified by Prep-HPLC with the following conditions Column: Sun-Fire Prep C18 OBD Column, 19×150 mm 5 μm 10 nm; Mobile Phase A: water (0.1% FA), Mobile Phase B: ACN; Flow rate: 20 mL/min; Gradient: 35 B to 50 B in 6 min; 210/254 nm; RT: 5.36 min. The fractions contained desired product were combined and concentrated under reduced pressure to afford 1-[(2R,4S)-4-[4-amino-3-[2-(1-cyclopropyl-4,6-difluoro-1,3-benzodiazol-5-yl)ethynyl]pyrazolo[4,3-c]pyridin-1-yl]-2-(methoxymethyl)pyrrolidin-1-yl]prop-2-en-1-one; formic acid (40.20 mg, 30%) as a white solid. MS ESI calculated for C₂₈H₂₇F₂N₇O₄ [M+H−46]⁺, 518.20, found 518.15; ¹H NMR (400 MHz, CDCl₃) δ 7.97 (s, 1H), 7.70-7.53 (m, 2H), 7.19 (d, J=8.2 Hz, 1H), 6.76-6.72 (m, 1H), 6.45-6.42 (m, 2H), 5.79-5.68 (m, 1H), 5.56-5.30 (m, 1H), 4.65-4.52 (m, 1H), 4.17-4.08 (m, 2H), 3.93-3.90 (m, 1H), 3.55 (d, J=4.0 Hz, 1H), 3.49-3.45 (m, 1H), 3.43-3.34 (m, 4H), 2.80-2.69 (m, 1H), 2.58-2.39 (m, 1H), 1.28-1.20 (m, 2H), 1.12-1.05 (m, 2H).

### Example 74: 1-[(2R,4S)-4-[4-Amino-3-[2-(6-fluoro-1-methyl-1,3-benzodiazol-5-yl)ethynyl]pyrazolo[4,3-c]pyridin-1-yl]-2-(methoxymethyl)pyrrolidin-1-yl]prop-2-en-1-one

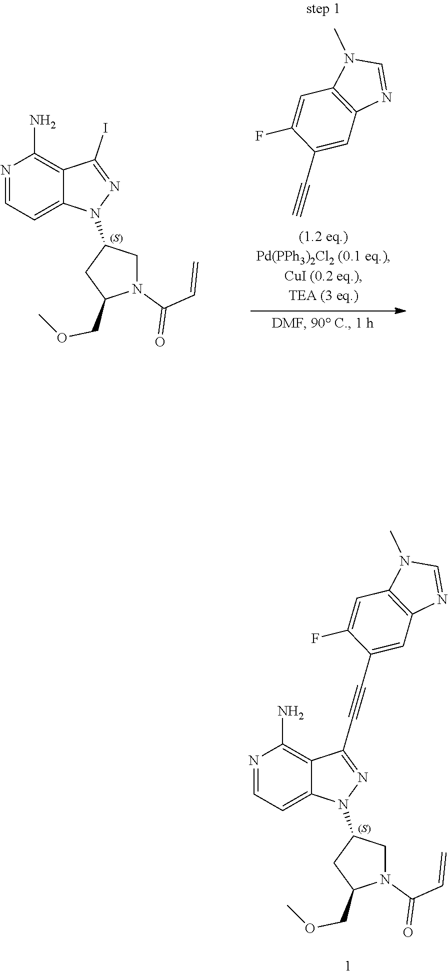

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (2.00 g, 4.68 mmol), 5-ethynyl-6-fluoro-1-methyl-1,3-benzodiazole (0.98 g, 5.62 mmol), Pd(PPh₃)₂Cl₂ (0.33 g, 0.47 mmol) and CuI (0.18 g, 0.94 mmol) in DMF (20.00 mL) was added TEA (2.00 mL, 14.41 mmol) at ambient temperature. The reaction mixture was degassed with argon for three times and stirred for 1 h at 90° C. The resulting mixture was diluted with water (200 mL) and extracted with DCM (4×250 mL). The combined organic layers was washed with brine (5×100 mL), dried over anhydrous Na₂SO₄, filtered and concentrated under reduced pressure. The residue was purified by silica gel column chromatography, eluted with DCM/MeOH (10/1) to afford the crude product. The crude product was purified by trituration with ACN (25 mL). The solids were collected by filtration, washed with ACN (3×10 mL) and dried to afford 1-[(2R,4S)-4-[4-amino-3-[2-(6-fluoro-1-methyl-1,3-benzodiazol-5-yl)ethynyl]pyrazolo[4,3-c]pyridin-1-yl]-2-(methoxymethyl)pyrrolidin-1-yl]prop-2-en-1-one (1.07 g, 48%) as an off-white solid. MS ESI calculated for C₂₅H₂₄FN₇O₂ [M+H]⁺, 474.20, found 474.35; ¹H NMR (400 MHz, DMSO-d₆) δ 8.33 (s, 1H), 8.07 (d, J=6.0 Hz, 1H), 7.81 (d, J=6.8 Hz, 1H), 7.76 (d, J=10.0 Hz, 1H), 7.02 (s, 1H), 6.76-6.53 (m, 3H), 6.18-6.13 (m, 1H), 5.72-5.65 (m, 1H), 5.51 (s, 1H), 4.60-4.47 (m, 1H), 4.09-4.08 (m, 1H), 3.92-3.81 (m, 4H), 3.60-3.48 (m, 2H), 3.32 (s, 3H), 2.50-2.33 (m, 2H).

### Example 75: 1-[(2R,4S)-4-[4-Amino-5-[2-(1-cyclopropyl-4,6-difluoro-1,3-benzodiazol-5-yl)ethynyl]pyrrolo[2,3-d]pyrimidin-7-yl]-2-(methoxymethyl)pyrrolidin-1-yl]prop-2-en-1-one

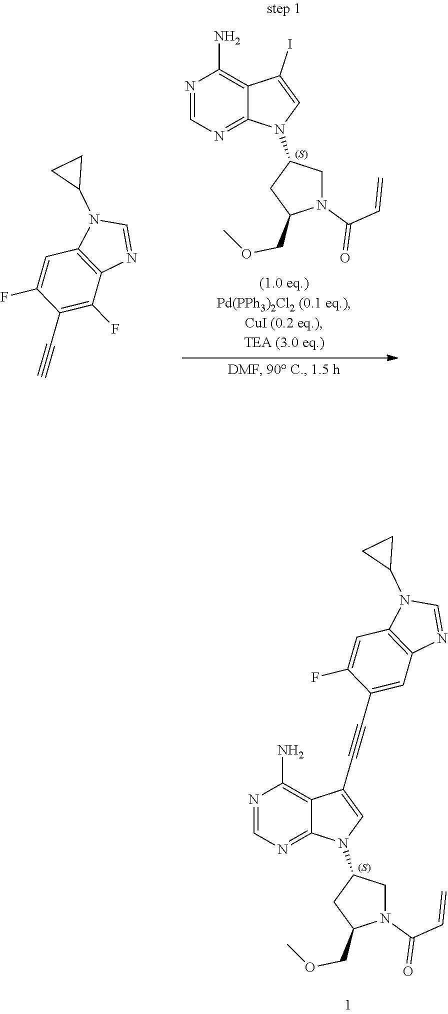

To a stirred mixture of 1-cyclopropyl-5-ethynyl-4,6-difluoro-1,3-benzodiazole (0.15 g, 0.69 mmol) and 1-[(2R,4S)-4-[4-amino-5-iodopyrrolo[2,3-d]pyrimidin-7-yl]-2-(methoxymethyl)pyrrolidin-1-yl]prop-2-en-1-one (0.29 g, 0.69 mmol) in DMF (3.00 mL) were added Pd(PPh₃)₂Cl₂ (48.25 mg, 0.07 mmol), CuI (26.18 mg, 0.14 mmol) and TEA (0.21 g, 2.06 mmol). The reaction mixture was degassed with argon for three times and stirred for 1.5 h at 90° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by silica gel column chromatography, eluted with CH₂Cl₂/MeOH (10/1), the fractions contained desired product were combined and concentrated. The crude product (0.18 g) was purified by Prep-HPLC with the following conditions: Column: SunFire Prep C18 OBD Column, 19×150 mm 5 m 10 nm; Mobile Phase A: water (0.1% FA), Mobile Phase B: ACN; Flow rate: 20 mL/min; Gradient: 35% B to 50% B in 6 min; 210/254 nm; RT: 5.58 min. The fractions contained desired product were combined and concentrated to afford 1-[(2R,4S)-4-[4-amino-5-[2-(1-cyclopropyl-4,6-difluoro-1,3-benzodiazol-5-yl)ethynyl]pyrrolo[2,3-d]pyrimidin-7-yl]-2-(methoxymethyl)pyrrolidin-1-yl]prop-2-en-1-one (75.50 mg, 21%) as a light yellow solid. MS ESI calculated for C₂₇H₂₅F₂N₇O₂ [M+H]⁺, 518.20, found 518.25; ¹H NMR (300 MHz, CDCl₃) δ 8.30 (s, 1H), 7.97 (s, 1H), 7.38 (d, J=6.5 Hz, 1H), 7.19 (d, J=8.4 Hz, 1H), 6.60-6.33 (m, 4H), 5.90-5.40 (m, 2H), 4.68-4.37 (m, 1H), 4.28-4.17 (m, 1H), 3.86-3.52 (m, 3H), 3.44-3.41 (m, 4H), 2.64-2.51 (m, 2H), 1.29-1.25 (m, 2H), 1.14-1.04 (m, 2H).

### Example 76: 1-[(2R,4S)-4-[4-Amino-3-[2-(1-cyclopropyl-4,6-difluoro-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one

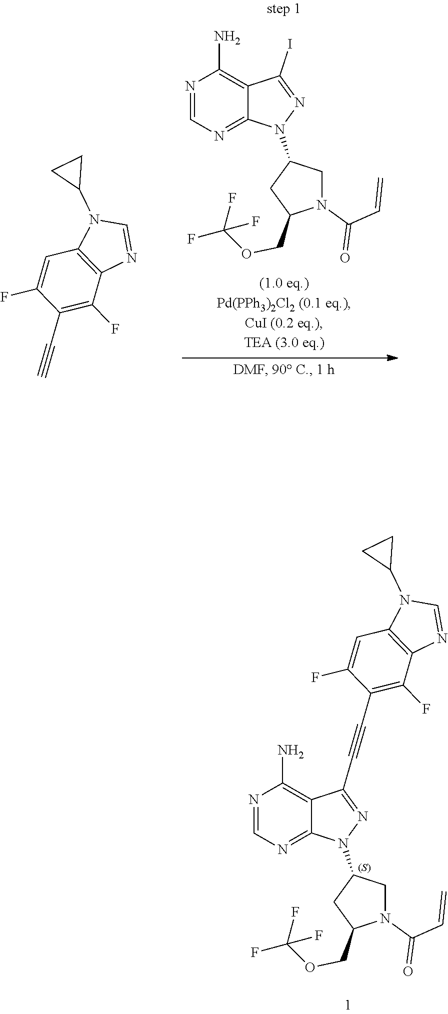

To a stirred mixture of 1-cyclopropyl-5-ethynyl-4,6-difluoro-1,3-benzodiazole (0.15 g, 0.69 mmol) and 1-[(2R,4S)-4-[4-amino-3-iodopyrazolo[3,4-d]pyrimidin-1-yl]-2-[(trifluoromethoxy)methyl]pyrrolidin-1-yl]prop-2-en-1-one (0.33 g, 0.69 mmol) in DMF (3.00 mL) were added Pd(PPh₃)₂Cl₂ (48.25 mg, 0.07 mmol), CuI (26.18 mg, 0.14 mmol) and TEA (0.21 g, 2.06 mmol). The reaction mixture was degassed with argon for three times and stirred for 1 h at 90° C. The resulting mixture was diluted with water (20 mL), extracted with EA (3×20 mL). The combined organic layers was washed with brine (3×20 mL), dried over anhydrous Na₂SO₄ and filtered. The filtrate was concentrated under reduced pressure. The residue was purified by silica gel column chromatography, eluted with CH₂Cl₂/MeOH (10/1), the fractions contained desired product were combined and concentrated. The crude product (0.18 g) was purified by Prep-HPLC with the following conditions: Column: SunFire Prep C18 OBD Column, 19×150 mm 5 μm 10 nm; Mobile Phase A: water (0.1% FA), Mobile Phase B: ACN; Flow rate: 20 mL/min; Gradient: 35% B to 50% B in 6 min; 210/254 nm; RT: 5.58 min. The fractions contained desired product were combined and concentrated to afford 1-[(2R,4S)-4-[4-amino-3-[2-(1-cyclopropyl-4,6-difluoro-1,3-benzodiazol-5-yl)ethynyl]pyrazolo[3,4-d]pyrimidin-1-yl]-2-[(trifluoromethoxy)methyl]pyrrolidin-1-yl]prop-2-en-1-one (60.5 mg, 15%) as a light yellow solid. MS ESI calculated for C₂₆H₂₁F₅N₈O₂ [M+H]⁺, 573.17, found 573.20; ¹H NMR (300 MHz, CDCl₃) δ 8.41 (s, 1H), 8.00 (s, 1H), 7.22 (d, J=8.4 Hz, 1H), 6.43 (d, J=6.1 Hz, 2H), 6.14 (s, 2H), 5.81-5.60 (m, 2H), 4.79 (s, 1H), 4.58-4.45 (m, 1H), 4.16 (d, J=6.5 Hz, 3H), 3.45-3.40 (m, 1H), 3.08-2.96 (m, 1H), 2.52-2.48 (m, 1H), 1.28-1.24 (m, 2H), 1.14-1.11 (m, 2H).

### Example 77: 1-[(2R,4S)-4-[4-Amino-3-[2-(1-ethyl-6-fluoro-1,3-benzodiazol-5-yl)ethynyl]pyrazolo[3,4-d]pyrimidin-1-yl]-2-(methoxymethyl)pyrrolidin-1-yl]prop-2-en-1-one

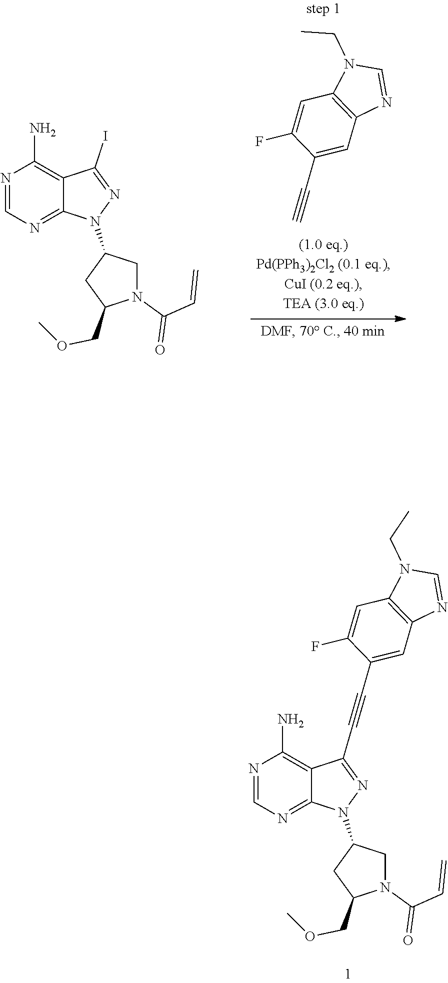 [SMILES: C=CC(=O)N1C[C@@H](n2nc(I)c3c(N)ncnc32)C[C@@H]1COC ; C#Cc1cc2ncn(CC)c2cc1F ; C=CC(=O)N1C[C@@H](n2nc(C#Cc3cc4ncn(CC)c4cc3F)c3c(N)ncnc32)C[C@@H]1COC]

To a stirred solution of 1-[(2R,4S)-4-[4-amino-3-iodopyrazolo[3,4-d]pyrimidin-1-yl]-2-(methoxymethyl)pyrrolidin-1-yl]prop-2-en-1-one (2.00 g, 4.67 mmol), 1-ethyl-5-ethynyl-6-fluoro-1,3-benzodiazole (0.88 g, 4.67 mmol), Pd(PPh₃)₂Cl₂ (0.33 g, 0.46 mmol) and CuI (0.18 g, 0.93 mmol) in DMF (40.00 mL) was added TEA (1.42 g, 14.01 mmol). The reaction mixture was degassed with argon for three times and stirred for 40 min at 70° C. The resulting mixture was diluted with water (200 mL), extracted with EA (3×200 mL). The combined organic layers was washed with brine (3×200 mL), dried over anhydrous Na₂SO₄ and filtered. The filtrate was concentrated under reduced pressure. The residue was purified by silica gel column chromatography, eluted with DCM/MeOH (10/1). The fractions contained desired product were combined and concentrated. Then the crude product was purified by Prep-HPLC with the following conditions: Column: SunFire Prep C18 OBD Column, 19×150 mm 5 μm 10 nm; Mobile Phase A: water (10 mmol/L NH₄HCO₃), Mobile Phase B: ACN; Flow rate: 20 mL/min; Gradient: 25 B to 40 B in 30 min; 220/254 nm. The fractions contained desired product were combined and concentrated to afford 1-[(2R,4S)-4-[4-amino-3-[2-(1-ethyl-6-fluoro-1,3-benzodiazol-5-yl)ethynyl]pyrazolo[3,4-d]pyrimidin-1-yl]-2-(methoxymethyl)pyrrolidin-1-yl]prop-2-en-1-one (1.58 g, 69%) as a white solid. MS ESI calculated for C₂₅H₂₅FN₈O₂ [M+H]⁺, 489.21, found 489.10; ¹H NMR (400 MHz, CDCl₃) δ 8.36 (d, J=5.8 Hz, 1H), 8.10-7.93 (m, 2H), 7.19 (d, J=9.2 Hz, 1H), 6.67-6.37 (m, 2H), 6.26 (s, 2H), 5.88-5.80 (m, 1H), 5.74-5.67 (m, 1H), 4.75-4.45 (m, 1H), 4.26-3.98 (m, 4H), 3.84-3.81 (m, 1H), 3.66-3.47 (m, 1H), 3.40 (s, 3H), 3.00-2.76 (m, 1H), 2.63-2.46 (m, 1H), 1.57-1.55 (m, 3H).

### Example 78: 1-((2R,4S)-4-(4-Amino-3-((1-cyclopropyl-6-fluoro-1H-benzo[d]imidazol-5-yl)ethynyl)-1H-pyrazolo[3,4-d]pyrimidin-1-yl)-2-((trifluoromethoxy)methyl)pyrrolidin-1-yl)prop-2-en-1-one

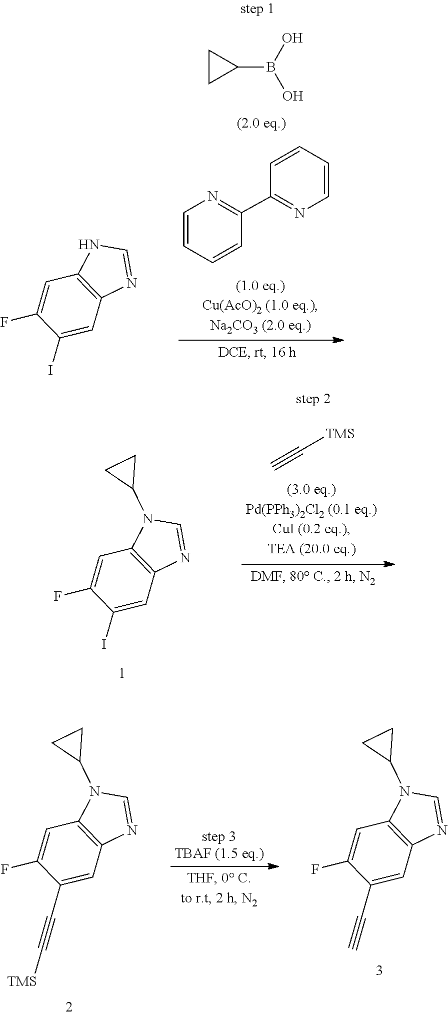

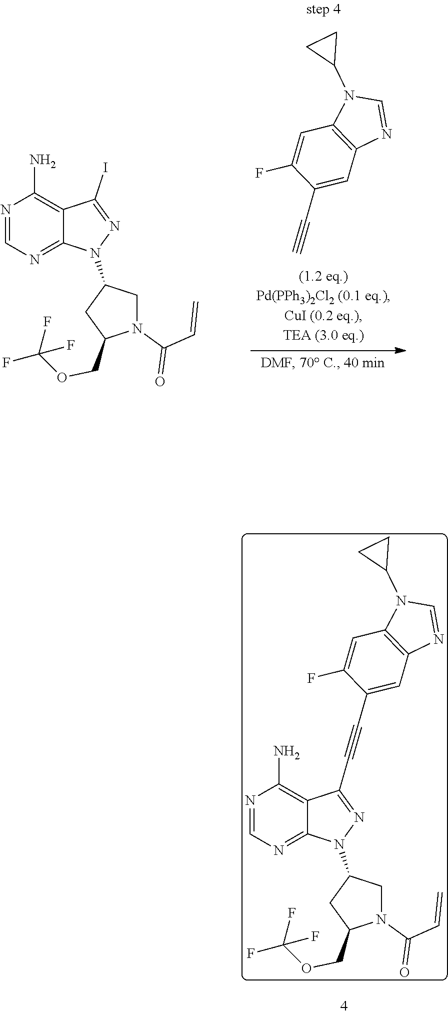 [SMILES: C=CC(=O)N1C[C@@H](n2nc(I)c3c(N)ncnc32)C[C@@H]1COC(F)(F)F ; C#Cc1cc2ncn(C3CC3)c2cc1F ; C=CC(=O)N1C[C@@H](n2nc(C#Cc3cc4ncn(C5CC5)c4cc3F)c3c(N)ncnc32)C[C@@H]1COC(F)(F)F]

1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-((trifluoromethoxy)methyl)pyrrolidin-1-yl)prop-2-en-1-one. MS ESI calculated for C₂₆H₂₂F₄N₈O₂ [M+H]⁺, 555.18, found 555.15; ¹H NMR (400 MHz, DMSO-d₆) δ 8.33 (d, J=28.1 Hz, 2H), 8.16 (d, J=6.1 Hz, 1H), 7.70-7.55 (m, 2H), 6.86-6.50 (m, 2H), 6.19 (d, J=16.8 Hz, 1H), 5.83-5.53 (m, 2H), 4.86-4.73 (m, 1H), 4.48-4.45 (m, 1H), 4.36-4.20 (m, 1H), 4.17-3.97 (m, 2H), 3.55-3.50 (m, 1H), 2.74-2.66 (m, 1H), 2.49-2.37 (m, 1H), 1.21-0.97 (m, 4H).

### Example 79: 1-[(2R,4S)-4-[4-Amino-3-[2-(1-ethyl-6-fluoro-1,3-benzodiazol-5-yl)ethynyl]pyrazolo[4,3-c]pyridin-1-yl]-2-(methoxymethyl)pyrrolidin-1-yl]prop-2-en-1-one

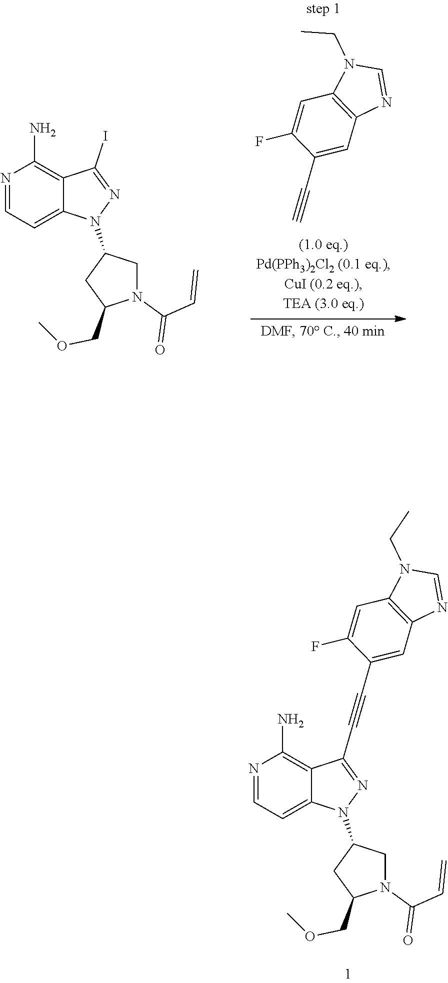 [SMILES: C=CC(=O)N1C[C@@H](n2nc(I)c3c(N)nccc32)C[C@@H]1COC ; C#Cc1cc2ncn(CC)c2cc1F ; C=CC(=O)N1C[C@@H](n2nc(C#Cc3cc4ncn(CC)c4cc3F)c3c(N)nccc32)C[C@@H]1COC]

To a solution of 1-[(2R,4S)-4-[4-amino-3-iodopyrazolo[4,3-c]pyridin-1-yl]-2-(methoxymethyl)pyrrolidin-1-yl]prop-2-en-1-one (4.00 g, 9.36 mmol), 1-ethyl-5-ethynyl-6-fluoro-1,3-benzodiazole (1.76 g, 9.36 mmol), Pd(PPh₃)₂Cl₂ (0.66 g, 0.93 mmol), CuI (0.36 g, 1.87 mmol) in DMF (80.00 mL) was added TEA (2.84 g, 28.08 mmol) dropwise at ambient temperature. The reaction mixture was degassed with argon for three times and stirred for 40 min at 70° C. The resulting mixture was diluted with water (300 mL), extracted with EA (3×300 mL). The combined organic layers was washed with brine (3×200 mL), dried over anhydrous Na₂SO₄ and filtered. The filtrate was concentrated under reduced pressure. The residue was purified by silica gel column chromatography, eluted with DCM/MeOH (10/1), the fractions contained desired product were combined and concentrated. The crude product was purified by reverse phase flash with the following conditions: Column: Spherical C18, 20-40 um, 330 g; Mobile Phase A: water (10 mmol/L NH₄HCO₃), Mobile Phase B: ACN; Flow rate: 100 mL/min; Gradient (B %): 20%-40% within 40 min, Detector: UV 254/220 nm; RT: 40 min. The fractions contained desired product were combined and concentrated to afford 1-[(2R,4S)-4-[4-amino-3-[2-(1-ethyl-6-fluoro-1,3-benzodiazol-5-yl)ethynyl]pyrazolo[4,3-c]pyridin-1-yl]-2-(methoxymethyl)pyrrolidin-1-yl]prop-2-en-1-one (1.13 g, 24%) as a white solid. MS ESI calculated for C₂₆H₂₆FN₇O₂ [M+H]⁺, 488.21, found 488.10; ¹H NMR (400 MHz, CDCl₃) δ 8.06-7.99 (m, 2H), 7.89 (t, J=7.1 Hz, 1H), 7.19 (dd, J=9.2, 1.7 Hz, 1H), 6.74 (d, J=6.2 Hz, 1H), 6.58-6.41 (m, 2H), 5.84-5.71 (m, 3H), 5.57-5.31 (m, 1H), 4.68-4.47 (m, 1H), 4.27-4.09 (m, 4H), 3.92-3.89 (m, 1H), 3.57-3.47 (m, 1H), 3.43 (s, 3H), 3.06-2.67 (m, 1H), 2.60-2.40 (m, 1H), 1.58 (t, J=7.3 Hz, 3H).

### Example 80: 1-[(2R,4S)-4-[4-Amino-5-[2-(1-ethyl-6-fluoro-1,3-benzodiazol-5-yl)ethynyl]pyrrolo[2,3-d]pyrimidin-7-yl]-2-(methoxymethyl)pyrrolidin-1-yl]prop-2-en-1-one

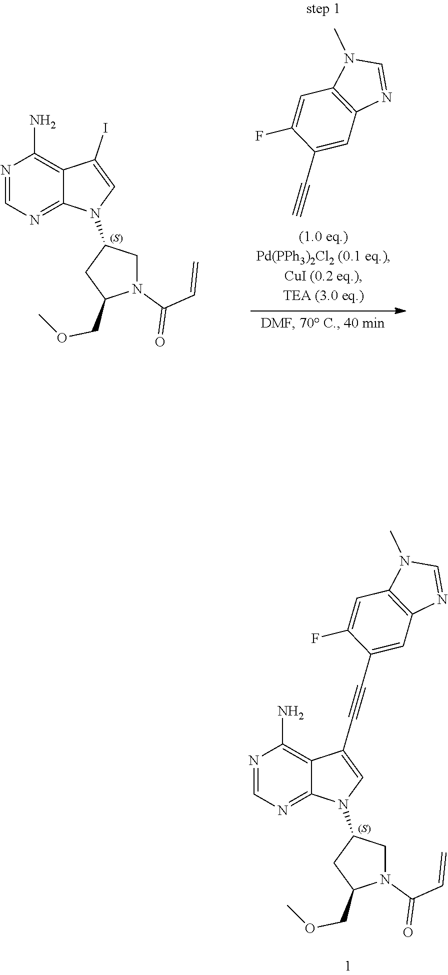

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (1.80 g, 4.21 mmol) and 1-ethyl-5-ethynyl-6-fluoro-1,3-benzodiazole (0.79 g, 4.21 mmol) in DMF (40.00 mL) were added Pd(PPh₃)₂Cl₂ (0.30 g, 0.42 mmol), CuI (0.16 g, 0.84 mmol) and TEA (1.28 g, 12.64 mmol). The reaction mixture was degassed with argon for three times and stirred for 40 min at 70° C. The resulting mixture was allowed to cool down to room temperature. The resulting mixture was diluted with water (200 mL) and extracted with EA (3×200 mL). The combined organic layers was washed with brine (3×200 mL), dried over anhydrous Na₂SO₄ and filtered. The filtrate was concentrated under reduced pressure. The residue was purified by silica gel column chromatography, eluted with CH₂Cl₂/MeOH (10/1), the fractions contained desired product were combined and concentrated. The residue was purified by reverse flash chromatography with the following conditions: column: C18 silica gel; mobile phase: ACN in water (10 mmol/L NH₄HCO₃), 10% to 50% gradient in 30 min; detector: UV 254 nm. The fractions contained desired product were combined and concentrated to afford 1-[(2R,4S)-4-[4-amino-5-[2-(1-ethyl-6-fluoro-1,3-benzodiazol-5-yl)ethynyl]pyrrolo[2,3-d]pyrimidin-7-yl]-2-(methoxymethyl)pyrrolidin-1-yl]prop-2-en-1-one (1.13 g, 54%) as a light yellow solid. MS ESI calculated for C₂₆H₂₆FN₇O₂ [M+H]⁺, 488.21, found 488.30; ¹H NMR (400 MHz, CDCl₃) δ 8.33 (s, 1H), 8.00-7.81 (m, 2H), 7.28 (d, J=8.1 Hz, 1H), 7.17 (d, J=9.1 Hz, 1H), 6.55-6.35 (m, 2H), 5.95 (s, 2H), 5.83-5.51 (m, 2H), 4.71-4.37 (m, 1H), 4.27-4.03 (m, 3H), 3.87-3.49 (m, 3H), 3.41 (s, 3H), 2.64-2.48 (m, 2H), 1.58 (t, J=7.3 Hz, 3H).

### Example 81: 1-[(2R,4S)-4-[4-Amino-3-[2-(1-ethyl-6-fluoro-1,3-benzodiazol-5-yl)ethynyl]pyrazolo[4,3-c]pyridin-1-yl]-2-[(trifluoromethoxy)methyl]pyrrolidin-1-yl]prop-2-en-1-one

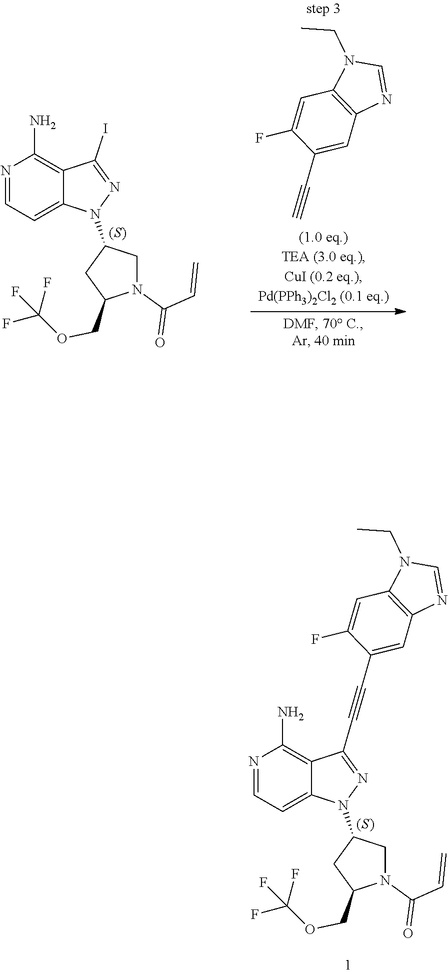

To a stirred solution of 1-[(2R,4S)-4-[4-amino-3-iodopyrazolo[4,3-c]pyridin-1-yl]-2-[(trifluoromethoxy)methyl]pyrrolidin-1-yl]prop-2-en-1-one (4.00 g, 8.31 mmol), 1-ethyl-5-ethynyl-6-fluoro-1,3-benzodiazole (1.56 g, 8.31 mmol), Pd(PPh₃)₂Cl₂ (0.58 g, 0.83 mmol) and CuI (0.32 g, 1.66 mmol) in DMF (70.00 mL) was added TEA (2.52 g, 24.93 mmol). The reaction mixture was degassed with argon for three times and stirred for 40 min at 70° C. The resulting mixture was diluted with water (200 mL), extracted with EA (3×200 mL). The combined organic layers was washed with brine (3×200 mL), dried over anhydrous Na₂SO₄ and filtered. The filtrate was concentrated under reduced pressure. The residue was purified by silica gel column chromatography, eluted with DCM/MeOH (10/1), the fractions contained desired product were combined and concentrated. Then the crude product was purified by Prep-HPLC with the following conditions: Column: Sun-Fire Prep C18 OBD Column, 19×150 mm 5 m 10 nm; Mobile Phase A: water (10 mmoL/L NH₄HCO₃), Mobile Phase B: ACN; Flow rate: 20 mL/min; Gradient: 25 B to 45 B in 30 min; 220/254 nm. The fractions contained desired product were combined and concentrated to afford 1-[(2R,4S)-4-[4-amino-3-[2-(1-ethyl-6-fluoro-1,3-benzodiazol-5-yl)ethynyl]pyrazolo[4,3-c]pyridin-1-yl]-2-[(trifluoromethoxy)methyl]pyrrolidin-1-yl]prop-2-en-1-one (1.17 g, 26%) as a white solid. MS ESI calculated for C₂₆H₂₃F₄N₇O₂ [M+H]⁺, 542.18, found 542.10; ¹H NMR (400 MHz, CDCl₃) δ 8.10-7.94 (m, 2H), 7.89 (d, J=6.3 Hz, 1H), 7.18 (d, J=9.2 Hz, 1H), 6.68 (d, J=6.2 Hz, 1H), 6.55-6.36 (m, 2H), 5.88 (s, 2H), 5.80-5.73 (m, 1H), 5.40-5.32 (m, 1H), 4.78 (d, J=9.0 Hz, 1H), 4.62-4.59 (m, 1H), 4.35-4.06 (m, 5H), 2.92-2.84 (m, 1H), 2.51-2.45 (m, 1H), 1.59-1.55 (m, 3H).

### Example 82: 1-[(2R,4S)-4-(4-Amino-3-[2-[1-(difluoromethyl)-6-fluoro-1,3-benzodiazol-5-yl]ethynyl]pyrazolo[3,4-d]pyrimidin-1-yl)-2-(methoxymethyl)pyrrolidin-1-yl]prop-2-en-1-one

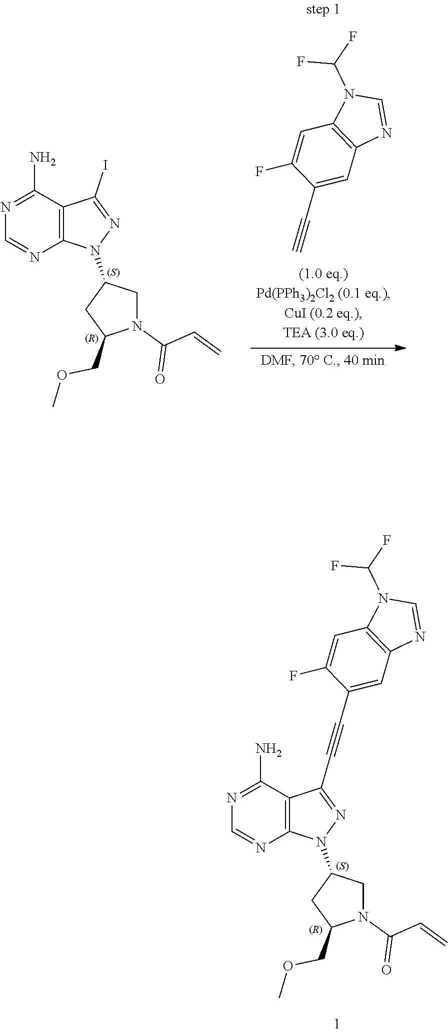

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.40 g, 0.93 mmol) and 1-(difluoromethyl)-5-ethynyl-6-fluoro-1,3-benzodiazole (0.20 g, 0.93 mmol) in DMF (8.00 mL) were added Pd(PPh₃)₂Cl₂ (65.56 mg, 0.09 mmol), CuI (35.58 mg, 0.18 mmol) and TEA (0.28 g, 2.80 mmol) at room temperature. The reaction mixture was degassed with argon for three times and stirred for 40 min at 70° C. The resulting mixture was concentrated under reduced pressure. The residue was purified by silica gel column chromatography, eluted with DCM/MeOH (10/1) to afford crude product. The crude product was purified by reverse flash chromatography with the following conditions: column: C18 silica gel; mobile phase: ACN in water (10 mmol/L NH₄HCO₃), 0% to 45% gradient in 10 min; detector: UV 254 nm. The fractions contained desired product were combined and concentrated to afford 1-[(2R,4S)-4-(4-amino-3-[2-[1-(difluoromethyl)-6-fluoro-1,3-benzodiazol-5-yl]ethynyl]pyrazolo[3,4-d]pyrimidin-1-yl)-2-(methoxymethyl)pyrrolidin-1-yl]prop-2-en-1-one (0.30 g, 62%) as an off-white solid. MS ESI calculated for C₂₄H₂₁F₃N₈O₂ [M+H]⁺, 511.18, found 511.10; ¹H NMR (300 MHz, CDCl₃) δ 8.40 (d, J=4.2 Hz, 1H), 8.19 (s, 1H), 8.10-8.07 (m, 1H), 7.64-7.33 (m, 2H), 6.68-6.37 (m, 2H), 6.21 (s, 2H), 5.96-5.63 (m, 2H), 4.65-4.45 (m, 1H), 4.15-4.02 (m, 2H), 3.94-3.48 (m, 2H), 3.42 (s, 3H), 3.07-2.71 (m, 1H), 2.53-2.48 (m, 1H).

### Example 83: 1-[(2R,4S)-4-(4-Amino-3-[2-[1-(difluoromethyl)-6-fluoro-1,3-benzodiazol-5-yl]ethynyl]pyrazolo[4,3-c]pyridin-1-yl)-2-(methoxymethyl)pyrrolidin-1-yl]prop-2-en-1-one

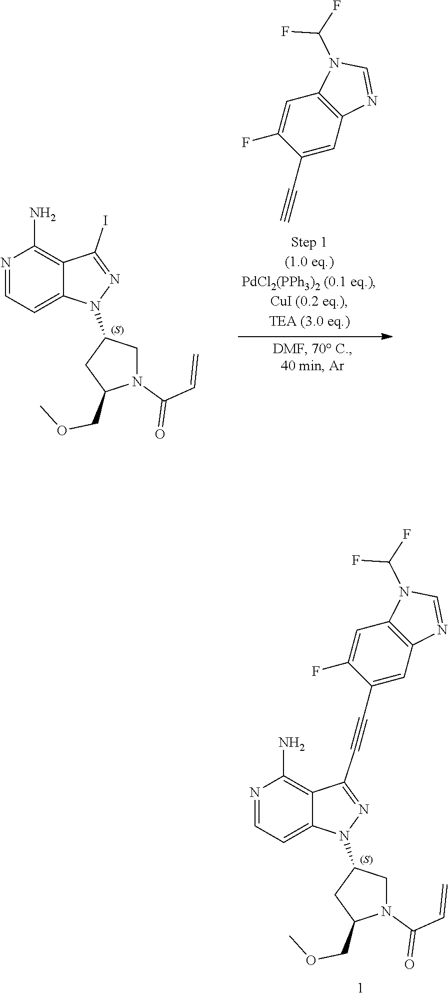

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (0.30 g, 0.70 mmol), 1-(difluoromethyl)-5-ethynyl-6-fluoro-1,3-benzodiazole (0.15 g, 0.70 mmol), Pd(PPh₃)₂Cl₂ (49.29 mg, 0.07 mmol) and CuI (26.75 mg, 0.14 mmol) in DMF (5.00 mL) was added TEA (0.21 g, 2.11 mmol) dropwise at room temperature. The reaction mixture was degassed with argon for three times and stirred for 40 min at 70° C. The resulting mixture was concentrated under reduced pressure. The residue was purified by silica gel column chromatography, eluted with DCM/MeOH (10/1) to afford the crude product. Then the crude product was purified by Prep-HPLC with the following conditions column: Sun-Fire Prep C18 OBD Column, 19×150 mm 5 μm 10 nm; Mobile Phase A: water (10 mmoL/L NH₄HCO₃), Mobile Phase B: ACN; Flow rate: 20 mL/min; Gradient: 20 B to 45 B in 30 min; 220/254 nm. The fractions contained desired product were combined and concentrated to afford 1-[(2R,4S)-4-(4-amino-3-[2-[1-(difluoromethyl)-6-fluoro-1,3-benzodiazol-5-yl]ethynyl]pyrazolo[4,3-c]pyridin-1-yl)-2-(methoxymethyl)pyrrolidin-1-yl]prop-2-en-1-one (0.24 g, 68%) as a white solid. MS ESI calculated for C₂₅H₂₂F₃N₇O₂ [M+H]⁺, 510.18, found 510.10; ¹H NMR (400 MHz, CDCl₃) δ 8.19 (s, 1H), 8.09 (dd, J=6.2, 4.0 Hz, 1H), 7.92-7.87 (m, 1H), 7.46-7.44 (m, 1H), 6.42 (d, J=63.86 Hz, 1H), 6.77-6.76 (m, 1H), 6.61-6.41 (m, 2H), 5.85-5.72 (m, 3H), 5.59-5.31 (m, 1H), 4.68-4.52 (m, 1H), 4.19-3.93 (m, 3H), 3.58-3.48 (m, 1H), 3.43 (s, 3H), 2.82-2.73 (m, 1H), 2.63-2.38 (m, 1H).

### Example 84: 1-[(2R,4S)-4-(4-Amino-5-[2-[1-(difluoromethyl)-6-fluoro-1,3-benzodiazol-5-yl]ethynyl]pyrrolo[2,3-d]pyrimidin-7-yl)-2-(methoxymethyl)pyrrolidin-1-yl]prop-2-en-1-one

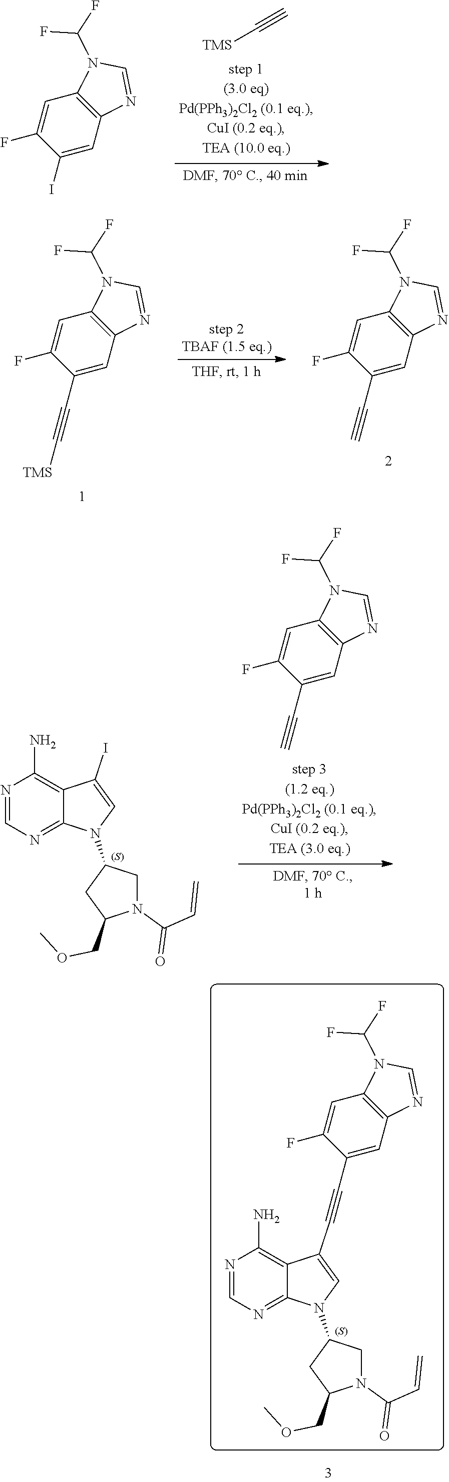

1-[(2R,4S)-4-(4-amino-5-[2-[1-(difluoromethyl)-6-fluoro-1,3-benzodiazol-5-yl] ethynyl]pyrrolo[2,3-d] pyrimidin-7-yl)-2-(methoxymethyl) pyrrolidin-1-yl] prop-2-en-1-one. MS ESI calculated for C₂₅H₂₂F₃N₇O₂ [M+H]⁺, 510.18, found 510.20; ¹H NMR (400 MHz, CDCl₃) δ 8.34 (s, 1H), 8.16 (s, 1H), 7.96 (d, J=6.2 Hz, 1H), 7.42 (d, J=8.7 Hz, 1H), 7.35-7.27 (m, 1H), 6.55-6.35 (m, 2H), 5.95-5.90 (m, 2H), 5.82-5.54 (m, 2H), 4.67 (dd, J=7.9, 4.0 Hz, 1H), 4.21 (dd, J=10.6, 7.4 Hz, 1H), 4.12-4.02 (m, 1H), 3.87-3.48 (m, 3H), 3.41 (s, 3H), 2.77-2.19 (m, 2H).

### Example 85: 1-[(2R,4S)-4-[4-Amino-3-[2-(1-ethyl-4,6-difluoro-1,3-benzodiazol-5-yl)ethynyl]pyrazolo[3,4-d]pyrimidin-1-yl]-2-(methoxymethyl)pyrrolidin-1-yl]prop-2-en-1-one

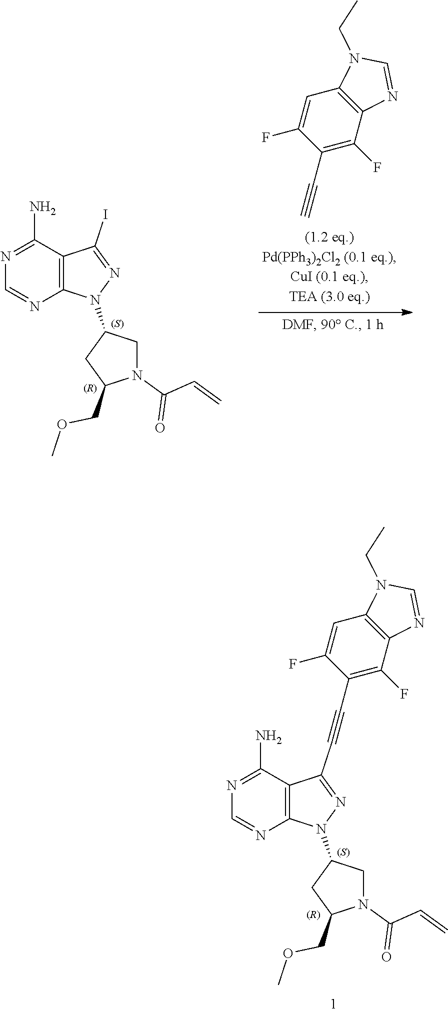

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.50 g, 1.17 mmol), 1-ethyl-5-ethynyl-4,6-difluoro-1,3-benzodiazole (0.29 g, 1.40 mmol), CuI (44.47 mg, 0.23 mmol) and Pd(PPh₃)₂Cl₂ (81.95 mg, 0.12 mmol) in DMF (5.00 mL) was added TEA (0.49 mL, 3.53 mmol) dropwise at room temperature. The reaction mixture was degassed with argon for three times and stirred for 1 h at 90° C. The resulting mixture was concentrated under reduced pressure. The residue was purified by silica gel column chromatography, eluted with CH₂Cl₂/MeOH (10/1) to afford crude product. The crude product was purified by Prep-HPLC with the following conditions column: C18 silica gel; mobile phase: MeCN in water (10 mmol/L NH₄HCO₃), 25% to 50% gradient in 40 min; detector: UV 254 nm. The fractions contained desired product were combined and concentrated to afford 1-[(2R,4S)-4-[4-amino-3-[2-(1-ethyl-4,6-difluoro-1,3-benzodiazol-5-yl)ethynyl]pyrazolo[3,4-d]pyrimidin-1-yl]-2-(methoxymethyl)pyrrolidin-1-yl]prop-2-en-1-one (0.19 g, 33%) as an off-white solid. MS ESI calculated for C₂₅H₂₄F₂N₈O₂ [M+H]⁺, 507.20, found 507.35; ¹H NMR (400 MHz, DMSO-d₆) δ 8.47 (s, 1H), 8.31-7.93 (m, 2H), 7.73 (d, J=8.4 Hz, 1H), 6.90-6.25 (m, 2H), 6.24-6.05 (m, 1H), 5.73-5.63 (m, 2H), 4.68-4.41 (m, 1H), 4.31 (q, J=7.2 Hz, 2H), 4.14-3.85 (m, 2H), 3.82-3.60 (m, 2H), 3.33-3.32 (s, 3H), 2.80-2.53 (m, 1H), 2.42-2.39 (m, 1H), 1.42 (t, J=7.2 Hz, 3H).

### Example 86: 1-[(2R,4S)-4-[4-Amino-3-[2-(1-ethyl-4,6-difluoro-1,3-benzodiazol-5-yl)ethynyl]pyrazolo[4,3-c]pyridin-1-yl]-2-(methoxymethyl) pyrrolidin-1-yl]prop-2-en-1-one

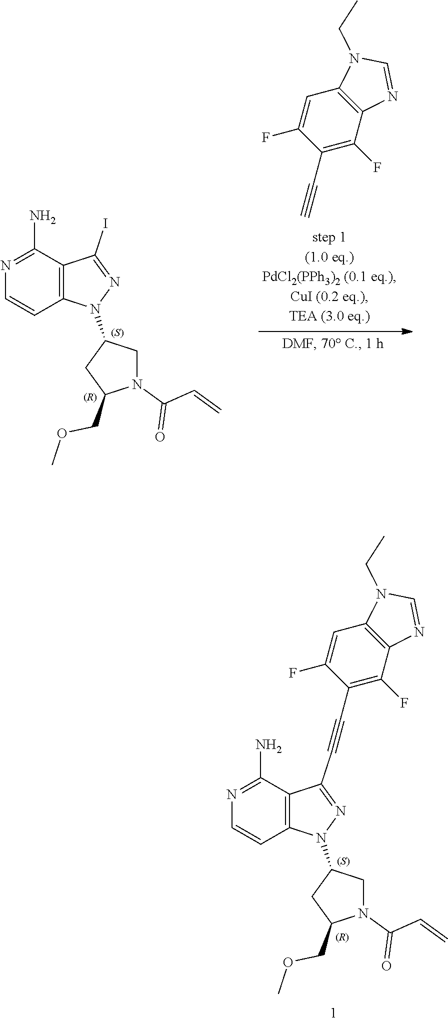

To a stirred mixture of 1-((2R,4S)-4-(4-amino-3-iodo-1H-pyrazolo[4,3-c]pyridin-1-yl)-2-(methoxymethyl)pyrrolidin-1-yl)prop-2-en-1-one (0.45 g, 1.05 mmol) and 1-ethyl-5-ethynyl-4,6-difluoro-1,3-benzodiazole (0.22 g, 1.05 mmol) in DMF (80.00 mL) were added Pd(PPh₃)₂Cl₂ (73.93 mg, 0.11 mmol), CuI (40.12 mg, 0.21 mmol) and TEA (0.32 g, 3.16 mmol) at room temperature. The reaction mixture was degassed with argon for three times and stirred for 1 h at 70° C. The resulting mixture was concentrated under reduced pressure. The residue was purified by silica gel column chromatography, eluted with CH₂Cl₂/MeOH (10/1). The fractions contained desired product were combined and concentrated. The residue was purified by reverse flash chromatography with the following conditions: column: C18 silica gel; mobile phase: ACN in water (10 mmol/L NH₄HCO₃), 5% to 70% gradient in 30 min; detector: UV 254 nm. The fractions contained desired product were combined and concentrated to afford 1-[(2R,4S)-4-[4-amino-3-[2-(1-ethyl-4,6-difluoro-1,3-benzodiazol-5-yl)ethynyl]pyrazolo[4,3-c]pyridin-1-yl]-2-(methoxymethyl)pyrrolidin-1-yl]prop-2-en-1-one (0.35 g, 65%) as a light yellow solid. MS ESI calculated for C₂₆H₂₅F₂N₇O₂ [M+H]⁺, 506.21, found 506.10; ¹H NMR (400 MHz, DMSO-d₆) δ 8.47 (d, J=1.8 Hz, 1H), 7.86-7.71 (m, 2H), 7.00 (dd, J=6.7, 2.5 Hz, 1H), 6.81-6.54 (m, 3H), 6.17 (m, 1H), 5.69 (m, 1H), 5.52 (t, J=6.7 Hz, 1H), 4.66-4.44 (m, 1H), 4.31 (m, 2H), 4.13-3.74 (m, 2H), 3.65-3.44 (m, 2H), 3.32 (s, 3H), 2.77-2.52 (m, 1H), 2.46-2.35 (m, 1H), 1.42 (t, J=7.3 Hz, 3H).

### Example 87: 1-[(2R,4S)-4-[4-Amino-5-[2-(1-ethyl-4,6-difluoro-1,3-benzodiazol-5-yl)ethynyl]pyrrolo[2,3-d]pyrimidin-7-yl]-2-(methoxymethyl)pyrrolidin-1-yl]prop-2-en-1-one

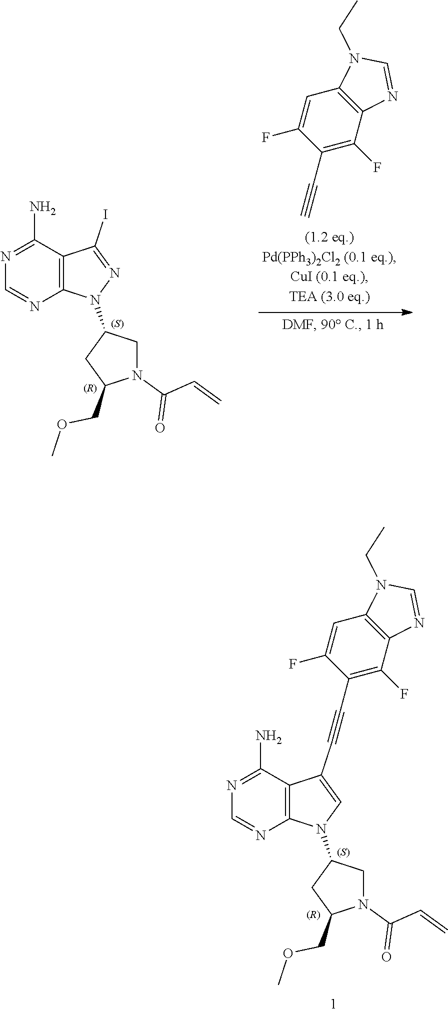

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (0.50 g, 1.17 mmol), 1-ethyl-5-ethynyl-4,6-difluoro-1,3-benzodiazole (0.29 g, 1.40 mmol), CuI (44.58 mg, 0.23 mmol) and Pd(PPh₃)₂Cl₂ (82.14 mg, 0.12 mmol) in DMF (5.00 mL) was added TEA (0.50 mL, 3.60 mmol) dropwise at room temperature. The reaction mixture was degassed with argon for three times and stirred for 1 h at 90° C. The resulting mixture was concentrated under reduced pressure. The residue was purified by silica gel column chromatography, eluted with CH₂Cl₂/MeOH (10/1) to afford crude product. The crude product was purified by Prep-HPLC with the following conditions column: C18 silica gel; mobile phase: MeCN in water (10 mmol/L NH₄HCO₃), 25% to 50% gradient in 40 min; detector: UV 254 nm. The fractions contained desired product were combined and concentrated to afford 1-[(2R,4S)-4-[4-amino-5-[2-(1-ethyl-4,6-difluoro-1,3-benzodiazol-5-yl)ethynyl]pyrrolo[2,3-d]pyrimidin-7-yl]-2-(methoxymethyl)pyrrolidin-1-yl]prop-2-en-1-one (0.39 g, 66%) as an off-white solid. MS ESI calculated for C₂₆H₂₅F₂N₇O₂ [M+H]⁺, 506.20, found 506.35; ¹H NMR (400 MHz, DMSO-d₆) δ 8.43 (s, 1H), 8.20 (d, J=4.0 Hz, 1H), 7.90 (d, J=20.8 Hz, 1H), 7.68 (d, J=9.2 Hz, 1H), 6.82-6.71 (m, 1H), 6.62-6.54 (m, 1H), 6.21-6.15 (m, 1H), 5.71-5.67 (m, 1H), 5.60-5.39 (m, 1H), 4.67-4.41 (m, 1H), 4.36-4.30 (m, 2H), 4.16-3.78 (m, 2H), 3.65-3.42 (m, 2H), 3.32 (s, 3H), 2.76-2.54 (m, 1H), 2.43-2.31 (m, 1H), 1.41 (t, J=7.2 Hz, 3H).

### Example 88: 1-[(2R,4S)-4-[4-Amino-3-[2-(6-chloro-1-ethyl-1,3-benzodiazol-5-yl)ethynyl]pyrazolo[4,3-c]pyridin-1-yl]-2-(methoxymethyl)pyrrolidin-1-yl]prop-2-en-1-one

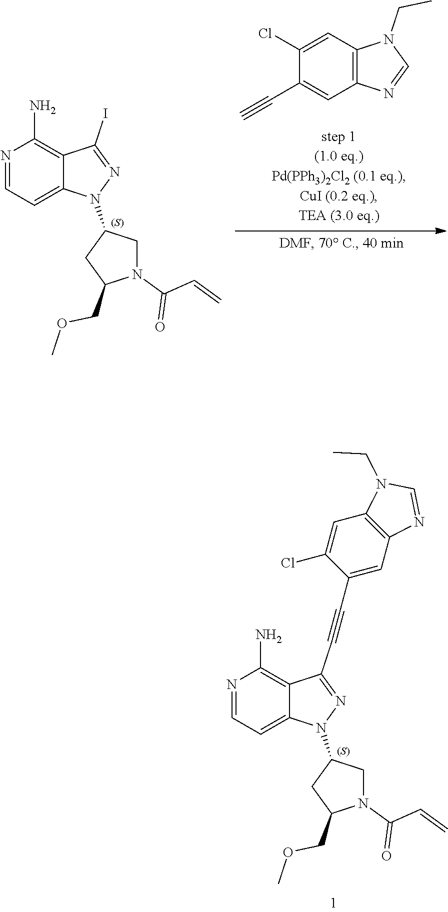

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (0.45 g, 1.05 mmol) and 6-chloro-1-ethyl-5-ethynyl-1,3-benzodiazole (0.22 g, 1.05 mmol) in DMF (5.00 mL) were added CuI (40.12 mg, 0.21 mmol), Pd(PPh₃)₂Cl₂ (73.93 mg, 0.11 mmol) and TEA (0.32 g, 3.16 mmol) at room temperature. The reaction mixture was degassed with argon for three times and stirred for 40 min at 70° C. The resulting mixture was concentrated under reduced pressure. The residue was purified by silica gel column chromatography, eluted with CH₂Cl₂/MeOH (10/1) to afford the crude product. The crude product was purified by Prep-HPLC with the following conditions: column, SunFire Prep C18 OBD Column, 19×150 mm 5 μm 10 nm; Mobile Phase A: water (10 mmol/L NH₄HCO₃); Mobile Phase B: ACN; Flow rate: 50 mL/min; 5%-15% within 40 min; Detector: UV 254/210 nm. The fractions contained desired product were combined and concentrated to afford 1-[(2R,4S)-4-[4-amino-3-[2-(6-chloro-1-ethyl-1,3-benzodiazol-5-yl)ethynyl]pyrazolo[4,3-c]pyridin-1-yl]-2-(methoxymethyl)pyrrolidin-1-yl]prop-2-en-1-one (0.24 g, 45%) as a white solid. MS ESI calculated for C₂₆H₂₆ClN₇O₂ [M+H]⁺, 504.18, found 504.05; ¹H NMR (400 MHz, CDCl₃) δ 8.09-8.02 (m, 3H), 7.58 (s, 1H), 6.76 (s, 1H), 6.45 (d, J=7.2 Hz, 2H), 5.80-5.71 (m, 3H), 5.56-5.50 (m, 1H), 4.66 (d, J=8.9 Hz, 1H), 4.25 (d, J=7.4 Hz, 2H), 4.16-4.12 (m, 2H), 3.95-3.91 (m, 1H), 3.53-3.49 (m, 1H), 3.43 (s, 3H), 2.82-2.74 (m, 1H), 2.47-2.42 (m, 1H), 1.62-1.58 (m, 3H).

### Example 89: 1-[(2R,4S)-4-[4-Amino-5-[2-(6-chloro-2-methyl-1H-1,3-benzodiazol-5-yl)ethynyl]pyrrolo[2,3-d]pyrimidin-7-yl]-2-(methoxymethyl)pyrrolidin-1-yl]prop-2-en-1-one

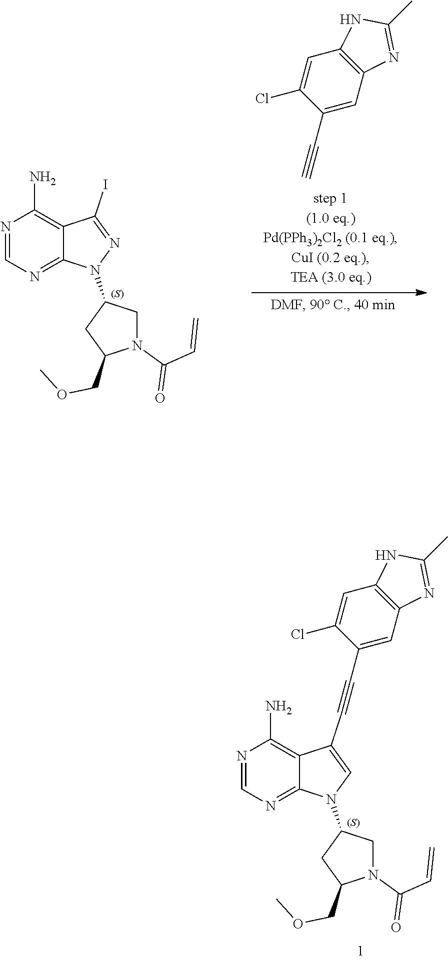

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (0.45 g, 1.05 mmol), 5-chloro-6-ethynyl-2-methyl-3H-1,3-benzodiazole (0.20 g, 1.05 mmol), Pd(PPh₃)₂Cl₂ (73.93 mg, 0.11 mmol) and CuI (40.12 mg, 0.21 mmol) in DMF (4.50 mL) was added TEA (0.44 mL, 3.16 mmol). The reaction mixture was degassed with argon for three times and stirred for 40 min at 90° C. The resulting mixture was concentrated under reduced pressure. The residue was purified by silica gel column chromatography, eluted with DCM/MeOH (10/1). The fractions contained desired product were combined and concentrated. The residue was purified by reverse flash chromatography with the following conditions: Column: SunFire Prep C18 OBD Column, Mobile Phase A: water (10 mmoL NH₄HCO₃), Mobile Phase B: ACN; Flow rate: 80 mL/min; Gradient: 10 B to 40 B in 30 min; 210/254 nm. The fractions contained desired product were combined and concentrated to afford 1-[(2R,4S)-4-[4-amino-5-[2-(6-chloro-2-methyl-1H-1,3-benzodiazol-5-yl)ethynyl]pyrrolo[2,3-d]pyrimidin-7-yl]-2-(methoxymethyl)pyrrolidin-1-yl]prop-2-en-1-one (0.23 g, 44%) as an off-white solid. MS ESI calculated for C₂₅H₂₄ClN₇O₂ [M+H]⁺, 490.17, found 490.10; ¹H NMR (400 MHz, CDCl₃) δ 8.32 (s, 1H), 7.66 (d, J=35.2 Hz, 2H), 7.26 (d, J=3.9 Hz, 1H), 6.70-6.33 (m, 2H), 6.02 (brs, 2H), 5.85-5.67 (m, 1H), 5.59-5.51 (m, 1H), 4.71-4.52 (m, 1H), 4.19-4.07 (m, 2H), 3.88-3.74 (m, 1H), 3.61-3.47 (m, 1H), 3.40 (d, J=3.6 Hz, 3H), 2.73-2.40 (m, 5H).

### Example 90: 1-[(2R,4S)-4-[4-Amino-3-[2-(6-chloro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one

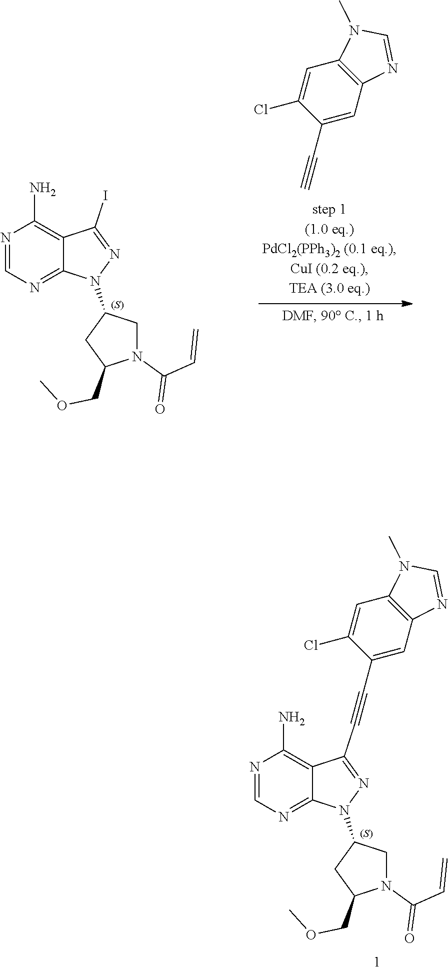

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.55 g, 1.28 mmol) and 6-chloro-5-ethynyl-1-methyl-1,3-benzodiazole (0.25 g, 1.28 mmol) in DMF (9.00 mL) were added Pd(PPh₃)₂Cl₂ (90.15 mg, 0.13 mmol), CuI (48.92 mg, 0.26 mmol) and TEA (0.53 mL, 3.84 mmol) at room temperature. The reaction mixture was degassed with argon for three times and stirred for 1 h at 90° C. The resulting mixture was concentrated under reduced pressure. The residue was purified by silica gel column chromatography, eluted with CH₂Cl₂/MeOH (10/1). The fractions contained desired product were combined and concentrated. The crude product was further purified by Prep-HPLC with the following conditions Column: XBridge Prep C18 OBD Column, 19×150 mm 5 μm; Mobile Phase A: water (10 mmol/L NH₄HCO₃), Mobile Phase B: ACN; Flow rate: 20 mL/min; Gradient: 35 B to 60 B in 5.8 min; 210/254 nm; RT1: 5.85 min. The fractions contained desired product were combined and concentrated to afford 1-[(2R,4S)-4-[4-amino-3-[2-(6-chloro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one (0.18 g, 29%) as an off-white solid. MS ESI calculated for C₂₄H₂₃ClN₈O₂ [M+H]⁺, 491.16, found 491.05; ¹H NMR (400 MHz, DMSO-d₆) δ 8.36 (s, 1H), 8.29 (d, J=2.6 Hz, 1H), 8.20 (s, 1H), 7.96 (s, 1H), 6.67-6.54 (m, 2H), 6.27-6.17 (m, 1H), 5.76-5.57 (m, 2H), 4.67-4.54 (m, 1H), 4.14-3.78 (m, 5H), 3.66-3.46 (m, 2H), 3.32 (s, 3H), 2.75-2.53 (m, 1H), 2.48-2.39 (m, 1H).

### Example 91: 1-[(2R,4S)-4-[4-Amino-5-[2-(6-chloro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one

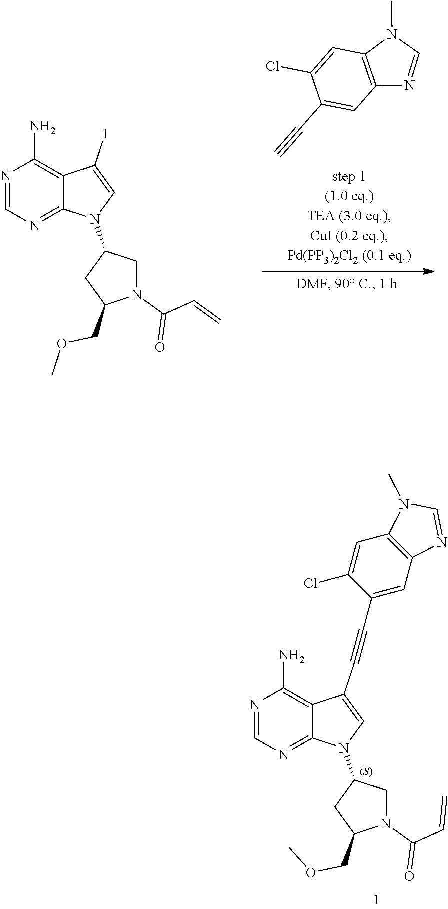

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (0.45 g, 1.05 mmol), 6-chloro-5-ethynyl-1-methyl-1,3-benzodiazole (0.20 g, 1.05 mmol), Pd(PPh₃)₂Cl₂ (73.93 mg, 0.11 mmol) and CuI (40.12 mg, 0.21 mmol) in DMF (4.50 mL) was added TEA (0.44 mL, 3.16 mmol) at room temperature. The reaction mixture was degassed with argon for three times and stirred for 1 h at 90° C. The resulting mixture was concentrated under reduced pressure. The residue was purified by silica gel column chromatography, eluted with CH₂Cl₂/MeOH (10/1) to afford crude product which was further purified by Prep-HPLC with the following conditions Column: XBridge Prep C18 OBD Column, 19×150 mm 5 μm; Mobile Phase A: water (10 mmol/L NH₄HCO₃), Mobile Phase B: ACN; Flow rate: 20 mL/min; Gradient: 35 B to 50 B in 5.8 min; 210/254 nm; RT1:5.58 min. The fractions contained desired product were combined and concentrated to afford 1-[(2R,4S)-4-[4-amino-5-[2-(6-chloro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one (0.26 g, 56%) as a white solid. MS ESI calculated for C₂₅H₂₄ClN₇O₂ [M+H]⁺, 490.17, found 490.96; ¹H NMR (300 MHz, CDCl₃) δ 8.32 (s, 1H), 8.00-7.80 (m, 2H), 7.52 (s, 1H), 7.29 (d, J=4.9 Hz, 1H), 6.63-6.36 (m, 2H), 5.90 (s, 2H), 5.81-5.54 (m, 2H), 4.53 (d, J=73.8 Hz, 1H), 4.27-3.94 (m, 1H), 3.88-3.74 (m, 5H), 3.58-3.55 (m, 1H), 3.39 (s, 3H), 2.58-2.52 (m, 2H).

### Example 92: 1-[(2R,4S)-4-[4-Amino-3-[2-(6-chloro-1-ethyl-1,3-benzodiazol-5-yl)ethynyl]pyrazolo[3,4-d]pyrimidin-1-yl]-2-(methoxymethyl)pyrrolidin-1-yl]prop-2-en-1-one

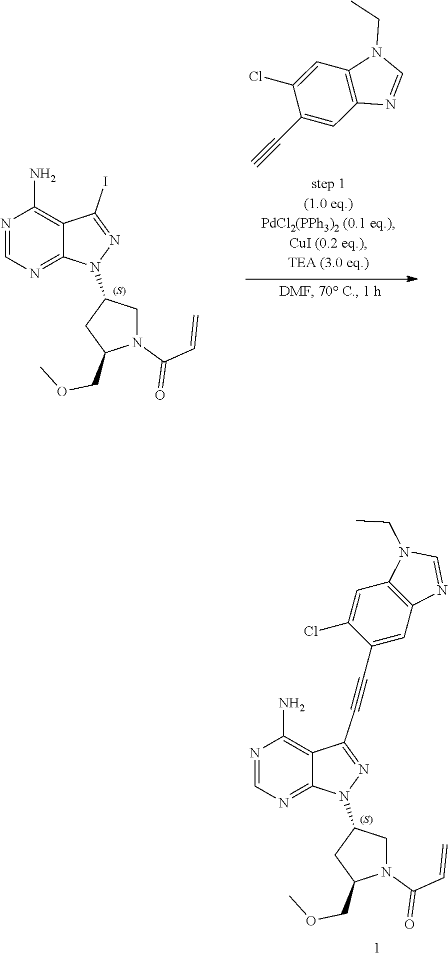

To a stirred mixture of 1-[(2R,4S)-4-[4-amino-3-iodopyrazolo[3,4-d] pyrimidin-1-yl]-2-(methoxymethyl) pyrrolidin-1-yl]prop-2-en-1-one (0.45 g, 1.05 mmol) and 6-chloro-1-ethyl-5-ethynyl-1,3-benzodiazole (0.22 mg, 1.05 mmol) in DMF (11.00 mL) were added Pd(PPh₃)₂Cl₂ (73.76 mg, 0.11 mmol), CuI (40.03 mg, 0.21 mmol) and TEA (0.32 g, 3.15 mmol) at room temperature. The reaction mixture was degassed with argon for three times and stirred for 1 h at 70° C. The resulting mixture was concentrated under reduced pressure. The residue was purified by silica gel column chromatography, eluted with CH₂Cl₂/MeOH (10/1). The fractions contained desired product were combined and concentrated. The residue was purified by reverse flash chromatography with the following conditions: column, C18 silica gel; mobile phase; ACN in water (10 mmol/L NH₄HCO₃), 5% to 70% gradient in 30 min; detector: UV 254 nm. The fractions contained desired product were combined and concentrated to afford 1-[(2R,4S)-4-[4-amino-3-[2-(6-chloro-1-ethyl-1,3-benzodiazol-5-yl)ethynyl]pyrazolo[3,4-d]pyrimidin-1-yl]-2-(methoxymethyl)pyrrolidin-1-yl]prop-2-en-1-one (0.24 g, 43%) as an off-white solid. MS ESI calculated for C₂₅H₂₅ClN₈O₂ [M+H]⁺, 505.19, found 505.20; ¹H NMR (400 MHz, DMSO-d₆) δ 8.47 (s, 1H), 8.32-8.16 (m, 2H), 8.04 (s, 1H), 6.81-6.58 (m, 1H), 6.58-6.20 (m, 1H), 6.20-6.13 (m, 1H), 5.69 (m, 2H), 4.55 (d, J=57.1 Hz, 1H), 4.37-4.31 (m, 2H), 3.96 (m, 2H), 3.65-3.62 (m, 1H), 3.56-3.46 (m, 2H), 3.33 (d, J=5.2 Hz, 3H), 2.76-2.54 (m, 1H), 2.43-2.34 (m, 1H), 1.42 (t, J=7.2 Hz, 3H).

### Example 93: 1-[(2R,4S)-4-[4-Amino-5-[2-(6-chloro-1-ethyl-1,3-benzodiazol-5-yl)ethynyl]pyrrolo[2,3-d]pyrimidin-7-yl]-2-(methoxymethyl)pyrrolidin-1-yl]prop-2-en-1-one

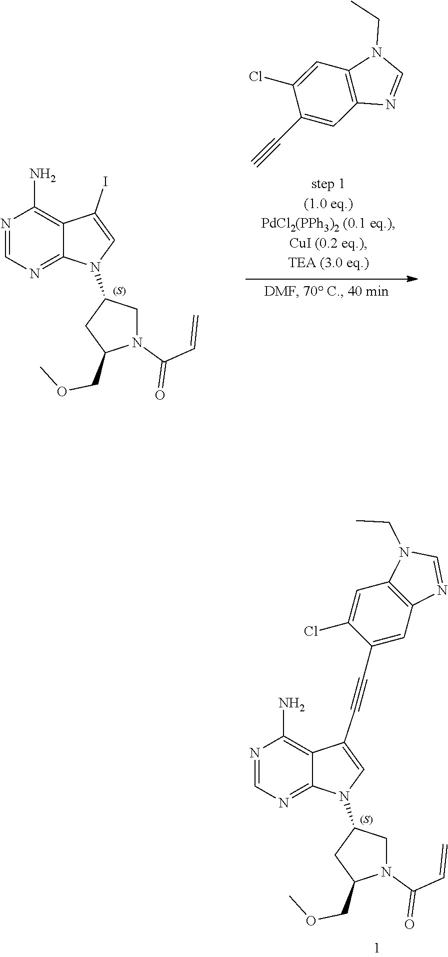

To a stirred mixture of 1-[(2R,4S)-4-[4-amino-5-iodopyrrolo[2,3-d]pyrimidin-7-yl]-2-(methoxymethyl)pyrrolidin-1-yl]prop-2-en-1-one (0.50 g, 1.17 mmol) and 6-chloro-1-ethyl-5-ethynyl-1,3-benzodiazole (0.24 g, 1.17 mmol) in DMF (12.00 mL) were added Pd(PPh₃)₂Cl₂ (82.14 mg, 0.12 mmol), CuI (44.58 mg, 0.23 mmol) and TEA (0.36 g, 3.51 mmol) at room temperature. The reaction mixture was degassed with argon for three times and stirred for 40 min at 70° C. The resulting mixture was concentrated under reduced pressure. The residue was purified by silica gel column chromatography, eluted with CH₂Cl₂/MeOH (10/1). The fractions contained desired product were combined and concentrated. The residue was purified by reverse flash chromatography with the following conditions: column: C18 silica gel; mobile phase: ACN in water (10 mmol/L NH₄HCO₃), 5% to 70% gradient in 30 min; detector: UV 254 nm. The fractions contained desired product were combined and concentrated to afford 1-[(2R,4S)-4-[4-amino-5-[2-(6-chloro-1-ethyl-1,3-benzodiazol-5-yl)ethynyl]pyrrolo[2,3-d]pyrimidin-7-yl]-2-(methoxymethyl)pyrrolidin-1-yl]prop-2-en-1-one (0.23 g, 39%) as an off-white solid. MS ESI calculated for C₂₆H₂₆ClN₇O₂ [M+H]⁺, 504.19, found 504.25; ¹H NMR (400 MHz, CDCl₃) δ 8.32 (s, 1H), 8.02 (s, 2H), 7.54 (s, 1H), 7.31-7.29 (m, 1H), 6.65-6.31 (m, 2H), 5.93 (s, 2H), 5.77-5.53 (m, 2H), 4.68-4.36 (m, 1H), 4.26-4.00 (m, 3H), 3.87-3.76 (m, 1H), 3.60-3.45 (m, 1H), 3.39 (s, 3H), 2.66-2.45 (m, 2H), 1.89-1.81 (m, 1H), 1.62-1.48 (m, 3H).

### Example 94: 1-[(2R,4S)-4-[4-Amino-3-[2-(4,6-difluoro-2-methyl-1H-1,3-benzodiazol-5-yl)ethynyl]pyrazolo[3,4-d]pyrimidin-1-yl]-2-(methoxymethyl)pyrrolidin-1-yl]prop-2-en-1-one

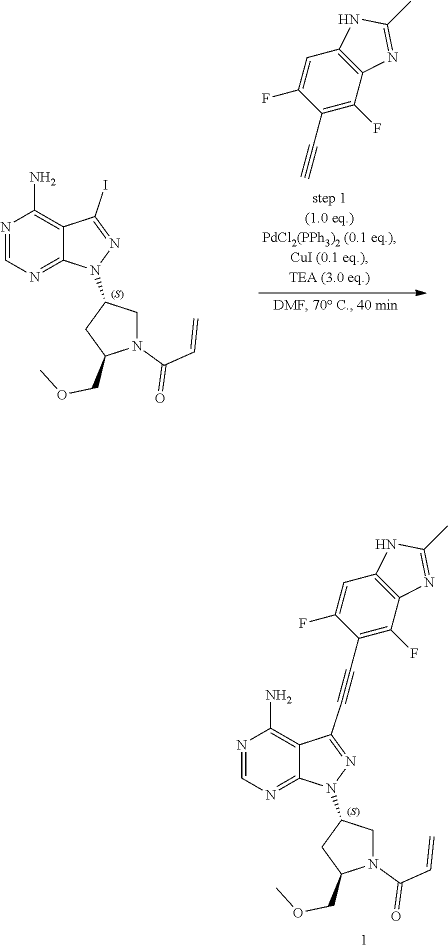

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.47 g, 1.10 mmol), 5-ethynyl-4,6-difluoro-2-methyl-1H-1,3-benzodiazole (0.21 g, 1.10 mmol), Pd(PPh₃)₂Cl₂ (77.04 mg, 0.11 mmol) and CuI (20.90 mg, 0.11 mmol) in DMF (8.00 mL) was added TEA (0.33 g, 3.29 mmol) dropwise at room temperature. The reaction mixture was degassed with argon for three times and stirred for 40 min at 70° C. The resulting mixture was concentrated under reduced pressure. The residue was purified by silica gel column chromatography, eluted with DCM/MeOH (10/1) to afford crude product. Then the crude product was purified by Prep-HPLC with the following conditions: Column: XBridge Prep C18 OBD Column, 19×150 mm 5 μm; Mobile Phase A: water (10 mmol/L NH₄HCO₃), Mobile Phase B: ACN; Flow rate: 20 mL/min; Gradient: 35 B to 55 B in 5.8 min; 210/254 nm; RT1: 5.58 min. The fractions contained desired product were combined and concentrated to afford 1-[(2R,4S)-4-[4-amino-3-[2-(4,6-difluoro-2-methyl-1H-1,3-benzodiazol-5-yl)ethynyl]pyrazolo[3,4-d]pyrimidin-1-yl]-2-(methoxymethyl)pyrrolidin-1-yl]prop-2-en-1-one (0.15 g, 27%) as an off-white solid. MS ESI calculated for C₂₄H₂₂F₂N₈O₂[M+H]⁺, 493.18, found 493.05; ¹H NMR (400 MHz, CDCl₃) δ 8.33 (d, J=9.6 Hz, 1H), 7.07-7.02 (m, 1H), 6.55-6.28 (m, 3H), 5.91-5.56 (m, 2H), 4.75-4.55 (m, 1H), 4.44-3.77 (m, 3H), 3.67-3.33 (m, 4H), 3.05-2.74 (m, 1H), 2.64 (d, J=13.6 Hz, 3H), 2.60-2.41 (m, 1H).

### Example 95: 1-[(2R,4S)-4-[4-Amino-3-[2-(4,6-difluoro-2-methyl-1H-1,3-benzodiazol-5-yl)ethynyl]pyrazolo[4,3-c]pyridin-1-yl]-2-(methoxymethyl)pyrrolidin-1-yl]prop-2-en-1-one

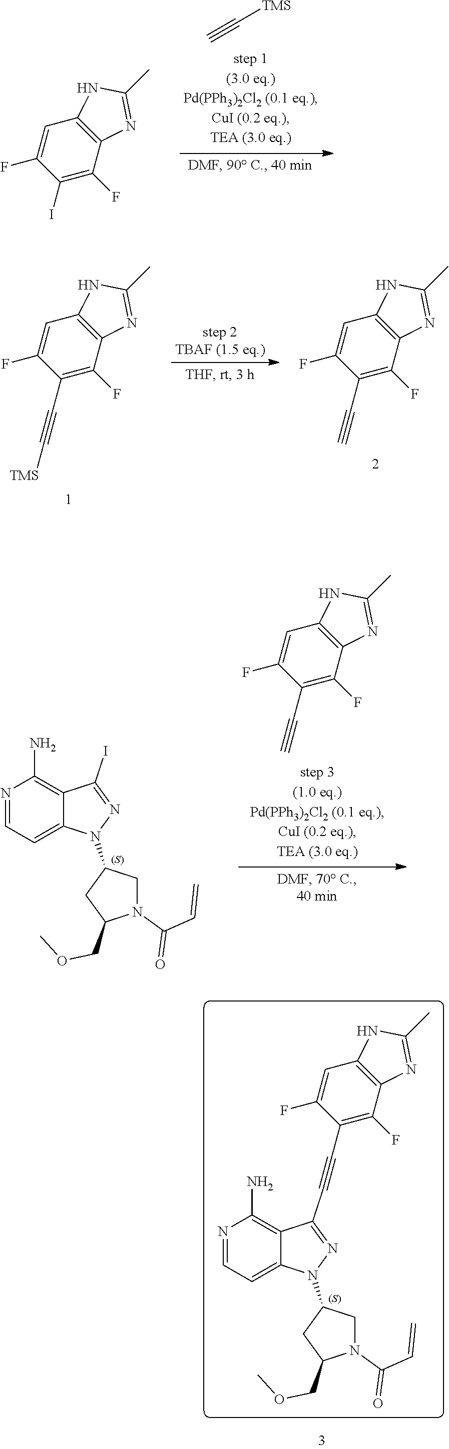

1-[(2R,4S)-4-[4-amino-3-[2-(4,6-difluoro-2-methyl-1H-1,3-benzodiazol-5-yl)ethynyl]pyrazolo[4,3-c]pyridin-1-yl]-2-(methoxymethyl)pyrrolidin-1-yl]prop-2-en-1-one. MS ESI calculated for C₂₅H₂₃F₂N₇O₂ [M+H]⁺, 492.19, found 492.10; ¹H NMR (400 MHz, CDCl₃) δ 7.83-7.80 (m, 1H), 7.12-7.04 (m, 1H), 6.74 (d, J=6.5 Hz, 1H), 6.68-6.34 (m, 2H), 6.19-6.12 (m, 2H), 5.84-5.63 (m, 1H), 5.54-5.17 (m, 1H), 4.76-4.46 (m, 1H), 4.28-3.85 (m, 3H), 3.61-3.32 (m, 4H), 2.83-2.34 (m, 5H).

### Example 96: 1-[(2R,4S)-4-[4-Amino-3-[2-(6-chloro-1,2-dimethyl-1,3-benzodiazol-5-yl)ethynyl]pyrazolo[3,4-d]pyrimidin-1-yl]-2-(methoxymethyl)pyrrolidin-1-yl]prop-2-en-1-one

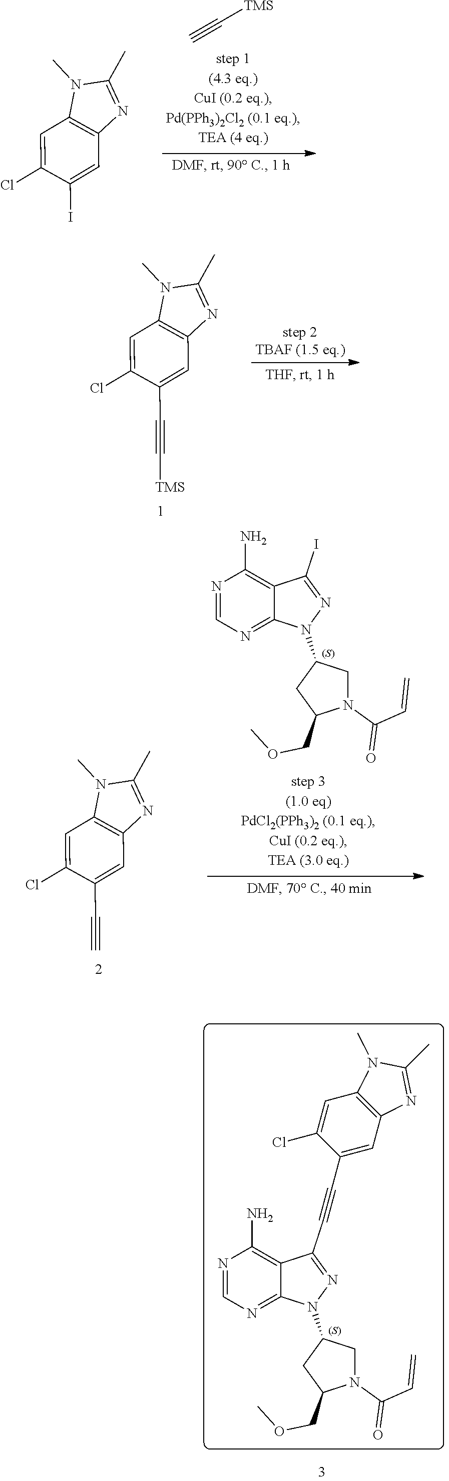

1-[(2R,4S)-4-[4-amino-3-[2-(6-chloro-1,2-dimethyl-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₅H₂₅ClN₈O₂ [M+H]⁺, 505.14, found 505.15; ¹H NMR (400 MHz, CDCl₃) δ 8.40 (d, J=5.6 Hz, 1H), 8.00 (d, J=5.8 Hz, 1H), 7.42 (s, 1H), 6.66-6.39 (m, 2H), 6.20 (s, 1H), 5.95-5.63 (m, 2H), 4.75-4.49 (m, 1H), 4.26-3.97 (m, 2H), 3.88-3.82 (m, 1H), 3.76 (s, 3H), 3.58-3.49 (m, 1H), 3.42 (s, 3H), 3.04-2.77 (m, 1H), 2.65 (s, 3H), 2.52-2.47 (m, 1H).

### Example 97: 1-[(2R,4S)-4-[4-Amino-3-[2-(6-chloro-1,2-dimethyl-1,3-benzodiazol-5-yl)ethynyl]pyrazolo[4,3-c]pyridin-1-yl]-2-(methoxymethyl)pyrrolidin-1-yl]prop-2-en-1-one

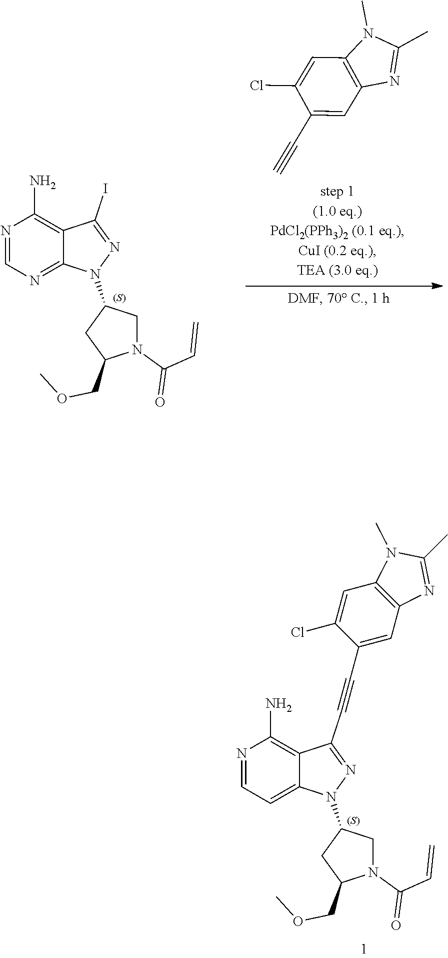

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (0.45 g, 1.05 mmol) and 6-chloro-5-ethynyl-1,2-dimethyl-1,3-benzodiazole (0.22 g, 1.05 mmol) in DMF (11.00 mL) were added Pd(PPh₃)₂Cl₂ (73.93 mg, 0.11 mmol), CuI (40.12 mg, 0.21 mmol) and TEA (0.32 g, 3.16 mmol) at room temperature. The reaction mixture was degassed with argon for three times and stirred for 1 h at 70° C. The resulting mixture was concentrated under reduced pressure. The residue was purified by silica gel column chromatography, eluted with CH₂Cl₂/MeOH (10/1). The fractions contained desired product were combined and concentrated. The residue was purified by reverse flash chromatography with the following conditions: column: C18 silica gel; mobile phase: ACN in water (10 mmol/L NH₄HCO₃), 10% to 50% gradient in 30 min; detector; UV 254 nm. The fractions contained desired product were combined and concentrated to afford 1-[(2R,4S)-4-[4-amino-3-[2-(6-chloro-1,2-dimethyl-1,3-benzodiazol-5-yl)ethynyl]pyrazolo[4,3-c]pyridin-1-yl]-2-(methoxymethyl)pyrrolidin-1-yl]prop-2-en-1-one (0.16 g, 30%) as an off-white solid. MS ESI calculated for C₂₆H₂₆ClN₇O₂ [M+H]⁺, 504.19, found 504.10; ¹H NMR (400 MHz, CDCl₃) δ 7.99 (d, J=5.2 Hz, 1H), 7.90 (d, J=6.5 Hz, 1H), 7.40 (d, J=2.2 Hz, 1H), 6.72 (t, J=7.3 Hz, 1H), 6.51-6.38 (m, 2H), 5.83-5.66 (m, 3H), 5.53 (m, 1H), 4.66 (d, J=8.9 Hz, 1H), 4.20-4.07 (m, 2H), 3.94-3.91 (m, 1H), 3.75 (s, 3H), 3.58-3.47 (m, 1H), 3.43 (s, 3H), 2.78 (m, 1H), 2.64 (s, 3H), 2.57-2.40 (m, 1H).

### Example 98: 1-[(2R,4S)-4-[4-Amino-3-[2-(6-chloro-1-methyl-1,3-benzodiazol-5-yl)ethynyl]pyrazolo[4,3-c]pyridin-1-yl]-2-(methoxymethyl)pyrrolidin-1-yl]prop-2-en-1-one

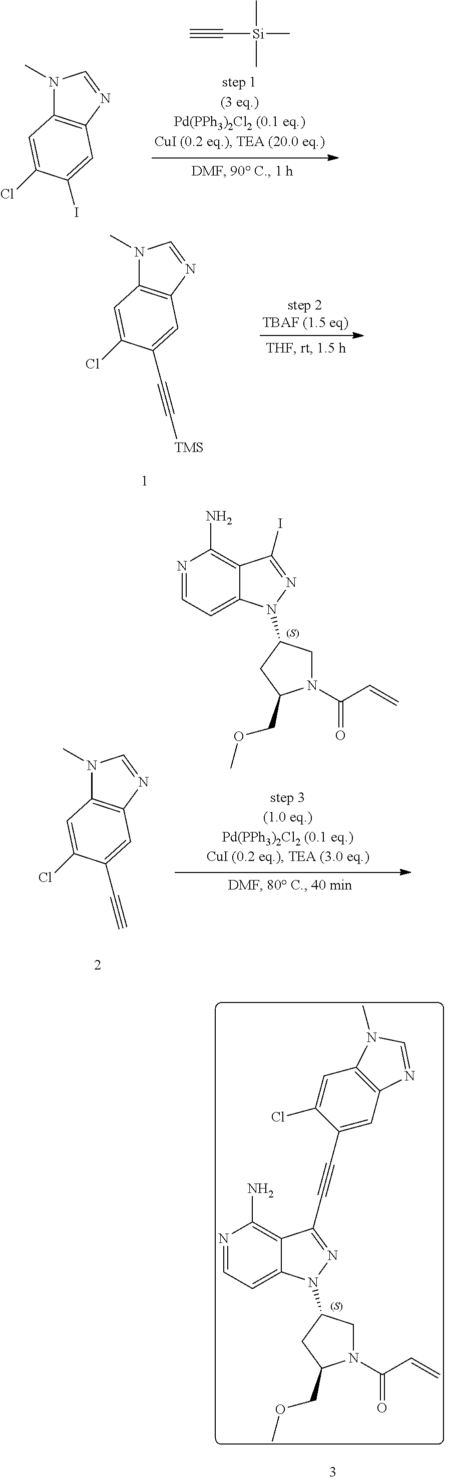

1-[(2R,4S)-4-[4-amino-3-[2-(6-chloro-1-methyl-1,3-benzodiazol-5-yl)ethynyl]pyrazolo[4,3-c]pyridin-1-yl]-2-(methoxymethyl)pyrrolidin-1-yl]prop-2-en-1-one. MS ESI calculated for C₂₅H₂₄ClN₇O₂ [M+H]⁺, 490.17, found 490.05; ¹H NMR (400 MHz, CDCl₃) δ 8.14 (d, J=5.2 Hz, 1H), 7.95 (s, 1H), 7.91 (d, J=6.4 Hz, 1H), 7.53 (s, 1H), 6.77-6.69 (m, 1H), 6.52-6.38 (m, 2H), 5.86-5.66 (m, 3H), 5.53-5.50 (m, 1H), 4.66-4.64 (m, 1H), 4.20-4.00 (m, 2H), 3.93-3.89 (m, 1H), 3.88 (s, 3H), 3.57-3.47 (m, 1H), 3.43 (s, 3H), 2.78-2.73 (m, 1H), 2.49-2.45 (m, 1H).

### Example 99: 1-[(2R,4S)-4-[4-Amino-3-[2-(6-chloro-2-methyl-1H-1,3-benzodiazol-5-yl)ethynyl]pyrazolo[3,4-d]pyrimidin-1-yl]-2-(methoxymethyl)pyrrolidin-1-yl]prop-2-en-1-one

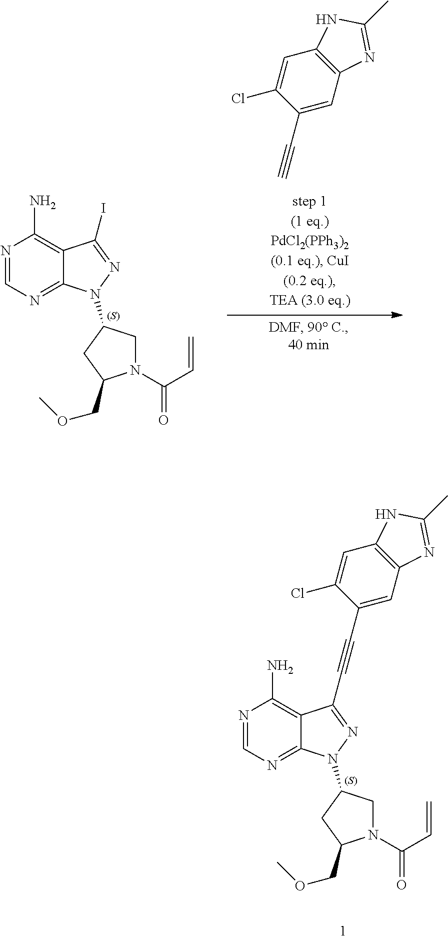

To a stirred mixture of 1-[(2R,4S)-4-[4-amino-3-iodopyrazolo[3,4-d]pyrimidin-1-yl]-2-(methoxymethyl)pyrrolidin-1-yl]prop-2-en-1-one (0.40 g, 0.93 mmol), 5-chloro-6-ethynyl-2-methyl-3H-1,3-benzodiazole (0.18 g, 0.93 mmol), Pd(PPh₃)₂Cl₂ (65.56 mg, 0.09 mmol) and CuI (35.58 mg, 0.18 mmol) in DMF (1.00 mL) was added TEA (0.28 g, 2.80 mmol) dropwise at room temperature. The reaction mixture was degassed with argon for three times and stirred for 40 min at 90° C. The resulting mixture was concentrated under reduced pressure. The residue was purified by silica gel column chromatography, eluted with CH₂Cl₂/MeOH (10/1). The fractions contained desired product were combined and concentrated. The residue was purified by reverse flash chromatography with the following conditions: column: C18 silica gel; mobile phase: ACN in water (10 mmol/L NH₄HCO₃), 10% to 40% gradient in 30 min; detector: UV 254 nm. The fractions contained desired product were combined and concentrated to afford 1-[(2R,4S)-4-[4-amino-3-[2-(6-chloro-2-methyl-1H-1,3-benzodiazol-5-yl)ethynyl]pyrazolo[3,4-d]pyrimidin-1-yl]-2-(methoxymethyl)pyrrolidin-1-yl]prop-2-en-1-one (0.18 g, 39%) as a white solid. MS ESI calculated for C₂₄H₂₃ClN₈O₂ [M+H]⁺, 491.20, found 491.10; ¹H NMR (300 MHz, DMSO-d₆) δ 8.29 (d, J=1.8 Hz, 1H), 7.95 (s, 1H), 7.70 (s, 1H), 6.66-6.32 (m, 2H), 6.16-5.96 (m, 1H), 5.67-5.31 (m, 2H), 4.73-4.40 (m, 1H), 4.18-3.73 (m, 2H), 3.70-3.43 (m, 2H), 3.32 (s, 3H), 2.75-2.58 (m, 1H), 2.50 (s, 3H), 2.38-2.12 (m, 1H).

### Example 100: 1-[(2R,4S)-4-[4-Amino-3-[2-(6-chloro-2-methyl-1H-1,3-benzodiazol-5-yl)ethynyl]pyrazolo[4,3-c]pyridin-1-yl]-2-(methoxymethyl)pyrrolidin-1-yl]prop-2-en-1-one

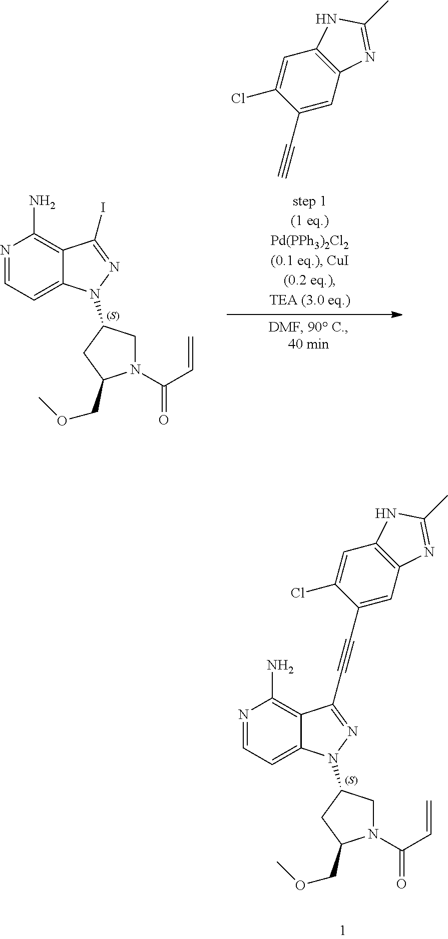

To a stirred mixture of 1-[(2R,4S)-4-[4-amino-3-iodopyrazolo[4,3-c]pyridin-1-yl]-2-(methoxymethyl)pyrrolidin-1-yl]prop-2-en-1-one (0.50 g, 1.17 mmol), 5-chloro-6-ethynyl-2-methyl-3H-1,3-benzodiazole (0.22 g, 1.17 mmol), Pd(PPh₃)₂Cl₂ (82.14 mg, 0.12 mmol) and CuI (44.58 mg, 0.23 mmol) in DMF (1.50 mL) was added TEA (0.35 g, 3.51 mmol) dropwise at room temperature. The reaction mixture was degassed with argon for three times and stirred for 40 min at 90° C. The resulting mixture was concentrated under reduced pressure. The residue was purified by silica gel column chromatography, eluted with CH₂Cl₂/MeOH (10/1). The fractions contained desired product were combined and concentrated. The crude product was purified by Prep-HPLC with the following conditions Column: XBridge Prep C18 OBD Column, 19×150 mm 5 μm; Mobile Phase A: water (10 mmol/L NH₄HCO₃), Mobile Phase B: ACN; Flow rate: 20 mL/min; Gradient: 35 B to 55 B in 6 min; 210/254 nm. The fractions contained desired product were combined and concentrated to afford 1-[(2R,4S)-4-[4-amino-3-[2-(6-chloro-2-methyl-1H-1,3-benzodiazol-5-yl)ethynyl]pyrazolo[4,3-c]pyridin-1-yl]-2-(methoxymethyl)pyrrolidin-1-yl]prop-2-en-1-one (0.21 g, 36%) as a white solid. MS ESI calculated for C₂₅H₂₄ClN₇O₂ [M+H]⁺, 490.20, found 490.05; ¹H NMR (400 MHz, DMSO-d₆) δ 12.66 (d, J=18.1 Hz, 1H), 7.99-7.86 (m, 1H), 7.81 (d, J=6.1 Hz, 1H), 7.76-7.67 (m, 1H), 6.97 (d, J=6.2 Hz, 1H), 6.86-6.46 (m, 3H), 6.17-5.96 (m, 1H), 5.79-5.64 (m, 1H), 5.50 (s, 1H), 4.48 (s, 1H), 4.10-4.01 (m, 1H), 3.97-3.75 (m, 2H), 3.67-3.43 (m, 3H), 3.30 (s, 1H), 2.55 (s, 1H), 2.52 (s, 3H), 2.40 (s, 1H).

### Example 101: 1-[(2R,4S)-4-[4-Amino-5-[2-(6-chloro-1,2-dimethyl-1,3-benzodiazol-5-yl)ethynyl]pyrrolo[2,3-d]pyrimidin-7-yl]-2-(methoxymethyl)pyrrolidin-1-yl]prop-2-en-1-one

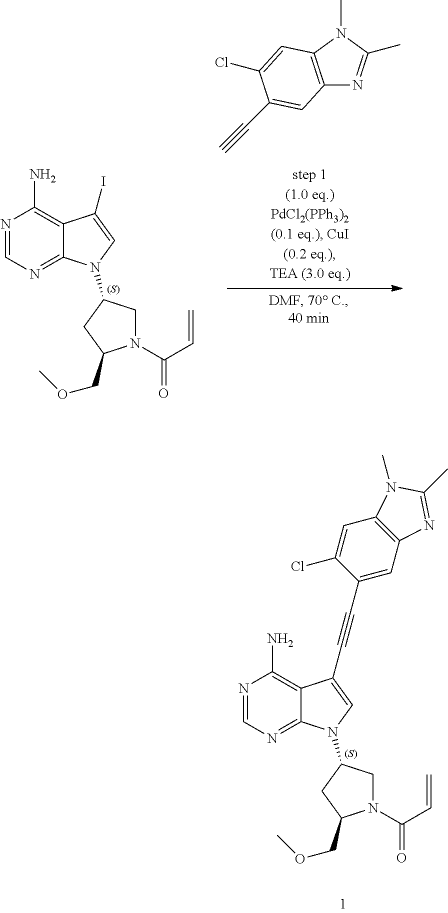

1-[(2R,4S)-4-[4-amino-5-[2-(6-chloro-1,2-dimethyl-1,3-benzodiazol-5-yl)ethynyl]pyrrolo[2,3-d]pyrimidin-7-yl]-2-(methoxymethyl)pyrrolidin-1-yl]prop-2-en-1-one. MS ESI calculated for C₂₆H₂₆ClN₇O₂ [M+H]⁺, 504.18, found 504.15; ¹H NMR (400 MHz, CDCl₃) δ 8.32 (s, 1H), 7.86 (s, 1H), 7.38 (s, 1H), 7.31 (s, 1H), 6.68-6.35 (m, 2H), 5.95 (s, 2H), 5.84-5.69 (m, 1H), 5.69-5.59 (m, 1H), 4.67 (s, 1H), 4.29-4.13 (m, 1H), 4.13-3.95 (m, 1H), 3.82 (s, 1H), 3.74 (s, 3H), 3.63-3.48 (m, 1H), 3.41 (s, 3H), 2.71-2.48 (m, 5H).

### Example 102: 1-((2R,4S)-4-(4-Amino-3-((6-chloro-4-fluoro-1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

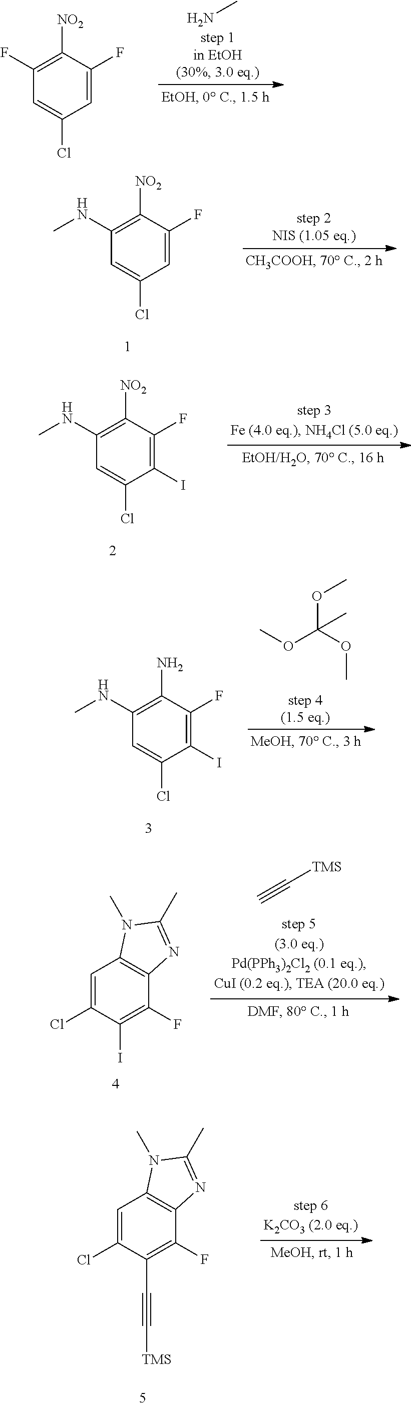

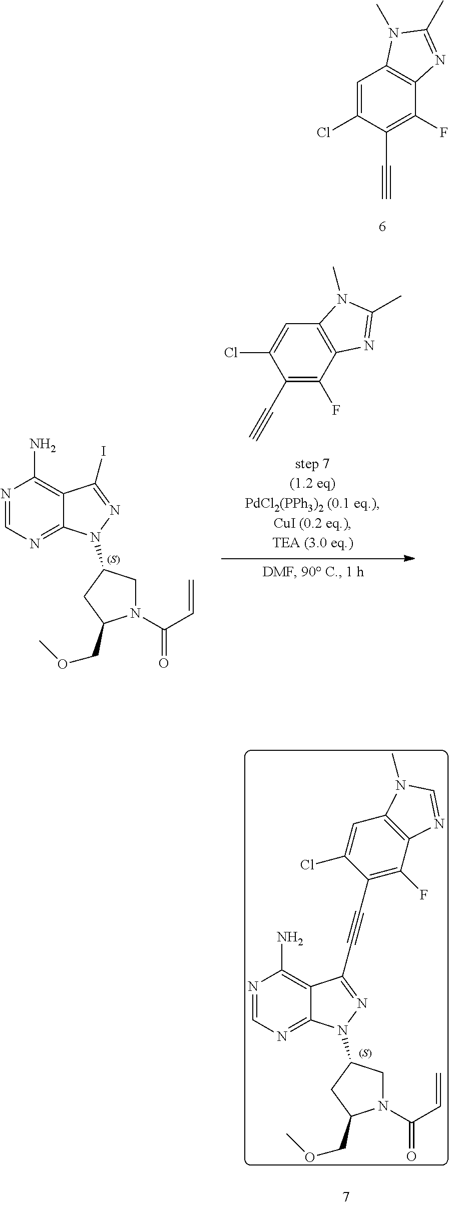

1-((2R,4S)-4-(4-amino-3-((6-chloro-4-fluoro-1,2-dimethyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one. MS ESI calculated for C₂₅H₂₄ClFN₈O₂ [M+H]⁺, 523.17, found 523.15; ¹H NMR (400 MHz, DMSO-d₆) δ 8.30 (d, J=2.3 Hz, 2H), 7.81 (s, 1H), 6.97-5.96 (m, 3H), 5.80-5.50 (m, 2H), 4.73-4.33 (m, 1H), 4.16-3.80 (m, 2H), 3.77 (s, 3H), 3.67-3.45 (m, 2H), 3.32 (s, 3H), 2.76-2.59 (m, 1H), 2.57 (s, 3H), 2.39 (d, J=5.7 Hz, 1H).

### Example 103: 1-((2R,4S)-4-(4-Amino-3-((6-chloro-4-fluoro-1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one

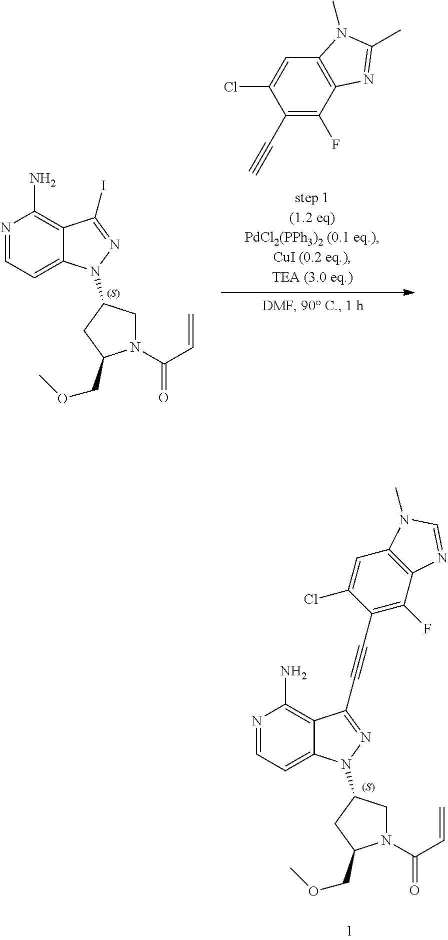

1-((2R,4S)-4-(4-amino-3-((6-chloro-4-fluoro-1,2-dimethyl-1H-benzo[d]imidazol-5-yl)ethynyl)-1H-pyrazolo[4,3-c]pyridin-1-yl)-2-(methoxymethyl)pyrrolidin-1-yl)prop-2-en-1-one. MS ESI calculated for C₂₆H₂₅ClFN₇O₂ [M+H]⁺, 522.17, found 522.15; ¹H NMR (400 MHz, DMSO-d₆) δ 7.84-7.77 (m, 2H), 6.98 (d, J=6.1 Hz, 1H), 6.83-6.46 (m, 3H), 6.16 (dd, J=16.8, 3.1 Hz, 1H), 5.69-5.65 (m, 1H), 5.50 (d, J=7.8 Hz, 1H), 4.69-4.39 (m, 1H), 4.16-3.75 (m, 5H), 3.68-3.43 (m, 2H), 3.32 (s, 3H), 2.77-2.61 (m, 1H), 2.57 (s, 3H), 2.43-2.39 (m, 1H).

### Example 104: 1-[(2R,4S)-4-[4-Amino-3-[2-(6-chloro-4-fluoro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one

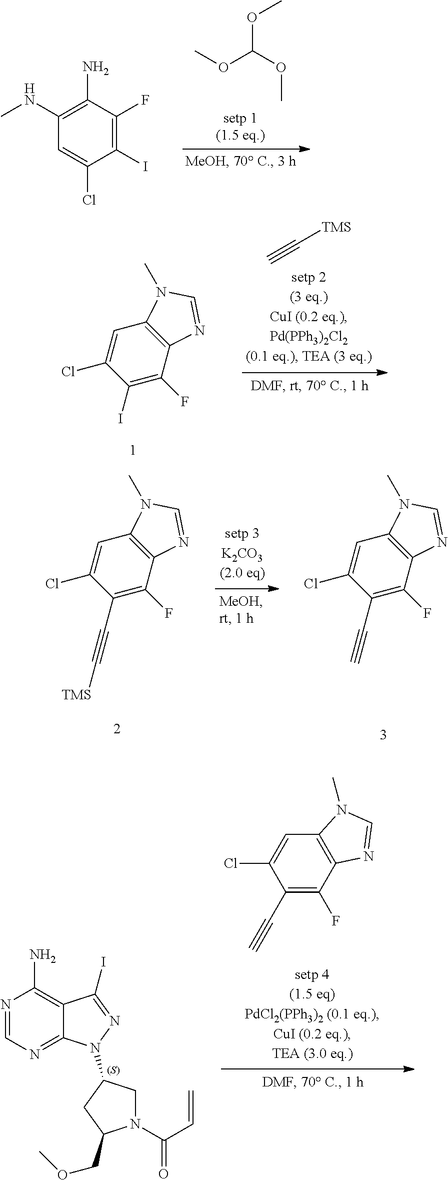

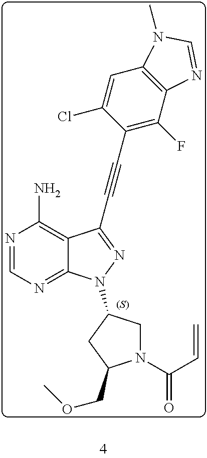

1-[(2R,4S)-4-[4-amino-3-[2-(6-chloro-4-fluoro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₄H₂₂ClFN₈O₂ [M+H]⁺, 509.15, found 509.20; ¹H NMR (400 MHz, CDCl₃) δ 8.40 (d, J=5.2 Hz, 1H), 7.95 (s, 1H), 7.38 (s, 1H), 6.51-6.37 (m, 2H), 6.32-5.95 (m, 1H), 5.92-5.80 (m, 1H), 5.78-5.68 (m, 1H), 4.77-4.46 (m, 1H), 4.24-3.98 (m, 2H), 3.89 (s, 3H), 3.87-3.80 (m, 1H), 3.59-3.49 (m, 1H), 3.42 (s, 3H), 3.05-2.78 (m, 1H), 2.57-2.45 (m, 1H).

### Example 105: 1-[(2R,4S)-4-[4-Amino-3-[2-(6-chloro-4-fluoro-1-methyl-1,3-benzodiazol-5-yl)ethynyl]pyrazolo[4,3-c]pyridin-1-yl]-2-(methoxymethyl)pyrrolidin-1-yl]prop-2-en-1-one

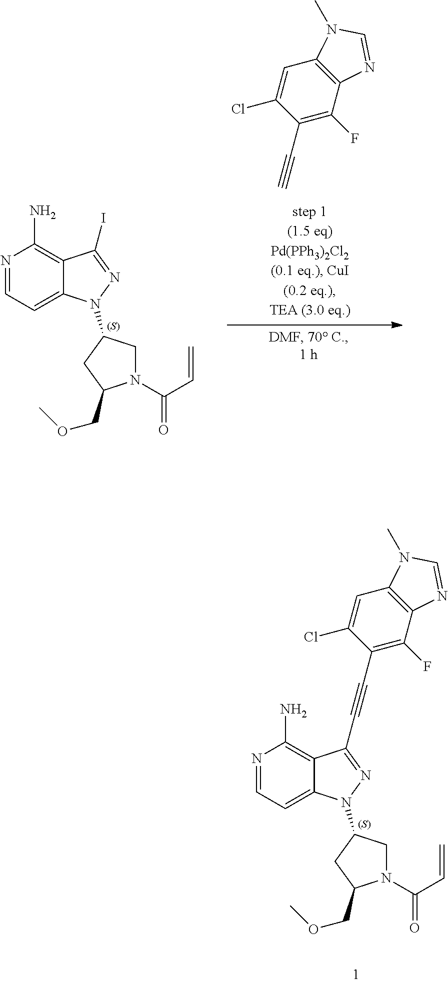

1-[(2R,4S)-4-[4-amino-3-[2-(6-chloro-4-fluoro-1-methyl-1,3-benzodiazol-5-yl)ethynyl]pyrazolo[4,3-c]pyridin-1-yl]-2-(methoxymethyl)pyrrolidin-1-yl]prop-2-en-1-one. MS ESI calculated for C₂₅H₂₃ClFN₇O₂ [M+H]⁺, 508.16, found 508.20; ¹H NMR (400 MHz, CDCl₃) δ 7.95-7.83 (m, 2H), 7.39-7.33 (m, 1H), 6.76-6.68 (m, 1H), 6.52-6.38 (m, 2H), 5.79-5.69 (m, 3H), 5.60-5.24 (m, 1H), 4.71-4.46 (m, 1H), 4.19-3.99 (m, 2H), 3.95-3.90 (m, 1H), 3.88 (s, 3H), 3.58-3.47 (m, 1H), 3.43 (s, 3H), 3.08-2.68 (m, 1H), 2.57-2.40 (m, 1H).

### Example 106: 1-[(2R,4S)-4-[4-Amino-5-[2-(6-chloro-4-fluoro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one

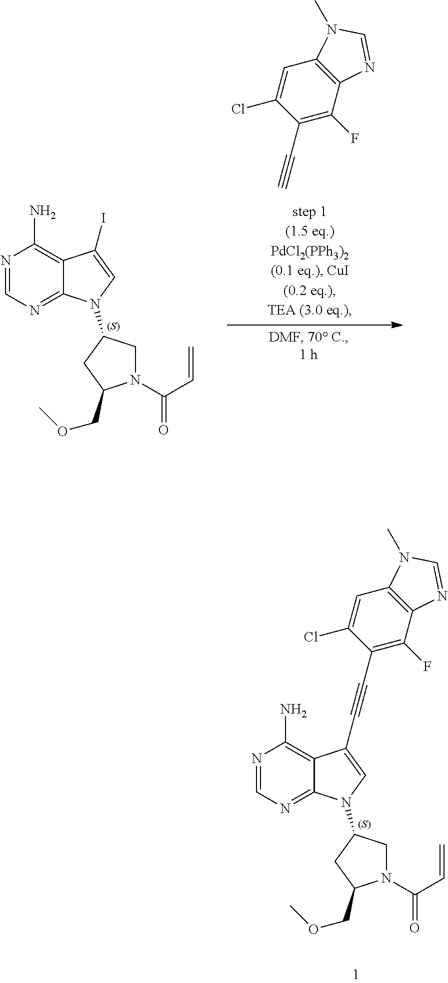

1-[(2R,4S)-4-[4-amino-5-[2-(6-chloro-4-fluoro-1-methyl-1,3-benzodiazol-5-yl)ethynyl]pyrrolo[2,3-d]pyrimidin-7-yl]-2-(methoxymethyl)pyrrolidin-1-yl]prop-2-en-1-one. MS ESI calculated for C₂₅H₂₃ClFN₇O₂ [M+H]⁺, 508.16, found 508.20; ¹H NMR (400 MHz, CDCl₃) δ 8.33 (s, 1H), 7.91 (s, 1H), 7.34 (d, J=1.6 Hz, 1H), 6.70-6.31 (m, 2H), 5.91 (s, 2H), 5.82-5.69 (m, 1H), 5.70-5.55 (m, 1H), 4.71-4.39 (m, 1H), 4.30-4.17 (m, 1H), 4.06 (d, J=7.2 Hz, 1H), 3.87 (s, 3H), 3.85-3.79 (m, 1H), 3.61-3.49 (m, 1H), 3.41 (s, 3H), 2.64-2.53 (m, 2H).

### Example 107: 1-[(2R,4S)-4-[4-Amino-5-[2-(4,6-difluoro-2-methyl-1H-1,3-benzodiazol-5-yl)ethynyl]pyrrolo[2,3-d]pyrimidin-7-yl]-2-(methoxymethyl)pyrrolidin-1-yl]prop-2-en-1-one

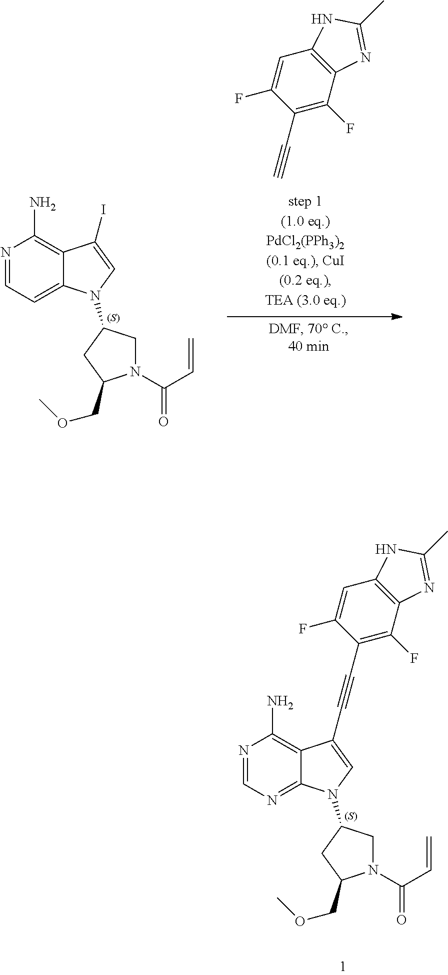

1-[(2R,4S)-4-[4-amino-5-[2-(4,6-difluoro-2-methyl-1H-1,3-benzodiazol-5-yl)ethynyl]pyrrolo[2,3-d]pyrimidin-7-yl]-2-(methoxymethyl)pyrrolidin-1-yl]prop-2-en-1-one. MS ESI calculated for C₂₅H₂₃F₂N₇O₂ [M+H]⁺, 492.19, found 492.10; ¹H NMR (400 MHz, CDCl₃) δ 8.31 (s, 1H), 7.35 (s, 1H), 7.13 (s, 1H), 6.43 (d, J=8.7 Hz, 3H), 5.79-5.52 (m, 2H), 4.69 (s, 1H), 4.28-4.19 (m, 1H), 4.09 (s, 1H), 3.90-3.82 (m, 1H), 3.57-3.52 (m, 1H), 3.42 (s, 3H), 2.69 (s, 3H), 2.57 (m, 2H).

### Example 108: 1-[(2R,4S)-4-[4-Amino-3-[2-(1-cyclopropyl-6-fluoro-1,3-benzodiazol-5-yl)ethynyl]pyrazolo[3,4-d]pyrimidin-1-yl]-2-(methoxymethyl)pyrrolidin-1-yl]prop-2-en-1-one

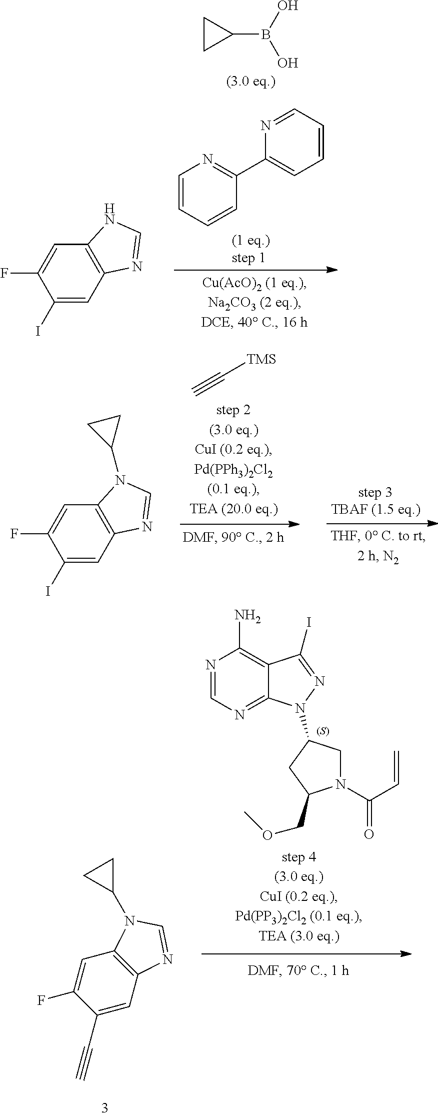

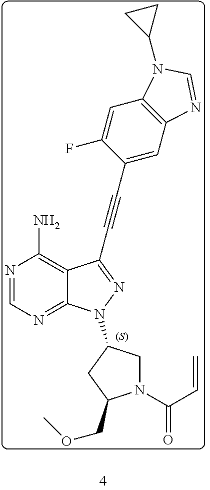

1-[(2R,4S)-4-[4-amino-3-[2-(1-cyclopropyl-6-fluoro-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₆H₂₅FN₈O₂ [M+H]⁻, 501.22, found 501.30; ¹H NMR (300 MHz, CDCl₃) δ 8.41 (s, 1H), 8.09 (s, 2H), 7.48 (s, 1H), 6.53-6.38 (m, 2H), 6.19 (s, 2H), 5.90-5.82 (m, 1H), 5.79-5.65 (m, 1H), 4.68 (s, 1H), 4.22-4.00 (m, 2H), 3.86-3.82 (m, 1H), 3.57-3.51 (m, 1H), 3.42 (s, 3H), 2.90-2.73 (m, 1H), 2.52-2.49 (m, 1H), 1.24 (d, J=6.6 Hz, 2H), 1.11-1.07 (m, 2H).

### Example 109: 1-[(2R,4S)-4-[4-Amino-3-[2-(1-cyclopropyl-6-fluoro-1,3-benzodiazol-5-yl)ethynyl]pyrazolo[4,3-c]pyridin-1-yl]-2-(methoxymethyl)pyrrolidin-1-yl]prop-2-en-1-one

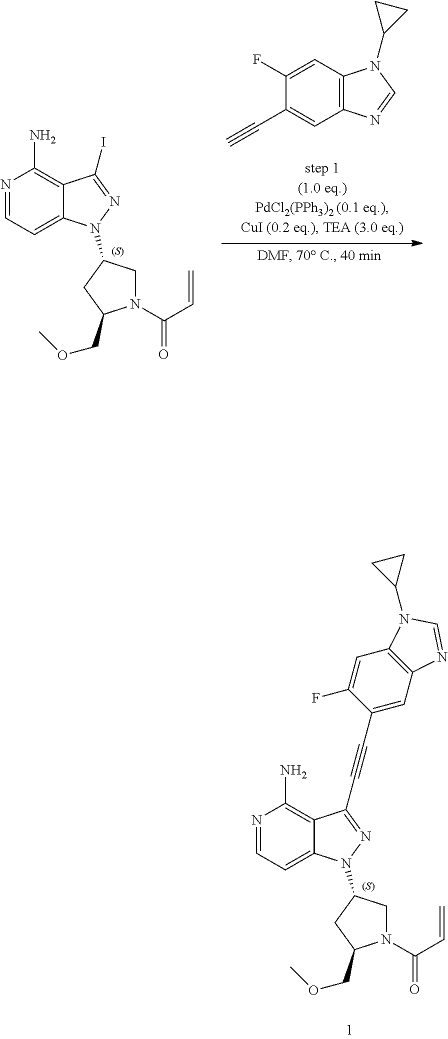

1-[(2R,4S)-4-[4-amino-3-[2-(1-cyclopropyl-6-fluoro-1,3-benzodiazol-5-yl)ethynyl]pyrazolo[4,3-c]pyridin-1-yl]-2-(methoxymethyl)pyrrolidin-1-yl]prop-2-en-1-one. MS ESI calculated for C₂₇H₂₆FN₇O₂ [M+H]⁺, 500.22, found 500.30; ¹H NMR (300 MHz, CDCl₃) δ 8.09-7.79 (m, 3H), 7.39-7.31 (m, 1H), 6.80-6.63 (m, 1H), 6.49-6.37 (m, 2H), 5.89-5.63 (m, 3H), 5.59-5.22 (m, 1H), 4.74-4.44 (m, 1H), 4.21-3.99 (m, 2H), 3.96-3.82 (m, 1H), 3.62-3.45 (m, 1H), 3.43 (s, 3H), 3.41-3.35 (m, 1H), 2.87-2.65 (m, 1H), 2.57-2.32 (m, 1H), 1.27-1.13 (m, 2H), 1.13-0.93 (m, 2H).

### Example 110: 1-[(2R,4S)-4-[4-Amino-5-[2-(1-cyclopropyl-6-fluoro-1,3-benzodiazol-5-yl)ethynyl]pyrrolo[2,3-d]pyrimidin-7-yl]-2-(methoxymethyl)pyrrolidin-1-yl]prop-2-en-1-one

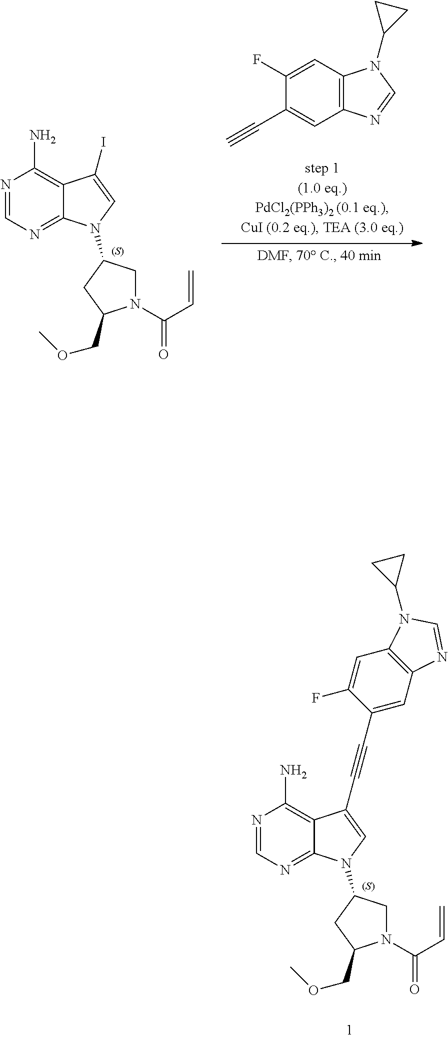

1-[(2R,4S)-4-[4-amino-5-[2-(1-cyclopropyl-6-fluoro-1,3-benzodiazol-5-yl)ethynyl]pyrrolo[2,3-d]pyrimidin-7-yl]-2-(methoxymethyl)pyrrolidin-1-yl]prop-2-en-1-one. MS ESI calculated for C₂₇H₂₆FN₇O₂ [M+H]⁺, 500.22, found 500.15; ¹H NMR (300 MHz, CDCl₃) δ 8.35 (s, 1H), 7.96 (s, 1H), 7.50-7.16 (m, 3H), 6.69-6.32 (m, 2H), 5.93 (s, 2H), 5.83-5.68 (m, 1H), 5.68-5.49 (m, 1H), 4.74-4.34 (m, 1H), 4.27-4.01 (m, 1H), 3.88-3.46 (m, 3H), 3.41 (s, 4H), 2.57 (s, 2H), 1.30-1.12 (m, 2H), 1.08 (s, 2H).

### Example 112: 1-((2R,4S)-4-(4-Amino-3-(benzo[d]thiazol-5-ylethynyl)-1H-pyrazolo[3,4-d]pyrimidin-1-yl)-2-(methoxymethyl)pyrrolidin-1-yl)prop-2-en-1-one

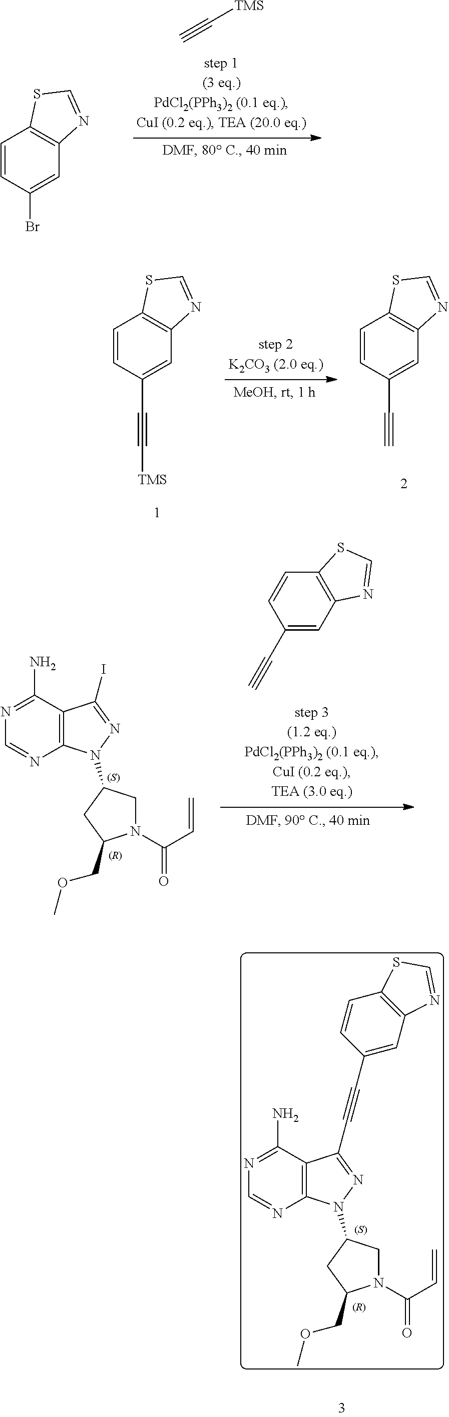

1-((2R,4S)-4-(4-amino-3-(benzo[d]thiazol-5-ylethynyl)-1H-pyrazolo[3,4-d]pyrimidin-1-yl)-2-(methoxymethyl)pyrrolidin-1-yl)prop-2-en-1-one. MS ESI calculated for C₂₃H₂₁N₇O₂S [M+H]⁺, 460.15, found 460.10; ¹H NMR (400 MHz, DMSO-d₆) δ 9.50 (s, 1H), 8.56-8.51 (m, 1H), 8.31-8.24 (m, 2H), 7.80 (dd, J=8.4, 1.6 Hz, 1H), 6.69-6.64 (m, 1H), 6.17-6.10 (m, 1H), 5.75-5.57 (m, 2H), 4.54-4.45 (m, 1H), 4.24-3.75 (m, 2H), 3.70-3.42 (m, 2H), 3.33 (s, 3H), 2.74-2.56 (m, 1H), 2.45-2.34 (m, 1H).

### Example 113: 1-((2R,4S)-4-(4-Amino-5-((6-chloro-4-fluoro-1,2-dimethyl-1H-benzo[d]imidazol-5-yl)ethynyl)-7H-pyrrolo[2,3-d]pyrimidin-7-yl)-2-(methoxymethyl)pyrrolidin-1-yl)prop-2-en-1-one

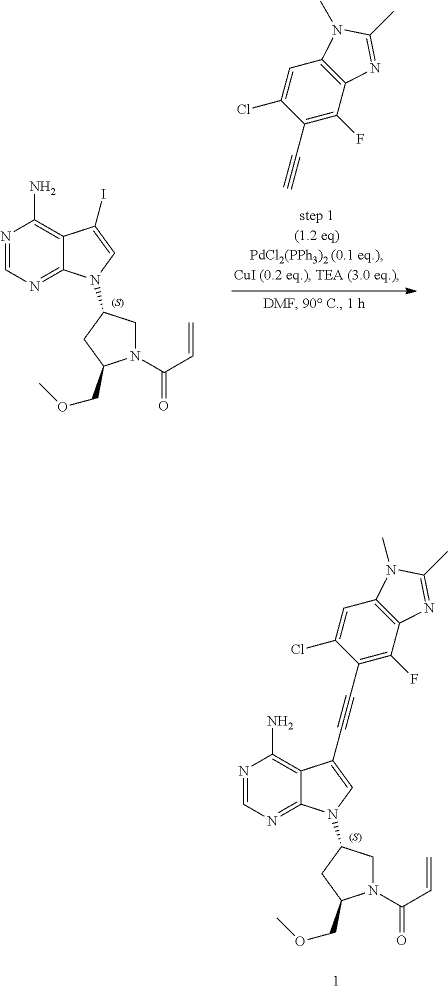

1-((2R,4S)-4-(4-amino-5-((6-chloro-4-fluoro-1,2-dimethyl-1H-benzo[d]imidazol-5-yl)ethynyl)-7H-pyrrolo[2,3-d]pyrimidin-7-yl)-2-(methoxymethyl)pyrrolidin-1-yl)prop-2-en-1-one. MS ESI calculated for C₂₆H₂₅ClFN₇O₂ [M+H]⁺, 522.17, found 522.20; H NMR (400 MHz, DMSO-d₆) δ 8.19 (d, J=4.1 Hz, 1H), 7.89 (d, J=22.0 Hz, 1H), 7.76 (s, 1H), 6.76 (dd, J=16.7, 10.3 Hz, 1H), 6.57 (dd, J=16.7, 10.3 Hz, 1H), 6.19-6.17 (m, 1H), 5.80-5.64 (m, 1H), 5.52-5.48 (m, 1H), 4.66-4.38 (m, 1H), 4.14-3.80 (m, 2H), 3.76 (s, 3H), 3.66-3.40 (m, 2H), 3.32 (s, 3H), 2.72-2.60 (m, 1H), 2.56 (s, 3H), 2.37-2.33 (m, 1H).

### Example 114: 1-[(2R,4S)-4-[4-Amino-3-(2-[[1,2,4]triazolo[1,5-a]pyridin-7-yl]ethynyl)pyrazolo[3,4-d]pyrimidin-1-yl]-2-(methoxymethyl)pyrrolidin-1-yl]prop-2-en-1-one

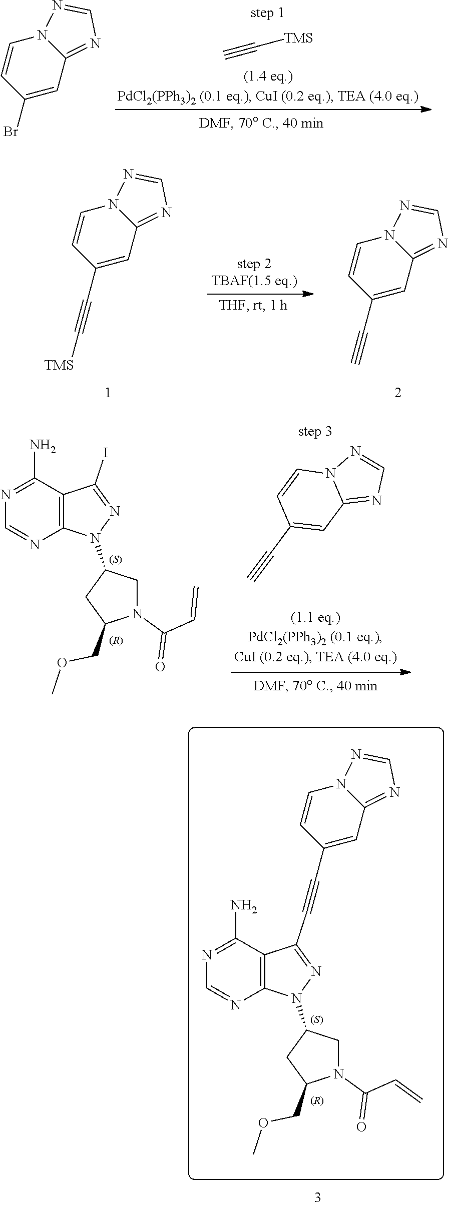

1-[(2R,4S)-4-[4-amino-3-(2-[[1,2,4]triazolo[1,5-a]pyridin-7-yl]ethynyl)pyrazolo[3,4-d]pyrimidin-1-yl]-2-(methoxymethyl)pyrrolidin-1-yl]prop-2-en-1-one. MS ESI calculated for C₂₂H₂₁N₉O₂ [M+H]⁺, 444.18, found 444.30; ¹H NMR (300 MHz, DMSO-d₆) δ 9.09-9.01 (m, 1H), 8.63 (s, 1H), 8.40 (s, 1H), 8.31 (d, J=1.8 Hz, 1H), 7.50 (dd, J=7.1, 1.7 Hz, 1H), 6.77-6.58 (m, 1H), 6.25-6.11 (m, 1H), 5.77-5.59 (m, 2H), 4.62-4.59 (m, 1H), 4.12-3.83 (m, 2H), 3.64-3.42 (m, 2H), 3.32-3.29 (m, 3H), 2.75-2.52 (m, 2H).

### Example 115: 1-[(2R,4S)-4-[4-Amino-3-[2-(1,3-benzoxazol-5-yl)ethynyl]pyrazolo[3,4-d]pyrimidin-1-yl]-2-(methoxymethyl)pyrrolidin-1-yl]prop-2-en-1-one

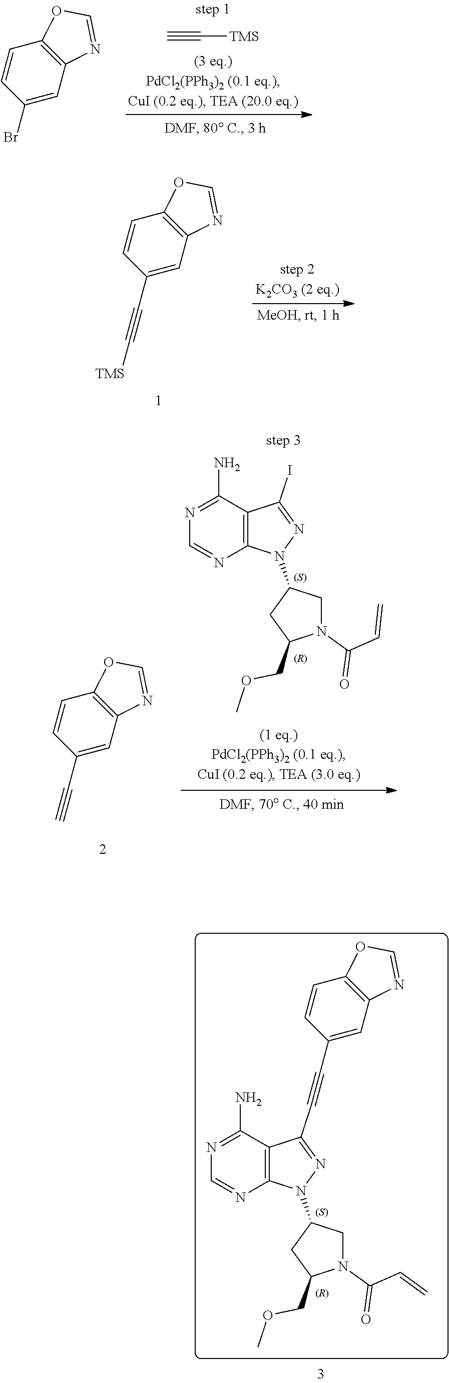

1-[(2R,4S′)-4-[4-amino-3-[2-(1,3-benzoxazol-5-yl)ethynyl]pyrazolo[3,4-d]pyrimidin-1-yl]-2-(methoxymethyl)pyrrolidin-1l-yl]prop-2-en-1-one. MS ESI calculated for C₂₃H₂₁N₇O₃ [M+H]⁺, 444.20, found 444.15; ¹H NMR (400 MHz, CDCl₃) δ 8.41 (d, J=5.3 Hz, 1H), 8.20 (s, 1H), 8.08-8.05 (m, 1H), 7.66-6.96 (m, 2H), 6.73-6.35 (m, 2H), 5.96 (s, 2H), 5.91-5.68 (m, 2H), 4.79-4.46 (m, 1H), 4.23-3.95 (m, 2H), 3.91-3.49 (m, 2H), 3.42 (s, 3H), 2.89-2.66 (m, 1H), 2.51-2.32 (in, 1H).

### Example 116: 1-[(2R,4S)-4-[4-Amino-3-[2-(6-chloro-1-ethyl-4-fluoro-1,3-benzodiazol-5-yl)ethynyl]pyrazolo [3,4-d] pyrimidin-1-yl]-2-(methoxymethyl)pyrrolidin-1-yl]prop-2-en-1-one

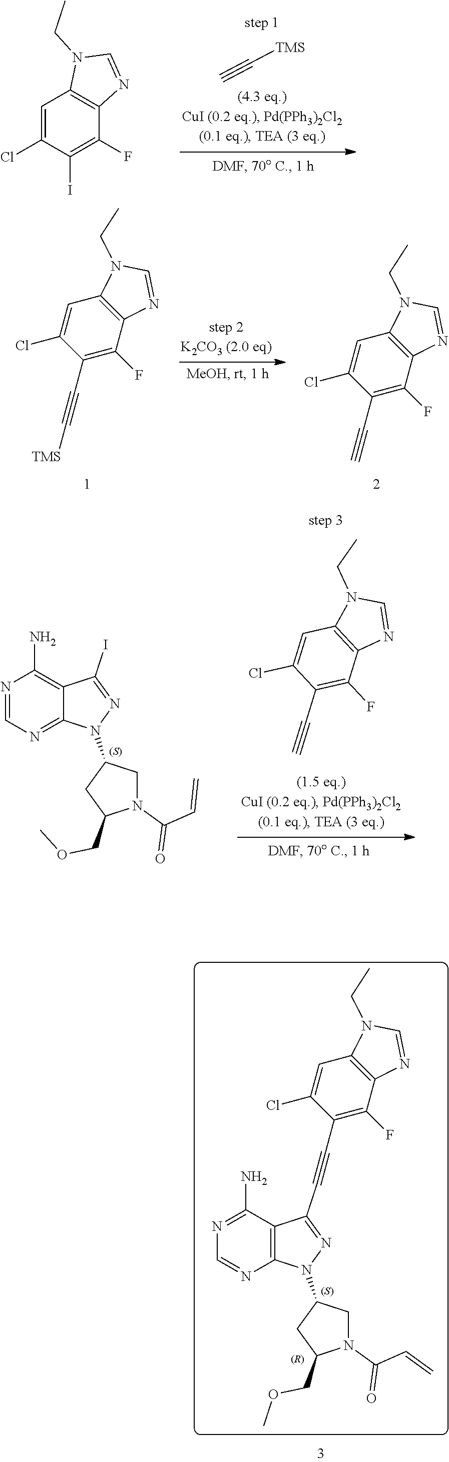

1-[(2R,4S)-4-[4-amino-3-[2-(6-chloro-1-ethyl-4-fluoro-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₅H₂₄ClFN₈O₂ [M+H]⁺, 523.17, found 523.25; ¹H NMR (400 MHz, CDCl₃) δ 8.40 (d, J=5.6 Hz, 1H), 7.99 (d, J=2.0 Hz, 1H), 7.38 (d, J=2.4 Hz, 1H), 6.67-6.45 (m, 1H), 6.46-6.09 (m, 2H), 5.92-5.79 (m, 1H), 5.78-5.67 (m, 1H), 4.75-4.46 (m, 1H), 4.30-3.97 (m, 4H), 3.88-3.79 (m, 1H), 3.59-3.49 (m, 1H), 3.42 (s, 3H), 3.07-2.76 (m, 1H), 2.56-2.45 (m, 1H), 1.60 (t, J=7.2 Hz, 3H).

### Example 117: 1-[(2R,4S)-4-[4-Amino-3-[2-(6-chloro-1-ethyl-4-fluoro-1,3-benzodiazol-5-yl)ethynyl]pyrazolo[4,3-c]pyridin-1-yl]-2-(methoxymethyl)pyrrolidin-1-yl]prop-2-en-1-one

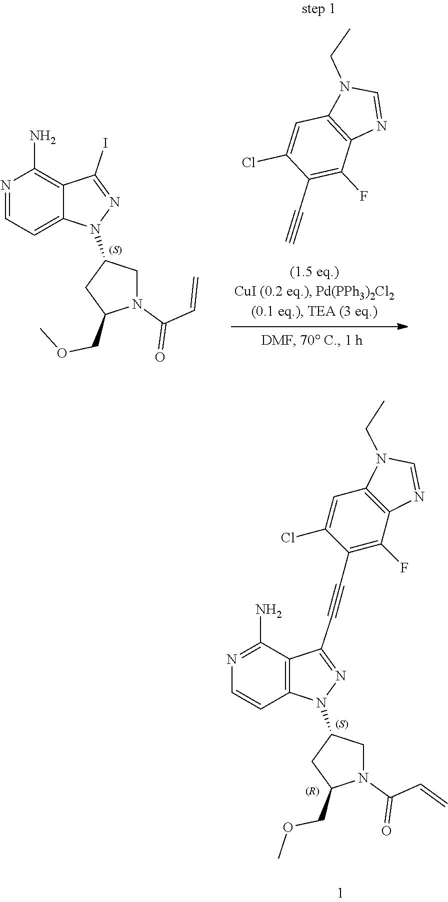

1-[(2R,4S)-4-[4-amino-3-[2-(6-chloro-1-ethyl-4-fluoro-1,3-benzodiazol-5-yl)ethynyl]pyrazolo[4,3-c]pyridin-1-yl]-2-(methoxymethyl)pyrrolidin-1-yl]prop-2-en-1-one. MS ESI calculated for C₂₆H₂₅ClFN₇O₂ [M+H]⁺, 522.17, found 522.30; ¹H NMR (400 MHz, CDCl₃) δ 7.97 (d, J=2.0 Hz, 1H), 7.91-7.86 (m, 1H), 7.37 (d, J=2.8 Hz, 1H), 6.76-6.68 (m, 1H), 6.49-6.38 (m, 2H), 5.82-5.69 (m, 3H), 5.59-5.46 (m, 1H), 4.71-4.45 (m, 1H), 4.28-4.21 (m, 2H), 4.14 (d, J=8.4 Hz, 2H), 4.07-3.88 (m, 1H), 3.58-3.47 (m, 1H), 3.43 (s, 3H), 3.06-2.71 (m, 1H), 2.57-2.40 (m, 1H), 1.59 (t, J=7.2 Hz, 3H).

### Example 118: 1-1-[(2R,4S)-4-[4-Amino-5-[2-(6-chloro-1-ethyl-4-fluoro-1,3-benzodiazol-5-yl)ethynyl]pyrrolo[2,3-d]pyrimidin-7-yl]-2-(methoxymethyl)pyrrolidin-1-yl]prop-2-en-1-one

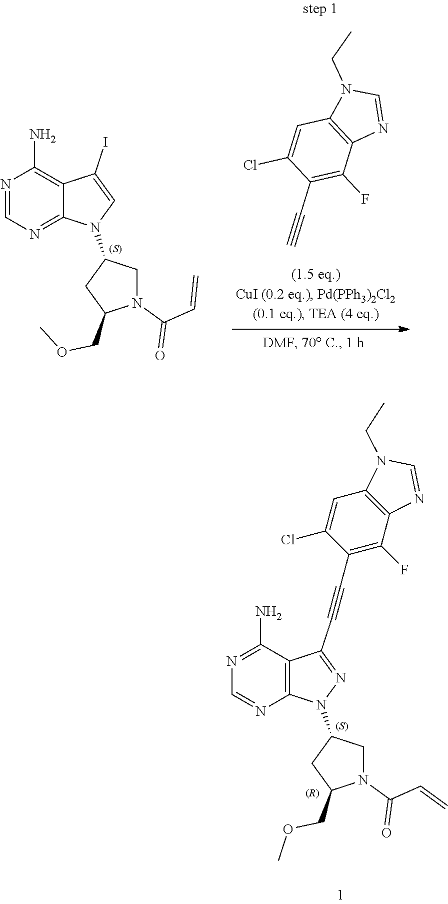

1-[(2R,4S)-4-[4-amino-5-[2-(6-chloro-1-ethyl-4-fluoro-1,3-benzodiazol-5-yl)ethynyl]pyrrolo[2,3-d]pyrimidin-7-yl]-2-(methoxymethyl)pyrrolidin-1-yl]prop-2-en-1-one. MS ESI calculated for C₂₆H₂₅ClFN₇O₂ [M+H]⁺, 522.17, found 522.25; ¹H NMR (400 MHz, CDCl₃) δ 8.33 (s, 1H), 7.96 (s, 1H), 7.35 (s, 2H), 6.71-6.37 (m, 2H), 6.00 (s, 2H), 5.81-5.56 (m, 2H), 4.74-4.37 (m, 1H), 4.29-4.18 (m, 3H), 4.10-4.03 (m, 1H), 3.87-3.77 (m, 1H), 3.61-3.49 (m, 1H), 3.41 (s, 3H), 2.64-2.54 (m, 2H), 1.59 (t, J=7.2 Hz, 3H).

### Example 119: 1-[(2R,4S)-4-[4-Amino-3-[2-(6-fluoro-1,3-benzoxazol-5-yl)ethynyl]pyrazolo[3,4-d]pyrimidin-1-yl]-2-(methoxymethyl)pyrrolidin-1-yl]prop-2-en-1-one

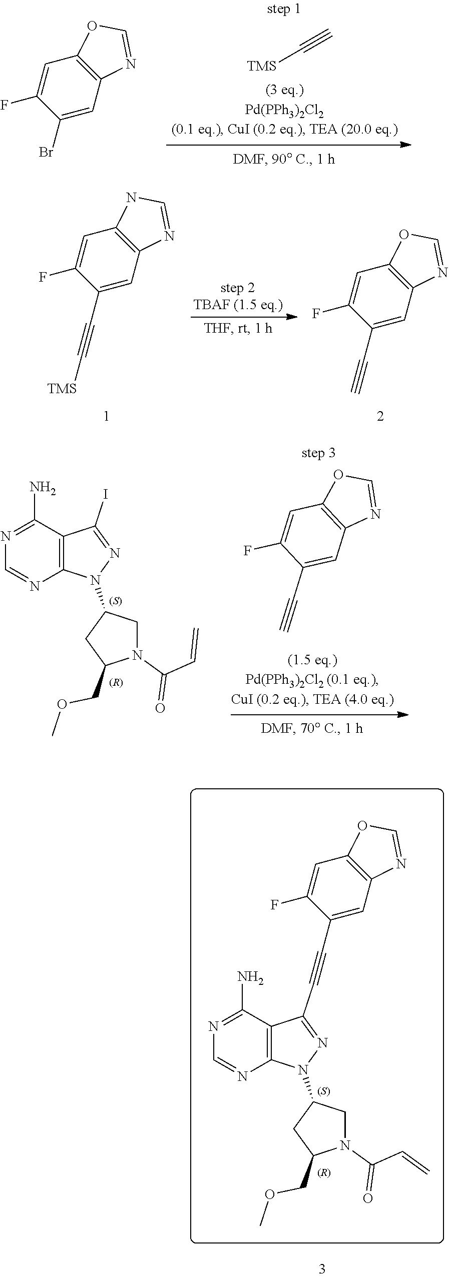

1-[(2R,4S)-4-[4-amino-3-[2-(6-fluoro-1,3-benzoxazol-5-yl)ethynyl]pyrazolo[3,4-d]pyrimidin-1-yl]-2-(methoxymethyl)pyrrolidin-1-yl]prop-2-en-1-one. MS ESI calculated for C₂₃H₂₀FN₇O₃[M+H]⁺, 462.46, found 462.16; ¹H NMR (400 MHz, CDCl₃) δ 8.41 (d, J=4 Hz, 1H), 8.18 (s, 1H), 8.04 (t, J=24 Hz, 1H), 7.46 (d, J=8 Hz, 1H), 6.60-6.42 (m, 2H), 6.10 (s, 2H), 5.88-5.83 (m, 1H), 5.76-5.70 (m, 1H), 4.70-4.52 (m, 1H), 4.19-4.03 (m, 2H), 3.87-3.84 (m, 1H), 3.57-3.52 (m, 1H), 3.42 (s, 3H), 2.95-2.77 (m, 1H), 2.54-2.49 (m, 1H).

### Example 120: 1-((2R,4S)-4-(4-Amino-3-((6,7-difluoro-1-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one

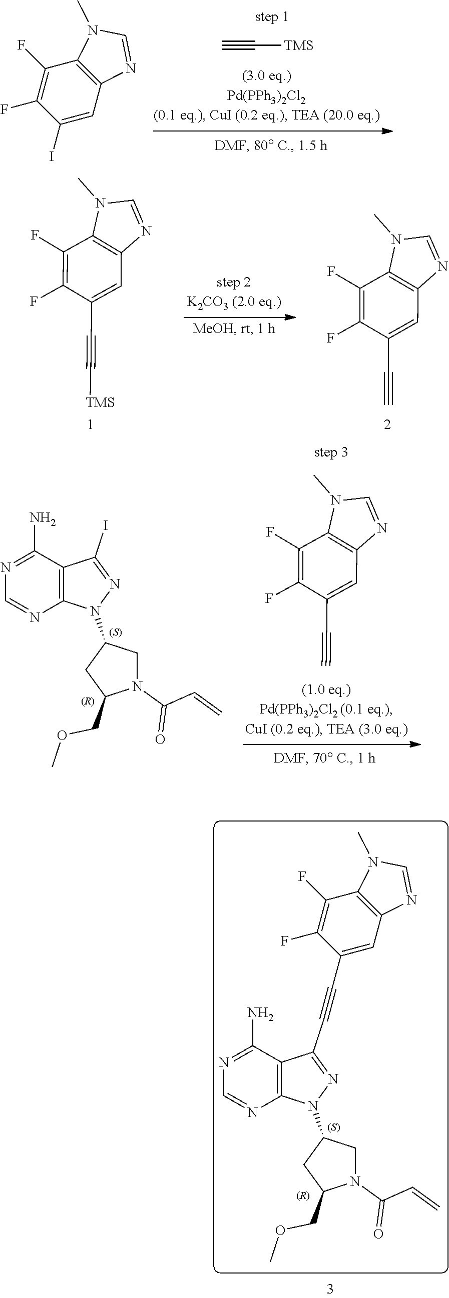

1-((2R,4S)-4-(4-amino-3-((6,7-difluoro-1-methyl-1H-benzo[d]imidazol-5-yl)ethynyl)-1H-pyrazolo[3,4-d]pyrimidin-1-yl)-2-(methoxymethyl)pyrrolidin-1-yl)prop-2-en-1-one. MS ESI calculated for C₂₄H₂₂F₂N₈O₂ [M+H]⁺, 493.18, found 493.20; ¹H NMR (300 MHz, DMSO-d₆) δ 8.51-8.16 (m, 2H), 8.03 (d, J=5.2 Hz, 1H), 6.35-6.81 (m, 1H), 6.17 (dd, J=16.7, 2.4 Hz, 1H), 5.83-5.49 (m, 2H), 4.55 (d, J=40.2 Hz, 1H), 4.27-3.75 (m, 5H), 3.70-3.41 (m, 2H), 3.34 (s, 3H), 2.85-2.56 (m, 1H), 2.42 (s, 1H).

### Example 121: 1-[(2R,4S)-4-[4-Amino-3-[2-(6-chloro-7-fluoro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one

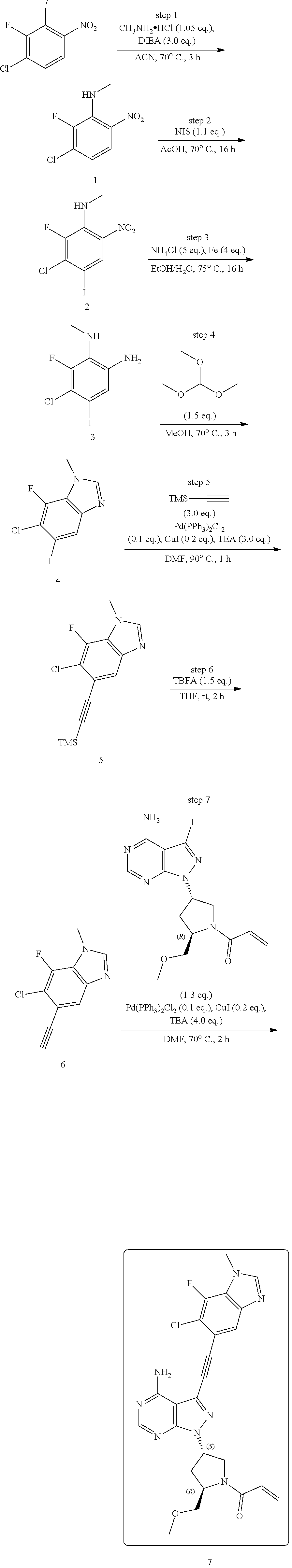

1-[(2R,4S)-4-[4-amino-3-[2-(6-chloro-7-fluoro-1-methyl-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₄H₂₂ClFN₈O₂ [M+H]⁺, 509.15, found 509.25; ¹H NMR (400 MHz, CDCl₃) δ 8.41 (d, J=5.3 Hz, 1H), 7.98-7.91 (m, 2H), 6.70-6.36 (m, 2H), 6.20-6.10 (m, 2H), 5.90-5.70 (m, 2H), 4.42-4.00 (m, 6H), 3.87-3.83 (m, 1H), 3.57-3.52 (m, 1H), 3.42 (s, 3H), 3.12-2.70 (m, 1H), 2.53-2.48 (m, 1H).

### Example 122: 1-[(2R,4S)-4-[4-Amino-3-[2-(6,7-difluoro-1,2-dimethyl-1,3-benzodiazol-5-yl)ethynyl]pyrazolo[3,4-d]pyrimidin-1-yl]-2-(methoxymethyl)pyrrolidin-1-yl]prop-2-en-1-one

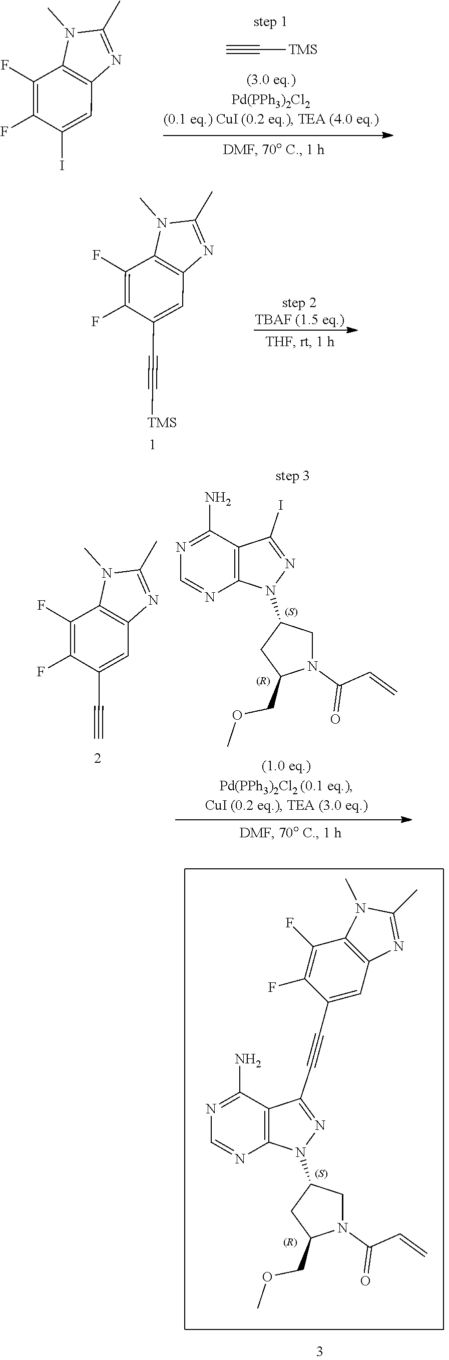

1-[(2R,4S)-4-[4-amino-3-[2-(6,7-difluoro-1,2-dimethyl-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₅H₂₄F₂N₈O₂ [M+H]⁺, 507.52, found 507.20; ¹H NMR (400 MHz, CDCl₃) δ 8.41 (s, 1H), 7.67 (d, J=4 Hz, 1H), 6.60-6.42 (m, 2H), 6.09 (s, 2H), 5.86-5.69 (m, 2H), 4.67-4.52 (m, 1H), 4.19-4.03 (m, 2H), 3.97 (s, 3H), 3.87-3.82 (m, 1H), 3.57-3.53 (m, 1H), 3.42 (s, 3H), 2.96-2.62 (m, 1H), 2.62 (s, 3H), 2.54-2.47 (m, 1H).

### Example 123: 1-[(2R,4S)-4-[4-Amino-3-[2-(1-ethyl-6,7-difluoro-1,3-benzodiazol-5-yl)ethynyl]pyrazolo[3,4-d]pyrimidin-1-yl]-2-(methoxymethyl)pyrrolidin-1-yl]prop-2-en-1-one

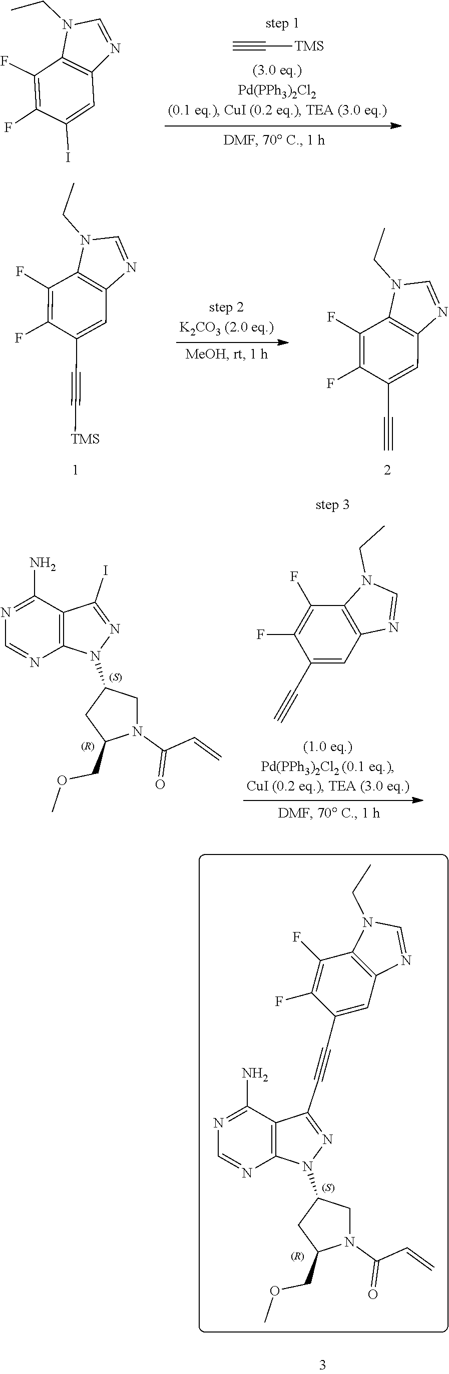

1-[(2R,4S)-4-[4-amino-3-[2-(1-ethyl-6,7-difluoro-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₅H₂₄F₂N₈O₂ [M+H]⁺, 507.20, found 507.30; ¹H NMR (400 MHz, CDCl₃) δ 8.39 (d, J=5.5 Hz, 1H), 7.92 (s, 1H), 7.80 (d, J=5.3 Hz, 1H), 6.48-6.35 (m, 2H), 6.09 (s, 2H), 5.90-5.77 (m, 1H), 5.71-5.70 (m, 1H), 4.70-4.63 (m, 1H), 4.39-4.38 (m, 2H), 4.14-4.11 (m, 1H), 4.10-3.95 (m, 1H), 3.83-3.79 (m, 1H), 3.57-3.48 (m, 1H), 3.40 (s, 3H), 2.79-2.78 (m, 1H), 2.49-2.48 (m, 1H), 1.63-1.55 (m, 3H).

### Example 124: 1-((2R,4S)-4-(4-Amino-3-((6-fluoroimidazo[1,2-a]pyridin-7-yl)ethynyl)-1H-pyrazolo[3,4-d]pyrimidin-1-yl)-2-(methoxymethyl)pyrrolidin-1-yl)prop-2-en-1-one

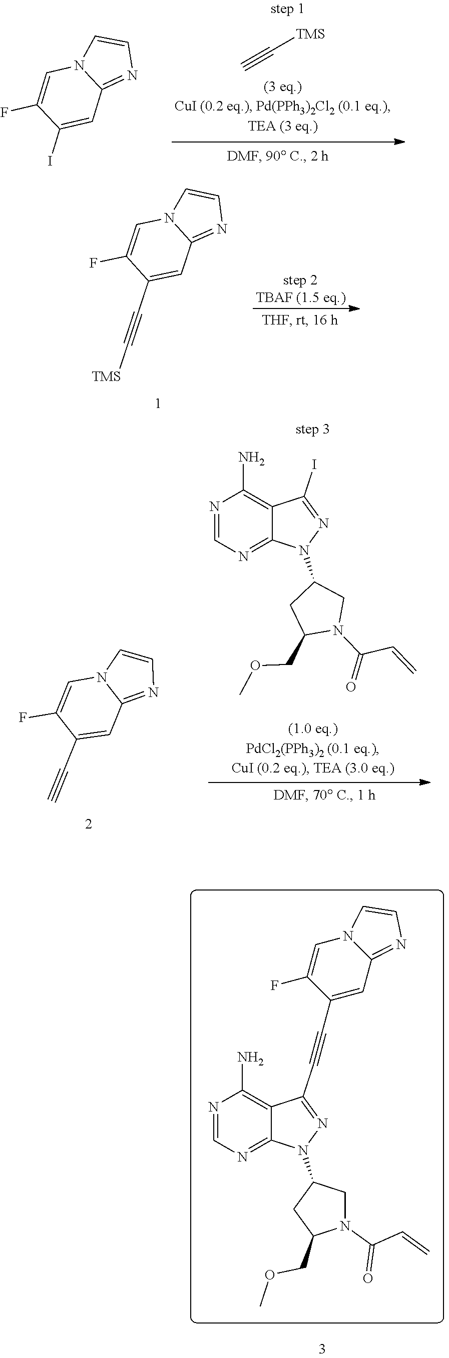

1-((2R,4S)-4-(4-amino-3-((6-fluoroimidazo[1,2-a]pyridin-7-yl)ethynyl)-1H-pyrazolo[3,4-d]pyrimidin-1-yl)-2-(methoxymethyl)pyrrolidin-1-yl)prop-2-en-1-one. MS ESI calculated for C₂₃H₂₁FN₈O₂ [M+H]⁺, 461.18, found 461.15; ¹H NMR (300 MHz, DMSO-d₆) δ 8.94 (d, J=5.1 Hz, 1H), 8.36-8.23 (m, 2H), 8.07 (s, 1H), 7.80 (s, 1H), 6.51-6.87 (m, 1H), 6.10-6.23 (m, 1H), 5.78-5.55 (m, 2H), 4.55 (d, J=40.6 Hz, 1H), 4.18-3.77 (m, 2H), 3.72-3.45 (m, 2H), 3.33 (s, 3H), 2.79-2.54 (m, 1H), 2.47-2.35 (m, 1H).

### Example 125: 1-[(2R,4S)-4-[4-Amino-3-[2-(6-chloro-7-fluoro-1,2-dimethyl-1,3-benzodiazol-5-yl)ethynyl]pyrazolo[3,4-d]pyrimidin-1-yl]-2-(methoxymethyl)pyrrolidin-1-yl]prop-2-en-1-one

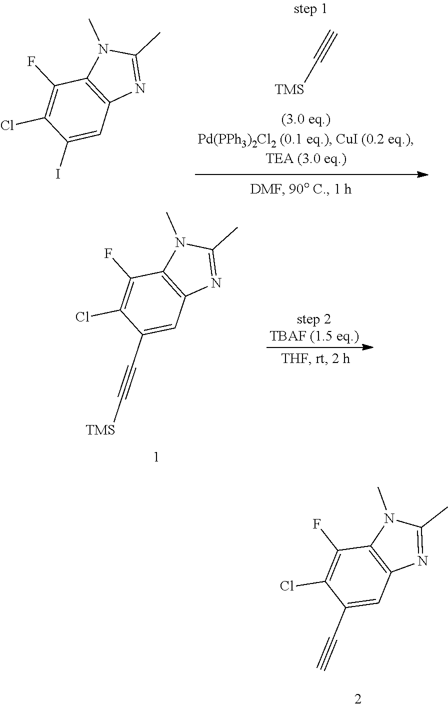

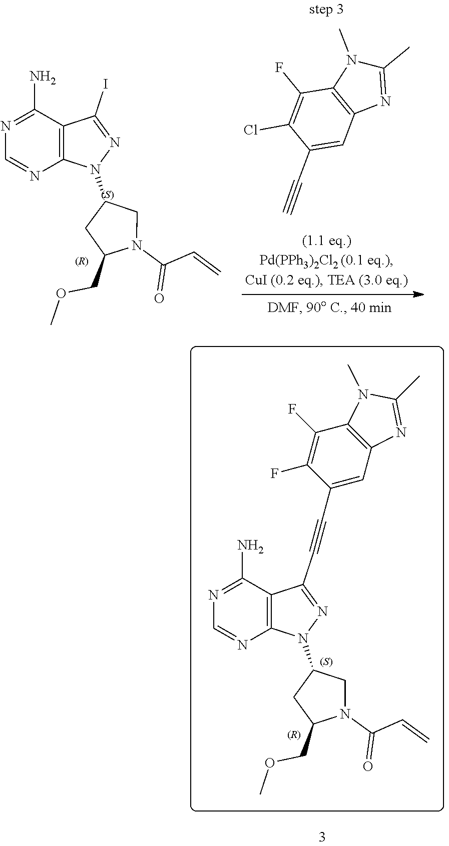

1-[(2R,4S)-4-[4-amino-3-[2-(6-chloro-7-fluoro-1,2-dimethyl-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₅H₂₄ClFN₈O₂ [M+H]⁺, 523.17, found 523.15; ¹H NMR (400 MHz, CDCl₃) δ 8.38 (d, J=5.4 Hz, 1H), 7.78 (d, J=6.1 Hz, 1H), 6.47-6.34 (m, 2H), 6.20 (s, 2H), 5.88-5.83 (m, 1H), 5.72-5.67 (m, 1H), 4.66-4.56 (m, 1H), 4.20-4.04 (m, 2H), 3.95 (s, 3H), 3.85-3.82 (m, 1H), 3.56-3.51 (m, 1H), 3.40 (s, 3H), 3.02-2.77 (m, 1H), 2.61 (s, 3H), 2.54-2.40 (m, 1H).

### Example 126: 1-[(2R,4S)-4-[4-Amino-3-[2-(6-chloro-1-cyclopropyl-1,3-benzodiazol-5-yl)ethynyl]pyrazolo[3,4-d]pyrimidin-1-yl]-2-(methoxymethyl)pyrrolidin-1-yl]prop-2-en-1-one

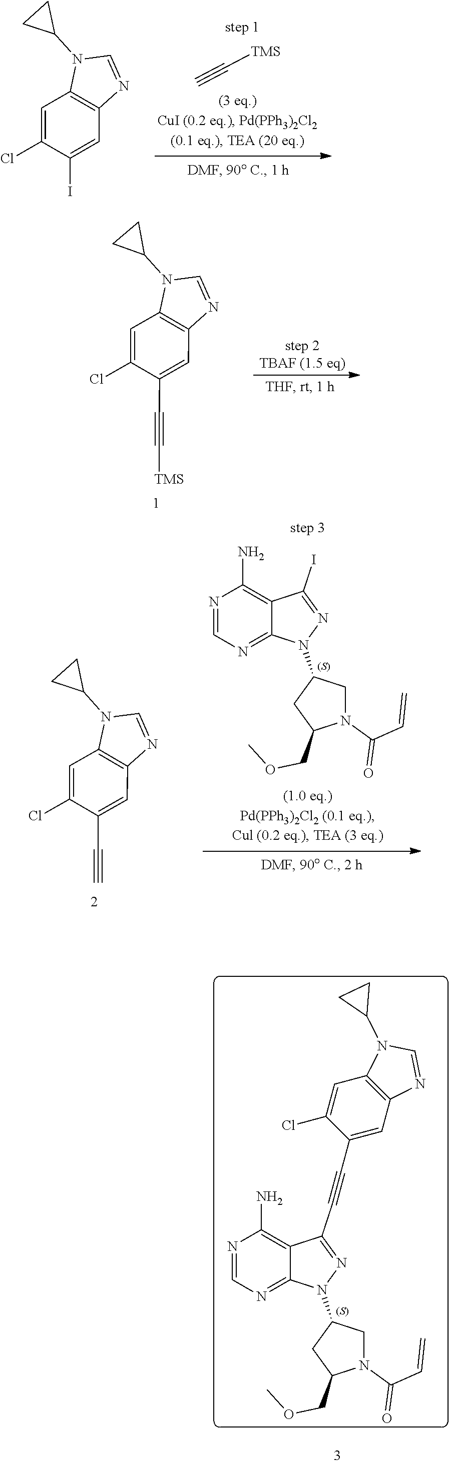

1-[(2R,4S)-4-[4-amino-3-[2-(6-chloro-1-cyclopropyl-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₆H₂₅ClN₈O₂ [M+H]⁺, 517.18, found 517.15; ¹H NMR (400 MHz, CDCl₃) δ 8.38 (d, J=5.8 Hz, 1H), 8.09 (d, J=7.0 Hz, 1H), 7.99 (d, J=2.1 Hz, 1H), 7.68 (d, J=2.0 Hz, 1H), 6.49-6.35 (m, 2H), 6.18 (s, 2H), 5.85-5.83 (m, 1H), 5.72-5.70 (m, 1H), 4.74-4.37 (m, 1H), 4.20-4.10 (m, 1H), 4.12-3.96 (m, 1H), 3.86-3.82 (m, 1H), 3.55-3.54 (m, 1H), 3.40 (s, 3H), 3.39-3.37 (m, 1H), 2.83-2.80 (m, 1H), 2.50-2.48 (m, 1H), 1.28-1.19 (m, 2H), 1.19-1.03 (m, 2H).

### Example 127: 1-[(2R,4S)-4-[4-Amino-5-[2-(6-chloro-1-cyclopropyl-1,3-benzodiazol-5-yl)ethynyl]pyrrolo[2,3-d] pyrimidin-7-yl]-2-(methoxymethyl) pyrrolidin-1-yl] prop-2-en-1-one

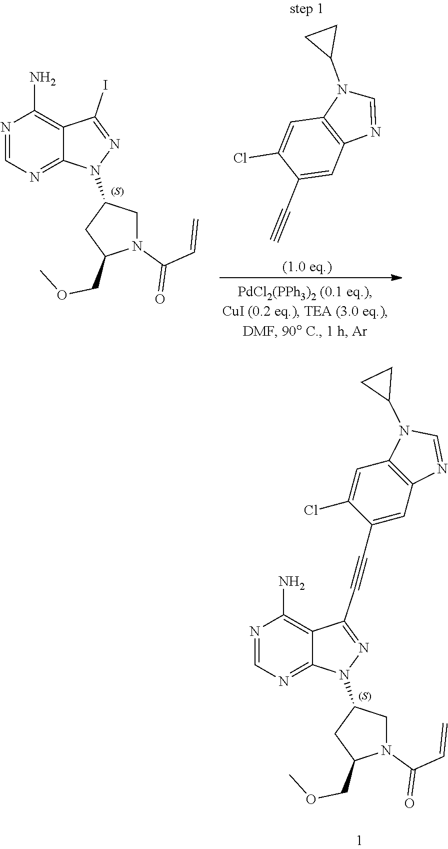

1-[(2R,4S)-4-[4-amino-5-[2-(6-chloro-1-cyclopropyl-1,3-benzodiazol-5-yl)ethynyl]pyrrolo[2,3-d]pyrimidin-7-yl]-2-(methoxymethyl) pyrrolidin-1-yl]prop-2-en-1-one. MS ESI calculated for C₂₇H₂₆ClN₇O₂ [M+H]⁺, 516.18, found 516.20; ¹H NMR (300 MHz, DMSO-d₆) δ 8.39 (s, 1H), 8.21 (s, 1H), 8.01 (s, 1H), 7.91-7.78 (m, 2H), 6.83-6.54 (m, 4H), 6.24-6.16 (m, 1H), 5.75-5.68 (m, 1H), 5.59-5.45 (m, 1H), 4.72-4.36 (m, 1H), 4.18-3.77 (m, 1H), 3.70-3.41 (m, 3H), 3.34 (s, 3H), 2.76-2.55 (m, 1H), 2.45-2.30 (m, 1H), 1.14-1.08 (m, 4H).

### Example 128: 1-[(2R,4S)-4-[4-amino-3-[2-(6-chloro-1-ethyl-7-fluoro-1,3-benzodiazol-5-yl)ethynyl]pyrazolo[3,4-d]pyrimidin-1-yl]-2-(methoxymethyl)pyrrolidin-1-yl]prop-2-en-1-one

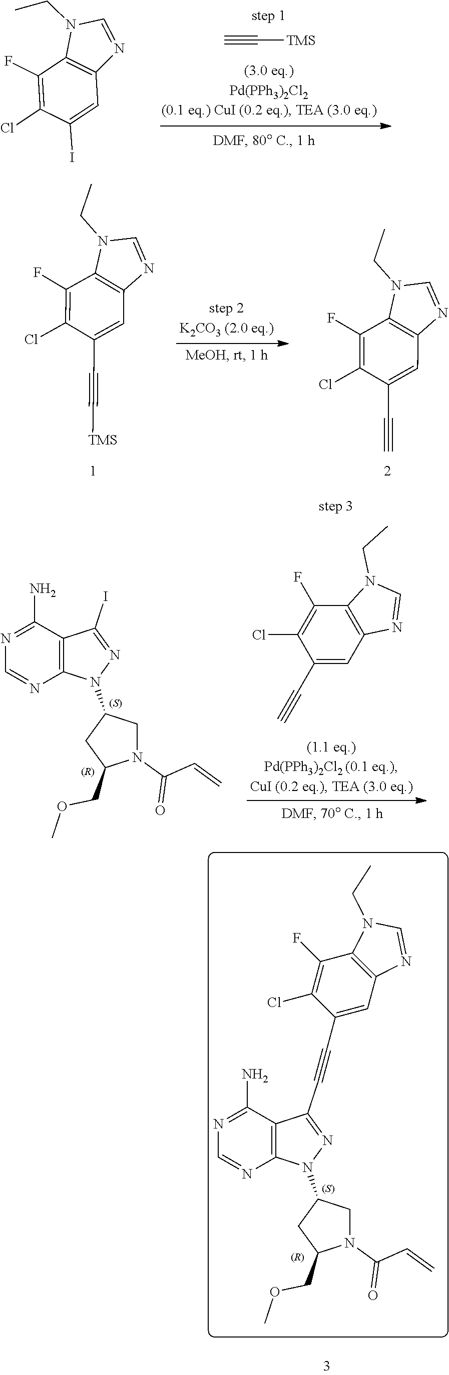

1-[(2R,4S)-4-[4-amino-3-[2-(6-chloro-1-ethyl-7-fluoro-1,3-benzodiazol-5-yl)ethynyl]pyrazolo[3,4-d]pyrimidin-1-yl]-2-(methoxymethyl)pyrrolidin-1-yl]prop-2-en-1-one. MS ESI calculated for C₂₅H₂₄ClFN₈O₂ [M+H]⁺, 523.17, found 523.10; ¹H NMR (400 MHz, DMSO-d₆) δ 8.45 (s, 1H), 8.31 (s, 1H), 8.10 (s, 1H), 6.65 (m, 1H), 6.23-6.10 (m, 1H), 5.78-5.57 (m, 2H), 4.53-4.45 (m, 1H), 4.39-4.31 (m, 2H), 4.12-4.05 (m, 1H), 4.03-3.91 (m, 1H), 3.81 (m, 1H), 3.70-3.58 (m, 1H), 3.48 (d, J=4.8 Hz, 1H), 3.32 (d, J=5.3 Hz, 3H), 2.73-2.54 (m, 1H), 2.34 (t, J=2.0 Hz, 1H), 1.45 (t, J=7.2 Hz, 3H).

### Example 129: 1-[(2R,4S)-4-(4-Amino-3-{2-[6-chloro-1-(difluoromethyl)-2-methyl-1,3-benzodiazol-5-yl]ethynyl}pyrazolo[3,4-d]pyrimidin-1-yl)-2-(methoxymethyl)pyrrolidin-1-yl]prop-2-en-1-one

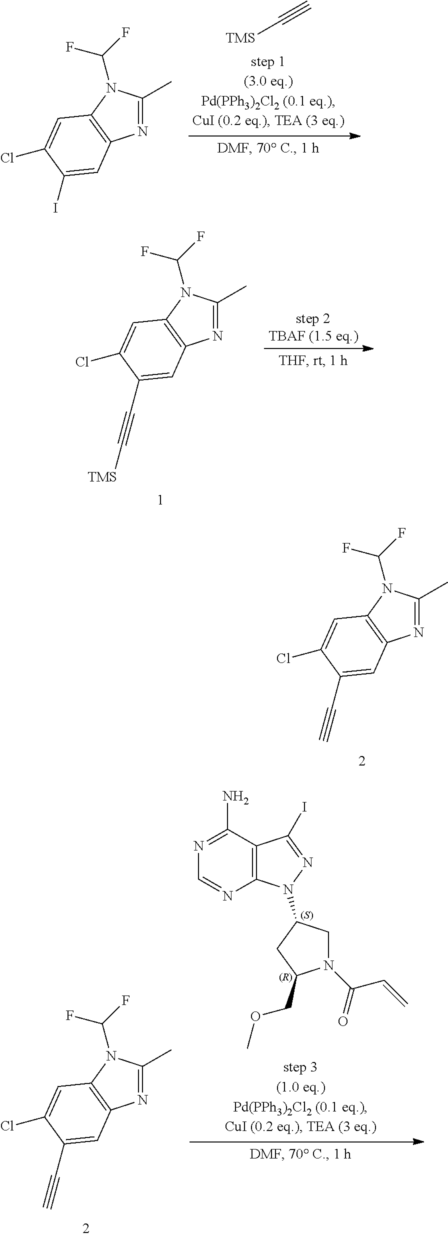

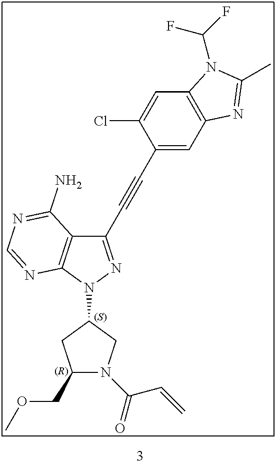

1-[(2R,4S)-4-(4-amino-3-{2-[6-chloro-1-(difluoromethyl)-2-methyl-1,3-benzodiazol-5-yl]ethynyl}pyrazolo[3,4-d]pyrimidin-1-yl)-2-(methoxymethyl)pyrrolidin-1-yl]prop-2-en-1-one. MS ESI calculated for C₂₅H₂₃ClF₂N₈O₂ [M+H]⁺, 540.96, found 540.16; ¹H NMR (400 MHz, CDCl₃) δ 8.41-8.39 (m, 1H), 8.03-8.02 (m, 1H), 7.73 (s, 1H), 7.45-7.30 (m, 1H), 6.60-6.34 (m, 4H), 5.89-5.85 (m, 1H), 5.76-5.70 (m, 1H), 4.7-4.53 (m, 1H), 4.19-4.04 (m, 2H), 3.86-3.83 (m, 1H), 3.57-3.52 (m, 1H), 3.42 (s, 3H), 2.86-2.78 (m, 1H), 2.75 (s, 3H), 2.53-2.49 (m, 1H).

### Example 130: 1-[(2R,4S)-4-(4-Amino-3-{2-[6-chloro-1-(difluoromethyl)-2-methyl-1,3-benzodiazol-5-yl]ethynyl}pyrazolo[4,3-c]pyridin-1-yl)-2-(methoxymethyl)pyrrolidin-1-yl]prop-2-en-1-one

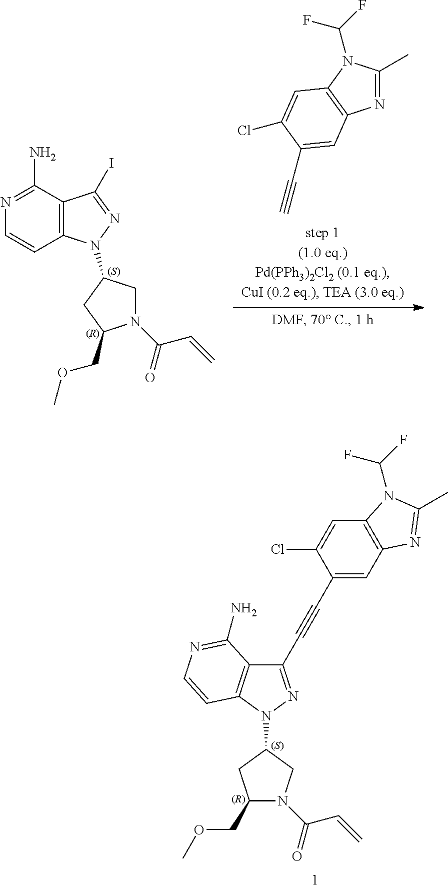

1-[(2R,4S)-4-(4-amino-3-{2-[6-chloro-1-(difluoromethyl)-2-methyl-1,3-benzodiazol-5-yl]ethynyl}pyrazolo[4,3-c]pyridin-1-yl)-2-(methoxymethyl)pyrrolidin-1-yl]prop-2-en-1-one. MS ESI calculated for C₂₆H₂₄ClF₂N₇O₂ [M+H]⁺, 540.16, found 540.25; ¹H NMR (400 MHz, CDCl₃) δ 8.03 (d, J=4 Hz, 1H), 7.90 (d, J=8 Hz, 1H), 7.72 (s, 1H), 7.49 (s, 1H), 6.75 (d, J=8 Hz, 1H), 6.57-6.43 (m, 2H), 5.87-5.71 (m, 3H), 5.56-5.35 (m, 1H), 4.68-4.52 (m, 1H), 4.19-3.91 (m, 3H), 3.57-3.43 (m, 4H), 2.83-2.75 (m, 4H), 2.49-2.42 (m, 1H).

### Example 131: 1-[(2R,4S)-4-(4-Amino-5-{2-[6-chloro-1-(difluoromethyl)-2-methyl-1,3-benzodiazol-5-yl]ethynyl}pyrrolo[2,3-d]pyrimidin-7-yl)-2-(methoxymethyl)pyrrolidin-1-yl]prop-2-en-1-one

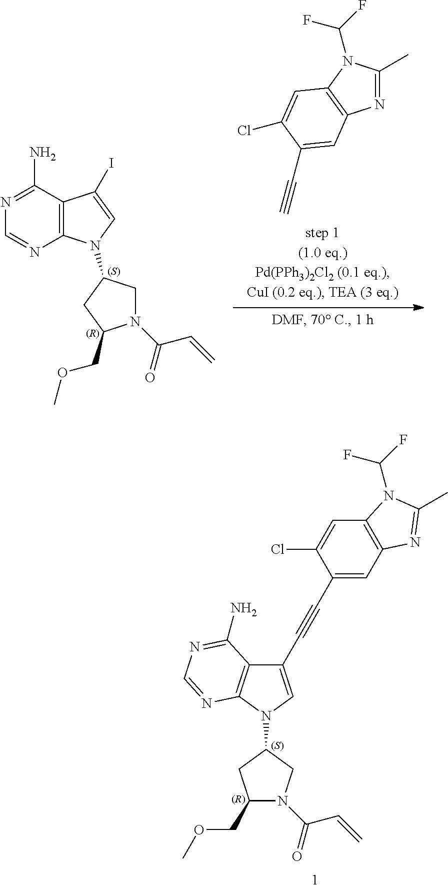

1-[(2R,4S)-4-(4-amino-5-{2-[6-chloro-1-(difluoromethyl)-2-methyl-1,3-benzodiazol-5-yl]ethynyl}pyrrolo[2,3-d]pyrimidin-7-yl)-2-(methoxymethyl)pyrrolidin-1-yl]prop-2-en-1-one. MS ESI calculated for C₂₆H₂₄ClF₂N₇O₂ [M+H]⁺, 540.16, found 540.25; ¹H NMR (400 MHz, CDCl₃) δ 8.30 (d, J=12 Hz, 1H), 7.87 (d, J=4 Hz, 1H), 7.67 (s, 1H), 7.43-7.33 (m, 1H), 6.57-6.39 (m, 1H), 6.20 (s, 2H), 5.79-5.57 (m, 2H), 4.68-4.21 (m, 1H), 4.21-4.19 (m, 1H), 4.10-4.06 (s, 1H), 3.84-3.80 (m, 1H), 3.57-3.52 (m, 1H), 3.42 (s, 3H), 2.72 (s, 3H), 2.58-2.51 (m, 2H).

### Example 132: 1-[(3S)-3-{4-Amino-3-[2-(6-chloro-1-ethyl-1,3-benzodiazol-5-yl)ethynyl]pyrazolo[3,4-d]pyrimidin-1-yl}pyrrolidin-1-yl]prop-2-en-1-one

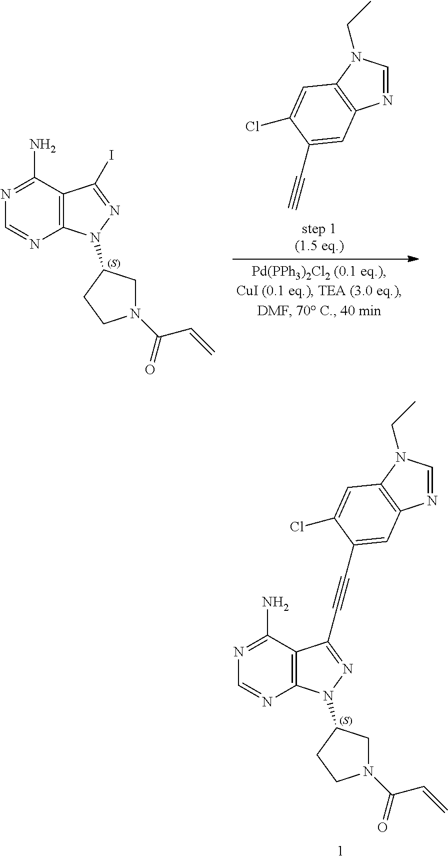

1-[(3S)-3-{4-amino-3-[2-(6-chloro-1-ethyl-1,3-benzodiazol-5-yl)ethynyl]pyrazolo[3,4-d]pyrimidin-1-yl}pyrrolidin-1-yl]prop-2-en-1-one. MS ESI calculated for C₂₃H₂₁ClN₈O [M+H]⁺, 461.15, found 461.10; ¹H NMR (300 MHz, DMSO-d₆) δ 8.57-7.87 (m, 5H), 6.62-6.32 (m, 1H), 6.17-5.86 (m, 1H), 5.69-5.56 (m, 1H), 5.51-5.21 (m, 1H), 4.39-4.32 (m, 2H), 4.18-3.52 (m, 4H), 2.47-2.32 (m, 2H), 1.41-1.20 (m, 3H).

### Example 133: (S)-1-(3-(4-Amino-3-((6-chloro-1-ethyl-1H-benzo[d]imidazol-5-yl)ethynyl)-1H-pyrazolo[4,3-c]pyridin-1-yl)pyrrolidin-1-yl)prop-2-en-1-one

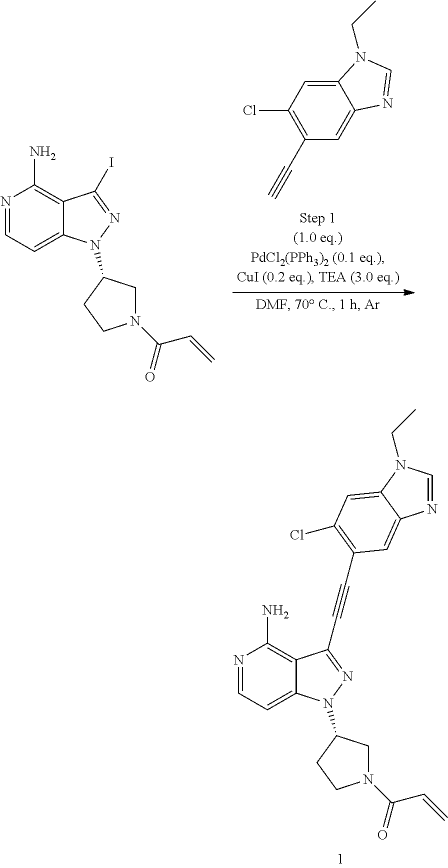 [SMILES: C=CC(=O)N1CC[C@H](n2nc(I)c3c(N)nccc32)C1 ; C#Cc1cc2ncn(CC)c2cc1Cl ; C=CC(=O)N1CC[C@H](n2nc(C#Cc3cc4ncn(CC)c4cc3Cl)c3c(N)nccc32)C1]

(S)-1-(3-(4-amino-3-((6-chloro-1-ethyl-1H-benzo[d]imidazol-5-yl)ethynyl)-1H-pyrazolo[4,3-c]pyridin-1-yl)pyrrolidin-1-yl)prop-2-en-1-one. ESI calculated for C₂₄H₂₂ClN₇O [M+H]⁺, 460.16, found 460.25; ¹H NMR (400 MHz, DMSO-d₆) δ 8.45 (s, 1H), 8.13 (s, 1H), 8.04 (s, 1H), 7.81 (dd, J=6.1, 1.2 Hz, 1H), 6.97 (dd, J=7.2, 6.1 Hz, 1H), 6.74-6.46 (m, 3H), 6.13-6.27 (m, 1H), 5.63-5.78 (m, 1H), 5.53-5.34 (m, 1H), 4.27-4.39 (m, 2H), 4.19-3.55 (m, 4H), 2.30-2.47 (m, 2H), 1.36-1.48 (m, 3H).

### Example 134: 1-[(2R,4S)-4-[4-Amino-3-[2-(4,6-difluoro-1-methyl-1,3-benzodiazol-5-yl)ethynyl]pyrazolo[3,4-d]pyrimidin-1-yl]-2-[(2H3)methoxymethyl]pyrrolidin-1-yl]prop-2-en-1-one

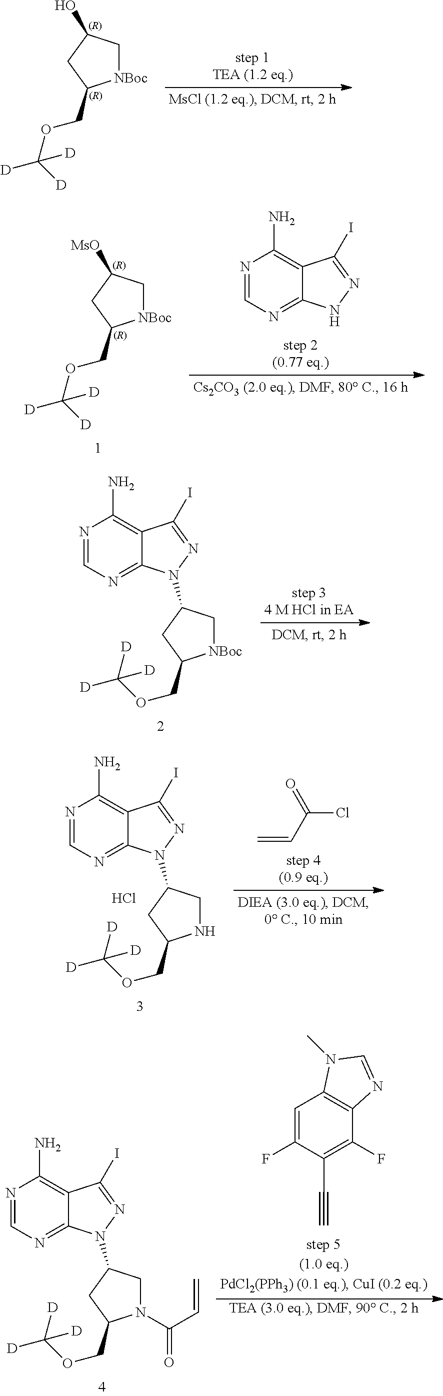

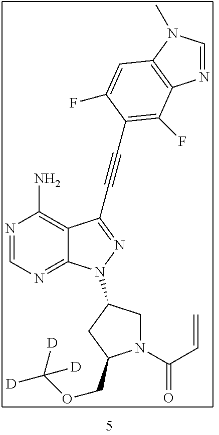 [SMILES: [2H]C([2H])([2H])OC[C@H]1C[C@H](n2nc(C#Cc3c(F)cc4c(ncn4C)c3F)c3c(N)ncnc32)CN1C(=O)C=C]

1-[(2R,4S)-4-[4-amino-3-[2-(4,6-difluoro-1-methyl-1,3-benzodiazol-5-yl)ethynyl]pyrazolo[3,4-d]pyrimidin-1-yl]-2-[(2H³)methoxymethyl]pyrrolidin-1-yl]prop-2-en-1-one. MS ESI calculated for C₂₄H₁₉D₃F₂N₈O₂ [M+H]⁺, 496.20, found 496.20; ¹H NMR (400 MHz, CDCl₃) δ 8.41 (s, 1H), 7.94 (s, 1H), 7.10-7.04 (m, 1H), 6.49-6.37 (m, 2H), 6.25 (brs, 2H), 5.92-5.70 (m, 2H), 4.74-4.65 (m, 1H), 4.21-4.05 (m, 2H), 3.89-3.82 (m, 4H), 3.55-3.49 (m, 1H), 2.89-2.76 (m, 1H), 2.50-2.48 (m, 1H).

### Example 135: 1-[(2R,4S)-4-[4-Amino-3-[2-(6-chloro-1-ethyl-1,3-benzodiazol-5-yl) ethynyl]pyrazolo[3,4-d]pyrimidin-1-yl]-2-[(2H3)methoxymethyl]pyrrolidin-1-yl]prop-2-en-1-one

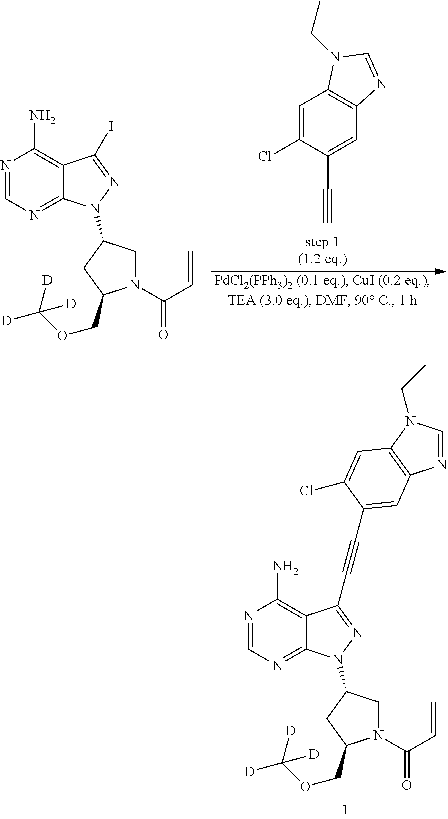 [SMILES: [2H]C([2H])([2H])OC[C@H]1C[C@H](n2nc(I)c3c(N)ncnc32)CN1C(=O)C=C ; C#Cc1cc2ncn(CC)c2cc1Cl ; "PdCl2(PPh3)2 (0.1 eq.), CuI (0.2 eq.),TEA (3.0 eq.), DMF, 90° C., 1 h" ; [2H]C([2H])([2H])OC[C@H]1C[C@H](n2nc(C#Cc3cc4ncn(CC)c4cc3Cl)c3c(N)ncnc32)CN1C(=O)C=C]

1-[(2R,4S)-4-[4-amino-3-[2-(6-chloro-1-ethyl-1,3-benzodiazol-5-yl)ethynyl]pyrazolo[3,4-d]pyrimidin-1-yl]-2-[(2H³)methoxymethyl]pyrrolidin-1-yl]prop-2-en-1-one. MS ESI calculated for C₂₅H₂₂D₃ClN₈O₂ [M+H]⁺, 508.20, found 508.25; ¹H NMR (300 MHz, DMSO-d₆) δ 8.46 (s, 1H), 8.31 (d, J=1.8 Hz, 1H), 8.21 (s, 1H), 8.04 (s, 1H), 6.81-6.55 (m, 2H), 6.21-6.16 (m, 1H), 5.81-5.52 (m, 2H), 4.61-4.48 (m, 1H), 4.34-4.29 (m, 2H), 4.17-3.77 (m, 2H), 3.69-3.43 (m, 2H), 2.76-2.57 (m, 1H), 2.46-2.31 (m, 1H), 1.43 (t, J=7.2 Hz, 3H).

### Example 136: (S)-1-(3-(4-Amino-3-((6-chloro-1-ethyl-7-fluoro-1H-benzo[d]imidazol-5-yl)ethynyl)-1H-pyrazolo[4,3-c]pyridin-1-yl)pyrrolidin-1-yl)prop-2-en-1-one

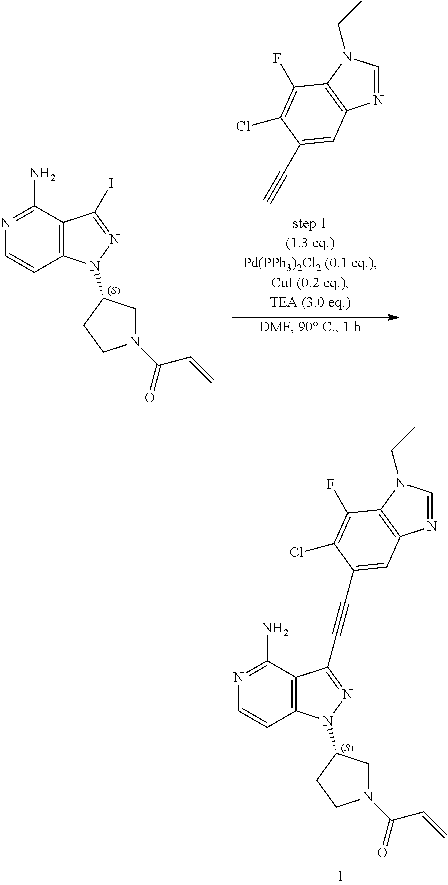

(S)-1-(3-(4-amino-3-((6-chloro-1-ethyl-7-fluoro-1H-benzo[d]imidazol-5-yl)ethynyl)-1H-pyrazolo[4,3-c]pyridin-1-yl)pyrrolidin-1-yl)prop-2-en-1-one. MS ESI calculated for C₂₄H₂₁ClFN₇O [M+H]⁺, 478.15, found 478.10; ¹H NMR (400 MHz, DMSO-d₆) δ 8.47 (s, 1H), 8.05 (s, 1H), 7.81 (d, J=6.1 Hz, 1H), 6.98 (t, J=6.6 Hz, 1H), 6.67-6.63 (m, 1H), 6.51 (s, 2H), 6.20-6.17 (m, 1H), 5.78-5.70 (m, 1H), 5.49-5.44 (m, 1H), 4.47-4.40 (m, 2H), 4.17-3.84 (m, 2H), 3.82-3.53 (m, 2H), 2.46-2.41 (m, 2H), 1.46 (t, J=7.2 Hz, 3H)

### Example 137: (S)-1-(3-(4-Amino-3-((6-chloro-1-methyl-1H-benzo[d]imidazol-5-yl)ethynyl)-1H-pyrazolo[4,3-c]pyridin-1-yl)pyrrolidin-1-yl)prop-2-en-1-one

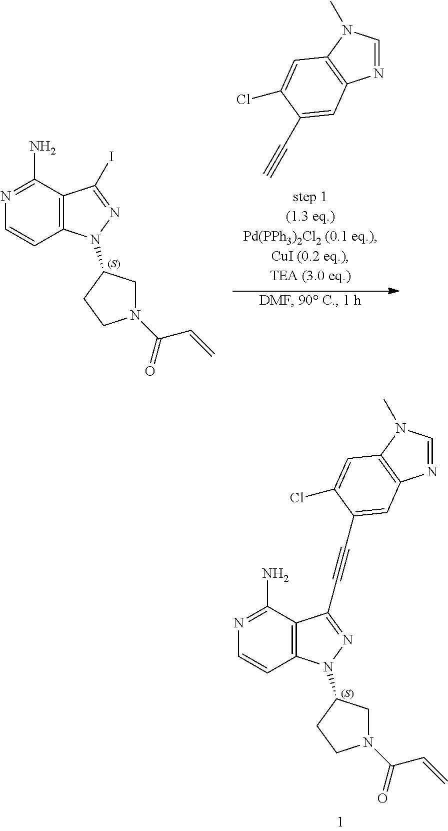

(S)-1-(3-(4-amino-3-((6-chloro-1-methyl-1H-benzo[d]imidazol-5-yl)ethynyl)-1H-pyrazolo[4,3-c]pyridin-1-yl)pyrrolidin-1-yl)prop-2-en-1-one. MS ESI calculated for C₂₃H₂₀ClN₇O [M+H]⁺, 446.14, found 446.20; ¹H NMR (400 MHz, DMSO-d₆) δ 8.36 (s, 1H), 8.12 (s, 1H), 7.98 (s, 1H), 7.81 (d, J=6.1 Hz, 1H), 6.97 (dd, J=7.1, 6.2 Hz, 1H), 6.76-6.38 (m, 3H), 6.20-6.17 (m, 1H), 5.79-5.70 (m, 1H), 5.44-5.34 (m, 1H), 4.19-3.88 (m, 2H), 3.79 (d, J=4.7 Hz, 2H), 3.30 (s, 3H), 2.46-2.42 (m, 2H).

### Example 138: 1-[(2R,4S)-4-{4-Amino-3-[2-(6-chloro-1-cyclopropyl-1,3-benzodiazol-5-yl)ethynyl]pyrazolo[4,3-c]pyridin-1-yl}-2-(methoxymethyl)pyrrolidin-1-yl]prop-2-en-1-one

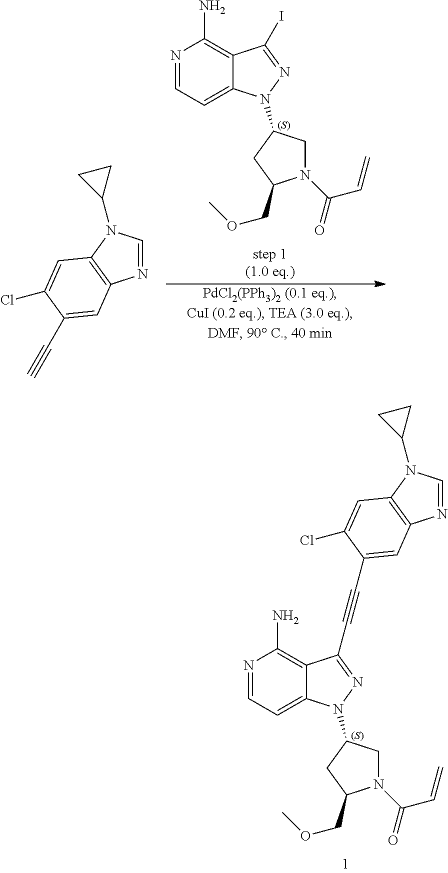

1-[(2R,4S)-4-{4-amino-3-[2-(6-chloro-1-cyclopropyl-1,3-benzodiazol-5-yl)ethynyl]pyrazolo[4,3-c]pyridin-1-yl}-2-(methoxymethyl)pyrrolidin-1-yl]prop-2-en-1-one. MS ESI calculated for C₂₇H₂₆ClN₇O₂ [M+H]⁺, 516.18, 518.18, found 516.05, 518.05; ¹H NMR (400 MHz, CDCl₃) δ 8.11 (d, J=5.5 Hz, 1H), 8.01 (s, 1H), 7.79-7.70 (m, 1H), 7.71 (s, 1H), 6.78 (d, J=6.4 Hz, 1H), 6.64-6.39 (m, 2H), 5.79-5.69 (m, 1H), 5.59-5.31 (m, 1H), 4.70-4.48 (m, 1H), 4.18-4.02 (m, 2H), 3.95-3.92 (m, 1H), 3.60-3.38 (m, 5H), 3.02-2.72 (m, 1H), 2.59-2.41 (m, 1H), 1.31-1.20 (m, 2H), 1.13-1.06 (m, 2H).

### Example 139: 1-[(2R,4S)-4-{4-Amino-3-[2-(4,6-difluoro-1-methyl-1,3-benzodiazol-5-yl)ethynyl]pyrazolo[3,4-d]pyrimidin-1-yl}-2-[(difluoromethoxy)methyl]pyrrolidin-1-yl]prop-2-en-1-one

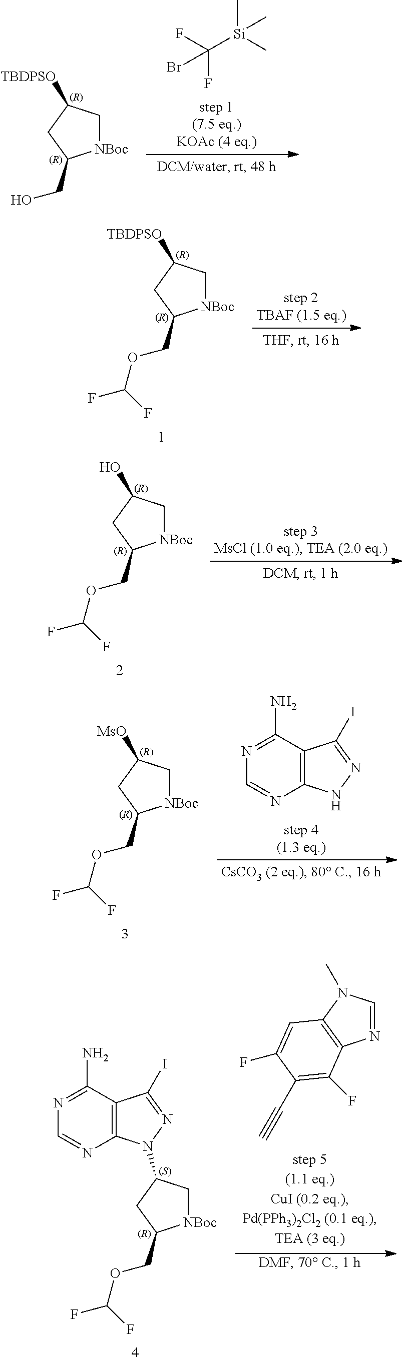

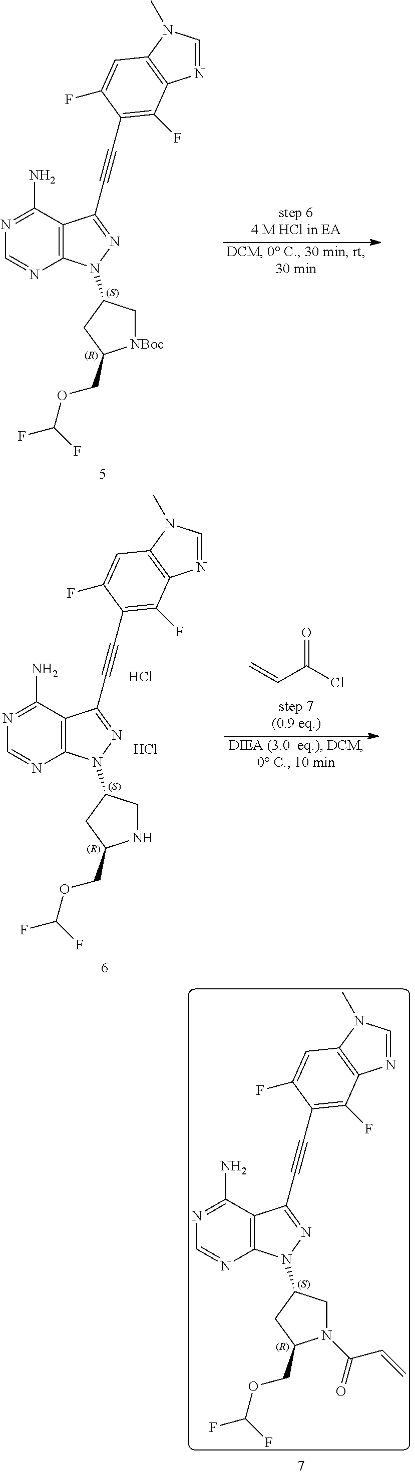

1-[(2R,4S)-4-{4-amino-3-[2-(4,6-difluoro-1-methyl-1,3-benzodiazol-5-yl)ethynyl]pyrazolo[3,4-d]pyrimidin-1-yl}-2-[(difluoromethoxy)methyl]pyrrolidin-1-yl]prop-2-en-1-one. MS ESI calculated for C₂₄H₂₀F₄N₈O₂ [M+H]⁺, 529.16, found 529.15; ¹H NMR (300 MHz, DMSO-d₆) δ 8.37 (s, 1H), 8.29 (d, J=1.6 Hz, 1H), 7.64 (d, J=9.1 Hz, 1H), 7.04-6.67 (m, 1H), 6.62-6.45 (m, 1H), 6.16-6.11 (m, 1H), 5.7-5.52 (m, 2H), 4.63-4.45 (m, 1H), 4.25-4.09 (m, 1H), 4.09-3.90 (m, 3H), 3.85 (s, 3H), 2.86-2.57 (m, 1H), 2.45-2.31 (m, 1H).

### Example 140: 1-[(3S)-3-{4-Amino-3-[2-(6-fluoro-1-methyl-1,3-benzodiazol-5-yl)ethynyl]pyrazolo[3,4-d]pyrimidin-1-yl}pyrrolidin-1-yl]prop-2-en-1-one

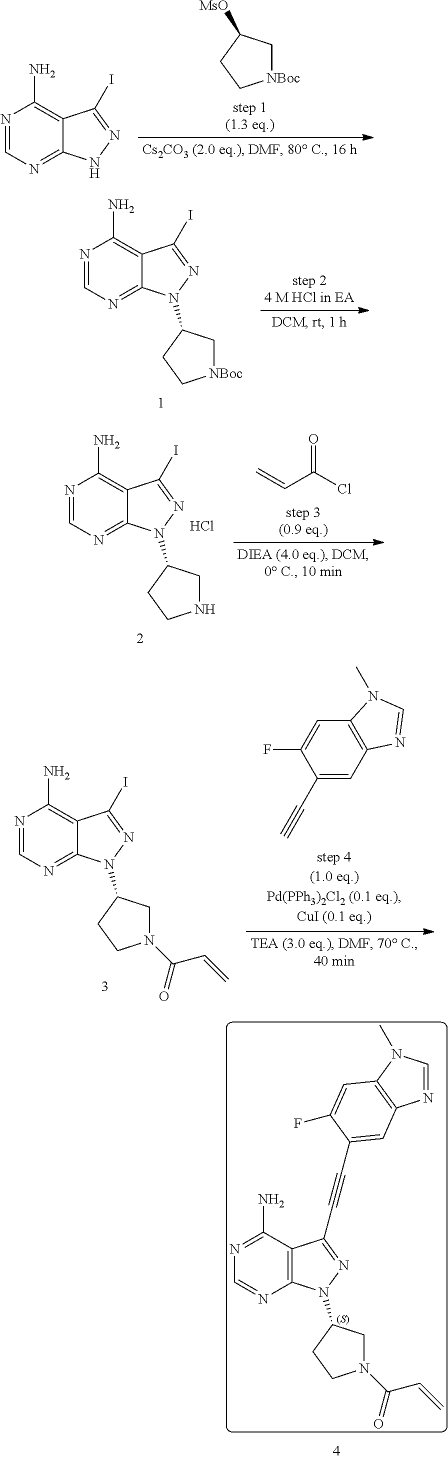

1-[(3S)-3-{4-amino-3-[2-(6-fluoro-1-methyl-1,3-benzodiazol-5-yl)ethynyl]pyrazolo[3,4-d]pyrimidin-1-yl}pyrrolidin-1-yl]prop-2-en-1-one. MS ESI calculated for C₂₂H₁₉FN₈O [M+H]⁺, 431.17, found 431.10; ¹H NMR (300 MHz, DMSO-d₆) δ 8.33 (d, J=9.5 Hz, 2H), 8.16 (dd, J=6.3, 2.0 Hz, 1H), 7.72 (d, J=9.8 Hz, 1H), 6.65-6.56 (m, 1H), 6.19-6.15 (m, 1H), 5.72-5.67 (m, 1H), 5.63-5.41 (m, 1H), 4.24-3.78 (m, 6H), 3.77-3.55 (m, 1H), 2.41-2.30 (m, 2H).

### Example 141: 1-[(3S)-3-{4-Amino-3-[2-(6-fluoro-1-methyl-1,3-benzodiazol-5-yl)ethynyl]pyrazolo[4,3-c]pyridin-1-yl}pyrrolidin-1-yl]prop-2-en-1-one

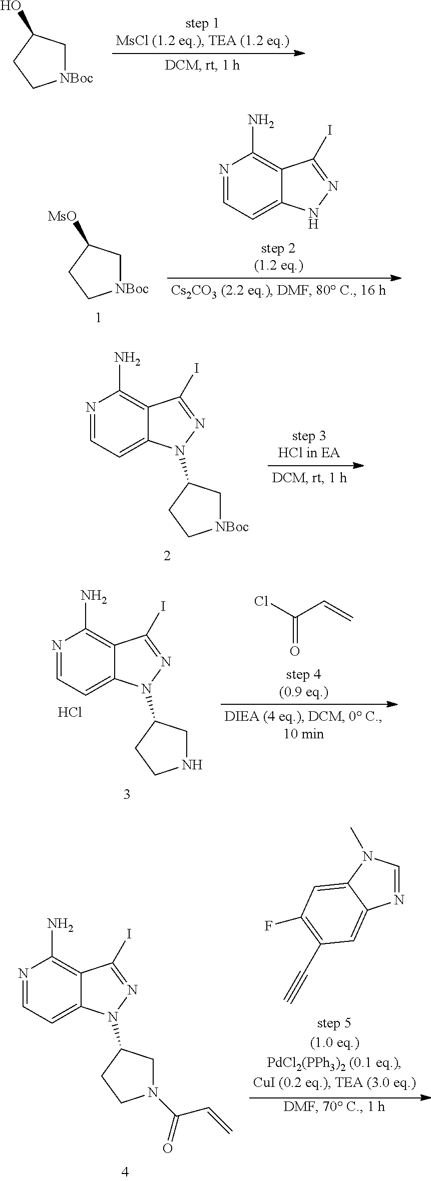

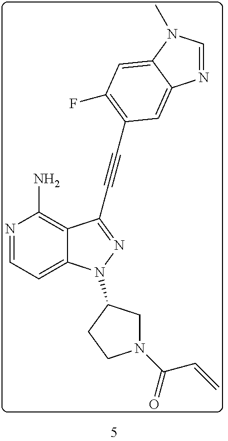 [SMILES: C=CC(=O)N1CC[C@H](n2nc(C#Cc3cc4ncn(C)c4cc3F)c3c(N)nccc32)C1]

1-[(3S)-3-{4-amino-3-[2-(6-fluoro-1-methyl-1,3-benzodiazol-5-yl)ethynyl]pyrazolo[4,3-c]pyridin-1-yl}pyrrolidin-1-yl]prop-2-en-1-one. MS ESI calculated for C₂₃H₂₀FN₇O [M+H]⁺, 430.17, found 430.05; ¹H NMR (400 MHz, DMSO-d₆) δ 8.34 (s, 1H), 8.07 (dd, J=6.4, 2.1 Hz, 1H), 7.77 (dd, J=35.8, 7.9 Hz, 2H), 6.97-6.92 (m, 1H), 6.78-6.40 (m, 3H), 6.22-6.13 (m, 1H), 5.73-5.68 (m, 1H), 5.56-5.33 (m, 1H), 4.14-4.01 (m, 1H), 3.98-3.55 (m, 6H), 2.48-2.25 (m, 2H).

### Example 142: (S)-1-(3-(4-Amino-5-((6-fluoro-1-methyl-1H-benzo[d]imidazol-5-yl)ethynyl)-7H-pyrrolo[2,3-d]pyrimidin-7-yl)pyrrolidin-1-yl)prop-2-en-1-one

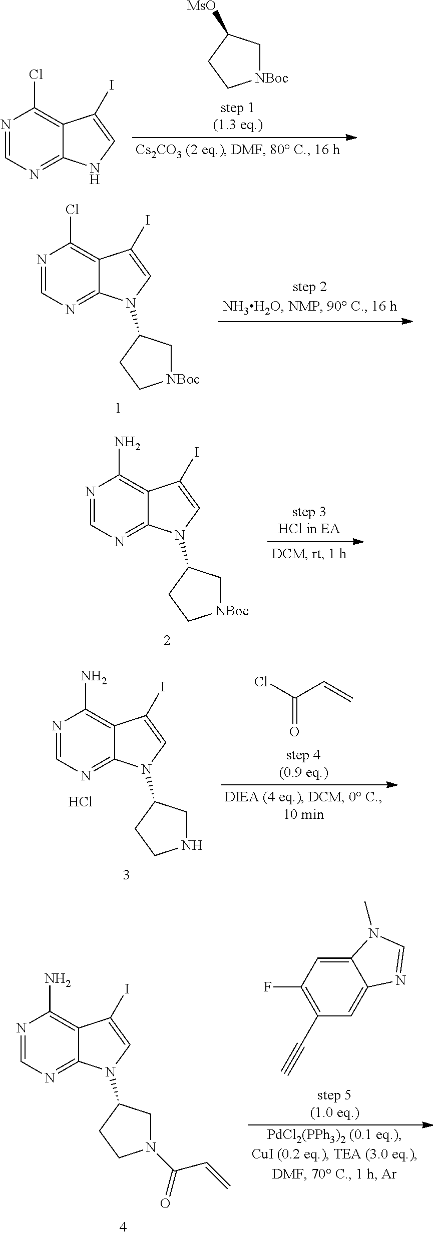

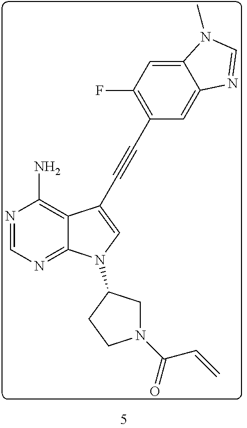 [SMILES: C=CC(=O)N1CC[C@H](n2cc(C#Cc3cc4ncn(C)c4cc3F)c3c(N)ncnc32)C1]

(S)-1-(3-(4-amino-5-((6-fluoro-1-methyl-1H-benzo[d]imidazol-5-yl)ethynyl)-7H-pyrrolo[2,3-d]pyrimidin-7-yl)pyrrolidin-1-yl)prop-2-en-1-one. MS ESI calculated for C₂₃H₂₀FN₇O [M+H]⁺, 430.17, found 430.15; ¹H NMR (400 MHz, DMSO-d₆) δ 8.29 (s, 1H), 8.19 (d, J=1.3 Hz, 1H), 7.92 (dd, J=6.4, 3.4 Hz, 1H), 7.77 (d, J=16.6 Hz, 1H), 7.66 (d, J=9.8 Hz, 1H), 6.53-6.72 (m, 2H), 6.13-6.23 (m, 1H), 5.65-5.77 (m, 1H), 5.24-5.41 (m, 1H), 4.18-3.86 (m, 2H), 3.84 (s, 3H), 3.80-3.66 (m, 2H), 3.48-3.59 (m, 1H), 2.49-2.30 (m, 2H).

### Example 143: (S)-1-(3-(4-Amino-5-((6-chloro-1-ethyl-1H-benzo[d]imidazol-5-yl)ethynyl)-7H-pyrrolo[2,3-d]pyrimidin-7-yl)pyrrolidin-1-yl)prop-2-en-1-one

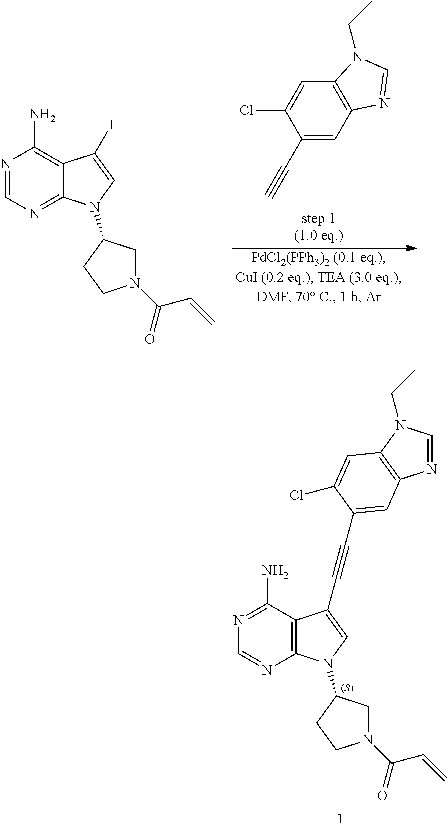

(S)-1-(3-(4-amino-5-((6-chloro-1-ethyl-1H-benzo[d]imidazol-5-yl)ethynyl)-7H-pyrrolo[2,3-d]pyrimidin-7-yl)pyrrolidin-1-yl)prop-2-en-1-one. MS ESI calculated for C₂₄H₂₂ClN₇O [M+H]⁺, 460.16, found 460.25; ¹H NMR (400 MHz, DMSO-d₆) δ 8.45 (s, 1H), 8.13 (s, 1H), 8.04 (s, 1H), 7.81 (dd, J=6.1, 1.2 Hz, 1H), 6.97 (dd, J=7.2, 6.1 Hz, 1H), 6.74-6.46 (m, 3H), 6.13-6.27 (m, 1H), 5.63-5.78 (m, 1H), 5.53-5.34 (m, 1H), 4.27-4.39 (m, 2H), 4.19-3.55 (m, 4H), 2.30-2.47 (m, 2H), 1.36-1.48 (m, 3H).

### Example 144: 1-[(3S)-3-{4-Amino-3-[2-(6-chloro-1-ethyl-7-fluoro-1,3-benzodiazol-5-yl)ethynyl]pyrazolo[3,4-d]pyrimidin-1-yl}pyrrolidin-1-yl]prop-2-en-1-one

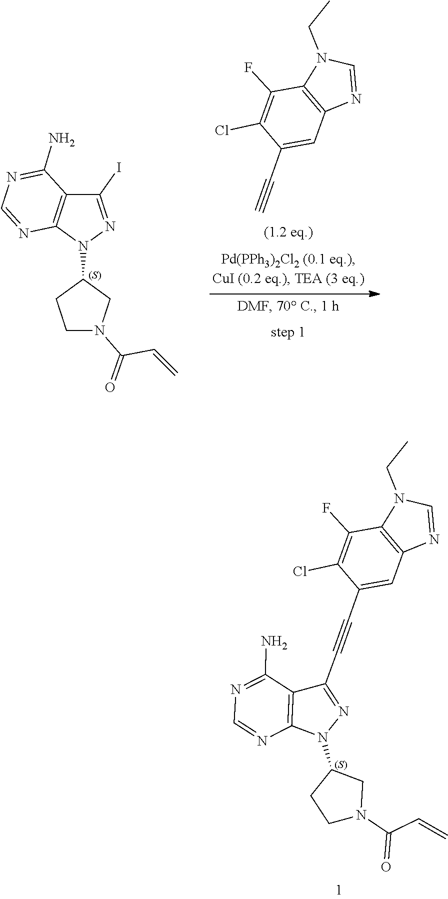

1-[(3S)-3-{4-amino-3-[2-(6-chloro-1-ethyl-7-fluoro-1,3-benzodiazol-5-yl)ethynyl]pyrazolo[3,4-d]pyrimidin-1-yl}pyrrolidin-1-yl]prop-2-en-1-one. MS ESI calculated for C₂₃H₂₀ClFN₈O [M+H]⁺, 479.14, found 479.25; ¹H NMR (400 MHz, CDCl₃) δ 8.38 (d, J=4 Hz, 1H), 7.97 (d, J=4 Hz, 2H), 6.57-6.42 (m, 2H), 6.17 (s, 2H), 5.75-5.71 (m, 1H), 5.69-5.53 (m, 1H), 4.42 (q, J=48 Hz, 2H), 4.15-4.01 (m, 3H), 3.82-3.74 (m, 1H), 2.71-2.63 (m, 1H), 2.56-2.45 (m, 1H), 1.60 (t, J=16 Hz, 3H).

### Example 145: (S)-1-(3-(4-Amino-3-((6-chloro-7-fluoro-1-methyl-1H-benzo[d]imidazol-5-yl)ethynyl)-1H-pyrazolo[3,4-d]pyrimidin-1-yl)pyrrolidin-1-yl)prop-2-en-1-one

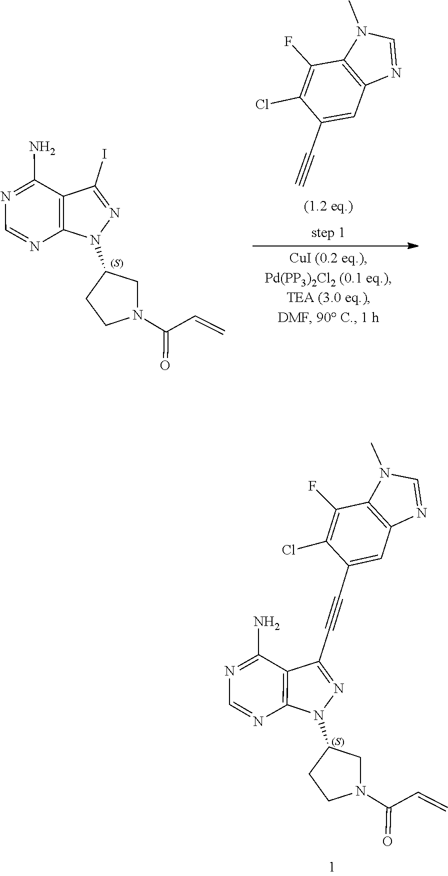

(S)-1-(3-(4-amino-3-((6-chloro-7-fluoro-1-methyl-1H-benzo[d]imidazol-5-yl)ethynyl)-1H-pyrazolo[3,4-d]pyrimidin-1-yl)pyrrolidin-1-yl)prop-2-en-1-one. MS ESI calculated for C₂₂H₁₈ClFN₈O [M+H]⁺, 465.13, found 465.25; ¹H NMR (300 MHz, DMSO-d₆) δ 8.38 (s, 1H), 8.30 (d, J=0.9 Hz, 1H), 8.12 (s, 1H), 6.63-6.61 (m, 1H), 6.17-6.04 (m, 1H), 5.86-5.39 (m, 2H), 4.35-3.40 (m, 7H), 2.38-2.31 (m, 2H).

### Example 146: (S)-1-(3-(4-Amino-3-((1-cyclopropyl-6-fluoro-1H-benzo[d]imidazol-5-yl)ethynyl)-1H-pyrazolo[3,4-d]pyrimidin-1-yl)pyrrolidin-1-yl)prop-2-en-1-one

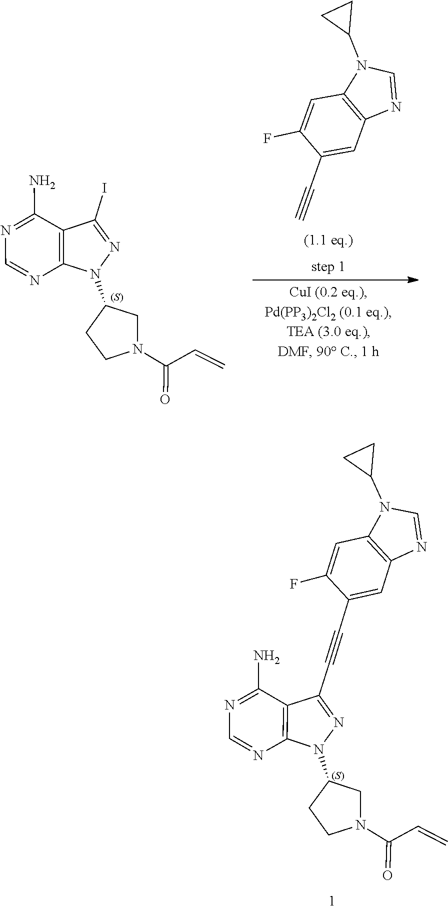

(S)-1-(3-(4-amino-3-((1-cyclopropyl-6-fluoro-1H-benzo[d]imidazol-5-yl)ethynyl)-1H-pyrazolo[3,4-d]pyrimidin-1-yl)pyrrolidin-1-yl)prop-2-en-1-one. MS ESI calculated for C₂₄H₂₁FN₈O [M+H]⁺, 457.18, found 457.25; ¹H NMR (300 MHz, DMSO-d₆) δ 8.38 (s, 1H), 8.30 (s, 1H), 8.16 (dd, J=6.3, 2.0 Hz, 1H), 7.66 (d, J=9.5 Hz, 1H), 6.63-6.58 (m, 1H), 6.19-6.13 (m, 1H), 5.78-5.35 (m, 2H), 4.03-3.77 (m, 2H), 3.76-3.46 (m, 2H), 2.54 (s, 1H), 2.40-2.36 (m, 2H), 1.09-1.03 (m, 4H).

### Example 147: 1-[(3S)-3-{4-Amino-3-[2-(6-chloro-1-methyl-1,3-benzodiazol-5-yl)ethynyl]pyrazolo[3,4-d]pyrimidin-1-yl}pyrrolidin-1-yl]prop-2-en-1-one

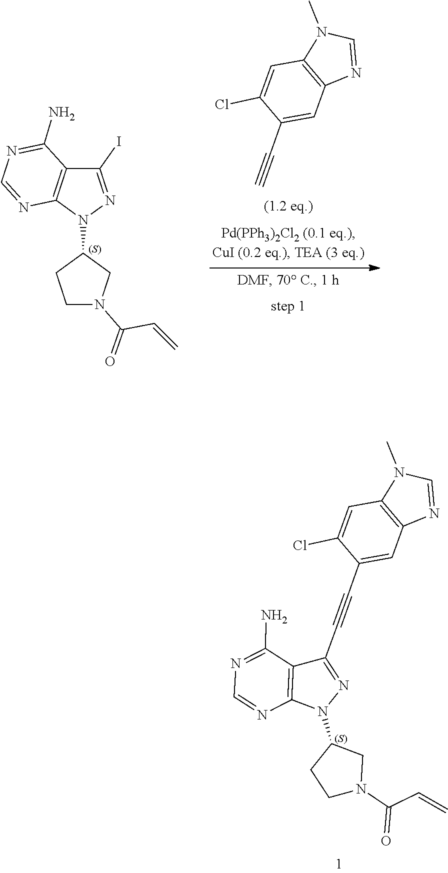

1-[(3S)-3-{4-amino-3-[2-(6-chloro-1-methyl-1,3-benzodiazol-5-yl)ethynyl]pyrazolo[3,4-d]pyrimidin-1-yl}pyrrolidin-1-yl]prop-2-en-1-one. MS ESI calculated for C₂₂H₁₉ClN₈O [M+H]⁺, 447.14, found 447.20; ¹H NMR (400 MHz, CDCl₃) δ 8.37 (d, J=4 Hz, 1H), 8.14 (s, 1H), 7.95 (s, 1H), 7.53 (s, 1H), 6.57-6.40 (m, 2H), 6.40-5.75 (m, 2H), 5.75-5.68 (m, 1H), 5.68-5.53 (m, 1H), 4.15-4.00 (m, 3H), 3.87 (s, 3H), 3.83-3.73 (m, 1H), 2.74-2.63 (m, 1H), 2.54-2.45 (m, 1H).

### Example 148: 1-[(3S)-3-{4-Amino-3-[2-(6-chloro-7-fluoro-1-methyl-1,3-benzodiazol-5-yl)ethynyl]pyrazolo[4,3-c]pyridin-1-yl}pyrrolidin-1-yl]prop-2-en-1-one

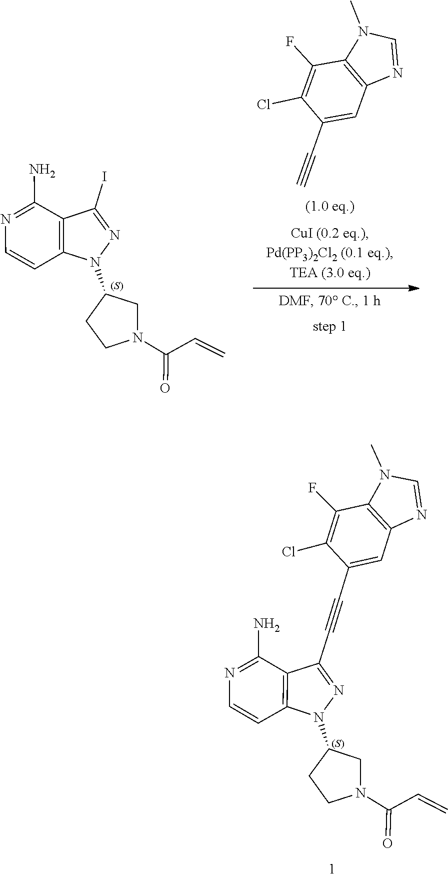

1-[(3S)-3-{4-amino-3-[2-(6-chloro-7-fluoro-1-methyl-1,3-benzodiazol-5-yl)ethynyl]pyrazolo[4,3-c]pyridin-1-yl}pyrrolidin-1-yl]prop-2-en-1-one. MS ESI calculated for C₂₃H₁₉ClFN₇O [M+H]⁺464.13, found 464.10; ¹H NMR (300 MHz, DMSO-d₆) δ 8.39 (s, 1H), 8.03 (s, 1H), 7.83 (m, 1H), 6.99 (m, 1H), 6.75-6.50 (m, 3H), 6.23-6.11 (m, 1H), 5.71 (m, 1H), 5.45 (m, 1H), 4.16 (m, 1H), 4.02 (d, J=1.4 Hz, 3H), 3.93-3.86 (m, 1H), 3.82-3.47 (m, 2H), 2.49-2.26 (m, 2H).

### Example 149: 1-[(3S)-3-{4-Amino-3-[2-(1-cyclopropyl-6-fluoro-1,3-benzodiazol-5-yl)ethynyl]pyrazolo[4,3-c]pyridin-1-yl}pyrrolidin-1-yl]prop-2-en-1-one

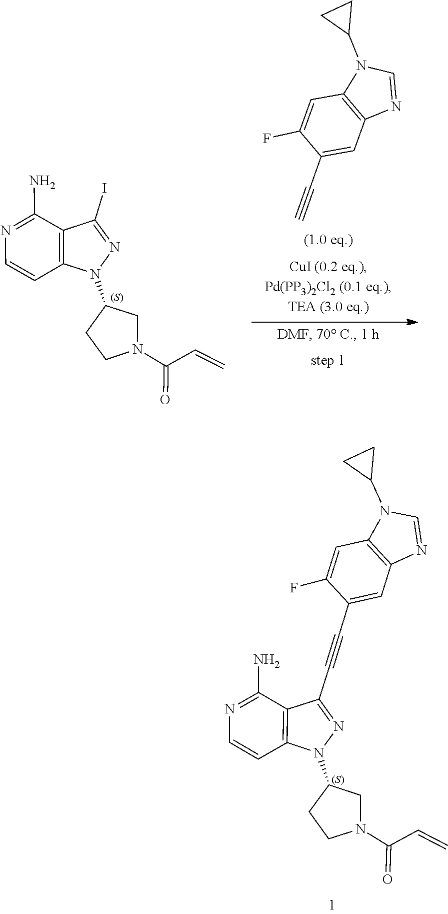

1-[(3S)-3-{4-amino-3-[2-(1-cyclopropyl-6-fluoro-1,3-benzodiazol-5-yl)ethynyl]pyrazolo[4,3-c]pyridin-1-yl}pyrrolidin-1-yl]prop-2-en-1-one. MS ESI calculated for C₂₅H₂₂FN₇O [M+H]⁺ 456.19, found 456.15; ¹H NMR (400 MHz, DMSO-d₆) δ 8.37 (s, 1H), 8.08 (s, 1H), 7.81-7.79 (m, 1H), 7.67-7.62 (m, 1H), 7.02-6.95 (m, 1H), 6.74-6.44 (m, 3H), 6.18 (m, 1H), 5.70 (m, 1H), 5.54-5.33 (m, 1H), 4.14-4.05 (m, 1H), 4.00-3.46 (m, 5H), 2.44-2.27 (m, 1H), 1.18-0.96 (m, 4H).

### Example 150: 1-[(3S)-3-{4-Amino-3-[2-(1-ethyl-6-fluoro-1,3-benzodiazol-5-yl)ethynyl]pyrazolo[4,3-c]pyridin-1-yl}pyrrolidin-1-yl]prop-2-en-1-one

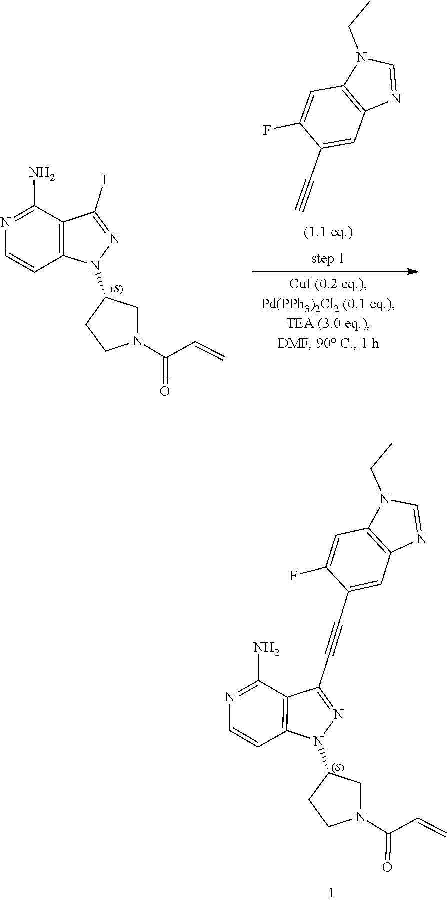

1-[(3S)-3-{4-amino-3-[2-(1-ethyl-6-fluoro-1,3-benzodiazol-5-yl)ethynyl]pyrazolo[4,3-c]pyridin-1-yl}pyrrolidin-1-yl]prop-2-en-1-one. MS ESI calculated for C₂₄H₂₂FN₇O [M+H]⁺, 444.19, found 444.15; ¹H NMR (400 MHz, DMSO-d₆) δ 8.41 (s, 1H), 8.07 (dd, J=6.4, 2.2 Hz, 1H), 7.80 (dd, J=10.7, 8.0 Hz, 2H), 6.97-4.94 (m, 1H), 6.77-6.40 (m, 3H), 6.23-6.16 (m, 1H), 5.76-5.58 (m, 1H), 5.53-5.35 (m, 1H), 4.29 (q, J=7.2 Hz, 2H), 4.17-3.54 (m, 4H), 2.46-2.26 (m, 2H), 1.42 (t, J=7.2 Hz, 3H).

### Example 151: 1-((2S,4S)-4-(4-amino-3-((1-methyl-6-fluoro-1H-benzo[d]imidazol-5-yl)ethynyl)-1H-pyrazolo[3,4-d]pyrimidin-1-yl)-2-methylpyrrolidin-1-yl)prop-2-en-1-one

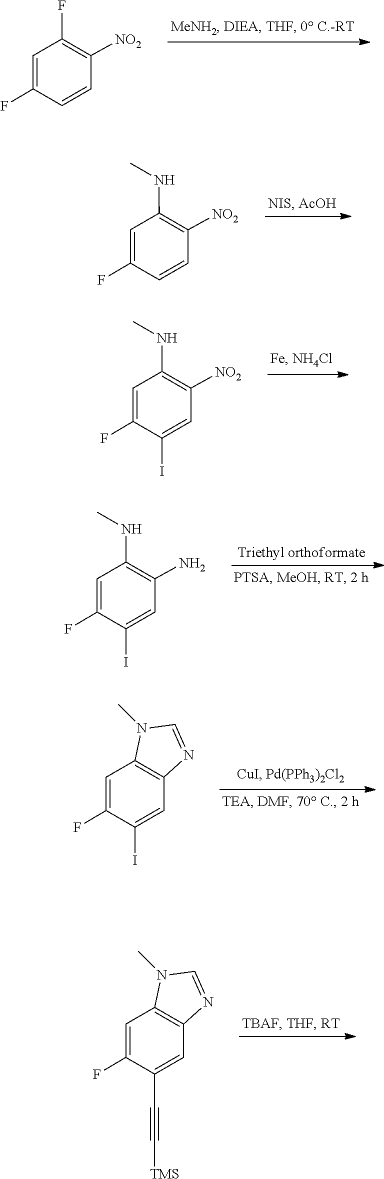

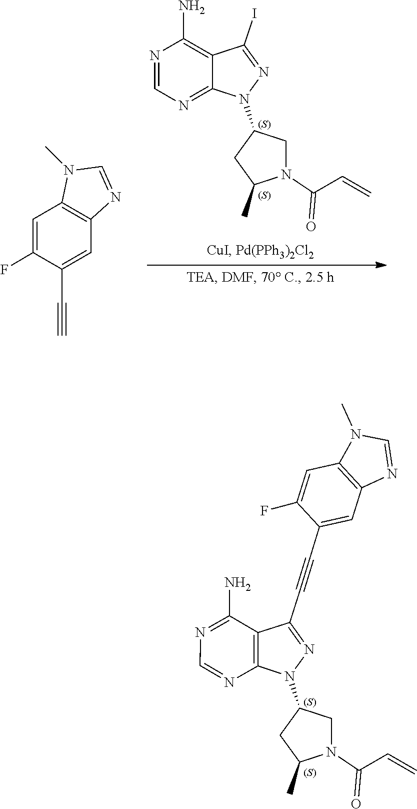

1-((2S,4S)-4-(4-amino-3-((1-methyl-6-fluoro-1H-benzo[d]imidazol-5-yl)ethynyl)-1H-pyrazolo[3,4-d]pyrimidin-1-yl)-2-methylpyrrolidin-1-yl)prop-2-en-1-one. ¹H NMR (400 MHz, DMSO-d₆): 8.3s (s, 1H), 8.29 (s, 1H), 8.14 (d, J=6.4 Hz, 1H), 7.70 (d, J=12.0 Hz, 1H), 6.51-6.73 (m, 1H), 6.11-6.20 (m, 1H), 5.57-5.73 (m, 2H), 4.38-4.54 (m, 1H), 3.96-4.16 (m, 1H), 3.90 (d, J=6.4 Hz, 1H), 3.84 (s, 3H), 2.67-2.75 (m, 1H), 2.10-2.22 (m, 1H). 1.28-1.30 (m, 3H). [M+H] Calcd.: 445.2; Found, 445.2.

### Example 152: 1-[(3S)-3-{4-Amino-3-[2-(1-ethyl-6-fluoro-1,3-benzodiazol-5-yl)ethynyl]pyrazolo[3,4-d]pyrimidin-1-yl}pyrrolidin-1-yl]prop-2-en-1-one

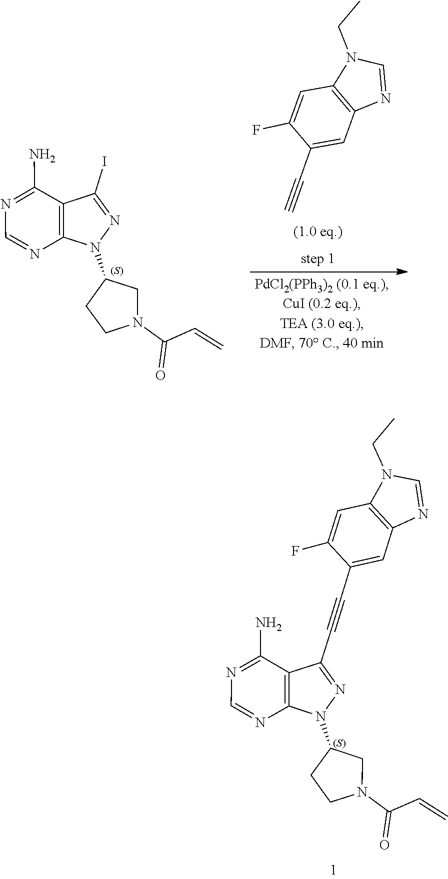

1-[(3S)-3-{4-amino-3-[2-(1-ethyl-6-fluoro-1,3-benzodiazol-5-yl)ethynyl]pyrazolo[3,4-d]pyrimidin-1-yl}pyrrolidin-1-yl]prop-2-en-1-one. MS ESI calculated for C₂₃H₂₁FN₈O [M+H]⁺, 445.18, found 445.10; ¹H NMR (300 MHz, DMSO-d₆) δ 8.42 (s, 1H), 8.31 (s, 1H), 8.16 (dd, J=6.4, 2.2 Hz, 1H), 7.78 (d, J=10.0 Hz, 1H), 6.65-6.59 (m, 1H), 6.19-6.11 (m, 1H), 5.80-5.40 (m, 2H), 4.30-4.26 (m, 2H), 4.18-3.61 (m, 4H), 2.41-2.35 (m, 2H), 1.43 (t, J=7.2 Hz, 3H).

### Example 153: 1-[(3S)-3-{4-Amino-3-[2-(1-ethyl-6,7-difluoro-1,3-benzodiazol-5-yl)ethynyl]pyrazolo[3,4-d]pyrimidin-1-yl}pyrrolidin-1-yl]prop-2-en-1-one

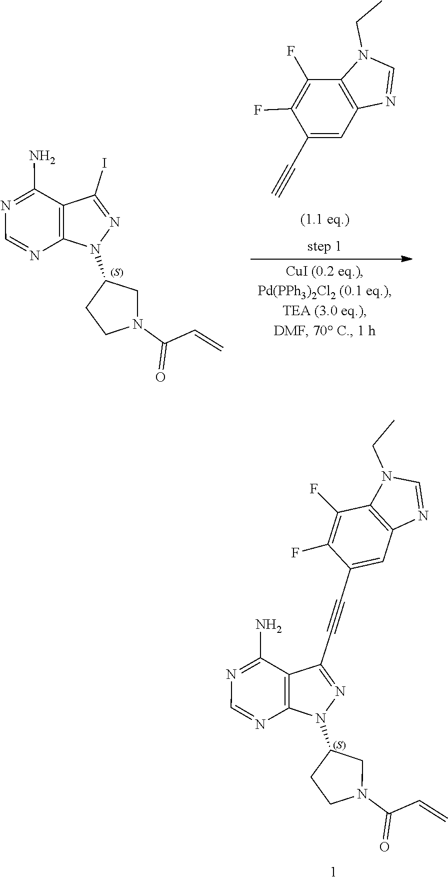

1-[(3S)-3-{4-amino-3-[2-(1-ethyl-6,7-difluoro-1,3-benzodiazol-5-yl)ethynyl]pyrazolo[3,4-d]pyrimidin-1-yl}pyrrolidin-1-yl]prop-2-en-1-one. MS ESI calculated for C₂₃H₂₀F₂N₈O [M+H]⁺, 463.17, found 463.15; ¹H NMR (300 MHz, CD₃OD) δ 8.27-8.22 (m, 2H), 7.76 (s, 1H), 6.66-6.56 (m, 1H), 6.31-6.25 (m, 1H), 5.77 (s, 1H), 5.55 (s, 1H), 4.45 (m, 2H), 4.28-3.47 (m, 4H), 2.55 (m, 2H), 1.55 (m, 3H).

### Example 154: 1-[(3S)-3-{4-Amino-3-[2-(6,7-difluoro-1-methyl-1,3-benzodiazol-5-yl)ethynyl]pyrazolo[3,4-d]pyrimidin-1-yl}pyrrolidin-1-yl]prop-2-en-1-one

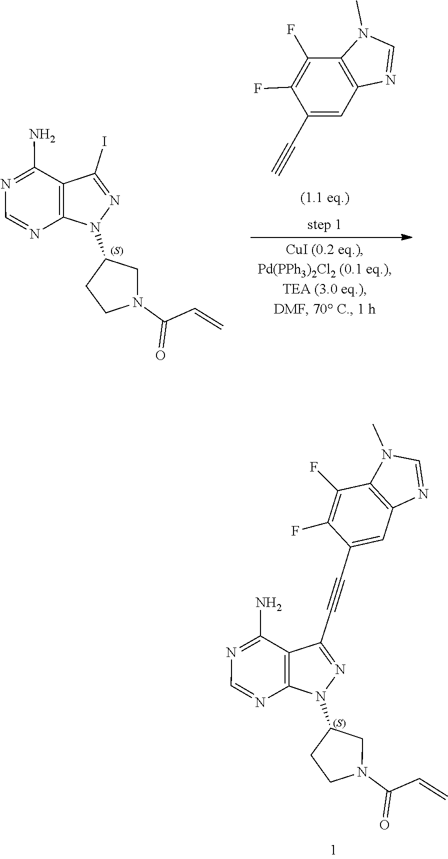

1-[(3S)-3-{4-amino-3-[2-(6,7-difluoro-1-methyl-1,3-benzodiazol-5-yl)ethynyl]pyrazolo[3,4-d]pyrimidin-1-yl}pyrrolidin-1-yl]prop-2-en-1-one. MS ESI calculated for C₂₂H₁₈F₂N₈O [M+H]⁺, 449.16, found 449.10; ¹H NMR (400 MHz, DMSO-d₆) δ 8.32 (d, J=18.6 Hz, 2H), 8.01 (s, 1H), 6.66-6.61 (m, 1H), 6.20-6.16 (m, 1H), 5.78-5.40 (m, 2H), 4.19-3.53 (m, 7H), 2.49-2.28 (m, 2H).

### Example 155: 1-[(3S)-3-{4-Amino-3-[2-(1-ethyl-6,7-difluoro-1,3-benzodiazol-5-yl)ethynyl]pyrazolo[4,3-c]pyridin-1-yl}pyrrolidin-1-yl]prop-2-en-1-one

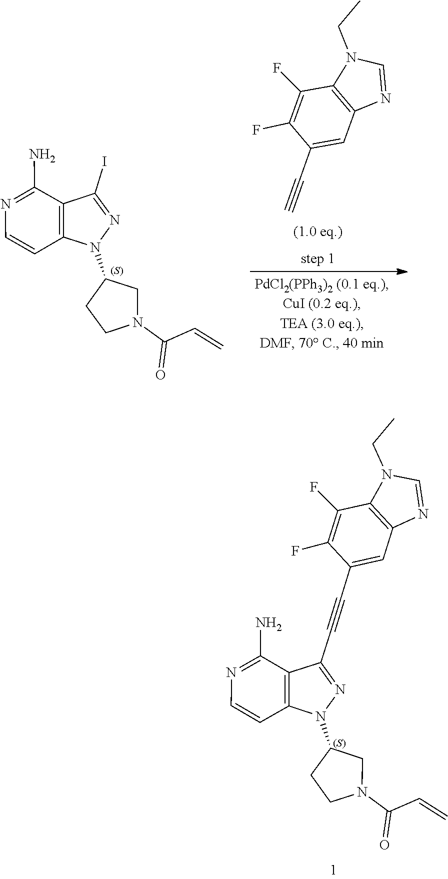

1-[(3S)-3-{4-amino-3-[2-(1-ethyl-6,7-difluoro-1,3-benzodiazol-5-yl)ethynyl]pyrazolo[4,3-c]pyridin-1-yl}pyrrolidin-1-yl]prop-2-en-1-one. MS ESI calculated for C₂₄H₂₁F₂N₇O [M+H]⁺, 462.18, found 462.10; ¹H NMR (300 MHz, DMSO-d₆) δ 8.48 (s, 1H), 7.97 (s, 1H), 7.02 (s, 1H), 6.89-6.26 (m, 3H), 6.19-6.11 (m, 1H), 5.71-5.65 (m, 1H), 5.59-5.31 (m, 1H), 4.40 (q, J=7.2 Hz, 2H), 4.23-3.52 (m, 4H), 2.40-2.35 (m, 2H), 1.47 (t, J=7.1 Hz, 3H).

### Example 156: 1-[(3S)-3-{4-Amino-3-[2-(6,7-difluoro-1-methyl-1,3-benzodiazol-5-yl)ethynyl]pyrazolo[4,3-c]pyridin-1-yl}pyrrolidin-1-yl]prop-2-en-1-one

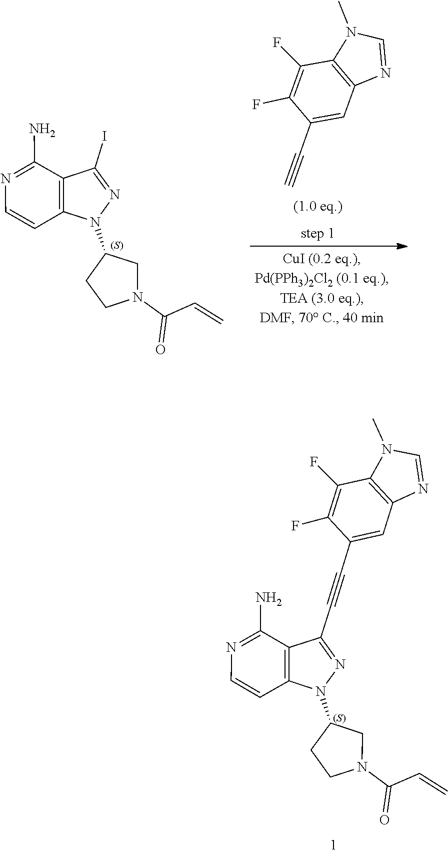

1-[(3S)-3-{4-amino-3-[2-(6,7-difluoro-1-methyl-1,3-benzodiazol-5-yl)ethynyl]pyrazolo[4,3-c]pyridin-1-yl}pyrrolidin-1-yl]prop-2-en-1-one. MS ESI calculated for C₂₃H₁₉F₂N₇O [M+H]⁺, 448.16, found 448.10; ¹H NMR (300 MHz, DMSO-d₆) δ 8.33 (s, 1H), 7.93-7.79 (m, 2H), 6.98-6.95 (m, 1H), 6.68-6.58 (m, 1H), 6.47 (s, 2H), 6.21-6.13 (m, 1H), 5.73-5.64 (m, 1H), 5.56-5.37 (m, 1H), 4.19-3.55 (m, 7H), 2.50-2.33 (m, 2H).

### Example 157: 2-[(2R,4S)-4-{4-Amino-3-[2-(1-ethyl-6,7-difluoro-1,3-benzodiazol-5-yl)ethynyl]pyrazolo[3,4-d]pyrimidin-1-yl}-1-(prop-2-enoyl)pyrrolidin-2-yl]acetonitrile

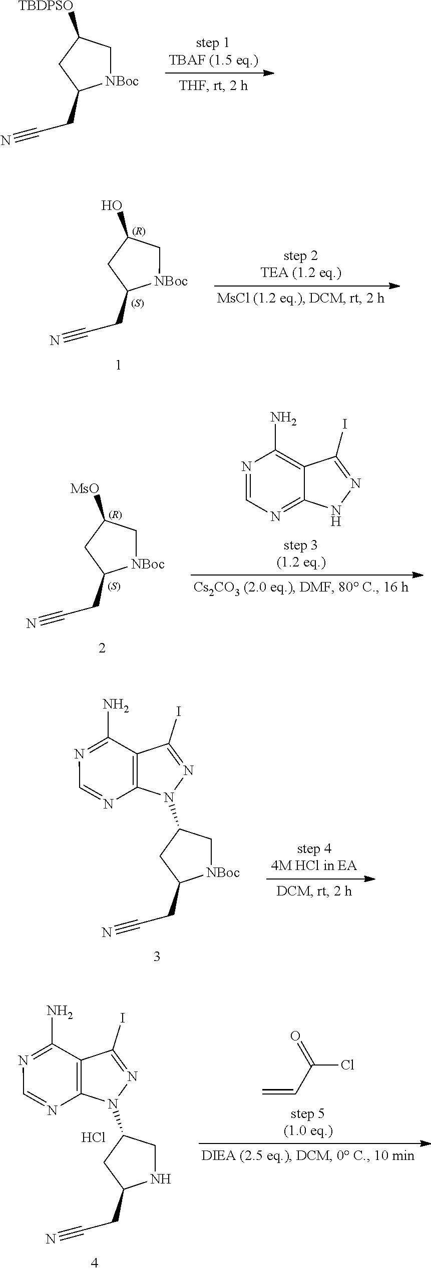

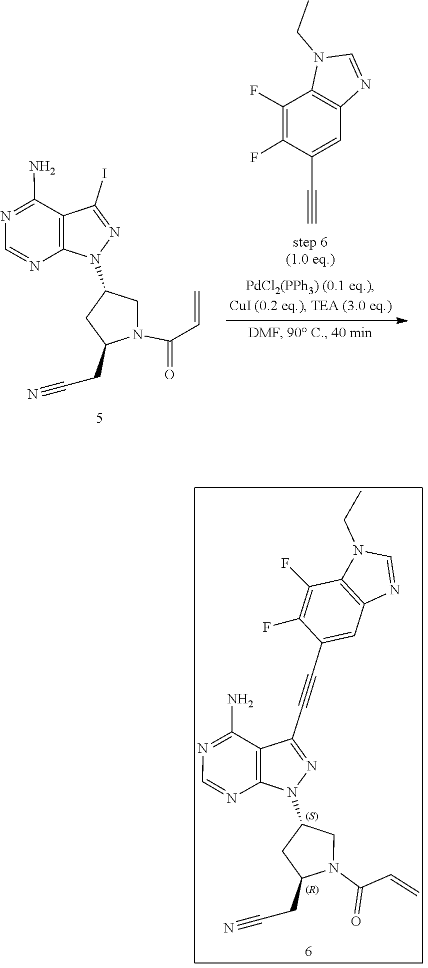

2-[(2R,4S)-4-{4-amino-3-[2-(1-ethyl-6,7-difluoro-1,3-benzodiazol-5-yl)ethynyl]pyrazolo[3,4-d]pyrimidin-1-yl}-1-(prop-2-enoyl)pyrrolidin-2-yl]acetonitrile. MS ESI calculated for C₂₅H₂₁F₂N₉O [M+H]⁺, 502.18, found 502.15; ¹H NMR (400 MHz, CDCl₃) δ 8.42 (s, 1H), 7.92 (s, 1H), 6.45-6.39 (m, 2H), 6.19 (s, 2H), 5.80-5.70 (m, 2H), 4.80-4.75 (m, 1H), 4.46-4.38 (m, 2H), 4.28-4.16 (m, 2H), 3.32-3.28 (m, 1H), 3.03-2.96 (m, 1H), 2.85-2.80 (m, 1H), 2.56-2.51 (m, 1H), 1.61-1.59 (m, 3H).

### Example 158: 2-[(2R,4S)-4-{4-Amino-3-[2-(6,7-difluoro-1-methyl-1,3-benzodiazol-5-yl)ethynyl]pyrazolo[3,4-d]pyrimidin-1-yl}-1-(prop-2-enoyl)pyrrolidin-2-yl]acetonitrile

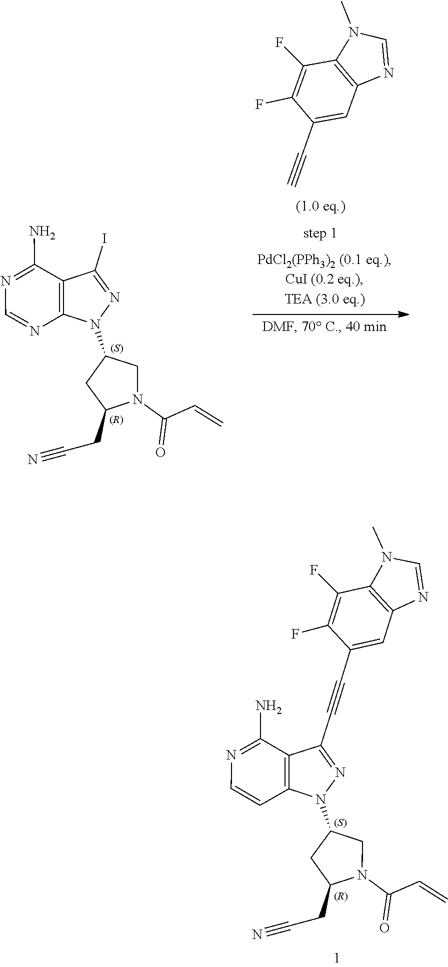

2-[(2R,4S)-4-{4-amino-3-[2-(6,7-difluoro-1-methyl-1,3-benzodiazol-5-yl)ethynyl]pyrazolo[3,4-d]pyrimidin-1-yl}-1-(prop-2-enoyl)pyrrolidin-2-yl]acetonitrile. MS ESI calculated for C₂₄H₁₉F₂N₉O [M+H]⁺, 488.17, found 488.10; ¹H NMR (300 MHz, DMSO-d₆) δ 8.33 (d, J=11.2 Hz, 2H), 8.02 (dd, J=5.3, 1.4 Hz, 1H), 6.90-6.54 (m, 1H), 6.20 (dd, J=16.7, 2.3 Hz, 1H), 5.84-5.60 (m, 2H), 4.69-4.62 (m, 1H), 4.25-4.08 (m, 1H), 4.02-3.96 (m, 3H), 3.21-3.28 (m, 1H), 3.03-2.95 (m, 1H), 2.79-2.71 (m, 1H), 2.42-2.37 (m, 2H).

### Example 159: 2-((2R,4S)-1-Acryloyl-4-(4-amino-3-((6,7-difluoro-1-methyl-1H-benzo[d]imidazol-5-yl)ethynyl)-1H-pyrazolo[4,3-c]pyridin-1-yl)pyrrolidin-2-yl)acetonitrile

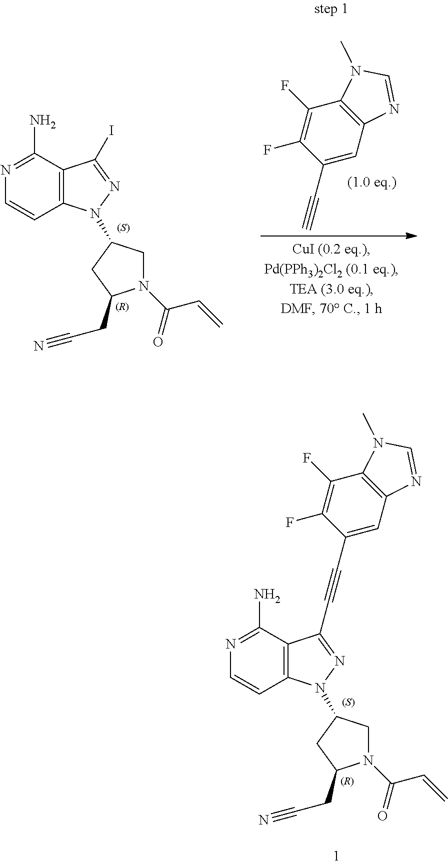

2-((2R,4S)-1-acryloyl-4-(4-amino-3-((6,7-difluoro-1-methyl-1H-benzo[d]imidazol-5-yl)ethynyl)-1H-pyrazolo[4,3-c]pyridin-1-yl)pyrrolidin-2-yl)acetonitrile. MS ESI calculated for C₂₅H₂₀F₂N₈O [M+H]⁺, 487.17, found 487.15; ¹H NMR (300 MHz, DMSO-d₆) δ 8.35 (s, 1H), 7.99-7.90 (m, 1H), 7.84 (d, J=6.1 Hz, 1H), 6.99 (d, J=6.1 Hz, 1H), 6.69-6.41 (m, 3H), 6.20 (dd, J=16.6, 2.4 Hz, 1H), 5.82-5.53 (m, 2H), 4.55 (s, 1H), 4.14-4.01 (m, 1H), 4.02-3.95 (m, 4H), 3.22-2.97 (m, 2H), 2.81-2.66 (m, 1H), 2.44-2.25 (m, 1H).

### Example 160: 2-[(2R,4S)-4-{4-Amino-3-[2-(1-ethyl-6,7-difluoro-1,3-benzodiazol-5-yl)ethynyl]pyrazolo[4,3-c]pyridin-1-yl}-1-(prop-2-enoyl)pyrrolidin-2-yl]acetonitrile

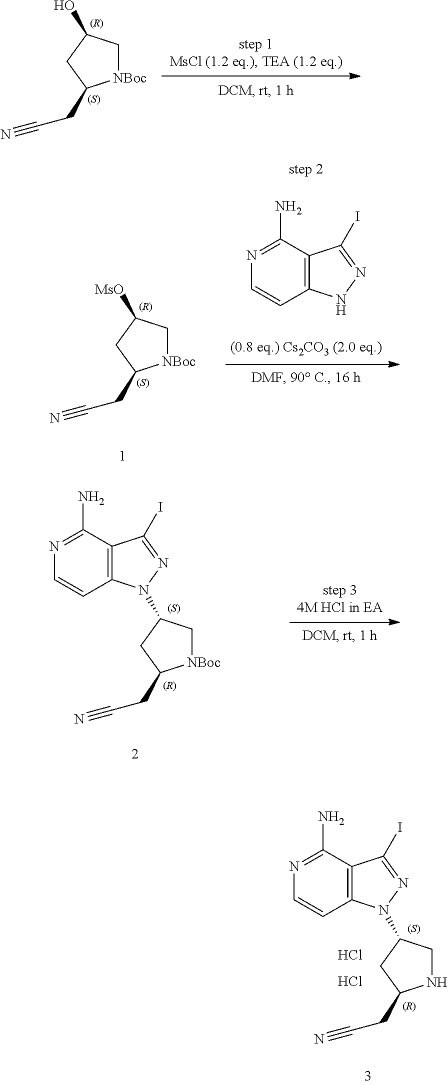

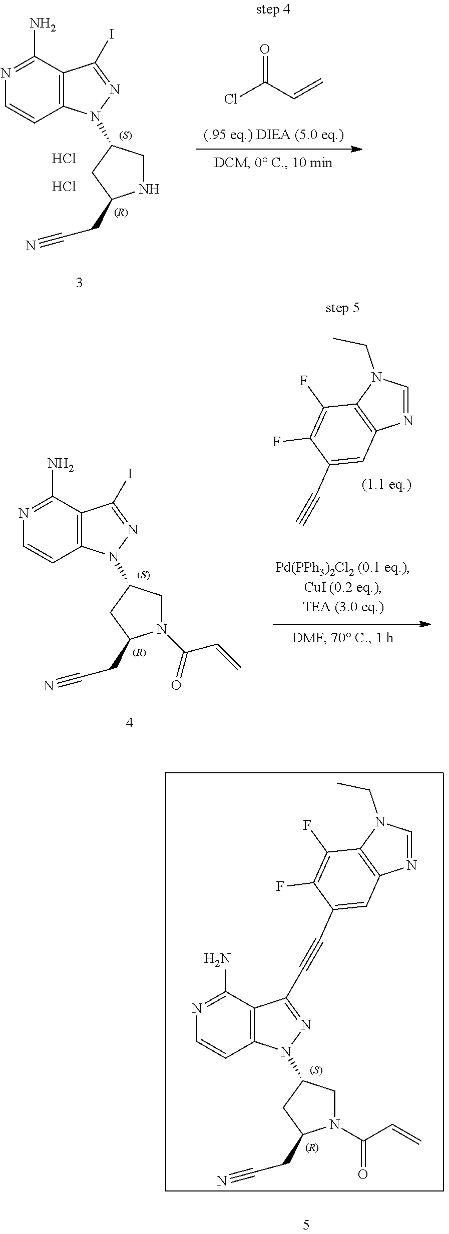

2-[(2R,4S)-4-{4-amino-3-[2-(1-ethyl-6,7-difluoro-1,3-benzodiazol-5-yl)ethynyl]pyrazolo[4,3-c]pyridin-1-yl}-1-(prop-2-enoyl)pyrrolidin-2-yl]acetonitrile. MS ESI calculated for C₂₆H₂₂F₂N₈O [M+H]⁺, 501.19, found 501.15; ¹H NMR (400 MHz, CDCl₃) δ 7.92 (s, 1H), 7.87 (d, J=8 Hz, 1H), 7.80 (d, J=8 Hz, 1H), 6.75 (d, J=8 Hz, 1H), 6.43-6.40 (m, 2H), 6.09 (s, 2H), 5.77 (dd, J=4, 4 Hz, 1H), 5.54-5.51 (m, 1H), 4.76 (s, 1H), 4.42 (q, J=60 Hz, 2H), 4.28-4.23 (m, 1H), 4.14-4.09 (m, 1H), 3.47-3.41 (m, 1H), 3.00-2.97 (m, 1H), 2.73-2.68 (m, 1H), 2.53-2.50 (m, 1H), 1.59 (t, J=32 Hz, 3H).

### Example 161: 2-[(2R,4S)-4-{4-Amino-3-[2-(6-chloro-7-fluoro-1-methyl-1,3-benzodiazol-5-yl)ethynyl]pyrazolo[3,4-d]pyrimidin-1-yl}-1-(prop-2-enoyl)pyrrolidin-2-yl]acetonitrile

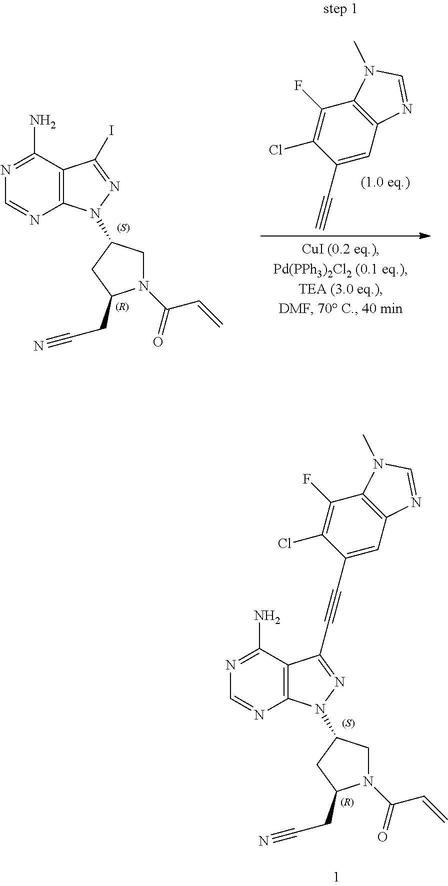

2-[(2R,4S)-4-{4-amino-3-[2-(6-chloro-7-fluoro-1-methyl-1,3-benzodiazol-5-yl)ethynyl]pyrazolo[3,4-d]pyrimidin-1-yl}-1-(prop-2-enoyl)pyrrolidin-2-yl]acetonitrile. MS ESI calculated for C₂₄H₁₉ClFN₉O [M+H]⁺, 504.14, 506.14, found 504.10, 506.10; ¹H NMR (300 MHz, CDCl₃) δ 8.40 (s, 1H), 7.94 (d, J=12.7 Hz, 2H), 6.42 (d, J=6.1 Hz, 2H), 6.25 (s, 2H), 5.76-5.68 (m, 2H), 4.80 (d, J=7.0 Hz, 1H), 4.31-4.19 (m, 2H), 4.08-4.01 (m, 3H), 3.34-3.26 (m, 1H), 3.06-3.00 (m, 1H), 2.86-2.80 (m, 1H), 2.58-2.50 (m, 1H).

### Example 162: 2-[(2R,4S)-4-{4-Amino-3-[2-(6-chloro-1-ethyl-7-fluoro-1,3-benzodiazol-5-yl)ethynyl]pyrazolo[4,3-c]pyridin-1-yl}-1-(prop-2-enoyl)pyrrolidin-2-yl]acetonitrile

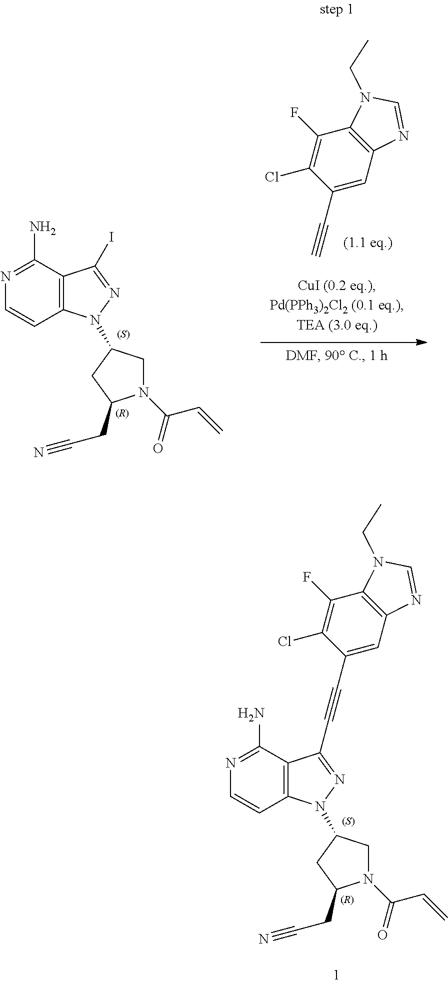

2-[(2R,4S)-4-{4-amino-3-[2-(6-chloro-1-ethyl-7-fluoro-1,3-benzodiazol-5-yl)ethynyl]pyrazolo[4,3-c]pyridin-1-yl}-1-(prop-2-enoyl)pyrrolidin-2-yl]acetonitrile. MS ESI calculated for C₂₆H₂₂ClFN₈O [M+H]⁺, 517.16, found 517.10; ¹H NMR (400 MHz, CDCl₃) δ 7.94-7.89 (m, 3H), 6.76 (d, J=8 Hz, 1H), 6.48-6.42 (m, 2H), 5.97 (s, 2H), 5.79-5.71 (m, 1H), 5.55-5.45 (m, 1H), 4.78-7.72 (m, 2H), 4.29-4.15 (m, 2H), 3.45-3.38 (m, 1H), 3.05-2.98 (m, 1H), 2.83-2.71 (m, 1H), 2.56-2.50 (m, 1H), 1.62-1.59 (m, 3H), 1.28-1.22 (m, 1H).

### Example 163: 1-((2S,4S)-4-(4-amino-3-((1-ethyl-6-fluoro-1H-benzo[d]imidazol-5-yl)ethynyl)-1H-pyrazolo[3,4-d]pyrimidin-1-yl)-2-methylpyrrolidin-1-yl)prop-2-en-1-one

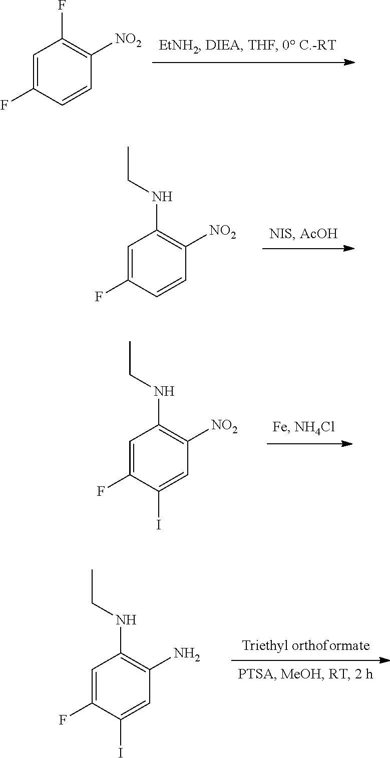

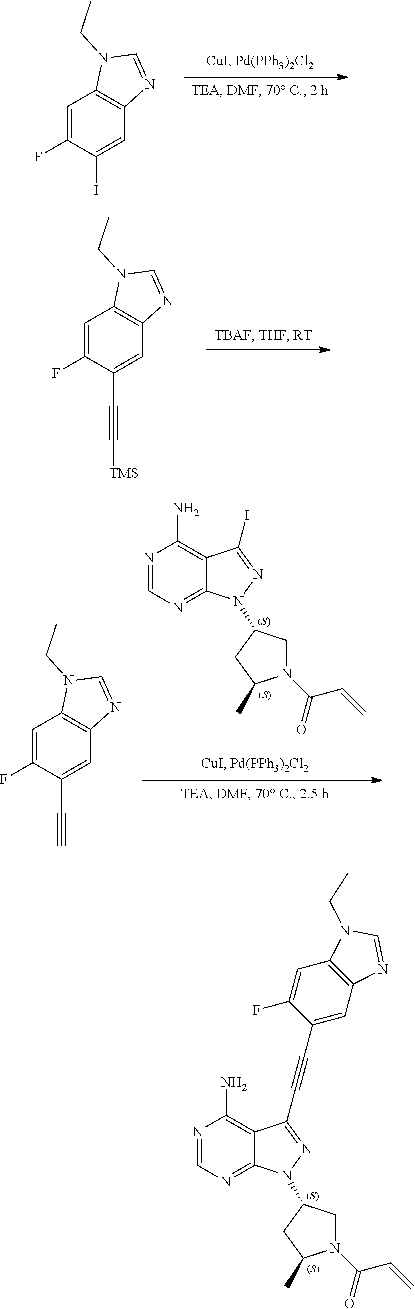

1-((2S,4S)-4-(4-amino-3-((1-ethyl-6-fluoro-1H-benzo[d]imidazol-5-yl)ethynyl)-1H-pyrazolo[3,4-d]pyrimidin-1-yl)-2-methylpyrrolidin-1-yl)prop-2-en-1-one. ¹H NMR (400 MHz, DMSO-d₆): 8.40 (s, 1H), 8.29 (s, 1H), 8.14 (d, J=12.0 Hz, 1H), 7.78 (d, J=8.0 Hz, 1H), 6.78-6.51 (m, 1H), 6.16 (t, J=13.2 Hz, 1H), 5.76-5.52 (m, 2H), 4.57-4.37 (m, 3H), 4.19-3.87 (m, 2H), 2.79-2.68 (m, 1H), 2.21-2.09 (m, 1H), 1.42 (t, J=7.2 Hz, 3H), 1.28-1.30 (m, 3H). [M+H] Calcd.: 455.2; Found, 459.2.

### Example 164: 1-((2S,4S)-4-(4-amino-3-((6-chloro-1-methyl-1H-benzo[d]imidazol-5-yl)ethynyl)-1H-pyrazolo[3,4-d]pyrimidin-1-yl)-2-methylpyrrolidin-1-yl)prop-2-en-1-one

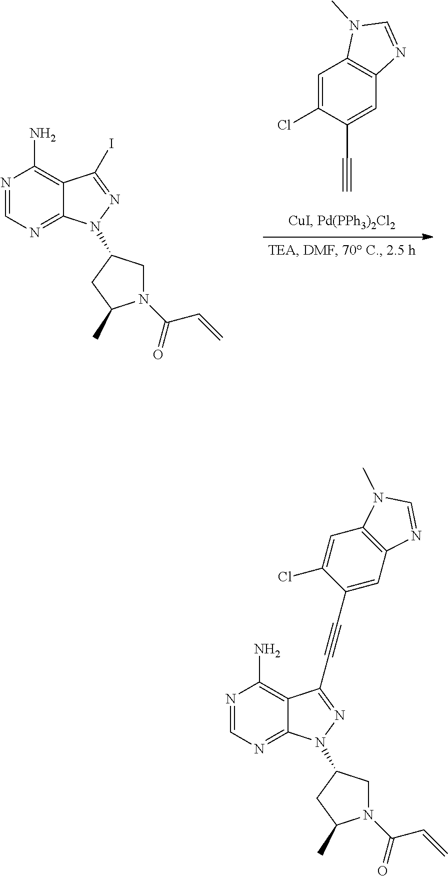 [SMILES: C=CC(=O)N1C[C@@H](n2nc(I)c3c(N)ncnc32)C[C@@H]1C ; C#Cc1cc2ncn(C)c2cc1Cl ; C=CC(=O)N1C[C@@H](n2nc(C#Cc3cc4ncn(C)c4cc3Cl)c3c(N)ncnc32)C[C@@H]1C]

1-((2S,4S)-4-(4-amino-3-((6-chloro-1-methyl-1H-benzo[d]imidazol-5-yl)ethynyl)-1H-pyrazolo[3,4-d]pyrimidin-1-yl)-2-methylpyrrolidin-1-yl)prop-2-en-1-one. ¹H NMR (400 MHz, DMSO-d₆): 8.39 (s, 1H), 8.34 (s, 1H), 8.19 (s, 1H), 7.98 (s, 1H), 7.93-7.29 (m, 2H), 6.75-6.50 (m, 1H), 6.10 (t, J=13.2 Hz, 1H), 5.74-5.57 (m, 2H), 4.58-4.35 (m, 1H), 4.10-3.90 (m, 2H), 3.86 (s, 3H), 2.81-2.64 (m, 1H), 2.30-2.09 (m, 1H), 1.28-1.30 (m, 3H). [M+H] Calcd.: 461.2; Found, 461.2.

### Example 165: 1-((2S,4S)-4-(4-amino-3-((6-fluoro-1-methyl-1H-benzo[d]imidazol-5-yl)ethynyl)-1H-pyrazolo[4,3-c]pyridin-1-yl)-2-methylpyrrolidin-1-yl)prop-2-en-1-one

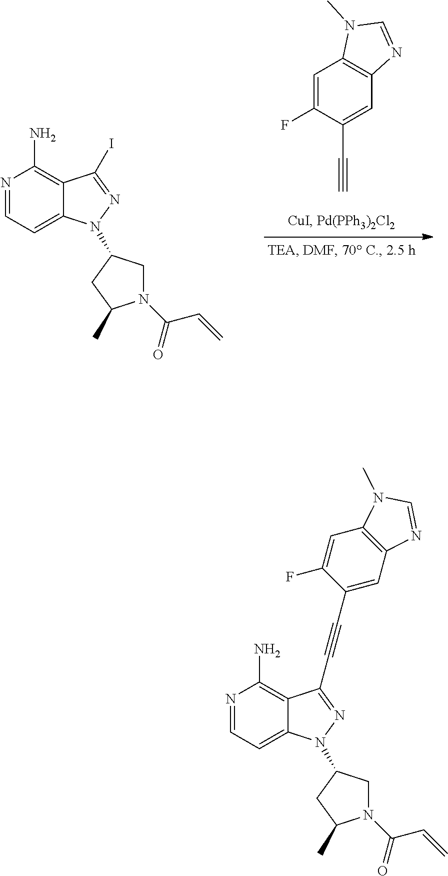 [SMILES: C=CC(=O)N1C[C@@H](n2nc(I)c3c(N)nccc32)C[C@@H]1C ; C#Cc1cc2ncn(C)c2cc1F ; C=CC(=O)N1C[C@@H](n2nc(C#Cc3cc4ncn(C)c4cc3F)c3c(N)nccc32)C[C@@H]1C]

1-((2S,4S)-4-(4-amino-3-((6-fluoro-1-methyl-1H-benzo[d]imidazol-5-yl)ethynyl)-1H-pyrazolo[4,3-c]pyridin-1-yl)-2-methylpyrrolidin-1-yl)prop-2-en-1-one. ¹H NMR (400 MHz, DMSO-d₆): 8.33 (s, 1H), 8.05 (d, J=6.0 Hz, 1H), 7.80 (s, 1H), 7.71 (d, J=12.0 Hz, 1H), 7.04-7.02 (m, 1H), 6.72-6.47 (m, 3H), 6.11-6.20 (m, 1H), 5.73-5.62 (m, 1H), 5.52 (t, J=6.8 Hz, 1H), 4.53-4.40 (m, 1H), 4.15-3.81 (m, 5H), 2.75-2.60 (m, 1H), 2.24-2.08 (m, 1H), 1.31-1.29 (m, 3H). [M+H] Calcd.: 444.1; Found, 444.1.

### Example 166: 1-((2S,4S)-4-(4-amino-3-((1-ethyl-6-fluoro-1H-benzo[d]imidazol-5-yl)ethynyl)-1H-pyrazolo[4,3-c]pyridin-1-yl)-2-methylpyrrolidin-1-yl)prop-2-en-1-one

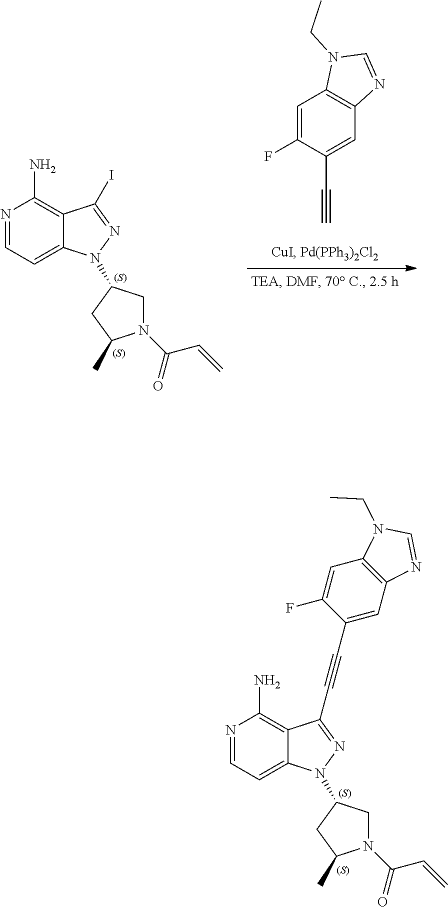

1-((2S,4S)-4-(4-amino-3-((1-ethyl-6-fluoro-1H-benzo[d]imidazol-5-yl)ethynyl)-1H-pyrazolo[4,3-c]pyridin-1-yl)-2-methylpyrrolidin-1-yl)prop-2-en-1-one. ¹H NMR (400 MHz, DMSO-d₆): 8.48 (s, 1H), 8.04-8.08 (m, 1H), 7.81-7.69 (m, 2H), 7.07-7.02 (m, 1H), 6.73-6.42 (m, 3H), 6.16 (t, J=13.2 Hz, 1H), 5.78-5.71 (m, 1H), 5.60-5.49 (m, 1H), 4.58-4.49 (m, 1H), 4.35-4.32 (m, 2H), 4.22-3.78 (m, 2H), 2.80-2.70 (m, 1H), 2.20-2.06 (m, 1H), 1.46 (t, J=7.2 Hz, 3H), 1.31-1.30 (m, 3H). [M+H] Calcd.: 458.2; Found, 458.2.

### Example 167: 1-((2S,4S)-4-(4-amino-3-((6-chloro-1-methyl-1H-benzo[d]imidazol-5-yl)ethynyl)-1H-pyrazolo[4,3-c]pyridin-1-yl)-2-methylpyrrolidin-1-yl)prop-2-en-1-one

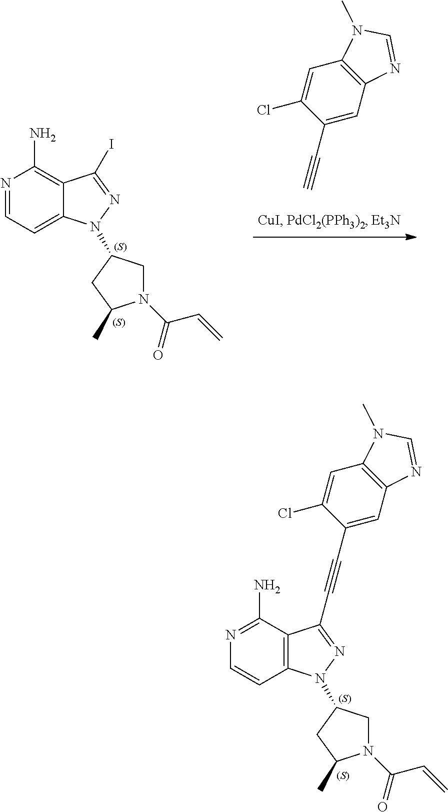

1-((2S,4S)-4-(4-amino-3-((6-chloro-1-methyl-1H-benzo[d]imidazol-5-yl)ethynyl)-1H-pyrazolo[4,3-c]pyridin-1-yl)-2-methylpyrrolidin-1-yl)prop-2-en-1-one. ¹H NMR (400 MHz, DMSO-d₆): 8.36 (s, 1H), 8.11 (s, 1H), 7.98 (s, 1H), 7.81 (d, J=6.4 Hz, 1H), 7.05-7.03 (m, 1H), 6.73-6.53 (m, 3H), 6.17 (t, J=16.4 Hz, 1H), 5.68 (dd, J=10.4, 29.6 Hz, 1H), 5.57-5.51 (m, 1H), 4.55-4.40 (m, 1H), 4.18-3.78 (m, 5H), 2.76-2.59 (m, 1H), 2.33-2.09 (m, 1H), 1.32-1.27 (m, 3H). [M+H] Calcd.: 460.2; Found, 460.2.

### Example 168: 1-((2S,4S)-4-(4-amino-3-((1-ethyl-6-chloro-1H-benzo[d]imidazol-5-yl)ethynyl)-1H-pyrazolo[3,4-d]pyrimidin-1-yl)-2-methylpyrrolidin-1-yl)prop-2-en-1-one

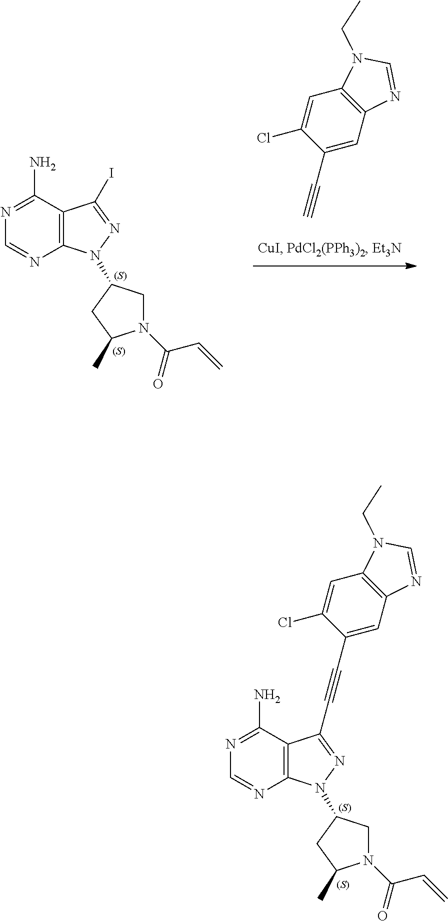

1-((2S,4S)-4-(4-amino-3-((1-ethyl-6-chloro-1H-benzo[d]imidazol-5-yl)ethynyl)-1H-pyrazolo[3,4-d]pyrimidin-1-yl)-2-methylpyrrolidin-1-yl)prop-2-en-1-one. ¹H NMR (400 MHz, DMSO-d₆): 8.50 (br s, 1H), 8.30-8.22 (m, 3H), 8.05 (s, 1H), 6.73-6.51 (m, 2H), 6.21-6.11 (m, 1H), 5.73-5.57 (m, 2H), 4.54-4.37 (m, 1H), 4.34-4.29 (m, 2H), 4.17-3.97 (m, 1H), 3.91 (d, J=7.2 Hz, 1H), 2.72-2.62 (m, 1H), 2.26-2.10 (m, 1H), 1.41 (t, J=7.6 Hz, 3H). 1.31-1.28 (m, 3H). [M+H] Calcd.: 475.2; Found, 475.2.

### Example 169: 1-((2S,4S)-4-(4-amino-3-((6-chloro-1-ethyl-1H-benzo[d]imidazol-5-yl)ethynyl)-1H-pyrazolo[4,3-c]pyridin-1-yl)-2-methylpyrrolidin-1-yl)prop-2-en-1-one

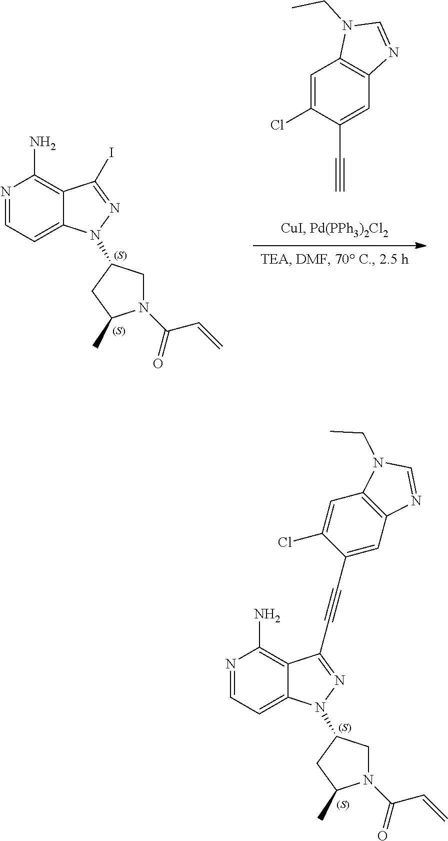

1-((2S,4S)-4-(4-amino-3-((6-chloro-1-ethyl-1H-benzo[d]imidazol-5-yl)ethynyl)-1H-pyrazolo[4,3-c]pyridin-1-yl)-2-methylpyrrolidin-1-yl)prop-2-en-1-one. ¹H NMR (400 MHz, DMSO-d₆): 8.46 (br s, 1H), 8.12 (s, 1H), 8.04 (s, 1H), 7.82 (brs, 1H), 7.04 (s, 1H), 6.73-6.52 (m, 3H), 6.21-6.12 (m, 1H), 5.73-5.62 (m, 1H), 5.56-5.52 (m, 1H), 4.53-4.40 (m, 1H), 4.34-4.29 (m, 2H), 4.18-3.80 (m, 2H), 2.76-2.61 (m, 1H), 2.25-2.08 (m, 1H), 1.41 (t, J=7.2 Hz, 3H), 1.32-1.30 (m, 3H). [M+H] Calcd.: 474.2; Found, 474.2.

### Example 170: 1-((2R,4S)-4-(4-amino-3-((1-ethyl-6-fluoro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one

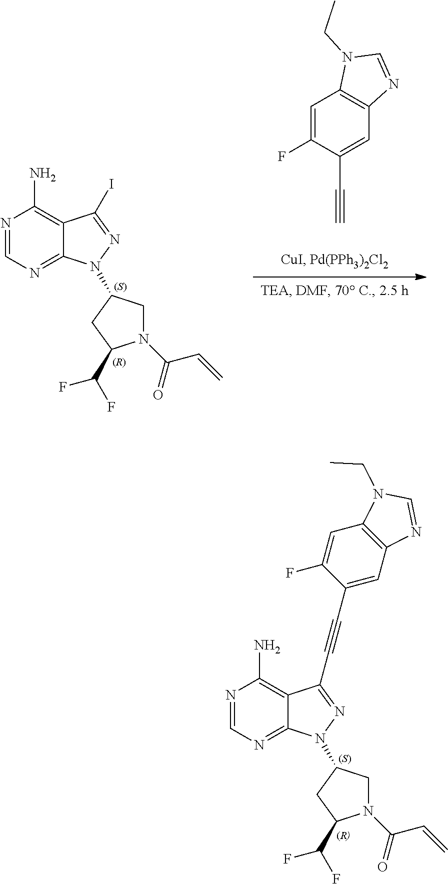

1-((2R,4S)-4-(4-amino-3-((1-ethyl-6-fluoro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one. ¹H NMR (400 MHz, DMSO-d₆): 8.40 (s, 1H), 8.30 (s, 1H), 8.15 (d, J=6.4 Hz, 1H), 7.78 (d, J=10.0 Hz, 1H), 6.85-6.19 (m, 4H), 5.76-5.73 (m, 1H), 5.63-5.58 (m, 1H), 4.98-4.72 (m, 1H), 4.31-4.26 (m, 2H). 4.13-3.80 (m, 2H), 2.80-2.54 (m, 2H), 1.41 (t, J=7.2 Hz, 3H). [M+H] Calcd.: 495.2; Found, 495.2.

### Example 171: 1-((2R,4S)-4-(4-amino-3-((1-ethyl-6-fluoro-1H-benzo[d]imidazol-5-yl)ethynyl)-1H-pyrazolo[4,3-c]pyridin-1-yl)-2-(difluoromethyl)pyrrolidin-1-yl)prop-2-en-1-one

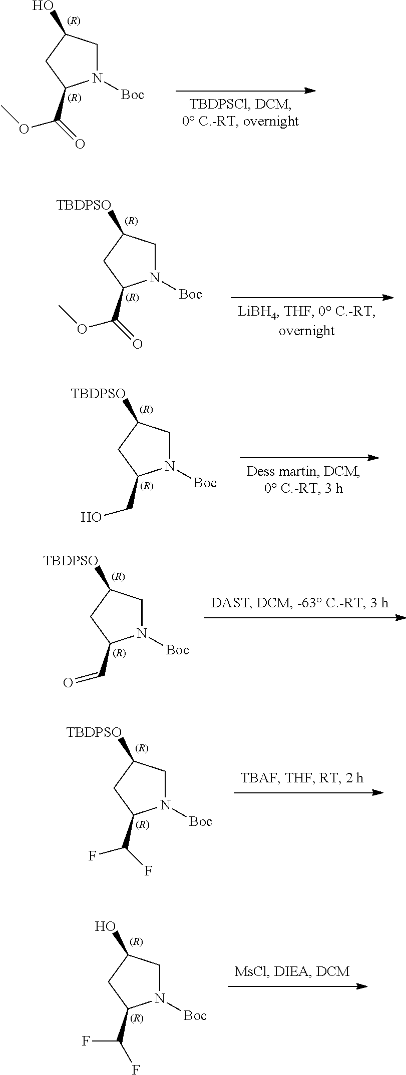

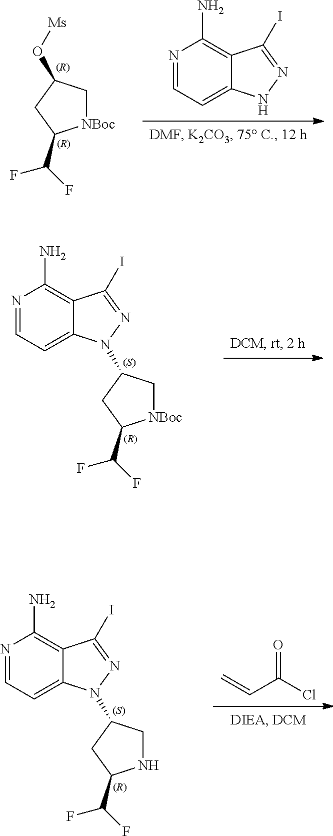

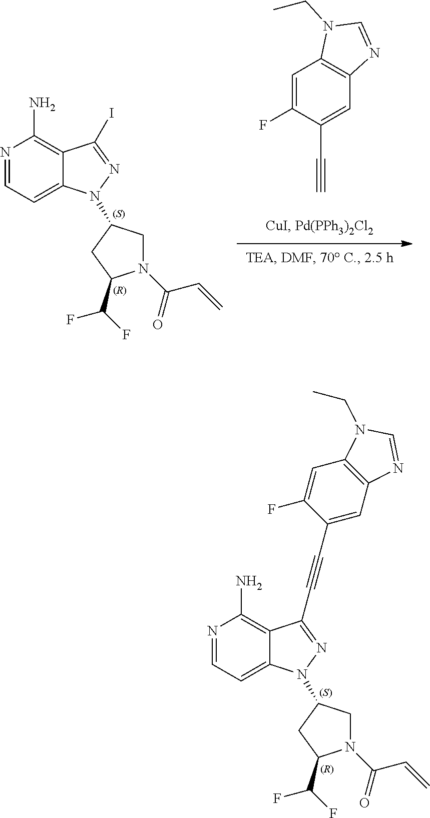

1-((2R,4S)-4-(4-amino-3-((1-ethyl-6-fluoro-1H-benzo[d]imidazol-5-yl)ethynyl)-1H-pyrazolo[4,3-c]pyridin-1-yl)-2-(difluoromethyl)pyrrolidin-1-yl)prop-2-en-1-one. ¹H NMR (400 MHz, DMSO-d₆): 8.51 (s, 1H), 8.07-8.05 (m, 1H), 7.82-7.77 (m, 2H), 6.98 (d, J=6.0 Hz, 1H), 6.81-6.50 (m, 3H), 6.41-6.13 (m, 2H), 5.77-5.72 (m, 1H), 5.50-5.47 (m, 1H), 4.97-4.69 (m, 1H), 4.32-4.26 (m, 2H), 4.14-3.77 (m, 2H), 2.80-2.54 (m, 2H), 1.41 (t, J=7.2 Hz, 3H). [M+H] Calcd.: 494.2; Found, 494.2.

### Example 172: 1-((2R,4S)-4-(4-amino-3-((6-chloro-1-methyl-1H-benzo[d]imidazol-5-yl)ethynyl)-1H-pyrazolo[4,3-c]pyridin-1-yl)-2-(difluoromethyl)pyrrolidin-1-yl)prop-2-en-1-one

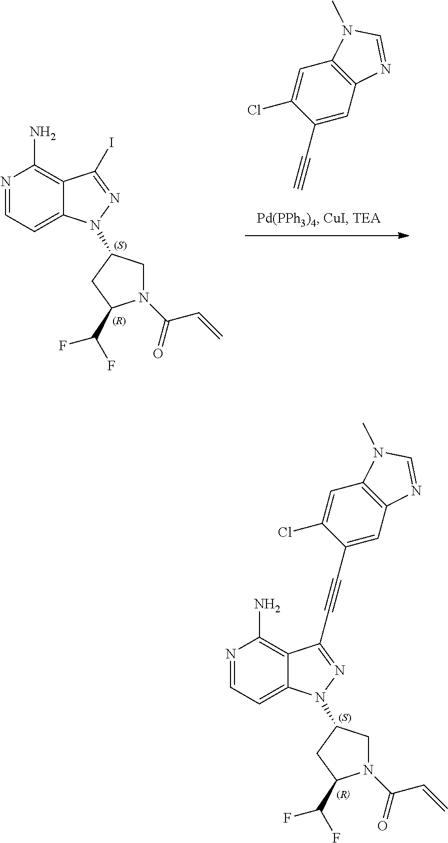

1-((2R,4S)-4-(4-amino-3-((6-chloro-1-methyl-1H-benzo[d]imidazol-5-yl)ethynyl)-1H-pyrazolo[4,3-c]pyridin-1-yl)-2-(difluoromethyl)pyrrolidin-1-yl)prop-2-en-1-one. ¹H NMR (400 MHz, DMSO-d₆): 8.38 (s, 1H), 8.13 (s, 1H), 7.98 (s, 1H), 7.83 (s, 1H), 7.08 (d, J=5.6 Hz, 1H), 6.88-6.74 (m, 2H), 6.65-6.55 (m, 1H), 6.41-6.13 (m, 2H), 5.77-5.73 (m, 1H), 5.53-5.50 (m, 1H), 4.96-4.71 (m, 1H), 4.15-3.78 (m, 5H), 2.81-2.57 (m, 2H). [M+H] Calcd.: 496.1; Found, 496.1.

### Example 173: 2-[(2R,4S)-4-{4-Amino-3-[2-(6-chloro-1-ethyl-7-fluoro-1,3-benzodiazol-5-yl)ethynyl]pyrazolo[3,4-d]pyrimidin-1-yl}-1-(prop-2-enoyl)pyrrolidin-2-yl]acetonitrile

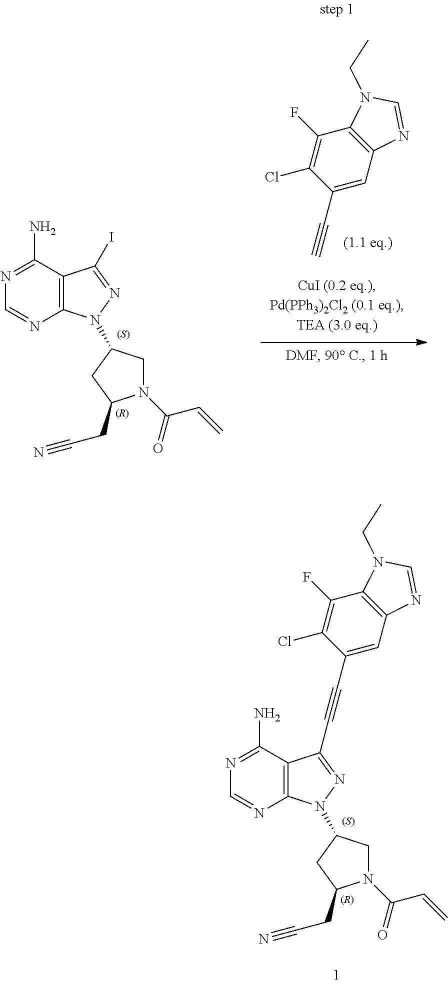

2-[(2R,4S)-4-{4-amino-3-[2-(6-chloro-1-ethyl-7-fluoro-1,3-benzodiazol-5-yl)ethynyl]pyrazolo[3,4-d]pyrimidin-1-yl}-1-(prop-2-enoyl)pyrrolidin-2-yl]acetonitrile. MS ESI calculated for C₂₅H₂₁ClFN₉O [M+H]⁺, 518.15, found 518.25; ¹H NMR (400 MHz, CDCl₃) δ 7.94 (s, 1H), 7.26 (d, J=4 Hz, 2H), 6.41-6.39 (m, 2H), 6.15 (s, 2H), 5.76-5.70 (m, 2H), 4.79-4.77 (m, 1H), 4.43-4.37 (m, 2H), 4.25-4.19 (m, 2H), 3.30-3.25 (m, 1H), 3.06-2.99 (m, 1H), 2.83-2.78 (m, 1H), 2.62-2.51 (m, 1H), 1.61-1.57 (m, 3H).

### Example 174: 2-[(2R,4S)-4-{4-Amino-3-[2-(6-chloro-7-fluoro-1-methyl-1,3-benzodiazol-5-yl)ethynyl]pyrazolo[4,3-c]pyridin-1-yl}-1-(prop-2-enoyl)pyrrolidin-2-yl]acetonitrile

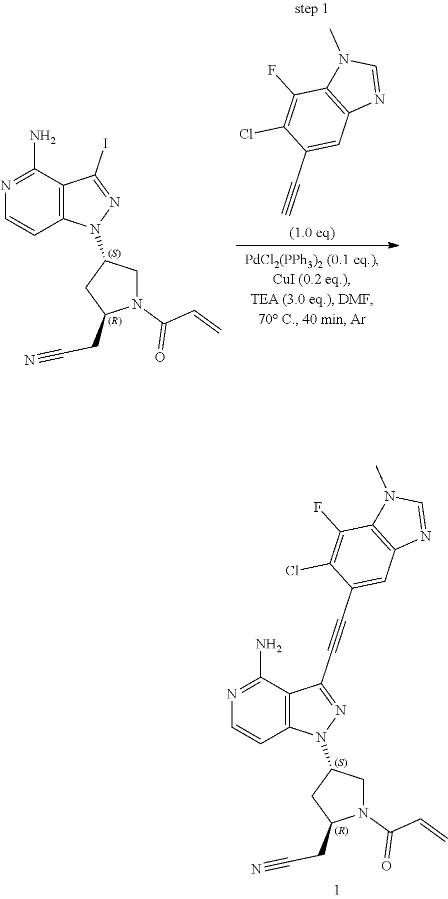

2-[(2R,4S)-4-{4-amino-3-[2-(6-chloro-7-fluoro-1-methyl-1,3-benzodiazol-5-yl)ethynyl]pyrazolo[4,3-c]pyridin-1-yl}-1-(prop-2-enoyl)pyrrolidin-2-yl]acetonitrile. MS ESI calculated for C₂₅H₂₀ClFN₈O [M+H]⁺, 503.14, 505.14, found 503.10, 505.10; ¹H NMR (400 MHz, CDCl₃) δ 7.95-7.84 (m, 3H), 6.73 (d, J=6.2 Hz, 1H), 6.46-6.39 (m, 2H), 5.83 (s, 2H), 5.77-5.74 (m, 1H), 5.53-5.50 (m, 1H), 4.77-4.74 (m, 1H), 4.27-4.22 (m, 1H), 4.20-4.13 (m, 1H), 4.06 (d, J=1.4 Hz, 3H), 3.45-3.40 (m, 1H), 3.00-2.95 (m, 1H), 2.74-2.69 (m, 1H), 2.54-2.47 (m, 1H).

### Example 175: 1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one

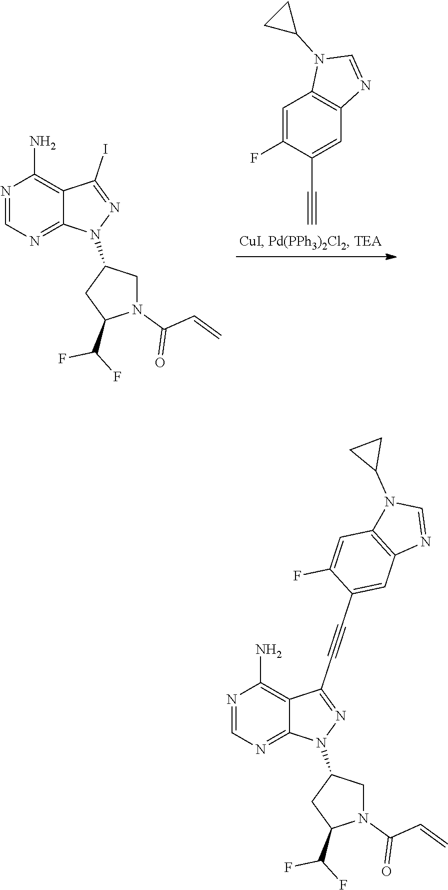 [SMILES: C=CC(=O)N1C[C@@H](n2nc(I)c3c(N)ncnc32)C[C@@H]1C(F)F ; C#Cc1cc2ncn(C3CC3)c2cc1F ; C=CC(=O)N1C[C@@H](n2nc(C#Cc3cc4ncn(C5CC5)c4cc3F)c3c(N)ncnc32)C[C@@H]1C(F)F]

1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one. ¹H NMR (400 MHz, DMSO-d₆): 8.37 (s, 1H), 8.30 (s, 1H), 8.15 (d, J=5.2 Hz, 1H), 7.67 (d, J=9.2 Hz, 1H), 6.74-6.51 (m, 1H), 6.21-6.11 (m, 1H), 5.76-5.58 (m, 2H), 4.63-4.46 (m, 1H), 4.17-3.92 (m, 2H), 3.55-3.50 (m, 1H), 2.76-2.57 (m, 1H), 2.26-2.11 (m, 1H), 1.30-1.38 (m, 3H), 1.14-1.03 (m, 4H). C₂₅H₂₂F₃N₇O [M+H] Calcd.: 507.2; Found, 507.2.

### Example 176: 1-((2R,4S)-4-(4-amino-3-((1-cyclopropyl-6-fluoro-1H-benzo[d]imidazol-5-yl)ethynyl)-1H-pyrazolo[4,3-c]pyridin-1-yl)-2-(difluoromethyl)pyrrolidin-1-yl)prop-2-en-1-one

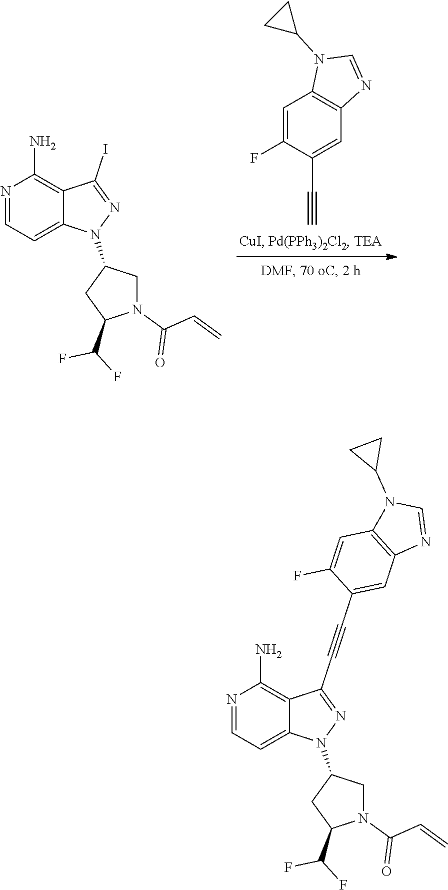 [SMILES: C=CC(=O)N1C[C@@H](n2nc(I)c3c(N)nccc32)C[C@@H]1C(F)F ; C#Cc1cc2ncn(C3CC3)c2cc1F ; C=CC(=O)N1C[C@@H](n2nc(C#Cc3cc4ncn(C5CC5)c4cc3F)c3c(N)nccc32)C[C@@H]1C(F)F]

1-((2R,4S)-4-(4-amino-3-((1-cyclopropyl-6-fluoro-1H-benzo[d]imidazol-5-yl)ethynyl)-1H-pyrazolo[4,3-c]pyridin-1-yl)-2-(difluoromethyl)pyrrolidin-1-yl)prop-2-en-1-one. ¹H NMR (400 MHz, DMSO-d₆): 8.37 (s, 1H), 8.06 (d, J=6.4 Hz, 1H), 7.80 (d, J=6.0 Hz, 1H), 7.67 (d, J=9.2 Hz, 1H), 7.04-7.02 (m, 1H), 6.73-6.47 (m, 3H), 6.21-6.12 (m, 2H), 5.74-5.63 (m, 1H), 5.55-5.50 (m, 1H), 4.55-4.38 (m, 1H), 4.18-3.79 (m, 2H), 3.56-3.51 (m, 1H), 2.78-2.58 (m, 1H), 2.25-2.08 (m, 1H), 1.32-1.30 (m, 3H), 1.13-1.06 (m, 4H). C₂₅H₂₃F₃N₆O [M+H] Calcd.: 506.2; Found, 506.1.

### Example 177: 1-((2R,4S)-4-(4-amino-3-((6-fluoro-1-methyl-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one

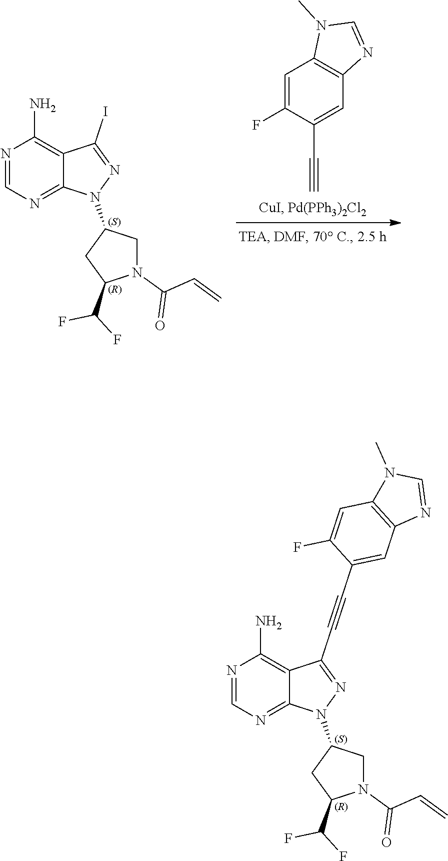

1-((2R,4S)-4-(4-amino-3-((6-fluoro-1-methyl-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one. ¹H NMR (400 MHz, DMSO-d₆): 8.33 (s, 1H), 8.30 (s, 1H), 8.14 (d, J=6.4 Hz, 1H), 7.70 (d, J=6.0 Hz, 1H), 6.80-6.54 (m, 2H), 6.80-6.19 (m, 2H), 5.76-5.73 (m, 1H), 5.62-5.58 (m, 1H), 4.98-4.68 (m, 1H), 4.16-4.04 (m, 2H), 3.84 (s, 3H), 2.79-2.54 (m, 2H). [M+H] Calcd.: 481.2; Found, 481.1.

### Example 178: 1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one

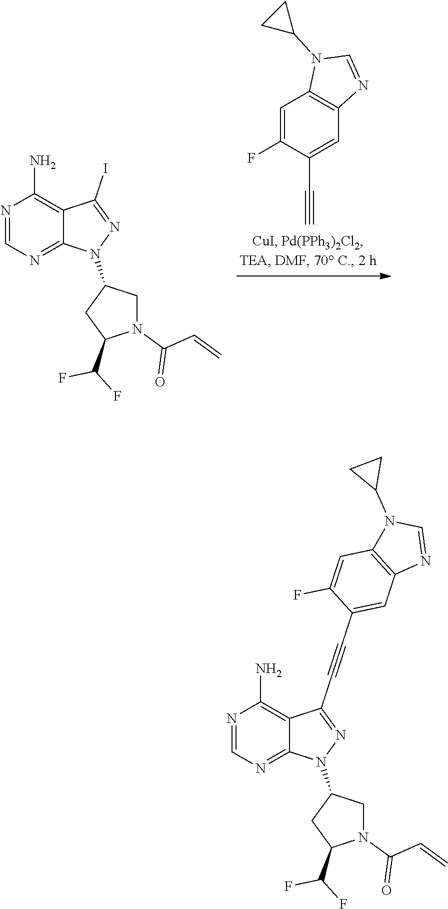 [SMILES: C=CC(=O)N1C[C@@H](n2nc(I)c3c(N)ncnc32)C[C@@H]1C(F)F ; C#Cc1cc2ncn(C3CC3)c2cc1F ; C=CC(=O)N1C[C@@H](n2nc(C#Cc3cc4ncn(C5CC5)c4cc3F)c3c(N)ncnc32)C[C@@H]1C(F)F]

1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one. ¹H NMR (400 MHz, DMSO-d₆): 8.37 (s, 1H), 8.30 (s, 1H), 8.15 (d, J=5.6 Hz, 1H), 7.67 (d, J=9.2 Hz, 1H), 6.80-6.20 (m, 4H), 5.76-5.73 (m, 1H), 5.63-5.58 (m, 1H), 4.75-4.68 (m, 1H), 4.15-4.05 (m, 2H), 3.55-3.50 (m, 1H), 2.67-2.57 (m, 2H), 1.14-1.03 (m, 4H). C₂₅H₂₂F₃N₇O [M+H] Calcd.: 507.2; Found, 507.1.

### Example 179: 1-((2R,4S)-4-(4-amino-3-((6-chloro-1-methyl-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one

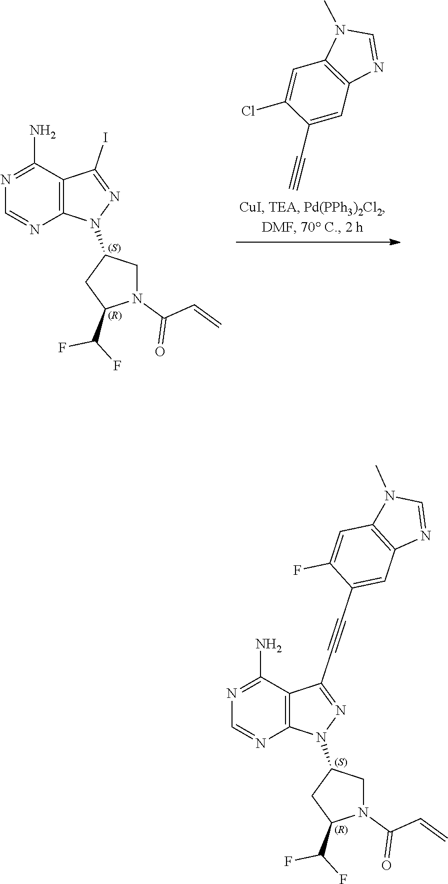

1-((2R,4S)-4-(4-amino-3-((6-chloro-1-methyl-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one. ¹H NMR (400 MHz, DMSO-d₆): 8.36 (s, 1H), 8.30 (s, 1H), 8.20 (s, 1H), 7.97 (s, 1H), 6.80-6.20 (m, 4H), 5.77-5.74 (m, 1H), 5.62-5.60 (m, 1H), 4.97-4.68 (m, 1H), 4.14-3.87 (m, 4H), 2.78-2.55 (m, 2H). [M+H] Calcd.: 497.1; Found, 497.1.

### Example 180: 1-((2R,4S)-4-(4-amino-3-((6-chloro-1-ethyl-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one

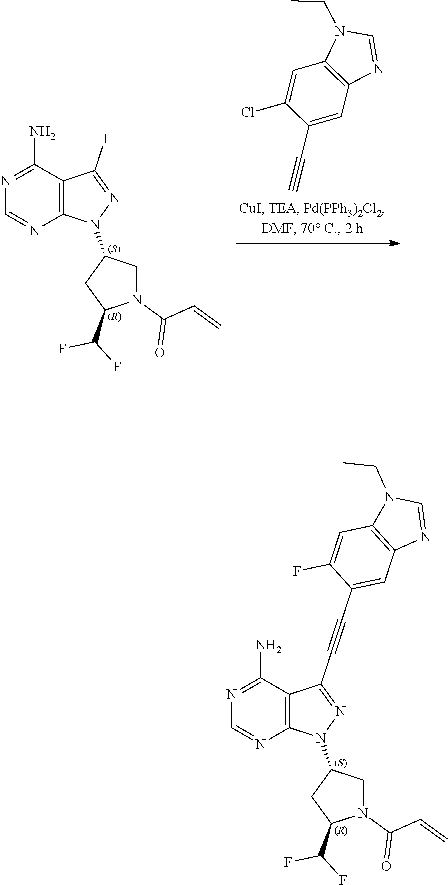

1-((2R,4S)-4-(4-amino-3-((1-ethyl-6-chloro-1H-benzo[d]imidazol-5-yl)ethynyl)-1H-pyrazolo[3,4-d]pyrimidin-1-yl)-2-(difluoromethyl)pyrrolidin-1-yl)prop-2-en-1-one. ¹H NMR (400 MHz, DMSO-d₆): 8.44 (s, 1H), 8.30 (s, 1H), 8.20 (s, 1H), 8.02 (s, 1H), 6.80-6.24 (m, 4H), 5.75 (d, J=10.0 Hz, 1H), 5.63-5.58 (m, 1H), 4.96-4.68 (m, 1H), 4.34-4.28 (m, 2H). 4.16-3.78 (m, 2H), 2.81-2.56 (m, 2H), 1.41 (t, J=7.2 Hz, 3H). [M+H] Calcd.: 511.1; Found, 511.2.

### Example 181: 1-((2R,4S)-4-(4-amino-3-((6-fluoro-1-methyl-1H-benzo[d]imidazol-5-yl)ethynyl)-1H-pyrazolo[4,3-c]pyridin-1-yl)-2-(difluoromethyl)pyrrolidin-1-yl)prop-2-en-1-one

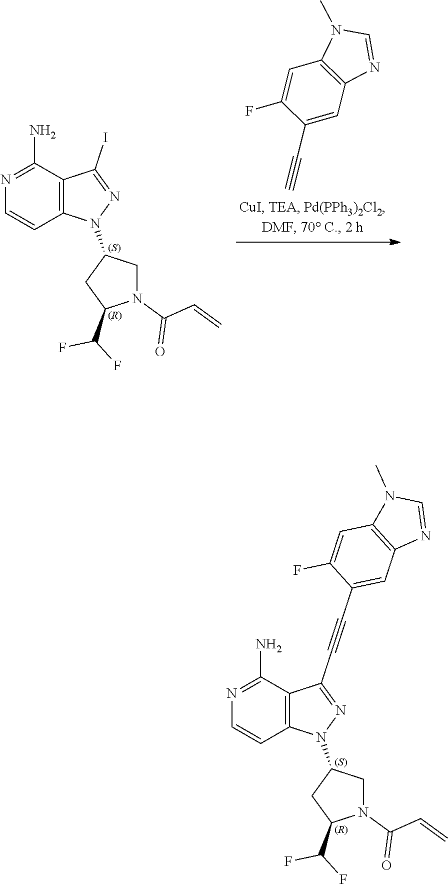

1-((2R,4S)-4-(4-amino-3-((6-fluoro-1-methyl-1H-benzo[d]imidazol-5-yl)ethynyl)-1H-pyrazolo[4,3-c]pyridin-1-yl)-2-(difluoromethyl)pyrrolidin-1-yl)prop-2-en-1-one. ¹H NMR (400 MHz, DMSO-d₆): 8.33 (s, 1H), 8.06-8.03 (m, 1H), 7.80 (d, J=6.0 Hz, 1H), 7.71 (d, J=10.0 Hz, 1H), 6.97 (d, J=6.0 Hz, 1H), 6.81-6.49 (m, 3H), 6.41-6.20 (m, 2H), 5.76-5.72 (m, 1H), 5.50-5.47 (m, 1H), 4.97-4.70 (m, 1H), 4.14-3.77 (m, 5H), 2.80-2.56 (m, 2H). [M+H] Calcd.: 480.2; Found, 480.2.

### Example 182: 1-((2R,4S)-4-(4-amino-3-((1-cyclopropyl-6-fluoro-1H-benzo[d]imidazol-5-yl)ethynyl)-1H-pyrazolo[4,3-c]pyridin-1-yl)-2-(difluoromethyl)pyrrolidin-1-yl)prop-2-en-1-one

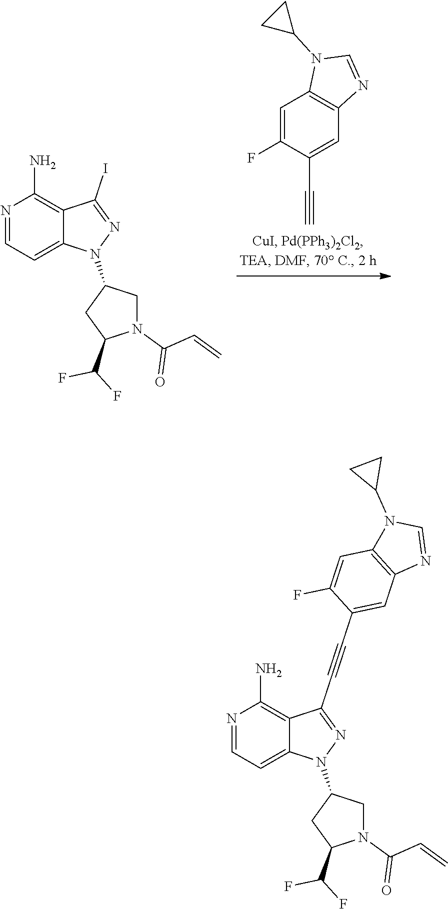 [SMILES: C=CC(=O)N1C[C@@H](n2nc(I)c3c(N)nccc32)C[C@@H]1C(F)F ; C#Cc1cc2ncn(C3CC3)c2cc1F ; C=CC(=O)N1C[C@@H](n2nc(C#Cc3cc4ncn(C5CC5)c4cc3F)c3c(N)nccc32)C[C@@H]1C(F)F]

1-((2R,4S)-4-(4-amino-3-((1-cyclopropyl-6-fluoro-1H-benzo[d]imidazol-5-yl)ethynyl)-1H-pyrazolo[4,3-c]pyridin-1-yl)-2-(difluoromethyl)pyrrolidin-1-yl)prop-2-en-1-one. ¹H NMR (400 MHz, DMSO-d₆): 8.39 (s, 1H), 8.13-7.86 (m, 2H), 7.67 (d, J=9.2 Hz, 1H), 6.70 (s, 1H), 6.81-6.55 (m, 3H), 6.41-6.13 (m, 2H), 5.77-5.73 (m, 1H), 5.50-5.45 (m, 1H), 4.96-4.68 (m, 1H), 4.13-3.76 (m, 2H), 3.55-3.50 (m, 1H), 2.80-2.56 (m, 2H), 1.13-1.09 (m, 4H). C₂₅H₂₃F₃N₆O [M+H] Calcd.: 506.2; Found, 506.1.

### Example 183: 1-((2R,4S)-4-(4-amino-3-((6-chloro-1-ethyl-1H-benzo[d]imidazol-5-yl)ethynyl)-1H-pyrazolo[4,3-c]pyridin-1-yl)-2-(difluoromethyl)pyrrolidin-1-yl)prop-2-en-1-one

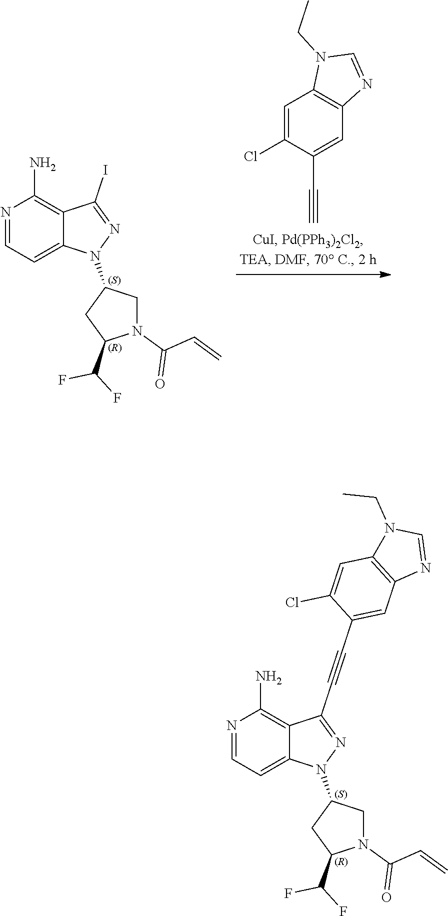

1-((2R,4S)-4-(4-amino-3-((6-chloro-1-ethyl-1H-benzo[d]imidazol-5-yl)ethynyl)-1H-pyrazolo[4,3-c]pyridin-1-yl)-2-(difluoromethyl)pyrrolidin-1-yl)prop-2-en-1-one. ¹H NMR (400 MHz, DMSO-d₆): 8.44 (s, 1H), 8.16 (s, 0.4H), 8.11 (s, 1H), 8.03 (s, 1H), 7.80 (d, J=6.0 Hz, 1H), 6.98 (d, J=6.0 Hz, 1H), 6.81-6.54 (m, 3H), 6.40-6.11 (m, 2H), 5.76-5.73 (m, 1H), 5.50-5.47 (m, 1H), 4.98-4.70 (m, 1H), 4.34-4.28 (m, 2H), 4.15-4.00 (m, 2H), 2.80-2.56 (m, 2H), 1.41 (t, J=7.2 Hz, 3H). [M+H] Calcd.: 510.2; Found, 510.2.

### Example 184: 1-[(2R,4S)-4-{4-Amino-3-[2-(6-fluoro-1-methyl-1,3-benzodiazol-5-yl)ethynyl]pyrazolo[3,4-d]pyrimidin-1-yl}-2-(fluoromethyl)pyrrolidin-1-yl]prop-2-en-1-one

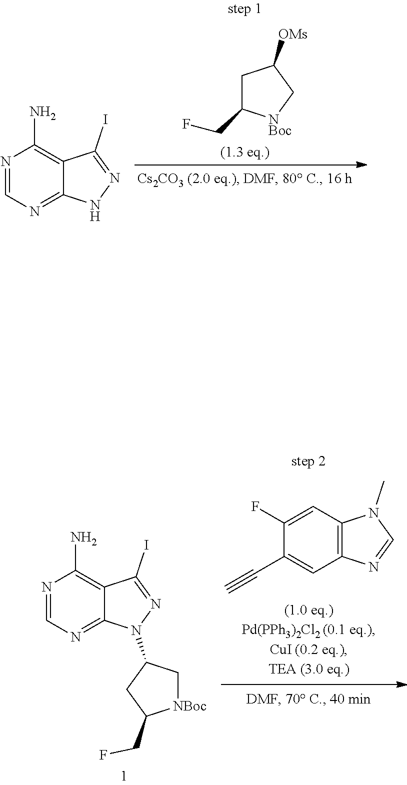 [SMILES: Nc1ncnc2[nH]nc(I)c12 ; CC(C)(C)OC(=O)N1C[C@H](OS(C)(=O)=O)C[C@@H]1CF ; CC(C)(C)OC(=O)N1C[C@@H](n2nc(I)c3c(N)ncnc32)C[C@@H]1CF ; C#Cc1cc2ncn(C)c2cc1F]

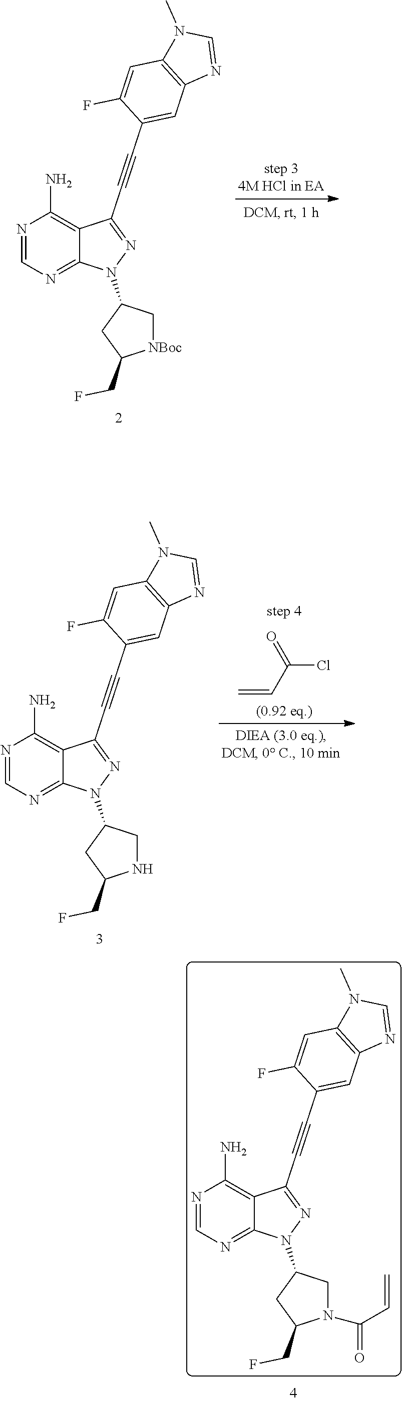

1-[(2R,4S)-4-{4-amino-3-[2-(6-fluoro-1-methyl-1,3-benzodiazol-5-yl)ethynyl]pyrazolo[3,4-d]pyrimidin-1-yl}-2-(fluoromethyl)pyrrolidin-1-yl]prop-2-en-1-one. MS ESI calculated for C₂₃H₂₀F₂N₈O [M+H]⁺, 463.17, found 463.25; ¹H NMR (300 MHz, DMSO-d₆) δ 8.38-8.28 (m, 2H), 8.17-7.92 (m, 1H), 7.72-7.42 (m, 1H), 6.62-6.42 (m, 1H), 6.21-5.96 (m, 1H), 5.84-5.48 (m, 2H), 4.92-4.44 (m, 3H), 4.09-3.94 (m, 2H), 3.87 (s, 3H), 2.83-2.58 (m, 1H), 2.50-2.41 (m, 1H).

### Example 185: 1-[(2R,4S)-4-{4-Amino-3-[2-(6-fluoro-1-methyl-1,3-benzodiazol-5-yl)ethynyl]pyrazolo[4,3-c]pyridin-1-yl}-2-(fluoromethyl)pyrrolidin-1-yl]prop-2-en-1-one

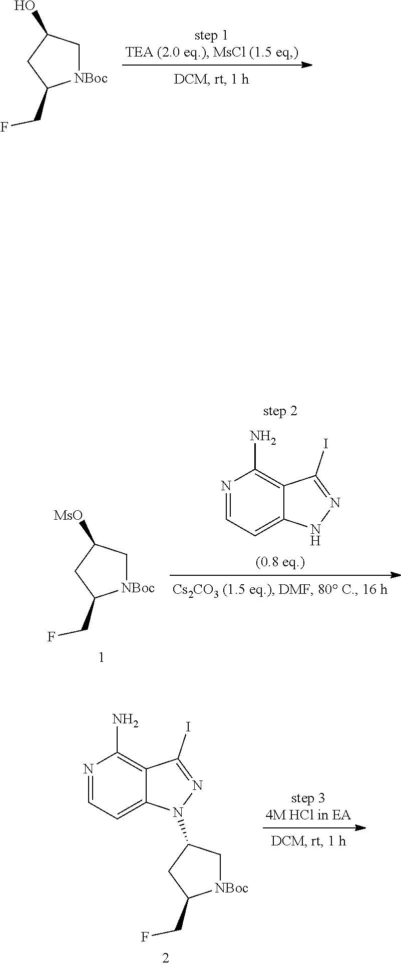 [SMILES: CC(C)(C)OC(=O)N1C[C@H](O)C[C@@H]1CF ; CC(C)(C)OC(=O)N1C[C@H](OS(C)(=O)=O)C[C@@H]1CF ; Nc1nccc2[nH]nc(I)c12 ; CC(C)(C)OC(=O)N1C[C@@H](n2nc(I)c3c(N)nccc32)C[C@@H]1CF]

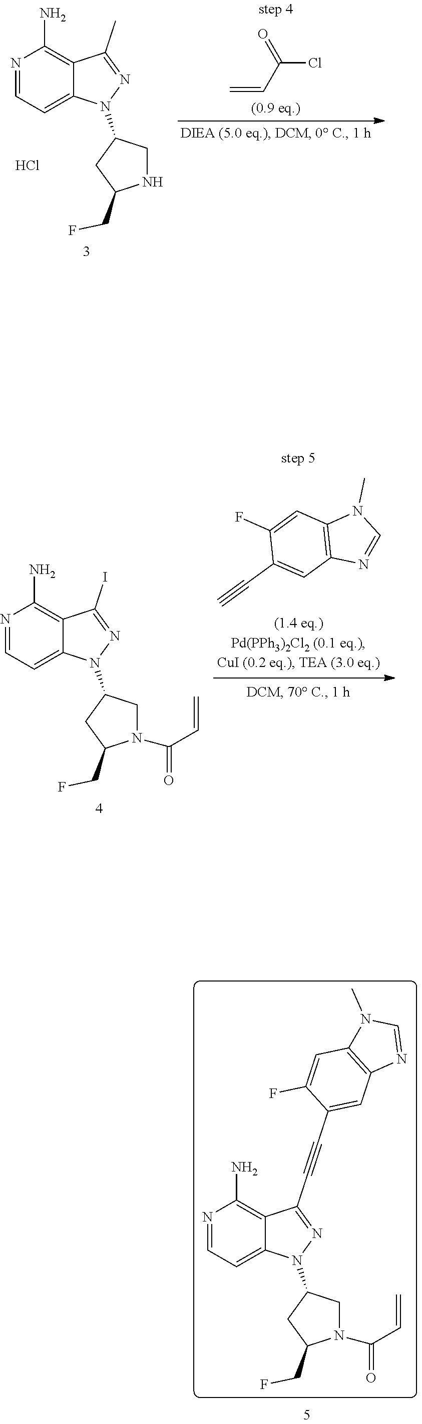

1-[(2R,4S)-4-{4-amino-3-[2-(6-fluoro-1-methyl-1,3-benzodiazol-5-yl)ethynyl]pyrazolo[4,3-c]pyridin-1-yl}-2-(fluoromethyl)pyrrolidin-1-yl]prop-2-en-1-one. MS ESI calculated for C₂₄H₂₁F₂N₇O [M+H]⁺, 462.18, found 462.15; ¹H NMR (400 MHz, DMSO-d₆) δ 8.33 (s, 1H), 8.06 (d, J=5.7 Hz, 1H), 7.86-7.51 (m, 3H), 7.08-6.38 (m, 4H), 6.19 (d, J=16.6 Hz, 1H), 5.71 (t, J=11.6 Hz, 1H), 5.58-5.38 (m, 1H), 4.88-4.44 (m, 3H), 4.12 (t, J=9.1 Hz, 1H), 3.86 (s, 3H), 2.68 (t, J=22.4 Hz, 1H), 2.33 (s, 1H).

### Example 186: 1-((2R,4S)-4-(4-Amino-5-((6-fluoro-1-methyl-1H-benzo[d]imidazol-5-yl)ethynyl)-7H-pyrrolo[2,3-d]pyrimidin-7-yl)-2-(fluoromethyl)pyrrolidin-1-yl)prop-2-en-1-one

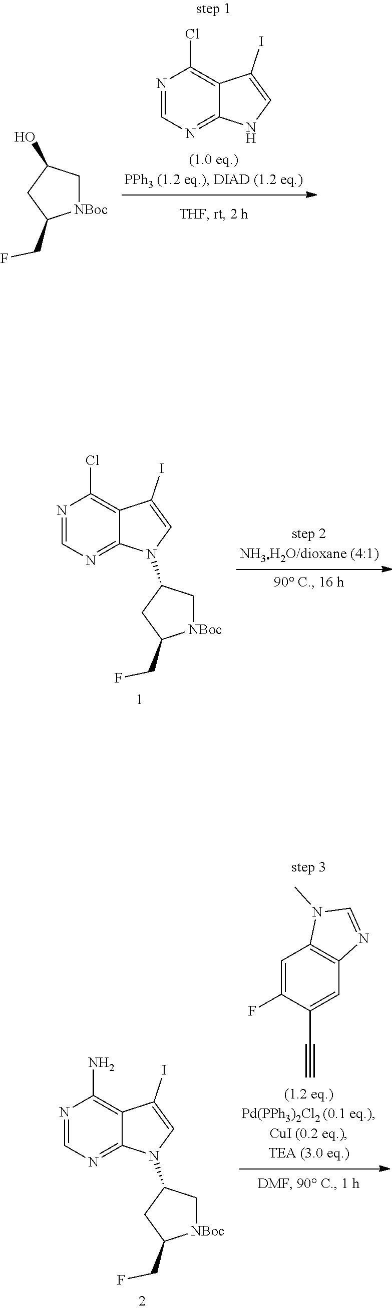 [SMILES: Clc1ncnc2[nH]cc(I)c12 ; CC(C)(C)OC(=O)N1C[C@H](O)C[C@@H]1CF ; CC(C)(C)OC(=O)N1C[C@@H](n2cc(I)c3c(Cl)ncnc32)C[C@@H]1CF ; CC(C)(C)OC(=O)N1C[C@@H](n2cc(I)c3c(N)ncnc32)C[C@@H]1CF ; C#Cc1cc2ncn(C)c2cc1F]

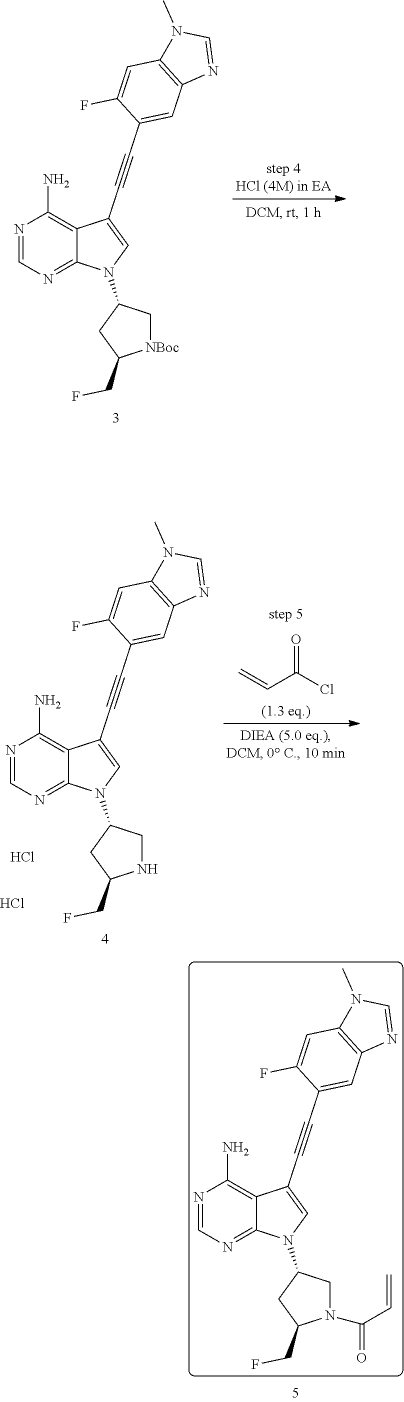

1-((2R,4S)-4-(4-amino-5-((6-fluoro-1-methyl-1H-benzo[d]imidazol-5-yl)ethynyl)-7H-pyrrolo[2,3-d]pyrimidin-7-yl)-2-(fluoromethyl)pyrrolidin-1-yl)prop-2-en-1-one. MS ESI calculated for C₂₄H₂₁F₂N₇O [M+H]⁺, 462.18, found 462.15; ¹H NMR (400 MHz, DMSO-d₆) δ 8.39-8.11 (m, 2H), 8.01-7.55 (m, 3H), 6.69-6.64 (m, 1H), 6.20-6.16 (m, 1H), 5.76-5.72 (m, 1H), 5.49-5.46 (m, 1H), 4.89-4.37 (m, 3H), 4.16-4.14 (m, 1H), 3.84 (s, 4H), 2.81-2.58 (m, 1H), 2.42 (d, J=7.2 Hz, 1H).

### Example 187: 2-[(2R,4S)-4-{4-Amino-3-[2-(6-chloro-1-cyclopropyl-1,3-benzodiazol-5-yl)ethynyl]pyrazolo[3,4-d]pyrimidin-1-yl}-1-(prop-2-enoyl)pyrrolidin-2-yl]acetonitrile

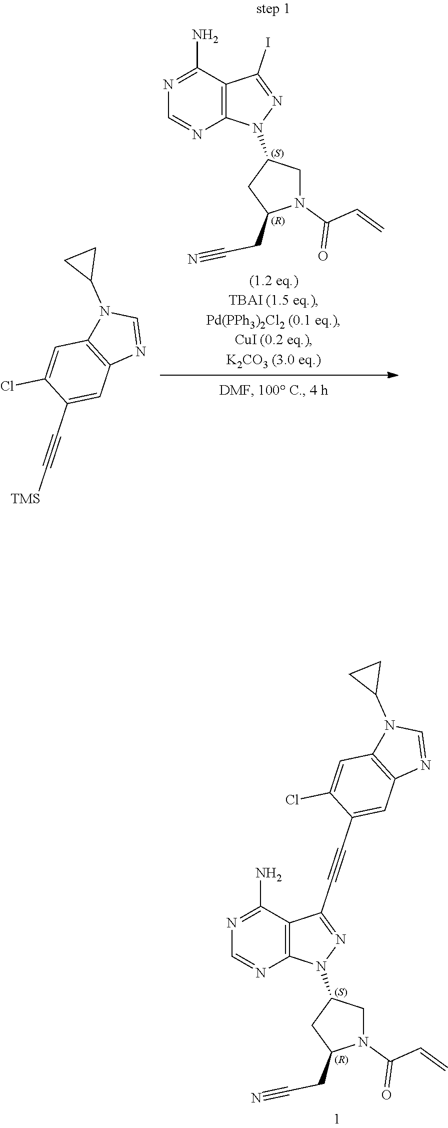

2-[(2R,4S)-4-{4-amino-3-[2-(6-chloro-1-cyclopropyl-1,3-benzodiazol-5-yl)ethynyl]pyrazolo[3,4-d]pyrimidin-1-yl}-1-(prop-2-enoyl)pyrrolidin-2-yl]acetonitrile. MS ESI calculated for C₂₆H₂₂ClN₉O [M+H]⁺, 512.16, found 512.15; ¹H NMR (400 MHz, CDCl₃) δ 8.39 (s, 1H), 8.08-7.96 (m, 2H), 7.71 (s, 1H), 6.47-6.39 (m, 2H), 6.26 (s, 2H), 5.75-5.46 (m, 2H), 4.79 (s, 1H), 4.2-4.17 (m, 2H), 3.52-3.24 (m, 2H), 3.05-2.96 (m, 1H), 2.86-2.65 (m, 1H), 2.56-2.13 (m, 1H), 1.25-1.16 (m, 2H), 1.14-1.06 (m, 2H).

### Example 188: 2-[(2R,4S)-4-{4-Amino-3-[2-(1-cyclopropyl-1,3-benzodiazol-5-yl)ethynyl]pyrazolo[3,4-d]pyrimidin-1-yl}-1-(prop-2-enoyl)pyrrolidin-2-yl]acetonitrile

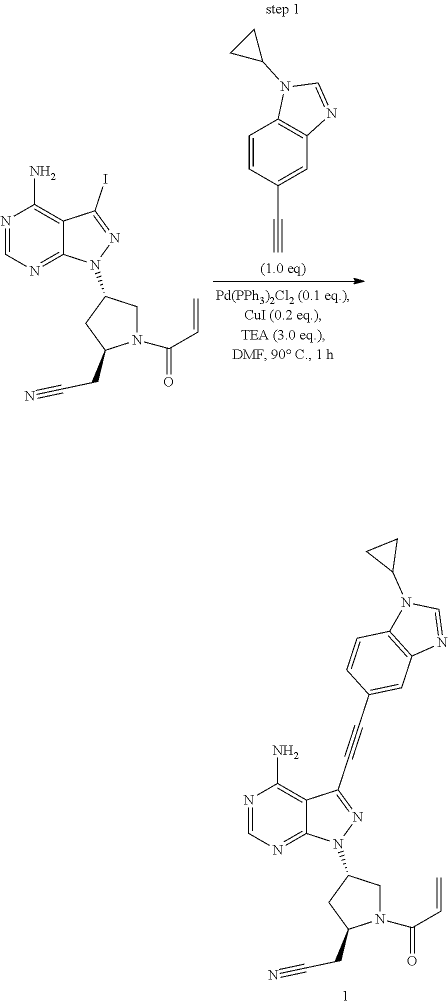 [SMILES: C=CC(=O)N1C[C@@H](n2nc(I)c3c(N)ncnc32)C[C@@H]1CC#N ; C#Cc1ccc2c(c1)ncn2C1CC1 ; C=CC(=O)N1C[C@@H](n2nc(C#Cc3ccc4c(c3)ncn4C3CC3)c3c(N)ncnc32)C[C@@H]1CC#N]

2-[(2R,4S)-4-{4-amino-3-[2-(1-cyclopropyl-1,3-benzodiazol-5-yl)ethynyl]pyrazolo[3,4-d]pyrimidin-1-yl}-1-(prop-2-enoyl)pyrrolidin-2-yl]acetonitrile. MS ESI calculated for C₂₆H₂₃N₉O [M+H]⁺, 478.20, found 478.15; ¹H NMR (400 MHz, CDCl₃) δ 8.40 (s, 1H), 8.06 (brs, 2H), 7.65-7.57 (m, 2H), 6.47-6.39 (m, 2H), 5.97 (s, 2H), 5.79-5.68 (m, 2H), 4.80-4.73 (m, 1H), 4.28-4.16 (m, 2H), 3.50-3.34 (m, 1H), 3.35-3.31 (m, 1H), 3.10-3.08 (m, 1H), 2.90-2.86 (m, 1H), 2.61-2.54 (m, 1H), 1.30-1.06 (m, 4H).

### Example 189: 1-[(3S)-3-{4-Amino-3-[2-(6,7-difluoro-1,2-dimethyl-1,3-benzodiazol-5-yl)ethynyl]pyrazolo[3,4-d]pyrimidin-1-yl}pyrrolidin-1-yl]prop-2-en-1-one

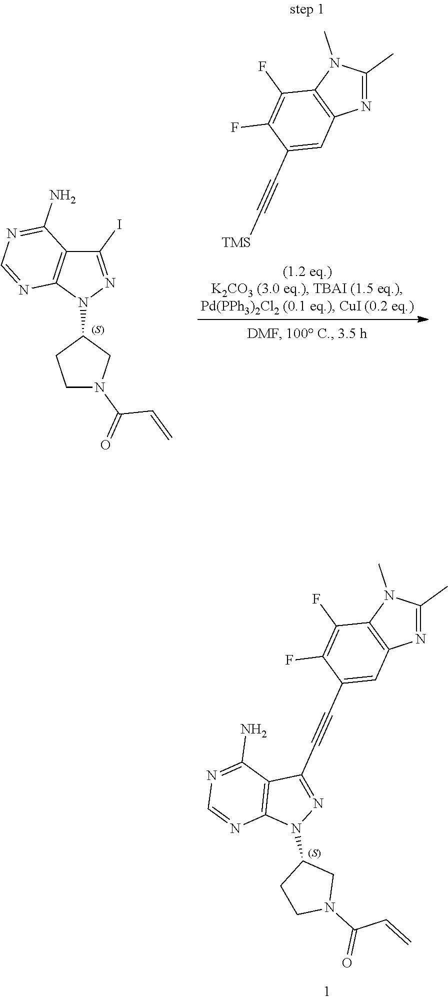

To a stirred solution of 1-[(3S)-3-{4-amino-3-iodopyrazolo[3,4-d]pyrimidin-1-yl}pyrrolidin-1-yl]prop-2-en-1-one (0.20 g, 0.52 mmol) in DMF (2.00 mL) were added 6,7-difluoro-1,2-dimethyl-5-[2-(trimethylsilyl)ethynyl]-1,3-benzodiazole (0.17 g, 0.63 mmol), K₂CO₃ (0.22 g, 1.56 mmol), TBAI (0.29 g, 0.78 mmol), CuI (19.83 mg, 0.10 mmol) and Pd(PPh₃)₂Cl₂ (36.54 mg, 0.05 mmol) at room temperature under nitrogen atmosphere. The reaction mixture was degassed with nitrogen for three times and stirred for 3.5 h at 100° C. The resulting mixture was concentrated under reduced pressure. The residue was purified by silica gel column chromatography, eluted with CH₂Cl₂/MeOH (10/1) to afford the crude product which was further purified by Prep-HPLC with the following conditions: Column: XBridge Prep C18 OBD Column, 19×150 mm 5 μm; Mobile phase A: water (10 mmol/L NH₄HCO₃), Mobile phase B: ACN; Flow rate: 20 mL/min; Gradient: 15 B to 50 B in 6 min; 210/254 nm; RT: 5.75 min. The fractions contained desired product were combined and concentrated to afford 1-[(3S)-3-{4-amino-3-[2-(6,7-difluoro-1,2-dimethyl-1,3-benzodiazol-5-yl)ethynyl]pyrazolo[3,4-d]pyrimidin-1-yl}pyrrolidin-1-yl]prop-2-en-1-one (42.30 mg, 17%) as a white solid. MS ESI calculated for C₂₃H₂₀F₂N₈O [M+H]⁺, 463.17, found 463.10; ¹H NMR (400 MHz, CDCl₃) δ 8.30 (s, 1H), 7.87-7.85 (m, 1H), 6.71-6.56 (m, 1H), 6.21-6.15 (m, 1H), 5.74-5.66 (m, 1H), 5.58-5.45 (m, 1H), 3.90-3.34 (m, 7H), 2.55 (s, 3H), 2.53-2.50 (m, 2H).

### Example 190: 1-[(3S)-3-{4-Amino-3-[2-(6-chloro-1-cyclopropyl-7-fluoro-1,3-benzodiazol-5-yl)ethynyl]pyrazolo[3,4-d]pyrimidin-1-yl}pyrrolidin-1-yl]prop-2-en-1-one

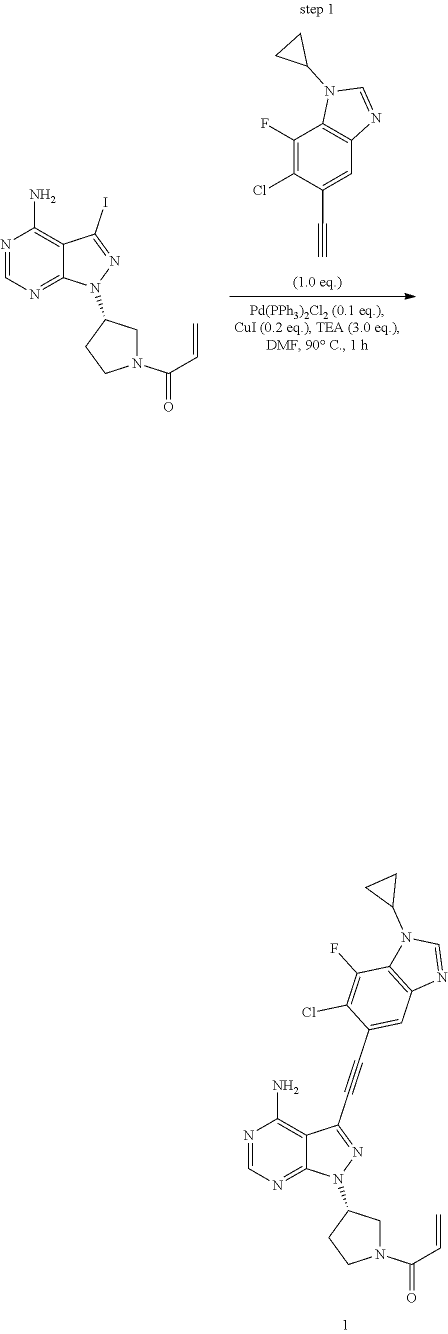 [SMILES: C=CC(=O)N1CC[C@H](n2nc(I)c3c(N)ncnc32)C1 ; C#Cc1cc2ncn(C3CC3)c2c(F)c1Cl ; C=CC(=O)N1CC[C@H](n2nc(C#Cc3cc4ncn(C5CC5)c4c(F)c3Cl)c3c(N)ncnc32)C1]

1-[(3S)-3-{4-amino-3-[2-(6-chloro-1-cyclopropyl-7-fluoro-1,3-benzodiazol-5-yl)ethynyl]pyrazolo[3,4-d]pyrimidin-1-yl}pyrrolidin-1-yl]prop-2-en-1-one. MS ESI calculated for C₂₄H₂₀ClFN₈O [M+H]⁺, 491.14, found 491.20; ¹H NMR (400 MHz, CDCl₃) δ 8.40 (d, J=2.9 Hz, 1H), 7.96 (d, J=13.2 Hz, 2H), 6.63-6.38 (m, 2H), 6.31 (brs, 1H), 5.82-5.50 (m, 2H), 4.19-3.93 (m, 3H), 3.80-3.75 (m, 1H), 3.64-3.61 (m, 1H), 2.60-2.55 (m, 2H), 1.36-1.22 (m, 4H).

### Example 191: 1-[(3S)-3-{4-Amino-3-[2-(6-chloro-1-cyclopropyl-1,3-benzodiazol-5-yl)ethynyl]pyrazolo[3,4-d]pyrimidin-1-yl}pyrrolidin-1-yl]prop-2-en-1-one

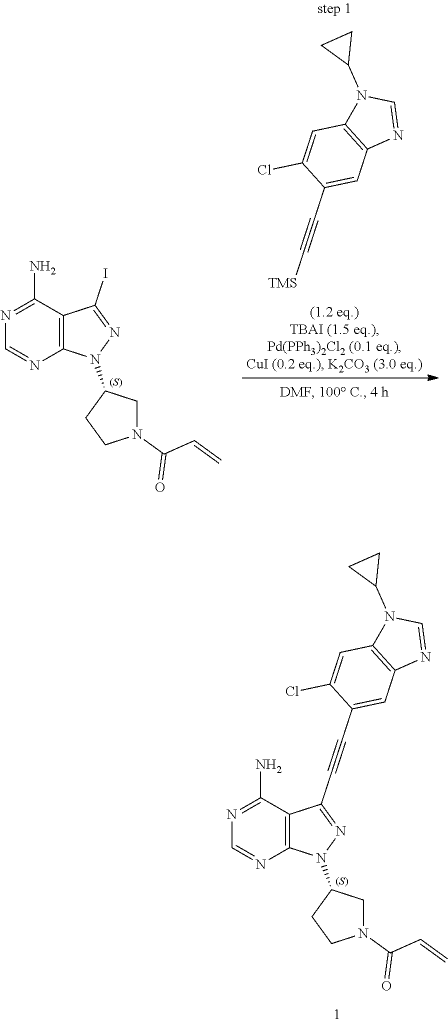

1-[(3S)-3-{4-amino-3-[2-(6-chloro-1-cyclopropyl-1,3-benzodiazol-5-yl)ethynyl]pyrazolo[3,4-d]pyrimidin-1-yl}pyrrolidin-1-yl]prop-2-en-1-one. MS ESI calculated for C₂₄H₂₁ClN₈O [M+H]⁺, 473.15, found 473.10; ¹H NMR (400 MHz, CDCl₃) δ 8.39 (s, 1H), 8.08-7.96 (m, 2H), 7.71 (s, 1H), 6.63-6.39 (m, 2H), 6.28 (brs, 2H), 5.86-5.47 (m, 2H), 4.19-3.98 (m, 3H), 3.88-3.71 (m, 1H), 3.41-3.21 (m, 1H), 2.83-2.37 (m, 2H), 1.30-1.06 (m, 4H).

### Example 192: 1-[(3S)-3-{4-Amino-3-[2-(6-fluoro-1,2-dimethyl-1,3-benzodiazol-5-yl)ethynyl]pyrazolo[3,4-d]pyrimidin-1-yl}pyrrolidin-1-yl]prop-2-en-1-one

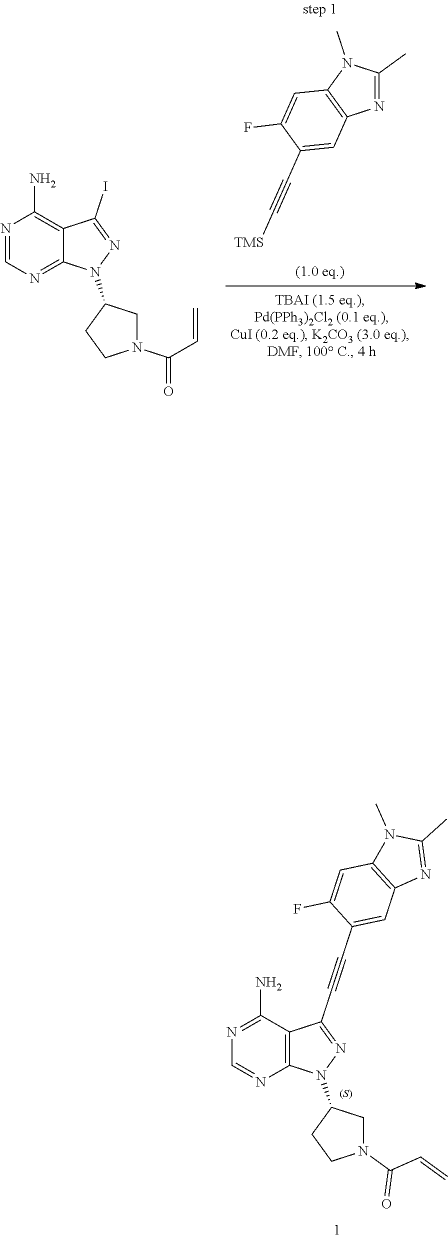

1-[(3S)-3-{4-amino-3-[2-(6-fluoro-1,2-dimethyl-1,3-benzodiazol-5-yl)ethynyl]pyrazolo[3,4-d]pyrimidin-1-yl}pyrrolidin-1-yl]prop-2-en-1-one. MS ESI calculated for C₂₃H₂₁FN₈O [M+H]⁺, 445.18, found 445.10; ¹H NMR (400 MHz, DMSO-d₆) δ 8.29 (s, 1H), 7.97 (dd, J=6.3, 2.9 Hz, 1H), 7.63 (d, J=9.9 Hz, 1H), 6.64-6.59 (m, 1H), 6.18-6.11 (m, 1H), 5.70-5.62 (m, 1H), 5.50-5.45 (m, 1H), 4.19-3.78 (m, 3H), 3.74 (s, 3H), 3.70-3.57 (m, 1H), 2.54 (s, 4H), 2.42-2.27 (m, 1H).

### Example 193: 1-[(3S)-3-{4-Amino-3-[2-(1-cyclopropyl-1,3-benzodiazol-5-yl)ethynyl]pyrazolo[3,4-d]pyrimidin-1-yl}pyrrolidin-1-yl]prop-2-en-1-one

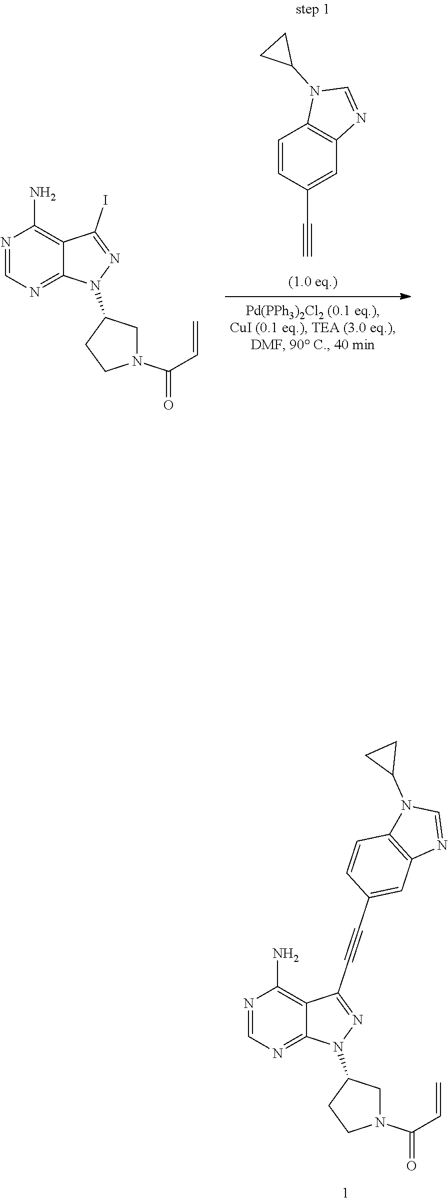 [SMILES: C=CC(=O)N1CC[C@H](n2nc(I)c3c(N)ncnc32)C1 ; C#Cc1ccc2c(c1)ncn2C1CC1 ; C=CC(=O)N1CC[C@H](n2nc(C#Cc3ccc4c(c3)ncn4C3CC3)c3c(N)ncnc32)C1]

1-[(3S)-3-{4-amino-3-[2-(1-cyclopropyl-1,3-benzodiazol-5-yl)ethynyl]pyrazolo[3,4-d]pyrimidin-1-yl}pyrrolidin-1-yl]prop-2-en-1-one. MS ESI calculated for C₂₄H₂₂N₈O [M+H]⁺, 439.19, found 439.10; ¹H NMR (400 MHz, CDCl₃) δ 8.40 (d, J=1.9 Hz, 1H), 8.06 (brs, 2H), 7.65-7.54 (m, 2H), 6.55-6.50 (m, 1H), 6.48-6.37 (m, 1H), 5.97-5.91 (m, 2H), 5.73-5.70 (m, 1H), 5.57-5.55 (m, 1H), 4.19-4.07 (m, 3H), 3.87-3.71 (m, 1H), 3.44-3.31 (m, 1H), 2.78-2.60 (m, 1H), 2.55-2.41 (m, 1H), 1.27-1.18 (m, 2H), 1.12-1.08 (m, 2H).

### Example 194: 1-[(3S)-3-{4-Amino-3-[2-(1-cyclopropyl-6,7-difluoro-1,3-benzodiazol-5-yl)ethynyl]pyrazolo[3,4-d]pyrimidin-1-yl}pyrrolidin-1-yl]prop-2-en-1-one

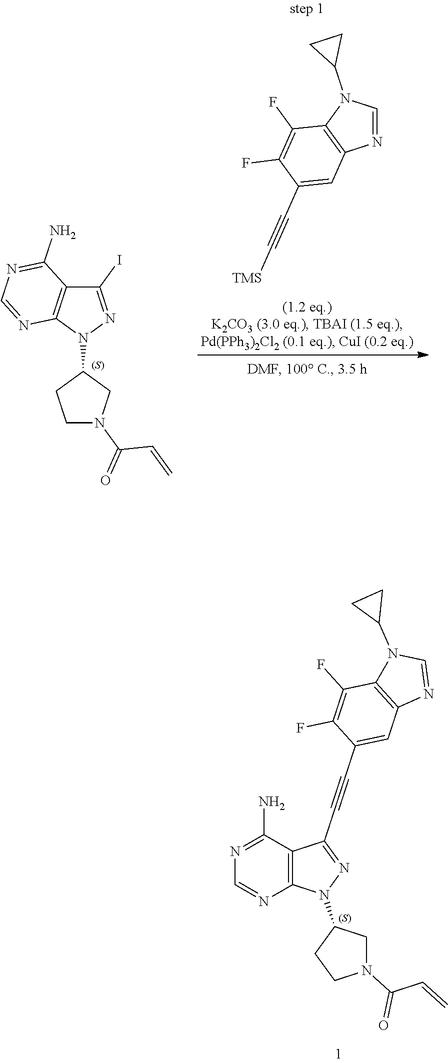

1-[(3S)-3-{4-amino-3-[2-(1-cyclopropyl-6,7-difluoro-1,3-benzodiazol-5-yl)ethynyl]pyrazolo[3,4-d]pyrimidin-1-yl}pyrrolidin-1-yl]prop-2-en-1-one. MS ESI calculated for C₂₄H₂₀F₂N₈O [M+H]⁺, 475.17, found 475.25; ¹H NMR (400 MHz, CDCl₃) δ 8.40 (d, J=4.6 Hz, 1H), 7.96 (d, J=4.2 Hz, 1H), 7.80-7.78 (m, 1H), 6.55-6.42 (m, 2H), 6.06 (s, 2H), 5.77-5.70 (m, 1H), 5.60-5.56 (m, 1H), 4.15-4.03 (m, 3H), 3.81-3.76 (m, 1H), 3.66-3.62 (m, 1H), 2.76-2.60 (m, 1H), 2.58-2.44 (m, 1H), 1.28-1.18 (m, 4H).

### Example 195: 1-[(3S)-3-{4-Amino-3-[2-(1-cyclopropyl-4,6-difluoro-1,3-benzodiazol-5-yl)ethynyl]pyrazolo[3,4-d]pyrimidin-1-yl}pyrrolidin-1-yl]prop-2-en-1-one

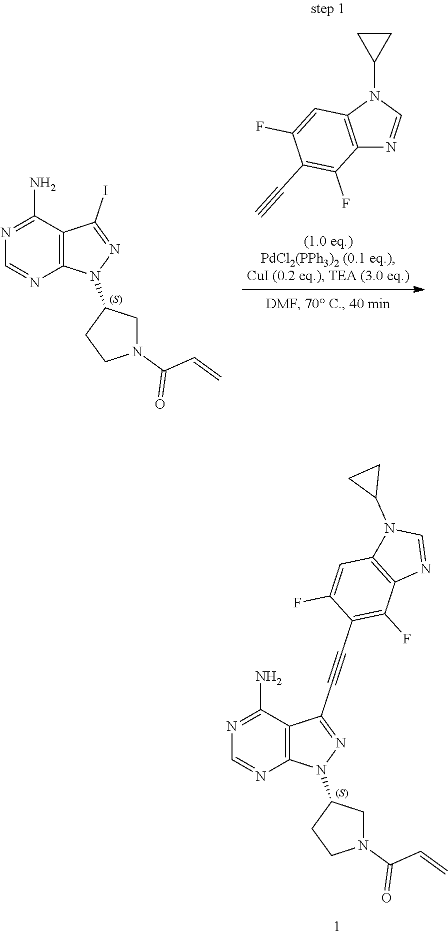

1-[(3S)-3-{4-amino-3-[2-(1-cyclopropyl-4,6-difluoro-1,3-benzodiazol-5-yl)ethynyl]pyrazolo[3,4-d]pyrimidin-1-yl}pyrrolidin-1-yl]prop-2-en-1-one. MS ESI calculated for C₂₄H₂₀F₂N₈O [M+H]⁺, 475.17, found 475.15; ¹H NMR (300 MHz, DMSO-d₆) δ 8.49-8.44 (s, 1H), 8.32-8.30 (d, J=0.9 Hz, 1H), 7.67-7.58 (m, 1H), 6.79-6.64 (m, 1H), 6.20-6.18 (m, 1H), 5.80-5.70 (m, 1H), 5.51-5.49 (s, 1H), 4.2-3.81 (m, 3H), 3.73-3.51 (m, 2H), 2.40-2.30 (m, 2H), 1.17-1.04 (m, 4H).

### Example 196: 1-((2R,4S)-4-(4-Amino-3-((1-cyclopropyl-6-fluoro-1H-benzo[d]imidazol-5-yl)ethynyl)-1H-pyrazolo[3,4-d]pyrimidin-1-yl)-2-((difluoromethoxy)methyl)pyrrolidin-1-yl)prop-2-en-1-one

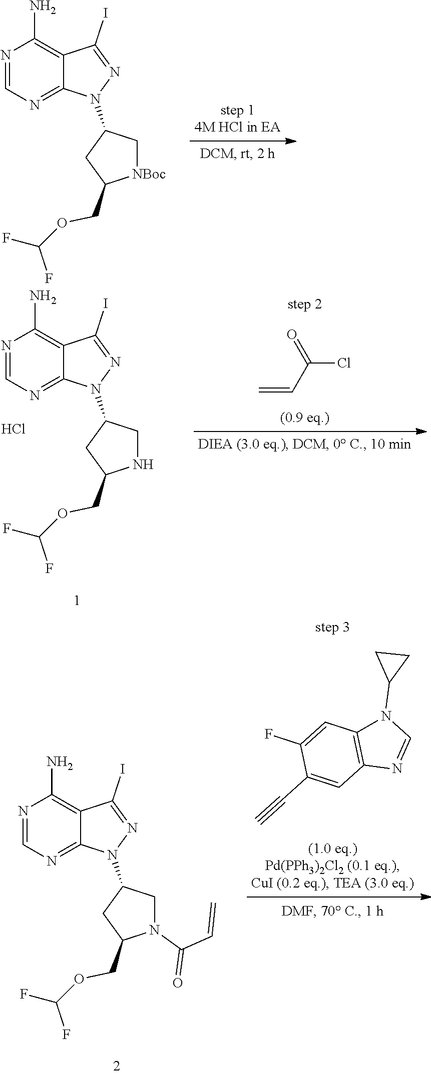

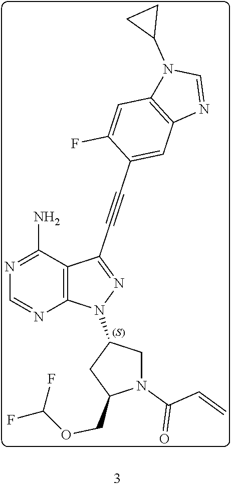

1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-((difluoromethoxy)methyl)pyrrolidin-1-yl)prop-2-en-1-one. MS ESI calculated for C₂₆H₂₃F₃N₈O₂ [M+H]⁺, 537.19, found 537.20; ¹H NMR (400 MHz, CDCl₃) δ 8.38 (d, J=5.2 Hz, 1H), 8.00 (d, J=5.8 Hz, 2H), 7.35 (d, J=9.1 Hz, 1H), 6.51-6.39 (m, 3H), 6.33-6.10 (m, 2H), 5.74-5.71 (m, 2H), 4.71 (d, J=39.9 Hz, 1H), 4.41 (dd, J=10.5, 4.1 Hz, 1H), 4.17-4.12 (m, 2H), 4.04 (dd, J=10.2, 2.8 Hz, 1H), 3.48-3.31 (m, 1H), 2.91 (q, J=9.3, 8.8 Hz, 1H), 2.54 (d, J=8.9 Hz, 1H), 1.32-1.17 (m, 2H), 1.09-1.00 (m, 2H).

### Example 197: 1-((2R,4S)-4-(4-Amino-3-((1-cyclopropyl-6-fluoro-1H-benzo[d]imidazol-5-yl)ethynyl)-1H-pyrazolo[3,4-d]pyrimidin-1-yl)-2-(fluoromethyl)pyrrolidin-1-yl)prop-2-en-1-one

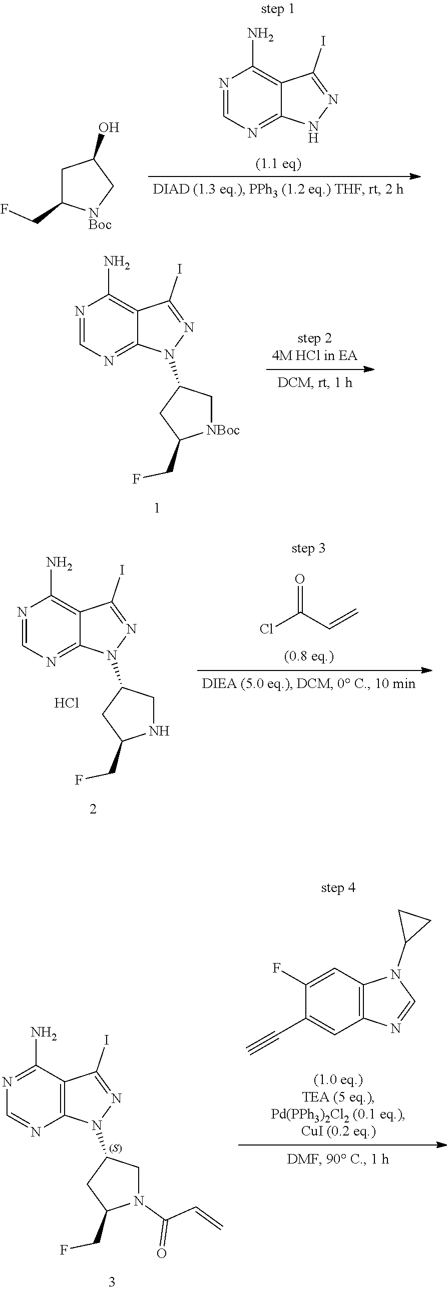

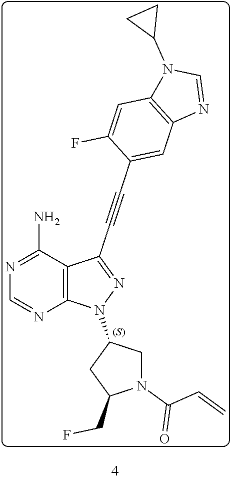

1-((2R,4S)-4-(4-amino-3-((1-cyclopropyl-6-fluoro-1H-benzo[d]imidazol-5-yl)ethynyl)-1H-pyrazolo[3,4-d]pyrimidin-1-yl)-2-(fluoromethyl)pyrrolidin-1-yl)prop-2-en-1-one. MS ESI calculated for C₂₅H₂₂F₂N₈O [M+H]⁺, 489.19, found 489.25; ¹H NMR (400 MHz, CDCl₃) δ 8.40 (s, 1H), 8.19-7.92 (m, 2H), 7.41 (s, 1H), 6.64-6.38 (m, 2H), 6.29 (s, 2H), 5.86-5.62 (m, 2H), 4.98-4.87 (m, 1H), 4.82-4.44 (m, 2H), 4.25-4.09 (m, 2H), 3.56-3.32 (m, 1H), 2.90 (d, J=12.6, 8.9 Hz, 1H), 2.66-2.45 (m, 1H), 1.19-1.15 (m, 4H).

### Example 198: 1-[(2R,4R)-4-{4-Amino-5-[2-(1-cyclopropyl-6-fluoro-1,3-benzodiazol-5-yl)ethynyl]imidazo[4,3-f][1,2,4]triazin-7-yl}-2-(methoxymethyl)pyrrolidin-1-yl]prop-2-en-1-one

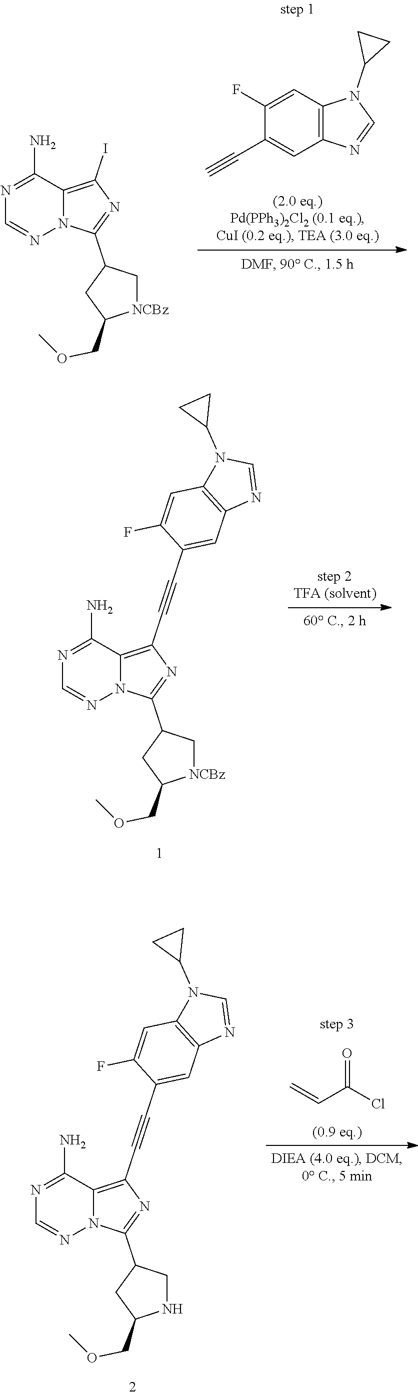

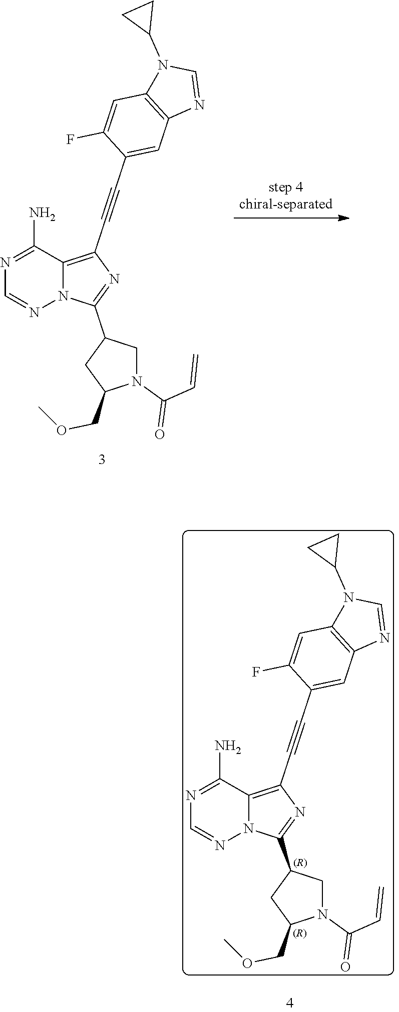

### Step 1: Benzyl (2R)-4-(4-Amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidine-1-carboxylate

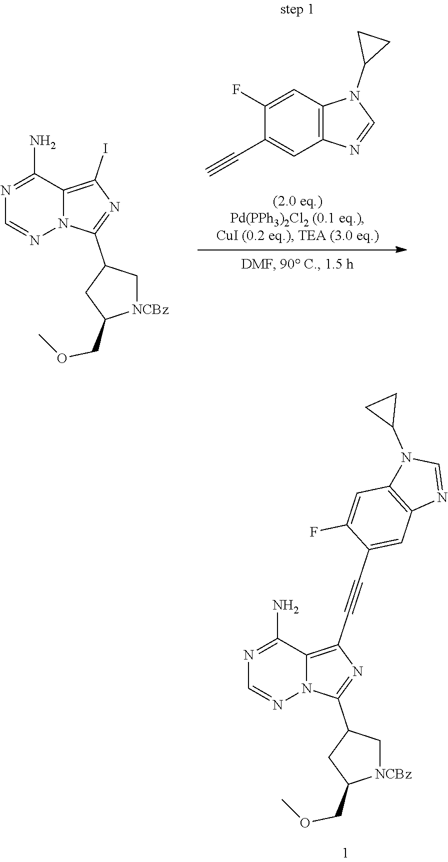

To a stirred mixture of benzyl (2R)-4-(4-amino-5-iodoimidazo[5,1-f][1,2,4]triazin-7-yl)-2-(methoxymethyl)pyrrolidine-1-carboxylate (1.00 g, 1.97 mmol), 1-cyclopropyl-5-ethynyl-6-fluoro-1,3-benzodiazole (0.79 g, 3.93 mmol), Pd(PPh₃)₂Cl₂ (0.14 g, 0.20 mmol) and CuI (74.86 mg, 0.39 mmol) in DMF (10.00 mL) was added TEA (0.82 mL, 5.90 mmol) dropwise at room temperature. The reaction mixture was degassed with nitrogen for three times and stirred for 1.5 h at 90° C. The resulting mixture was concentrated under reduced pressure. The residue was purified by silica gel column chromatography, eluted with 5% MeOH in DCM, the fractions contained desired product were combined and concentrated to afford benzyl (2R)-4-(4-amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidine-1-carboxylate (1.01 g, 88%) as a yellow solid. MS ESI calculated for C₃₁H₂₉FN₈O₃ [M+H]⁺, 581.23, found 581.15.

### Step 2: 5-((1-Cyclopropyl-6-fluoro-1H-benzo[d]imidazol-5-yl)ethynyl)-7-((5R)-5-(methoxymethyl)pyrrolidin-3-yl)imidazo[5,1-f][1,2,4]triazin-4-amine

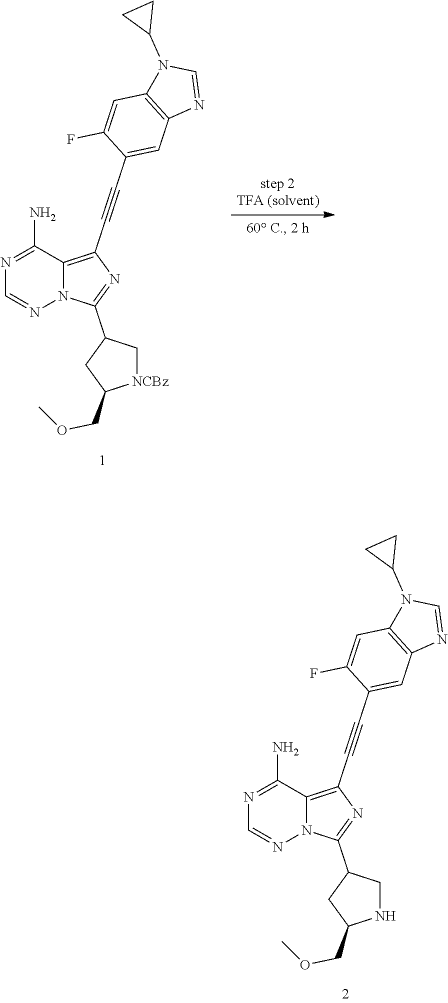

Into a 20 mL vial were added benzyl (2R)-4-(4-amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidine-1-carboxylate (1.01 g, 1.74 mmol) and TFA (6.06 mL) dropwise at room temperature. The reaction mixture was stirred for 2 h at 60° C. The reaction mixture was basified to pH 8 with saturated NaHCO₃ (aq.). The resulting mixture was concentrated under reduced pressure. The residue was purified by Prep-HPLC with the following conditions: Column: XBridge Prep C18 OBD Column, 19×150 mm 5 μm; Mobile phase A: water (10 mmol/L NH₄HCO₃), Mobile phase B: ACN; Flow rate: 20 mL/min; Gradient: 10 B to 65 B in 7 min; 210/254 nm; RT1: 6.50 min. The fractions contained desired product were combined and concentrated to afford 5-((1-cyclopropyl-6-fluoro-1H-benzo[d]imidazol-5-yl)ethynyl)-7-((5R)-5-(methoxymethyl)pyrrolidin-3-yl)imidazo[5,1-f][1,2,4]triazin-4-amine (0.61 g, 79%) as a light yellow solid. MS ESI calculated for C₂₃H₂₃FN₈O [M+H]⁺, 447.20, found 447.10; ¹H NMR (400 MHz, CDCl₃) δ 7.97 (d, J=5.2 Hz, 2H), 7.89 (s, 1H), 7.32 (d, J=9.1 Hz, 1H), 6.76 (s, 2H), 3.94-3.90 (m, 1H), 3.88 (s, 1H), 3.69 (s, 2H), 3.50 (s, 3H), 3.41-3.36 (m, 4H), 3.31-3.29 (m, 1H), 2.09-2.00 (m, 1H), 1.27-1.19 (m, 2H), 1.09-1.05 (m, 2H).

### Step 3: 1-((2R)-4-(4-Amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidin-1-yl)prop-2-en-1-one

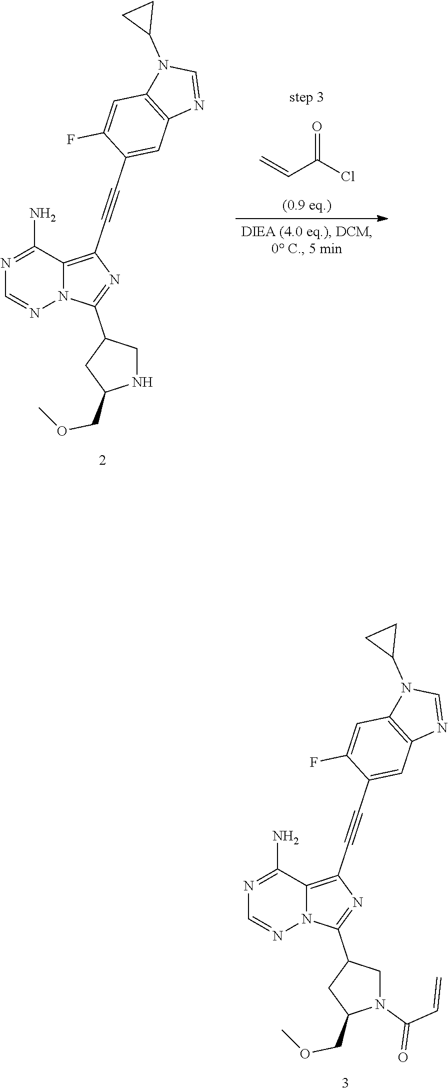

To a stirred solution of 5-((1-cyclopropyl-6-fluoro-1H-benzo[d]imidazol-5-yl)ethynyl)-7-((5R)-5-(methoxymethyl)pyrrolidin-3-yl)imidazo[5,1-f][1,2,4]triazin-4-amine (0.48 g, 1.09 mmol) in DCM (5.00 mL) were added DIEA (0.76 mL, 4.36 mmol) and acryloyl chloride (3.94 mL, 0.98 mmol) dropwise at 0° C. The reaction mixture was stirred for 5 min at 0° C. The reaction mixture was quenched with MeOH (5 mL) at 0° C. The resulting mixture was dissolved in water (50 mL) and extracted with EA (3×250 mL). The combined organic layers was washed with brine (2×50 mL), dried over anhydrous Na₂SO₄. The residue was purified by silica gel column chromatography, eluted with DCM/MeOH (10/1), the fractions contained desired product were combined and concentrated to afford 1-((2R)-4-(4-amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidin-1-yl)prop-2-en-1-one (0.45 g, 82%) as a white solid. MS ESI calculated for C₂₆H₂₅FN₈O₂ [M+H]⁺, 501.21, found 501.10.

### Step 4: 1-[(2R,4R)-4-{4-Amino-5-[2-(1-cyclopropyl-6-fluoro-1,3-benzodiazol-5-yl)ethynyl]imidazo[4,3-f][1,2,4]triazin-7-yl}-2-(methoxymethyl)pyrrolidin-1-yl]prop-2-en-1-one

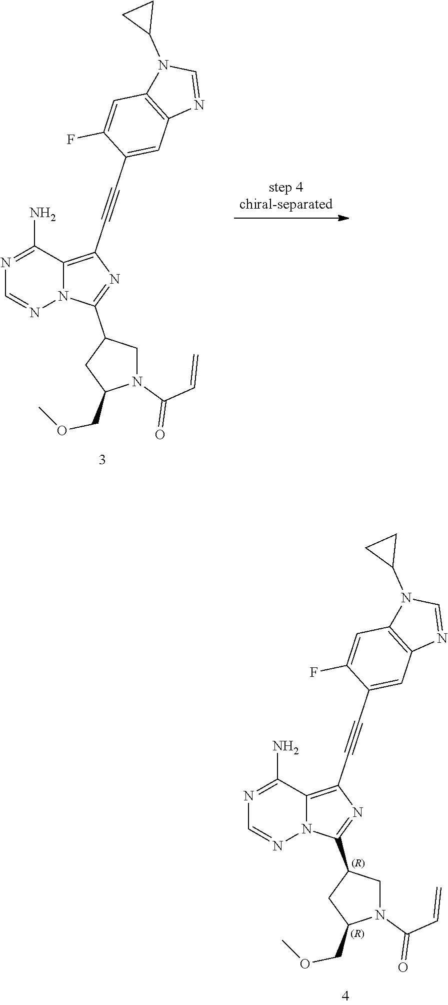

The crude product from last step (1-((2R)-4-(4-amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidin-1-yl)prop-2-en-1-one) was purified by Chiral-HPLC chromatography with the following conditions: Column: CHIRALPAK IF, 2×25 cm, 5 μm; Mobile phase A: Hex:DCM=1:1 (0.5% 2 M NH₃-MeOH)-HPLC, Mobile phase B: EtOH-HPLC; Flow rate: 16 mL/min; Gradient: 50% B to 50% B in 20 min; Wave length: 220/254 nm; RT1: 13.11 min. The fractions contained desired product were combined and concentrated to afford 1-[(2R,4R)-4-{4-amino-5-[2-(1-cyclopropyl-6-fluoro-1,3-benzodiazol-5-yl)ethynyl]imidazo[4,3-f][1,2,4]triazin-7-yl}-2-(methoxymethyl)pyrrolidin-1-yl]prop-2-en-1-one (96.30 mg, 21%) as a white solid. MS ESI calculated for C₂₆H₂₅FN₈O₂ [M+H]⁺, 501.21, found 501.15; ¹H NMR (400 MHz, CDCl₃) δ 8.74 (s, 1H), 8.35 (s, 1H), 8.09 (d, J=9.1 Hz, 1H), 8.01 (d, J=4 Hz, 1H), 7.64 (d, J=4 Hz, 1H), 6.87 (s, 1H), 6.75-6.58 (m, 1H), 6.20-6.14 (s, 1H), 5.70-5.67 (m, 1H), 4.48-4.36 (m, 1H), 4.23-4.05 (m, 2H), 3.91-3.84 (m, 1H), 3.55-3.45 (m, 2H), 3.40 (s, 1H), 2.43-2.25 (m, 1H), 1.14-1.04 (m, 4H).

### Example 199: 1-[(2R,4S)-4-{4-Amino-5-[2-(1-cyclopropyl-6-fluoro-1,3-benzodiazol-5-yl)ethynyl]imidazo[4,3-f][1,2,4]triazin-7-yl}-2-(methoxymethyl)pyrrolidin-1-yl]prop-2-en-1-one

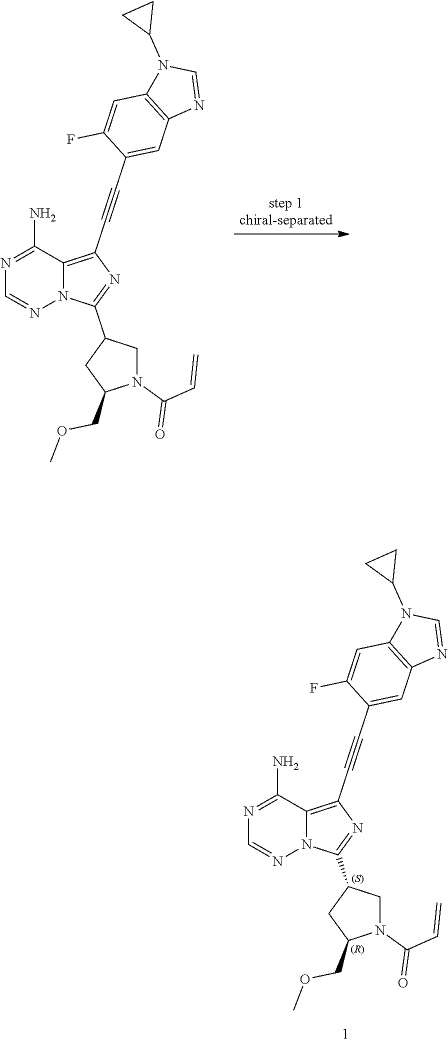

The crude product (1-((2R)-4-(4-amino-5-((1-cyclopropyl-6-fluoro-1H-benzo[d]imidazol-5-yl)ethynyl)imidazo[5,1-f][1,2,4]triazin-7-yl)-2-(methoxymethyl)pyrrolidin-1-yl)prop-2-en-1-one) was purified by Chiral-HPLC chromatography with the following conditions: Column: CHIRALPAK IF, 2×25 cm, 5 μm; Mobile phase A: Hex:DCM=1:1 (0.5% 2 M NH₃-MeOH)-HPLC, Mobile phase B: EtOH-HPLC; Flow rate: 16 mL/min; Gradient: 50% B to 50% B in 20 min; Wave length: 220/254 nm; RT2: 16.44 min. The fractions contained desired product were combined and concentrated to afford 1-[(2R,4S)-4-{4-amino-5-[2-(1-cyclopropyl-6-fluoro-1,3-benzodiazol-5-yl)ethynyl]imidazo[4,3-f][1,2,4]triazin-7-yl}-2-(methoxymethyl)pyrrolidin-1-yl]prop-2-en-1-one (0.16 g, 36%) as a white solid. MS ESI calculated for C₂₆H₂₅FN₈O₂ [M+H]⁺, 501.21, found 501.15. ¹H NMR (400 MHz, CDCl₃) δ 8.75 (s, 1H), 8.36 (s, 1H), 8.10 (d, J=9.1 Hz, 1H), 8.01 (s, 1H), 7.65 (d, J=4.1 Hz, 1H), 6.88 (s, 1H), 6.76-6.61 (m, 1H), 6.20-6.14 (m, 1H), 5.72-5.68 (m, 1H), 4.41-4.36 (m, 1H), 4.28-4.18 (m, 1H), 3.90-3.72 (m, 2H), 3.56-3.49 (m, 1H), 3.47-3.41 (m, 2H), 3.07 (s, 3H), 2.35-2.27 (m, 1H), 1.15-1.04 (m, 4H).

### Example 200: (S)-1-(3-(4-Amino-5-((1-cyclopropyl-6-fluoro-1H-benzo[d]imidazol-5-yl)ethynyl)imidazo[5,1-f][1,2,4]triazin-7-yl)pyrrolidin-1-yl)prop-2-en-1-one

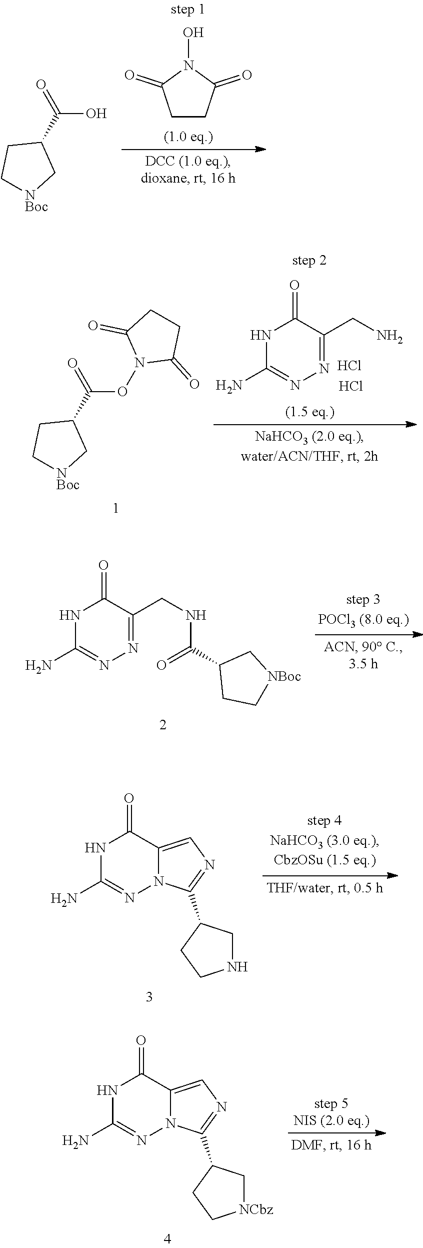

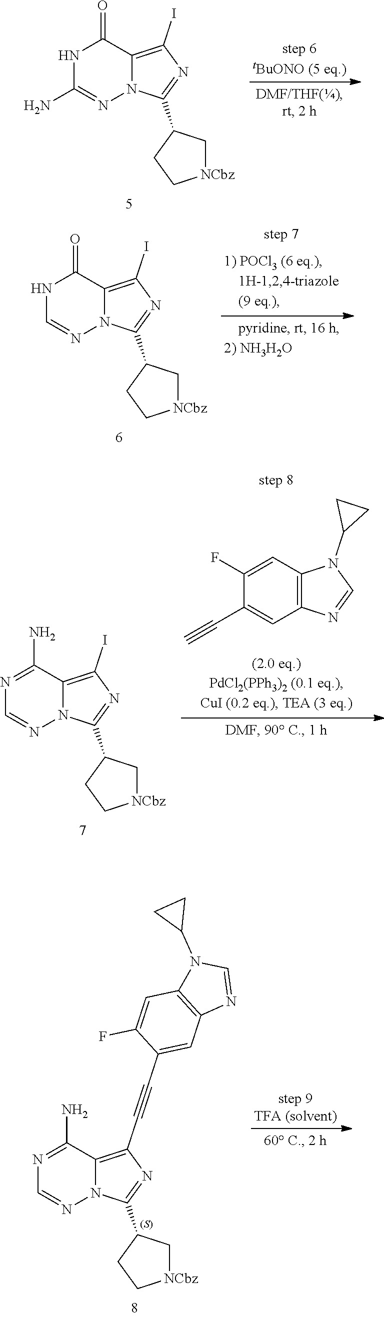

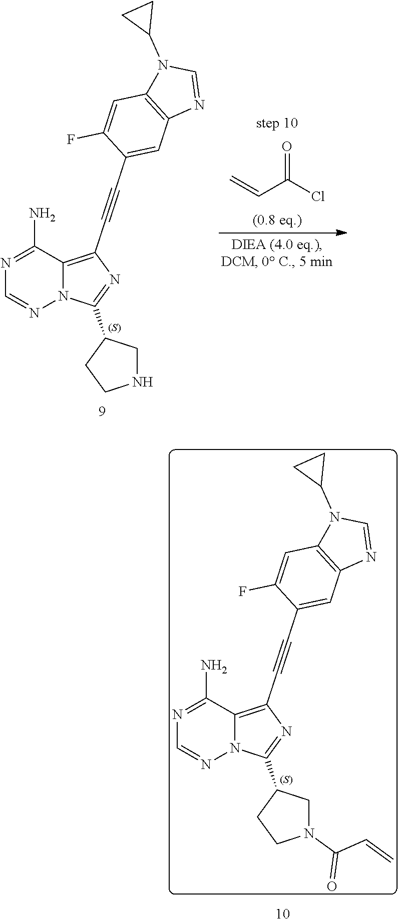

### Step 1: 1-(Tert-butyl) 3-(2,5-dioxopyrrolidin-1-yl) (S)-pyrrolidine-1,3-dicarboxylate

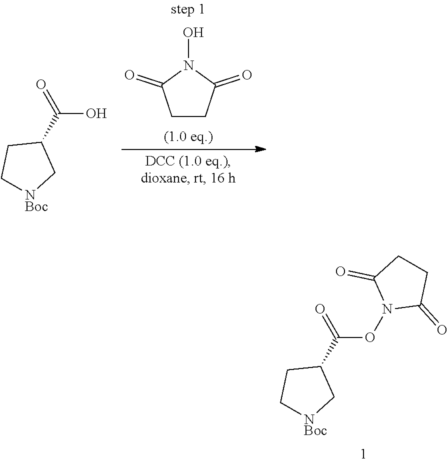 [SMILES: CC(C)(C)OC(=O)N1CC[C@H](C(=O)O)C1 ; O=C1CCC(=O)N1O ; CC(C)(C)OC(=O)N1CC[C@H](C(=O)ON2C(=O)CCC2=O)C1]

To a stirred mixture of (3S)-1-(tert-butoxycarbonyl)pyrrolidine-3-carboxylic acid (8.04 g, 37.35 mmol) in 1,4-dioxane (80.00 mL) was added N-hydroxysuccinimide (4.30 g, 37.35 mmol) dropwise at room temperature under nitrogen atmosphere. To the above mixture was added DCC (7.71 g, 37.35 mmol) dropwise at room temperature. The reaction mixture was stirred for 16 h at room temperature. The resulting mixture was filtered, the filter cake was washed with 1,4-dioxane (3×200 mL). The filtrate was concentrated under reduced pressure. The fractions contained desired product were combined and concentrated to afford 1-(tert-butyl) 3-(2,5-dioxopyrrolidin-1-yl) (S)-pyrrolidine-1,3-dicarboxylate (11.00 g, crude) as a yellow oil. MS ESI calculated for C₁₄H₂₀N₂O₆ [M+H−56]⁺, 257.13, found 257.05.

### Step 2: Tert-butyl (S)-3-(((3-Amino-5-oxo-4,5-dihydro-1,2,4-triazin-6-yl)methyl)carbamoyl)pyrrolidine-1-carboxylate

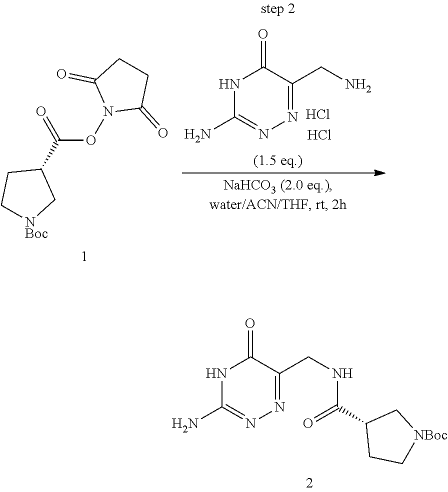

To a stirred solution of 3-amino-6-(aminomethyl)-4H-1,2,4-triazin-5-one dihydrochloride (8.02 g, 56.80 mmol) in H₂O (112.50 mL) was added NaHCO₃ (6.28 g, 74.73 mmol) in H₂O (56.30 mL) dropwise at 0° C. under air atmosphere. To the above mixture was added 1-(tert-butyl) 3-(2,5-dioxopyrrolidin-1-yl) (S)-pyrrolidine-1,3-dicarboxylate (11.67 g, 37.36 mmol) in ACN (46.69 mL) and THF (46.68 mL) dropwise at room temperature. The reaction mixture was stirred for 2 h at room temperature. The resulting mixture was concentrated under reduced pressure and purified by silica gel column chromatography, eluted with CH₂Cl₂/MeOH (8/1), the fractions contained desired product were combined and concentrated to afford tert-butyl (S)-3-(((3-amino-5-oxo-4,5-dihydro-1,2,4-triazin-6-yl)methyl)carbamoyl)pyrrolidine-1-carboxylate (4.60 g, 36%) as an off-white solid. MS ESI calculated for C₁₄H₂₂N₆O₄ [M+H]⁺, 339.17, found 339.20; ¹H NMR (400 MHz, DMSO-d₆) δ 12.04 (s, 1H), 8.13 (t, J=5.7 Hz, 1H), 6.79 (s, 2H), 4.08 (d, J=5.7 Hz, 2H), 3.42-3.39 (m, 1H), 3.25 (t, J=10.3 Hz, 1H), 3.18 (d, J=4.1 Hz, 2H), 2.98 (s, 1H), 2.07-1.82 (m, 2H), 1.40 (s, 9H).

### Step 3: (S)-2-Amino-7-(pyrrolidin-3-yl)imidazo[5,1-f][1,2,4]triazin-4 (3H)-one

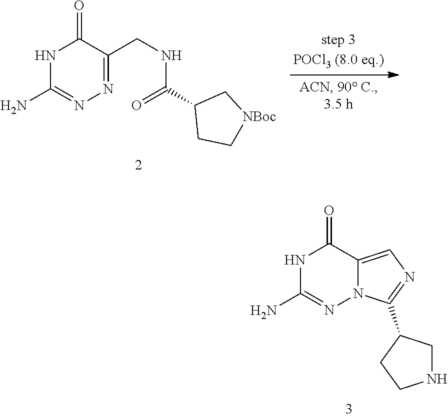 [SMILES: CC(C)(C)OC(=O)N1CC[C@H](C(=O)NCc2nnc(N)[nH]c2=O)C1 ; Nc1nn2c([C@H]3CCNC3)ncc2c(=O)[nH]1]

To a stirred solution of tert-butyl (S)-3-(((3-amino-5-oxo-4,5-dihydro-1,2,4-triazin-6-yl)methyl)carbamoyl)pyrrolidine-1-carboxylate (3.60 g, 10.64 mmol) in ACN (36.00 mL) was added POCl₃ (13.05 g, 85.12 mmol) dropwise at room temperature. The reaction mixture was degassed with nitrogen for three times and stirred for 3.5 h at 90° C. The resulting mixture was concentrated under reduced pressure. The residue was purified by reverse flash chromatography with the following conditions: Column: C18 silica gel; Mobile phase: MeCN in water (10 mmol NH₄HCO₃), 0% to 20% gradient in 20 min; Detector: UV 254 nm. The fractions contained desired product were combined and concentrated to afford (S)-2-amino-7-(pyrrolidin-3-yl)imidazo[5,1-f][1,2,4]triazin-4 (3H)-one (6.40 g, crude) as a white solid. MS ESI calculated for C₉H₁₂N₆O [M+H]⁺, 221.11, found 221.20.

### Step 4: Benzyl (S)-3-(2-Amino-4-oxo-3,4-dihydroimidazo[5,1-f][1,2,4]triazin-7-yl)pyrrolidine-1-carboxylate

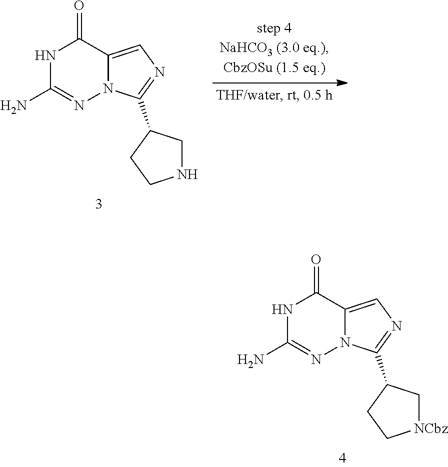 [SMILES: Nc1nn2c([C@H]3CCNC3)ncc2c(=O)[nH]1 ; Nc1nn2c([C@H]3CCN(C(=O)OCc4ccccc4)C3)ncc2c(=O)[nH]1]

To a stirred solution of (S)-2-amino-7-(pyrrolidin-3-yl)imidazo[5,1-f][1,2,4]triazin-4 (3H)-one (2.27 g, 10.31 mmol) in THF (20.00 mL) was added NaHCO₃ (2.60 g, 30.92 mmol) in water (20.00 mL) dropwise at 0° C. under air atmosphere. After 3 min, to the above mixture was added benzyl 2,5-dioxopyrrolidin-1-yl carbonate (3.85 g, 15.47 mmol) dropwise at room temperature. The reaction mixture was stirred for 0.5 h at room temperature. The resulting mixture was dissolved in water (30 mL) and extracted with EA (3×80 mL). The combined organic layers was washed with brine (2×60 mL), dried over anhydrous Na₂SO₄. After filtration, the filtrate was concentrated under reduced pressure. The residue was purified by silica gel column chromatography, eluted with CH₂Cl₂/MeOH (9/1), the fractions contained desired product were combined and concentrated to afford benzyl (S)-3-(2-amino-4-oxo-3,4-dihydroimidazo[5,1-f][1,2,4]triazin-7-yl)pyrrolidine-1-carboxylate (1.80 g, 49%) as an off-white solid. MS ESI calculated for C₁₇H₁₈N₆O₃ [M+H]⁺, 355.14, found 355.05; ¹H NMR (400 MHz, DMSO-d₆) δ 10.48 (s, 1H), 7.75 (d, J=8.3 Hz, 1H), 7.44-7.30 (m, 4H), 5.25-5.10 (m, 2H), 5.08-4.90 (m, 2H), 4.13-4.02 (m, 1H), 3.93-3.63 (m, 3H), 3.58-3.52 (m, 1H), 2.51-2.25 (m, 2H).

### Step 5: Benzyl (S)-3-(2-amino-5-iodo-4-oxo-3,4-dihydroimidazo[5,1-f][1,2,4]triazin-7-yl)pyrrolidine-1-carboxylate

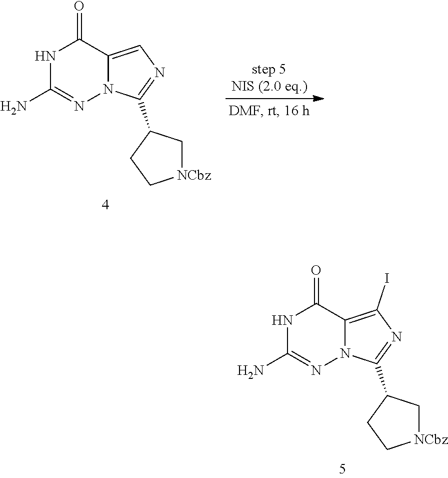 [SMILES: Nc1nn2c([C@H]3CCN(C(=O)OCc4ccccc4)C3)ncc2c(=O)[nH]1 ; Nc1nn2c([C@H]3CCN(C(=O)OCc4ccccc4)C3)nc(I)c2c(=O)[nH]1]

To a stirred solution of benzyl (S)-3-(2-amino-4-oxo-3,4-dihydroimidazo[5,1-f][1,2,4]triazin-7-yl)pyrrolidine-1-carboxylate (1.80 g, 5.08 mmol) in DMF (18.00 mL) was added NIS (2.29 g, 10.16 mmol) dropwise at room temperature. The reaction mixture was degassed with nitrogen for three times and stirred for 16 h at room temperature. The resulting mixture was dissolved in water (40 mL) and extracted with EA (3×80 mL). The combined organic layers was washed with brine (3×40 mL), dried over anhydrous Na₂SO₄. After filtration, the filtrate was concentrated under reduced pressure. The residue was purified by silica gel column chromatography, eluted with CH₂Cl₂/MeOH (10/1), the fractions contained desired product were combined and concentrated to afford benzyl (S)-3-(2-amino-5-iodo-4-oxo-3,4-dihydroimidazo[5,1-f][1,2,4]triazin-7-yl)pyrrolidine-1-carboxylate (3.10 g, 95%) as a brown oil. MS ESI calculated for C₁₇H₁₇IN₆O₃ [M+H]⁺, 481.04, found 481.00; ¹H NMR (400 MHz, DMSO-d₆) δ 10.31 (s, 1H), 7.40-7.28 (m, 4H), 5.12 (d, J=19.3 Hz, 4H), 4.06-3.50 (m, 5H), 2.39-2.32 (m, 3H).

### Step 6: Benzyl (S)-3-(5-iodo-4-oxo-3,4-dihydroimidazo[5,1-f][1,2,4]triazin-7-yl)pyrrolidine-1-carboxylate

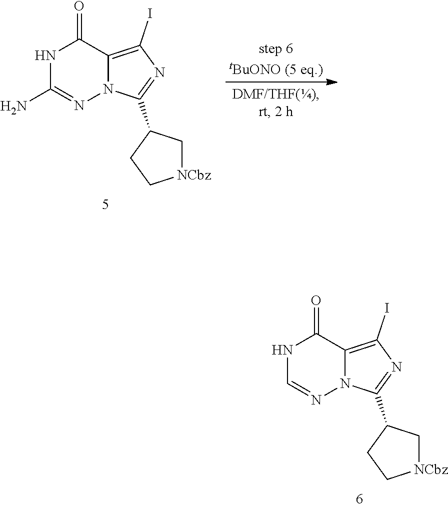 [SMILES: Nc1nn2c([C@H]3CCN(C(=O)OCc4ccccc4)C3)nc(I)c2c(=O)[nH]1 ; O=C(OCc1ccccc1)N1CC[C@H](c2nc(I)c3c(=O)[nH]cnn23)C1]

To a stirred solution of benzyl (S)-3-(2-amino-5-iodo-4-oxo-3,4-dihydroimidazo[5,1-f][1,2,4]triazin-7-yl)pyrrolidine-1-carboxylate (2.30 g, 4.79 mmol) in THF (120.00 mL) was added ^{t}BuONO (2.47 g, 23.94 mmol) in DMF (30.00 mL) dropwise at room temperature. The reaction mixture was degassed with nitrogen for three times and stirred for 2 h at room temperature. The resulting mixture was dissolved in water (50 mL) and extracted with EA (3×80 mL). The combined organic layers was washed with brine (4×40 mL), dried over anhydrous Na₂SO₄. After filtration, the filtrate was concentrated under reduced pressure. The residue was purified by silica gel column chromatography, eluted with CH₂Cl₂/MeOH (10/1), the fractions contained desired product were combined and concentrated to afford benzyl (S)-3-(5-iodo-4-oxo-3,4-dihydroimidazo[5,1-f][1,2,4]triazin-7-yl)pyrrolidine-1-carboxylate (1.76 g, 79%) as a brown oil. MS ESI calculated for C₁₇H₁₆IN₅O₃ [M+H]⁺, 466.03, found 466.05; ¹H NMR (400 MHz, DMSO-d₆) δ 10.32 (d, J=11.8 Hz, 1H), 7.55 (d, J=3.7 Hz, 1H), 7.37 (q, J=12.4, 8.8 Hz, 5H), 5.17 (s, 2H), 4.07-3.47 (m, 3H), 2.79 (s, 1H), 2.43 (d, J=46.4 Hz, 2H).

### Step 7: Benzyl (S)-3-(4-amino-5-iodoimidazo[5,1-f][1,2,4]triazin-7-yl)pyrrolidine-1-carboxylate

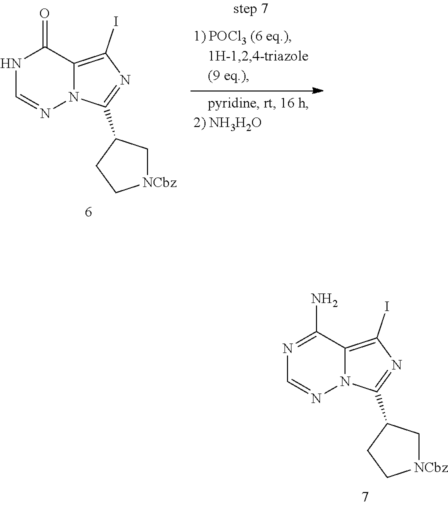 [SMILES: O=C(OCc1ccccc1)N1CC[C@H](c2nc(I)c3c(=O)[nH]cnn23)C1 ; Nc1ncnn2c([C@H]3CCN(C(=O)OCc4ccccc4)C3)nc(I)c12]

To a stirred solution of 1H-1,2,4-triazole (1.20 g, 17.37 mmol) in pyridine (30.00 mL) was added POCl₃ (1.78 g, 11.58 mmol) dropwise at room temperature. The reaction mixture was degassed with nitrogen for three times and stirred for 15 min at room temperature. To the above mixture was added benzyl (S)-3-(5-iodo-4-oxo-3,4-dihydroimidazo[5,1-f][1,2,4]triazin-7-yl)pyrrolidine-1-carboxylate (0.90 g, 1.93 mmol) in pyridine (12.00 mL) dropwise at room temperature. The reaction mixture was degassed with nitrogen for three times and stirred for another 16 h at room temperature. To a vigorously stirred solution of NH₃·H₂O (115.00 mL) was added the above reaction mixture dropwise at when the temperature stay below 0° C. The resulting mixture was extracted with EA (3×80 mL). The combined organic layers was washed with brine (2×40 mL), dried over anhydrous Na₂SO₄. After filtration, the filtrate was concentrated under reduced pressure. The residue was purified by silica gel column chromatography, eluted with CH₂Cl₂/MeOH (10/1), the fractions contained desired product were combined and concentrated to afford benzyl (S)-3-(4-amino-5-iodoimidazo[5,1-f][1,2,4]triazin-7-yl)pyrrolidine-1-carboxylate (0.48 g, 53%) as an off-white solid. MS ESI calculated for C₁₇H₁₇IN₆O₂ [M+H]⁺, 465.05, found 465.00; ¹H NMR (400 MHz, DMSO-d₆) δ 7.85 (s, 1H), 7.42-7.31 (m, 5H), 6.79-6.75 (m, 1H), 5.17 (s, 2H), 3.97 (d, J=12.0 Hz, 2H), 3.76 (t, J=8.3 Hz, 2H), 3.56 (t, J=9.1 Hz, 1H), 2.39 (s, 2H).

### Step 8: Benzyl (S)-3-(4-amino-5-((1-cyclopropyl-6-fluoro-1H-benzo[d]imidazol-5-yl)ethynyl)imidazo[5,1-f][1,2,4]triazin-7-yl)pyrrolidine-1-carboxylate

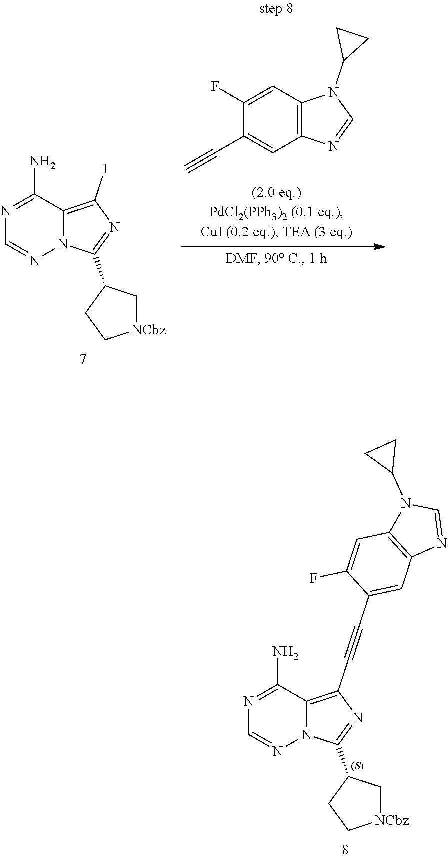

To a stirred mixture of benzyl (S)-3-(4-amino-5-iodoimidazo[5,1-f][1,2,4]triazin-7-yl)pyrrolidine-1-carboxylate (0.43 g, 0.93 mmol), 1-cyclopropyl-5-ethynyl-6-fluoro-1,3-benzodiazole (0.37 g, 1.86 mmol), Pd(PPh₃)₂Cl₂ (65.01 mg, 0.09 mmol) and CuI (35.28 mg, 0.18 mmol) in DMF (5.00 mL) was added TEA (0.28 g, 2.79 mmol) dropwise at room temperature. The reaction mixture was degassed with argon for three times and stirred for 1 h at 90° C. The resulting mixture was concentrated under reduced pressure. The residue was purified by silica gel column chromatography, eluted with CH₂Cl₂/MeOH (10/1), the fractions contained desired product were combined and concentrated to afford benzyl (S)-3-(4-amino-5-((1-cyclopropyl-6-fluoro-1H-benzo[d]imidazol-5-yl)ethynyl)imidazo[5,1-f][1,2,4]triazin-7-yl)pyrrolidine-1-carboxylate (0.49 g, 99%) as a white solid. MS ESI calculated for C₂₉H₂₅FN₈O₂ [M+H]⁺, 537.21, found 537.40.

### Step 9: (S)-5-((1-Cyclopropyl-6-fluoro-1H-benzo[d]imidazol-5-yl)ethynyl)-7-(pyrrolidin-3-yl)imidazo[5,1-f][1,2,4]triazin-4-amine

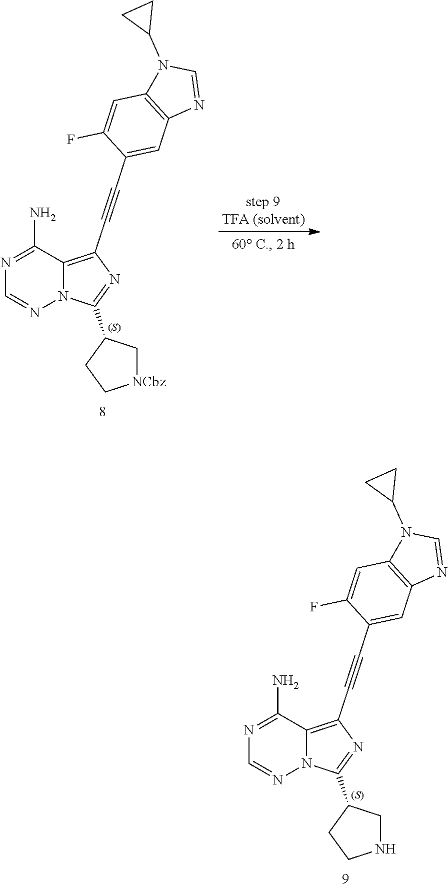

Into a 100 mL bottle were added benzyl (S)-3-(4-amino-5-((1-cyclopropyl-6-fluoro-1H-benzo[d]imidazol-5-yl)ethynyl)imidazo[5,1-f][1,2,4]triazin-7-yl)pyrrolidine-1-carboxylate (0.49 g, 0.92 mmol) and TFA (15.00 mL) dropwise at room temperature. The reaction mixture was stirred for 2 h at 60° C. under air atmosphere. The resulting mixture was concentrated under reduced pressure. The residue was basified to pH 8 with saturated NaHCO₃ (aq.). The resulting mixture was purified by reverse flash chromatography with the following conditions: Column: C18 silica gel; Mobile phase: ACN in water (10 mmol NH₄HCO₃), 95% in 20 min; detector: UV 254 nm. The fractions contained desired product were combined and concentrated to afford (S)-5-((1-cyclopropyl-6-fluoro-1H-benzo[d]imidazol-5-yl)ethynyl)-7-(pyrrolidin-3-yl)imidazo[5,1-f][1,2,4]triazin-4-amine (0.30 g, 81%) as a brown solid. MS ESI calculated for C₂₁H₁₉FN₈ [M+H]⁺, 403.17, found 403.10.

### Step 10: (S)-1-(3-(4-Amino-5-((1-cyclopropyl-6-fluoro-1H-benzo[d]imidazol-5-yl)ethynyl)imidazo[5,1-f][1,2,4]triazin-7-yl)pyrrolidin-1-yl)prop-2-en-1-one

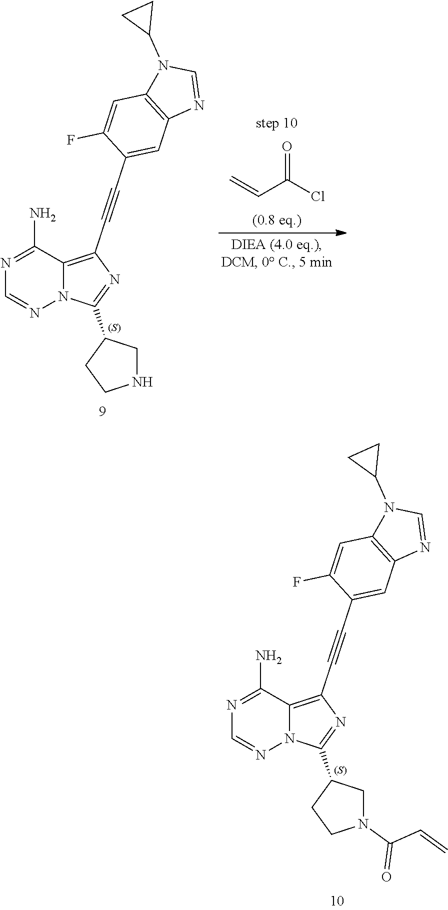

To a stirred solution of (S)-5-((1-cyclopropyl-6-fluoro-1H-benzo[d]imidazol-5-yl)ethynyl)-7-(pyrrolidin-3-yl)imidazo[5,1-f][1,2,4]triazin-4-amine (0.25 g, 0.63 mmol) in DCM (5.00 mL) were added DIEA (0.32 g, 2.52 mmol) and acryloyl chloride (45.52 mg, 0.50 mmol) dropwise at 0° C. The reaction mixture was stirred for 5 min at 0° C. under air atmosphere. The resulting mixture was dissolved in water (15 mL) and extracted with CH₂Cl₂ (2×40 mL). The combined organic layers was washed with brine (2×10 mL), dried over anhydrous Na₂SO₄. After filtration, the filtrate was concentrated under reduced pressure. The residue was purified by silica gel column chromatography, eluted with CH₂Cl₂/MeOH (10/1) to afford the crude product which was further purified by Prep-HPLC with the following conditions: Column: XBridge Shield RP18 OBD Column, 30×150 mm, 5 μm; Mobile phase A: water (10 mmol/L NH₄HCO₃), Mobile phase B: ACN; Flow rate: 60 mL/min; Gradient: 20% B to 40% B in 8 min; wave length: 254 nm; RT: 7.2 min. The fractions contained desired product were combined and concentrated to afford (S)-1-(3-(4-amino-5-((1-cyclopropyl-6-fluoro-1H-benzo[d]imidazol-5-yl)ethynyl)imidazo[5,1-f][1,2,4]triazin-7-yl)pyrrolidin-1-yl)prop-2-en-1-one (50.70 mg, 18%) as a white solid. MS ESI calculated for C₂₄H₂₁FN₈O [M+H]⁺, 457.18, found 457.20; ¹H NMR (400 MHz, DMSO-d₆) δ 8.77 (s, 1H), 8.35 (s, 1H), 8.08 (d, J=6.3 Hz, 1H), 8.01 (s, 1H), 7.64 (d, J=9.5 Hz, 1H), 6.87 (s, 1H), 6.57-6.69 (m, 1H), 6.09-6.33 (m, 1H), 5.54-5.64 (m, 1H), 4.14-3.84 (m, 3H), 3.83-3.58 (m, 2H), 3.48-3.57 (m, 1H), 2.47-2.12 (m, 2H), 1.32-1.16 (m, 4H).

### Example 201: 1-[(3 S)-3-{8-Amino-1-[2-(1-cyclopropyl-6-fluoro-1,3-benzodiazol-5-yl)ethynyl]imidazo[1,5-a]pyrazin-3-yl}pyrrolidin-1-yl]prop-2-en-1-one

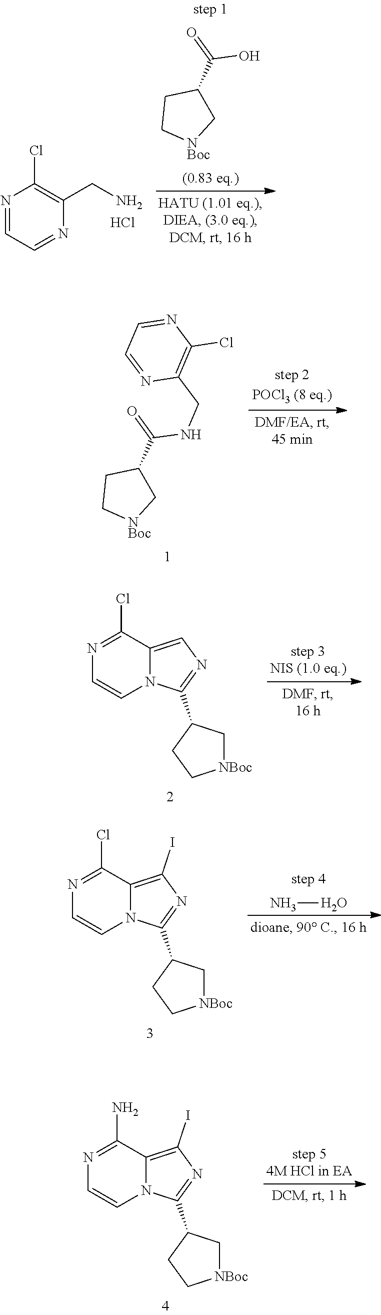

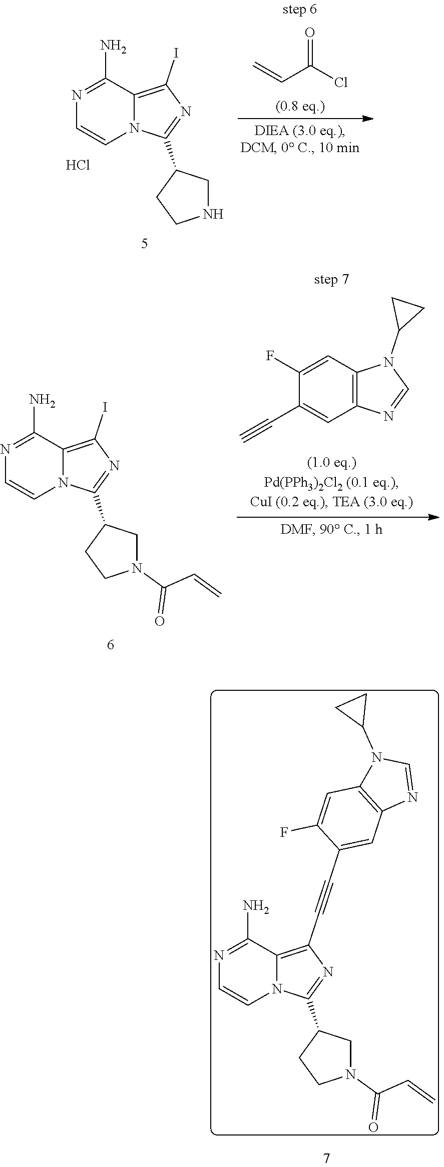

### Step 1: Tert-butyl (3S)-3-{[(3-chloropyrazin-2-yl)methyl]carbamoyl}pyrrolidine-1-carboxylate

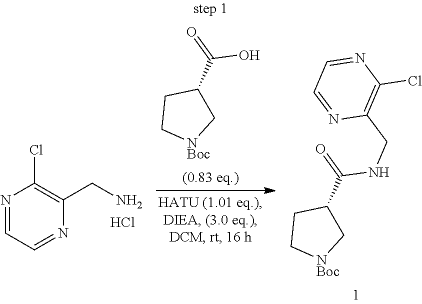 [SMILES: NCc1nccnc1Cl ; Cl ; CC(C)(C)OC(=O)N1CC[C@H](C(=O)O)C1 ; CC(C)(C)OC(=O)N1CC[C@H](C(=O)NCc2nccnc2Cl)C1]

To a stirred mixture of 1-(3-chloropyrazin-2-yl)methanamine hydrochloride (10.00 g, 55.54 mmol), DIEA (23.69 g, 0.17 mol), HATU (21.33 g, 56.10 mmol) in DCM (600.00 mL) was added (3R)-1-(tert-butoxycarbonyl)pyrrolidine-3-carboxylic acid (10.00 g, 46.45 mmol) in portions at room temperature. The reaction mixture was degassed with nitrogen for three times and stirred for 16 h at room temperature. The resulting mixture was quenched by water (100 mL) and extracted with DCM (3×150 mL). The combined organic layers was washed with brine (2×100 mL), dried over anhydrous Na₂SO₄ and filtered. The filtrate was concentrated under reduced pressure. The residue was purified by silica gel column chromatography, eluted with DCM/MeOH (10/1). The fractions contained desired product were combined and concentrated to afford tert-butyl (3S)-3-{[(3-chloropyrazin-2-yl)methyl]carbamoyl}pyrrolidine-1-carboxylate (21.70 g, crude) as a light yellow oil. MS ESI calculated for C₁₅H₂₁CLN₄O₃ [M+H−56]⁺, 285.13, found 285.20.

### Step 2: Tert-butyl (3S)-3-{8-chloroimidazo[1,5-a]pyrazin-3-yl}pyrrolidine-1-carboxylate

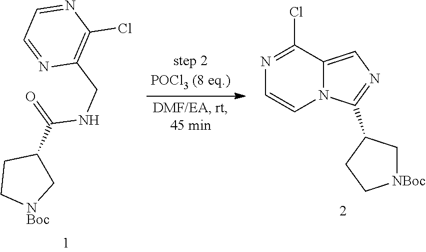 [SMILES: CC(C)(C)OC(=O)N1CC[C@H](C(=O)NCc2nccnc2Cl)C1 ; CC(C)(C)OC(=O)N1CC[C@H](c2ncc3c(Cl)nccn23)C1]

To a stirred solution of tert-butyl (3S)-3-{[(3-chloropyrazin-2-yl)methyl]carbamoyl}pyrrolidine-1-carboxylate (20.00 g, 58.68 mmol) in DMF (20.00 mL) and EA (400.00 mL) was added POCl₃ (71.98 g, 0.47 mol) dropwise at 0° C. The reaction mixture was degassed with nitrogen for three times and stirred for 45 min at room temperature. The resulting mixture was diluted with NaHCO₃ (aq.) (100 mL) and extracted with EA (3×150 mL). The residue was basified to pH 9 with NaOH (aq.). The combined organic layers was washed with brine (2×100 mL), dried over anhydrous Na₂SO₄. After filtration, the filtrate was concentrated under reduced pressure. The residue was purified by silica gel column chromatography, eluted with PE/EA (1/1). The fractions contained desired product were combined and concentrated to afford tert-butyl (3S)-3-{8-chloroimidazo[1,5-a]pyrazin-3-yl}pyrrolidine-1-carboxylate (12.70 g, 67%) as a light yellow oil. MS ESI calculated for C₁₅H₁₉ClN₄O₂ [M+H]⁺, 323.12, found 323.00.

### Step 3: Tert-butyl (3S)-3-{8-chloro-1-iodoimidazo[1,5-a]pyrazin-3-yl}pyrrolidine-1-carboxylate

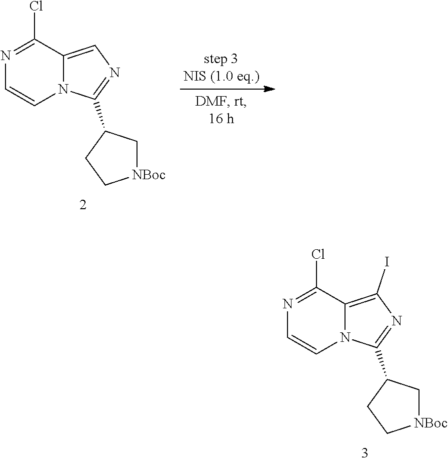 [SMILES: CC(C)(C)OC(=O)N1CC[C@H](c2ncc3c(Cl)nccn23)C1 ; CC(C)(C)OC(=O)N1CC[C@H](c2nc(I)c3c(Cl)nccn23)C1]

To a stirred solution of tert-butyl (3S)-3-{8-chloroimidazo[1,5-a]pyrazin-3-yl}pyrrolidine-1-carboxylate (12.00 g, 37.17 mmol) in DMF (60.00 mL) was added NIS (8.36 g, 37.17 mmol) dropwise at room temperature. The reaction mixture was degassed with nitrogen for three times and stirred for 16 h at room temperature. The resulting mixture was diluted with water (100 mL) and extracted with EA (3×200 mL). The combined organic layers was washed with brine (2×50 mL), dried over anhydrous Na₂SO₄. After filtration, the filtrate was concentrated under reduced pressure. The residue was purified by silica gel column chromatography, eluted with PE/EA (1/1). The fractions contained desired product were concentrated to afford tert-butyl (3S)-3-{8-chloro-1-iodoimidazo[1,5-a]pyrazin-3-yl}pyrrolidine-1-carboxylate (15.00 g, 89%) as a light yellow oil. MS ESI calculated for C₁₅H₁₈ClIN₄O₂ [M+H]⁺, 449.02, found 448.90; ¹H NMR (400 MHz, DMSO-d₆) δ 9.71 (d, J=45.5 Hz, 2H), 3.90-3.51 (m, 4H), 3.46-3.42 (m, 1H), 2.25 (d, J=35.0 Hz, 2H), 1.42 (s, 9H).

### Step 4: Tert-butyl (3S)-3-{8-amino-1-iodoimidazo[1,5-a]pyrazin-3-yl}pyrrolidine-1-carboxylate

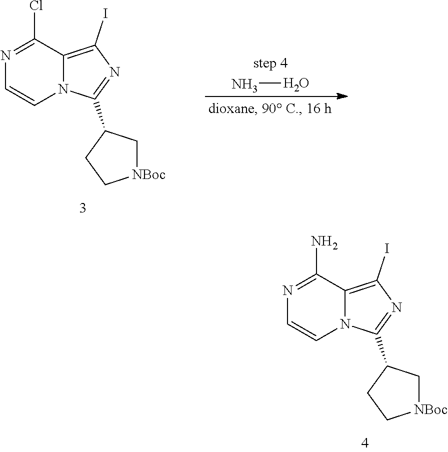

To a stirred solution of tert-butyl (3S)-3-{8-chloro-1-iodoimidazo[1,5-a]pyrazin-3-yl}pyrrolidine-1-carboxylate (8.00 g, 17.83 mmol) in 1,4-dioxane (16.00 mL) was added NH₃H₂O (64.00 mL) dropwise at room temperature. The reactions mixture was degassed with nitrogen for three times and stirred for 16 h at 90° C. The resulting mixture was dissolved in water (20 mL) and extracted with EA (3×100 mL). The combined organic layers was washed with brine (2×50 mL), dried over anhydrous Na₂SO₄. After filtration, the filtrate was concentrated under reduced pressure. The residue was purified by silica gel column chromatography, eluted with DCM/MeOH (20/1). The fractions contained desired product were combined and concentrated to afford tert-butyl (3S)-3-{8-amino-1-iodoimidazo[1,5-a]pyrazin-3-yl}pyrrolidine-1-carboxylate (3.00 g, 39%) as a yellow oil. MS ESI calculated for C₁₅H₂₀IN₅O₂ [M+H]⁺, 430.07, found 430.00; ¹H NMR (400 MHz, DMSO-d₆) δ 7.27 (s, 1H), 7.09 (d, J=5.1 Hz, 1H), 5.79 (s, 2H), 3.96-3.81 (m, 1H), 3.69-3.65 (m, 3H), 3.48 (q, J=9.0 Hz, 1H), 2.53-2.15 (m, 2H), 1.49 (s, 9H).

### Step 5: 1-iodo-3-[(3S)-Pyrrolidin-3-yl]imidazo[1,5-a]pyrazin-8-amine hydrochloride

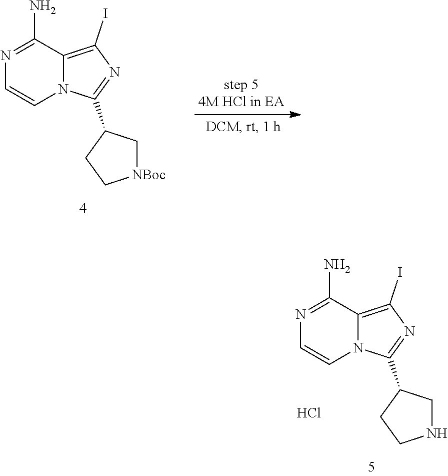 [SMILES: CC(C)(C)OC(=O)N1CC[C@H](c2nc(I)c3c(N)nccn23)C1 ; Nc1nccn2c([C@H]3CCNC3)nc(I)c12 ; Cl]

To a stirred solution of tert-butyl (3S)-3-{8-amino-1-iodoimidazo[1,5-a]pyrazin-3-yl}pyrrolidine-1-carboxylate (0.76 mg, 1.77 mmol) in DCM (7.00 mL) was added HCl in EA (4 M) (7.00 mL, 28.00 mmol) dropwise at 0° C. The reaction mixture was degassed with nitrogen for three times and stirred for 1 h at room temperature. The resulting mixture was concentrated under reduced pressure. The fractions contained desired product were combined and concenteated to afford 1-iodo-3-[(3S)-pyrrolidin-3-yl]imidazo[1,5-a]pyrazin-8-amine hydrochloride (0.70 g, crude) as an off-white solid. MS ESI calculated for C₁₀H₁₃ClIN₅ [M+H−HCl]⁺, 330.01, found 329.90.

### Step 6: 1-[(3S)-3-{8-Amino-1-iodoimidazo[1,5-a]pyrazin-3-yl}pyrrolidin-1-yl]prop-2-en-1-one

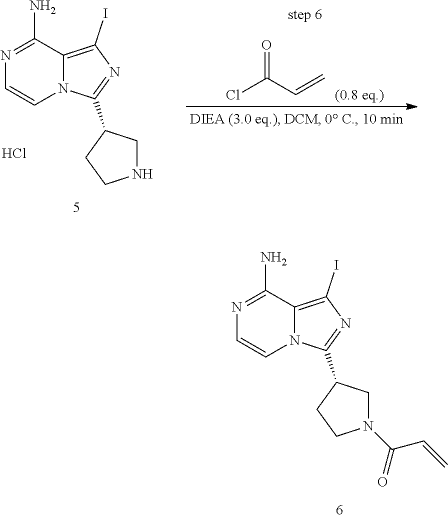

To a stirred solution of 1-iodo-3-[(3S)-pyrrolidin-3-yl]imidazo[1,5-a]pyrazin-8-amine hydrochloride (0.70 g, 2.12 mmol) in DCM (7.00 mL) was added DIEA (0.82 g, 6.38 mmol) and acryloyl chloride (6.81 mL, 1.70 mmol) dropwise at 0° C. The reaction mixture was degassed with nitrogen for three times and stirred for 10 min at 0° C. The reaction was quenched with MeOH (2 mL). The resulting mixture was dissolved in water (5 mL) and extracted with DCM (3×30 mL). The combined organic layers was washed with brine (2×30 mL), dried over anhydrous Na₂SO₄. After filtration, the filtrate was concentrated under reduced pressure. The residue was purified by silica gel column chromatography, eluted with DCM/MeOH (20/1). The fractions contained desired product were combined and concentrated to afford 1-[(3S)-3-{8-amino-1-iodoimidazo[1,5-a]pyrazin-3-yl}pyrrolidin-1-yl]prop-2-en-1-one (0.40 g, 49%) as a light yellow oil. MS ESI calculated for C₁₃H₁₄IN₅O [M+H]⁺, 384.02, found 384.20.

### Step 7: 1-[(3S)-3-{8-Amino-1-[2-(1-cyclopropyl-6-fluoro-1,3-benzodiazol-5-yl)ethynyl]imidazo[1,5-a]pyrazin-3-yl}pyrrolidin-1-yl]prop-2-en-1-one

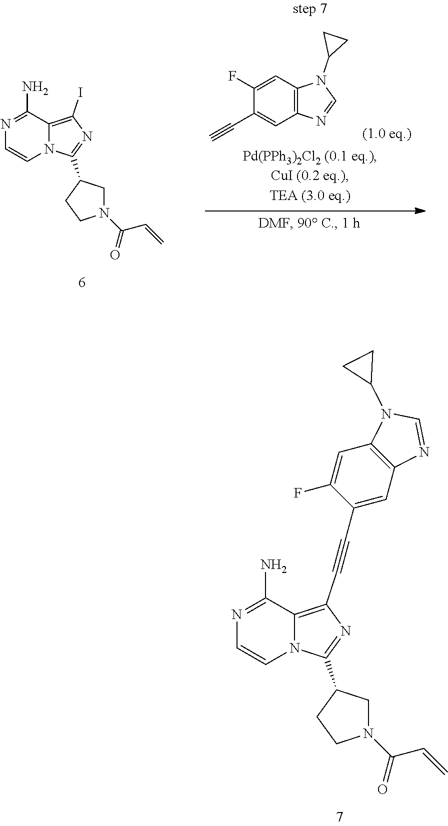 [SMILES: C=CC(=O)N1CC[C@H](c2nc(I)c3c(N)nccn23)C1 ; C#Cc1cc2ncn(C3CC3)c2cc1F ; "(1.0 eq.)" ; C=CC(=O)N1CC[C@H](c2nc(C#Cc3cc4ncn(C5CC5)c4cc3F)c3c(N)nccn23)C1]

To a stirred solution of 1-[(3S)-3-{8-amino-1-iodoimidazo[1,5-a]pyrazin-3-yl}pyrrolidin-1-yl]prop-2-en-1-one (0.35 g, 0.91 mmol), 1-cyclopropyl-5-ethynyl-6-fluoro-1,3-benzodiazole (0.19 g, 0.91 mmol), Pd(PPh₃)₂Cl₂ (64.11 mg, 0.09 mmol) and CuI (34.79 mg, 0.18 mmol) in DMF (7.00 mL) was added TEA (0.27 g, 2.73 mmol) dropwise at room temperature. The reaction mixture was degassed with nitrogen for three times and stirred for 1 h at 90° C. The resulting mixture was concentrated under reduced pressure. The residue was purified by silica gel column chromatography, eluted with DCM/MeOH (20/1) to afford the crude product which was further purified by reverse phase flash with the following conditions: Column: XBridge Shield RP18 OBD Column, 30×150 mm, 5 μm; Mobile phase A: water (10 mmol/L NH₄HCO₃), Mobile phase B: ACN; Flow rate: 60 mL/min; Gradient: 20% B to 35% B in 8 min; Wave length: 254 nm; RT1: 7.05 min. The fractions contained desired product were combined and concentrated to afford 1-[(3S)-3-{8-amino-1-[2-(1-cyclopropyl-6-fluoro-1,3-benzodiazol-5-yl)ethynyl]imidazo[1,5-a]pyrazin-3-yl}pyrrolidin-1-yl]prop-2-en-1-one (0.14 g, 35%) as a white solid. MS ESI calculated for C₂₅H₂₂FN₇O [M+H]⁺, 456.19, found 456.35; ¹H NMR (400 MHz, Chloroform-d) δ 8.10-7.90 (m, 2H), 7.36-7.31 (m, 1H), 7.27-6.83 (m, 2H), 6.79-6.33 (m, 2H), 6.03 (s, 2H), 5.78-5.67 (m, 1H), 4.39-3.46 (m, 5H), 3.38-3.32 (m, 1H), 2.84-2.21 (m, 2H), 1.33-1.11 (m, 2H), 1.14-0.75 (m, 2H).

### Example 202: 1-[(2R,4R)-4-{8-Amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one

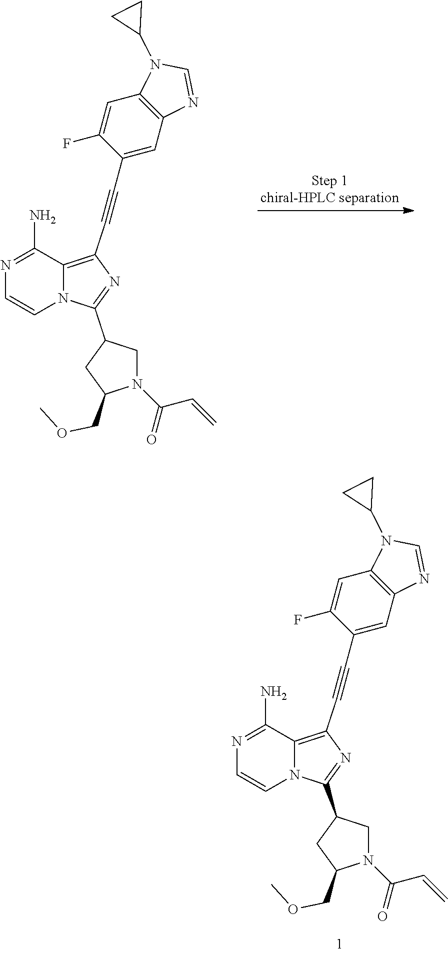 [SMILES: C=CC(=O)N1CC(c2nc(C#Cc3cc4ncn(C5CC5)c4cc3F)c3c(N)nccn23)C[C@@H]1COC ; C=CC(=O)N1C[C@H](c2nc(C#Cc3cc4ncn(C5CC5)c4cc3F)c3c(N)nccn23)C[C@@H]1COC]

The residue product from last step (1-[(2R)-4-{8-amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one) was purified by Chiral-HPLC chromatography with the following conditions: Column: CHIRAL ART Cellulose-SB, 2×25 cm, 5 μm; Mobile phase A: Hex (0.5% 2 M NH₃-MeOH)-HPLC, Mobile phase B:MeOH:EtOH=1:1-HPLC; Flow rate: 20 mL/min; Gradient: 50% B to 50% B in 16.5 min; Wave length: 220/254 nm; RT2: 14.52 min. The fractions contained desired product were combined and concentrated to afford 1-[(2R,4R)-4-{8-amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one (56.80 mg, 7%) as an off-white solid. MS ESI calculated for C₂₇H₂₆FN₇O₂ [M+H]⁺, 500.21, found 500.40; ¹H NMR (400 MHz, CDCl₃) δ 8.03-7.94 (m, 2H), 7.35 (d, J=9.1 Hz, 1H), 7.11 (d, J=5.2 Hz, 1H), 6.83-6.58 (m, 1H), 6.57-6.35 (m, 2H), 5.75 (d, J=10.1 Hz, 1H), 4.71-4.35 (m, 1H), 4.14-4.09 (m, 1H), 3.77 (d, J=9.0 Hz, 1H), 3.70-3.46 (m, 3H), 3.41-3.39 (m, 1H), 3.37 (d, J=5.3 Hz, 3H), 2.81-2.52 (m, 2H), 1.29-1.19 (m, 2H), 1.14-1.04 (m, 2H).

### Example 203: 1-[(2R,4S)-4-{8-Amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one

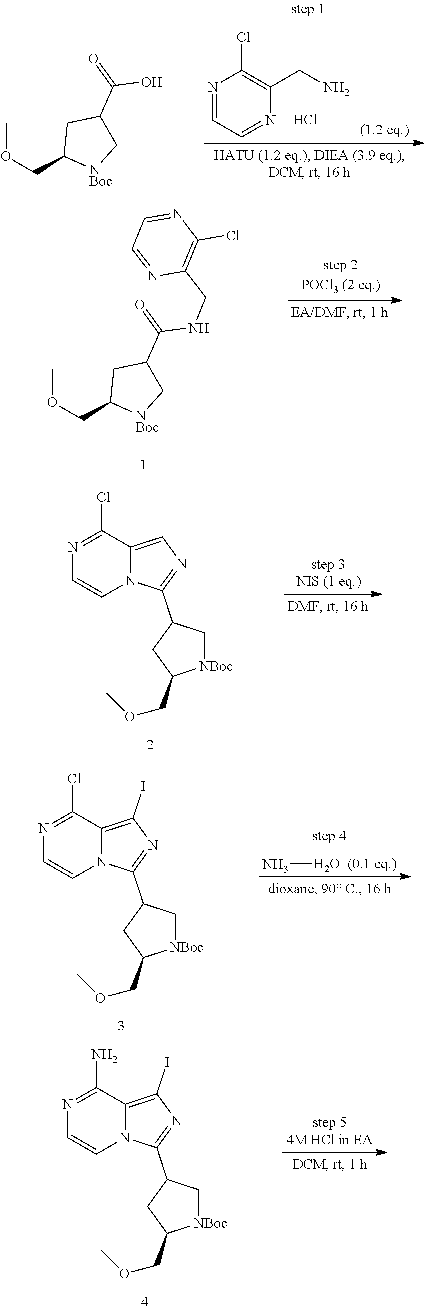

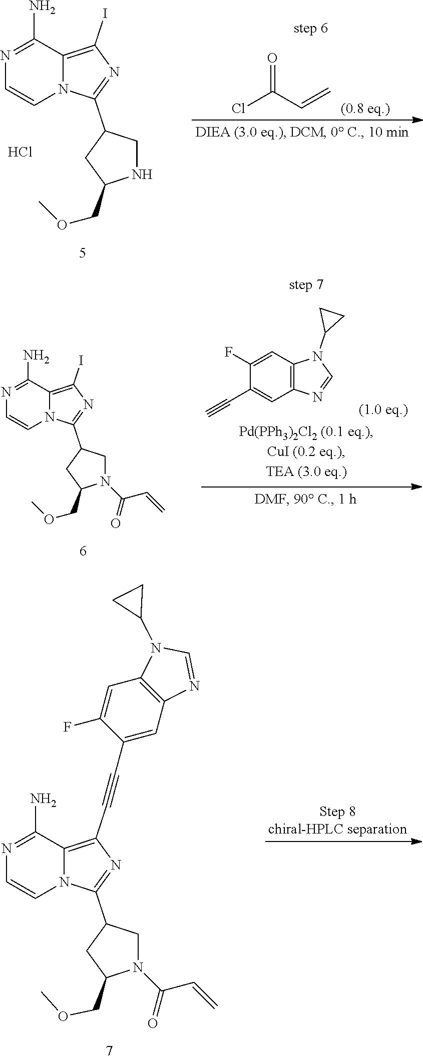

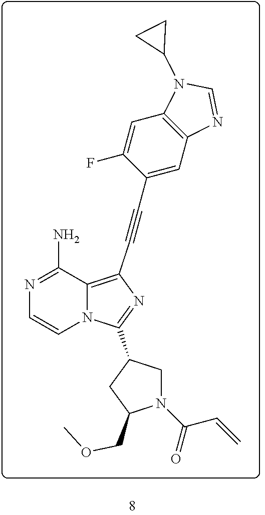 [SMILES: C=CC(=O)N1C[C@@H](c2nc(C#Cc3cc4ncn(C5CC5)c4cc3F)c3c(N)nccn23)C[C@@H]1COC]

### Step 1: Tert-butyl (2R)-4-{[(3-chloropyrazin-2-yl)methyl]carbamoyl}-2-(methoxymethyl)pyrrolidine-1-carboxylate

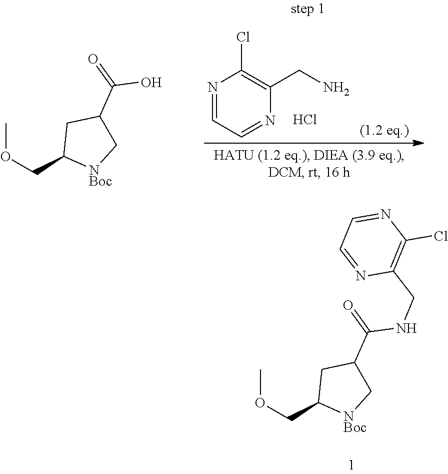 [SMILES: COC[C@H]1CC(C(=O)O)CN1C(=O)OC(C)(C)C ; NCc1nccnc1Cl ; Cl ; COC[C@H]1CC(C(=O)NCc2nccnc2Cl)CN1C(=O)OC(C)(C)C]

To a stirred mixture of (5R)-1-(tert-butoxycarbonyl)-5-(methoxymethyl)pyrrolidine-3-carboxylic acid (5.98 g, 23.05 mmol), HATU (10.67 g, 28.05 mmol) and 1-(3-chloropyrazin-2-yl)methanamine hydrochloride (5.00 g, 27.77 mmol) in DCM (0.43 L) was added DIEA (11.85 g, 91.65 mmol) dropwise at room temperature under nitrogen atmosphere. The reaction mixture was degassed with nitrogen for three times and stirred for 16 h at room temperature. The resulting mixture was quenched by water (100 mL) and extracted with DCM (3×100 mL). The combined organic layers was washed with brine (2×50 mL), dried over anhydrous Na₂SO₄. After filtration, the filtrate was concentrated under reduced pressure. The residue was purified by silica gel column chromatography, eluted with DCM/MeOH (10/1). The fractions contained desired product were combined and concentrated to afford tert-butyl (2R)-4-{[(3-chloropyrazin-2-yl)methyl]carbamoyl}-2-(methoxymethyl)pyrrolidine-1-carboxylate (9.10 g, 85%) as a yellow oil. MS ESI calculated for C₁₇H₂₅ClN₄O₄ [M+H]⁺, 385.16, found 385.30.

### Step 2: Tert-butyl (2R)-4-{8-chloroimidazo[1,5-a]pyrazin-3-yl}-2-(methoxymethyl)pyrrolidine

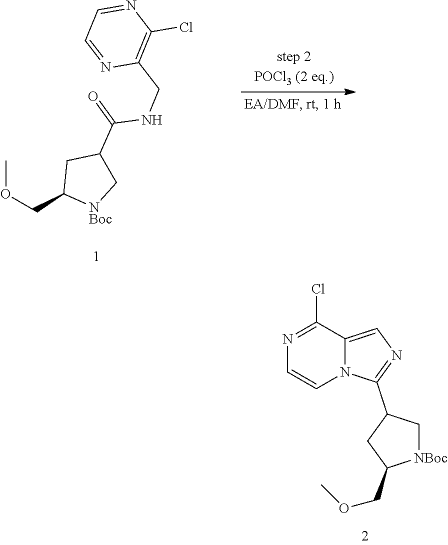 [SMILES: COC[C@H]1CC(C(=O)NCc2nccnc2Cl)CN1C(=O)OC(C)(C)C ; COC[C@H]1CC(c2ncc3c(Cl)nccn23)CN1C(=O)OC(C)(C)C]

To a stirred solution of tert-butyl (2R)-4-{[(3-chloropyrazin-2-yl)methyl]carbamoyl}-2-(methoxymethyl)pyrrolidine-1-carboxylate (9.00 g, 23.38 mmol) in DMF (9.00 mL) and EA (180.00 mL) was added POCl₃ (7.17 g, 46.77 mmol) dropwise at 0° C. under nitrogen atmosphere. The reaction mixture was degassed with nitrogen for three times and stirred for 1 h at room temperature. The reaction mixture was diluted with NaHCO₃ (aq.) (100 mL) and extracted with EA (4×150 mL). The residue was basified to pH 10 with NaOH (aq.). The combined organic layers was washed with brine (2×50 mL), dried over anhydrous Na₂SO₄. After filtration, the filtrate was concentrated under reduced pressure. The residue was purified by silica gel column chromatography, eluted with PE/EA (1/1). The fractions contained desired product were combined and concentrated to afford tert-butyl (2R)-4-{8-chloroimidazo[1,5-a]pyrazin-3-yl}-2-(methoxymethyl)pyrrolidine (4.00 g, 64%) as a light yellow oil. MS ESI calculated for C₁₇H₂₃ClN₄O₃ [M+H]⁺, 367.15, found, 367.10; ¹H NMR (400 MHz, Chloroform-d) δ 7.83-7.58 (m, 2H), 7.36-7.34 (m, 1H), 4.32-3.38 (m, 9H), 2.65-2.26 (m, 2H), 1.46 (s, 9H).

### Step 3: Tert-butyl (2R)-4-{8-chloro-1-iodoimidazo[1,5-a]pyrazin-3-yl}-2-(methoxymethyl)pyrrolidine-1-carboxylate

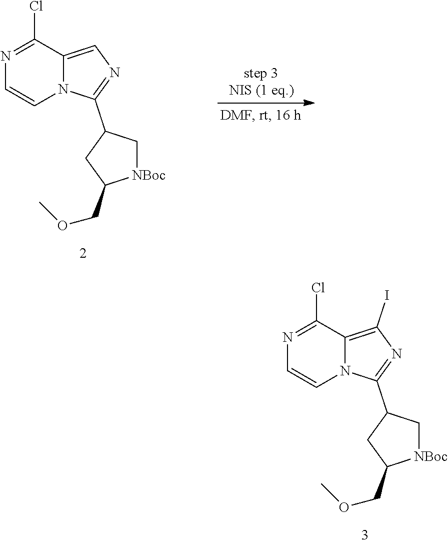 [SMILES: COC[C@H]1CC(c2ncc3c(Cl)nccn23)CN1C(=O)OC(C)(C)C ; COC[C@H]1CC(c2nc(I)c3c(Cl)nccn23)CN1C(=O)OC(C)(C)C]

To a stirred solution of tert-butyl (2R)-4-{8-chloroimidazo[1,5-a]pyrazin-3-yl}-2-(methoxymethyl)pyrrolidine (4.00 g, 10.90 mmol) in DMF (20.00 mL) was added NIS (2.45 g, 10.90 mmol) dropwise at room temperature. The reaction mixture was degassed with nitrogen for three times and stirred for 16 h at room temperature. The resulting mixture was diluted with water (100 mL) and extracted with EA (5×100 mL). The combined organic layers was washed with brine (2×50 mL), dried over anhydrous Na₂SO₄. After filtration, the filtrate was concentrated under reduced pressure. The residue was purified by silica gel column chromatography, eluted with PE/EA (1/1). The fractions contained desired product were combined and concentrated to afford tert-butyl (2R)-4-{8-chloro-1-iodoimidazo[1,5-a]pyrazin-3-yl}-2-(methoxymethyl)pyrrolidine-1-carboxylate (4.50 g, 83%) as a light yellow oil. MS ESI calculated for C₁₇H₂₂ClIN₄O₃ [M+H]⁺, 493.04, found 493.15; ¹H NMR (400 MHz, CDCl₃) δ 7.37-7.39 (m, 1H), 4.14-4.19 (m, 1H), 3.85-3.94 (m, 1H), 3.70 (d, J=28.0 Hz, 2H), 3.50-3.43 (m, 1H), 3.40 (d, J=12.2 Hz, 3H), 2.97 (s, 1H), 2.69-2.26 (m, 2H), 1.49 (s, 9H).

### Step 4: Tert-butyl (2R)-4-{8-amino-1-iodoimidazo[1,5-a]pyrazin-3-yl}-2-(methoxymethyl)pyrrolidine-1-carboxylate

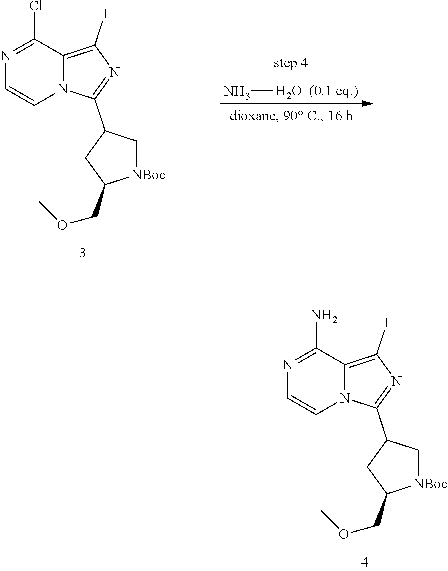

To a stirred solution of tert-butyl (2R)-4-{8-chloro-1-iodoimidazo[1,5-a]pyrazin-3-yl}-2-(methoxymethyl)pyrrolidine-1-carboxylate (4.60 g, 9.33 mmol) in 1,4-dioxane (9.20 mL) was added NH₃H₂O (36.80 mL, 0.94 mol) dropwise at room temperature. The reaction mixture was degassed with nitrogen for three times and stirred for 16 h at 90° C. The resulting mixture was diluted with water (20 mL) and extracted with EA (2×100 mL). The combined organic layers was washed with brine (2×50 mL), dried over anhydrous Na₂SO₄. After filtration, the filtrate was concentrated under reduced pressure. The residue was purified by silica gel column chromatography, eluted with DCM/MeOH (20/1). The fractions contained desired product were combined and concentrated to afford tert-butyl (2R)-4-{8-amino-1-iodoimidazo[1,5-a]pyrazin-3-yl}-2-(methoxymethyl)pyrrolidine-1-carboxylate (2.70 g, 61%) as a yellow solid. MS ESI calculated for C₁₇H₂₄IN₅O₃ [M+H]⁺, 474.09, found 474.20; ¹H NMR (400 MHz, CDCl₃) δ 7.28 (d, J=10.6 Hz, 1H), 7.07 (t, J=4.1 Hz, 1H), 5.84 (s, 2H), 4.18 (s, 1H), 3.83-3.79 (m, 1H), 3.77-3.61 (m, 2H), 3.51-3.41 (m, 2H), 3.39 (d, J=14.3 Hz, 3H), 2.65-2.19 (m, 2H), 1.48 (s, 9H).

### Step 5: 1-Iodo-3-[(5R)-5-(methoxymethyl)pyrrolidin-3-yl]imidazo[1,5-a]pyrazin-8-amine hydrochloride

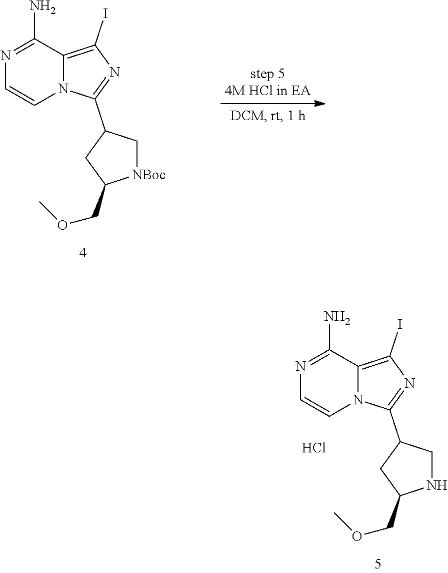 [SMILES: COC[C@H]1CC(c2nc(I)c3c(N)nccn23)CN1C(=O)OC(C)(C)C ; COC[C@H]1CC(c2nc(I)c3c(N)nccn23)CN1 ; Cl]

To a stirred solution of tert-butyl (2R)-4-{8-amino-1-iodoimidazo[1,5-a]pyrazin-3-yl}-2-(methoxymethyl)pyrrolidine-1-carboxylate (1.00 g, 2.11 mmol) in DCM (10.00 mL) was added HCl (4 M) in EA (10.00 mL, 40.00 mmol) dropwise at 0° C. The reaction mixture was degassed with nitrogen for three times and stirred for 1 h at room temperature. The resulting mixture was concentrated under reduced pressure. The desired product 1-iodo-3-[(5R)-5-(methoxymethyl)pyrrolidin-3-yl]imidazo[1,5-a]pyrazin-8-amine hydrochloride (0.90 g, crude) was used in the next step directly without further purification. MS ESI calculated for C₁₂H₁₇ClIN₅O [M+H−HCl]⁺, 374.04, found 374.20.

### Step 6: 1-[(2R)-4-{8-Amino-1-iodoimidazo[1,5-a]pyrazin-3-yl}-2-(methoxymethyl)pyrrolidin-1-yl]prop-2-en-1-one

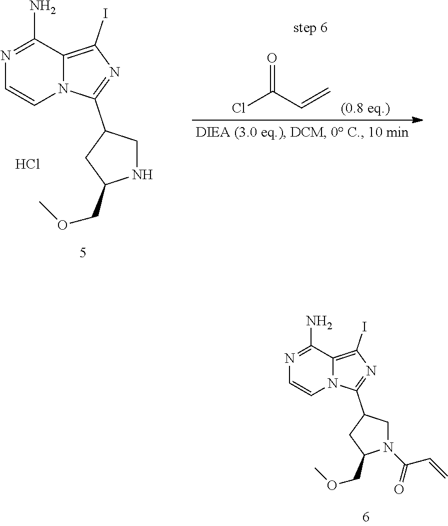

To a stirred solution of 1-iodo-3-[(5R)-5-(methoxymethyl)pyrrolidin-3-yl]imidazo[1,5-a]pyrazin-8-amine hydrochloride (0.92 g, 2.45 mmol) in DCM (9.18 mL) were added DIEA (0.95 g, 7.37 mmol) and acryloyl chloride (7.87 mL, 1.96 mmol) dropwise at 0° C. The reaction mixture was stirred for 10 min at 0° C. under nitrogen atmosphere. The resulting mixture was quenched by the addition of MeOH (2 mL) and extracted with DCM (3×30 mL). The combined organic layers was washed with brine (2×30 mL), dried over anhydrous Na₂SO₄ and filtered. The filtrate was concentrated under reduced pressure. The residue was purified by silica gel column chromatography, eluted with DCM/MeOH (20/1). The fractions contained desired product were combined and concentrated to afford 1-[(2R)-4-{8-amino-1-iodoimidazo[1,5-a]pyrazin-3-yl}-2-(methoxymethyl)pyrrolidin-1-yl]prop-2-en-1-one (0.60 g, 57%) as a light yellow oil. MS ESI calculated for C₁₅H₁₈IN₅O₂ [M+H]⁺, 428.05, found 428.05.

### Step 7: 1-[(2R)-4-{8-Amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one

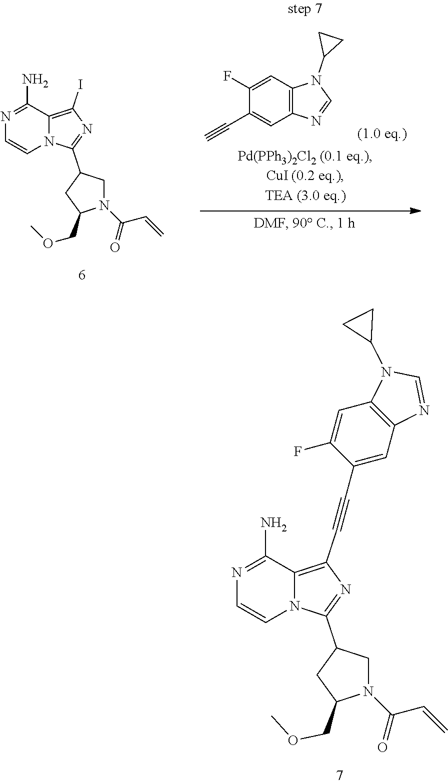 [SMILES: C=CC(=O)N1CC(c2nc(I)c3c(N)nccn23)C[C@@H]1COC ; C#Cc1cc2ncn(C3CC3)c2cc1F ; "(1.0 eq.)" ; C=CC(=O)N1CC(c2nc(C#Cc3cc4ncn(C5CC5)c4cc3F)c3c(N)nccn23)C[C@@H]1COC]

To a stirred mixture of 1-[(2R)-4-{8-amino-1-iodoimidazo[1,5-a]pyrazin-3-yl}-2-(methoxymethyl)pyrrolidin-1-yl]prop-2-en-1-one (0.60 g, 1.40 mmol), 1-cyclopropyl-5-ethynyl-6-fluoro-1,3-benzodiazole (0.29 g, 1.47 mmol), Pd(PPh₃)₂Cl₂ (98.57 mg, 0.14 mmol) and CuI (53.49 mg, 0.28 mmol) in DMF (12.00 mL) was added TEA (0.43 g, 4.21 mmol) dropwise at room temperature. The reaction mixture was degassed with nitrogen for three times and stirred for 1 h at 90° C. The resulting mixture was concentrated under reduced pressure. The residue was purified by silica gel column chromatography, eluted with DCM/MeOH (20/1). The fractions contained desired product were combined and concentrated to afford 1-[(2R)-4-{8-amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one (0.35 g, 49%). MS ESI calculated for C₂₇H₂₆FN₇O₂ [M+H]⁺, 500.21, found 500.35.

### Step 8: 1-[(2R,4S)-4-{8-Amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one

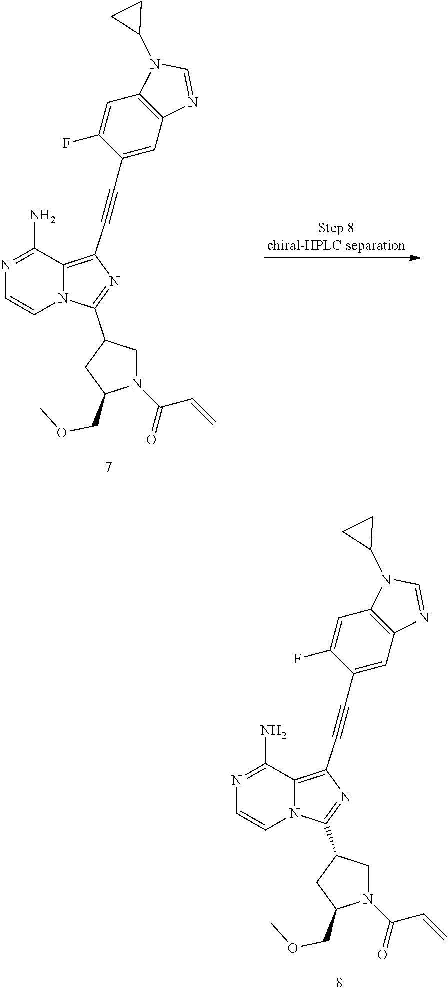 [SMILES: C=CC(=O)N1CC(c2nc(C#Cc3cc4ncn(C5CC5)c4cc3F)c3c(N)nccn23)C[C@@H]1COC ; C=CC(=O)N1C[C@@H](c2nc(C#Cc3cc4ncn(C5CC5)c4cc3F)c3c(N)nccn23)C[C@@H]1COC]

The residue product from last step (1-[(2R)-4-{8-amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one) was purified by Chiral-HPLC chromatography with the following conditions: Column: CHIRAL ART Cellulose-SB, 2×25 cm, 5 μm; Mobile phase A: Hex (0.5% 2 M NH₃-MeOH)-HPLC, Mobile phase B: MeOH:EtOH=1:1-HPLC; Flow rate: 20 mL/min; Gradient: 50% B to 50% B in 16.5 min; Wave length: 220/254 nm; RT1: 11.20 min. The fractions contained desired product were combined and concentrated to afford 1-[(2R,4S)-4-{8-amino-1-[2-(1-cyclopropyl-6-fluoro-1,3-benzodiazol-5-yl)ethynyl]imidazo[1,5-a]pyrazin-3-yl}-2-(methoxymethyl)pyrrolidin-1-yl]prop-2-en-1-one (0.14 g, 21%) as an off-white solid. MS ESI calculated for C₂₇H₂₆FN₇O₂ [M+H]⁺, 500.21, found 500.40; ¹H NMR (400 MHz, CDCl₃) δ 7.97 (t, J=3.1 Hz, 2H), 7.30 (s, 1H), 7.27-7.12 (m, 2H), 6.60-6.31 (m, 2H), 6.18-5.88 (m, 2H), 5.72-5.78 (m, 1H), 4.65-4.36 (m, 1H), 4.18-4.04 (m, 2H), 3.95-3.79 (m, 1H), 3.62-3.49 (m, 1H), 3.47-3.32 (m, 4H), 2.54-2.25 (m, 2H), 1.25-0.97 (m, 4H).

### II. Biological Evaluation

### Example 1: FGFR2 Kinase Protocol—Condition 1

The Reaction Biology HotSpot assay platform (http://www.reactionbiology.com) was used to measure kinase/inhibitor interactions as described previously (Anastassiadis et al., 2011). In brief, for each reaction, kinase and substrate were mixed in a buffer containing 20 mM HEPES (pH 7.5), 10 mM MgCl2, 1 mM EGTA, 0.02% Brij35, 0.02 mg/mL BSA, 0.1 mM Na3VO4, 2 mM DTT, and 1% DMSO. All compounds were in powder form and freshly solubilized in DMSO for assays. Compounds were then added to each reaction mixture via acoustic dispense using an ECHO 550 nanoliter dispenser. For Human FGFR2 wildtype testing, a peptide substrate, poly[Glu:Tyr] (4:1) was used to promote the reaction at a concentration of 0.2 mg/ml. For Human FGFR2 (V564F) testing, a peptide substrate, poly[Glu:Tyr] (4:1) was used to promote the reaction at a concentration of 0.2 mg/ml. ATP concentration for all assays was maintained at 100 micromolar. Compounds were tested in 10-dose IC50 mode with a 3-fold serial dilution. After a 20-min incubation, ATP (Sigma-Aldrich, St. Louis, MO 63178) and [g33P] ATP (specific activity 10 microCi/microliter) purchased at PerkinElmer (Boston, MA, 02118 Cat #BLU 003H250UC) were added at a final total concentration of 10 mM. Reactions were carried out at room temperature for 2 hr and spotted onto P81 ion exchange cellulose chromatography paper (Reaction Biology). Filter paper was washed in 0.75% phosphoric acid to remove unincorporated ATP. The percent remaining kinase activity relative to a vehicle-containing (DMSO) kinase reaction was calculated for each kinase/inhibitor pair. IC50 values were calculated using Dotmatics Knowledge Solutions Studies curve fitting (Dotmatics, Bishops Stortford, UK, CM23).

### Example 2: FGFR2 Kinase Protocol—Condition 2

The Reaction Biology HotSpot assay platform (http://www.reactionbiology.com) was used to measure kinase/inhibitor interactions as described previously (Anastassiadis et al., 2011). In brief, for each reaction, kinase and substrate were mixed in a buffer containing 20 mM HEPES (pH 7.5), 10 mM MgCl2, 1 mM EGTA, 0.02% Brij35, 0.02 mg/mL BSA, 0.1 mM Na3VO4, 2 mM DTT, and 1% DMSO. All compounds were in powder form and freshly solubilized in DMSO for assays. Compounds were then added to each reaction mixture via acoustic dispense using an ECHO 550 nanoliter dispenser. For Human FGFR2 wildtype testing, a peptide substrate, poly[Glu:Tyr] (4:1) was used to promote the reaction at a concentration of 0.2 mg/ml. For Human FGFR2 (V564F) testing, a peptide substrate, poly[Glu:Tyr] (4:1) was used to promote the reaction at a concentration of 0.2 mg/ml. ATP concentration for all assays was maintained at 100 micromolar. Compounds were tested in 10-dose IC50 mode with a 3-fold serial dilution. Compounds were added to plates and ATP was added directly to the wells. ATP (Sigma-Aldrich, St. Louis, MO 63178) and [g33P] ATP (specific activity 10 microCi/microliter) purchased at PerkinElmer (Boston, MA, 02118 Cat #BLU 003H250UC) were added at a final total concentration of 100 microM to each well with no preincubation. Reactions were carried out at room temperature for 2 hr and spotted onto P81 ion exchange cellulose chromatography paper (Reaction Biology). Filter paper was washed in 0.75% phosphoric acid to remove unincorporated ATP. The percent remaining kinase activity relative to a vehicle-containing (DMSO) kinase reaction was calculated for each kinase/inhibitor pair. IC50 values were calculated using Dotmatics Knowledge Solutions Studies curve fitting (Dotmatics, Bishops Stortford, UK, CM23).

TABLE 3

| Synthetic Chemistry | FGFR2 IC₅₀ | FGFR2 IC₅₀ |
|---|---|---|
| Example | Condition 1 | Condition 2 |
| 1 | A | — |
| 2 | A | — |
| 3 | A | A |
| 4 | A | — |
| 5 | A | — |
| 6 | A | — |
| 7 | A | — |
| 8 | A | — |
| 9 | B | — |
| 10 | A | — |
| 11 | A | — |
| 12 | A | A |
| 13 | A | — |
| 14 | A | — |
| 15 | A | — |
| 16 | A | A |
| 17 | A | — |
| 18 | A | — |
| 19 | A | — |
| 20 | A | — |
| 21 | A | — |
| 22 | A | — |
| 23 | A | — |
| 24 | A | — |
| 25 | A | — |
| 26 | A | — |
| 27 | A | — |
| 28 | C | C |
| 29 | A | B |
| 30 | A | B |
| 31 | A | B |
| 32 | — | A |
| 33 | — | A |
| 34 | — | A |
| 35 | — | B |
| 36 | — | B |
| 37 | — | A |
| 38 | — | C |
| 39 | — | A |
| 40 | — | A |
| 41 | — | B |
| 42 | — | B |
| 43 | — | A |
| 44 | — | A |
| 45 | — | B |
| 46 | — | A |
| 47 | — | A |
| 48 | — | A |
| 49 | — | ≥B |
| 50 | — | A |
| 51 | — | ≥B |
| 52 | — | A |
| 53 | — | A |
| 54 | — | A |
| 55 | — | A |
| 56 | — | A |
| 57 | — | A |
| 58 | — | A |
| 59 | — | A |
| 60 | — | A |
| 61 | — | A |
| 62 | — | A |
| 63 | — | A |
| 64 | — | A |
| 65 | — | A |
| 66 | — | A |
| 67 | — | A |
| 68 | — | A |
| 69 | — | A |
| 70 | — | A |
| 71 | — | A |
| 72 | — | A |
| 73 | — | A |
| 74 | — | A |
| 75 | — | A |
| 76 | — | A |
| 77 | — | A |
| 78 | — | A |
| 79 | — | A |
| 80 | — | A |
| 81 | — | A |
| 82 | — | A |
| 83 | — | A |
| 84 | — | A |
| 85 | — | A |
| 86 | — | A |
| 87 | — | A |
| 88 | — | A |
| 89 | — | A |
| 90 | — | A |
| 91 | — | A |
| 92 | — | A |
| 93 | — | A |
| 94 | — | A |
| 95 | — | A |
| 96 | — | A |
| 97 | — | A |
| 98 | — | A |
| 99 | — | A |
| 100 | — | A |
| 101 | — | A |
| 102 | — | A |
| 103 | — | A |
| 104 | — | A |
| 105 | — | A |
| 106 | — | A |
| 107 | — | A |
| 108 | — | A |
| 109 | — | A |
| 110 | — | A |
| 112 | — | A |
| 113 | — | A |
| 114 | — | B |
| 115 | — | A |
| 116 | — | A |
| 117 | — | A |
| 118 | — | A |
| 119 | — | A |
| 120 | — | A |
| 121 | — | A |
| 122 | — | A |
| 123 | — | A |
| 124 | — | A |
| 125 | — | A |
| 126 | — | A |
| 127 | — | A |
| 128 | — | A |
| 129 | — | A |
| 130 | — | A |
| 131 | — | A |
| 132 | — | A |
| 133 | — | A |
| 134 | — | A |
| 135 | — | A |
| 136 | — | A |
| 137 | — | A |
| 138 | — | A |
| 139 | — | A |
| 140 | — | A |
| 141 | — | A |
| 142 | — | A |
| 143 | — | A |
| 144 | — | A |
| 145 | — | A |
| 146 | — | A |
| 147 | — | A |
| 148 | — | A |
| 149 | — | A |
| 150 | — | A |
| 151 | — | A |
| 152 | — | A |
| 153 | — | A |
| 154 | — | A |
| 155 | — | A |
| 156 | — | A |
| 157 | — | A |
| 158 | — | A |
| 159 | — | A |
| 160 | — | A |
| 161 | — | A |
| 162 | — | A |
| 163 | — | A |
| 164 | — | A |
| 165 | — | A |
| 166 | — | A |
| 167 | — | A |
| 168 | — | A |
| 169 | — | A |
| 170 | — | A |
| 171 | — | A |
| 172 | — | A |
| 173 | — | A |
| 174 | — | A |
| 175 | — | A |
| 176 | — | A |
| 177 | — | A |
| 178 | — | A |
| 179 | — | A |
| 180 | — | A |
| 181 | — | A |
| 182 | — | A |
| 183 | — | A |
| 184 | — | A |
| 185 | — | A |
| 186 | — | A |
| 187 | — | A |
| 188 | — | A |
| 189 | — | A |
| 190 | — | A |
| 191 | — | A |
| 192 | — | A |
| 193 | — | A |
| 194 | — | A |
| 195 | — | A |
| 196 | — | A |
| 197 | — | A |
| 198 | — | >500 nM |
| 199 | — | A |
| 200 | — | >500 nM |
| 201 | — | B |
| 202 | — | >500 nM |
| 203 | — | A |

Note:
Biochemical assay IC₅₀ data are designated within the following ranges:
A: ≤0.10 μM
B: >0.10 μM to ≤1.0 μM
C: >1.0 μM to ≤10 μM
D: >10 μM to 30 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1. A compound, or pharmaceutically acceptable salt or solvate thereof, having the structure of Formula (I): 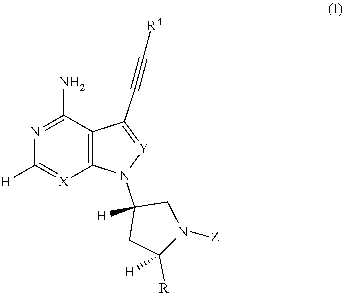
wherein,
X is C—H or N;
Y is C—H or N;
Z is selected from a group having the structure: 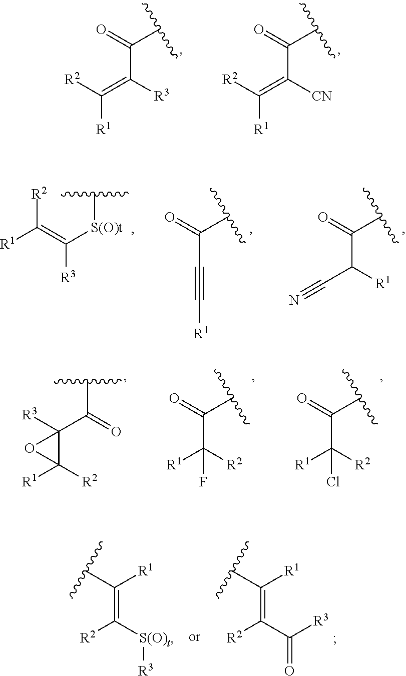
t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

2. The compound of claim 1, or a pharmaceutically acceptable salt or solvate thereof, wherein X is C—H, and Y is C—H.

3. The compound of claim 1, or a pharmaceutically acceptable salt or solvate thereof, wherein X is C—H, and Y is N.

4. The compound of claim 1, or a pharmaceutically acceptable salt or solvate thereof, wherein X is N, and Y is C—H.

5. The compound of claim 1, or a pharmaceutically acceptable salt or solvate thereof, wherein X is N, and Y is N.

6. The compound of claim 1, or a pharmaceutically acceptable salt or solvate thereof, wherein Z is

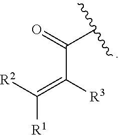

7. (canceled)

8. (canceled)

9. The compound of claim 1, or pharmaceutically acceptable salt or solvate thereof, wherein R² and R³ are hydrogen.

10.-15. (canceled)

16. The compound of claim 1, or a pharmaceutically acceptable salt or solvate thereof, wherein R⁴ is selected from an optionally substituted nitrogen-containing 9 or 10-atom heteroaryl is selected from optionally substituted benzimidazole, optionally substituted 1H-indazole, optionally substituted 2H-indazole, optionally substituted benzotriazole, optionally substituted benzoxazole, optionally substituted imidazo[4,5-c]pyridine, or optionally substituted imidazo[4,5-b]pyridine.

17. The compound of claim 1, or a pharmaceutically acceptable salt or solvate thereof, wherein R⁴ is selected from an optionally substituted nitrogen-containing 9 or 10-atom heteroaryl is selected from quinoline, quinoxaline, pyrazolo[1,5-a]pyrimidine, imidazo[1,2-a]pyridine, pyrazolo[1,5-a]pyridine, imidazo[1,2-a]pyrimidine, imidazo[1,2-b]pyridazine, or pyrazolo[1,5-a]pyridine.

18. The compound of claim 16, or a pharmaceutically acceptable salt or solvate thereof, wherein R⁴ is an optionally substituted benzimidazole.

19. The compound of claim 16, or a pharmaceutically acceptable salt or solvate thereof, wherein R⁴ is an optionally substituted 1H-indazole.

20. The compound of claim 16, or a pharmaceutically acceptable salt or solvate thereof, wherein R⁴ is an optionally substituted 2H-indazole.

21. The compound of claim 16, or a pharmaceutically acceptable salt or solvate thereof, wherein R⁴ is an optionally substituted benzoxazole, optionally substituted imidazo[4,5-c]pyridine, or optionally substituted imidazo[4,5-b]pyridine.

22. The compound of claim 16, or a pharmaceutically acceptable salt or solvate thereof, wherein optionally substituted nitrogen-containing 9 or 10-atom heteroaryl is optionally substituted with alkyl, cycloalkyl, or halogen.

23. The compound of claim 18, or a pharmaceutically acceptable salt or solvate thereof, wherein the optionally substituted benzimidazole is optionally substituted with alkyl, cycloalkyl, or halogen.

24. The compound of claim 19, or a pharmaceutically acceptable salt or solvate thereof, wherein the optionally substituted 1H-indazole is optionally substituted with alkyl, cycloalkyl, or halogen.

25. The compound of claim 20, or a pharmaceutically acceptable salt or solvate thereof, wherein the optionally substituted 2H-indazole is optionally substituted with alkyl, cycloalkyl, or halogen.

26. The compound of claim 1, or a pharmaceutically acceptable salt or solvate thereof, wherein R is hydrogen.

27. The compound of claim 1, or a pharmaceutically acceptable salt or solvate thereof, wherein R is optionally substituted C1-C6 alkyl.

28. The compound of claim 1, or a pharmaceutically acceptable salt or solvate thereof, wherein R is optionally substituted C3-C7 carbocyclyl.

29. The compound of claim 1, or a pharmaceutically acceptable salt or solvate thereof, wherein R is optionally substituted C3-C7 carbocyclylalkyl.

30. The compound of claim 1, or a pharmaceutically acceptable salt or solvate thereof, wherein R is optionally substituted C3-C7 heterocyclyl.

31. The compound of claim 1, or a pharmaceutically acceptable salt or solvate thereof, wherein R is optionally substituted C3-C7 heterocyclylalkyl.

32. The compound of claim 1, or a pharmaceutically acceptable salt or solvate thereof, wherein R is —CO₂R⁵.

33. The compound of claim 1, or a pharmaceutically acceptable salt or solvate thereof, wherein R is —CONHR⁵ or —CON(R⁵)₂.

34. The compound of claim 27, or a pharmaceutically acceptable salt or solvate thereof, wherein the optionally substituted C1-C6 alkyl is a C1-C3 alkyl substituted with a C1-C3 alkoxy.

35. A pharmaceutical composition comprising a compound of Formula (I), or pharmaceutically acceptable salt or solvate thereof, as described in claim 1.

36.-39. (canceled)

40. A method of treating cancer in a patient in need thereof comprising administering to the patient a compound of Formula (I) as described in claim 1, or pharmaceutically acceptable salt or solvate thereof.

41. A method of treating cancer in a patient in need thereof comprising administering to the patient a pharmaceutical composition comprising a compound of Formula (I) as described claim 1, or pharmaceutically acceptable salt or solvate thereof, and a pharmaceutically acceptable excipient.
